(19) United States
(12) Patent Application Publication
(10) Pub. No.: US 2025/0122174 A1
(43) Pub. Date: Apr. 17, 2025

(54) Heteroaryl Compounds As HPK1 Inhibitors And Methods Of Using Same

(71) Applicants: Shenzhen Yuanli Shenwan Innovative Medicine Co., Ltd., Pingshan District, Shenzhen (CN); FOSHAN IONOVA BIOTHERAPEUTICS CO., INC., Foshan, Guangdong (CN)

(72) Inventors: Yongkui Sun, Bridgewater, NJ (US); Weiguo Quan, Pingshan District, Shenzhen (CN); Weiping Ma, Pingshan District, Shenzhen (CN); Jianming Bao, Pingshan District, Shenzhen (CN); Koc Kan Ho, Pingshan District, Shenzhen (CN); Mengchao Shi, Pingshan District, Shenzhen (CN)

(21) Appl. No.: 18/728,660

(22) PCT Filed: Jan. 12, 2023
(86) PCT No.: PCT/US2023/060597

§ 371 Date: Jul. 12, 2024

Related U.S. Application Data
(60) Provisional application No. 63/299,000, filed on Jan. 12, 2022.
(60) Provisional application No. 63/418,888, filed on Oct. 24, 2022.

(30) Foreign Application Priority Data
Aug. 17, 2022 (WO) PCT/CN2022/112996

(51) Int. Cl. C07D 403/14; A61K 31/407; A61K 31/427; A61K 31/4439; A61K 31/444; A61K 31/4545; A61K 31/501; A61K 31/5383; A61K 45/06; C07D 401/14; C07D 405/14; C07D 471/10; C07D 487/04; C07D 487/08; C07D 487/10; C07D 491/107; C07D 498/04

(52) U.S. Cl. CPC C07D 403/14; A61K 31/407; A61K 31/427; A61K 31/4439; A61K 31/444; A61K 31/4545; A61K 31/501; A61K 31/5383; A61K 45/06; C07D 401/14; C07D 405/14; C07D 471/10; C07D 487/04; C07D 487/08; C07D 487/10; C07D 491/107; C07D 498/04

(57) ABSTRACT

Among others, the present invention provides heteroaryl compounds of Formula (I) or a pharmaceutically acceptable salt, ester, or prodrug thereof as HPK1 inhibitors, and methods of use thereof.

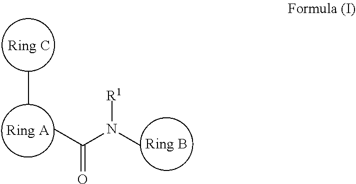

### CROSS-REFERENCE TO RELATED APPLICATIONS

This application claims the benefit of priority to U.S. application No. 63/299,000, filed on Jan. 12, 2022, international application number PCT/CN2022/112996, filed on Aug. 17, 2022, and U.S. application No. 63/418,888, filed on Oct. 24, 2022, the contents of all of which are incorporated herein by reference in their entirety.

### BACKGROUND OF THE INVENTION

Progenitor Kinase 1 (HPK1) is predominately expressed in hematopoietic cells and is functioning as a negative regulator in T cells and dendritic cells (DC). Abrogation of HPK1's kinase activity by genetically knocking in the kinase death (KD) enzyme demonstrated its involvement in anti-tumor immune response (Hernandes et. al., US 2016/0158360). Mechanistically, HPK1 KD decreases the phosphorylation of a downstream protein SLP76 in T cell, and in turn increase the pro-immunity cytokines IL-2 and IFNγ release upon anti-CD3 and anti-CD28 stimulation. Increase of tumor infiltrated lymphocytes (TIL) in the tumor microenvironment (TME) along with reduction of tumor growth was observed in a sarcoma model for mouse expressing the KD enzyme (Liu et. al., PLoS ONE 14(3): e0212670). Furthermore, small molecule HPK1 inhibitors have demonstrated anti-tumor activity as a single agent or in combination with anti-PD1 (Chen et. al., AACR Annual Meeting, June 2020, Poster 4513) or with anti-CTLA-4 (Ciccone et. al., AACR Annual Meeting, June 2020, Poster 942) antibodies in various cancer models.

It has been shown that HPK1 KD T cells are resistant to immune suppression exerted by PGE2 and adenosine, supporting the investigation of a synergistic anti-cancer effect with compounds inhibiting the PGE2 and adenosine pathways. For example, a HPK1 inhibitor in combination with an EP4 or an A2a/b inhibitor may show superior therapeutic benefit for treatment of cancer (Liu et. al., PLoS ONE 14(3): e0212670).

Given HPK1's role in modulating the activity in immune cells, small molecule inhibitors could be useful as a single agent or in combination with existing immunotherapies, targeted therapies, or compounds inhibiting the PGE2 or adenosine pathways for treatment of cancer.

### BRIEF SUMMARY OF THE INVENTION

In one aspect, the present invention provides heteroaryl compounds of Formula (I) or stereoisomers or pharmaceutically acceptable salts, esters, or prodrugs thereof:

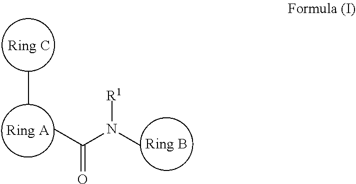

wherein
R¹ is H, alkyl, cycloalkyl, or substituted alkyl;
ring A is a 5 or 6-membered aryl or heteroaryl; wherein the 5 or 6-membered aryl or heteroaryl is optionally substituted with one or more substituents selected from alkyl, halo, or haloalkyl; wherein the 5 or 6-membered heteroaryl each has at least one ring-forming carbon atom and 1, 2 or 3 ring-forming heteroatoms independently selected from N, O, and S; wherein the N is optionally substituted by oxo to form N⁺—O⁻; wherein a ring-forming carbon atom of the 5 or 6-membered aryl or heteroaryl is optionally substituted by oxo to form a carbonyl group;
ring B is a 5 or 6-membered monocyclic aryl or heteroaryl, or 8- to 12-membered fused bicyclic aryl or heteroaryl; wherein the monocyclic or fused bicyclic aryl or heteroaryl is optionally substituted with 1-5 substituents, each of which is independently alkyl, haloalkyl, halo, hydroxy, hydroxyalkyl, alkoxy, alkoxyalkyl, alkthioalkyl, alk-S(═O)₂-alkyl, cyano, hydroxyalkamino, amino, amido, alkamino, —NR¹⁰R¹¹, cycloalkyl, alk-cycloalkyl, heterocycloalkyl, alk-heterocycloalkyl, aryl, heteroaryl, bridged bicyclic cycloalkyl or heterocyloalkyl, fused bicyclic cycloalkyl or heterocyloalkyl, spirocyclic cycloalkyl or heterocyloalkyl, or multicycloalkyl; wherein each substituent is optionally further substituted; wherein a ring-forming carbon atom of the cycloalkyl or heterocycloalkyl is optionally substituted by oxo to form a carbonyl group; wherein R¹⁰ and R¹¹ are each independently selected from H, alkyl, halo, haloalkyl, cycloalkyl, alk-cycloalkyl, alk-heterocycloalkyl, or heterocycloalkyl, each of which is optionally further substituted;
ring C is a 5- or 6-membered aryl or heteroaryl, optionally substituted with one or more substituents, each of which is independently selected from alkyl, haloalkyl, halo, hydroxy, hydroxyalkyl, hydroxyalkamino, alkoxy, alkoxyalkyl, amino, alkylamino, dialkylamino, amido, alkamino, cyano, heterocycloalkyl, cycloalkyl, alk-cycloalkyl or alk-heterocycloalkyl;
wherein the heteroaryl each has at least one ring-forming carbon atom and 1, 2 or 3 ring-forming heteroatoms independently selected from N, O, and S;
wherein any hydrogen (H) can be optionally replaced by deuterium (D).

In some embodiments, R¹ is H.

In some embodiments, ring A is

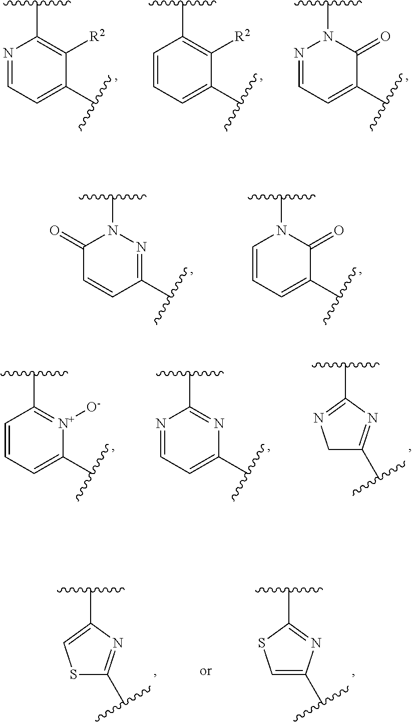

in which R² is H, alkyl, halo, or haloalkyl. In some preferred embodiments, R² is halo (e.g., F).

In some embodiments, ring B in Formula (I) is

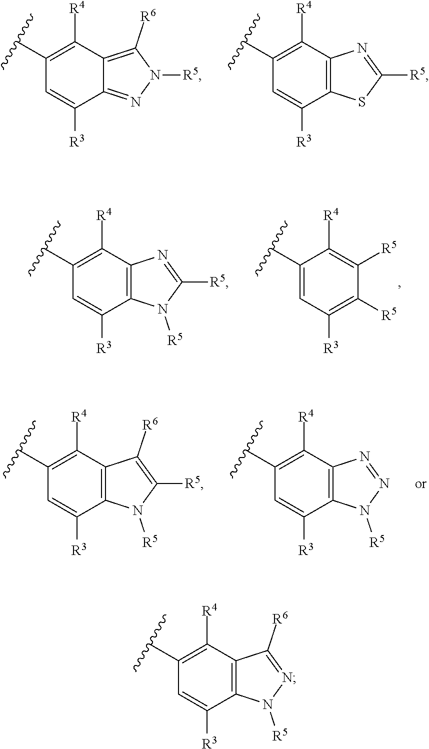

in which, R³ is H, halo, haloalkyl or alkyl; preferably, R³ is H or halo (e.g., F);
R⁴ is cycloalkyl, heterocycloalkyl, bridged bicyclic cycloalkyl or heterocyloalkyl, fused bicyclic cycloalkyl or heterocyloalkyl, spirocyclic cycloalkyl or heterocyloalkyl, multicycloalkyl, or —NR¹⁰R¹¹; wherein each substituent is optionally further substituted; wherein R¹⁰ and R¹¹ each are independently selected from H, halo, haloalkyl, alkyl, cycloalkyl, alk-cycloalkyl, alk-heterocycloalkyl, or heterocycloalkyl;
each R⁵ is independently H, alkyl, CD₃, alkthioalkyl, alk-S(═O)₂-alkyl, cyano, amino, hydroxyl, hydroxyalkyl, hydroxyalkamino, amido, alkamino, halo, haloalkyl, alkoxy, alkoxyalkyl, cycloalkyl, heterocyloalkyl, fused cycloalkyl, bridged cycloalkyl, spirocycloalkyl, multicycloalkyl, aryl, or heteroaryl, wherein each substituent is optionally further substituted; wherein any hydrogen of alkyl can be optionally replaced with D; and
R⁶ is H, halo, haloalkyl, or alkyl.

In some embodiments, R³ is H or F.

In some embodiments, R⁶ is H or F.

In some embodiments, R⁴ is cycloalkyl, heterocycloalkyl, bridged bicyclic cycloalkyl or heterocyloalkyl, fused bicyclic cycloalkyl or heterocyloalkyl, spirocyclic cycloalkyl or heterocyloalkyl, or multicycloalkyl, each of which is optionally further substituted with one or more substituents selected from hydroxyl, hydroxyalkyl, hydroxyalkamino, halo, haloalkyl, alkyl, amino, alkylamino, dialkylamino, alkamino, alkoxy, alkoxyalkyl, —NH—C(═O)—(CH₂)ₙ—NH₂, amido, cycloalkyl, alk-cycloalkyl, alk-heterocycloalkyl, or heterocycloalkyl; n is 0, 1, 2 or 3.

In some embodiments, ring C in formula (I) is phenyl, substituted with one or more substituents, each of which is independently selected from —F, —Cl, or alkoxy. In some preferred embodiments, alkoxy is —O—CH₃.

Specific example of ring C in Formula (I) is

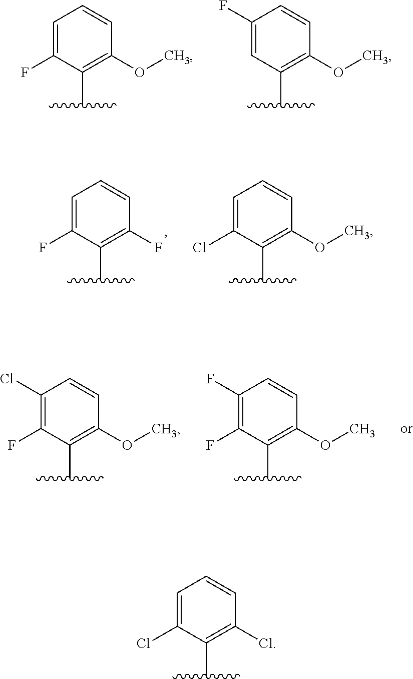

In some embodiments, the compound is of Formula (Ia) or Formula (Ib), or a stereoisomer, or pharmaceutically acceptable salt thereof,

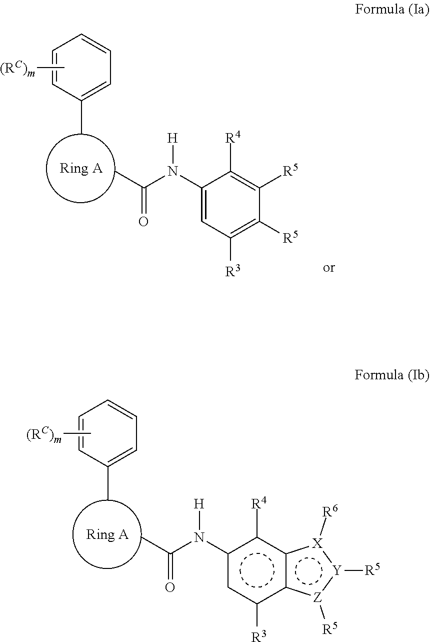

wherein
ring A is

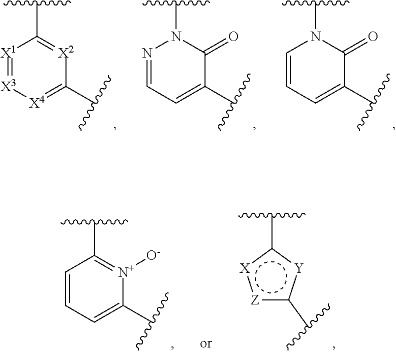

and ring A is optionally further substituted with one or more R²;
X¹, X², X³, and X⁴ are each independently a carbon atom (C) or N, and optionally substituted with R²;
R² is H, alkyl, halo or haloalkyl;
each R^{c} is independently alkyl, haloalkyl, halo, hydroxy, hydroxyalkyl, hydroxyalkamino, alkoxy, alkoxyalkyl, amino, alkylamino, dialkylamino, amido, alkamino, cyano, cycloalkyl, alk-cycloalkyl, or heterocycloalkyl;
m is 0, 1, 2, 3 or 4;
X, Y or Z each is independently a carbon atom or heteroatom selected from N or S;
dashed bond represents a single bond or a double bond as required to complete the valencies of the atoms being linked by the bond;
R³, R⁴, R⁵, and R⁶ each are the same as described above.

Examples of compounds of Formula (I) include:

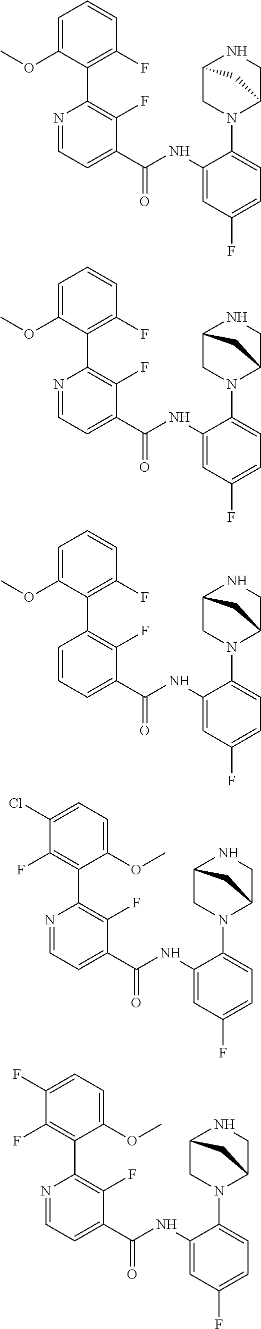 [SMILES: COc1cccc(F)c1-c1nccc(C(=O)Nc2cc(F)ccc2N2C[C@@H]3C[C@H]2CN3)c1F ; COc1cccc(F)c1-c1nccc(C(=O)Nc2cc(F)ccc2N2C[C@H]3C[C@@H]2CN3)c1F ; COc1cccc(F)c1-c1cccc(C(=O)Nc2cc(F)ccc2N2C[C@H]3C[C@@H]2CN3)c1F ; COc1ccc(Cl)c(F)c1-c1nccc(C(=O)Nc2cc(F)ccc2N2C[C@H]3C[C@@H]2CN3)c1F ; COc1ccc(F)c(F)c1-c1nccc(C(=O)Nc2cc(F)ccc2N2C[C@H]3C[C@@H]2CN3)c1F]

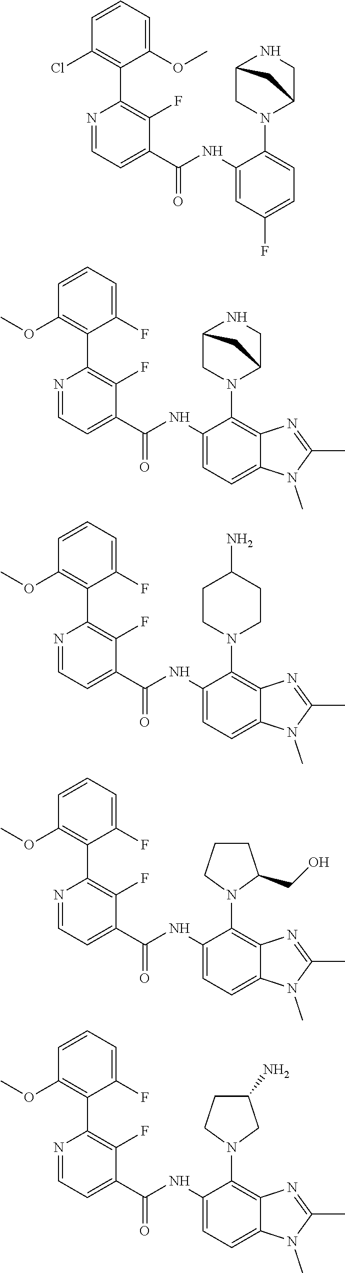

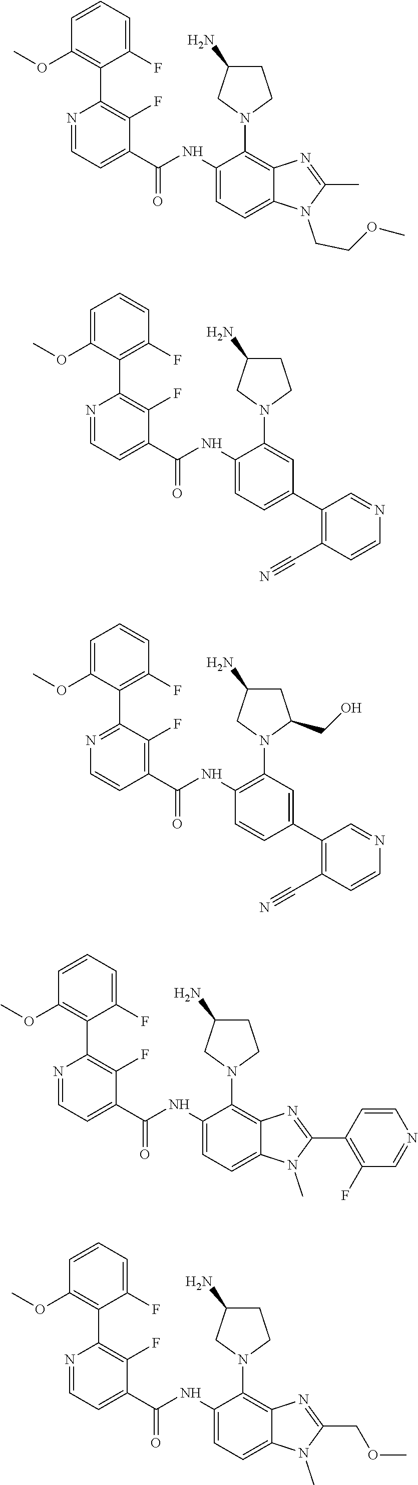

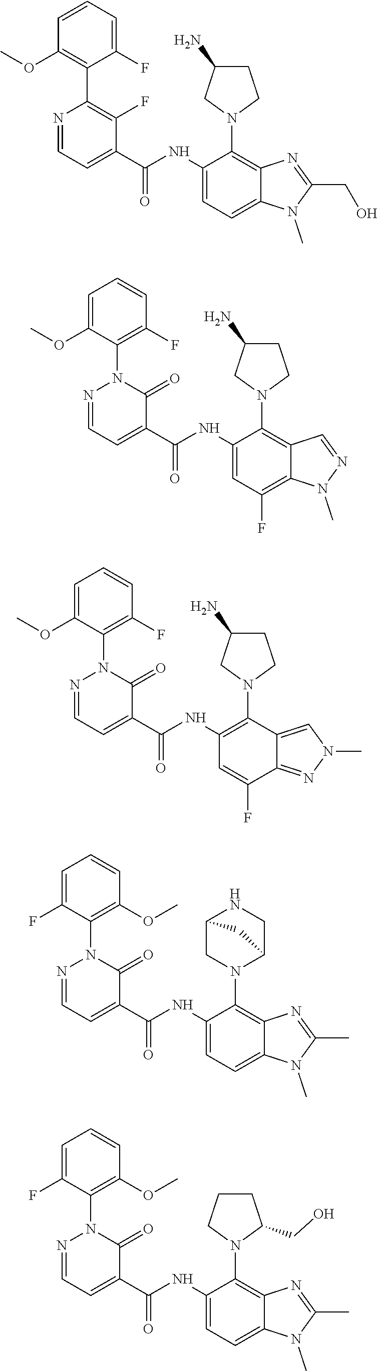

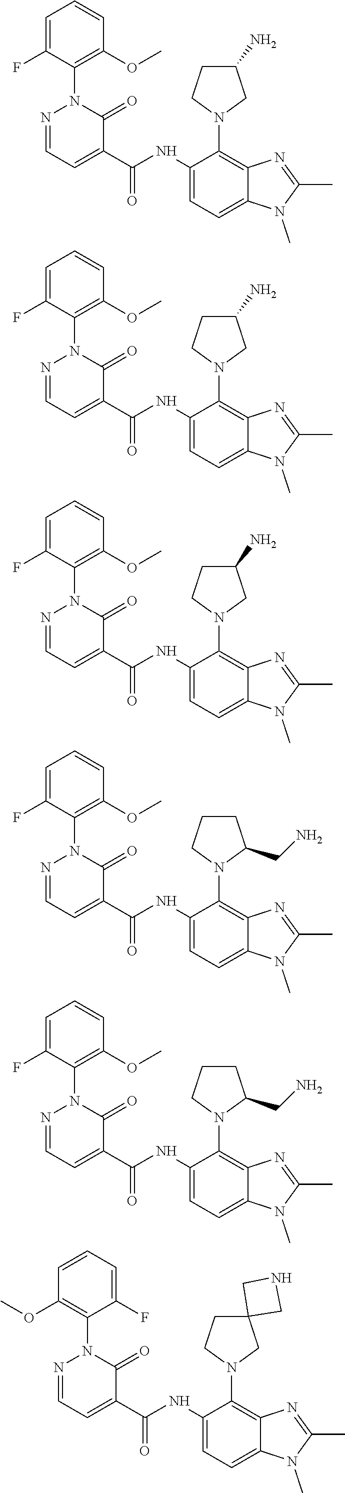

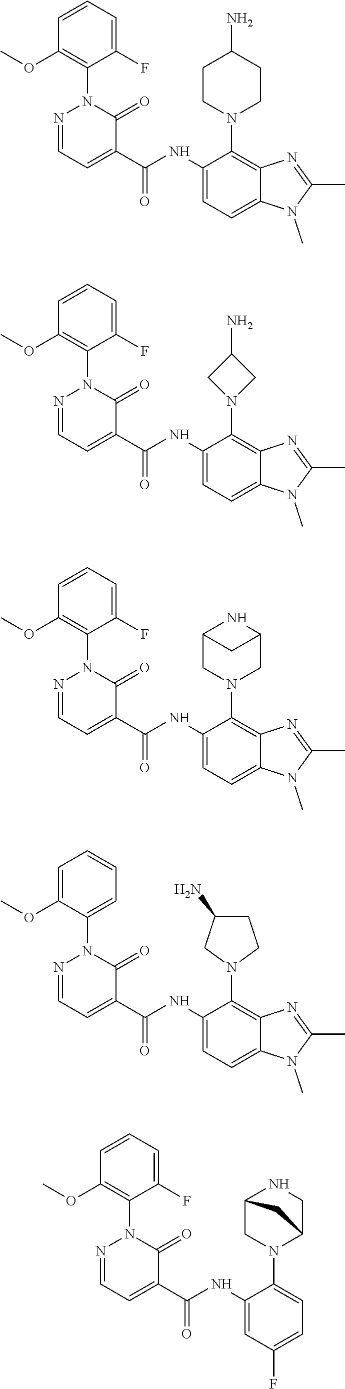

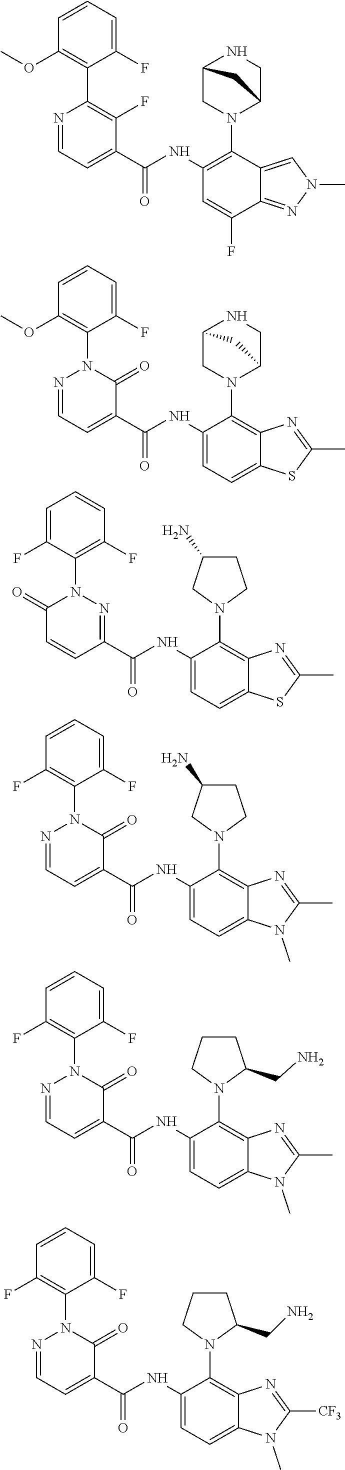

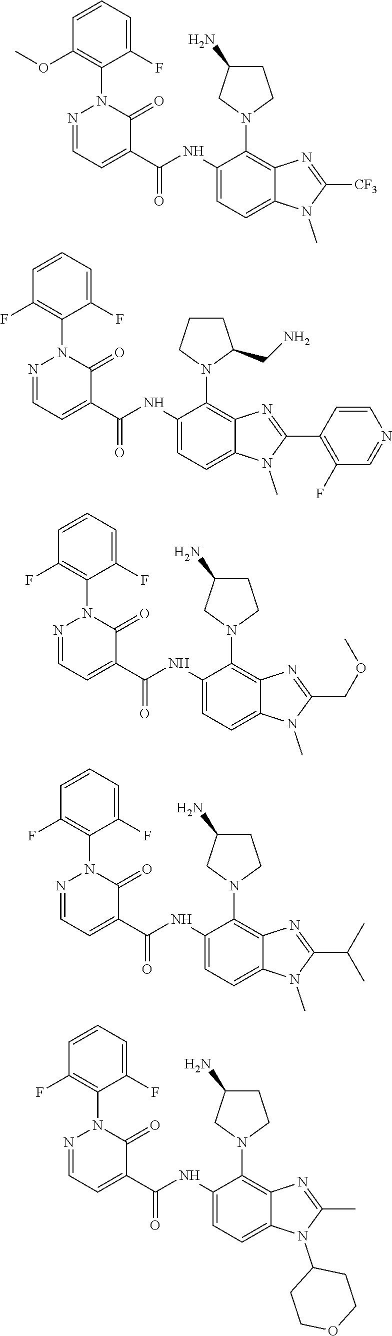 [SMILES: COc1cccc(F)c1-n1nccc(C(=O)Nc2ccc3c(nc(C(F)(F)F)n3C)c2N2CC[C@H](N)C2)c1=O ; Cn1c(-c2ccncc2F)nc2c(N3CCC[C@H]3CN)c(NC(=O)c3ccnn(-c4c(F)cccc4F)c3=O)ccc21 ; COCc1nc2c(N3CC[C@H](N)C3)c(NC(=O)c3ccnn(-c4c(F)cccc4F)c3=O)ccc2n1C ; CC(C)c1nc2c(N3CC[C@H](N)C3)c(NC(=O)c3ccnn(-c4c(F)cccc4F)c3=O)ccc2n1C ; Cc1nc2c(N3CC[C@H](N)C3)c(NC(=O)c3ccnn(-c4c(F)cccc4F)c3=O)ccc2n1C1CCOCC1]

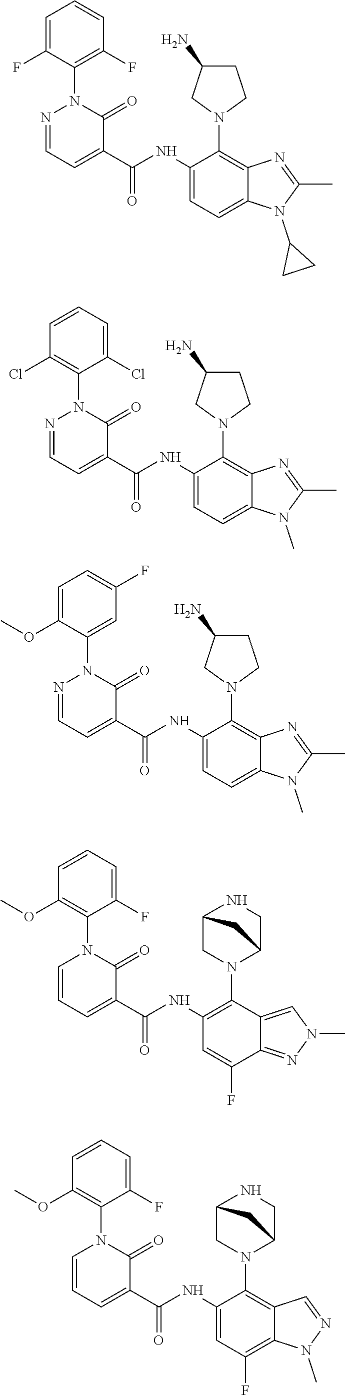

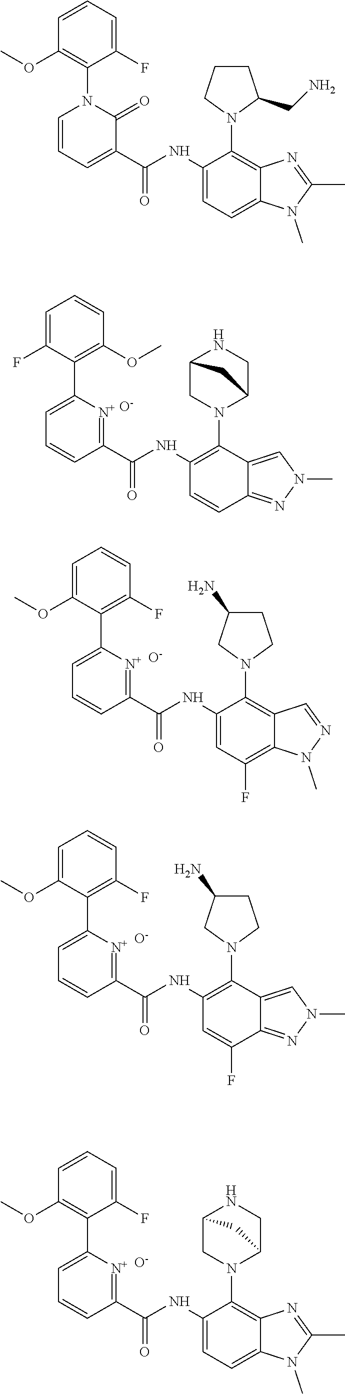

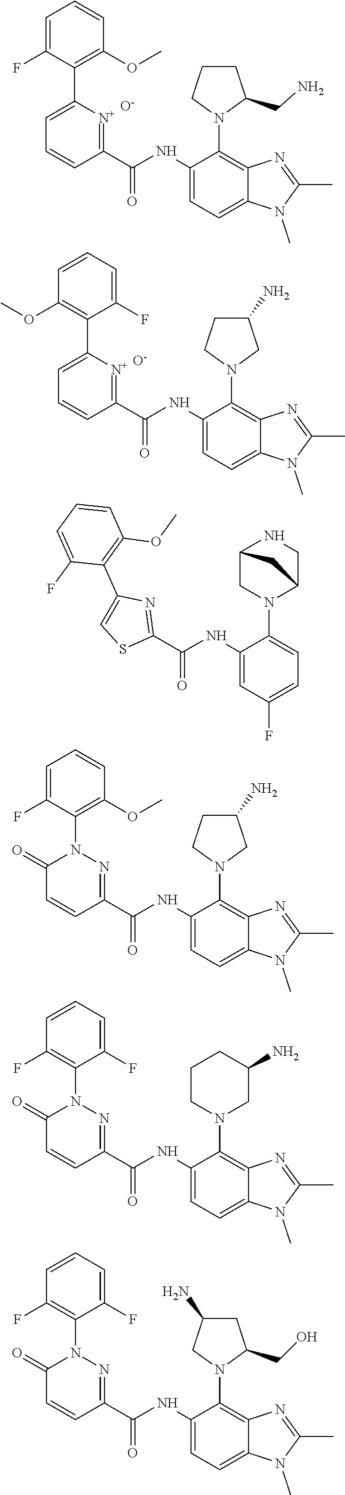

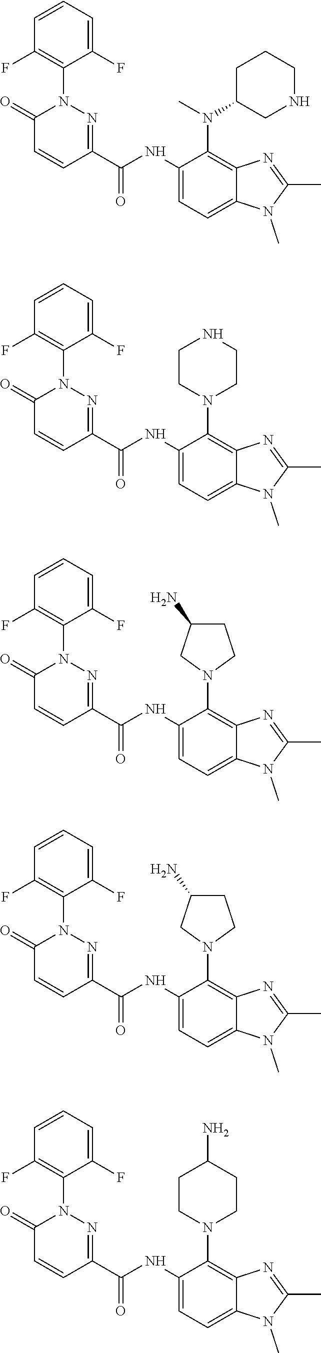 [SMILES: Cc1nc2c(N(C)[C@@H]3CCCNC3)c(NC(=O)c3ccc(=O)n(-c4c(F)cccc4F)n3)ccc2n1C ; Cc1nc2c(N3CCNCC3)c(NC(=O)c3ccc(=O)n(-c4c(F)cccc4F)n3)ccc2n1C ; Cc1nc2c(N3CC[C@H](N)C3)c(NC(=O)c3ccc(=O)n(-c4c(F)cccc4F)n3)ccc2n1C ; Cc1nc2c(N3CC[C@@H](N)C3)c(NC(=O)c3ccc(=O)n(-c4c(F)cccc4F)n3)ccc2n1C ; Cc1nc2c(N3CCC(N)CC3)c(NC(=O)c3ccc(=O)n(-c4c(F)cccc4F)n3)ccc2n1C]

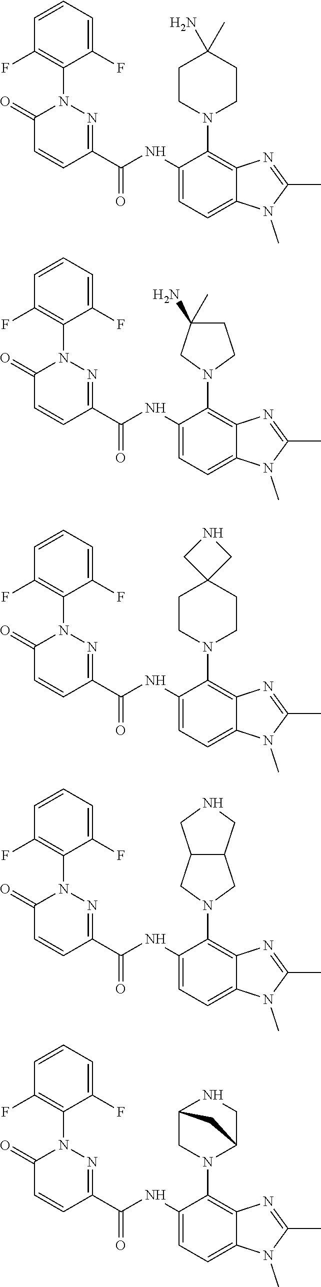 [SMILES: Cc1nc2c(N3CCC(C)(N)CC3)c(NC(=O)c3ccc(=O)n(-c4c(F)cccc4F)n3)ccc2n1C ; Cc1nc2c(N3CC[C@](C)(N)C3)c(NC(=O)c3ccc(=O)n(-c4c(F)cccc4F)n3)ccc2n1C ; Cc1nc2c(N3CCC4(CC3)CNC4)c(NC(=O)c3ccc(=O)n(-c4c(F)cccc4F)n3)ccc2n1C ; Cc1nc2c(N3CC4CNCC4C3)c(NC(=O)c3ccc(=O)n(-c4c(F)cccc4F)n3)ccc2n1C ; Cc1nc2c(N3C[C@H]4C[C@@H]3CN4)c(NC(=O)c3ccc(=O)n(-c4c(F)cccc4F)n3)ccc2n1C]

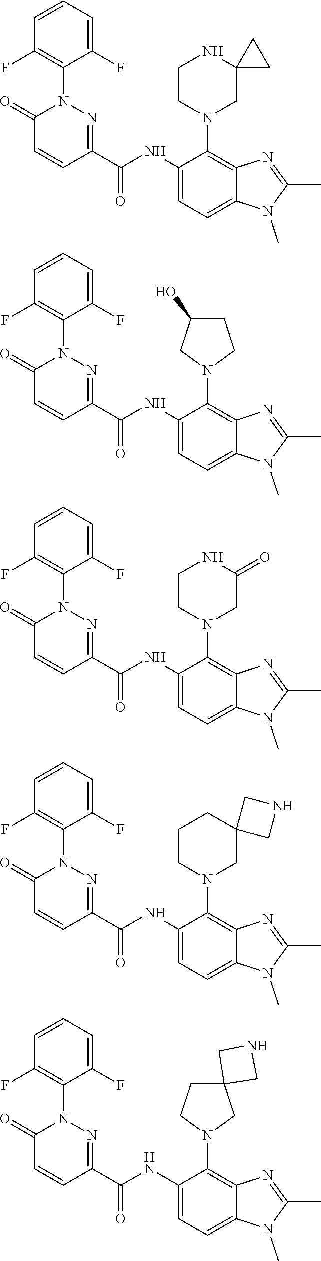 [SMILES: Cc1nc2c(N3CCNC4(CC4)C3)c(NC(=O)c3ccc(=O)n(-c4c(F)cccc4F)n3)ccc2n1C ; Cc1nc2c(N3CC[C@H](O)C3)c(NC(=O)c3ccc(=O)n(-c4c(F)cccc4F)n3)ccc2n1C ; Cc1nc2c(N3CCNC(=O)C3)c(NC(=O)c3ccc(=O)n(-c4c(F)cccc4F)n3)ccc2n1C ; Cc1nc2c(N3CCCC4(CNC4)C3)c(NC(=O)c3ccc(=O)n(-c4c(F)cccc4F)n3)ccc2n1C ; Cc1nc2c(N3CCC4(CNC4)C3)c(NC(=O)c3ccc(=O)n(-c4c(F)cccc4F)n3)ccc2n1C]

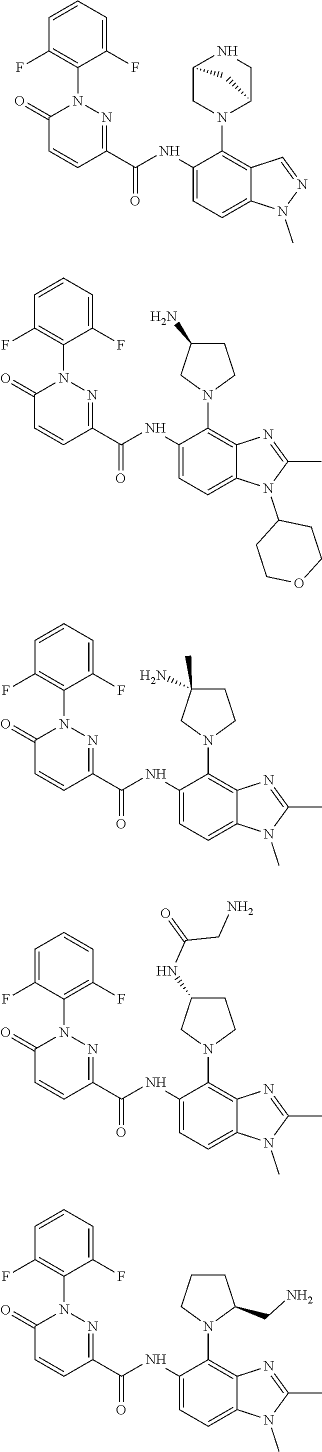 [SMILES: Cn1ncc2c(N3C[C@@H]4C[C@H]3CN4)c(NC(=O)c3ccc(=O)n(-c4c(F)cccc4F)n3)ccc21 ; Cc1nc2c(N3CC[C@H](N)C3)c(NC(=O)c3ccc(=O)n(-c4c(F)cccc4F)n3)ccc2n1C1CCOCC1 ; Cc1nc2c(N3CC[C@@](C)(N)C3)c(NC(=O)c3ccc(=O)n(-c4c(F)cccc4F)n3)ccc2n1C ; Cc1nc2c(N3CC[C@@H](NC(=O)CN)C3)c(NC(=O)c3ccc(=O)n(-c4c(F)cccc4F)n3)ccc2n1C ; Cc1nc2c(N3CCC[C@H]3CN)c(NC(=O)c3ccc(=O)n(-c4c(F)cccc4F)n3)ccc2n1C]

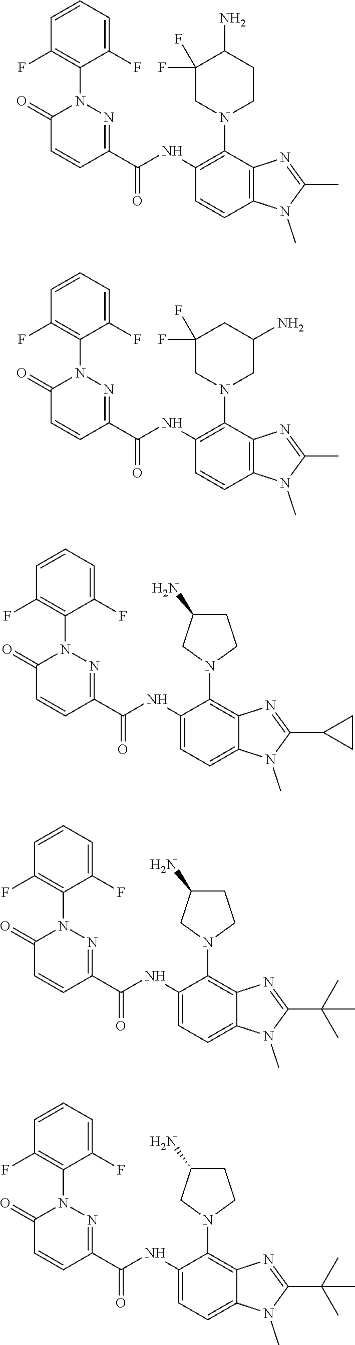 [SMILES: Cc1nc2c(N3CCC(N)C(F)(F)C3)c(NC(=O)c3ccc(=O)n(-c4c(F)cccc4F)n3)ccc2n1C ; Cc1nc2c(N3CC(N)CC(F)(F)C3)c(NC(=O)c3ccc(=O)n(-c4c(F)cccc4F)n3)ccc2n1C ; Cn1c(C2CC2)nc2c(N3CC[C@H](N)C3)c(NC(=O)c3ccc(=O)n(-c4c(F)cccc4F)n3)ccc21 ; Cn1c(C(C)(C)C)nc2c(N3CC[C@H](N)C3)c(NC(=O)c3ccc(=O)n(-c4c(F)cccc4F)n3)ccc21 ; Cn1c(C(C)(C)C)nc2c(N3CC[C@@H](N)C3)c(NC(=O)c3ccc(=O)n(-c4c(F)cccc4F)n3)ccc21]

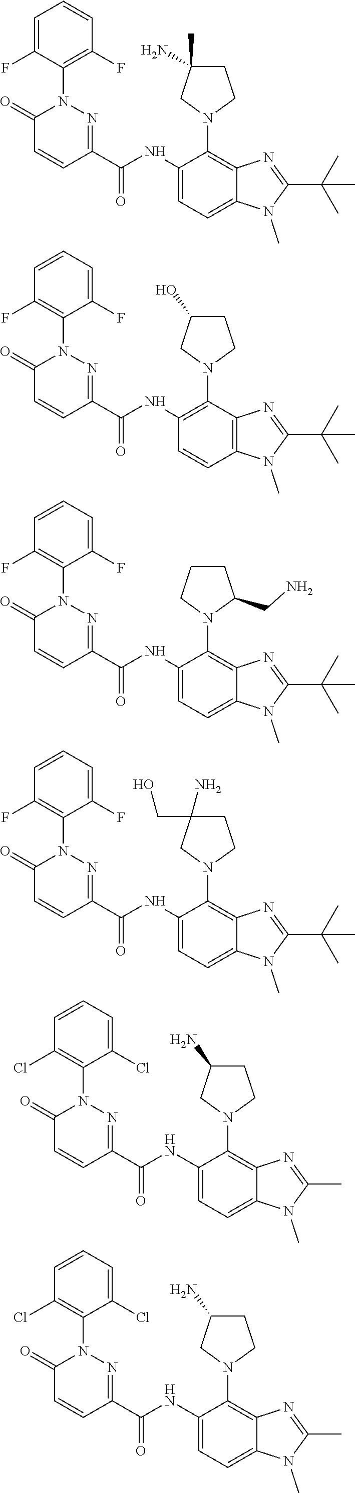 [SMILES: Cn1c(C(C)(C)C)nc2c(N3CC[C@@](C)(N)C3)c(NC(=O)c3ccc(=O)n(-c4c(F)cccc4F)n3)ccc21 ; Cn1c(C(C)(C)C)nc2c(N3CC[C@@H](O)C3)c(NC(=O)c3ccc(=O)n(-c4c(F)cccc4F)n3)ccc21 ; Cn1c(C(C)(C)C)nc2c(N3CCC[C@H]3CN)c(NC(=O)c3ccc(=O)n(-c4c(F)cccc4F)n3)ccc21 ; Cn1c(C(C)(C)C)nc2c(N3CCC(N)(CO)C3)c(NC(=O)c3ccc(=O)n(-c4c(F)cccc4F)n3)ccc21 ; Cc1nc2c(N3CC[C@H](N)C3)c(NC(=O)c3ccc(=O)n(-c4c(Cl)cccc4Cl)n3)ccc2n1C ; Cc1nc2c(N3CC[C@@H](N)C3)c(NC(=O)c3ccc(=O)n(-c4c(Cl)cccc4Cl)n3)ccc2n1C]

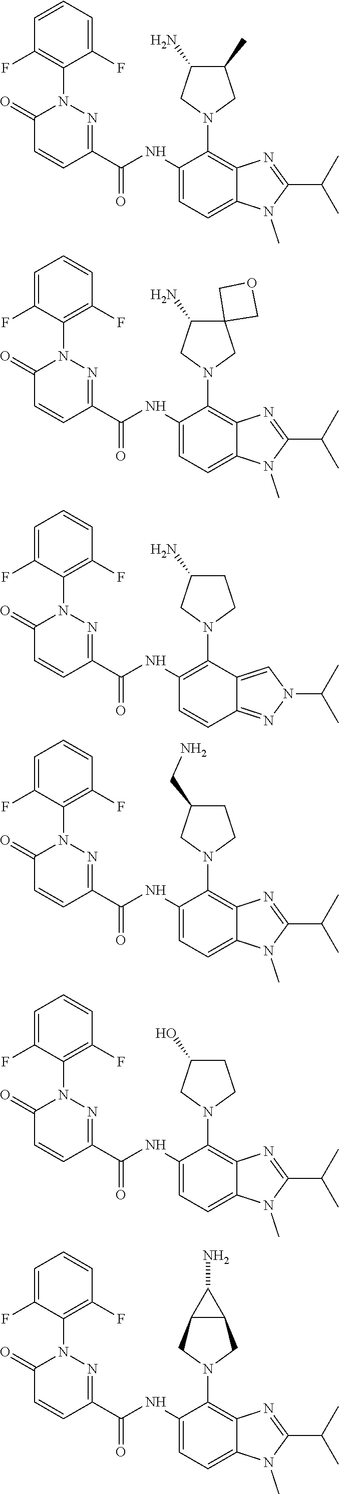

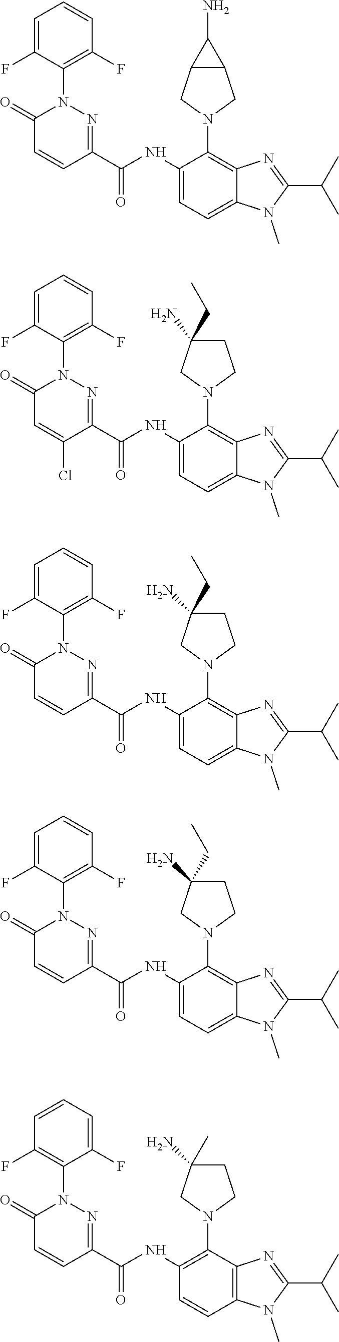 [SMILES: CC(C)c1nc2c(N3CC4C(N)C4C3)c(NC(=O)c3ccc(=O)n(-c4c(F)cccc4F)n3)ccc2n1C ; CC[C@@]1(N)CCN(c2c(NC(=O)c3nn(-c4c(F)cccc4F)c(=O)cc3Cl)ccc3c2nc(C(C)C)n3C)C1 ; CC[C@@]1(N)CCN(c2c(NC(=O)c3ccc(=O)n(-c4c(F)cccc4F)n3)ccc3c2nc(C(C)C)n3C)C1 ; CC[C@]1(N)CCN(c2c(NC(=O)c3ccc(=O)n(-c4c(F)cccc4F)n3)ccc3c2nc(C(C)C)n3C)C1 ; CC(C)c1nc2c(N3CC[C@@](C)(N)C3)c(NC(=O)c3ccc(=O)n(-c4c(F)cccc4F)n3)ccc2n1C]

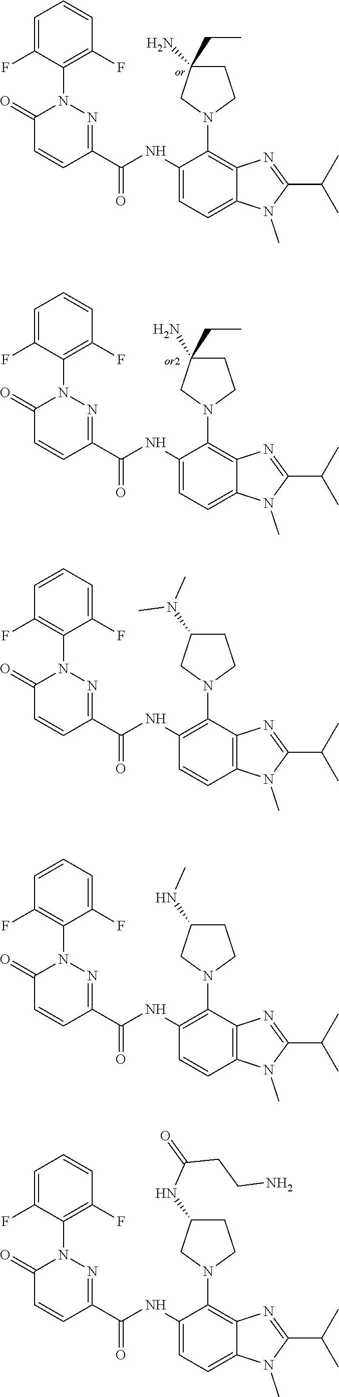 [SMILES: CC[C@@]1(N)CCN(c2c(NC(=O)c3ccc(=O)n(-c4c(F)cccc4F)n3)ccc3c2nc(C(C)C)n3C)C1 ; CC[C@@]1(N)CCN(c2c(NC(=O)c3ccc(=O)n(-c4c(F)cccc4F)n3)ccc3c2nc(C(C)C)n3C)C1 ; CC(C)c1nc2c(N3CC[C@@H](N(C)C)C3)c(NC(=O)c3ccc(=O)n(-c4c(F)cccc4F)n3)ccc2n1C ; CN[C@@H]1CCN(c2c(NC(=O)c3ccc(=O)n(-c4c(F)cccc4F)n3)ccc3c2nc(C(C)C)n3C)C1 ; CC(C)c1nc2c(N3CC[C@@H](NC(=O)CCN)C3)c(NC(=O)c3ccc(=O)n(-c4c(F)cccc4F)n3)ccc2n1C]

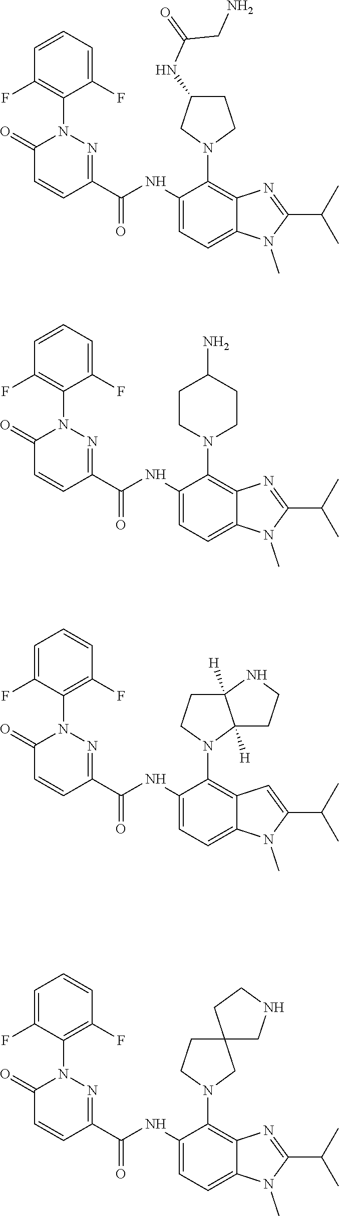 [SMILES: CC(C)c1nc2c(N3CC[C@@H](NC(=O)CN)C3)c(NC(=O)c3ccc(=O)n(-c4c(F)cccc4F)n3)ccc2n1C ; CC(C)c1nc2c(N3CCC(N)CC3)c(NC(=O)c3ccc(=O)n(-c4c(F)cccc4F)n3)ccc2n1C ; CC(C)c1cc2c(N3CC[C@H]4NCC[C@H]43)c(NC(=O)c3ccc(=O)n(-c4c(F)cccc4F)n3)ccc2n1C ; CC(C)c1nc2c(N3CCC4(CCNC4)C3)c(NC(=O)c3ccc(=O)n(-c4c(F)cccc4F)n3)ccc2n1C]

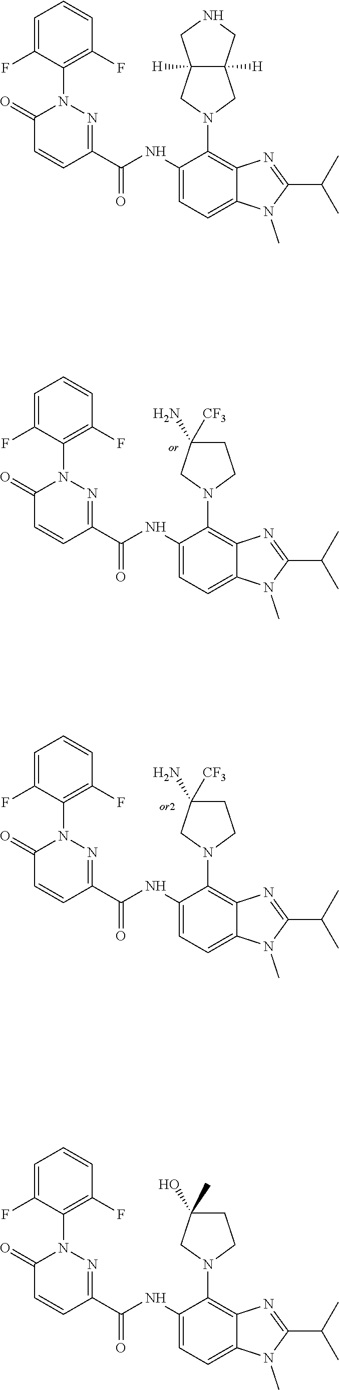 [SMILES: CC(C)c1nc2c(N3C[C@H]4CNC[C@H]4C3)c(NC(=O)c3ccc(=O)n(-c4c(F)cccc4F)n3)ccc2n1C ; CC(C)c1nc2c(N3CC[C@](N)(C(F)(F)F)C3)c(NC(=O)c3ccc(=O)n(-c4c(F)cccc4F)n3)ccc2n1C ; CC(C)c1nc2c(N3CC[C@](N)(C(F)(F)F)C3)c(NC(=O)c3ccc(=O)n(-c4c(F)cccc4F)n3)ccc2n1C ; CC(C)c1nc2c(N3CC[C@@](C)(O)C3)c(NC(=O)c3ccc(=O)n(-c4c(F)cccc4F)n3)ccc2n1C]

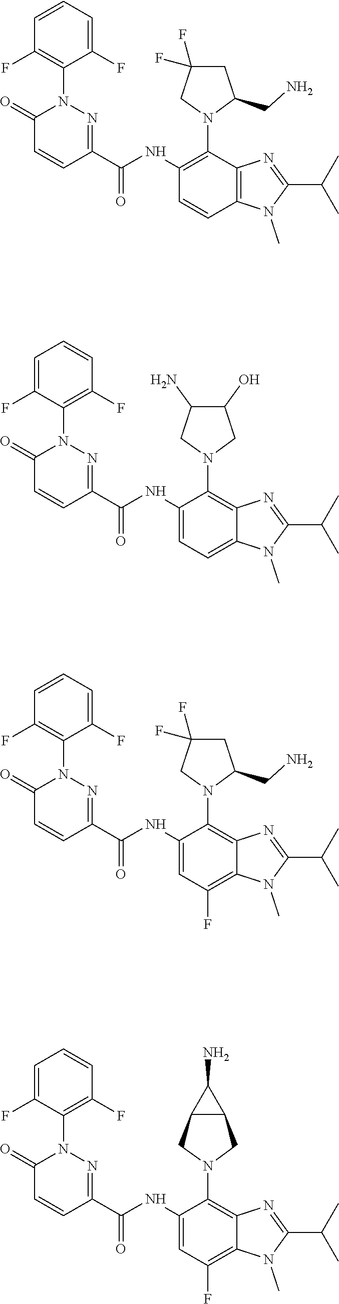 [SMILES: CC(C)c1nc2c(N3CC(F)(F)C[C@H]3CN)c(NC(=O)c3ccc(=O)n(-c4c(F)cccc4F)n3)ccc2n1C ; CC(C)c1nc2c(N3CC(N)C(O)C3)c(NC(=O)c3ccc(=O)n(-c4c(F)cccc4F)n3)ccc2n1C ; CC(C)c1nc2c(N3CC(F)(F)C[C@H]3CN)c(NC(=O)c3ccc(=O)n(-c4c(F)cccc4F)n3)cc(F)c2n1C ; CC(C)c1nc2c(N3C[C@@H]4[C@H](N)[C@@H]4C3)c(NC(=O)c3ccc(=O)n(-c4c(F)cccc4F)n3)cc(F)c2n1C]

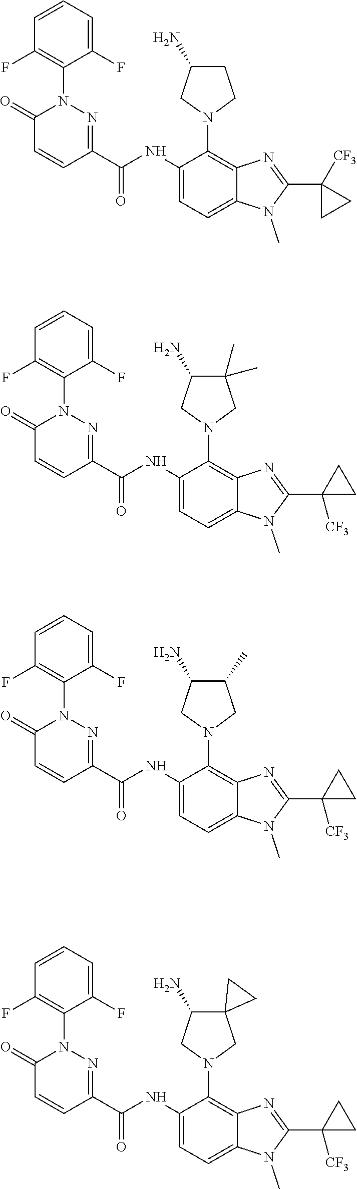 [SMILES: Cn1c(C2(C(F)(F)F)CC2)nc2c(N3CC[C@@H](N)C3)c(NC(=O)c3ccc(=O)n(-c4c(F)cccc4F)n3)ccc21 ; Cn1c(C2(C(F)(F)F)CC2)nc2c(N3C[C@H](N)C(C)(C)C3)c(NC(=O)c3ccc(=O)n(-c4c(F)cccc4F)n3)ccc21 ; C[C@@H]1CN(c2c(NC(=O)c3ccc(=O)n(-c4c(F)cccc4F)n3)ccc3c2nc(C2(C(F)(F)F)CC2)n3C)C[C@@H]1N ; Cn1c(C2(C(F)(F)F)CC2)nc2c(N3C[C@H](N)C4(CC4)C3)c(NC(=O)c3ccc(=O)n(-c4c(F)cccc4F)n3)ccc21]

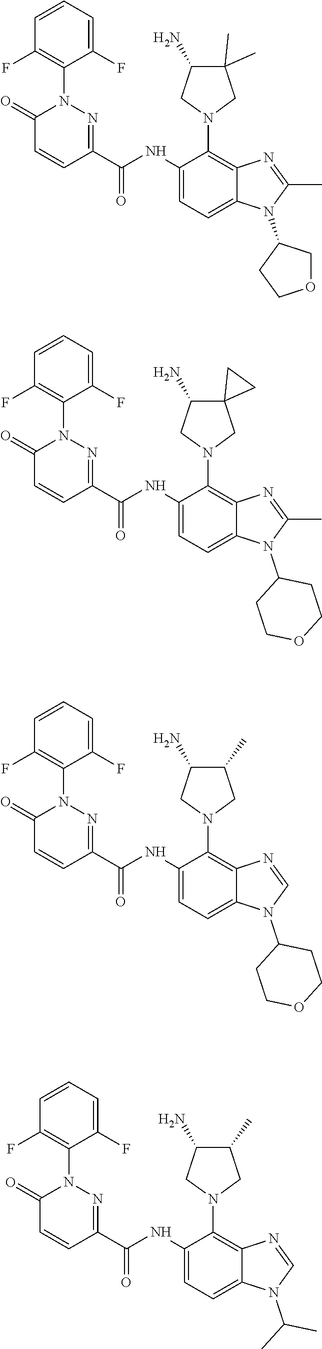 [SMILES: Cc1nc2c(N3C[C@H](N)C(C)(C)C3)c(NC(=O)c3ccc(=O)n(-c4c(F)cccc4F)n3)ccc2n1[C@H]1CCOC1 ; Cc1nc2c(N3C[C@H](N)C4(CC4)C3)c(NC(=O)c3ccc(=O)n(-c4c(F)cccc4F)n3)ccc2n1C1CCOCC1 ; C[C@@H]1CN(c2c(NC(=O)c3ccc(=O)n(-c4c(F)cccc4F)n3)ccc3c2ncn3C2CCOCC2)C[C@@H]1N ; CC(C)n1cnc2c(N3C[C@@H](C)[C@@H](N)C3)c(NC(=O)c3ccc(=O)n(-c4c(F)cccc4F)n3)ccc21]

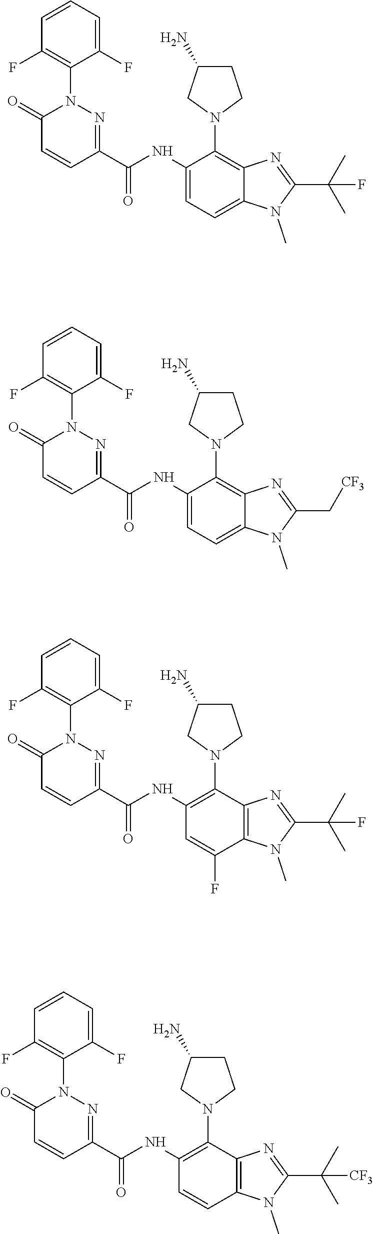 [SMILES: Cn1c(C(C)(C)F)nc2c(N3CC[C@@H](N)C3)c(NC(=O)c3ccc(=O)n(-c4c(F)cccc4F)n3)ccc21 ; Cn1c(CC(F)(F)F)nc2c(N3CC[C@@H](N)C3)c(NC(=O)c3ccc(=O)n(-c4c(F)cccc4F)n3)ccc21 ; Cn1c(C(C)(C)F)nc2c(N3CC[C@@H](N)C3)c(NC(=O)c3ccc(=O)n(-c4c(F)cccc4F)n3)cc(F)c21 ; Cn1c(C(C)(C)C(F)(F)F)nc2c(N3CC[C@@H](N)C3)c(NC(=O)c3ccc(=O)n(-c4c(F)cccc4F)n3)ccc21]

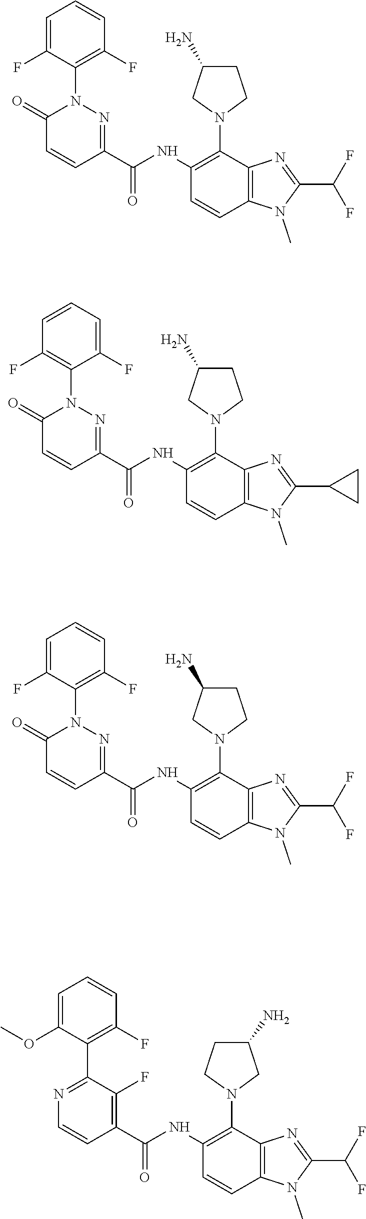 [SMILES: Cn1c(C(F)F)nc2c(N3CC[C@@H](N)C3)c(NC(=O)c3ccc(=O)n(-c4c(F)cccc4F)n3)ccc21 ; Cn1c(C2CC2)nc2c(N3CC[C@@H](N)C3)c(NC(=O)c3ccc(=O)n(-c4c(F)cccc4F)n3)ccc21 ; Cn1c(C(F)F)nc2c(N3CC[C@H](N)C3)c(NC(=O)c3ccc(=O)n(-c4c(F)cccc4F)n3)ccc21 ; COc1cccc(F)c1-c1nccc(C(=O)Nc2ccc3c(nc(C(F)F)n3C)c2N2CC[C@H](N)C2)c1F]

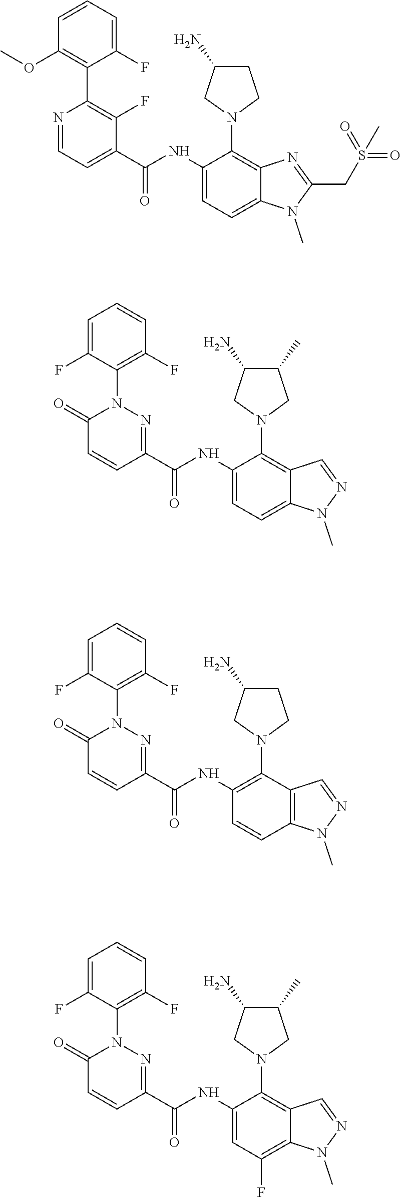 [SMILES: COc1cccc(F)c1-c1nccc(C(=O)Nc2ccc3c(nc(CS(C)(=O)=O)n3C)c2N2CC[C@@H](N)C2)c1F ; C[C@@H]1CN(c2c(NC(=O)c3ccc(=O)n(-c4c(F)cccc4F)n3)ccc3c2cnn3C)C[C@@H]1N ; Cn1ncc2c(N3CC[C@@H](N)C3)c(NC(=O)c3ccc(=O)n(-c4c(F)cccc4F)n3)ccc21 ; C[C@@H]1CN(c2c(NC(=O)c3ccc(=O)n(-c4c(F)cccc4F)n3)cc(F)c3c2cnn3C)C[C@@H]1N]

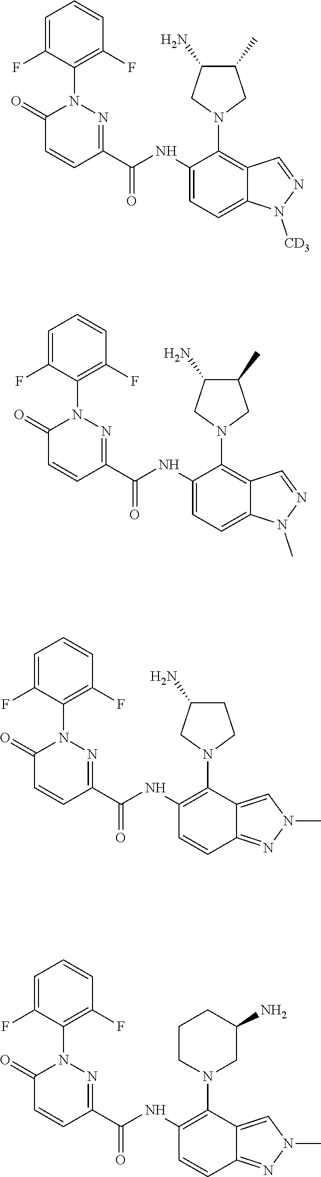 [SMILES: [2H]C([2H])([2H])n1ncc2c(N3C[C@@H](C)[C@@H](N)C3)c(NC(=O)c3ccc(=O)n(-c4c(F)cccc4F)n3)ccc21 ; C[C@H]1CN(c2c(NC(=O)c3ccc(=O)n(-c4c(F)cccc4F)n3)ccc3c2cnn3C)C[C@@H]1N ; Cn1cc2c(N3CC[C@@H](N)C3)c(NC(=O)c3ccc(=O)n(-c4c(F)cccc4F)n3)ccc2n1 ; Cn1cc2c(N3CCC[C@@H](N)C3)c(NC(=O)c3ccc(=O)n(-c4c(F)cccc4F)n3)ccc2n1]

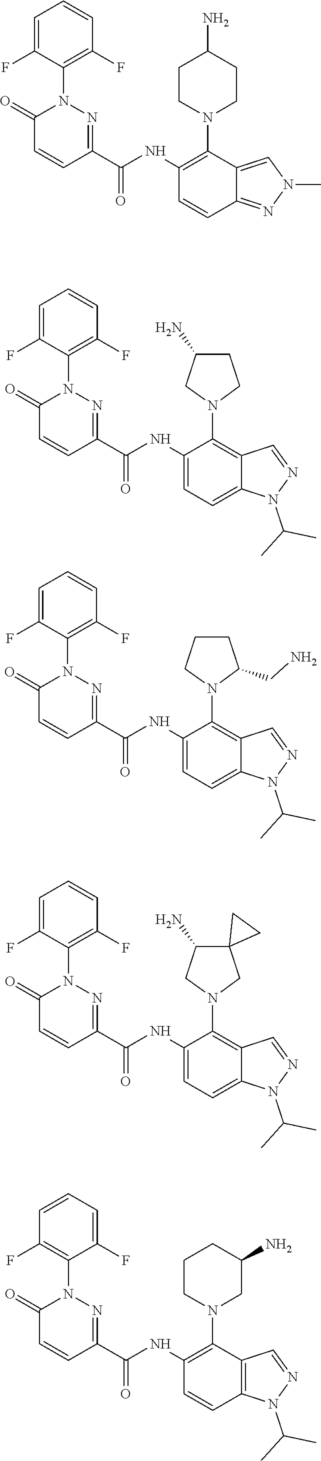 [SMILES: Cn1cc2c(N3CCC(N)CC3)c(NC(=O)c3ccc(=O)n(-c4c(F)cccc4F)n3)ccc2n1 ; CC(C)n1ncc2c(N3CC[C@@H](N)C3)c(NC(=O)c3ccc(=O)n(-c4c(F)cccc4F)n3)ccc21 ; CC(C)n1ncc2c(N3CCC[C@@H]3CN)c(NC(=O)c3ccc(=O)n(-c4c(F)cccc4F)n3)ccc21 ; CC(C)n1ncc2c(N3C[C@H](N)C4(CC4)C3)c(NC(=O)c3ccc(=O)n(-c4c(F)cccc4F)n3)ccc21 ; CC(C)n1ncc2c(N3CCC[C@@H](N)C3)c(NC(=O)c3ccc(=O)n(-c4c(F)cccc4F)n3)ccc21]

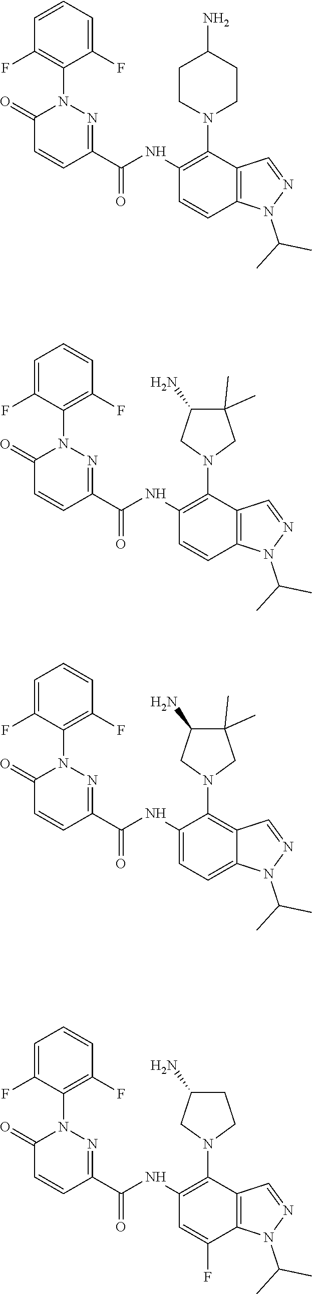 [SMILES: CC(C)n1ncc2c(N3CCC(N)CC3)c(NC(=O)c3ccc(=O)n(-c4c(F)cccc4F)n3)ccc21 ; CC(C)n1ncc2c(N3C[C@H](N)C(C)(C)C3)c(NC(=O)c3ccc(=O)n(-c4c(F)cccc4F)n3)ccc21 ; CC(C)n1ncc2c(N3C[C@@H](N)C(C)(C)C3)c(NC(=O)c3ccc(=O)n(-c4c(F)cccc4F)n3)ccc21 ; CC(C)n1ncc2c(N3CC[C@@H](N)C3)c(NC(=O)c3ccc(=O)n(-c4c(F)cccc4F)n3)cc(F)c21]

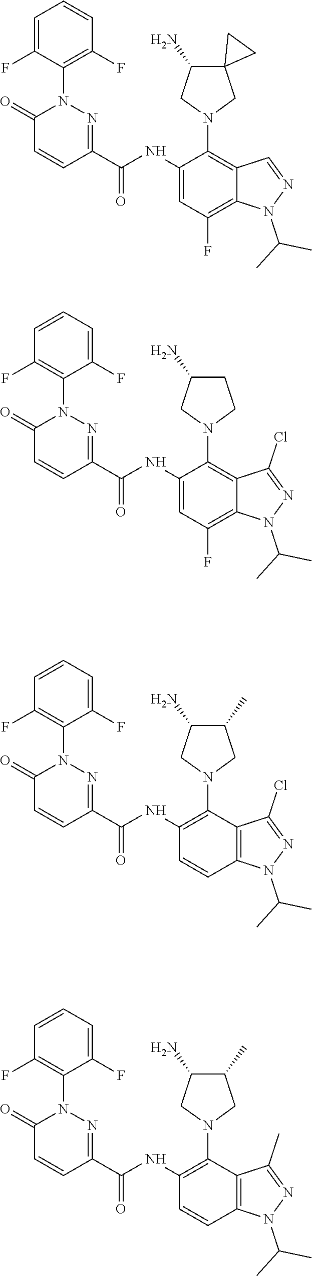 [SMILES: CC(C)n1ncc2c(N3C[C@H](N)C4(CC4)C3)c(NC(=O)c3ccc(=O)n(-c4c(F)cccc4F)n3)cc(F)c21 ; CC(C)n1nc(Cl)c2c(N3CC[C@@H](N)C3)c(NC(=O)c3ccc(=O)n(-c4c(F)cccc4F)n3)cc(F)c21 ; CC(C)n1nc(Cl)c2c(N3C[C@@H](C)[C@@H](N)C3)c(NC(=O)c3ccc(=O)n(-c4c(F)cccc4F)n3)ccc21 ; Cc1nn(C(C)C)c2ccc(NC(=O)c3ccc(=O)n(-c4c(F)cccc4F)n3)c(N3C[C@@H](C)[C@@H](N)C3)c12]

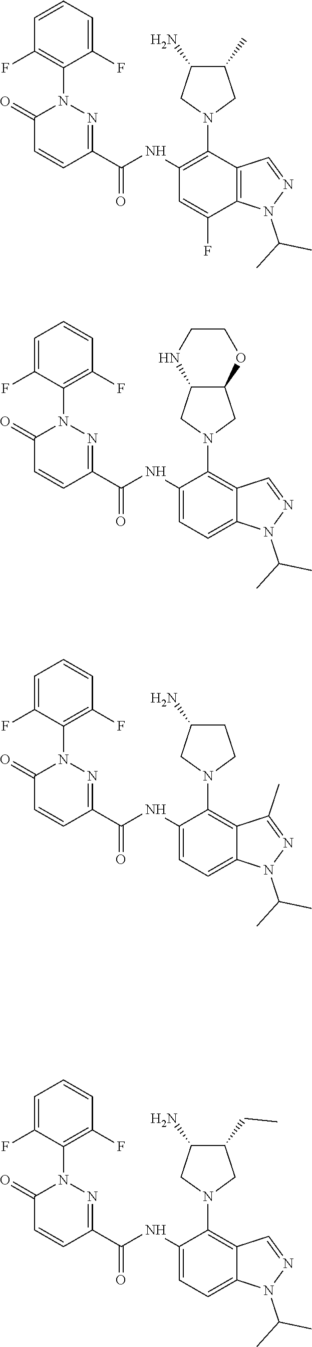 [SMILES: CC(C)n1ncc2c(N3C[C@@H](C)[C@@H](N)C3)c(NC(=O)c3ccc(=O)n(-c4c(F)cccc4F)n3)cc(F)c21 ; CC(C)n1ncc2c(N3C[C@@H]4NCCO[C@H]4C3)c(NC(=O)c3ccc(=O)n(-c4c(F)cccc4F)n3)ccc21 ; Cc1nn(C(C)C)c2ccc(NC(=O)c3ccc(=O)n(-c4c(F)cccc4F)n3)c(N3CC[C@@H](N)C3)c12 ; CC[C@@H]1CN(c2c(NC(=O)c3ccc(=O)n(-c4c(F)cccc4F)n3)ccc3c2cnn3C(C)C)C[C@@H]1N]

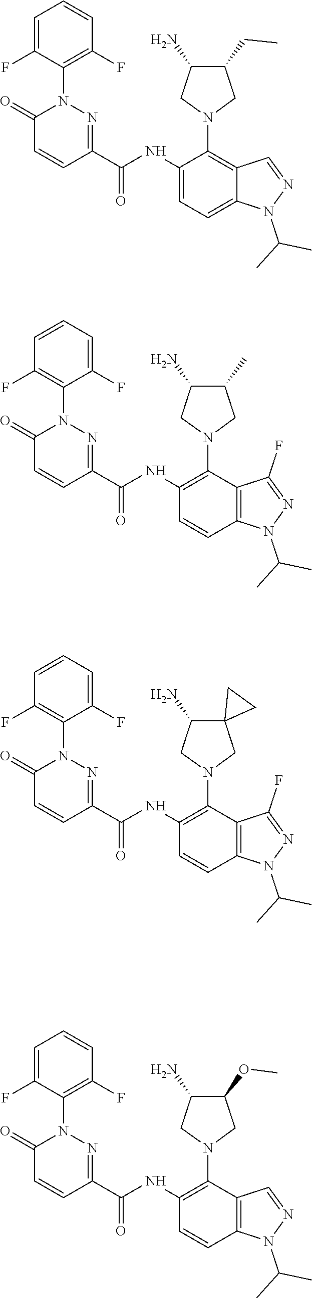 [SMILES: CC[C@@H]1CN(c2c(NC(=O)c3ccc(=O)n(-c4c(F)cccc4F)n3)ccc3c2cnn3C(C)C)C[C@@H]1N ; CC(C)n1nc(F)c2c(N3C[C@@H](C)[C@@H](N)C3)c(NC(=O)c3ccc(=O)n(-c4c(F)cccc4F)n3)ccc21 ; CC(C)n1nc(F)c2c(N3C[C@H](N)C4(CC4)C3)c(NC(=O)c3ccc(=O)n(-c4c(F)cccc4F)n3)ccc21 ; CO[C@H]1CN(c2c(NC(=O)c3ccc(=O)n(-c4c(F)cccc4F)n3)ccc3c2cnn3C(C)C)C[C@@H]1N]

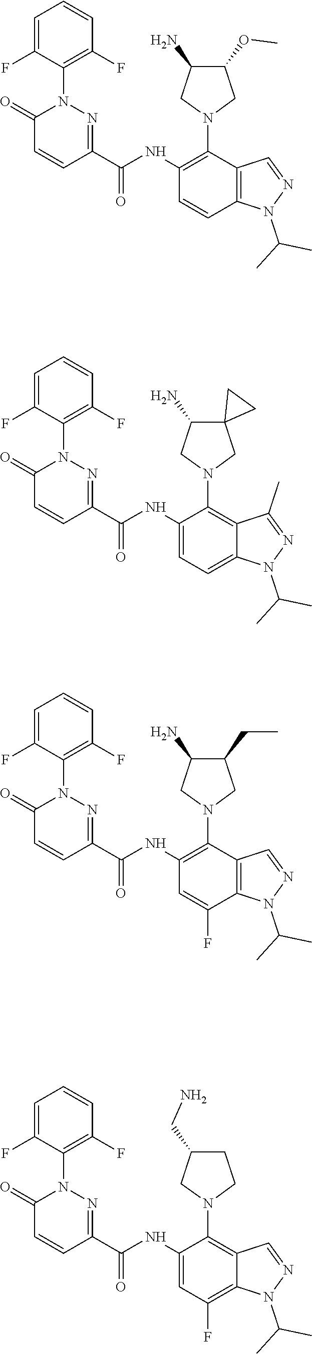 [SMILES: CO[C@@H]1CN(c2c(NC(=O)c3ccc(=O)n(-c4c(F)cccc4F)n3)ccc3c2cnn3C(C)C)C[C@H]1N ; Cc1nn(C(C)C)c2ccc(NC(=O)c3ccc(=O)n(-c4c(F)cccc4F)n3)c(N3C[C@H](N)C4(CC4)C3)c12 ; CC[C@H]1CN(c2c(NC(=O)c3ccc(=O)n(-c4c(F)cccc4F)n3)cc(F)c3c2cnn3C(C)C)C[C@H]1N ; CC(C)n1ncc2c(N3CC[C@@H](CN)C3)c(NC(=O)c3ccc(=O)n(-c4c(F)cccc4F)n3)cc(F)c21]

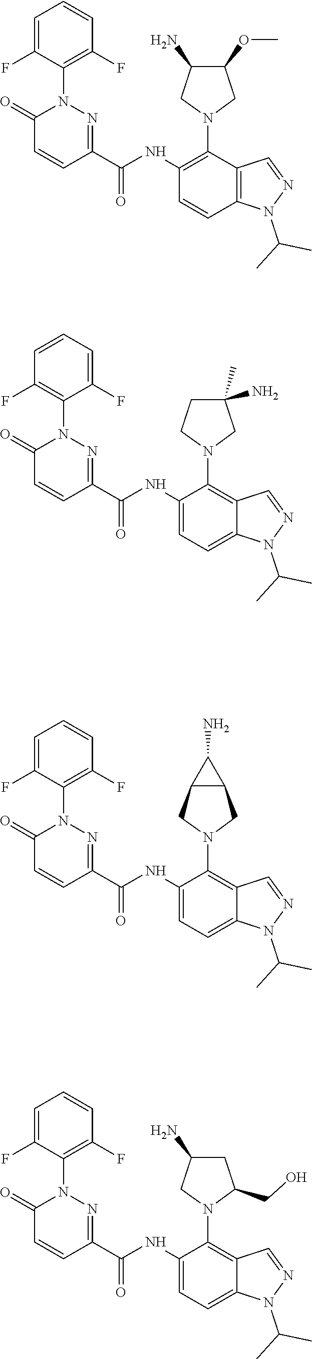 [SMILES: CO[C@H]1CN(c2c(NC(=O)c3ccc(=O)n(-c4c(F)cccc4F)n3)ccc3c2cnn3C(C)C)C[C@H]1N ; CC(C)n1ncc2c(N3CC[C@@](C)(N)C3)c(NC(=O)c3ccc(=O)n(-c4c(F)cccc4F)n3)ccc21 ; CC(C)n1ncc2c(N3C[C@@H]4[C@@H](N)[C@@H]4C3)c(NC(=O)c3ccc(=O)n(-c4c(F)cccc4F)n3)ccc21 ; CC(C)n1ncc2c(N3C[C@@H](N)C[C@H]3CO)c(NC(=O)c3ccc(=O)n(-c4c(F)cccc4F)n3)ccc21]

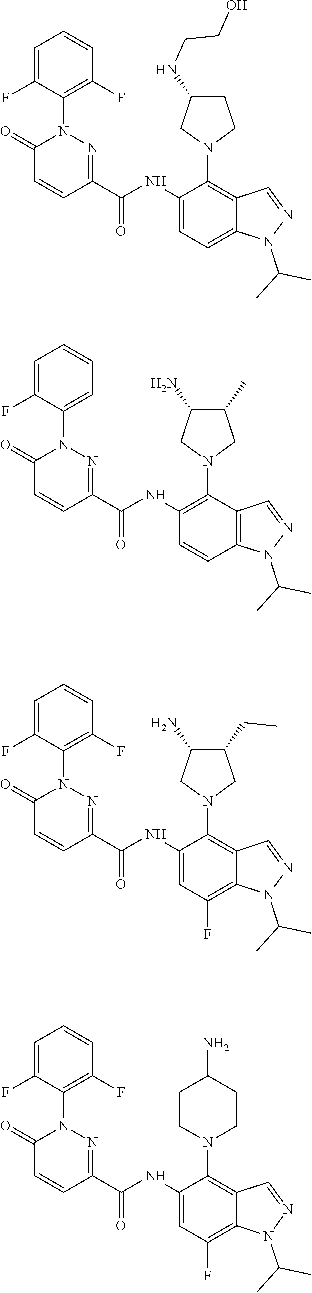 [SMILES: CC(C)n1ncc2c(N3CC[C@@H](NCCO)C3)c(NC(=O)c3ccc(=O)n(-c4c(F)cccc4F)n3)ccc21 ; CC(C)n1ncc2c(N3C[C@@H](C)[C@@H](N)C3)c(NC(=O)c3ccc(=O)n(-c4ccccc4F)n3)ccc21 ; CC[C@@H]1CN(c2c(NC(=O)c3ccc(=O)n(-c4c(F)cccc4F)n3)cc(F)c3c2cnn3C(C)C)C[C@@H]1N ; CC(C)n1ncc2c(N3CCC(N)CC3)c(NC(=O)c3ccc(=O)n(-c4c(F)cccc4F)n3)cc(F)c21]

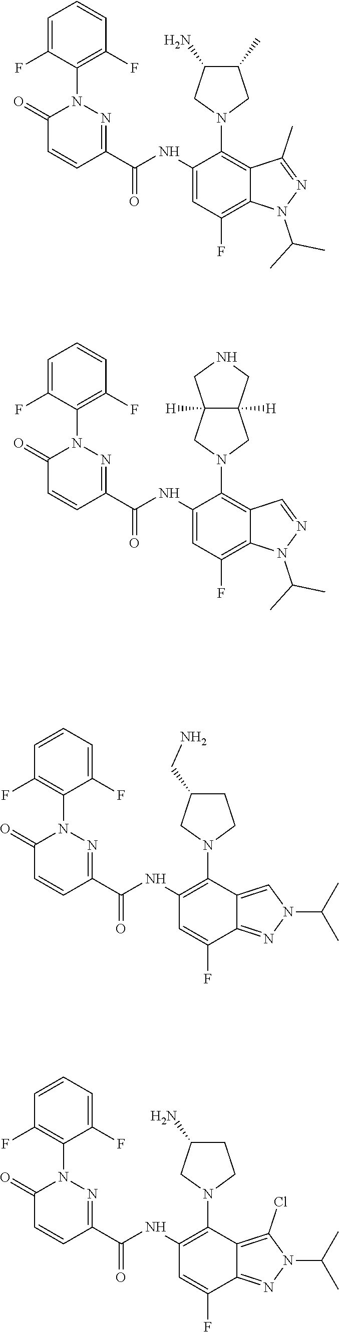 [SMILES: Cc1nn(C(C)C)c2c(F)cc(NC(=O)c3ccc(=O)n(-c4c(F)cccc4F)n3)c(N3C[C@@H](C)[C@@H](N)C3)c12 ; CC(C)n1ncc2c(N3C[C@H]4CNC[C@H]4C3)c(NC(=O)c3ccc(=O)n(-c4c(F)cccc4F)n3)cc(F)c21 ; CC(C)n1cc2c(N3CC[C@@H](CN)C3)c(NC(=O)c3ccc(=O)n(-c4c(F)cccc4F)n3)cc(F)c2n1 ; CC(C)n1nc2c(F)cc(NC(=O)c3ccc(=O)n(-c4c(F)cccc4F)n3)c(N3CC[C@@H](N)C3)c2c1Cl]

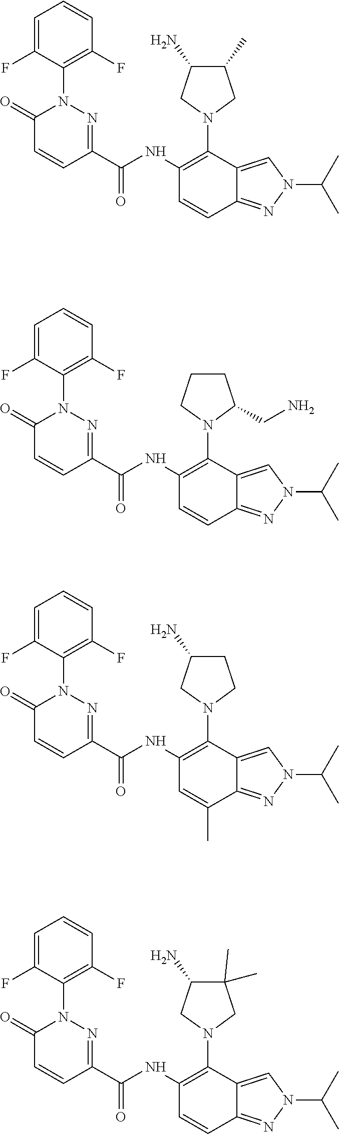 [SMILES: CC(C)n1cc2c(N3C[C@@H](C)[C@@H](N)C3)c(NC(=O)c3ccc(=O)n(-c4c(F)cccc4F)n3)ccc2n1 ; CC(C)n1cc2c(N3CCC[C@@H]3CN)c(NC(=O)c3ccc(=O)n(-c4c(F)cccc4F)n3)ccc2n1 ; Cc1cc(NC(=O)c2ccc(=O)n(-c3c(F)cccc3F)n2)c(N2CC[C@@H](N)C2)c2cn(C(C)C)nc12 ; CC(C)n1cc2c(N3C[C@H](N)C(C)(C)C3)c(NC(=O)c3ccc(=O)n(-c4c(F)cccc4F)n3)ccc2n1]

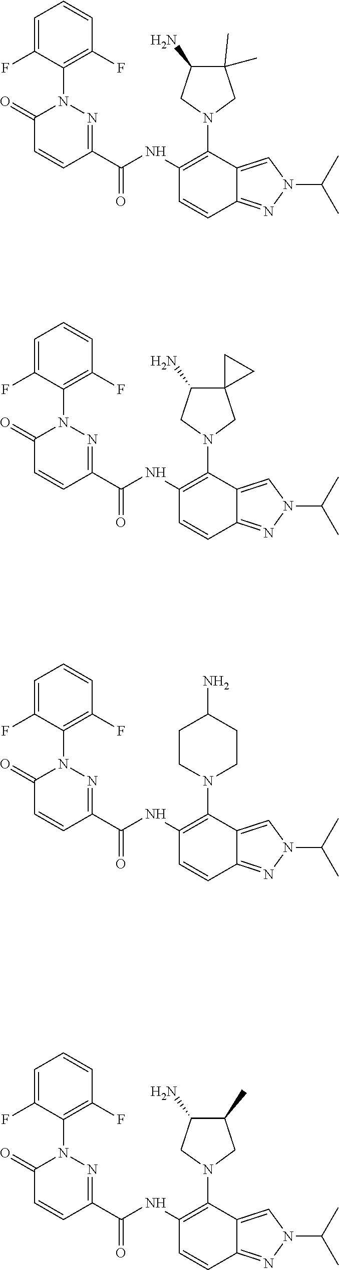 [SMILES: CC(C)n1cc2c(N3C[C@@H](N)C(C)(C)C3)c(NC(=O)c3ccc(=O)n(-c4c(F)cccc4F)n3)ccc2n1 ; CC(C)n1cc2c(N3C[C@H](N)C4(CC4)C3)c(NC(=O)c3ccc(=O)n(-c4c(F)cccc4F)n3)ccc2n1 ; CC(C)n1cc2c(N3CCC(N)CC3)c(NC(=O)c3ccc(=O)n(-c4c(F)cccc4F)n3)ccc2n1 ; CC(C)n1cc2c(N3C[C@H](C)[C@@H](N)C3)c(NC(=O)c3ccc(=O)n(-c4c(F)cccc4F)n3)ccc2n1]

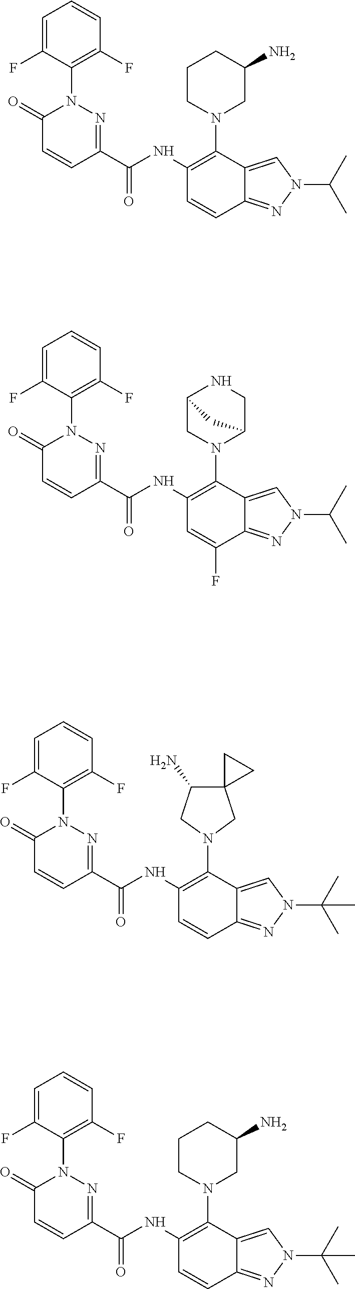 [SMILES: CC(C)n1cc2c(N3CCC[C@@H](N)C3)c(NC(=O)c3ccc(=O)n(-c4c(F)cccc4F)n3)ccc2n1 ; CC(C)n1cc2c(N3C[C@@H]4C[C@H]3CN4)c(NC(=O)c3ccc(=O)n(-c4c(F)cccc4F)n3)cc(F)c2n1 ; CC(C)(C)n1cc2c(N3C[C@H](N)C4(CC4)C3)c(NC(=O)c3ccc(=O)n(-c4c(F)cccc4F)n3)ccc2n1 ; CC(C)(C)n1cc2c(N3CCC[C@@H](N)C3)c(NC(=O)c3ccc(=O)n(-c4c(F)cccc4F)n3)ccc2n1]

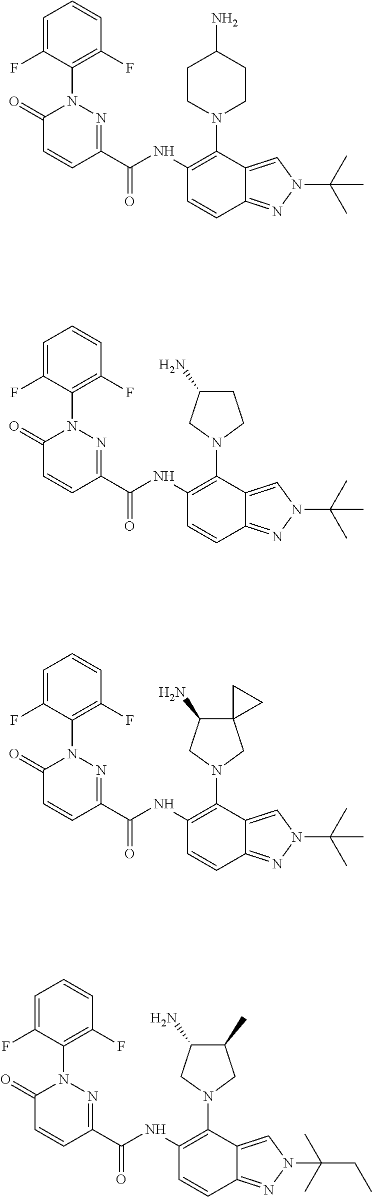 [SMILES: CC(C)(C)n1cc2c(N3CCC(N)CC3)c(NC(=O)c3ccc(=O)n(-c4c(F)cccc4F)n3)ccc2n1 ; CC(C)(C)n1cc2c(N3CC[C@@H](N)C3)c(NC(=O)c3ccc(=O)n(-c4c(F)cccc4F)n3)ccc2n1 ; CC(C)(C)n1cc2c(N3C[C@@H](N)C4(CC4)C3)c(NC(=O)c3ccc(=O)n(-c4c(F)cccc4F)n3)ccc2n1 ; CCC(C)(C)n1cc2c(N3C[C@H](C)[C@@H](N)C3)c(NC(=O)c3ccc(=O)n(-c4c(F)cccc4F)n3)ccc2n1]

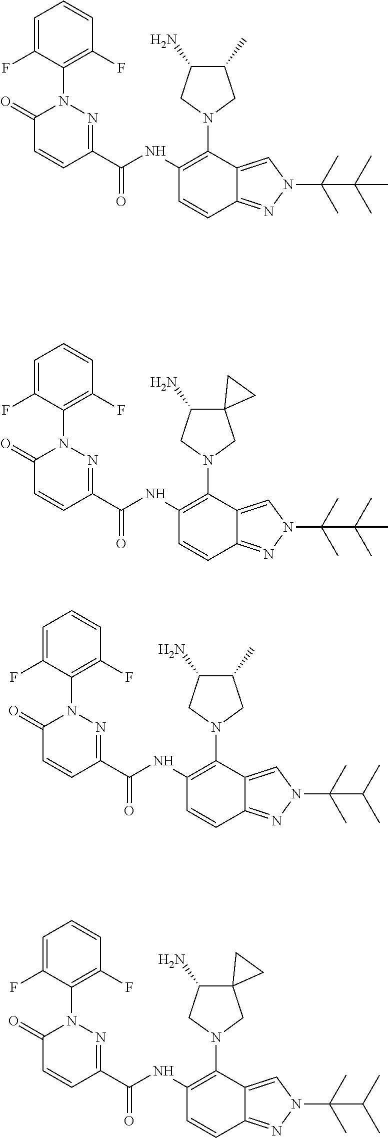 [SMILES: C[C@@H]1CN(c2c(NC(=O)c3ccc(=O)n(-c4c(F)cccc4F)n3)ccc3nn(C(C)(C)C(C)(C)C)cc23)C[C@@H]1N ; CC(C)(C)C(C)(C)n1cc2c(N3C[C@H](N)C4(CC4)C3)c(NC(=O)c3ccc(=O)n(-c4c(F)cccc4F)n3)ccc2n1 ; CC(C)C(C)(C)n1cc2c(N3C[C@@H](C)[C@@H](N)C3)c(NC(=O)c3ccc(=O)n(-c4c(F)cccc4F)n3)ccc2n1 ; CC(C)C(C)(C)n1cc2c(N3C[C@H](N)C4(CC4)C3)c(NC(=O)c3ccc(=O)n(-c4c(F)cccc4F)n3)ccc2n1]

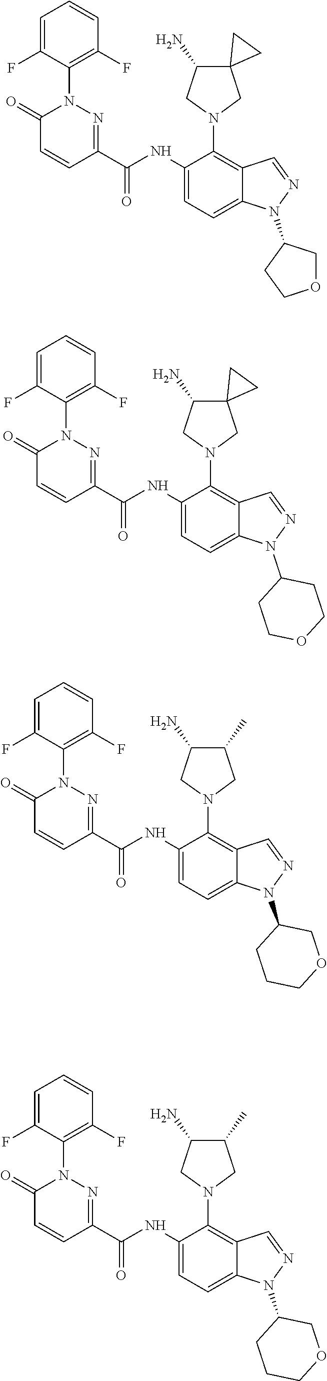 [SMILES: N[C@H]1CN(c2c(NC(=O)c3ccc(=O)n(-c4c(F)cccc4F)n3)ccc3c2cnn3[C@H]2CCOC2)CC12CC2 ; N[C@H]1CN(c2c(NC(=O)c3ccc(=O)n(-c4c(F)cccc4F)n3)ccc3c2cnn3C2CCOCC2)CC12CC2 ; C[C@@H]1CN(c2c(NC(=O)c3ccc(=O)n(-c4c(F)cccc4F)n3)ccc3c2cnn3[C@@H]2CCCOC2)C[C@@H]1N ; C[C@@H]1CN(c2c(NC(=O)c3ccc(=O)n(-c4c(F)cccc4F)n3)ccc3c2cnn3[C@H]2CCCOC2)C[C@@H]1N]

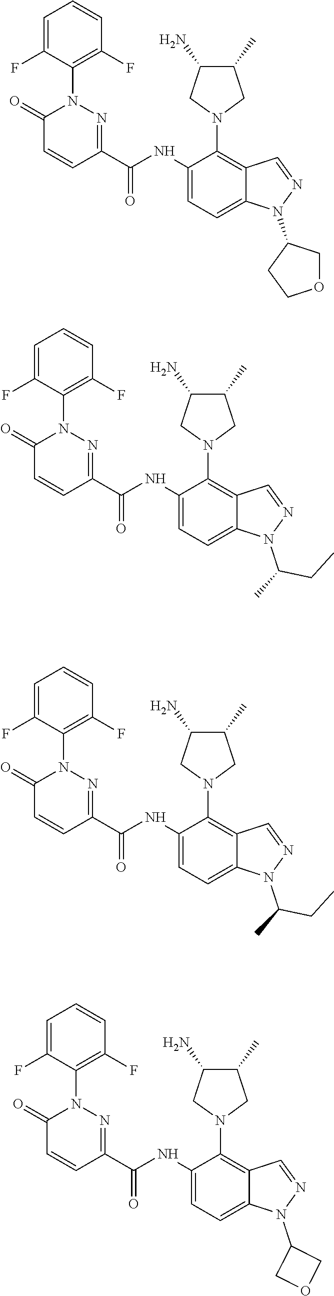 [SMILES: C[C@@H]1CN(c2c(NC(=O)c3ccc(=O)n(-c4c(F)cccc4F)n3)ccc3c2cnn3[C@H]2CCOC2)C[C@@H]1N ; CC[C@H](C)n1ncc2c(N3C[C@@H](C)[C@@H](N)C3)c(NC(=O)c3ccc(=O)n(-c4c(F)cccc4F)n3)ccc21 ; CC[C@@H](C)n1ncc2c(N3C[C@@H](C)[C@@H](N)C3)c(NC(=O)c3ccc(=O)n(-c4c(F)cccc4F)n3)ccc21 ; C[C@@H]1CN(c2c(NC(=O)c3ccc(=O)n(-c4c(F)cccc4F)n3)ccc3c2cnn3C2COC2)C[C@@H]1N]

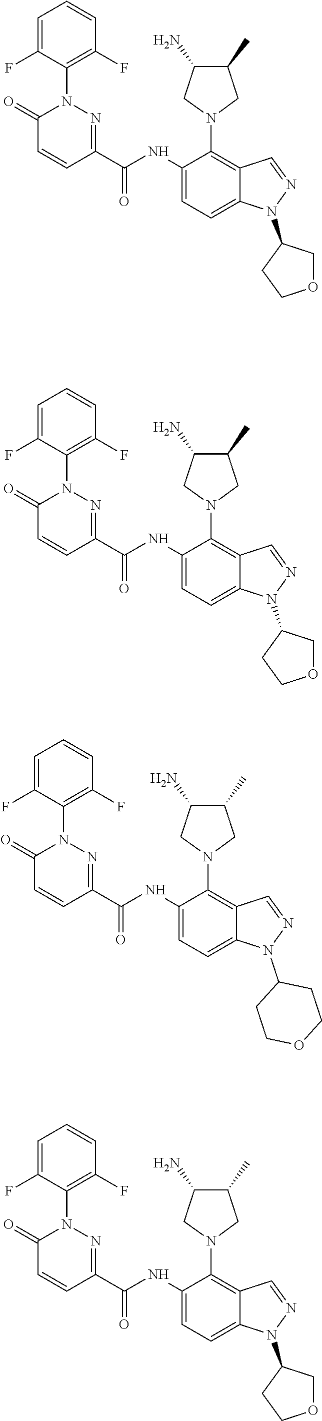 [SMILES: C[C@H]1CN(c2c(NC(=O)c3ccc(=O)n(-c4c(F)cccc4F)n3)ccc3c2cnn3[C@@H]2CCOC2)C[C@@H]1N ; C[C@H]1CN(c2c(NC(=O)c3ccc(=O)n(-c4c(F)cccc4F)n3)ccc3c2cnn3[C@H]2CCOC2)C[C@@H]1N ; C[C@@H]1CN(c2c(NC(=O)c3ccc(=O)n(-c4c(F)cccc4F)n3)ccc3c2cnn3C2CCOCC2)C[C@@H]1N ; C[C@@H]1CN(c2c(NC(=O)c3ccc(=O)n(-c4c(F)cccc4F)n3)ccc3c2cnn3[C@@H]2CCOC2)C[C@@H]1N]

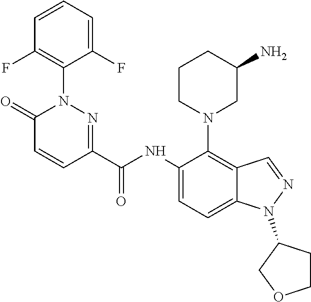 [SMILES: N[C@@H]1CCCN(c2c(NC(=O)c3ccc(=O)n(-c4c(F)cccc4F)n3)ccc3c2cnn3[C@@H]2CCOC2)C1]

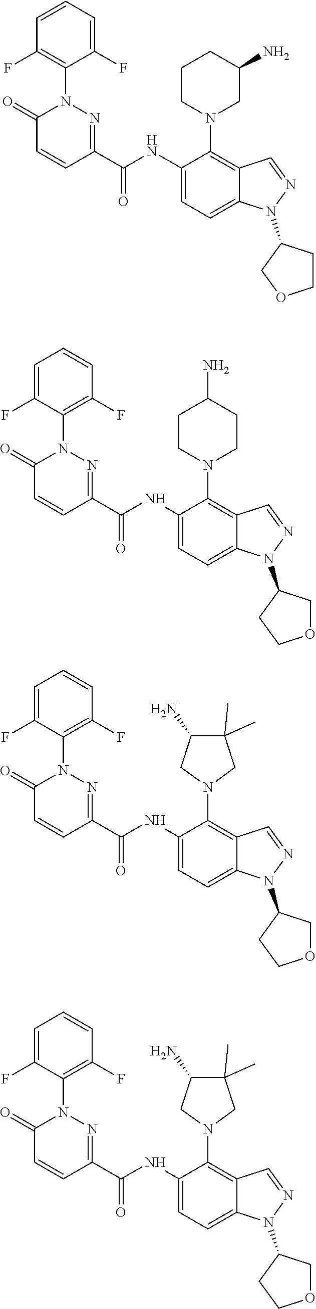 [SMILES: N[C@@H]1CCCN(c2c(NC(=O)c3ccc(=O)n(-c4c(F)cccc4F)n3)ccc3c2cnn3[C@@H]2CCOC2)C1 ; NC1CCN(c2c(NC(=O)c3ccc(=O)n(-c4c(F)cccc4F)n3)ccc3c2cnn3[C@@H]2CCOC2)CC1 ; CC1(C)CN(c2c(NC(=O)c3ccc(=O)n(-c4c(F)cccc4F)n3)ccc3c2cnn3[C@@H]2CCOC2)C[C@@H]1N ; CC1(C)CN(c2c(NC(=O)c3ccc(=O)n(-c4c(F)cccc4F)n3)ccc3c2cnn3[C@H]2CCOC2)C[C@@H]1N]

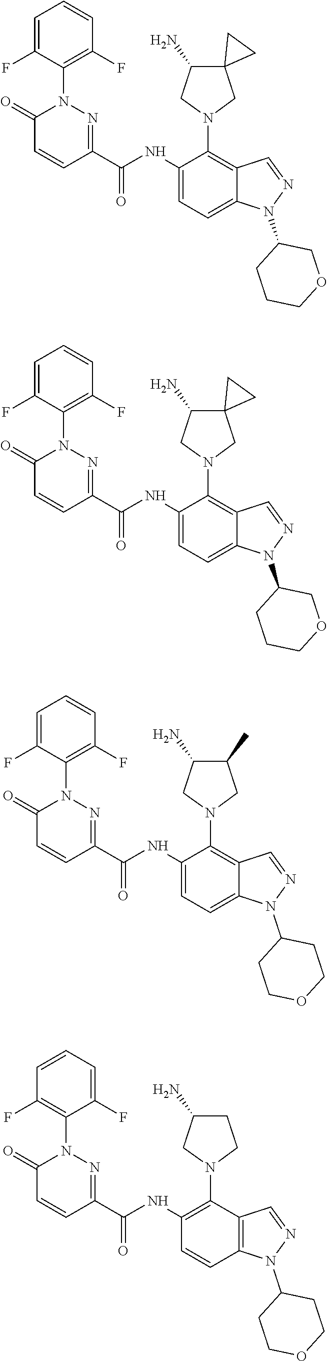 [SMILES: N[C@H]1CN(c2c(NC(=O)c3ccc(=O)n(-c4c(F)cccc4F)n3)ccc3c2cnn3[C@H]2CCCOC2)CC12CC2 ; N[C@H]1CN(c2c(NC(=O)c3ccc(=O)n(-c4c(F)cccc4F)n3)ccc3c2cnn3[C@@H]2CCCOC2)CC12CC2 ; C[C@H]1CN(c2c(NC(=O)c3ccc(=O)n(-c4c(F)cccc4F)n3)ccc3c2cnn3C2CCOCC2)C[C@@H]1N ; N[C@@H]1CCN(c2c(NC(=O)c3ccc(=O)n(-c4c(F)cccc4F)n3)ccc3c2cnn3C2CCOCC2)C1]

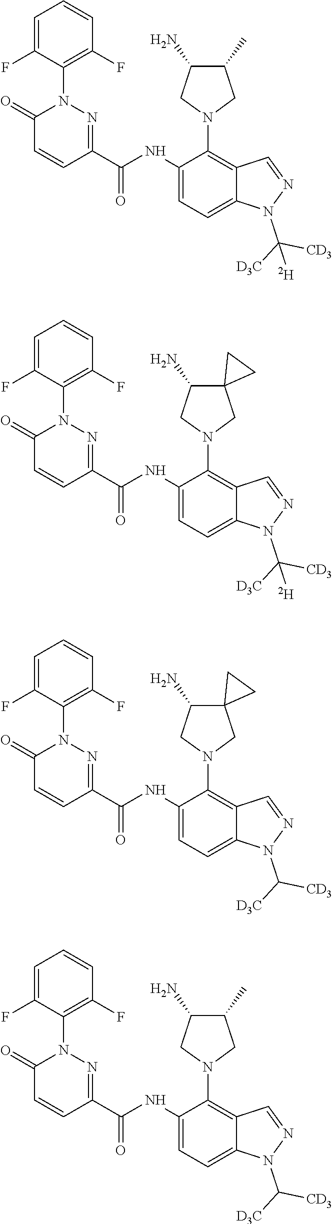 [SMILES: [2H]C([2H])([2H])C([2H])(n1ncc2c(N3C[C@@H](C)[C@@H](N)C3)c(NC(=O)c3ccc(=O)n(-c4c(F)cccc4F)n3)ccc21)C([2H])([2H])[2H] ; [2H]C([2H])([2H])C([2H])(n1ncc2c(N3C[C@H](N)C4(CC4)C3)c(NC(=O)c3ccc(=O)n(-c4c(F)cccc4F)n3)ccc21)C([2H])([2H])[2H] ; [2H]C([2H])([2H])C(n1ncc2c(N3C[C@H](N)C4(CC4)C3)c(NC(=O)c3ccc(=O)n(-c4c(F)cccc4F)n3)ccc21)C([2H])([2H])[2H] ; [2H]C([2H])([2H])C(n1ncc2c(N3C[C@@H](C)[C@@H](N)C3)c(NC(=O)c3ccc(=O)n(-c4c(F)cccc4F)n3)ccc21)C([2H])([2H])[2H]]

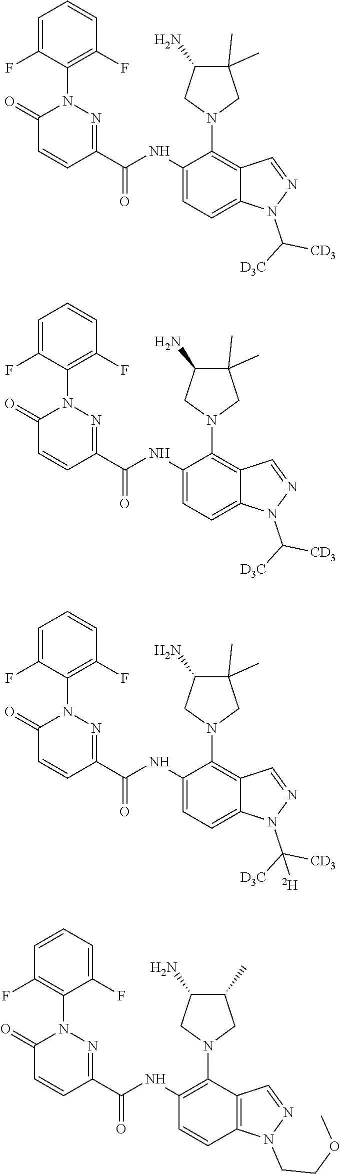 [SMILES: [2H]C([2H])([2H])C(n1ncc2c(N3C[C@H](N)C(C)(C)C3)c(NC(=O)c3ccc(=O)n(-c4c(F)cccc4F)n3)ccc21)C([2H])([2H])[2H] ; [2H]C([2H])([2H])C(n1ncc2c(N3C[C@@H](N)C(C)(C)C3)c(NC(=O)c3ccc(=O)n(-c4c(F)cccc4F)n3)ccc21)C([2H])([2H])[2H] ; [2H]C([2H])([2H])C([2H])(n1ncc2c(N3C[C@H](N)C(C)(C)C3)c(NC(=O)c3ccc(=O)n(-c4c(F)cccc4F)n3)ccc21)C([2H])([2H])[2H] ; COCCn1ncc2c(N3C[C@@H](C)[C@@H](N)C3)c(NC(=O)c3ccc(=O)n(-c4c(F)cccc4F)n3)ccc21]

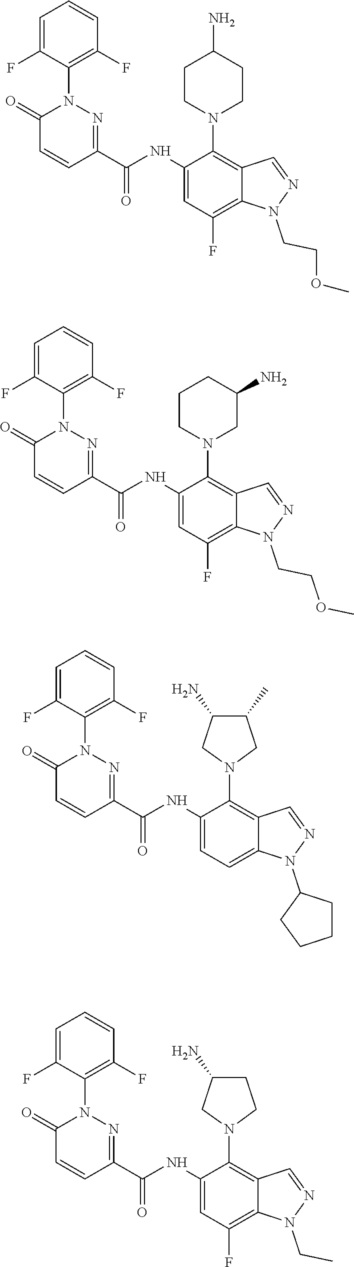 [SMILES: COCCn1ncc2c(N3CCC(N)CC3)c(NC(=O)c3ccc(=O)n(-c4c(F)cccc4F)n3)cc(F)c21 ; COCCn1ncc2c(N3CCC[C@@H](N)C3)c(NC(=O)c3ccc(=O)n(-c4c(F)cccc4F)n3)cc(F)c21 ; C[C@@H]1CN(c2c(NC(=O)c3ccc(=O)n(-c4c(F)cccc4F)n3)ccc3c2cnn3C2CCCC2)C[C@@H]1N ; CCn1ncc2c(N3CC[C@@H](N)C3)c(NC(=O)c3ccc(=O)n(-c4c(F)cccc4F)n3)cc(F)c21]

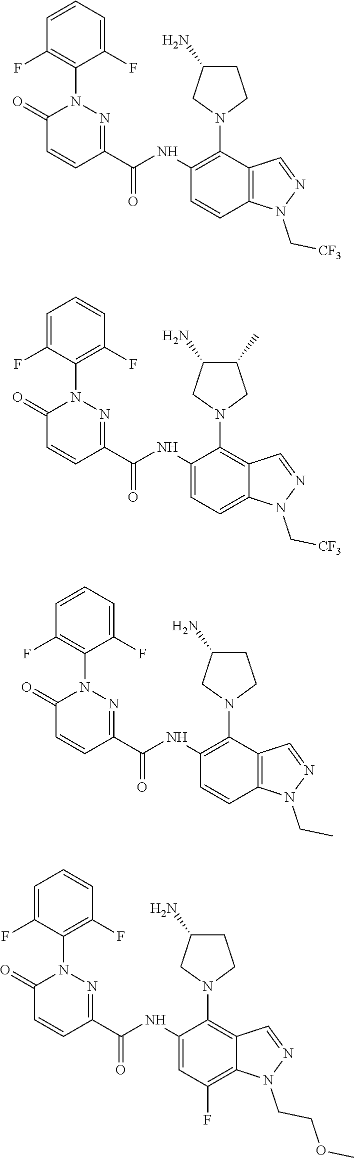 [SMILES: N[C@@H]1CCN(c2c(NC(=O)c3ccc(=O)n(-c4c(F)cccc4F)n3)ccc3c2cnn3CC(F)(F)F)C1 ; C[C@@H]1CN(c2c(NC(=O)c3ccc(=O)n(-c4c(F)cccc4F)n3)ccc3c2cnn3CC(F)(F)F)C[C@@H]1N ; CCn1ncc2c(N3CC[C@@H](N)C3)c(NC(=O)c3ccc(=O)n(-c4c(F)cccc4F)n3)ccc21 ; COCCn1ncc2c(N3CC[C@@H](N)C3)c(NC(=O)c3ccc(=O)n(-c4c(F)cccc4F)n3)cc(F)c21]

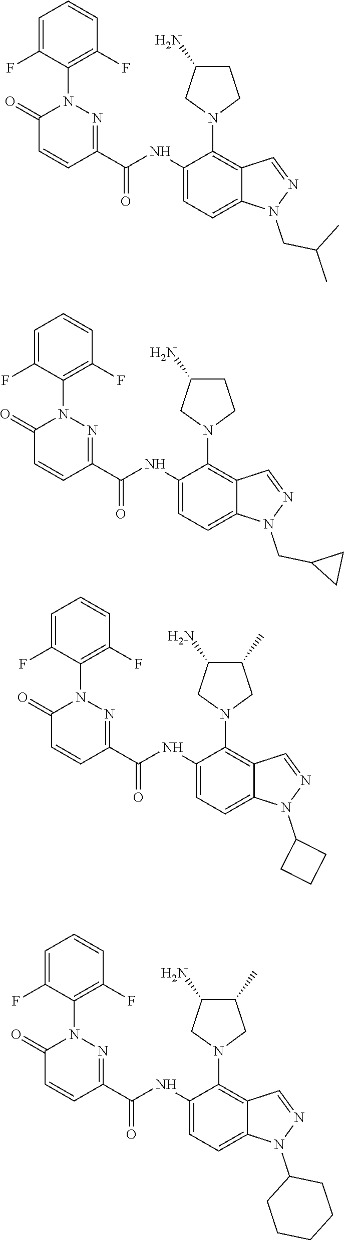 [SMILES: CC(C)Cn1ncc2c(N3CC[C@@H](N)C3)c(NC(=O)c3ccc(=O)n(-c4c(F)cccc4F)n3)ccc21 ; N[C@@H]1CCN(c2c(NC(=O)c3ccc(=O)n(-c4c(F)cccc4F)n3)ccc3c2cnn3CC2CC2)C1 ; C[C@@H]1CN(c2c(NC(=O)c3ccc(=O)n(-c4c(F)cccc4F)n3)ccc3c2cnn3C2CCC2)C[C@@H]1N ; C[C@@H]1CN(c2c(NC(=O)c3ccc(=O)n(-c4c(F)cccc4F)n3)ccc3c2cnn3C2CCCCC2)C[C@@H]1N]

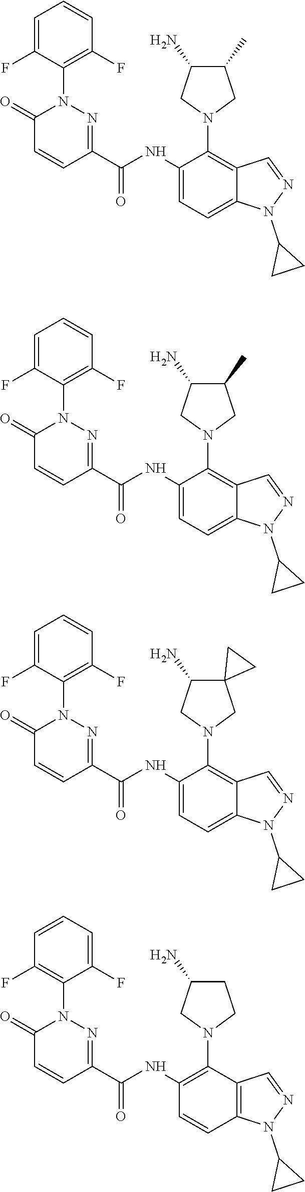 [SMILES: C[C@@H]1CN(c2c(NC(=O)c3ccc(=O)n(-c4c(F)cccc4F)n3)ccc3c2cnn3C2CC2)C[C@@H]1N ; C[C@H]1CN(c2c(NC(=O)c3ccc(=O)n(-c4c(F)cccc4F)n3)ccc3c2cnn3C2CC2)C[C@@H]1N ; N[C@H]1CN(c2c(NC(=O)c3ccc(=O)n(-c4c(F)cccc4F)n3)ccc3c2cnn3C2CC2)CC12CC2 ; N[C@@H]1CCN(c2c(NC(=O)c3ccc(=O)n(-c4c(F)cccc4F)n3)ccc3c2cnn3C2CC2)C1]

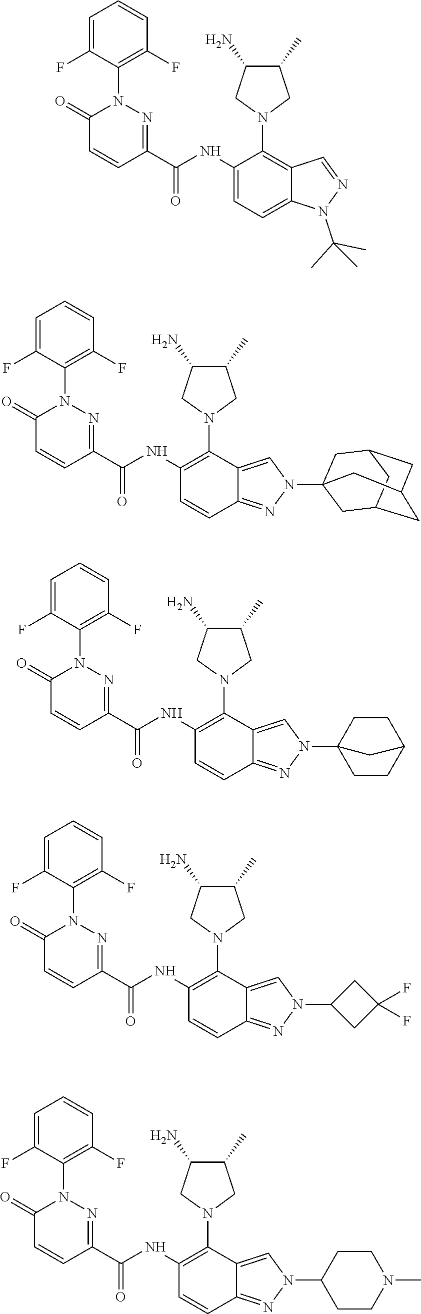 [SMILES: C[C@@H]1CN(c2c(NC(=O)c3ccc(=O)n(-c4c(F)cccc4F)n3)ccc3c2cnn3C(C)(C)C)C[C@@H]1N ; C[C@@H]1CN(c2c(NC(=O)c3ccc(=O)n(-c4c(F)cccc4F)n3)ccc3nn(C45CC6CC(CC(C6)C4)C5)cc23)C[C@@H]1N ; C[C@@H]1CN(c2c(NC(=O)c3ccc(=O)n(-c4c(F)cccc4F)n3)ccc3nn(C45CCC(CC4)C5)cc23)C[C@@H]1N ; C[C@@H]1CN(c2c(NC(=O)c3ccc(=O)n(-c4c(F)cccc4F)n3)ccc3nn(C4CC(F)(F)C4)cc23)C[C@@H]1N ; C[C@@H]1CN(c2c(NC(=O)c3ccc(=O)n(-c4c(F)cccc4F)n3)ccc3nn(C4CCN(C)CC4)cc23)C[C@@H]1N]

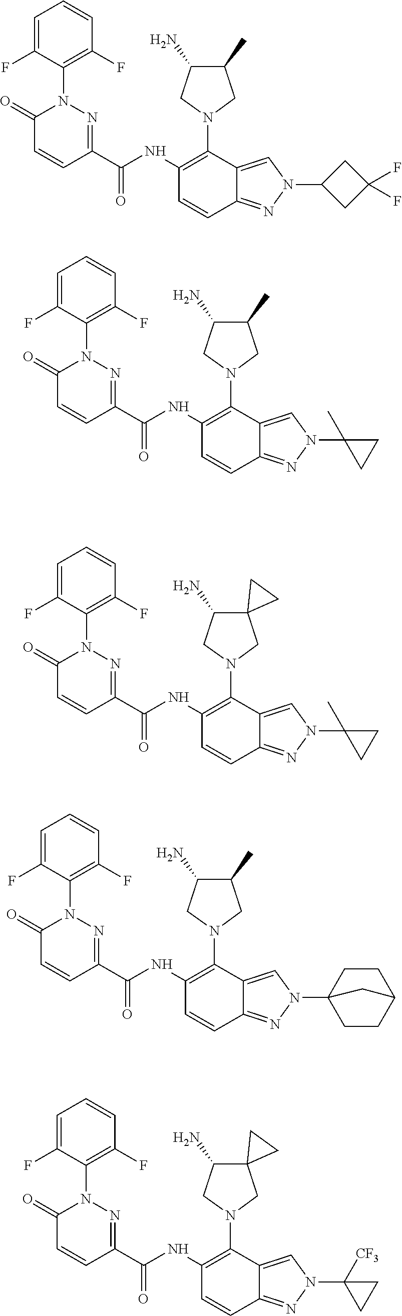 [SMILES: C[C@H]1CN(c2c(NC(=O)c3ccc(=O)n(-c4c(F)cccc4F)n3)ccc3nn(C4CC(F)(F)C4)cc23)C[C@@H]1N ; C[C@H]1CN(c2c(NC(=O)c3ccc(=O)n(-c4c(F)cccc4F)n3)ccc3nn(C4(C)CC4)cc23)C[C@@H]1N ; CC1(n2cc3c(N4C[C@H](N)C5(CC5)C4)c(NC(=O)c4ccc(=O)n(-c5c(F)cccc5F)n4)ccc3n2)CC1 ; C[C@H]1CN(c2c(NC(=O)c3ccc(=O)n(-c4c(F)cccc4F)n3)ccc3nn(C45CCC(CC4)C5)cc23)C[C@@H]1N ; N[C@H]1CN(c2c(NC(=O)c3ccc(=O)n(-c4c(F)cccc4F)n3)ccc3nn(C4(C(F)(F)F)CC4)cc23)CC12CC2]

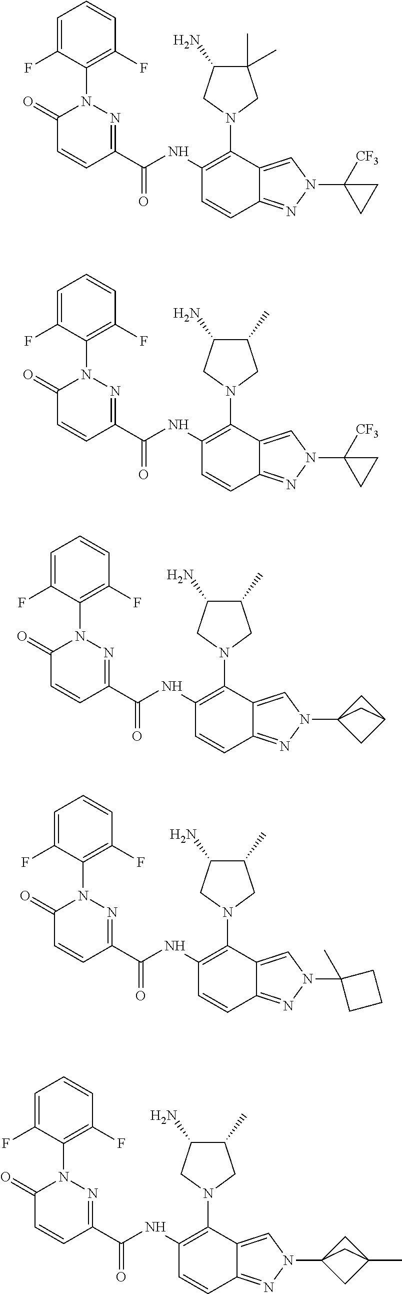 [SMILES: CC1(C)CN(c2c(NC(=O)c3ccc(=O)n(-c4c(F)cccc4F)n3)ccc3nn(C4(C(F)(F)F)CC4)cc23)C[C@@H]1N ; C[C@@H]1CN(c2c(NC(=O)c3ccc(=O)n(-c4c(F)cccc4F)n3)ccc3nn(C4(C(F)(F)F)CC4)cc23)C[C@@H]1N ; C[C@@H]1CN(c2c(NC(=O)c3ccc(=O)n(-c4c(F)cccc4F)n3)ccc3nn(C45CC(C4)C5)cc23)C[C@@H]1N ; C[C@@H]1CN(c2c(NC(=O)c3ccc(=O)n(-c4c(F)cccc4F)n3)ccc3nn(C4(C)CCC4)cc23)C[C@@H]1N ; C[C@@H]1CN(c2c(NC(=O)c3ccc(=O)n(-c4c(F)cccc4F)n3)ccc3nn(C45CC(C)(C4)C5)cc23)C[C@@H]1N]

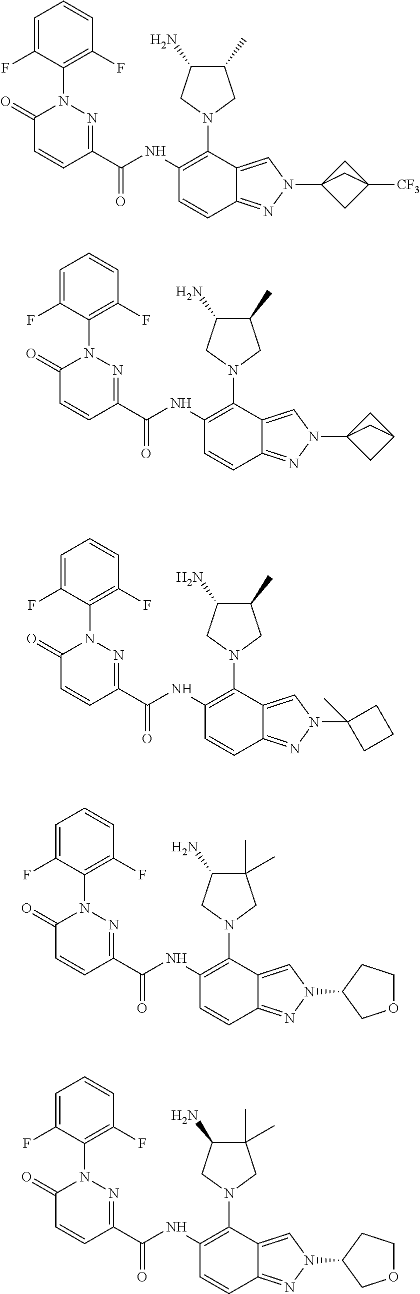 [SMILES: C[C@@H]1CN(c2c(NC(=O)c3ccc(=O)n(-c4c(F)cccc4F)n3)ccc3nn(C45CC(C(F)(F)F)(C4)C5)cc23)C[C@@H]1N ; C[C@H]1CN(c2c(NC(=O)c3ccc(=O)n(-c4c(F)cccc4F)n3)ccc3nn(C45CC(C4)C5)cc23)C[C@@H]1N ; C[C@H]1CN(c2c(NC(=O)c3ccc(=O)n(-c4c(F)cccc4F)n3)ccc3nn(C4(C)CCC4)cc23)C[C@@H]1N ; CC1(C)CN(c2c(NC(=O)c3ccc(=O)n(-c4c(F)cccc4F)n3)ccc3nn([C@@H]4CCOC4)cc23)C[C@@H]1N ; CC1(C)CN(c2c(NC(=O)c3ccc(=O)n(-c4c(F)cccc4F)n3)ccc3nn([C@@H]4CCOC4)cc23)C[C@H]1N]

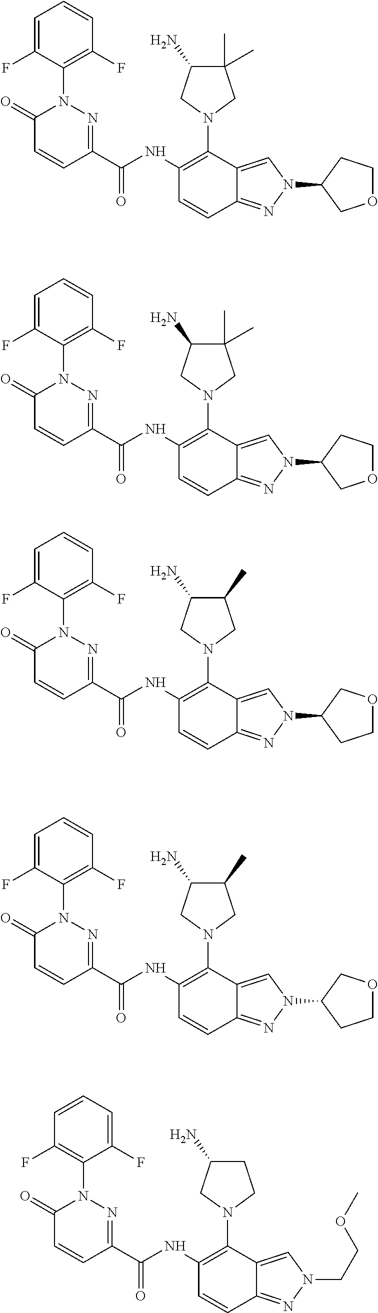 [SMILES: CC1(C)CN(c2c(NC(=O)c3ccc(=O)n(-c4c(F)cccc4F)n3)ccc3nn([C@H]4CCOC4)cc23)C[C@@H]1N ; CC1(C)CN(c2c(NC(=O)c3ccc(=O)n(-c4c(F)cccc4F)n3)ccc3nn([C@H]4CCOC4)cc23)C[C@H]1N ; C[C@H]1CN(c2c(NC(=O)c3ccc(=O)n(-c4c(F)cccc4F)n3)ccc3nn([C@@H]4CCOC4)cc23)C[C@@H]1N ; C[C@H]1CN(c2c(NC(=O)c3ccc(=O)n(-c4c(F)cccc4F)n3)ccc3nn([C@H]4CCOC4)cc23)C[C@@H]1N ; COCCn1cc2c(N3CC[C@@H](N)C3)c(NC(=O)c3ccc(=O)n(-c4c(F)cccc4F)n3)ccc2n1]

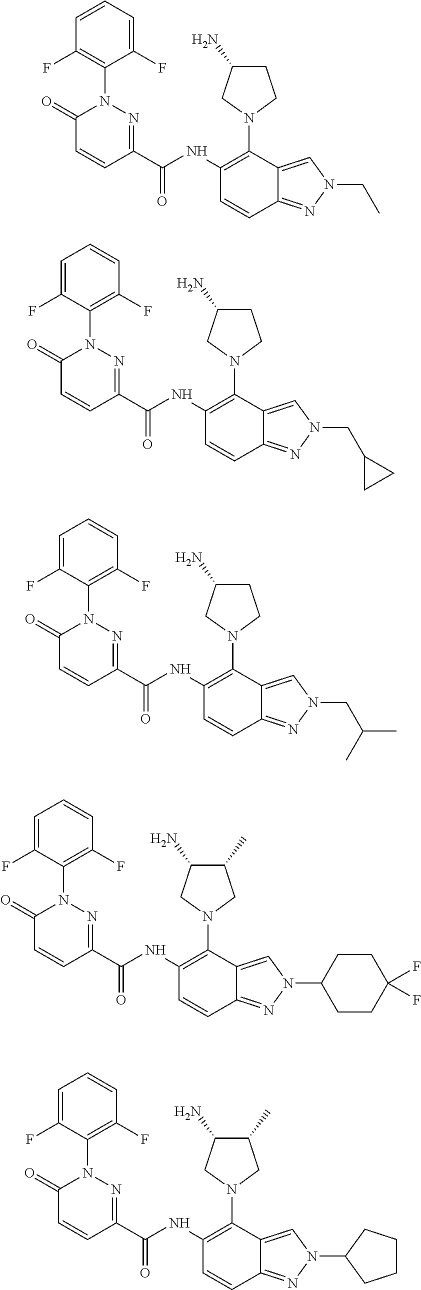 [SMILES: CCn1cc2c(N3CC[C@@H](N)C3)c(NC(=O)c3ccc(=O)n(-c4c(F)cccc4F)n3)ccc2n1 ; N[C@@H]1CCN(c2c(NC(=O)c3ccc(=O)n(-c4c(F)cccc4F)n3)ccc3nn(CC4CC4)cc23)C1 ; CC(C)Cn1cc2c(N3CC[C@@H](N)C3)c(NC(=O)c3ccc(=O)n(-c4c(F)cccc4F)n3)ccc2n1 ; C[C@@H]1CN(c2c(NC(=O)c3ccc(=O)n(-c4c(F)cccc4F)n3)ccc3nn(C4CCC(F)(F)CC4)cc23)C[C@@H]1N ; C[C@@H]1CN(c2c(NC(=O)c3ccc(=O)n(-c4c(F)cccc4F)n3)ccc3nn(C4CCCC4)cc23)C[C@@H]1N]

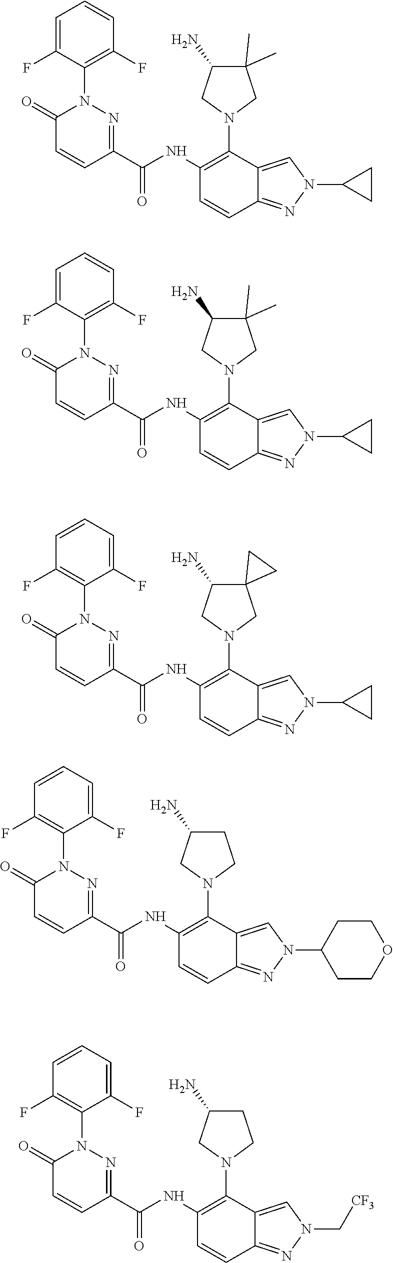 [SMILES: CC1(C)CN(c2c(NC(=O)c3ccc(=O)n(-c4c(F)cccc4F)n3)ccc3nn(C4CC4)cc23)C[C@@H]1N ; CC1(C)CN(c2c(NC(=O)c3ccc(=O)n(-c4c(F)cccc4F)n3)ccc3nn(C4CC4)cc23)C[C@H]1N ; N[C@H]1CN(c2c(NC(=O)c3ccc(=O)n(-c4c(F)cccc4F)n3)ccc3nn(C4CC4)cc23)CC12CC2 ; N[C@@H]1CCN(c2c(NC(=O)c3ccc(=O)n(-c4c(F)cccc4F)n3)ccc3nn(C4CCOCC4)cc23)C1 ; N[C@@H]1CCN(c2c(NC(=O)c3ccc(=O)n(-c4c(F)cccc4F)n3)ccc3nn(CC(F)(F)F)cc23)C1]

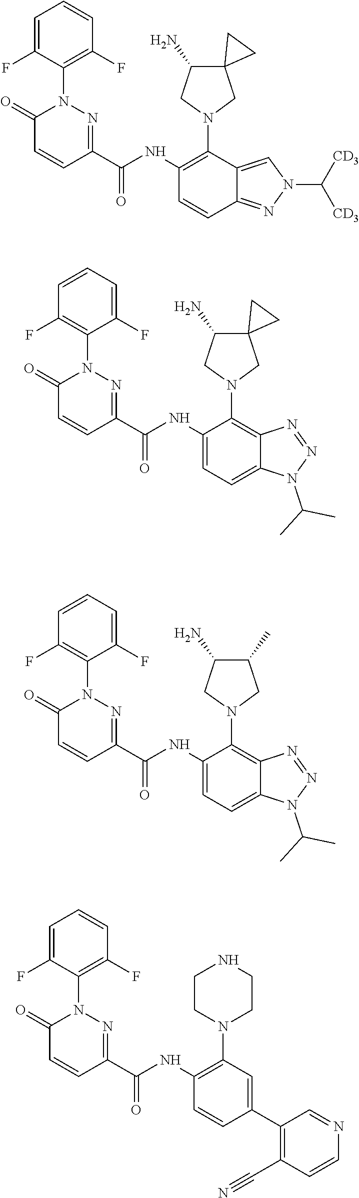 [SMILES: [2H]C([2H])([2H])C(n1cc2c(N3C[C@H](N)C4(CC4)C3)c(NC(=O)c3ccc(=O)n(-c4c(F)cccc4F)n3)ccc2n1)C([2H])([2H])[2H] ; CC(C)n1nnc2c(N3C[C@H](N)C4(CC4)C3)c(NC(=O)c3ccc(=O)n(-c4c(F)cccc4F)n3)ccc21 ; CC(C)n1nnc2c(N3C[C@@H](C)[C@@H](N)C3)c(NC(=O)c3ccc(=O)n(-c4c(F)cccc4F)n3)ccc21 ; N#Cc1ccncc1-c1ccc(NC(=O)c2ccc(=O)n(-c3c(F)cccc3F)n2)c(N2CCNCC2)c1]

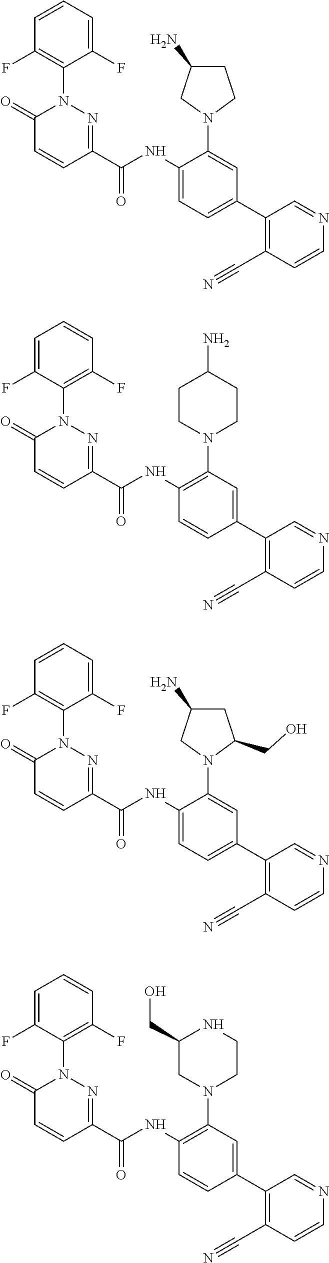 [SMILES: N#Cc1ccncc1-c1ccc(NC(=O)c2ccc(=O)n(-c3c(F)cccc3F)n2)c(N2CC[C@H](N)C2)c1 ; N#Cc1ccncc1-c1ccc(NC(=O)c2ccc(=O)n(-c3c(F)cccc3F)n2)c(N2CCC(N)CC2)c1 ; N#Cc1ccncc1-c1ccc(NC(=O)c2ccc(=O)n(-c3c(F)cccc3F)n2)c(N2C[C@@H](N)C[C@H]2CO)c1 ; N#Cc1ccncc1-c1ccc(NC(=O)c2ccc(=O)n(-c3c(F)cccc3F)n2)c(N2CCN[C@H](CO)C2)c1]

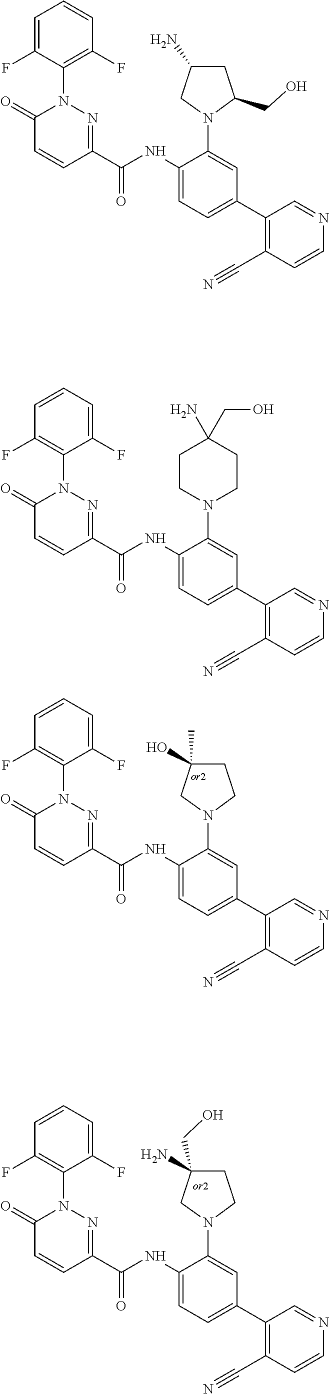 [SMILES: N#Cc1ccncc1-c1ccc(NC(=O)c2ccc(=O)n(-c3c(F)cccc3F)n2)c(N2C[C@H](N)C[C@H]2CO)c1 ; N#Cc1ccncc1-c1ccc(NC(=O)c2ccc(=O)n(-c3c(F)cccc3F)n2)c(N2CCC(N)(CO)CC2)c1 ; C[C@]1(O)CCN(c2cc(-c3cnccc3C#N)ccc2NC(=O)c2ccc(=O)n(-c3c(F)cccc3F)n2)C1 ; N#Cc1ccncc1-c1ccc(NC(=O)c2ccc(=O)n(-c3c(F)cccc3F)n2)c(N2CC[C@@](N)(CO)C2)c1]

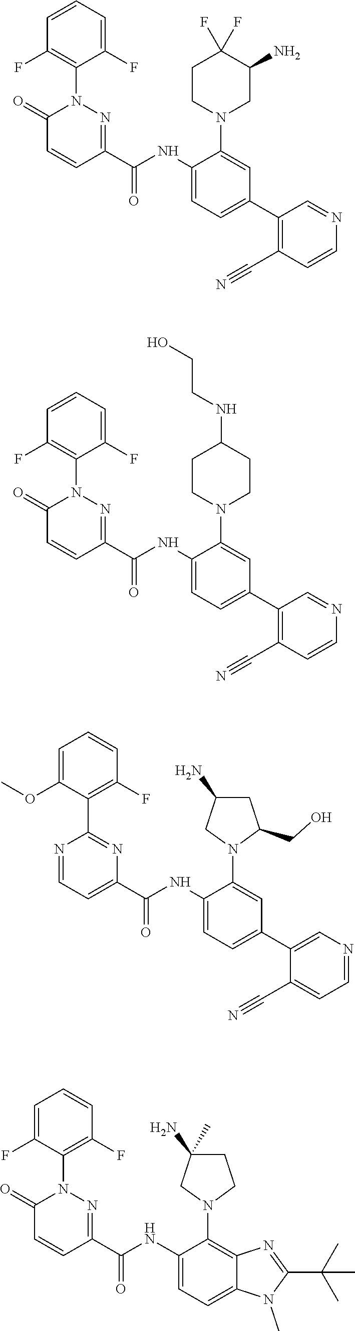 [SMILES: N#Cc1ccncc1-c1ccc(NC(=O)c2ccc(=O)n(-c3c(F)cccc3F)n2)c(N2CCC(F)(F)[C@@H](N)C2)c1 ; N#Cc1ccncc1-c1ccc(NC(=O)c2ccc(=O)n(-c3c(F)cccc3F)n2)c(N2CCC(NCCO)CC2)c1 ; COc1cccc(F)c1-c1nccc(C(=O)Nc2ccc(-c3cnccc3C#N)cc2N2C[C@@H](N)C[C@H]2CO)n1 ; Cn1c(C(C)(C)C)nc2c(N3CC[C@](C)(N)C3)c(NC(=O)c3ccc(=O)n(-c4c(F)cccc4F)n3)ccc21]

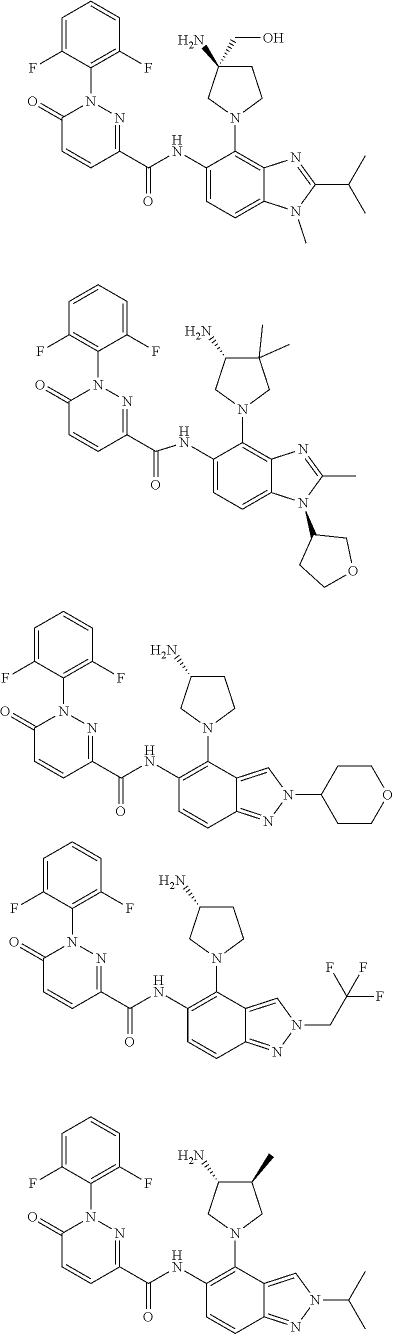

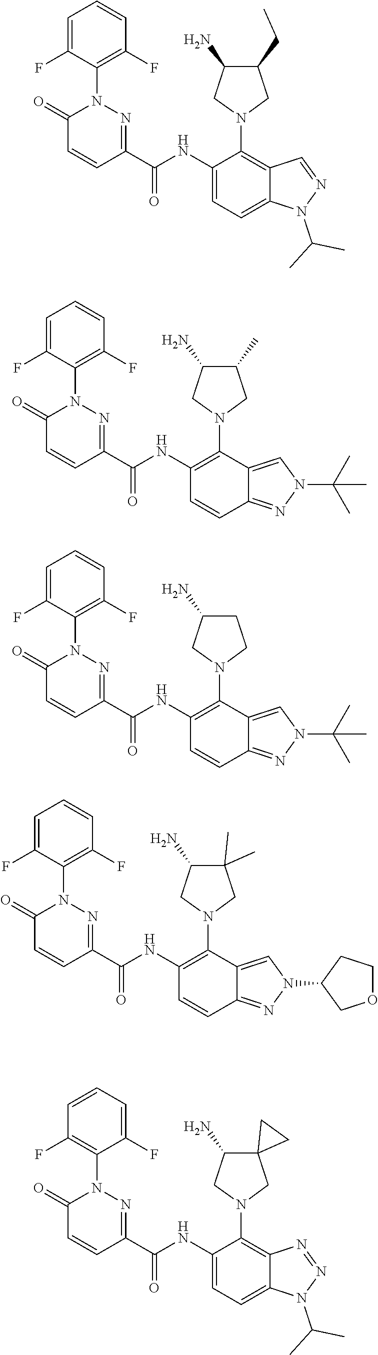 [SMILES: CC[C@H]1CN(c2c(NC(=O)c3ccc(=O)n(-c4c(F)cccc4F)n3)ccc3c2cnn3C(C)C)C[C@H]1N ; C[C@@H]1CN(c2c(NC(=O)c3ccc(=O)n(-c4c(F)cccc4F)n3)ccc3nn(C(C)(C)C)cc23)C[C@@H]1N ; CC(C)(C)n1cc2c(N3CC[C@@H](N)C3)c(NC(=O)c3ccc(=O)n(-c4c(F)cccc4F)n3)ccc2n1 ; CC1(C)CN(c2c(NC(=O)c3ccc(=O)n(-c4c(F)cccc4F)n3)ccc3nn([C@@H]4CCOC4)cc23)C[C@@H]1N ; CC(C)n1nnc2c(N3C[C@H](N)C4(CC4)C3)c(NC(=O)c3ccc(=O)n(-c4c(F)cccc4F)n3)ccc21]

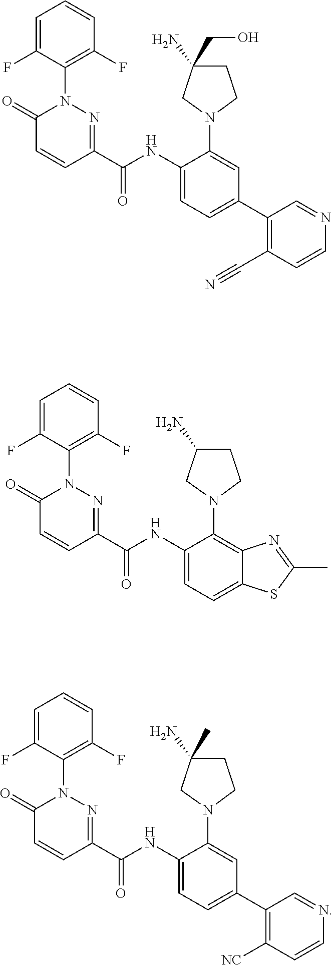 [SMILES: N#Cc1ccncc1-c1ccc(NC(=O)c2ccc(=O)n(-c3c(F)cccc3F)n2)c(N2CC[C@](N)(CO)C2)c1 ; Cc1nc2c(N3CC[C@@H](N)C3)c(NC(=O)c3ccc(=O)n(-c4c(F)cccc4F)n3)ccc2s1 ; C[C@@]1(N)CCN(c2cc(-c3cnccc3C#N)ccc2NC(=O)c2ccc(=O)n(-c3c(F)cccc3F)n2)C1]

In some other embodiments, examples of the compounds of Formula (I) include:

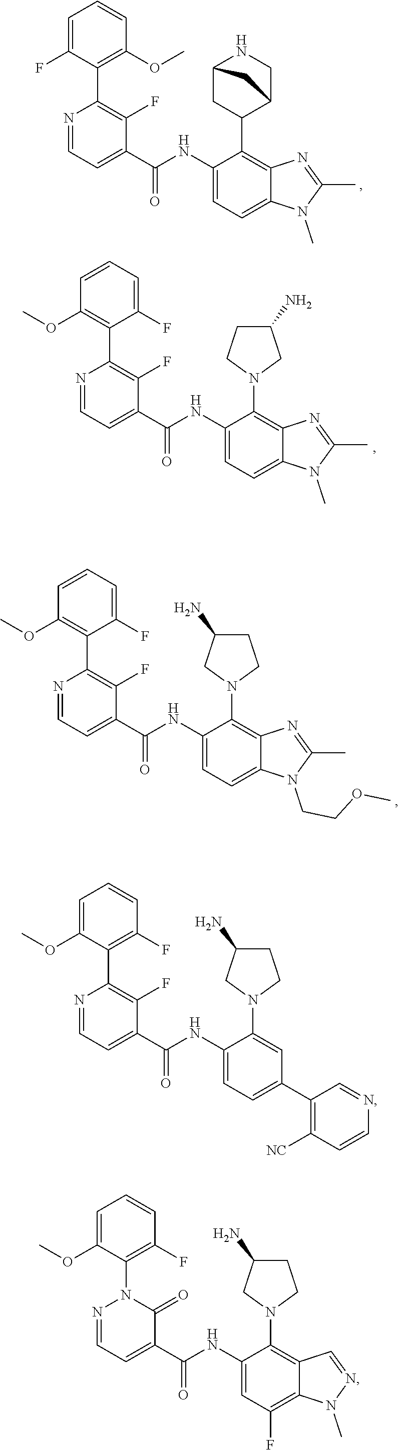

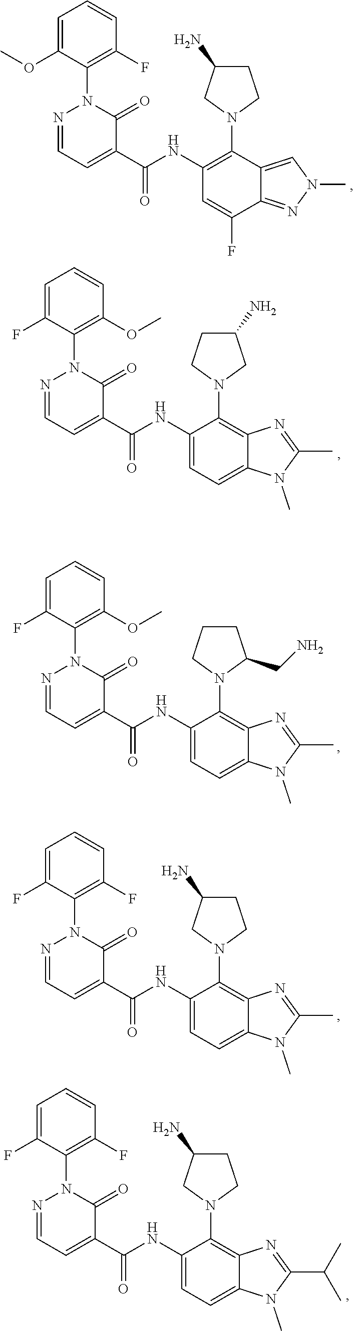

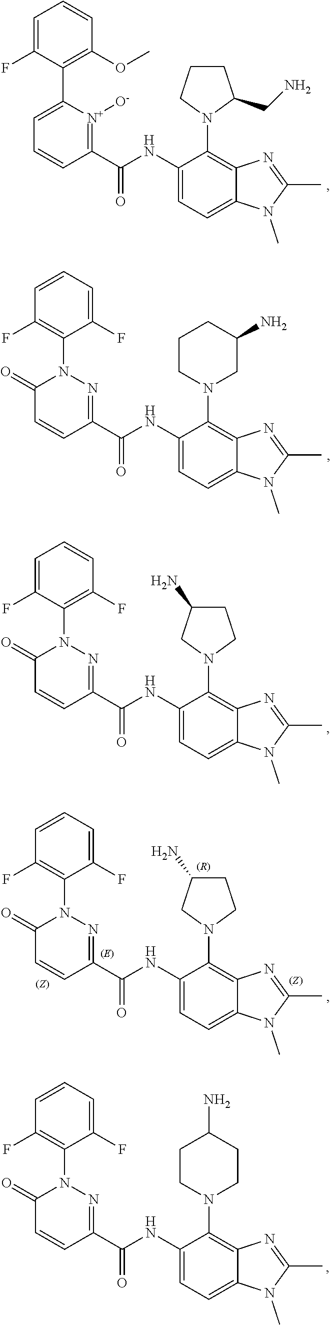

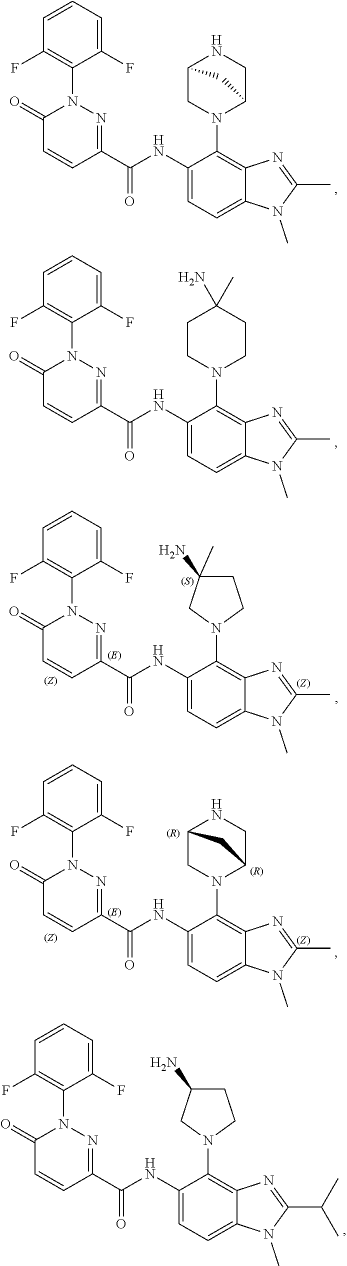

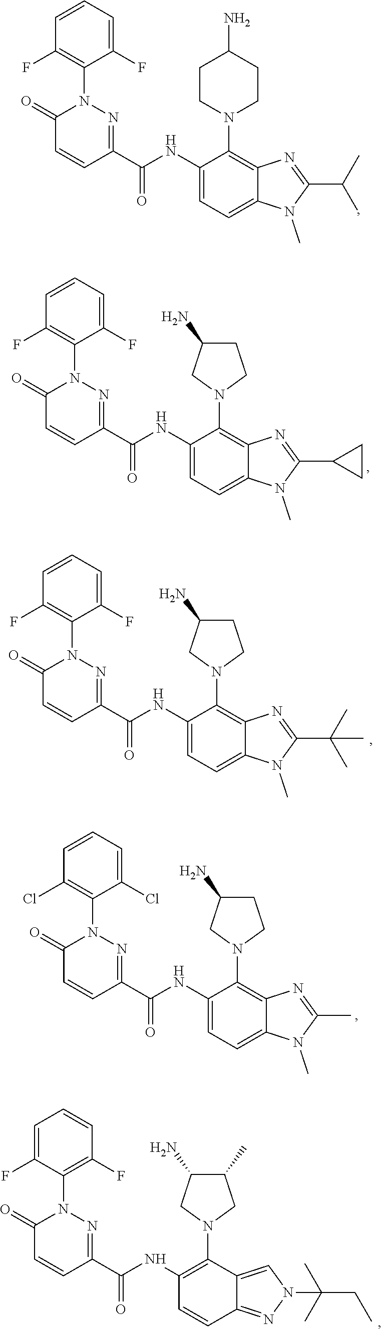

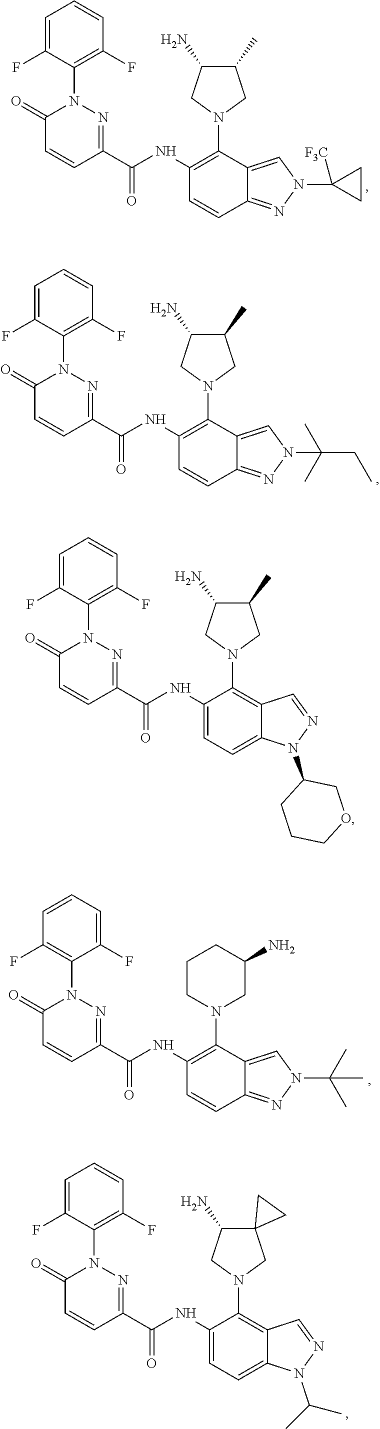 [SMILES: C[C@@H]1CN(c2c(NC(=O)c3ccc(=O)n(-c4c(F)cccc4F)n3)ccc3nn(C4(C(F)(F)F)CC4)cc23)C[C@@H]1N ; CCC(C)(C)n1cc2c(N3C[C@H](C)[C@@H](N)C3)c(NC(=O)c3ccc(=O)n(-c4c(F)cccc4F)n3)ccc2n1 ; C[C@H]1CN(c2c(NC(=O)c3ccc(=O)n(-c4c(F)cccc4F)n3)ccc3c2cnn3[C@@H]2CCCOC2)C[C@@H]1N ; CC(C)(C)n1cc2c(N3CCC[C@@H](N)C3)c(NC(=O)c3ccc(=O)n(-c4c(F)cccc4F)n3)ccc2n1 ; CC(C)n1ncc2c(N3C[C@H](N)C4(CC4)C3)c(NC(=O)c3ccc(=O)n(-c4c(F)cccc4F)n3)ccc21]

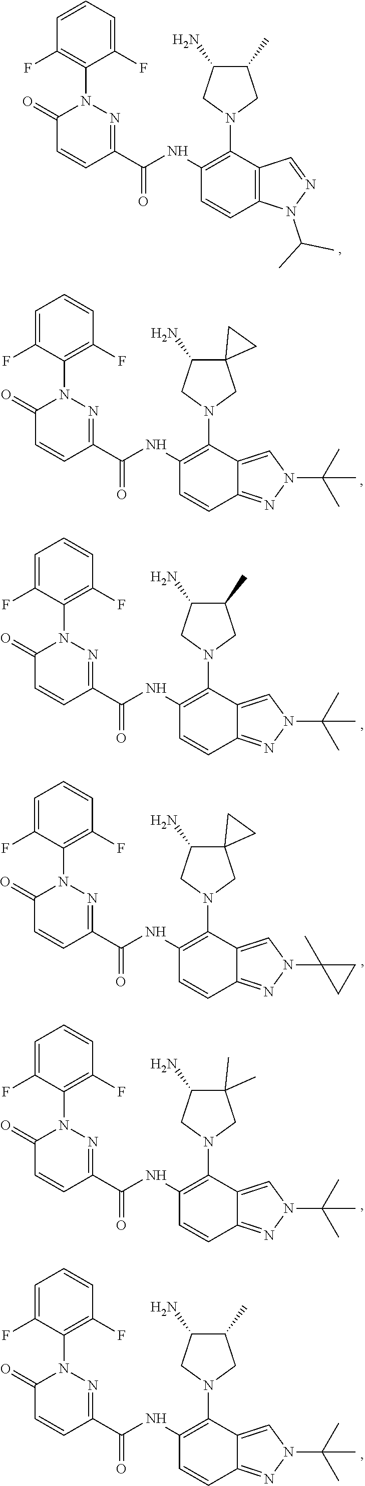 [SMILES: CC(C)n1ncc2c(N3C[C@@H](C)[C@@H](N)C3)c(NC(=O)c3ccc(=O)n(-c4c(F)cccc4F)n3)ccc21 ; CC(C)(C)n1cc2c(N3C[C@H](N)C4(CC4)C3)c(NC(=O)c3ccc(=O)n(-c4c(F)cccc4F)n3)ccc2n1 ; C[C@H]1CN(c2c(NC(=O)c3ccc(=O)n(-c4c(F)cccc4F)n3)ccc3nn(C(C)(C)C)cc23)C[C@@H]1N ; CC1(n2cc3c(N4C[C@H](N)C5(CC5)C4)c(NC(=O)c4ccc(=O)n(-c5c(F)cccc5F)n4)ccc3n2)CC1 ; CC1(C)CN(c2c(NC(=O)c3ccc(=O)n(-c4c(F)cccc4F)n3)ccc3nn(C(C)(C)C)cc23)C[C@@H]1N ; C[C@@H]1CN(c2c(NC(=O)c3ccc(=O)n(-c4c(F)cccc4F)n3)ccc3nn(C(C)(C)C)cc23)C[C@@H]1N]

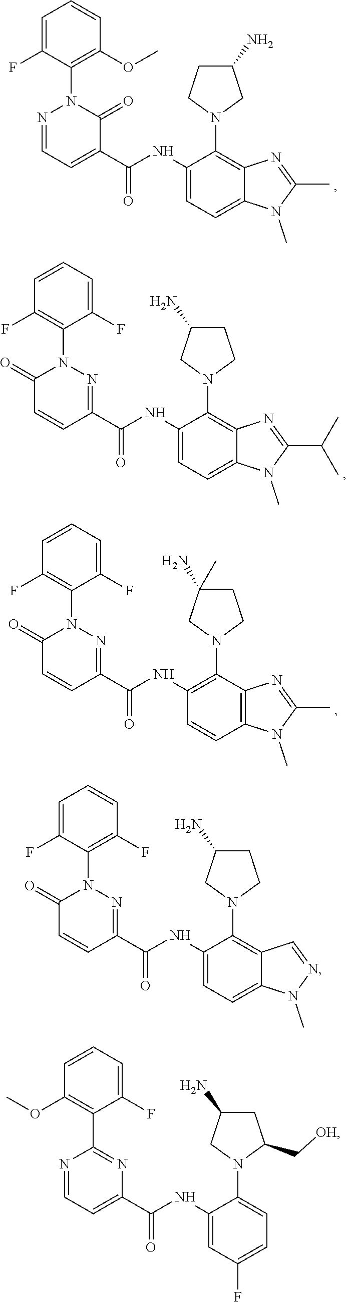

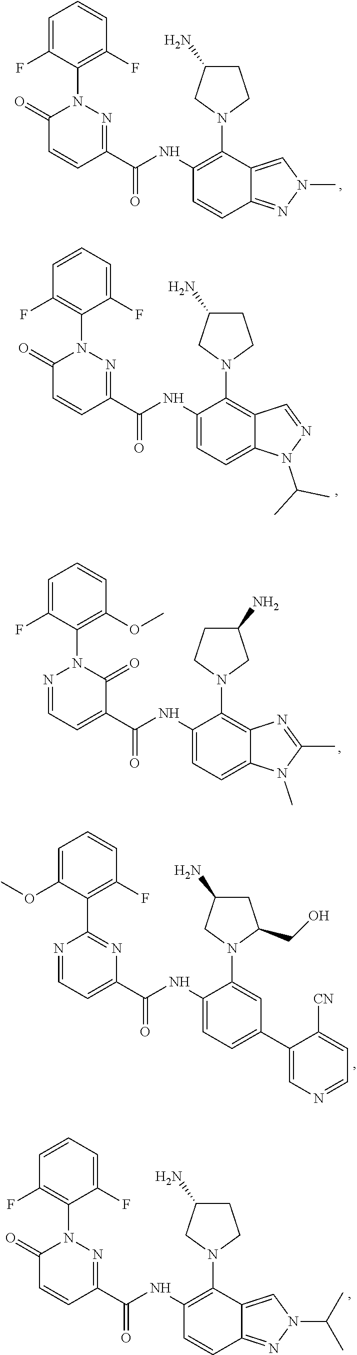

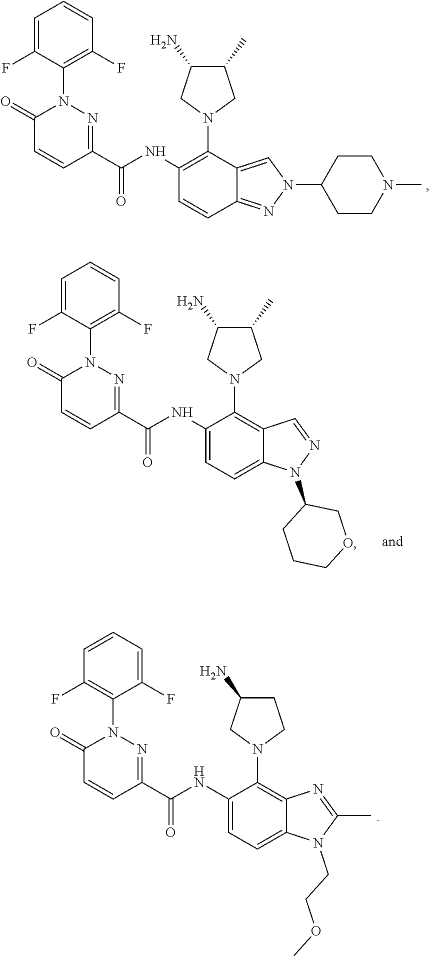 [SMILES: C[C@@H]1CN(c2c(NC(=O)c3ccc(=O)n(-c4c(F)cccc4F)n3)ccc3nn(C4CCN(C)CC4)cc23)C[C@@H]1N ; C[C@@H]1CN(c2c(NC(=O)c3ccc(=O)n(-c4c(F)cccc4F)n3)ccc3c2cnn3[C@@H]2CCCOC2)C[C@@H]1N ; COCCn1c(C)nc2c(N3CC[C@H](N)C3)c(NC(=O)c3ccc(=O)n(-c4c(F)cccc4F)n3)ccc21]

Another aspect of this invention includes pharmaceutical compositions each including a compound as described, and a pharmaceutically acceptable carrier or excipient. Such pharmaceutical composition may include a second therapeutic agent which can be, e.g., an immune-checkpoint inhibitor or an inhibitor of the PGE2 or adenosine pathway. Examples of a suitable immune-checkpoint inhibitor include ipilimumab, nivolumab, and pembroluzimab.

Yet still another aspect of this invention provides a method for treating a disorder mediated by HPK1 in a subject in need thereof. The method includes administering to the subject a therapeutically effective amount of a compound or a pharmaceutical composition as described above. In some embodiments, the disorder is cancer. Cancers that can be treated (including reduction in the likelihood of recurrence) by the methods of the present teachings include breast cancer, colorectal cancer, lung cancer, ovarian cancer, uterine cancer, prostate cancer, leukemias, lymphomas, brain cancer (including glioblastoma multiforme and neuroblastoma), head and neck cancer, pancreatic cancer, melanoma, hepatocellular carcinoma, renal cancer, and soft tissue sarcomas.

### DEFINITIONS

Unless the context indicates otherwise, references to Formula (I) in all sections of this document (including the uses, methods and other aspects of the invention) include references to all other sub-formula, sub-groups, preferences, embodiments and examples as defined herein.

Unless otherwise stated, the following terms used in the specification and claims have the meanings discussed below:

As used herein, the term “unsaturated” or “partially unsaturated” refers to a moiety that includes at least one double or triple bond.

As used herein, the term “saturated” refers to a moiety that does not contain a double or triple bond, i.e., the moiety only contains single bonds.

As used herein, the term “alkyl” refers to a saturated straight (i.e., unbranched) or branched hydrocarbon chain radical consisting of carbon and hydrogen atoms, containing no unsaturation, having the stated number of carbon atoms (e.g., C₁-C₁₀ or C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3 carbon atoms, 4 carbon atoms, etc., up to and including 10 carbon atoms, although the present definition also covers the occurrence of the term “alkyl” where no numerical range is designated. Examples include, but not limited to, methyl, ethyl, propyl, 2-propyl, n-butyl, iso-butyl, tert-butyl, pentyl, hexyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like.

As used herein, the term “substituted alkyl” refers to alkyl substituted with one or more substituents. Examples of the substituent include, but not limited to, halogen, hydroxyl, cyano, amino, alkoxyl, alkoxyalkyl, haloalkyl, alkoxy, amino, methylamino, di-methylamino, sulfone, sulfonamide, aryl, heteroaryl, heterocyclyl, trifluoroethyl, hydroxyethyl, cyanoethyl, methoxyethyl and trifluoropropyl.

The term “alkylene” by itself or as part of another molecule means a divalent radical derived from an alkane, which can be a straight chain or branched chain. In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, refers to an alkylene group having from 1 to 4 carbon atoms.

As used herein, the term “alkoxy” or “alkoxyl” refers to a saturated straight or branched hydrocarbon linked to an oxygen atom. Examples include methoxy, ethoxy, propoxy, 2-propoxy, n-butoxy, iso-butoxy, tert-butoxy, pentoxy, hextoxy, and the like, preferably methoxy, ethoxy, propoxy or 2-propoxy. Representative saturated straight chain alkoxys include methoxy, ethoxyl, n-propoxy, n-butoxy, n-pentoxy, n-hextoxy, and the like; while saturated branched alkoxys include isopropoxyl, sec-butoxy, isobutoxy, tert-butoxy, isopentoxy, and the like. Cyclic alkoxy are referred to herein as a “cycloalkoxy.” “C₁₋₄alkoxy” refers to an alkyl with 1, 2, 3, or 4 carbon atoms. Alkoxy can be linked to a molecule by one or two attachment points.

The term “alkoxyalkyl,” as used herein, refers to an alkyl group substituted with one, two, or three alkoxy groups.

As used herein, the term “alkenyl” refers to an unsaturated straight or branched hydrocarbon. Example include ethenyl and propenyl. The alkenyl can also be substituted with one or more alkyl group(s).

As used herein, the term “alk” (e.g., as a prefix in alkoxyalkyl and alkthioalkyl) refers to a non-cyclic carbon hydron group of alkyl, alkenyl or alkynyl. Unless otherwise specified, the term “alk” encompasses “alkyl,” “alkenyl” and “alkynyl.”

As used herein, the term a “carbonyl group” refers to —C(═O)—.

As used herein, the term “cycloalkyl” by itself or as part of another substituent refers to a non-aromatic carbon-based ring composed of at least three carbon atoms. The term cycloalkyl includes monocyclic cycloalkyl, bicyclic cycloalkyl, polycyclic cycloalkyl, bridged cycloalkyl, fused cycloalkyl, and spiro cycloalkyl groups. In a bridged cycloalkyl, the rings share at least two common non-adjacent atoms. In a fused bicyclic cycloalkyl, two rings share a covalent bond. In a spirocyclic cycloalkyl group, one atom is common to two different rings.

The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted.

The term “multicycloalkyl” refers to cycloalkyl with plural rings that are fused.

As used herein, the term “halo” or “halogen” refers to fluorine (fluoro, —F), chlorine (chloro, —Cl), bromine (bromo, —Br), or iodine (iodo, —I). “Haloalkyl” refers to alkyl as defined above in which one or more of the hydrogen atoms have been replaced with a halogen independently selected from fluoro, chloro, bromo, and iodo. “Fluoroalkyl” means alkyl as defined above wherein one or more hydrogen atoms have been replaced by fluoro atoms. Unless otherwise specified with a number, a haloalkyl can include as many as chemically possible halo atoms as substituents on the alkyl group. For example, fluoroethyl can be —CH₂CF₃, —CHF—CH₃, or —CH₂CH₂F.

As used herein, the term “hydrogen” (or H) includes its isotopes of deuterium (D or ²H) and tritium (³H), meaning a or any hydrogen atom in the compounds of this invention can be replaced with either deuterium (D or ²H) and tritium (³H).

As used herein, the term “hydroxyl” or “hydroxy” refers to the group —OH.

The term “hydroxyalkyl” by itself or as part of another substituent refers to an alkyl group in which one or more of the hydrogen atoms are replaced with a hydroxyl substituent. Thus, the term “hydroxyalkyl” includes monohydroxyalkyls, dihydroxyalkyls, trihydroxyalkyls, etc.

The term “cyano” as used herein refers to a group of —C≡N. The term “cyanoalkyl” as used herein, refers to an alkyl group having at least one —CN substituent.

As used herein, the term “carbonyl” refers to a —C(═O)— group.

As used herein, the term “amino” or “amine” as used herein refers to —NH₂. The term “alkylamino” refers to a group of the formula —NHR, and “dialkylamino” refers to a group of the formula —NRR, where each R is independently an alkyl.

The term “amido” or “amide” indicates either a C-amido group such as —C(═O)NR′R″ or an N-amido group such as —NR′C(═O)R″, wherein R′ or R″ is substitution group. Examples of R′ and R″ include but not limited to hydrogen, alkyl, alkenyl, alkynyl, alkoxy, alkoxyalkyl, hydroxy, hydroxyalkyl, hydroxyalkamino, amino, alkamino, cycloalkyl, alk-cycloalkyl, aryl, alkaryl, heterocycloalkyl, alk-heterocycloalkyl, heteroaryl and alkheteroaryl.

As used herein, the term “nitro,” as used herein, refers to —NO₂.

As used herein, the term “alkylthioalkyl” or “alkthioalkyl” refers to the group -alkyl-S-alkyl, wherein “alk” is an alkylene group. In general, if a compound is attached to an “alkthioalkyl” group, the alkylene portion of the “alkthioalkyl” group is attached to the compound.

As used herein, the term “alk-S(═O)₂-alkyl” or “alkyl-S(═O)₂-alkyl” refers to the group -alkyl-S(═O)₂-alkyl, wherein “alk” is an alkylene group.

As used herein, the term “alkamino” refers to an amino group, described herein, that is attached to an alkylene. In general, if a compound is attached to an alkamino group, the alkylene portion of the alkamino is attached to the compound.

The term “alk-cycloalkyl” refers to a cycloalkyl group that is connected to an alkylene. In general, if a compound is attached to an alk-cycloalkyl group, the alkylene portion of the alk-cycloalkyl group is attached to the compound.

As used herein, the term “aromatics” or “aromatic ring” or similar terms refer to planar rings having a delocalized pi-electron system containing 4n+2 pi-electrons, where n is a positive integer. Aromatic rings can be formed from five, six, seven, eight, nine, ten or more than ten atoms. Aromatics are optionally substituted. The term “aromatic” includes carbocyclic aryl (“aryl”, e.g., phenyl) and “heteroaryl” (or “heteroaromatic”) groups (e.g., pyridine). The term includes monocyclic or fused-ring polycyclic (i.e., rings that share adjacent pairs of carbon atoms) groups.

A dashed bond represents a single bond or a double bond as required to complete the valencies of the atoms being linked by the bond. It will be understood that in some instances the bond has aromatic character.

As used herein, the term “aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl.

As used herein, the term “heteroaryl” refers to a monocyclic or fused ring (i.e., rings which share an adjacent pair of atoms) of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, carbazole, benzimidazole, benzoxazole, benzthiazole, indazole and quinazoline. The heteroaryl group may be substituted or unsubstituted.

The above-defined groups may include prefixes and/or suffixes that are commonly used in the art to create additional well-recognized substituent groups. As examples, the term “haloalkoxy” or “haloalkyloxy” refers to a haloalkyl group attached to the parent molecular moiety through an oxygen atom. The term “(haloalkyl)oxyalkyl” refers to an alkyl group substituted with one, two, or three (haloalkyl)oxy groups. As another example, the term “hydroxyalkamino” refers to an amino group substituted with one or two hydroxyalkyl groups.

Stereoisomers are compounds which differ only in their spatial arrangement. When a disclosed compound is named or depicted by structure without indicating stereochemistry, it is understood that the name or structure encompasses all possible stereoisomers, geometric isomers, including essentially pure stereo or geometric isomers, as well as combination thereof.

A combination of substituents is permissible only if such as combination results in a stable or chemically feasible compound (i.e., one that is not substantially altered when kept at 40° C. or less for at least a week).

The term “optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

A “pharmaceutically acceptable salt,” prepared by acid addition, is one formed from an acid which then forms a non-toxic acid anion such as the hydrochloride, hydrobromide, sulphate, phosphate or acid phosphate, acetate, maleate, fumarate, lactate, tartrate, citrate and gluconate salt. It will be understood that, as used herein, references to the compounds of formula (I) are meant to also include the pharmaceutically acceptable salts.

When a compound the present invention has a carboxy group, it can be made to a pharmaceutically acceptable ester in an ordinary method (e.g., condensation reaction of a carboxylic acid with an alcohol), by reacting the compound with a corresponding alcohol (e.g., C₁ alcohol).

A “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

The term “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The term “therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

As used herein, the term “subject” or “patient” is used interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

The compounds taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

### Isomeric Forms

The present invention provides compounds of Formula (I) or pharmaceutically acceptable salts, esters, or prodrugs thereof, useful as HPK1 inhibitors, and methods of use thereof.

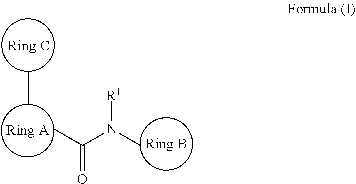

Certain compounds of Formula (I) (or salts, prodrugs, or conjugates) may exist in, and be isolated in, isomeric forms, including tautomeric forms, geometric isomers (i.e., cis- or trans-isomers), optical isomers (i.e., enantiomers and diastereomers), racemic forms, or any mixture of the isomeric forms described above. It is to be understood that the present invention encompasses a compound of formula (I) in any of the isomeric forms or as a mixture thereof, for example, in the form of an active single enantiomer, racemic, or any mixture thereof. The present invention is meant to comprehend all such isomeric forms of the compounds of Formula (I).

In addition, a compound of formula (I) (or salt, prodrug or conjugate thereof) may exhibit polymorphism or may form a solvate with water or an organic solvent. The present invention also encompasses any such polymorphic form, any solvate or any mixture thereof.

### Example 1: N-(2-((1S,4S)-2,5-diazabicyclo[2.2.1]heptan-2-yl)-5-fluorophenyl)-3-fluoro-2-(2-fluoro-6-methoxyphenyl)isonicotinamide

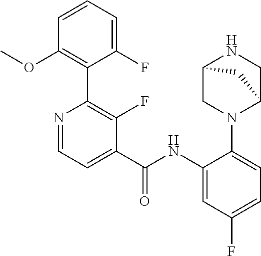 [SMILES: COc1cccc(F)c1-c1nccc(C(=O)Nc2cc(F)ccc2N2C[C@@H]3C[C@H]2CN3)c1F]

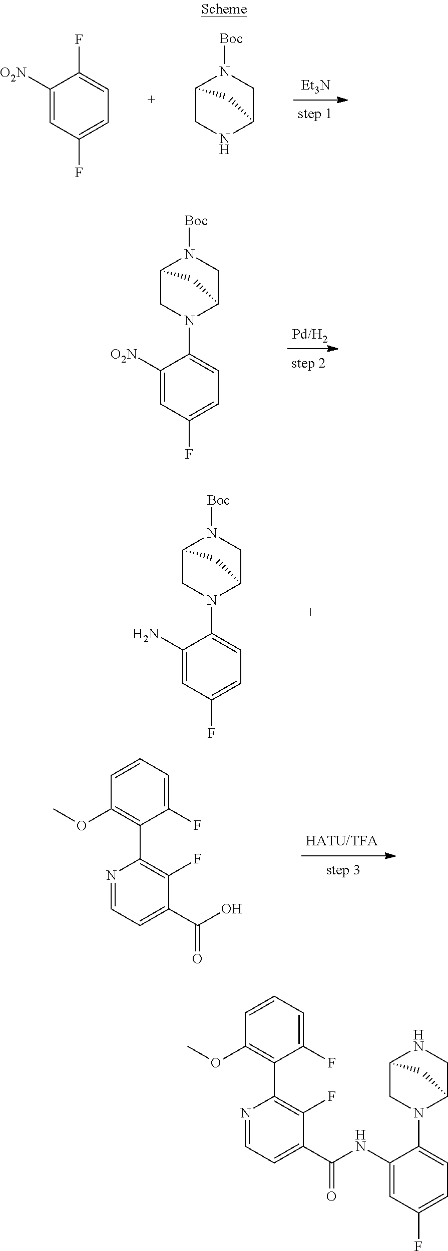

### Step1: tert-butyl (1S,4S)-5-(4-fluoro-2-nitrophenyl)-2,5-diazabicyclo[2.2.1]heptane-2-carboxylate

A mixture of 1,4-difluoro-2-nitrobenzene (0.85 g 5.4 mmol 1.00 eq), tert-butyl (1S,4S)-2,5-diazabicyclo[2.2.1]heptane-2-carboxylate (1.00 g 5.4 mmol 1.00 eq), Et₃N (1 mL) in ACN (15 mL) was stirred at 60° C. for 10 h. TLC showed the reaction finished. The mixture was poured into water 100 mL, was extracted with EA 100 mL (twice). The organic layers were dried over Na₂SO₄ and concentrated to dryness in vacuo affording tert-butyl (1S,4S)-5-(4-fluoro-2-nitrophenyl)-2,5-diazabicyclo[2.2.1]heptane-2-carboxylate (2.5 g).

LC-MS m/z: 338.3 (M+1)⁺.

### Step 2: tert-butyl (1S,4S)-5-(2-amino-4-fluorophenyl)-2,5-diazabicyclo[2.2.1]heptane-2-carboxylate

To a solution of tert-butyl (1S,4S)-5-(4-fluoro-2-nitrophenyl)-2,5-diazabicyclo[2.2.1]heptane-2-carboxylate 2.5 g in MeOH 30 mL, was added Pd/C 0.25 g under hydrogen for 15 h. LCMS showed the reaction finished. The mixture of reaction was filtered, the filtrate was concentrated to dryness in vacuo affording tert-butyl (1S,4S)-5-(2-amino-4-fluorophenyl)-2,5-diazabicyclo[2.2.1]heptane-2-carboxylate (1.32 g) as a white solid.

LC-MS m/z: 308.2 (M+1)⁺.

### Step 3: N-(2-((1S,4S)-2,5-diazabicyclo[2.2.1]heptan-2-yl)-5-fluorophenyl)-3-fluoro-2-(2-fluoro-6-methoxyphenyl)isonicotinamide

To a solution of tert-butyl (1S,4S)-5-(2-amino-4-fluorophenyl)-2,5-diazabicyclo[2.2.1]heptane-2-carboxylate (95 mg 0.36 mmol 1.00 eq) and DCM 5 mL, was added 3-fluoro-2-(2-fluoro-6-methoxyphenyl)isonicotinic acid (111 mg 0.36 mmol 1.00 eq) and HATU (274 mg 0.72 mmol 2.00 eq), stirred at the room temperature (RT) for 4 hours. LCMS showed target compound MS and the reaction finished. The mixture was poured into water 30 mL, was exacted with EA 30 mLx2, The organic layers was dried over Na₂SO₄ and concentrated to dryness in vacuo. The resulting residue was purified by column chromatography (EA:PE=1:1) affording 58 mg as a white solid, was added with CF₃COOH 1 mL and DCM 1 mL, stirred at RT for 2 h, was concentrated to dryness in vacuo affording Example 1 (30 mg) as a white solid.

LC-MS m/z: 455.6 (M+1)⁺

¹H NMR (400 MHz, CDCl₃) δ 10.05 (s, 1H), 9.72 (dd, J=29.5, 13.0 Hz, 1H), 8.79 (d, J=4.9 Hz, 1H), 8.46-8.34 (m, 1H), 8.15-8.04 (m, 1H), 7.72-7.58 (m, 1H), 7.50-7.39 (m, 1H), 6.97-6.81 (m, 3H), 4.33 (s, 1H), 3.87 (s, 1H), 3.84 (s, 3H), 3.78 (dd, J=11.5, 5.1 Hz, 1H), 3.56 (d, J=10.3 Hz, 1H), 3.43 (t, J=10.7 Hz, 1H), 3.13 (s, 1H), 2.36 (d, J=10.9 Hz, 1H), 2.20 (t, J=9.7 Hz, 1H).

| No. | Structure | Salt form | MS and ¹HNMR |
|---|---|---|---|
| Example 2 | 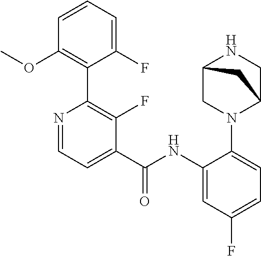 [SMILES: COc1cccc(F)c1-c1nccc(C(=O)Nc2cc(F)ccc2N2C[C@H]3C[C@@H]2CN3)c1F] | TFA Salt | LC-MS m/z: 455.6 (M + 1)⁺ ¹H NMR (400 MHz, CDCl₃) δ 10.03 (s, 2H), 9.73 (dd, J = 29.9, 13.3 Hz, 1H), 8.78 (t, J = 8.8 Hz, 1H), 8.40 (ddd, J = 9.5, 6.3, 2.9 Hz, 1H), 8.19-8.03 (m, 1H), 7.64 (dd, J = 8.9, 5.3 Hz, 1H), 7.47 (tt, J = 19.0, 9.5 Hz, 1H), 6.96-6.77 (m, 3H), 4.33 (s, 1H), 3.89 (d, J = 15.3 Hz, 1H), 3.84 (s, 3H), 3.81- 3.74 (m, 1H), 3.56 (d, J = 9.7 Hz, 1H), 3.43 (t, J = 11.4 Hz, 1H), 3.12 (s, 1H), 2.36 (d, J = 10.8 Hz, 1H), 2.20 (t, J = 9.8 Hz, 1H). |
| | N-(2-((1R,4R)-2,5- | | |
| | diazabicyclo[2.2.1]heptan- | | |
| | 2-yl)-5-fluorophenyl)-3- | | |
| | fluoro-2-(2-fluoro-6- | | |
| | methoxyphenyl)isonicotina- | | |
| | mide | | |

### Example 3: N-(2-((1R,4R)-2,5-diazabicyclo[2.2.1]heptan-2-yl)-5-fluorophenyl)-2,2′-difluoro-6′-methoxy-[1,1′-biphenyl]-3-carboxamide

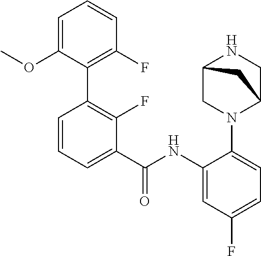 [SMILES: COc1cccc(F)c1-c1cccc(C(=O)Nc2cc(F)ccc2N2C[C@H]3C[C@@H]2CN3)c1F]

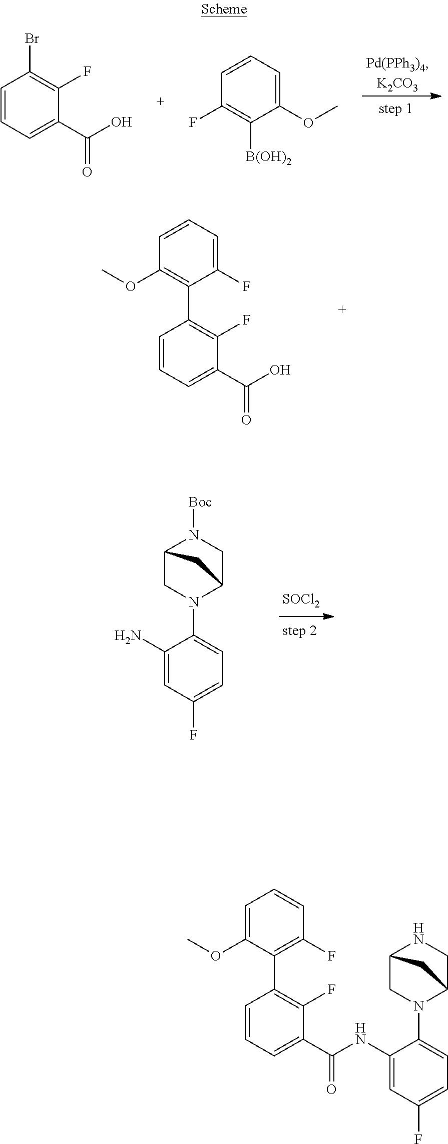

### Step 1: 2,2′-difluoro-6′-methoxy-[1,1′-biphenyl]-3-carboxylic acid

A mixture of 3-bromo-2-fluorobenzoic acid (500 mg 2.27 mmol 1.00 eq), (2-fluoro-6-methoxyphenyl)boronic acid (772 mg 4.54 mmol 2.00 eq), Pd(PPh₃)₄ (127 mg 0.11 mmol 0.05 eq), K₂CO₃ (941 mg 6.81 mmol 3.00 eq) 1,4-dioxane 5 mL, EtOH 5 mL and water 1 mL was stirred at 100° C. for 2 h in microwave. LCMS analysis of the reaction mixture showed full conversion to the desired product. The mixture was poured into water 40 mL and AcOH 2 mL, was exacted with EA 40 mLx2. The organic layer was dried over MgSO₄ and concentrated to dryness in vacuo. The resulting residue was purified by column chromatography (EA:MeOH=3:1) affording 2,2′-difluoro-6′-methoxy-[1,1′-biphenyl]-3-carboxylic acid (135 mg, yield 23%) as a white solid. LC-MS m/z: 263.2 (M−1)⁻.

### Step 2: N-(2-((1R,4R)-2,5-diazabicyclo[2.2.1]heptan-2-yl)-5-fluorophenyl)-2,2′-difluoro-6′-methoxy-[1,1′-biphenyl]-3-carboxamide

To a solution of 2,2′-difluoro-6′-methoxy-[1,1′-biphenyl]-3-carboxylic acid (50 mg 0.19 mmol 1.00 eq) in toluene 10 mL, was added SOCl₂ 2 mL, was stirred at 80° C. for 2 h, then concentrated to dryness in vacuo. The residue was added DCM 5 mL, Et₃N 2 mL, and tert-butyl (1R,4R)-5-(2-amino-4-fluorophenyl)-2,5-diazabicyclo[2.2.1]heptane-2-carboxylate (58 mg 0.19 mol 1.1 eq), was stirred at RT for 3 h, LCMS analysis of the reaction mixture showed full conversion to the desired product. The reaction mixture was poured into water 30 mL, was exacted with EA 30 mLx2. The organic layer was dried over Na₂SO₄ and concentrated to dryness in vacuo. The resulting residue was purified by column chromatography (EA:PE=1:1) affording 30 mg as white solid, then was added CF₃COOH 1 mL and DCM 1 mL, stirred at RT for 2 h, was concentrated to dryness in vacuo, then was added ammonium hydroxide 3 mL and water 10 mL, exacted with EA 10 mL, concentrated to dryness in vacuo affording Example 3 (7 mg, Yield: 8%) as a white solid.

LC-MS m/z: 454.5 (M+1)⁺.

¹H NMR (400 MHz, CDCl₃) δ 9.79 (dd, J=31.1, 14.7 Hz, 1H), 8.60 (s, 1H), 8.41 (ddd, J=10.6, 7.8, 2.9 Hz, 1H), 8.31-8.13 (m, 1H), 7.63-7.54 (m, 1H), 7.50 (ddd, J=8.6, 5.3, 3.1 Hz, 1H), 7.45-7.36 (m, 2H), 6.95-6.79 (m, 3H), 4.13 (s, 1H), 3.83 (s, 3H), 3.81 (s, 1H), 3.65 (dd, J=10.6, 3.5 Hz, 1H), 3.45-3.30 (m, 2H), 3.01 (d, J=10.9 Hz, 1H), 2.24 (d, J=10.3 Hz, 1H), 2.05 (t, J=9.9 Hz, 1H).

### Example 4: N-(2-((1R,4R)-2,5-diazabicyclo[2.2.1]heptan-2-yl)-5-fluorophenyl)-2-(3-chloro-2-fluoro-6-methoxyphenyl)-3-fluoroisonicotinamide

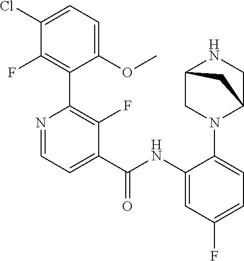 [SMILES: COc1ccc(Cl)c(F)c1-c1nccc(C(=O)Nc2cc(F)ccc2N2C[C@H]3C[C@@H]2CN3)c1F]

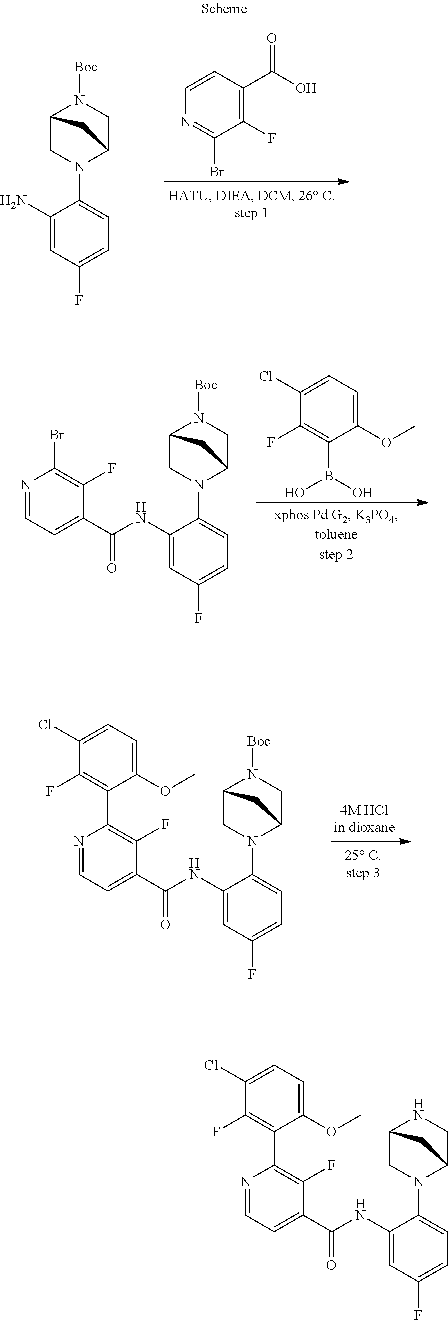

### Step 1: tert-butyl (1R,4R)-5-(2-(2-bromo-3-fluoroisonicotinamido)-4-fluorophenyl)-2,5-diazabicyclo[2.2.1]heptane-2-carboxylate

To a solution of 2-bromo-3-fluoroisonicotinic acid (429.45 mg, 1.95 mmol, 1.0 eq.) in DCM (10 mL) was added HATU (1.11 g, 2.93 mmol, 1.5 eq.) and DIEA (504 mg, 3.90 mmol, 2.0 eq.) was stirred for 30 min at 26° C., then the tert-butyl (1R,4R)-5-(2-amino-4-fluorophenyl)-2,5-diazabicyclo[2.2.1]heptane-2-carboxylate (600 mg, 1.95 mmol, 1.0 eq.) was added. The reaction was stirred at 26° C. overnight to give a brown solution. LCMS showed one of the major peak with the desired mass and the material was almost consumed. The reaction was washed by water and extracted with DCM. The organic phase was concentrated under reduced pressure to dryness to give compound tert-butyl (1R,4R)-5-(2-(2-bromo-3-fluoroisonicotinamido)-4-fluorophenyl)-2,5-diazabicyclo[2.2.1]heptane-2-carboxy late (0.71 g, Yield: 71.4%) as a black-brown oil liquid.

LC-MS m/z: 509.1 (M+1)⁺.

### Step 2: tert-butyl (1R,4R)-5-(2-(2-(3-chloro-2-fluoro-6-methoxyphenyl)-3-fluoroisonicotinamido)-4-fluorophenyl)-2,5-diazabicyclo[2.2.1]heptane-2-carboxylate

To a suspension of tert-butyl (1R,4R)-5-(2-(2-bromo-3-fluoroisonicotinamido)-4-fluorophenyl)-2,5-diazabicyclo[2.2.1]heptane-2-carboxylate (100 mg, 196.33 umol, 1.0 eq.) in toluene (2 mL) was added (3-chloro-2-fluoro-6-methoxyphenyl)boronic acid (120.38 mg, 588.98 umol, 3.0 eq), K₃PO₄ (83.35 mg, 392.66 umol, 2.0 eq) and xphos Pd G₂ (15.43 mg, 19.633 umol, 0.1 eq). The reaction was stirred for 2 h at 100° C. to give a brown suspension. LCMS showed the major peak with the desired mass and the material was still remained. The reaction was washed by water and extracted with DCM. The organic phase was concentrated under reduced pressure to dryness and purified by flash column (SiO₂, eluted with 0^{˜}30% EA in PE, COMBI FLASH NEXTAGEN 300) to give tert-butyl (1R,4R)-5-(2-(2-(3-chloro-2-fluoro-6-methoxyphenyl)-3-fluoroisonicotinamido)-4-fluorophenyl)-2,5-diazabicyclo[2.2.1]heptane-2-carboxylate (102 mg, Yield: 88%) as a light yellow oil.

LC-MS m/z:589.2 (M+1)⁺.

### Step 3: N-(2-((1R,4R)-2,5-diazabicyclo[2.2.1]heptan-2-yl)-5-fluorophenyl)-2-(3-chloro-2-fluoro-6-methoxyphenyl)-3-fluoroisonicotinamide

A mixture of tert-butyl (1R,4R)-5-(2-(2-(3-chloro-2-fluoro-6-methoxyphenyl)-3-fluoroisonicotinamido)-4-fluorophenyl)-2,5-diazabicyclo[2.2.1]heptane-2-carboxylate (102 mg, 173.17 umol, 1.0 eq.) in 4 M HCl in dioxane (2 mL) was stirred overnight at 25° C. to give a yellow solution. LCMS showed one of peak with the desired mass and the material was almost consumed. The reaction was concentrated under reduced pressure to dryness and purified by flash column (C₁₈, peak time=50% ACN in H₂O, COMBI FLASH NEXTAGEN 300) to give Example 4 (30.1 mg, Yield: 35.5%) as a yellow solid.

LC-MS m/z: 489.1 (M+1)⁺.

¹H NMR (400 MHz, CD₃OD): 5 8.70 (d, J=4.9 Hz, 1H), 7.99 (in, J=10.0, 5.0 Hz, 1H), 7.88 (in, J=10.0, 3.3 Hz, 1H), 7.64 (t, J=8.8 Hz, 1H), 7.24 (in, J=9.0, 5.3 Hz, 1H), 7.11-6.96 (m, 2H), 4.43 (s, 1H), 4.19 (s, 1H), 3.84 (s, 3H), 3.55 (in, J=23.1, 11.3 Hz, 2H), 3.40 (d, J=11.5 Hz, 1H), 3.28 (in, J=11.5, 2.2 Hz, 1H), 2.34 (d, J=11.4 Hz, 1H), 2.00 (d, J=11.5 Hz, 1H).

| No. | Structure | Salt form | MS and ¹HNMR |
|---|---|---|---|
| Example 5 | 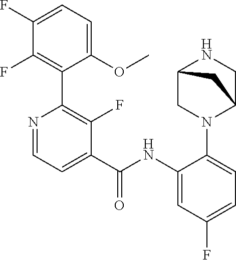 [SMILES: COc1ccc(F)c(F)c1-c1nccc(C(=O)Nc2cc(F)ccc2N2C[C@H]3C[C@@H]2CN3)c1F] | TFA Salt | LC-MS m/z: 473.4 (M + 1)⁺ 1H NMR (400 MHz, CDCl₃) δ 10.10 (s, 2H), 9.71 (t, J = 13.2 Hz, 1H), 8.79 (d, J = 4.9 Hz, 1H), 8.46-8.35 (m, 1H), 8.17-8.08 (m, 1H), 7.64 (dd, J = 8.9, 5.3 Hz, 1H), 7.32 (t, J = 7.0 Hz, 1H), 6.92 (td, J = 8.4, 2.9 Hz, 1H), 6.81-6.75 (m, 1H), 4.38 (d, J = 23.5 Hz, 1H), 3.89 (d, J = 18.0 Hz, 1H), 3.80 (t, J = 5.0 Hz, 3H), 3.78 (d, J = 3.2 Hz, 1H), 3.56 (d, J = 12.1 Hz, 1H), 3.49-3.37 (m, 1H), 3.15 (s, 1H), 2.35 (d, J = 11.0 Hz, 1H), 2.22 (t, J = 10.2 Hz, 1H). |
| | N-(2-((1R,4R)-2,5- | | |
| | diazabicyclo[2.2.1]heptan- | | |
| | 2-yl)-5-fluorophenyl)-2- | | |
| | (2,3-difluoro-6- | | |
| | methoxyphenyl)-3- | | |
| | fluoroisonicotinamide | | |
| Example 6 | 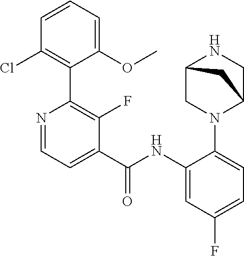 [SMILES: COc1cccc(Cl)c1-c1nccc(C(=O)Nc2cc(F)ccc2N2C[C@H]3C[C@@H]2CN3)c1F] | TFA Salt | LC-MS m/z: 471.2 (M + 1)⁺ ¹H NMR (400 MHz, CDCl₃) δ 10.03 (s, 2H), 9.77-9.62 (m, 1H), 8.79 (d, J = 4.9 Hz, 1H), 8.40 (ddd, J = 9.6, 6.4, 2.9 Hz, 1H), 8.13 (dt, J = 11.0, 5.3 Hz, 1H), 7.71-7.57 (m, 1H), 7.44 (t, J = 8.3 Hz, 1H), 7.18 (dd, J = 7.9, 3.4 Hz, 1H), 7.00 (dt, J = 8.4, 4.3 Hz, 1H), 6.91 (td, J = 8.4, 2.9 Hz, 1H), 4.32 (s, 1H), 3.86 (d, J = 5.3 Hz, 1H), 3.80 (d, J = 2.0 Hz, 3H), 3.76 (d, J = 6.3 Hz, 1H), 3.55 (s, 1H), 3.43 (t, J = 9.5 Hz, 1H), 3.12 (s, 1H), 2.35 (d, J = 11.1 Hz, 1H), 2.23-2.11 (m, 1H). |
| | N-(2-((1R,4R)-2,5- | | |
| | diazabicyclo[2.2.1]heptan- | | |
| | 2-yl)-5-fluorophenyl)-2-(2- | | |
| | chloro-6-methoxyphenyl)- | | |
| | 3-fluoroisonicotinamide | | |

### Example 7: N-(4-((1S,4S)-2-azabicyclo[2.2.1]heptan-5-yl)-1,2-dimethyl-1H-benzo[d]imidazol-5-yl)-3-fluoro-2-(2-fluoro-6-methoxyphenyl)isonicotinamide

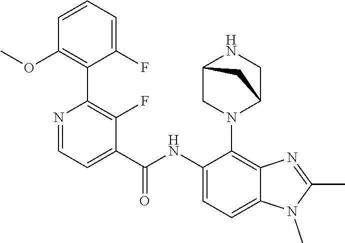

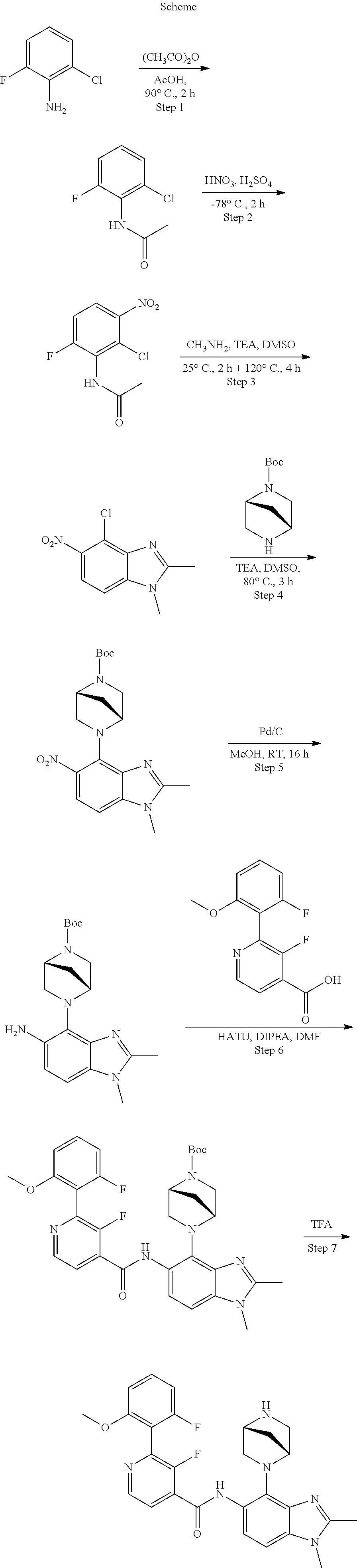

### Step 1: N-(2-chloro-6-fluorophenyl)acetamide

To a solution of 2-chloro-6-fluoroaniline (10 g, 68.7 mmol, 1 eq) in AcOH (40 mL) was added acetic anhydride. The reaction was stirred at 90° C. for 2 hrs. LCMS analysis of the reaction mixture showed full conversion to the desired product. After cooling to R.T, water was added and the solid precipitate was collected by filtration, washed with water, and air dried to afford N-(2-chloro-6-fluorophenyl) acetamide (9.28 g, yield: 72.01%) as a white solid.

LC-MS m/z: 188.1 (M+1)⁺.

### Step 2: N-(2-chloro-6-fluoro-3-nitrophenyl)acetamide

A mixture of N-(2-chloro-6-fluorophenyl)acetamide (9.28 g, 49.47 mmol, 1 eq.) in H₂SO₄ (43 mL) was cooled to −78° C. in a reaction bath and added HNO₃ (2.67 mL, 64.31 mmol, 1.3 eq) dropwise. The mixture was stirred for 2 hrs. LCMS analysis showed that desired mass was detected and reaction was completely. It was then carefully poured into an ice/water mixture, and the solid precipitate was collected by filtration, washed with water, and air dried to afford N-(2-chloro-6-fluoro-3-nitrophenyl)acetamide (16.09 g, Yield: 139.8%) as an off-white solid.

LC-MS m/z: 233.1 (M+1)⁺.

### Step 3: 4-chloro-1,2-dimethyl-5-nitro-1H-benzo[d]imidazole

A mixture of N-(2-chloro-6-fluoro-3-nitrophenyl)acetamide (5.0 g, 21.5 mmol, 1 eq.), a 33% solution of CH₃NH₂ in EtOH (3.37 mL, 21.5 mmol, 1 eq.), and TEA (6 mL, 43 mmol, 1 eq.) in DMSO (20 mL) was stirred at R.T. for 2 hrs. The mixture was then sparged with N₂ for 30 mins, additional TEA (6 mL, 43 mmol, 1 eq.) was added, and the reaction mixture was heated to 120° C. for 4 hrs. LCMS analysis showed that desired mass was detected and reaction was completely. After cooling to R.T., water was added, and the solid precipitate was collected by filtration, washed with water, and air dried to afford 4-chloro-1,2-dimethyl-5-nitro-1H-benzo[d]imidazole (1.25 g, yield: 25.8%) as a light yellow solid.

LC-MS m/z: 226.1 (M+1)⁺.

### Step 4: tert-butyl (1R,4R)-5-(1,2-dimethyl-5-nitro-1H-benzo[d]imidazol-4-yl)-2,5-diazabicyclo[2.2.1]heptane-2-carboxylate

A solution of tert-butyl (1R,4R)-2,5-diazabicyclo[2.2.1]heptane-2-carboxylate (200.0 mg, 1.01 mmol, 1 eq) and 4-chloro-1,2-dimethyl-5-nitro-1H-benzo[d]imidazole (250.6 mg, 1.11 mmol, 1.1 eq) in DMSO (2 mL) was treated with TEA (0.21 mL). Then the reaction was heated to 80° C. for 3 h. LCMS analysis showed that desired mass was detected and reaction was completely. After cooling to room temperature, the solid precipitate was collected by filtration, washed with water, and air dried to afford tert-butyl (1R,4R)-5-(1,2-dimethyl-5-nitro-1H-benzo[d]imidazol-4-yl)-2,5-diazabicyclo[2.2.1]heptane-2-carboxylate (241.3 mg, Yield: 61.66%) as an orange solid.

LC-MS m/z: 388.3 (M+1)⁺.

### Step 5: tert-butyl (1R,4R)-5-(5-amino-1,2-dimethyl-1H-benzo[d]imidazol-4-yl)-2,5-diazabicyclo[2.2.1]heptane-2-carboxylate

A solution of tert-butyl (1R,4R)-5-(1,2-dimethyl-5-nitro-1H-benzo[d]imidazol-4-yl)-2,5-diazabicyclo[2.2.1]heptane-2-carboxylate (241.3 mg, 0.623 mmol, 1 eq) and Pd/C (100.0 mg) in MeOH (5 mL) was stirred at rt under H₂ atmosphere (60 psi) for 16 h. LCMS analysis of the reaction mixture showed full conversion to the desired product. The reaction mixture was filtered and concentrated to afford tert-butyl (1R,4R)-5-(5-amino-1,2-dimethyl-1H-benzo[d]imidazol-4-yl)-2,5-diazabicyclo[2.2.1]heptane-2-carboxylate (239.5 mg, yield: 101.6%) as dark purple solid.

LC-MS m/z: 358.2 (M+1)⁺

### Step 6: tert-butyl (1R,4R)-5-(5-(3-fluoro-2-(2-fluoro-6-methoxyphenyl)isonicotinamido)-1,2-dimethyl-1H-benzo[d]imidazol-4-yl)-2,5-diazabicyclo[2.2.1]heptane-2-carboxylate

A solution of tert-butyl (1R,4R)-5-(5-amino-1,2-dimethyl-1H-benzo[d]imidazol-4-yl)-2,5-diazabicyclo[2.2.1]heptane-2-carboxylate (149.Omg, 0.417 mmol, 1 eq.), 3-fluoro-2-(2-fluoro-6-methoxyphenyl)isonicotinic acid (121.6 mg, 0.459 mmol, 1.1 eq.) and DIPEA (0.15 mL, 0.459 mmol, 2 eq) in DMF (2 mL) was added HATU (237.74 mg, 0.625 mmol, 1.5 eq.). The reaction mixture was stirred at room temperature for 16 hours. LCMS analysis showed that desired mass was detected and reaction was completely. After that the mixture treated with water. The precipitated product was collected by filtration, washed with water and dried by oil pump to afford tert-butyl (1R,4R)-5-(5-(3-fluoro-2-(2-fluoro-6-methoxyphenyl)isonicotinamido)-1,2-dimethyl-1H-benzo[d]imidazol-4-yl)-2,5-diazabicyclo[2.2.1]heptane-2-carboxylate (235.1 mg, Yield: 93.28%) as a green solid.

LC-MS m/z: 605.3 (M+1)⁺.

### Step 7: N-(4-((1S,4S)-2-azabicyclo[2.2.1]heptan-5-yl)-1,2-dimethyl-1H-benzo[d]imidazole-5-yl)-3-fluoro-2-(2-fluoro-6-methoxyphenyl)isonicotinamide

A solution of tert-butyl (1R,4R)-5-(5-(3-fluoro-2-(2-fluoro-6-methoxyphenyl)isonicotinamido)-1,2-dimethyl-1H-benzo[d]imidazol-4-yl)-2,5-diazabicyclo[2.2.1]heptane-2-carboxylate (235.1 mg, 0.389 mmol, 1 eq.) in TFA (2 mL) was stirred for 1 hours at R.T to give a black brown solution. LCMS analysis of the reaction mixture showed full conversion to the desired product. Then the reaction mixture was poured into 20 mL of water and DCM, then the organic layer on phase was separated. The water was basified with NaOH to pH=11, the solid precipitate was collected by filtration, washed with water, and freeze dried to afford Example 7 (62.5 mg, Yield: 50.27%) as a yellow solid.

LC-MS m/z: 504.2 (M+1)⁺.

¹H NMR (400 MHz, CD₃OD): δ 8.66 (d, J=5.0 Hz, 1H), 8.03 (dt, J=5.1, 2.6 Hz, 1H), 8.00 (t, J=5.3 Hz, 1H), 7.55 (td, J=8.5, 6.8 Hz, 1H), 7.19 (d, J=8.7 Hz, 1H), 7.04 (d, J=8.5 Hz, 1H), 6.93 (t, J=8.7 Hz, 1H), 4.21 (s, 1H), 4.12 (t, J=8.3 Hz, 1H), 3.86 (s, 3H), 3.81 (s, 1H), 3.77 (s, 3H), 3.63-3.53 (m, 1H), 3.17 (d, J=8.8 Hz, 1H), 2.79 (d, J=10.3 Hz, 1H), 2.60 (s, 3H), 2.15 (d, J=9.5 Hz, 1H), 1.84 (d, J=9.6 Hz, 1H).

| No. | Structure | Salt form | MS and ¹HNMR |
|---|---|---|---|
| Example 8 | 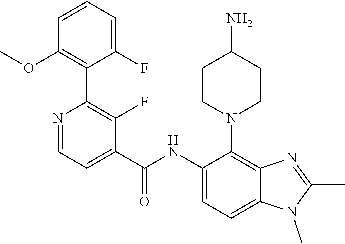 N-(4-(4-aminopiperidin-1-yl)- 1,2-dimethyl-1H- benzo[d]imidazol-5-yl)-3- fluoro-2-(2-fluoro-6- methoxyphenyl)isonicotinamide | HCl Salt | LC-MS m/z: 507.2 (M + 1)⁺ ¹H NMR (400 MHz, CD₃OD) δ 8.67 (d, J = 4.9 Hz, 1H), 8.27 (s, 1H), 7.97 (t, J = 5.2 Hz, 1H), 7.72-7.70 (m, 1H), 7.57-7.48 (m, 1H), 7.02 (d, J = 8.5 Hz, 1H), 6.91 (t, J = 8.8 Hz, 1H), 3.97 (s, 3H), 3.82 (s, 3H), 3.44-3.40 (m, 2H), 3.37-3.33 (m, 2H), 3.32-3,31 (m, 1H), 2.90 (s, 3H), 2.16-2.12 (m, 2H), 1.90-1.86 (m, 2H). |
| Example 9 | 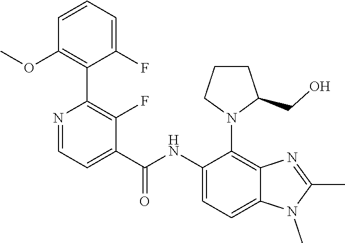 3- fluoro-2-(2-fluoro-6- methoxyphenyl)-N-(4-((S)-2- (hydroxymethyl)pyrrolidin-1- yl)-1,2-dimethyl-1H- benzo[d]imidazol-5- yl)isonicotinamide | HCl Salt | LC-MS m/z: 508.2 (M + 1)⁺ ¹H NMR (400 MHz, CD₃OD) δ 8.66 (d, J = 5.0 Hz, 1H), 8.37 (dd, J = 8.8, 3.9 Hz, 1H), 8.05-8.02 (m, 1H), 7.56 (dd, J = 15.3, 8.4 Hz, 1H), 7.36 (d, J = 8.8 Hz, 1H), 7.05 (d, J = 8.4 Hz, 1H), 6.93 (t, J = 8.4 Hz, 1H), 4.20 (m, 1H), 3.87 (d, J = 8.7 Hz, 3H), 3.81 (s, 3H), 3.46-3.35 (m, 4H), 2.64 (s, 3H), 2.34 (m, 1H), 2.05-1.91 (m, 3H). |
| Example 10 | 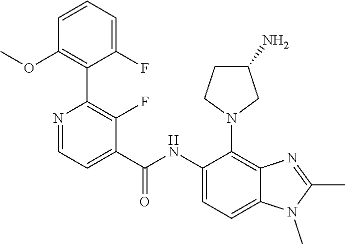 N-(4-((S)-3-aminopyrrolidin-1- yl)-1,2-dimethyl-1H- benzo[d]imidazol-5-yl)-3- fluoro-2-(2-fluoro-6- methoxyphenyl)isonicotinamide | HCOOH Salt | LC-MS m/z: 493.5 (M + 1)⁺ ¹H NMR (400 MHz, CD₃OD) δ 8.63 (d, J = 5.0 Hz, 1H), 8.52 (s, 1H), 8.32- 8.17 (m, 1H), 7.94 (t, J = 5.3 Hz, 1H), 7.55 (td, J = 8.5, 6.7 Hz, 1H), 7.35 (d, J = 8.8 Hz, 1H), 7.09-6.81 (m, 2H), 4.02 (ddq, J = 6.2, 4.1, 1.9 Hz, 1H), 3.85 (s, 3H), 3.79 (s, 3H), 3.61 (tdd, J = 9.4, 6.8, 2.5 Hz, 1H), 3.54-3.40 (m, 2H), 2.65 (s, 3H), 2.52-2.33 (m, 1H), 2.33-2.16 (m, 1H). |

### Example 11: N-(4-((S)-3-aminopyrrolidin-1-yl)-1-(2-methoxyethyl)-2-methyl-1H-benzo[d]imidazol-5-yl)-3-fluoro-2-(2-fluoro-6-methoxyphenyl)isonicotinamide

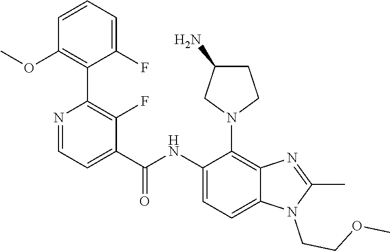

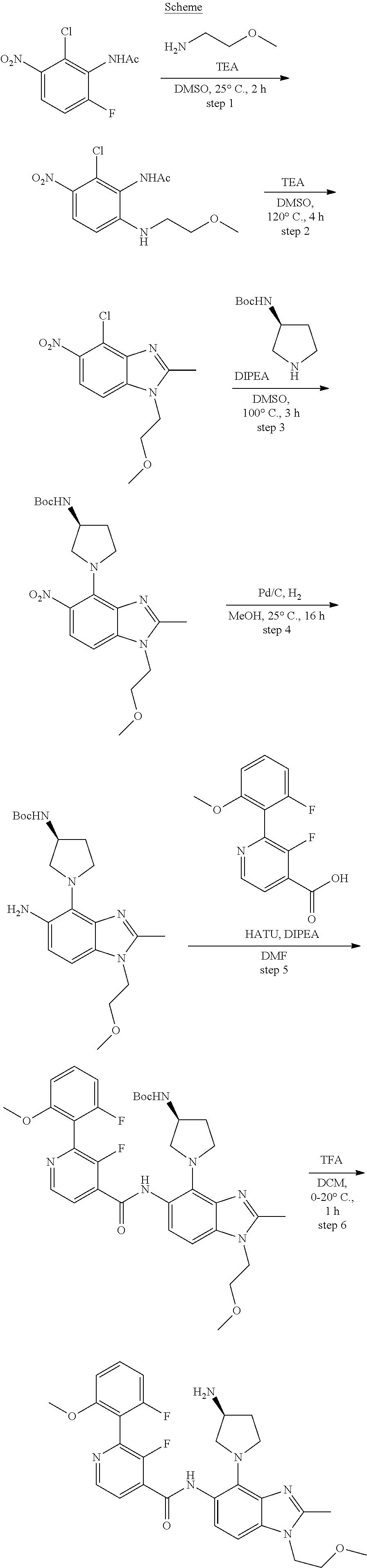

### Step 1: N-(2-chloro-6-((2-methoxyethyl)amino)-3-nitrophenyl)acetamide

A mixture of N-(2-chloro-6-((2-methoxyethyl)amino)-3-nitrophenyl)acetamide (500 mg, 2.15 mmol, 1 eq), methylamine in THF (1.6 mL, 2M) and triethylamine (0.45 mL, 3.22 mmol, 1.5 eq) in DMSO (5 mL) was stirred at 25° C. for 2 hours. After the reaction was completed, the reaction was quenched with water (20 mL) and extracted with EtOAc (20 mL×3). The combined organic phase was washed with brine (20 mL×3), dried over Na2SO4, filtrated and concentrated under reduced pressure to afford N-(2-chloro-6-((2-methoxyethyl)amino)-3-nitrophenyl)acetamide (400 mg, Yield: 38.8%) without further purification as a brown solid.

LC-MS m/z: 288.0 (M+1)⁺.

### Step 2: 4-chloro-1-(2-methoxyethyl)-2-methyl-5-nitro-1H-benzo[d]imidazole

A mixture of N-(2-chloro-6-((2-methoxyethyl)amino)-3-nitrophenyl)acetamide (400 mg, 1.39 mmol, 1 eq) and triethylamine (0.58 mL, 4.17 mmol, 3 eq) in DMSO (5 mL) was stirred at 120° C. for 4 hours. After cooling to room temperature, the reaction was quenched with water (20 mL) and extracted with EtOAc (20 mL×3). The combined organic phase was washed with brine (20 mL×3), dried over Na2SO4, filtrated, and concentrated under reduced pressure to give a residue, which was purified by flash column chromatography (eluting with Petroleum ether/EtOAc=1/1) to afford 4-chloro-1-(2-methoxyethyl)-2-methyl-5-nitro-1H-benzo[d]imidazole (300 mg, Yield: 76.0%) as a grey solid.

LC-MS m/z: 270.1 (M+1)⁺.

### Step 3: tert-butyl (S)-(1-(1-(2-methoxyethyl)-2-methyl-5-nitro-1H-benzo[d]imidazol-4-yl)pyrrolidin-3-yl)carbamate

A mixture of tert-butyl (S)-(1-(1-(2-methoxyethyl)-2-methyl-5-nitro-1H-benzo [d]imidazol-4-yl)pyrrolidin-3-yl)carbamate (100 mg, 0.37 mmol, 1 eq), tert-butyl (S)-pyrrolidin-3-yl-carbamate (104 mg, 0.56 mmol, 1.5 eq) and diisopropylethylamine (0.13 mL, 0.74 mmol, 2 eq) in DMSO was stirred at 100° C. for 3 hours. After cooling to room temperature, the reaction was quenched with water (20 mL) and extracted with EtOAc (20 mL×3). The combined organic phase was washed with brine (20 mL×3), dried over Na2SO4, filtrated and concentrated under reduced pressure to give a residue, which was purified by flash column chromatography (eluting with Petroleum ether/EtOAc (=1/1)) to afford tert-butyl (S)-(1-(1-(2-methoxyethyl)-2-methyl-5-nitro-1H-benzo[d]imidazol-4-yl)pyrrolidin-3-yl) carbamate (145 mg, Yield: 93.2%) as a yellow solid.

LC-MS m/z: 420.2 (M+1)⁺.

### Step 4: tert-butyl (S)-(1-(5-amino-1-(2-methoxyethyl)-2-methyl-1H-benzo[d]imidazol-4-yl)pyrrolidin-3-yl)carbamate

The mixture of Pd/C (72 mg, 10%) and tert-butyl (S)-(1-(5-amino-1-(2-methoxyethyl)-2-methyl-1H-benzo[d]imidazol-4-yl)pyrrolidin-3-yl)carbamate (145 mg, 0.35 mmol) in methanol (3 mL) was hydrogenated under 15 psi for 4 hours. The reaction mixture was filtered and concentrated under reduced pressure to afford tert-butyl (S)-(1-(5-amino-1-(2-methoxyethyl)-2-methyl-1H-benzo[d]imidazol-4-yl)pyrrolidin-3-yl)carbamate (113 mg, Yield: 71.3%) as a light yellow solid.

LC-MS m/z: 390.1 (M+1)⁺.

### Step 5: tert-butyl ((35)-1-(5-(3-fluoro-2-(2-fluoro-6-methoxyphenyl)isonicotinamido)-1-(2-methoxyethyl)-2-methyl-1H-benzo[d]imidazol-4-yl)pyrrolidin-3-yl)carbamate

To a solution of tert-butyl (S)-(1-(5-amino-1-(2-methoxyethyl)-2-methyl-1H-benzo[d]imidazol-4-yl)pyrrolidin-3-yl)carbamate (109 mg, 0.28 mmol, 1.0 eq) and 3-fluoro-2-(2-fluoro-6-methoxyphenyl)isonicotinic acid (74 mg, 0.28 mmol, 1.0 eq) in DMF (3 mL) were added HATU (128 mg, 0.34 mmol, 1.2 eq) and DIPEA (0.24 mL, 1.40 mmol, 5.0 eq). The reaction mixture was stirred at 50° C. for 4 hours. After cooling to room temperature, the reaction was quenched with water (20 mL) and extracted with EtOAc (20 mL×3). The combined organic phase was washed with brine (20 mL×3), dried over anhydrous Na₂SO₄, filtrated and concentrated under reduced pressure to give crude product, which was purified by flash column chromatography (eluting with DCM/MeOH=97/3) to afford tert-butyl ((35)-1-(5-(3-fluoro-2-(2-fluoro-6-methoxyphenyl)isonicotinamido)-1-(2-methoxyethyl)-2-methyl-1H-benzo[d]imidazol-4-yl)pyrrolidin-3-yl)carbamate (112 mg, Yield: 59.7%) as a light yellow solid.

LC-MS m/z: 637.2 (M+1)⁺.

### Step 6: N-(4-((S)-3-aminopyrrolidin-1-yl)-1-(2-methoxyethyl)-2-methyl-1H-benzo[d]imidazol-5-yl)-3-fluoro-2-(2-fluoro-6-methoxyphenyl)isonicotinamide

To a solution tert-butyl ((35)-1-(5-(3-fluoro-2-(2-fluoro-6-methoxyphenyl)isonicotinamido)-1-(2-methoxyethyl)-2-methyl-1H-benzo[d]imidazol-4-yl)pyrrolidin-3-yl)carbamate (97 mg, 0.15 mmol) in DCM (3 mL) was added trifluoroacetic acid (1 mL). The reaction mixture was stirred at 25° C. for 3 hours. The reaction mixture was concentrated to afford Example 11 trifluoroacetate (33.7 mg, Yield: 39.1%) as a yellow solid.

LC-MS m/z: 537.2 (M+1)⁺.

1H NMR (400 MHz, CD₃OD) δ 8.67 (d, J=4.9 Hz, 1H), 8.05 (d, J=8.9 Hz, 1H), 7.94 (t, J=5.1 Hz, 1H), 7.66 (d, J=8.9 Hz, 1H), 7.54 (m, 1H), 7.03 (d, J=8.5 Hz, 1H), 6.92 (m, 1H), 4.62 (t, J=4.5 Hz, 2H), 4.02 (m, 1H), 3.84 (s, 3H), 3.82-3.79 (m, 2H), 3.68 (m, 1H), 3.50-3.44 (m, 2H), 3.33 (s, 1H), 3.32 (s, 3H), 2.91 (s, 3H), 2.50 (m, 1H), 2.17 (m, 1H).

### Example 12: N-(2-((S)-3-aminopyrrolidin-1-yl)-4-(4-cyanopyridin-3-yl)phenyl)-3-fluoro-2-(2-fluoro-6-methoxyphenyl)isonicotinamide

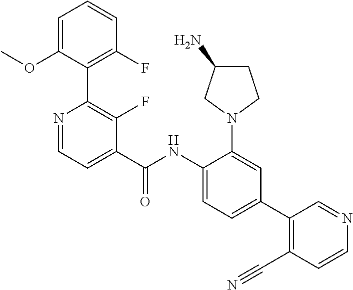 [SMILES: COc1cccc(F)c1-c1nccc(C(=O)Nc2ccc(-c3cnccc3C#N)cc2N2CC[C@H](N)C2)c1F]

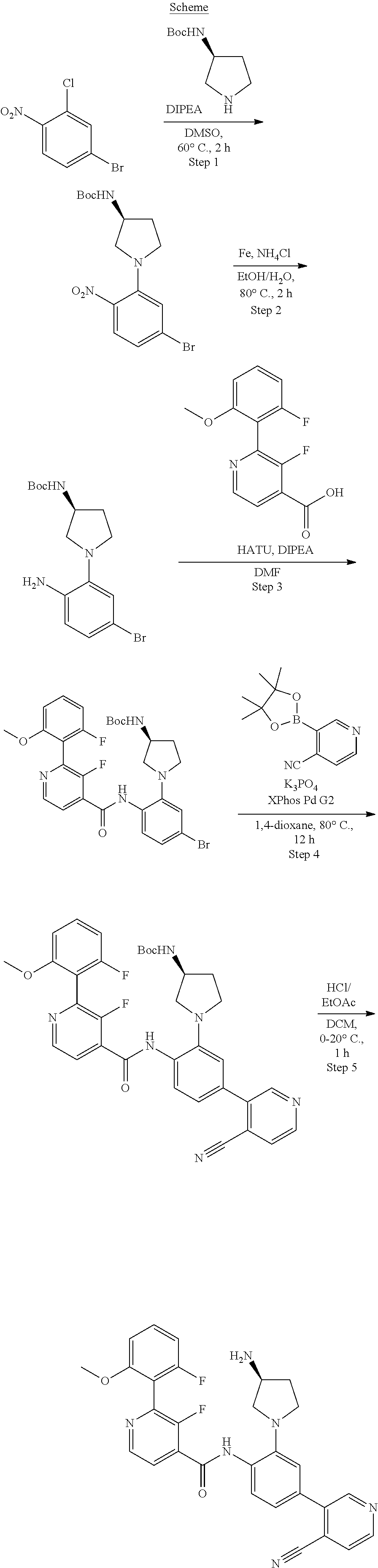

### Step 1: tert-butyl (S)-(1-(5-bromo-2-nitrophenyl)pyrrolidin-3-yl)carbamate

To a solution of 4-bromo-2-fluoro-1-nitrobenzene [500 mg, 2.27 mmol, 1.0 eq] in DMSO were added tert-butyl N-[(35)-pyrrolidin-3-yl]carbamate [508 mg, 2.72 mmol, 1.2 eq] and diisopropylethylamine [587 mg, 4.54 mmol, 2.0 eq]. The reaction mixture was stirred under nitrogen at 60° C. for 2 hrs. Then mixture was diluted with water and extracted with EtOAc (3×30 mL). The organic layer was dried over Na₂SO₄ and evaporated in vacuo to give the crude product. The residue was purified by flash chromatography eluting with Petroleum ether/EtOAc (=10:1) to give tert-butyl (S)-(1-(5-bromo-2-nitrophenyl)pyrrolidin-3-yl)carbamate (660 mg, Yield: 72.3%) as a yellow solid.

LC-MS m/z: 387.2 (M+1)⁺.

### Step 2: tert-butyl (S)-(1-(2-amino-5-bromophenyl)pyrrolidin-3-yl)carbamate

To a mixture of tert-butyl N-[(35)-1-(5-bromo-2-nitrophenyl)pyrrolidin-3-yl]carbamate [300 mg, 0.77 mmol, 1.0 eq] and iron [217 mg, 3.88 mmol, 5.0 eq] in EtOH [8.0 mL] were added ammonium chloride [210 mg, 3.88 mmol, 5.0 eq] in H₂O [8.0 mL] dropwise. The reaction mixture was refluxed for 2 hrs. The mixture was filtered, and the filtrate was collected and concentrated. The residue was purified by flash chromatography eluting with Petroleum ether/EtOAc to give tert-butyl (S)-(1-(2-amino-5-bromophenyl)pyrrolidin-3-yl)carbamate (200 mg, Yield: 70.3%) as a red solid.

LC-MS m/z: 357.2 (M+1)⁺.

### Step 3: tert-butyl((35)-1-(5-bromo-2-(3-fluoro-2-(2-fluoro-6-methoxyphenyl)isonicotinamido) phenyl)pyrrolidin-3-yl)carbamate

To a solution of 3-fluoro-2-(2-fluoro-6-methoxyphenyl)pyridine-4-carboxylic acid [45 mg, 0.16 mmol, 1.0 eq] in DMF were added tert-butyl N-[(35)-1-(2-amino-5-bromophenyl)pyrrolidin-3-yl]carbamate[60 mg, 0.16 mmol, 1.0 eq], 2-(7-azabenzotriazol-1-yl)-N,N,N′,N′-tetramethyluronium hexafluorophosphate [77 mg, 0.20 mmol, 1.3 eq] and diisopropylethylamine [87 mg, 0.67 mmol, 4.2 eq]. The reaction mixture was stirred at 50° C. for 3 hrs. The mixture was diluted with brine solution [5.0 mL] and extracted with EtOAc (3×10 mL). The organic layer was dried over Na₂SO₄ and evaporated in vacuo to give the crude product. The residue was purified via flash chromatography eluting with Petroleum ether/EtOAc=5:1 to give tert-butyl((35)-1-(5-bromo-2-(3-fluoro-2-(2-fluoro-6-methoxyphenyl)isonicotinamido)phenyl)pyrrolidin-3-yl)carbamate (60 mg, Yield: 54.4%).

LC-MS m/z: 604.1 (M+1)⁺.

### Step 4: tert-butyl ((35)-1-(5-(4-cyanopyridin-3-yl)-2-(3-fluoro-2-(2-fluoro-6-methoxyphenyl)isonicotinamido)phenyl)pyrrolidin-3-yl)carbamate

To a solution of tert-butyl N-[(35)-1-{5-bromo-2-[3-fluoro-2-(2-fluoro-6-methoxyphenyl)pyridine-4-amido]phenyl}pyrrolidin-3-yl]carbamate [60 mg, 0.09 mmol, 1.0 eq] in dioxane were added 4-cyanopyidine-3-boric acid ester [46 mg, 0.19 mmol, 2.1 eq], anhydrous potassium phosphate [42.2 mg, 0.19 mmol, 2.1 eq] and XPhos Pd G2 [7.81 mg, 0.01 mmol, 0.11 eq]. The reaction mixture was stirred under nitrogen at 80° C. for 12 hrs. The reaction mixture was concentrated under reduced pressure at 40° C. The residue was purified by flash chromatography eluting with DCM/MeOH=20:1) to give tert-butyl ((35)-1-(5-(4-cyanopyridin-3-yl)-2-(3-fluoro-2-(2-fluoro-6-methoxyphenyl)isonicotinamido)phenyl)pyrrolidin-3-yl)carbamate (50 mg, Yield: 74.5%) as a yellow solid.

LC-MS m/z: 627.2 (M+1)⁺.

### Step 5: N-(2-((S)-3-aminopyrrolidin-1-yl)-4-(4-cyanopyridin-3-yl)phenyl)-3-fluoro-2-(2-fluoro-6-methoxyphenyl)isonicotinamide

To a solution of tert-butyl N-[(35)-1-[5-(4-cyanopyridin-3-yl)-2-[3-fluoro-2-(2-fluoro-6-methoxyphenyl)pyridine-4-amido]phenyl]pyrrolidin-3-yl]carbamate [40 mg, 0.06 mmol] in DCM [1.5 mL] was added a solution of hydrogen chloride-1,4-dioxane [1.5 mL, 4 M]. The reaction mixture was stirred at 20° C. for 1 hour. The reaction mixture was concentrated and washed with DCM. A solid was collected and then freeze-dried to afford Example 12 (25 mg, Yield: 66.4%) as a yellow solid.

LC-MS m/z: 527.2 (M+1)⁺.

¹H NMR (400 MHz, CD₃OD) δ 9.21 (s, 1H), 8.99 (d, J=5.6 Hz, 1H), 8.85 (d, J=5.3 Hz, 1H), 8.33 (d, J=5.6 Hz, 1H), 8.29-8.18 (m, 2H), 7.66 (m, 2H), 7.49 (d, J=8.4 Hz, 1H), 7.12 (d, J=8.5 Hz, 1H), 7.00 (t, J=8.8 Hz, 1H), 4.01 (s, 1H), 3.90 (s, 3H), 3.69 (m, 2H), 3.37 (m, 1H), 3.19 (m, 1H), 2.51 (m, 1H), 2.09 (m, 1H).

| No. | Structure | Salt form | MS and ¹HNMR |
|---|---|---|---|
| Example 13 | 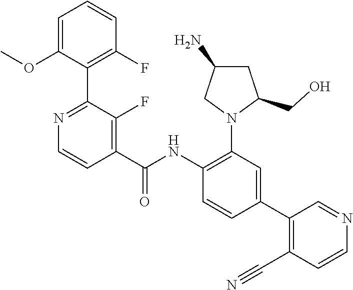 [SMILES: COc1cccc(F)c1-c1nccc(C(=O)Nc2ccc(-c3cnccc3C#N)cc2N2C[C@@H](N)C[C@H]2CO)c1F] | HCOOH Salt | LC-MS m/z: 557.2 (M + 1)⁺ ¹H NMR (400 MHz, CD₃OD) 8.92 (s, 1H), 8.73 (dd, J = 28.4, 5.2 Hz, 3H), 8.00 (dt, J = 10.4, 5.2 Hz, 1H), 7.93 (d, J = 7.7 Hz, 1H), 7.86 (dd, J = 5.1, 0.9 Hz, 1H), 7.59 (dd, J = 20.0, 8.0 Hz, 2H), 7.06 (t, J = 7.3 Hz, 1H), 6.95 (q, J = 10.4 Hz, 1H), 3.94 (s, 1H), 3.86 (s, 1H), 3.81 (s, 2H), 3.76- 3.33 (m, 5H), 2.84-2.72 (m, 1H), 2.03 (d, J = 14.3 Hz, 1H). |
| | N-(2-((2S,4S)-4-amino-2- | | |
| | (hydroxymethyl)pyrrolidin-1-yl)-4- | | |
| | (4-cyanopyridin-3-yl)phenyl)-3- | | |
| | fluoro-2-(2-fluoro-6- | | |
| | methoxyphenyl)isonicotinamide | | |

### Example 14: N-{4-[(3S)-3-aminopyrrolidin-1-yl]-2-(3-fluoropyridin-4-yl)-1-methyl-1,3-benzodiazol-5-yl}-3-fluoro-2-(2-fluoro-6-methoxyphenyl)pyridine-4-carboxamide

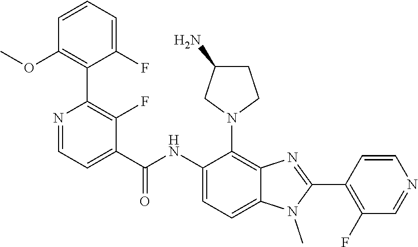 [SMILES: COc1cccc(F)c1-c1nccc(C(=O)Nc2ccc3c(nc(-c4ccncc4F)n3C)c2N2CC[C@H](N)C2)c1F]

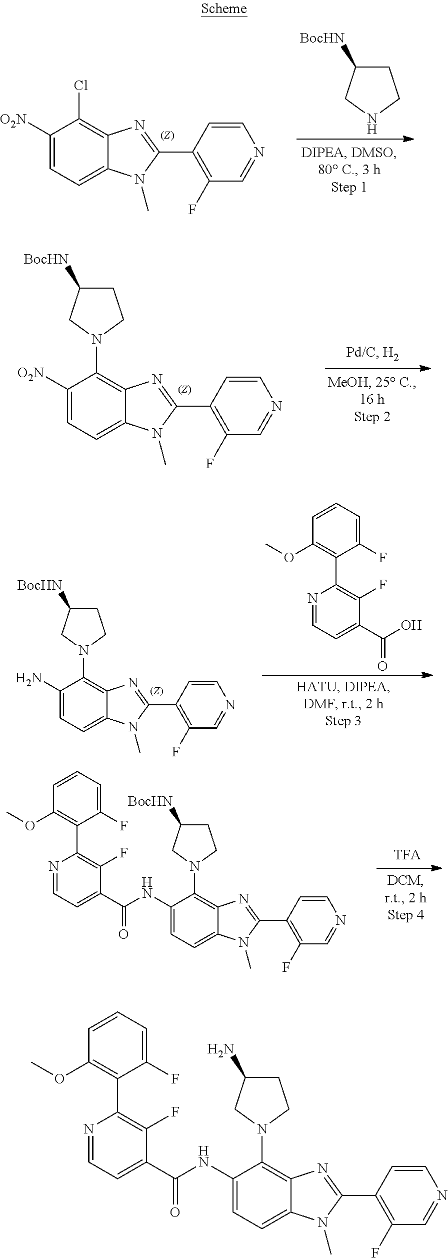

### Step 1: tert-butylN-[(35)-1-[2-(3-fluoropyridin-4-yl)-1-methyl-5-nitro-1,3-benzodiazol-4-yl]pyrrolidin-3-yl]carbamate

To a solution of 4-chloro-2-(3-fluoropyridin-4-yl)-1-methyl-5-nitro-1,3-benzodiazole (100 mg, 0.39 mmol, 1.0 eq) and tert-butyl N-[(35)-pyrrolidin-3-yl]carbamate (73 mg, 0.39 mmol, 1 eq) in DMSO (1.5 mL) was added DIPEA (0.16 mL, 0.78 mmol, 2 eq) at room temperature. The reaction mixture was stirred at 100° C. for 4 hours. After cooling to room temperature, the reaction was quenched with and extracted with EtOAc (10 mL×3). The combined organic phase was washed with brine (10 mL×3), dried over Na2SO4, filtrated and concentrated under reduced pressure to give crude product, which was purified by SGC (UV254, Petroleum ether:EtOAc=1:1) to afford tert-butylN-[(35)-1-[2-(3-fluoropyridin-4-yl)-1-methyl-5-nitro-1,3-benzodiazol-4-yl]pyrrolidin-3-yl]carbamate (80 mg, Yield: 46.2%) as a yellow solid.

LC-MS m/z: 457.2 (M+1)⁺.

### Step 2: tert-butyl N-[(35)-1-[5-amino-2-(3-fluoropyridin-4-yl)-1-methyl-1,3-benzodiazol-4-yl]pyrrolidin-3-yl]carbamate

The mixture of Pd/C (32 mg, 0.29 mmol) and tert-butyl N-[(35)-1-[2-(3-fluoropyridin-4-yl)-1-methyl-5-nitro-1,3-benzodiazol-4-yl]pyrrolidin-3-yl]carbamate (80 mg, 0.15 mmol) in methanol (6 mL) was hydrogenated under 15 psi for 2 hours. The reaction mixture was filtered and concentrated under reduced pressure to afford the tert-butyl N-[(35)-1-[5-amino-2-(3-fluoropyridin-4-yl)-1-methyl-1,3-benzodiazol-4-yl]pyrrolidin-3-yl]carbamate (90 mg, Yield: 96.3%) as a yellow solid.

LC-MS m/z: 427.1 (M+1)⁺.

### Step 3: N-{4-[(3S)-3-aminopyrrolidin-1-yl]-2-(3-fluoropyridin-4-yl)-1-methyl-1,3-benzodiazol-5-yl}-3-fluoro-2-(2-fluoro-6-methoxyphenyl)pyridine-4-carboxamide

To a solution of tert-butyl (S)-(1-(5-amino-2-(3-fluoropyridin-4-yl)-1-methyl-1H-benzo[d]imidazol-4-yl)pyrrolidin-3-yl)carbamate (80 mg, 0.18 mmol, 1.0 eq) and 3-fluoro-2-(2-fluoro-6-methoxyphenyl)isonicotinic acid (49.75 mg, 0.18 mmol, 1. eq) in DMF (15 mL) was added HATU (85.6 mg, 0.22 mmol, 1.2 eq) and DIPEA (0.12 mL, 0.75 mmol, 4.0 eq) at room temperature. The reaction temperature was increased to 50° C., and the reaction was stirred at 50° C. for 4 hours. After cooling to room temperature, the reaction was quenched with water (5 mL) and extracted with EtOAc (5 mL×3). The combined organic phase was washed with brine (10 mL×3), dried over anhydrous Na₂SO₄, filtrated and concentrated under reduced pressure to give crude product, which was purified by flash column chromatography (eluting with DCM/MeOH=40/1) to afford N-{4-[(3S)-3-aminopyrrolidin-1-yl]-2-(3-fluoropyridin-4-yl)-1-methyl-1,3-benzodiazol-5-yl}-3-fluoro-2-(2-fluoro-6-methoxyphenyl)pyridine-4-carboxamide (35 mg, Yield: 26.3%) as a yellow solid.

LC-MS m/z: 674.3 (M+1)⁺.

### Step 4: (S)—N-(4-(3-aminopyrrolidin-1-yl)-2-(methoxymethyl)-1-methyl-1H-benzo[d]imidazol-5-yl)-2-(2,6-difluorophenyl)-3-oxo-2,3-dihydropyridazine-4-carboxamide

To a solution N-{4-[(3S)-3-aminopyrrolidin-1-yl]-2-(3-fluoropyridin-4-yl)-1-methyl-1,3-benzodiazol-5-yl}-3-fluoro-2-(2-fluoro-6-methoxyphenyl)pyridine-4-carboxamide (35 mg, 0.052 mmol, 1.0 eq) in DCM (0.5 mL) was added a solution of trifluoroacetic acid in DCM (0.3 mL, 4M, 30.0 eq) at 25° C. The reaction mixture was continued to stir at 25° C. for 2 hours. The reaction mixture was concentrated and washed with MTBE to afford Example 14 (8.7 mg, Yield: 23.3%) as a yellow solid.

LC-MS m/z: 574.2 (M+1)⁺.

¹H NMR (400 MHz, CD₃OD) δ 8.70 (d, J=1.6 Hz, 1H), 8.58 (d, J=4.8 Hz, 2H), 8.22 (d, J=8.3 Hz, 1H), 7.89 (s, 1H), 7.77-7.65 (m, 1H), 7.45 (dd, J=15.3, 8.5 Hz, 2H), 6.94 (d, J=8.4 Hz, 1H), 6.82 (t, J=8.7 Hz, 1H), 3.90 (d, J=16.2 Hz, 1H), 3.85 (dd, J=12.2, 6.4 Hz, 1H), 3.80 (d, J=1.1 Hz, 3H), 3.74 (s, 3H), 3.61 (dd, J=17.1, 8.6 Hz, 1H), 3.52 (d, J=11.9 Hz, 1H), 3.47-3.38 (m, 1H), 2.34 (dt, J=15.5, 7.6 Hz, 1H), 2.11 (s, 1H).

### Example 15: N-{4-[(3S)-3-aminopyrrolidin-1-yl]-2-(methoxymethyl)-1-methyl-1,3-benzodiazol-5-yl}-3-fluoro-2-(2-fluoro-6-methoxyphenyl) pyridine-4-carboxamide

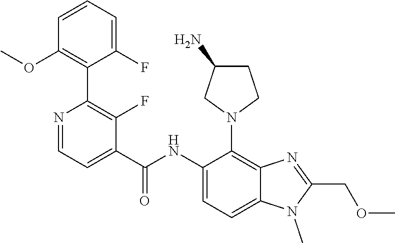

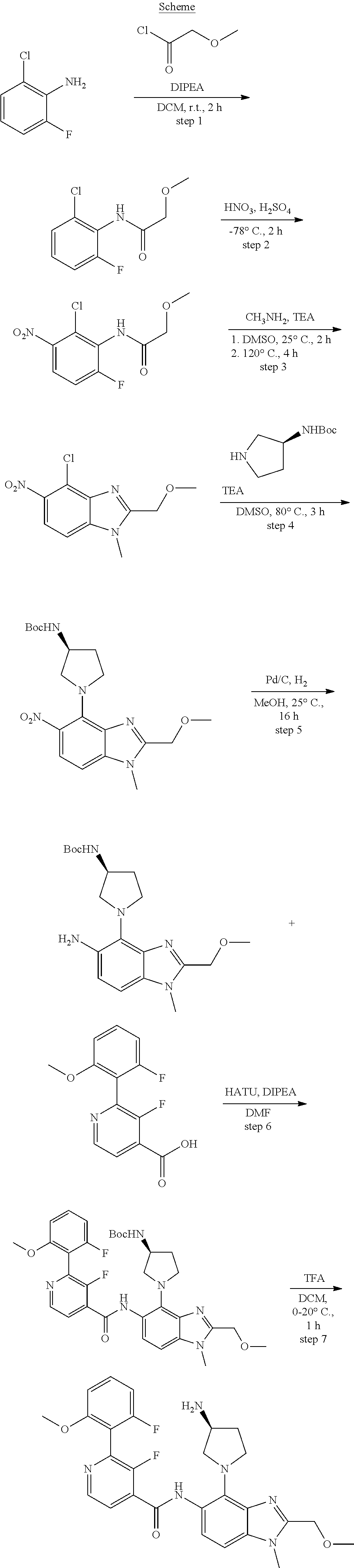

### Step 1: N-(2-chloro-6-fluorophenyl)-2-methoxyacetamide

To a solution of 2-chloro-6-fluoroaniline (2.0 g, 13.7 mmol, 1.0 eq) and DIPEA (2.66 g, 20.6 mmol, 1.5 eq) in DCM (20 mL) stirred at 0° C. was added a solution of 2-methoxyacetyl chloride (1.75 g, 16.4 mmol, 1.2 eq) in DCM (10 ml) dropwise. The mixture was stirred at 20° C. for 2 h. After completion, the reaction mixture was concentrated under reduced pressure at 35° C. The residue was purified by flash chromatography and eluted with Petroleum ether/EtOAc=5:1 to give N-(2-chloro-6-fluorophenyl)-2-methoxyacetamide (1.8 g, Yield: 57.7%) as a white solid.

### Step 2: N-(2-chloro-6-fluoro-3-nitrophenyl)-2-methoxyacetamide

To a solution of N-(2-chloro-6-fluorophenyl)-2-methoxyacetamide (1.8 g, 8.3 mmol, 1.0 eq) in H₂SO₄ (10 mL) stirred at −55° C. was added a solution of concentrated nitric acid (0.6 mL, 11.6 mmol, 1.4 eq) in H₂SO₄ (2 mL) dropwise. The reaction mixture was stirred at −55° C. for 2 h. The resulting mixture was warmed to room temperature and poured into ice-water (30 mL) and the resulting precipitate filtered. The filter cake was dissolved in DCM (80 mL), dried over Na₂SO₄ and concentrated to give N-(2-chloro-6-fluoro-3-nitrophenyl)-2-methoxyacetamide (1.2 g, yield: 49.4%).

LC-MS m/z: 263.1 (M+1)⁺.

### Step 2: N-(2-chloro-6-fluoro-3-nitrophenyl)-2,2,2-trifluoroacetamide

A solution of compound N-(2-chloro-6-fluorophenyl)-2,2,2-trifluoroacetamide (2.82 g, 11.7 mmol, 1.0 eq) in sulfuric acid (25 mL) was cooled to 0° C. and potassium nitrate (1.3 g, 12.8 mmol, 1.1 eq) was added in portions. The resulting mixture was stirred at 0° C. for 20 minutes. After completion, the mixture was poured into ice-water (30 mL) and the resulting precipitate was filtered. The filter cake was dissolved in DCM (80 mL), dried over Na₂SO₄ and concentrated to give the compound N-(2-chloro-6-fluoro-3-nitrophenyl)-2,2,2-trifluoroacetamide (2.94 g, Yield: 78.6%).

¹H NMR (400 MHz, d₆-DMSO) δ 11.91 (s, 1H), 8.30 (dd, J=8.8, 5.2 Hz, 1H), 7.76 (t, J=8.8 Hz, 1H).

### Step 3: 4-chloro-1-methyl-5-nitro-2-(trifluoromethyl)-1H-benzo[d]imidazole

A mixture of compound N-(2-chloro-6-fluoro-3-nitrophenyl)-2,2,2-trifluoroacetamide (2.94 g, 10.3 mmol, 1.0 eq.), a solution of CH₃NH₂ (320 mg, 10.3 mmol, 33%, 1.0 eq.) in THF (5.15 ml, 2M), and TEA (1.43 mL, 10.3 mmol, 1.0 eq) in DMSO (2 mL) was stirred at 25° C. for 2 hours. The mixture was then sparged with N₂ for 30 mins, additional TEA (1.43 mL, 10.3 mmol, 1 eq) was added, and the reaction mixture was heated to 120° C. and stirred at the same temperature for 4 hours. After cooling to room temperature, the mixture was diluted with water, and extracted with EtOAc (50 mL×2). The organic phase was washed with brine (50 mL×3), dried over Na₂SO₄, filtrated and concentrated in vacuo to give a residue which was purified by SGC (UV254, Petroleum ether:EtOAc=10:1) to afford 4-chloro-1-methyl-5-nitro-2-(trifluoromethyl)-1H-benzo[d]imidazole (490 mg, Yield: 16.5%) as a yellow solid.

¹H NMR (400 MHz, d₆-DMSO): δ 8.22 (d, J=9.2 Hz, 1H), 8.05 (d, J=9.2 Hz, 1H), 4.06 (d, J=0.2 Hz, 3H).

### Step 4: tert-butyl (S)-(1-(1,5-dimethyl-2-(trifluoromethyl)-1H-benzo[d]imidazole-4-yl) pyrrolidin-3-yl) carbamate

To a solution of compound 4-chloro-1-methyl-5-nitro-2-(trifluoromethyl)-1H-benzo[d]imidazole (300 mg, 1.08 mmol, 1.0 eq) and compound tert-butyl (S)-pyrrolidin-3-ylcarbamate (300 mg, 1.61 mmol, 1.5 eq) in DMSO (5 mL) was added DIPEA (555 mg, 4.30 mmol, 4 eq). The mixture was stirred at 100° C. for 1 hour. After cooling to room temperature, the mixture was diluted with water, and extracted with EtOAc (10 mL×2). The organic phase was washed with brine (10 mL×3), dried over Na₂SO₄, filtrated, and concentrated in vacuo to give a residue which was purified by SGC (UV254, Petroleum ether:EtOAc=10:1) to afford tert-butyl (S)-(1-(1,5-dimethyl-2-(trifluoromethyl)-1H-benzo[d]imidazole-4-yl)pyrrolidine-3-yl)carbamate (450 mg, Yield: 97.6%) as a yellow solid.

¹H NMR (400 MHz, d₆-DMSO) δ 7.86 (d, J=9.2 Hz, 1H), 7.25 (d, J=4.8 Hz, 1H), 7.05 (d, J=9.2 Hz, 1H), 4.10 (d, J=3.6 Hz, 1H), 3.94 (s, 4H), 3.82-3.64 (m, 2H), 3.32-3.27 (s, 1H), 2.15-2.07 (m, 1H), 1.94 (m, 1H), 1.37 (s, 9H).

### Step 5: tert-butyl (S)-(1-(5-amino-1-methyl-2-(trifluoromethyl)-1H-benzo[d]imidazol-4-yl)pyrrolidin-3-yl)carbamate

10% Pd/C (50 mg, 0.47 mmol) was added to the solution of compound tert-butyl (S)-(1-(1,5-dimethyl-2-(trifluoromethyl)-1H-benzo[d]imidazole-4-yl)pyrrolidin-3-yl)carbamate (450 mg, 1.06 mmol) in methanol (8 mL), and the reaction mixture was hydrogenated under 15 psi for 2 hours. The reaction mixture was filtered and concentrated to afford the compound tert-butyl (5)-(1-(5-amino-1-methyl-2-(trifluoromethyl)-1H-benzo[d]imidazol-4-yl)pyrrolidin-3-yl)carbamate (300 mg, Yield: 71.7%) as a dark purple solid.

¹H NMR (400 MHz, d₆-DMSO) δ 7.48 (d, J=7.6 Hz, 1H), 7.23 (d, J=8.4 Hz, 1H), 6.91 (d, J=8.4 Hz, 1H), 4.88 (s, 2H), 4.15 (s, 1H), 3.85 (s, 3H), 3.61-3.54 (m, 1H), 3.30-3.22 (m, 2H), 3.13-3.10 (m, 1H), 2.28-2.09 (m, 1H), 1.86-1.71 (m, 1H), 1.39 (s, 9H).

### Step 6: tert-butyl (S)-(1-(5-(2-(2,6-difluorophenyl)-3-oxo-2,3-dihydropyridazine-4-carboxamido)-1-methyl-2-(trifluoromethyl)-1H-benzo[d]imidazol-4-yl)pyrrolidin-3-yl)carbamate

To a solution of tert-butyl (S)-(1-(5-amino-1-methyl-2-(trifluoromethyl)-1H-benzo[d]imidazol-4-yl) pyrrolidin-3-yl)carbamate (140 mg, 0.35 mmol, 1.0 eq) and 2-(2,6-difluorophenyl)-3-oxo-2,3-dihydropyridazine-4-carboxylic acid (88 mg, 0.35 mmol, 1.0 eq) in DMF (2 mL) were added HATU (200 mg, 0.53 mmol, 1.5 eq) and DIPEA (0.23 mL, 1.4 mmol, 4.0 eq). The mixture was stirred at 50° C. for 16 hours. After cooling to the room temperature, the mixture was diluted with water and extracted with EtOAc (5 mL×3). The organic phase was washed with brine (10 mL×3), dried over Na₂SO₄, filtrated and concentrated in vacuo to give a residue which was purified by SGC (UV254, Petroleum ether:EtOAc=10:1) to afford tert-butyl(S)-(1-(5-(2-(2,6-difluorophenyl)-3-oxo-2,3-dihydropyridazine-4-carboxamido)-1-methyl-2-(trifluoromethyl)-1H-benzo[d]imidazol-4-yl)pyrrolidin-3-yl)carbamate (180 mg, Yield: 92.4%) as a yellow solid.

¹H NMR (400 MHz, d₆-DMSO) δ 11.62 (s, 1H), 8.48 (d, J=4.4 Hz, 1H), 8.38 (d, J=4.4 Hz, 1H), 8.35 (s, 1H), 7.79-7.63 (m, 1H), 7.53-7.33 (m, 3H), 7.01 (d, J=6.8 Hz, 1H), 4.10 (s, 1H), 3.95 (s, 3H), 3.78-3.70 (m, 1H), 3.49-3.40 (m, 1H), 3.34 (s, 1H), 2.69 (s, 1H), 2.16-2.04 (m, 1H), 1.81-1.71 (m, 1H), 1.34 (s, 9H).

### Step7: (S)—N-(4-(3-aminopyrrolidin-1-yl)-1-methyl-2-(trifluoromethyl)-1H-benzo[d]imidazol-5-yl)-2-(2,6-difluorophenyl)-3-oxo-2,3-dihydropyridazine-4-carboxamide

To a solution of tert-butyl (S)-(1-(5-amino-1-methyl-2-(trifluoromethyl)-1H-benzo[d]imidazol-4-yl) pyrrolidin-3-yl)carbamate (80 mg, 0.13 mmol, 1.0 eq) in DCM (1.5 mL) was added a solution of HCl in 1,4-dioxane (1 mL, 4M, 30.0 eq). The mixture was stirred at 25° C. for 2 hours. The reaction mixture was concentrated to afford the Example 39 (57 mg, Yield: 85.6%) as a yellow solid.

LC-MS m/z: 534.1 (M+1)⁺.

1HNMR (400 MHz, d₆-DMSO) δ 11.59 (s, 1H), 8.51 (d, J=4.0 Hz, 1H), 8.46 (d, J=9.2 Hz, 1H), 8.40 (d, J=4.0 Hz, 1H), 7.98 (s, 2H), 7.80-7.69 (m, 1H), 7.58 (d, J=8.8 Hz, 1H), 7.45 (t, J=7.6 Hz, 2H), 3.97 (s, 3H), 3.86 (d, J=6.8 Hz, 2H), 3.59-3.46 (m, 2H), 3.46-3.37 (m, 1H), 2.36-2.23 (m, 1H), 1.94-1.83 (m, 1H).

| | | Salt | |
|---|---|---|---|
| No. | Structure | form | MS and ¹HNMR |
| Example 40 | 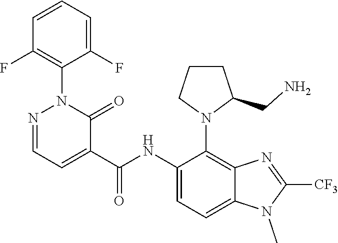 [SMILES: Cn1c(C(F)(F)F)nc2c(N3CCC[C@H]3CN)c(NC(=O)c3ccnn(-c4c(F)cccc4F)c3=O)ccc21] (S)-N-(4-(2- (aminomethyl)pyrrolidin-1-yl)-1- methyl-2-(trifluoromethyl)-1H- benzo[d]imidazol-5-yl)-2-(2,6- difluorophenyl)-3-oxo-2,3- dihydropyridazine-4-carboxamide | HCl Salt | LC-MS m/z: 548.6 (M + 1)⁺ ¹H NMR (400 MHz, d₆-DMSO) δ 11.88 (s, 1H), 8.54 (d, J = 9.0 Hz, 1H), 8.49 (d, J = 4.2 Hz, 1H), 8.39 (d, J = 4.2 Hz, 1H), 7.81 (s, 2H), 7.78-7.70 (m, 1H), 7.66 (d, J = 9.0 Hz, 1H), 7.49-7.42 (m, 1H), 4.56-4.48 (m, 1H), 3.98 (s, 3H), 3.40-3.36 (m, 1H), 3.07-3.03 (m, 1H), 2.62 (m, 2H), 2.31- 2.18 (m, 1H), 2.00-1.80 (m, 3H). |
| Example 41 | 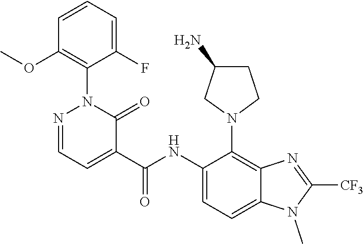 [SMILES: COc1cccc(F)c1-n1nccc(C(=O)Nc2ccc3c(nc(C(F)(F)F)n3C)c2N2CC[C@H](N)C2)c1=O] (S)-N-(4-(3-aminopyrrolidin-1-yl)- 1-methyl-2-(trifluoromethyl)-1H- benzo[d]imidazol-5-yl)-2-(2- fluoro-6-methoxyphenyl)-3-oxo- 2,3-dihydropyridazine-4- carboxamide | HCl Salt | LC-MS m/z: 546.1 (M + 1)+ ¹H NMR (400 MHz, d₆-DMSO) δ 11.73 (s, 1H), 8.46 (d, J = 4.4 Hz, 1H), 8.44 (d, J = 3.2 Hz, 1H), 8.39 (d, J = 4.4 Hz, 1H), 7.98 (s, 2H), 7.70-7.49 (m, 2H), 7.16-7.10 (m, 2H), 3.96 (s, 3H), 3.88-3.82 (m, 5H), 3.58-3.44 (m, 3H), 2.32-2.24 (m, 1H), 1.93-1.81 (m, 1H). |

### Example 42: N-{4-[(35)-3-aminopyrrolidin-1-yl]-2-(3-fluoropyridin-4-yl)-1-methyl-1,3-benzodiazol-5-yl}-2-(2,6-difluorophenyl)-3-oxopyridazine-4-carboxamide

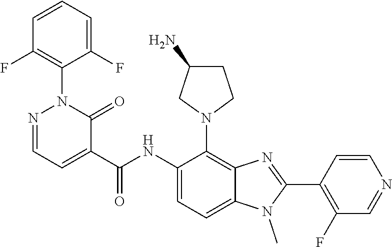 [SMILES: Cn1c(-c2ccncc2F)nc2c(N3CC[C@H](N)C3)c(NC(=O)c3ccnn(-c4c(F)cccc4F)c3=O)ccc21]

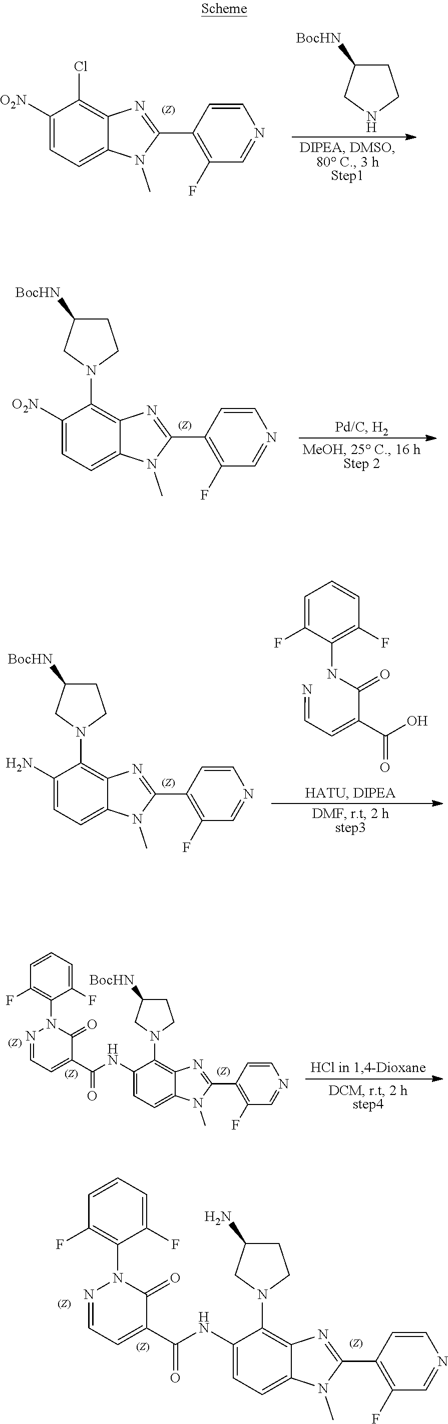

### Step 1: tert-butyl N-[(35)-1-[2-(3-fluoropyridin-4-yl)-1-methyl-5-nitro-1,3-benzodiazol-4-yl]pyrrolidin-3-yl]carbamate

To a solution of 4-chloro-2-(3-fluoropyridin-4-yl)-1-methyl-5-nitro-1,3-benzodiazole (100 mg, 0.39 mmol, 1.0 eq) and tert-butyl N-[(35)-pyrrolidin-3-yl]carbamate (73 mg, 0.39 mmol, 1 eq) in DMSO (1.5 mL) was added DIPEA (0.16 mL, 0.78 mmol, 2 eq) at room temperature. The reaction mixture was stirred at 100° C. for 4 hours. After cooling to room temperature, the reaction was quenched with and extracted with EtOAc (10 mL×3). The combined organic phase was washed with brine (10 mL×3), dried over Na₂SO₄, filtrated and concentrated under reduced pressure to give crude product, which was purified by SGC (UV254, Petroleum ether:EtOAc=1:1) to afford tert-butyl N-[(35)-1-[2-(3-fluoropyridin-4-yl)-1-methyl-5-nitro-1,3-benzodiazol-4-yl]pyrrolidin-3-yl]carbamate (80 mg, Yield: 46.2%) as a yellow solid.

LC-MS m/z: 457.2 (M+1)⁺.

### Step 2: tert-butyl N-[(35)-1-[5-amino-2-(3-fluoropyridin-4-yl)-1-methyl-1,3-benzodiazol-4-yl]pyrrolidin-3-yl]carbamate

The mixture of Pd/C (32 mg, 0.29 mmol) and tert-butyl N-[(35)-1-[2-(3-fluoropyridin-4-yl)-1-methyl-5-nitro-1,3-benzodiazol-4-yl]pyrrolidin-3-yl]carbamate (80 mg, 0.15 mmol) in methanol (6 mL) was hydrogenated under 15 psi for 2 hours. The reaction mixture was filtered and concentrated under reduced pressure to afford the tert-butyl N-[(35)-1-[5-amino-2-(3-fluoropyridin-4-yl)-1-methyl-1,3-benzodiazol-4-yl]pyrrolidin-3-yl]carbamate (90 mg, Yield: 96.3%) as a yellow solid.

LC-MS m/z: 427.1 (M+1)⁺.

### Step 3: tert-butyl N-[(35)-1-{5-[2-(2,6-difluorophenyl)-3-oxopyridazine-4-amido]-2-(3-fluoropyridin-4-yl)-1-methyl-1,3-benzodiazol-4-yl}pyrrolidin-3-yl]carbamate

To a mixture of tert-butyl N-[(35)-1-[5-amino-2-(3-fluoropyridin-4-yl)-1-methyl-1,3-benzodiazol-4-yl]pyrrolidin-3-yl]carbamate (50 mg, 0.12 mmol, 1.0 eq) and 2-(2,6-difluorophenyl)-3-oxopyridazine-4-carboxylic acid (30 mg, 0.12 mmol, 1.0 eq) in DMF (2 mL) were added HATU (54 mg, 0.14 mmol, 1.2 eq) and DIPEA (0.4 mL, 0.48 mmol, 4.0 eq). The reaction mixture was stirred at 50° C. for 2 hours. After cooling to room temperature, the reaction was quenched with water (50 mL) and extracted with EtOAc (10 mL×3). The combined organic phase was washed with brine (50 mL), dried over Na₂SO₄, filtrated and concentrated under reduced pressure to give crude product, which was purified by SGC (UV254, Petroleum ether:EtOAc=3:1) to afford tert-butyl N-[(35)-1-{5-[2-(2,6-difluorophenyl)-3-oxo-pyridazine-4-amido]-2-(3-fluoropyridin-4-yl)-1-methyl-1,3-benzodiazol-4-yl}pyrrolidin-3-yl]carbamate (40 mg, Yield: 49.1%) as a yellow solid.

¹H NMR (400 MHz, CD₃OD) δ 8.64 (s, 1H), 8.52-8.50 (m, 2H), 8.32 (d, J=4.2 Hz, 1H), 8.25 (d, J=4.2 Hz, 1H), 7.73 (d, J=5.1 Hz, 1H), 7.61-7.47 (m, 1H), 7.34 (d, J=8.8 Hz, 1H), 7.17 (t, J=8.6 Hz, 2H), 4.12 (s, 1H), 3.75-3.73 (m, 4H), 3.32-3.31 (m, 2H), 2.21-2.04 (m, 1H), 1.82-1.79 (m, 1H), 1.43-1.26 (m, 2H), 1.19 (s, 9H).

### Step 3: N-{4-[(35)-3-aminopyrrolidin-1-yl]-2-(3-fluoropyridin-4-yl)-1-methyl-1,3-benzodiazol-5-yl}-2-(2,6-difluorophenyl)-3-oxopyridazine-4-carboxamide

To a solution of tert-butyl N-[(35)-1-{5-[2-(2,6-difluorophenyl)-3-oxopyridazine-4-amido]-2-(3-fluoropyridin-4-yl)-1-methyl-1,3-benzodiazol-4-yl}pyrrolidin-3-yl]carbamate (30 mg, 0.045 mmol, 1.0 eq) in DCM (3 mL) was added HCl in 1,4-dioxane (0.5 mL). The reaction mixture was stirred at 25° C. for 2 hours. The reaction mixture was concentrated and washed with tert-butyl methyl ether to afford Example 42 (14 mg, Yield: 49.6%) as a yellow solid. LC-MS m/z: 561.2 (M+1)⁺.

¹H NMR (400 MHz, CD₃OD) δ 8.66 (s, 1H), 8.60 (d, J=9.0 Hz, 1H), 8.55 (d, J=4.7 Hz, 1H), 8.35-8.34 (m, 1H), 8.32-8.31 (m, 1H), 7.70 (s, 1H), 7.65-7.52 (m, 1H), 7.44 (d, J=9.0 Hz, 1H), 7.20-7.17 (m, 2H), 3.99-3.90 (m, 1H), 3.80-3.79 (m, 1H), 3.74 (s, 3H), 3.43-3.39 (m, 2H), 3.25-3.24 (m, 2H), 2.42-2.40 (m, 1H), 1.90-1.88 (m, 1H).

| No. | Structure | Salt form | MS and ¹HNMR |
|---|---|---|---|
| Example 43 | 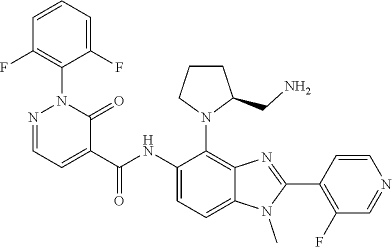 [SMILES: Cn1c(-c2ccncc2F)nc2c(N3CCC[C@H]3CN)c(NC(=O)c3ccnn(-c4c(F)cccc4F)c3=O)ccc21] N-(4-(2-(aminomethyl)pyrrolidin- 1-yl)-2-(3-fluoropyridin-4-yl)-1- methyl-1H-benzo[d]imidazol-5-yl)- 2-(2,6-difluorophenyl)-3-oxo-2,3- dihydropyridazine-4-carboxamide | HCl Salt | LC-MS m/z: 574.5 (M + 1)⁺. ¹HNMR (400 MHz, CD₃OD) δ 8.70 (s, 1H), 8.57 (d, J = 5.0 Hz, 1H), 8.50 (d, J = 9.0 Hz, 1H), 8.38 (d, J = 4.2 Hz, 1H), 8.32 (d, J = 4.2 Hz, 1H), 7.76 (t, J = 5.4 Hz, 1H), 7.60- 7.52 (m, 1H), 7.46 (d, J = 9.0 Hz, 1H), 7.22-7.15 (m, 2H), 3.76 (s, 3H), 3.37-3.35 (m, 1H), 3.14-3.08 (m, 1H), 2.80 (d, J = 4.6 Hz, 2H), 2.41-2.29 (m, 1H), 2.04-1.85 (m, 3H), 1.81-1.71 (m, 1H). |

### Example 44: (S)—N-(4-(3-aminopyrrolidin-1-yl)-2-(methoxymethyl)-1-methyl-1H-benzo[d]imidazol-5-yl)-2-(2,6-difluorophenyl)-3-oxo-2,3-dihydropyridazine-4-carboxamide

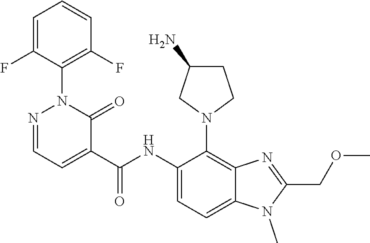 [SMILES: COCc1nc2c(N3CC[C@H](N)C3)c(NC(=O)c3ccnn(-c4c(F)cccc4F)c3=O)ccc2n1C]

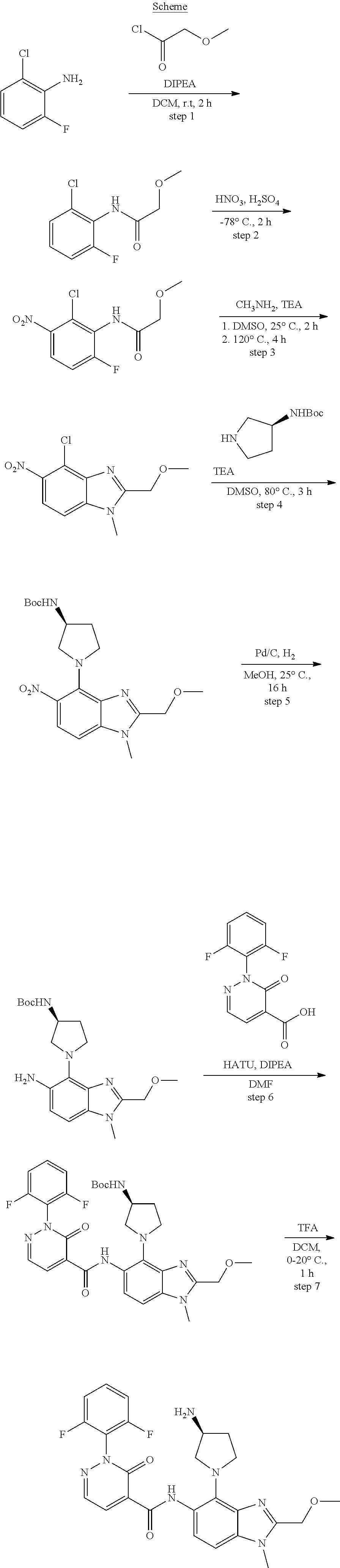

### Step 1: N-(2-chloro-6-fluorophenyl)-2-methoxyacetamide

To a solution of 2-chloro-6-fluoroaniline (2.0 g, 13.7 mmol, 1.0 eq) and DIPEA (2.66 g, 20.6 mmol, 1.5 eq) in DCM (20 mL) stirred at 0° C. was added a solution of 2-methoxyacetyl chloride (1.75 g, 16.4 mmol, 1.2 eq) in DCM (10 ml) dropwise. The mixture was stirred at 20° C. for 2 h. After completion, the reaction mixture was concentrated under reduced pressure at 35° C. The residue was purified by flash chromatography and eluted with Petroleum ether/EtOAc=5:1 to give N-(2-chloro-6-fluorophenyl)-2-methoxyacetamide (1.8 g, Yield: 57.7%) as a white solid.

LC-MS m/z: 218.1 (M+1)⁺.

### Step 2: N-(2-chloro-6-fluoro-3-nitrophenyl)-2-methoxyacetamide

To a solution of N-(2-chloro-6-fluorophenyl)-2-methoxyacetamide (1.8 g, 8.3 mmol, 1.0 eq) in H₂SO₄ (10 mL) stirred at −55° C. was added a solution of concentrated nitric acid (0.6 mL, 11.6 mmol, 1.4 eq) in H₂SO₄ (2 mL) dropwise. The reaction mixture was stirred at −55° C. for 2 h. The resulting mixture was warmed to room temperature and poured into ice-water (30 mL) and the resulting precipitate filtered. The filter cake was dissolved in DCM (80 mL), dried over Na₂SO₄ and concentrated to give N-(2-chloro-6-fluoro-3-nitrophenyl)-2-methoxy acetamide (1.2 g, Yield: 49.4%).

LC-MS m/z: 263.1 (M+1)⁺.

### Step 3: 4-chloro-2-(methoxymethyl)-1-methyl-5-nitro-1H-benzo[d]imidazole

A mixture of N-(2-chloro-6-fluoro-3-nitrophenyl)-2-methoxyacetamide (500 mg, 1.92 mmol, 1 eq.), a solution of CH₃NH₂ in THF [2M](1.5 mL, 2.85 mmol, 1.5 eq.), and TEA (289 mg, 2.85 mmol, 1.5 eq.) in DMSO (2 mL) was stirred at room temperature for 2 hrs. The mixture was then extracted with water and ethyl acetate. The organic phase was concentrated and the residue was dissolved in DMSO. Additional TEA was added, and the mixture was stirred at 100° C. for 4 hrs. After cooling to room temperature, the mixture was diluted with water, and extracted with EtOAc (50 mL×2). The organic phase was washed with brine (50 mL×3), dried over Na₂SO₄, filtrated and concentrated in vacuo to give a residue which was purified by SGC (UV254, Petroleum ether:EtOAc=1:1) to afford 4-chloro-2-(methoxymethyl)-1-methyl-5-nitro-1H-benzo[d]imidazole (300 mg, Yield: 59.2%) as a yellow solid.

LC-MS m/z: 255.2 (M+1)⁺.

### Step 4: tert-butyl (S)-(1-(2-(methoxymethyl)-1-methyl-5-nitro-1H-benzo[d]imidazole-4-yl)pyrrolidin-3-yl)carbamate

To a solution of 4-chloro-2-(methoxymethyl)-1-methyl-5-nitro-1H-benzo[d]imidazole (120 mg, 0.47 mmol, 1.0 eq) and tert-butyl (S)-pyrrolidin-3-ylcarbamate (105 mg, 0.56 mmol, 1.2 eq) in DMSO (1.5 mL) was added diisopropylethylamine (121 mg, 0.94 mmol, 2 eq). The mixture was stirred at 100° C. for 4 hours. After cooling to room temperature, the mixture was diluted with water and extracted with EtOAc (10 mL×2). The organic phase was washed with brine (10 mL×3), dried over Na₂SO₄, filtrated and concentrated in vacuo to give a residue which was purified by SGC (UV254, Petroleum ether:EtOAc=3:1) to afford tert-butyl (S)-(1-(2-(methoxymethyl)-1-methyl-5-nitro-1H-benzo[d]imidazol-4-yl)pyrrolidin-3-yl) carbamate (100 mg, Yield: 50.5%) as a yellow solid.

LC-MS m/z: 405.2 (M+1)⁺.

### Step 5: tert-butyl (S)-(1-(5-amino-2-(methoxymethyl)-1-methyl-1H-benzo[d]imidazol-4-yl)pyrrolidin-3-yl) carbamate

To a solution of tert-butyl (S)-(1-(2-(methoxymethyl)-1-methyl-5-nitro-1H-benzo[d]imidazol-4-yl)pyrrolidin-3-yl) carbamate (548 mg, 1.0 eq) in MeOH (10 mL) was added 10% Pd/C (60 mg). The reaction was stirred for 16 hours. Filtered through a pad of Celite. The organic phase was washed with brine (20 mL×3), dried over Na₂SO₄, filtrated and concentrated in vacuo to give a residue which was purified by SGC (UV254, Petroleum ether:EtOAc=1:1) to afford compound tert-butyl (S)-(1-(5-amino-2-(methoxymethyl)-1-methyl-1H-benzo[d]imidazol-4-yl)pyrrolidin-3-yl) carbamate (300 mg, Yield: 59.2%) as a yellow solid.

LC-MS m/z: 376.2 (M+1)⁺.

### Step 6: tert-butyl(S)-(1-(5-(2-(2,6-difluorophenyl)-3-oxo-2,3-dihydropyridazine-4-carboxamido)-2-(methoxymethyl)-1-methyl-1H-benzo[d]imidazol-4-yl)pyrrolidin-3-yl)carbamate

To a mixture of tert-butyl (S)-(1-(5-amino-2-(methoxymethyl)-1-methyl-1H-benzo[d]imidazol-4-yl)pyrrolidin-3-yl)carbamate (80 mg, 0.45 mmol, 1.0 eq) and 2-(2,6-difluorophenyl)-3-oxo-2,3-dihydropyridazine-4-carboxylic acid (50 mg, 0.18 mmol, 1 eq) in DMF (2 ml) were added HATU (85.6 mg, 0.22 mmol, 1.5 eq) and DIPEA (0.29 mL, 1.35 mmol, 3.0 eq). The resulting mixture was stirred at 50° C. for 2 hours. After cooling to room temperature, the reaction was quenched with water (50 mL) and extracted with EtOAc (10 mL×3). The combined organic phase was washed with brine (50 mL), dried over Na₂SO₄, filtrated and concentrated under reduced pressure to give crude product, which was purified by SGC (UV254, Petroleum ether:EtOAc=3:1) to afford tert-butyl (S)-(1-(5-(2-(2,6-difluorophenyl)-3-oxo-2,3-dihydropyridazine-4-carboxamido)-2-(methoxymethyl)-1-methyl-1H-benzo[d]imidazol-4-yl)pyrrolidin-3-yl)carbamate (100 mg, Yield: 69.3%) as a yellow solid.

LC-MS m/z: 610.3 (M+1)⁺.

### Step 7: (S)—N-(4-(3-aminopyrrolidin-1-yl)-2-(methoxymethyl)-1-methyl-1H-benzo[d]imidazol-5-yl)-2-(2,6-difluorophenyl)-3-oxo-2,3-dihydropyridazine-4-carboxamide

To a solution of tert-butyl (S)-(1-(5-(2-(2,6-difluorophenyl)-3-oxo-2,3-dihydropyridazine-4-carboxamido)-2-(methoxymethyl)-1-methyl-1H-benzo[d]imidazol-4-yl)pyrrolidin-3-yl)carbamate (50 mg, 0.082 mmol, 1.0 eq) in DCM (3 mL) was added TFA (0.3 mL, 4M, 30.0 eq) at room temperature. The reaction mixture was stirred at 25° C. for 2 hours. The reaction mixture was concentrated and washed with tert-butyl methyl ether to afford Example 44 as a yellow solid.

LC-MS m/z: 510.2 (M+1)⁺.

¹H NMR (400 MHz, CD₃OD) δ 8.56 (d, J=8.9 Hz, 1H), 8.39 (d, J=4.2 Hz, 1H), 8.33 (d, J=4.2 Hz, 1H), 7.66-7.48 (m, 1H), 7.42-7.37 (m, 1H), 7.28-7.11 (m, 2H), 4.71-4.69 (m, 2H), 3.95-3.83 (m, 2H), 3.79 (s, 3H), 3.50-3.34 (m, 5H), 3.29-3.27 (m, 1H), 2.50-2.40 (s, 1H), 2.03-1.96 (m, 1H).

### Example 45: N-(4-((S)-3-aminopyrrolidin-1-yl)-1-methyl-2-(trifluoromethyl)-5,6-dihydro-1H-benzo[d]imidazol-5-yl)-2-(2,6-difluorophenyl)-3-oxo-2,3-dihydropyridazine-4-carboxamide

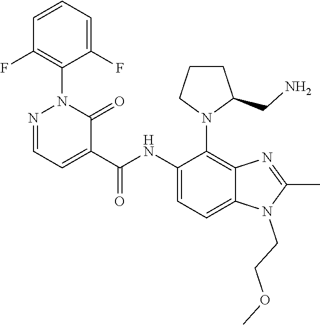 [SMILES: COCCn1c(C)nc2c(N3CCC[C@H]3CN)c(NC(=O)c3ccnn(-c4c(F)cccc4F)c3=O)ccc21]

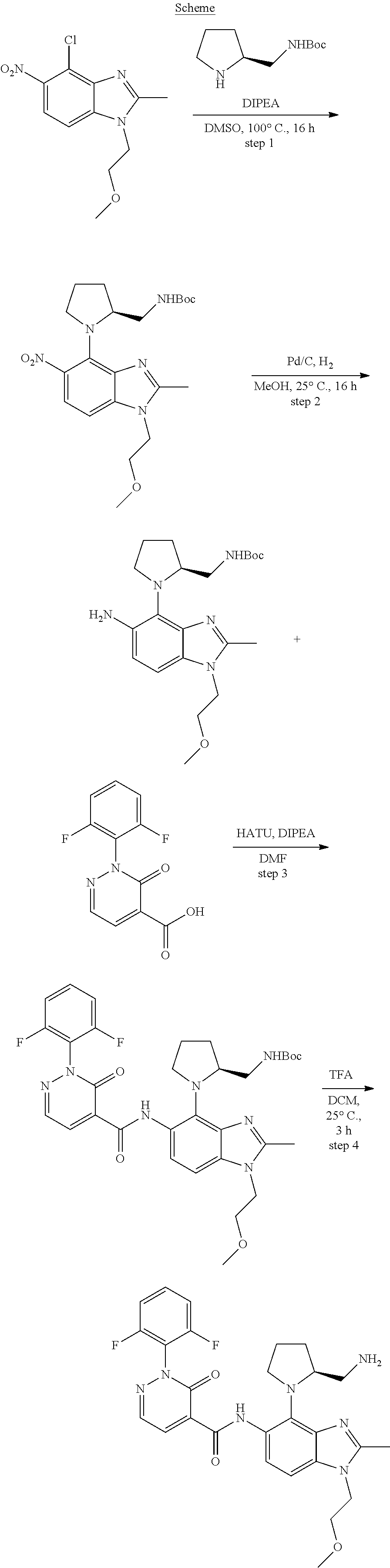

### Step 1: tert-butyl (S)-((1-(1-(2-methoxyethyl)-2-methyl-5-nitro-1H-benzo[d]imidazol-4-yl)pyrrolidin-2-yl) methyl) carbamate

A mixture of 4-chloro-l-(2-methoxyethyl)-2-methyl-5-nitro-1H-benzo[d]imidazole (270 mg, 1.00 mmol, 1.0 eq), tert-butyl (S)-(pyrrolidin-2-ylmethyl)carbamate (305 mg, 1.50 mmol, 1.5 eq) and diisopropylethylamine (518 mg, 4.00 mmol, 4 eq) in DMSO (8 mL) was stirred at 100° C. for 16 hours. After cooling to room temperature, the reaction mixture was quenched with water (5.0 mL) and extracted with EtOAc (10 mL×3). The combined organic phase was washed with brine (10 mL×3), dried over Na₂SO₄, filtrated and concentrated under reduced pressure to give a residue, which was purified by flash column chromatography (eluting with Petroleum ether/EtOAc=10/1) to afford tert-butyl (S)-((1-(1-(2-methoxyethyl)-2-methyl-5-nitro-1H-benzo[d]imidazol-4-yl)pyrrolidin-2-yl) methyl)carbamate (315 mg, Yield: 61.7%) as a yellow solid.

LC-MS m/z: 434.3 (M+1)⁺.

### Step 2: tert-butyl(S)-((1-(5-amino-1-(2-methoxyethyl)-2-methyl-1H-benzo[d]imidazol-4-yl)pyrrolidin-2-yl)methyl)carbamate

The mixture of Pd/C (40 mg, 10%) and tert-butyl (S)-((1-(1-(2-methoxyethyl)-2-methyl-5-nitro-1H-benzo[d]imidazol-4-yl)pyrrolidin-2-yl)methyl)carbamate (100 mg, 0.35 mmol) in methanol (10 mL) was hydrogenated under 15 psi for 2 hours. The reaction mixture was filtered and concentrated under reduce pressure to afford tert-butyl (S)-((1-(5-amino-1-(2-methoxyethyl)-2-methyl-1H-benzo[d]imidazol-4-yl)pyrrolidin-2-yl)methyl)carbamate (252 mg, Yield: 77.4%) as a white solid.

LC-MS m/z: 404.2 (M+1)⁺.

¹H NMR (400 MHz, d₆-DMSO) δ 6.99 (d, J=8.4 Hz, 1H), 6.66 (t, J=5.2 Hz, 1H), 6.59 (d, J=8.8 Hz, 1H), 4.58 (s, 2H), 4.20 (t, J=5.2 Hz, 2H), 3.97 (s, 1H), 3.58 (t, J=5.2 Hz, 2H), 3.21 (s, 4H), 3.17 (d, J=5.2 Hz, 1H), 2.95-2.83 (m, 1H), 2.79-2.61 (m, 1H), 2.44 (s, 3H), 2.11 (dd, J=1 Step 3: tert-butyl (S)-((1-(5-(2-(2,6-difluorophenyl)-3-oxo-2,3-dihydropyridazine-4-carboxamido)-1-(2-methoxyethyl)-2-methyl-1H-benzo[d]imidazol-4-yl)pyrrolidin-2-yl)methyl) carbamate

To a solution of tert-butyl (S)-((1-(5-amino-1-(2-methoxyethyl)-2-methyl-1H-benzo[d]imidazol-4-yl)pyrrolidin-2-yl)methyl) carbamate (139 mg, 0.35 mmol, 1.0 eq) and 2-(2,6-difluorophenyl)-3-oxo-2,3-dihydropyridazine-4-carboxylic acid (87 mg, 0.35 mmol, 1.0 eq) in DMF (2 mL) were added HATU (197 mg, 0.52 mmol, 1.5 eq) and DIPEA (0.23 mL, 1.38 mmol, 4.0 eq). The mixture was stirred at 50° C. for 16 hours. After cooling to room temperature, the mixture was diluted with water and extracted with EtOAc (5 mL×3). The organic phase was washed with brine (10 mL×3), dried over Na₂SO₄, filtrated and concentrated in vacuo to give a residue which was purified by SGC (UV254, Petroleum ether:EtOAc=1:1) to afford tert-butyl (S)-((1-(5-(2-(2,6-difluorophenyl)-3-oxo-2,3-dihydropyridazine-4-carboxamido)-1-(2-methoxyethyl)-2-methyl-1H-benzo[d]imidazol-4-yl)pyrrolidin-2-yl)methyl)carbamate (160 mg, Yield: 69.1%) as a yellow solid. 1H NMR (400 MHz, d₆-DMSO) δ 11.99 (s, 1H), 8.45 (d, J=4.4 Hz, 1H), 8.39 (s, 1H), 8.37 (d, J=4.8 Hz, 1H), 7.71 (t, J=8.0 Hz, 1H), 7.44 (t, J=8.8 Hz, 2H), 7.34 (d, J=8.8 Hz, 1H), 6 6.39 (s, 1H), 4.33 (t, J=4.8 Hz, 2H), 4.14 (s, 1H), 3.64 (s, 2H), 3.27 (s, 1H), 3.22 (s, 3H), 3.09 (q, J=7.8 Hz, 1H), 2.82-2.71 (m, 2H), 2.52 (s, 3H), 2.16-2.05 (m, 1H), 1.91-1.82 (m, 2H), 1.76-1.66 (m, 1H), 1.21 (s, 9H).

### Step 4: N-(4-((S)-3-aminopyrrolidin-1-yl)-1-methyl-2-(trifluoromethyl)-5,6-dihydro-1H-benzo[d]imidazol-5-yl)-2-(2,6-difluorophenyl)-3-oxo-2,3-dihydropyridazine-4-carboxamide

To a solution of N-(4-((S)-3-aminopyrrolidin-1-yl)-1-methyl-2-(trifluoromethyl)-5,6-dihydro-1H-benzo[d]imidazol-5-yl)-2-(2,6-difluorophenyl)-3-oxo-2,3-dihydropyridazine-4-carboxamide (80 mg, 0.13 mmol, 1.0 eq) in DCM (1 mL) was added TFA (0.5 mL). The reaction mixture was stirred at 25° C. for 3 hours. The reaction mixture was concentrated to afford Example 45 as a yellow solid.

LC-MS m/z: 538.2 (M+1)⁺.

¹H NMR (400 MHz, d₆-DMSO) δ 11.93 (s, 1H), 8.50 (d, J=4.0 Hz, 1H), 8.46 (d, J=7.2 Hz, 1H), 8.40 (d, J=4.0 Hz, 1H), 7.74 (m, 1H), 7.59 (br, 3H), 7.46 (m, 2H), 4.47 (s, 2H), 4.25 (s, 1H), 3.68 (s, 2H), 3.26 (m, 1H), 3.23 (s, 3H), 3.13-3.05 (m, 1H), 2.66 (s, 3H), 2.60 (m, 2H), 2.37-2.23 (m, 1H), 1.99-1.74 (m, 3H).

### Example 46: (S)—N-(4-(3-aminopyrrolidin-1-yl)-2-isopropyl-1-methyl-1H-benzo[d]imidazol-5-yl)-2-(2,6-difluorophenyl)-3-oxo-2,3-dihydropyridazine-4-carboxamide

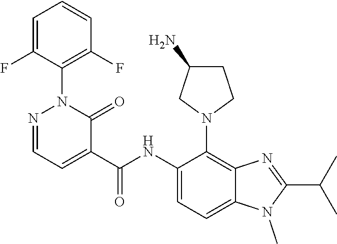 [SMILES: CC(C)c1nc2c(N3CC[C@H](N)C3)c(NC(=O)c3ccnn(-c4c(F)cccc4F)c3=O)ccc2n1C]

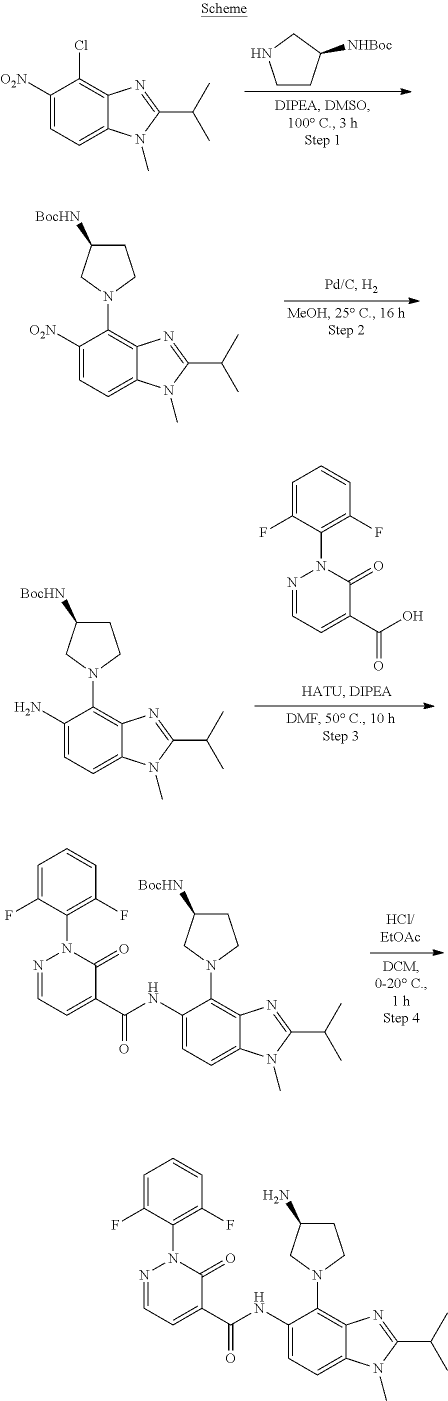

### Step 1: tert-butyl N-[(35)-1-(2-isopropyl-1-methyl-5-nitro-1,3-benzodiazol-4-yl)pyrrolidin-3-yl]carbamate

A mixture of 4-chloro-2-isopropyl-1-methyl-5-nitro-1H-benzo[d]imidazole (100 mg, 0.39 mmol, 1.0 eq), diisopropylethylamine (130 mL, 0.79 mmol, 2 eq) and tert-butyl (S)-pyrrolidin-3-ylcarbamate (88 mg, 0.43 mmol, 1.2 eq) in DMSO was stirred at 100° C. for 4 hours. After cooling to room temperature, the reaction mixture was quenched with water and extracted with EtOAc (10 mL×2). The combined organic phase was washed with brine (10 mL×3), dried over Na₂SO₄, filtrated and concentrated under reduced pressure to give a residue, which was purified by SGC (UV254, Petroleum ether:EtOAc=3:1) to afford tert-butyl N-[(35)-1-(2-isopropyl-1-methyl-5-nitro-1,3-benzodiazol-4-yl) pyrrolidin-3-yl]carbamate (150 mg, Yield: 89.6%) as a yellow solid.

LC-MS m/z: 404.1 (M+1)⁺.

### Step 2: tert-butyl (S)-(1-(5-amino-2-(methoxymethyl)-1-methyl-1H-benzo[d]imidazol-4-yl)pyrrolidin-3-yl) carbamate

The mixture of Pd/C (79 mg, 0.25 mmol, 10%) and tert-butyl N-[(35)-1-(2-isopropyl-1-methyl-5-nitro-1,3-benzodiazol-4-yl) pyrrolidin-3-yl]carbamate (150 mg, 0.37 mmol) in methanol (5 mL) was hydrogenated under 15 psi for 2 hours. The reaction mixture was filtered and concentrated to afford tert-butyl (S)-(1-(5-amino-2-(methoxymethyl)-1-methyl-1H-benzo[d]imidazol-4-yl)pyrrolidin-3-yl)carbamate (80 mg, Yield: 46.1%) as a white solid.

LC-MS m/z: 374.2 (M+1)⁺.

### Step 3: tert-butyl (S)-(1-(5-(2-(2,6-difluorophenyl)-3-oxo-2,3-dihydropyridazine-4-carboxamido)-2-isopropyl-1-methyl-1H-benzo[d]imidazol-4-yl)pyrrolidin-3-yl)carbamate

To a solution of tert-butyl (S)-(1-(5-amino-2-isopropyl-1-methyl-1H-benzo [d]imidazol-4-yl)pyrrolidin-3-yl)carbamate (100 mg, 0.27 mmol, 1.0 eq) and 2-(2,6-difluorophenyl)-3-oxo-2,3-dihydropyridazine-4-carboxylic acid (67 mg, 0.27 mmol, 1.0 eq) in DMF (2.0 mL) were added HATU (122 mg, 0.32 mmol, 1.2 eq) and DIPEA (0.2 mL, 1.33 mmol, 5.0 eq). The reaction mixture was stirred at 50° C. for 10 hours. After cooling to room temperature, the reaction was quenched with water (10 mL) and extracted with EtOAc (10 mL×3). The combined organic phase was washed with brine (10 mL×3), dried over anhydrous Na₂SO₄, filtrated and concentrated under reduced pressure to give crude product, which was purified by flash column chromatography (eluting with DCM/MeOH=25/1) to afford tert-butyl(S)-(1-(5-(2-(2,6-difluorophenyl)-3-oxo-2,3-dihydropyridazine-4-carboxamido)-2-isopropyl-1-methyl-1H-benzo[d]imidazol-4-yl)pyrrolidin-3-yl)carbamate (100 mg, Yield: 58.4%) as a yellow solid.

LC-MS m/z: 608.7 (M+1)⁺.

### Step 4: (S)—N-(4-(3-aminopyrrolidin-1-yl)-2-isopropyl-1-methyl-1H-benzo[d]imidazol-5-yl)-2-(2,6-difluorophenyl)-3-oxo-2,3-dihydropyridazine-4-carboxamide

To a solution of tert-butyl (S)-(1-(5-(2-(2,6-difluorophenyl)-3-oxo-2,3-dihydropyridazine-4-carboxamido)-2-isopropyl-1-methyl-1H-benzo[d]imidazol-4-yl)pyrrolidin-3-yl)carbamate (100 mg, 0.16 mmol) in DCM (2.0 mL) was added a solution of hydrogen chloride in 1,4-dioxane (1.0 mL, 4 M). The reaction mixture was stirred at 25° C. for 1 hour. The reaction mixture was concentrated and washed with tert-butyl methyl ether to afford Example 46 hydrochloride (40 mg, Yield: 42.9%) as a yellow solid.

LC-MS m/z: 508.7 (M+1)⁺.

¹H NMR: (400 MHz, CD₃OD) δ 8.60 (br, 1H), 8.38 (s, 1H), 8.32 (s, 1H), 7.64-7.53 (m, 1H), 7.47 (br, 1H), 7.20 (t, J=8.8 Hz, 2H), 3.93 (s, 1H), 3.83 (s, 3H), 3.80-3.74 (m, 1H), 3.44 (s, 1H), 3.40 (s, 2H), 3.26 (s, 1H), 2.47 (s, 1H), 2.07 (s, 1H), 1.41 (s, 6H).

### Example 47: (S)—N-(4-(3-aminopyrrolidin-1-yl)-2-methyl-1-(tetrahydro-2H-pyran-4-yl)-1H-benzo[d]imidazol-5-yl)-2-(2,6-difluorophenyl)-3-oxo-2,3-dihydropyridazine-4-carboxamide trifluoroacetate

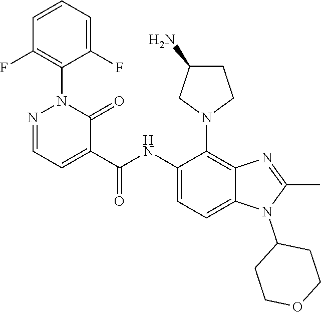 [SMILES: Cc1nc2c(N3CC[C@H](N)C3)c(NC(=O)c3ccnn(-c4c(F)cccc4F)c3=O)ccc2n1C1CCOCC1]

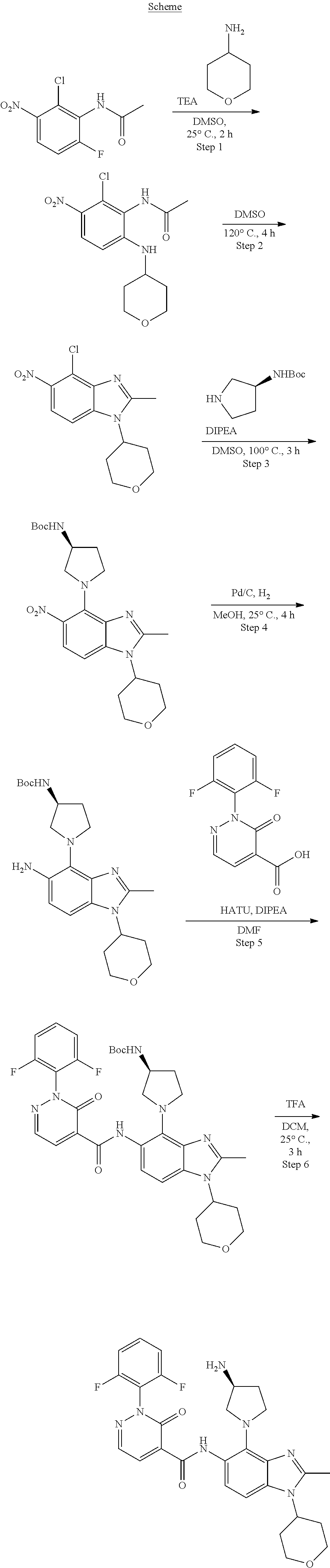

### Step 1: N-(2-chloro-3-nitro-6-((tetrahydro-2H-pyran-4-yl)amino)phenyl)acetamide

A mixture of N-(2-chloro-6-fluoro-3-nitrophenyl)acetamide (500 mg, 2.15 mmol, 1 eq), tetrahydro-2H-pyran-4-amine (326 mg, 3.22 mmol, 1.5 eq) and triethylamine (0.45 mL, 3.22 mmol, 1.5 eq) in DMSO (3 mL) was stirred at 25° C. for 2 hours. After the reaction was completed, the reaction was quenched with water (20 mL) and extracted with DCM (20 mL×3). The combined organic phase was washed with brine (20 mL×3), dried over Na₂SO₄, filtrated and concentrated under reduced pressure to afford N-(2-chloro-3-nitro-6-((tetrahydro-2H-pyran-4-yl)amino) phenyl)acetamide (370 mg, Yield: 54.9%) without further purification as a yellow solid.

LC-MS m/z: 313.9 (M+1)⁺.

### Step 2: 4-chloro-2-methyl-5-nitro-1-(tetrahydro-2H-pyran-4-yl)-1H-benzo[d]imidazole

A mixture of N-(2-chloro-3-nitro-6-((tetrahydro-2H-pyran-4-yl)amino)phenyl)acetamide (370 mg, 1.19 mmol, 1 eq) and triethylamine (0.25 mL, 1.77 mmol, 1.5 eq) in DMSO (3 mL) was stirred at 120° C. for 4 hours. After cooling to room temperature, the reaction was quenched with water (20 mL) and extracted with DCM (20 mL×3). The combined organic phase was washed with brine (20 mL×3), dried over Na₂SO₄, filtrated and concentrated under reduced pressure to give a residue, which was purified by flash column chromatography (eluting with DCM/MeOH=19/1) to afford 4-chloro-2-methyl-5-nitro-1-(tetrahydro-2H-pyran-4-yl)-1H-benzo[d]imidazole (320 mg, Yield: 40.3%) as a yellow solid.

LC-MS m/z: 295.9 (M+1)⁺.

### Step 3: tert-butyl (S)-(1-(2-methyl-5-nitro-1-(tetrahydro-2H-pyran-4-yl)-1H-benzo[d]imidazol-4-yl)pyrrolidin-3-yl)carbamate

A mixture of 4-chloro-2-methyl-5-nitro-1-(tetrahydro-2H-pyran-4-yl)-1H-benzo[d]imidazole (320 mg, 1.08 mmol, 1 eq), tert-butyl (S)-pyrrolidin-3-yl carbamate (302 mg, 1.62 mmol, 1.5 eq) and diisopropylethylamine (0.28 mL, 1.62 mmol, 1.5 eq) in DMSO (3 mL) was stirred at 100° C. for 3 hours. After cooling to room temperature, the reaction was quenched with water (20 mL) and extracted with EtOAc (20 mL×3). The combined organic phase was washed with brine (20 mL×3), dried over Na₂SO₄, filtrated and concentrated under reduced pressure to give a residue, which was purified by flash column chromatography (eluting with Petroleum ether/EtOAc=13/7) to afford tert-butyl (S)-(1-(2-methyl-5-nitro-1-(tetrahydro-2H-pyran-4-yl)-1H-benzo[d]imidazol-4-yl)pyrrolidin-3-yl)carbamate (345 mg, Yield: 68.0%) as a yellow solid.

LC-MS m/z: 446.1 (M+1)⁺.

### Step 4: tert-butyl (S)-(1-(5-amino-2-methyl-1-(tetrahydro-2H-pyran-4-yl)-1H-benzo[d]imidazol-4-yl)pyrrolidin-3-yl)carbamate

The mixture of Pd/C (170 mg, 10%) and tert-butyl (S)-(1-(2-methyl-5-nitro-1-(tetrahydro-2H-pyran-4-yl)-1H-benzo[d]imidazol-4-yl)pyrrolidin-3-yl)carbamate (345 mg, 0.77 mmol) in methanol (3 mL) was hydrogenated under 15 psi for 4 hours. The reaction mixture was filtered and concentrated under reduce pressure to afford tert-butyl (S)-(1-(5-amino-2-methyl-1-(tetrahydro-2H-pyran-4-yl)-1H-benzo[d]imidazol-4-yl)pyrrolidin-3-yl)carbamate (337.0 mg, Yield: 94.3%) as a gray solid.

LC-MS m/z: 416.2 (M+1)⁺.

### Step 5: tert-butyl (S)-(1-(5-(2-(2,6-difluorophenyl)-3-oxo-2,3-dihydropyridazine-4-carboxamido)-2-methyl-1-(tetrahydro-2H-pyran-4-yl)-1H-benzo[d]imidazol-4-yl)pyrrolidin-3-yl)carbamate

To a solution of tert-butyl (S)-(1-(5-amino-2-methyl-1-(tetrahydro-2H-pyran-4-yl)-1H-benzo[d]imidazol-4-yl)pyrrolidin-3-yl)carbamate (100 mg, 0.24 mmol, 1.0 eq) and 2-(2,6-difluorophenyl)-3-oxo-2,3-dihydropyridazine-4-carboxylic acid (61 mg, 0.24 mmol, 1.0 eq) in DMF (2 mL) were added HATU (110 mg, 0.29 mmol, 1.2 eq) and DIPEA (0.21 mL, 1.20 mmol, 5.0 eq). The reaction mixture was stirred at 50° C. for 2 hours. After cooling to room temperature, the reaction was quenched with water (20 mL) and extracted with EtOAc (20 mL×3). The combined organic phase was washed with brine (20 mL×3), dried over anhydrous Na₂SO₄, filtrated and concentrated under reduced pressure to give crude product, which was purified by flash column chromatography (eluting with DCM/MeOH=19/1)) to afford tert-butyl (S)-(1-(5-(2-(2,6-difluorophenyl)-3-oxo-2,3-dihydropyridazine-4-carboxamido)-2-methyl-1-(tetrahydro-2H-pyran-4-yl)-1H-benzo[d]imidazol-4-yl)pyrrolidin-3-yl)carbamate (85 mg, Yield: 51.6%) as an orange solid.

LC-MS m/z: 650.3 (M+1)⁺.

### Step 6: (S)—N-(4-(3-aminopyrrolidin-1-yl)-2-methyl-1-(tetrahydro-2H-pyran-4-yl)-1H-benzo[d]imidazol-5-yl)-2-(2,6-difluorophenyl)-3-oxo-2,3-dihydropyridazine-4-carboxamide

To a solution of tert-butyl (S)-(1-(5-(2-(2,6-difluorophenyl)-3-oxo-2,3-dihydro pyridazine-4-carboxamido)-2-methyl-1-(tetrahydro-2H-pyran-4-yl)-1H-benzo[d]imidazol-4-yl)pyrrolidin-3-yl)carbamate (85 mg, 0.14 mmol) in DCM (2 mL) was added trifluoroacetic acid (1 mL). The reaction mixture was stirred at 25° C. for 3 hours. Water (5 mL) was added and extracted with DCM (5 mL). The combined organic phase was concentrated under reduced pressure to afford

### Example 47 (80.9 mg, Yield: 90%) as a Yellow Solid

LC-MS m/z: 550.2 (M+1)⁺.

¹H NMR (400 MHz, CD₃OD) δ 8.66 (d, J=9.1 Hz, 1H), 8.50 (d, J=4.2 Hz, 1H), 8.44 (d, J=4.2 Hz, 1H), 7.77 (d, J=9.2 Hz, 1H), 7.75-7.67 (m, 1H), 7.36-7.30 (m, 2H), 4.75-4.73 (m, 1H), 4.16-4.14 (m, 2H), 4.01-3.99 (m, 1H), 3.88 (dd, J=11.0, 5.3 Hz, 1H), 3.67-3.65 (m, 2H), 3.51-3.46 (m, 2H), 3.28-3.26 (m, 1H), 2.79 (s, 3H), 2.62-2.58 (m, 3H), 2.13-2.04 (m, 1H), 1.91-1.89 (m, 2H).

### Example 48: N-{4-[(35)-3-aminopyrrolidin-1-yl]-1-cyclopropyl-2-methyl-1,3-benzodiazol-5-yl}-2-(2,6-difluoro phenyl)-3-oxopyridazine-4-carboxamide

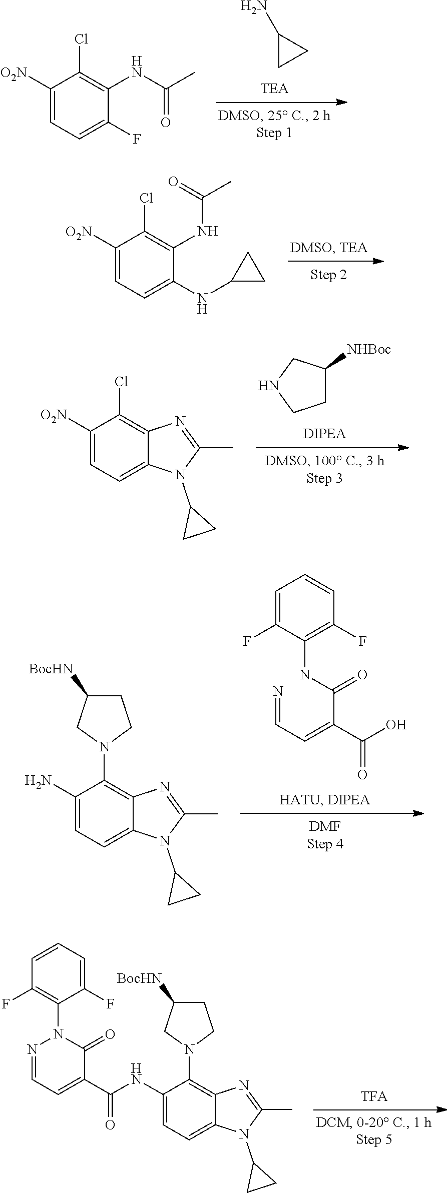

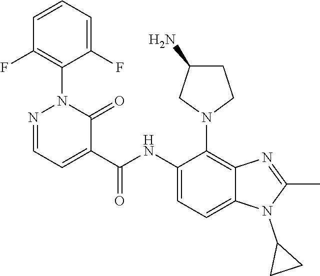 [SMILES: Cc1nc2c(N3CC[C@H](N)C3)c(NC(=O)c3ccnn(-c4c(F)cccc4F)c3=O)ccc2n1C1CC1]

### Step 1: N-(2-chloro-6-(cyclopropylamino)-3-nitrophenyl)acetamide

A mixture of N-(2-chloro-6-fluoro-3-nitrophenyl)acetamide (500 mg, 2.15 mmol, 1.0 eq), cyclopropanamine (184 mg, 3.22 mmol, 1.5 eq) and triethylamine (0.45 mL, 3.22 mmol, 1.5 eq) in DMSO (3 mL) was stirred at 25° C. for 2 hours. After the reaction was completed, the reaction was quenched with water (20 mL) and extracted with EtOAc (20 mL×3). The combined organic phase was washed with brine (20 mL×3), dried over Na₂SO₄, filtrated and concentrated under reduced pressure to afford N-(2-chloro-6-(cyclopropylamino)-3-nitrophenyl) acetamide (451 mg, Yield: 78%) without further purification as a yellow solid.

LC-MS m/z: 270.0 (M+1)⁺.

### Step 2: 4-chloro-1-cyclopropyl-2-methyl-5-nitro-1H-benzo[d]imidazole

A mixture of N-(2-chloro-6-(cyclopropylamino)-3-nitrophenyl)acetamide (451 mg, 1.67 mmol, 1.0 eq) and triethylamine (0.35 mL, 2.50 mmol, 1.5 eq) in DMSO (3 mL) was stirred at 120° C. for 4 hours. After cooling to room temperature, the reaction was quenched with water (20 mL) and extracted with EtOAc (20 mL×3). The combined organic phase was washed with brine (20 mL×3), dried over Na₂SO₄, filtrated and concentrated under reduced pressure to give a residue, which was purified by flash column chromatography (eluting with Petroleum ether/EtOAc (=97/3)) to afford 4-chloro-1-cyclopropyl-2-methyl-5-nitro-1H-benzo[d]imidazole (294 mg, Yield: 48.9%) as a light yellow solid.

LC-MS m/z: 251.9 (M+1)⁺.

### Step 3: tert-butyl (S)-(1-(1-cyclopropyl-2-methyl-5-nitro-1H-benzo[d]imidazol-4-yl) pyrrolidin-3-yl) carbamate

A mixture of 4-chloro-1-cyclopropyl-2-methyl-5-nitro-1H-benzo[d]imidazole (150 mg, 0.60 mmol, 1.0 eq), tert-butyl (S)-pyrrolidin-3-ylcarbamate (167 mg, 0.89 mmol, 1.48 eq) and diisopropylethylamine (0.16 mL, 0.89 mmol, 1.48 eq) in DMSO (3 mL) was stirred at 100° C. for 3 hours. After cooling to room temperature, the reaction was quenched with water (20 mL) and extracted with EtOAc (20 mL×3). The combined organic phase was washed with brine (20 mL×3), dried over Na₂SO₄, filtrated and concentrated under reduced pressure to give a residue, which was purified by flash column chromatography (eluting with Petroleum ether/EtOAc=4/1) to afford tert-butyl (S)-(1-(1-cyclopropyl-2-methyl-5-nitro-1H-benzo[d]imidazol-4-yl)pyrrolidin-3-yl) carbamate (110 mg, Yield: 30.7%) as a yellow solid.

LC-MS m/z: 402.0 (M+1)⁺.

### Step 4: tert-butyl (S)-(1-(5-amino-1-cyclopropyl-2-methyl-1H-benzo[d]imidazol-4-yl)pyrrolidin-3-yl)carbamate

The mixture of Pd/C (55 mg, 10%) and tert-butyl (S)-(1-(1-cyclopropyl-2-methyl-5-nitro-1H-benzo[d]imidazol-4-yl)pyrrolidin-3-yl)carbamate (110 mg, 0.27 mmol) in methanol (3 mL) was hydrogenated under 15 psi for 4 hours. The reaction mixture was filtered and concentrated under reduce pressure to afford tert-butyl (S)-(1-(5-amino-1-cyclopropyl-2-methyl-1H-benzo[d]imidazol-4-yl)pyrrolidin-3-yl)carbamate (82 mg, Yield: 72.5%) as a gray solid.

LC-MS m/z: 372.2 (M+1)⁺.

### Step 4: tert-butyl (S)-(1-(1-cyclopropyl-5-(2-(2,6-difluorophenyl)-3-oxo-2,3-dihydropyridazine-4-carboxamido)-2-methyl-1H-benzo[d]imidazol-4-yl)pyrrolidin-3-yl)carbamate

To a solution of tert-butyl (S)-(1-(5-amino-1-cyclopropyl-2-methyl-1H-benzo[d]imidazol-4-yl)pyrrolidin-3-yl)carbamate (82 mg, 0.22 mmol, 1.0 eq) and 2-(2,6-difluorophenyl)-3-oxo-2,3-dihydropyridazine-4-carboxylic acid (56 mg, 0.22 mmol, 1.0 eq) in DMF (2 mL) were added HATU (101 mg, 0.26 mmol, 1.18 eq) and DIPEA (0.19 mL, 1.10 mmol, 5.0 eq) at room temperature. The reaction temperature was increased to 50° C., and the reaction was stirred at 50° C. for 2 hours. After cooling to room temperature, the reaction was quenched with water (20 mL) and extracted with EtOAc (20 mL×3). The combined organic phase was washed with brine (20 mL×3), dried over anhydrous Na₂SO₄, filtrated and concentrated under reduced pressure to give crude product, which was purified by flash column chromatography (eluting with Petroleum ether/EtOAc=7/3) to afford tert-butyl (S)-(1-(1-cyclopropyl-5-(2-(2,6-difluorophenyl)-3-oxo-2,3-dihydropyridazine-4-carboxamido)-2-methyl-1H-benzo[d]imidazol-4-yl)pyrrolidin-3-yl)carbamate (92 mg, Yield: 65.4%) as a yellow solid.

LC-MS m/z: 606.2 (M+1)⁺.

### Step 5: (S)—N-(4-(3-aminopyrrolidin-1-yl)-1-cyclopropyl-2-methyl-1H-benzo[d]imidazol-5-yl)-2-(2,6-difluorophenyl)-3-oxo-2,3-dihydropyridazine-4-carboxamide

To a solution tert-butyl (S)-(1-(1-cyclopropyl-5-(2-(2,6-difluorophenyl)-3-oxo-2,3-dihydropyridazine-4-carboxamido)-2-methyl-1H-benzo[d]imidazol-4-yl)pyrrolidin-3-yl)carbamate (87 mg, 0.14 mmol) in DCM (2 mL) was added trifluoroacetic acid (1 mL) at 25° C. The reaction mixture was stirred at 25° C. for 3 hours. Water (5 mL) was added and the mixture was extracted with DCM (5 mL). The aqueous phase was freeze-dried to afford Example 48 as a yellow solid.

LC-MS m/z: 506.2 (M+1)⁺.

¹H NMR (400 MHz, CD₃OD) δ 8.74 (d, J=9.1 Hz, 1H), 8.51-8.32 (m, 2H), 7.72 (d, J=8.9 Hz, 1H), 7.66-7.53 (m, 1H), 7.22 (m, 2H), 3.94 (s, 1H), 3.69 (m, 1H), 3.52-3.32 (m, 2H), 3.37 (m, 2H), 2.91 (s, 3H), 2.64 (m, 1H), 2.09 (m, 1H), 1.42 (d, J=6.0 Hz, 2H), 1.23 (s, 2H).

### Example 49: (S)—N-(4-(3-aminopyrrolidin-1-yl)-1,2-dimethyl-1H-benzo[d]imidazol-5-yl)-2-(2-methoxyphenyl)-3-oxo-2,3-dihydropyridazine-4-carboxamide

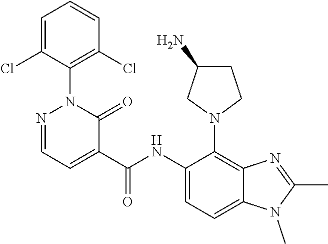 [SMILES: Cc1nc2c(N3CC[C@H](N)C3)c(NC(=O)c3ccnn(-c4c(Cl)cccc4Cl)c3=O)ccc2n1C]

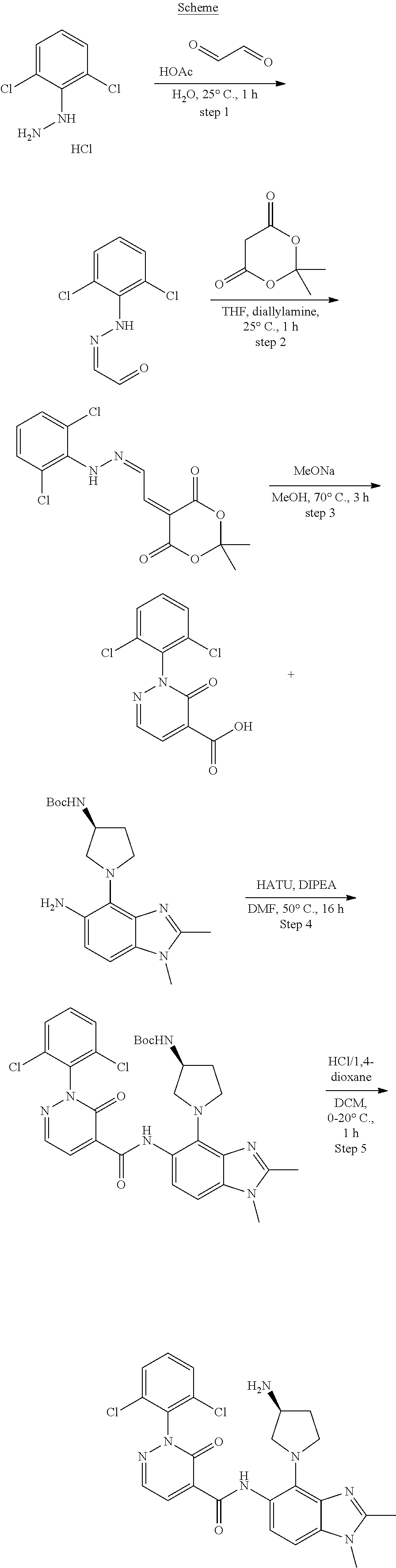

### Step 1: (Z)-2-(2-(2,6-dichlorophenyl)hydrazineylidene)acetaldehyde

To a mixture of glyoxal (80 mL, 40% in water) and THF (40 mL) was added dropwise a solution of (2,6-dichlorophenyl)hydrazine hydrochloride (8.0 g, 38 mmol, 1 eq) in H₂O/HOAc (volume ratio=5/1, 48 mL) at 25° C. The reaction mixture was continued to stir for 1 hour at the same temperature. LCMS analysis of the reaction mixture showed full conversion to the desired product. The mixture quenched by the addition of saturated NaHCO₃ and extracted with ethyl acetate (3×100 mL). The combined organic layers were washed with brine (300 mL), dried over Na₂SO₄ and filtered. The filtrate was concentrated and the residue was purified by silica gel column chromatography (Petroleum ether:Ethyl acetate, from 0 to 9%) to afford (Z)-2-(2-(2,6-dichlorophenyl)hydrazineylidene)acetaldehyde (4 g, Yield: 44.3%) as a yellow solid.

LC-MS m/z: 218.9 (M+1)⁺.

### Step 2: (Z)-5-(2-(2-(2,6-dichlorophenyl)hydrazineylidene)ethylidene)-2,2-dimethyl-1,3-dioxane-4,6-dione

To a solution of (Z)-2-(2-(2,6-dichlorophenyl)hydrazineylidene)acetaldehyde (2 g, 9.2 mmol, 1 eq) and 2,2-dimethyl-1,3-dioxane-4,6-dione (1.46 g, 10 mmol, 1.1 eq) in THF (30 mL) was added dropwise diallylamine (2 mL) at 25° C. The reaction mixture was continued to stir at the same temperature for 1 hour. LCMS analysis of the reaction mixture showed full conversion to the desired product. The reaction mixture was concentrated under reduced pressure to remove most of the solvent, then diluted with H₂O and extracted with ethyl acetate (30 mL×3). The combined organic layers were washed with brine (100 mL), dried over Na₂SO₄ and concentrated to afford (Z)-5-(2-(2-(2,6-dichlorophenyl)hydrazineylidene)ethylidene)-2,2-dimethyl-1,3-dioxane-4,6-dione (2.5 g, Yield: 47.8%) as a black solid directly used for next step. Step 3: 2-(2,6-dichlorophenyl)-3-oxo-2,3-dihydropyridazine-4-carboxylic acid

To a solution of (Z)-5-(2-(2-(2,6-dichlorophenyl)hydrazineylidene)ethylidene)-2,2-dimethyl-1,3-dioxane-4,6-dione (2.5 g, 7.3 mmol, 1 eq) in MeOH (50 mL) was added NaOMe (0.59 g, 10.9 mmol, 1.5 eq) under N₂. The reaction mixture was stirred at 70° C. LCMS analysis of the reaction mixture showed full conversion to the desired product. The mixture was concentrated under reduced pressure to remove most of the solvent, diluted with H₂O (100 mL), and washed with ethyl acetate (200 mL). The water phase was adjusted to pH=3 with citric acid and extracted with ethyl acetate (30 mL×3). The combined organic layers were washed with brine (100 mL), dried over Na₂SO₄ and filtered. The filtrate was concentrated under reduced pressure and the residue was purified by silica gel column chromatography (DCM:MeOH, from 0 to 1%) to afford 2-(2,6-dichlorophenyl)-3-oxo-2,3-dihydropyridazine-4-carboxylic acid as a yellow solid (0.6 g, Yield: 26.0%).

LC-MS m/z: 283 (M−1)⁻.

¹H NMR (400 MHz, d₆-DMSO): δ 13.77 (s, 1H), 8.27 (d, J=4.0 Hz, 1H), 7.95 (d, J=4.0 Hz, 1H), 7.72 (d, J=8.4 Hz, 2H), 7.61-7.57 (m, 1H).

### Step 4: tert-butyl N-[(35)-1-{5-[2-(2,6-dichlorophenyl)-3-oxopyridazine-4-amido]-1,2-dimethyl-1,3-benzodiazol-4-yl}pyrrolidin-3-yl]carbamate

To a solution of tert-butyl N-[(35)-1-(5-amino-1,2-dimethyl-1,3-benzodiazol-4-yl)pyrrolidin-3-yl]carbamate (50 mg, 0.14 mmol, 1.0 eq) and 2-(2,6-dichlorophenyl)-3-oxopyridazine-4-carboxylic acid (41 mg, 0.14 mmol, 1.0 eq) in DMF (0.5 mL) were added HATU (66 mg, 0.17 mmol, 1.2 eq) and DIPEA (0.12 mL, 0.72 mmol, 5.0 eq). The reaction mixture was stirred at 50° C. for 16 hours. After cooling to room temperature, the reaction was quenched with water (5 mL) and extracted with EtOAc (10 mL×3). The combined organic phase was washed with brine (10 mL×3), dried over anhydrous Na₂SO₄, filtrated and concentrated under reduced pressure to give crude product, which was purified by flash column chromatography (eluting with DCM/MeOH=97/3) to afford tert-butyl N-[(35)-1-{5-[2-(2,6-dichlorophenyl)-3-oxopyridazine-4-amido]-1,2-dimethyl-1,3-benzodiazol-4-yl}pyrrolidin-3-yl]carbamate (76 mg, Yield: 85.7%) as an orange solid.

LC-MS m/z: 612.18 (M+1)⁺.

### Step 5: N-{4-[(35)-3-aminopyrrolidin-1-yl]-1,2-dimethyl-1,3-benzodiazol-5-yl}-2-(2,6-dichlorophenyl)-3-oxopyridazine-4-carboxamide hydrochloride

To a solution of tert-butyl N-[(35)-1-{5-[2-(2,6-dichlorophenyl)-3-oxopyridazine-4-amido]-1,2-dimethyl-1,3-benzodiazol-4-yl}pyrrolidin-3-yl]carbamate (76 mg, 0.12 mmol) in DCM (2.0 mL) was added a solution of hydrogen chloride in 1,4-dioxane (0.2 mL, 4 M). The reaction mixture was stirred at 25° C. for 1 hour. The reaction mixture was concentrated and washed with tert-butyl methyl ether to afford Example 49 hydrochloride (41.5 mg, Yield: 55.9%) as a yellow solid.

LC-MS m/z: 512.1 (M+1)⁺.

¹H NMR (400 MHz, CD₃OD) δ 8.89 (d, J=5.5 Hz, 1H), 8.61 (d, J=4.2 Hz, 1H), 8.56 (d, J=4.2 Hz, 1H), 7.80 (d, J=9.0 Hz, 1H), 7.75-7.72 (m, 2H), 7.69-7.61 (m, 1H), 4.10-4.07 (m, 1H), 4.01 (s, 3H), 3.87-3.83 (m, 1H), 3.56-3.52 (m, 1H), 3.44-3.36 (m, 1H), 2.93 (s, 3H), 2.74-2.70 (m, 1H), 2.17-1.97 (m, 1H).

### Example 50: tert-butyl-N-[(35)-1-{5-[2-(5-fluoro-2-methoxyphenyl)-3-oxopyridazine-4-amido]-1,2-dimethyl-1,3-benzodiazol-4-yl}pyrrolidin-3-yl]carbamate hydrochloride

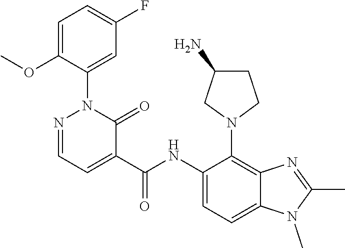

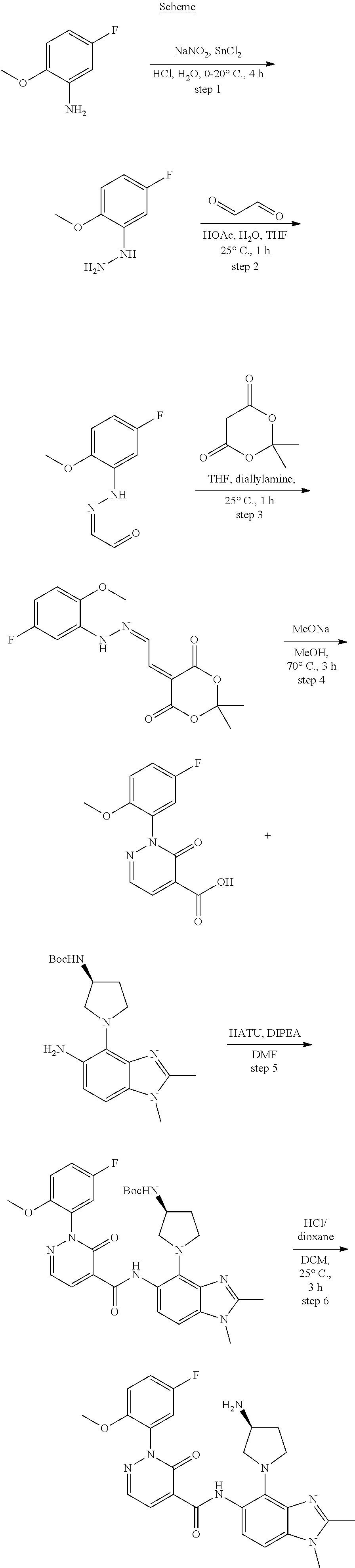

### Step 1: (5-fluoro-2-methoxyphenyl)hydrazine

To a solution of 5-fluoro-2-methoxyaniline (1 g, 7.7 mmol, 1 eq) in 5M HCl (10 mL) was added NaNO₂ (0.64 g, 9.2 mmol, 1.3 eq) slowly at 0^{˜}5° C. After stirring for 2 hours at 0^{˜}5° C., SnCl₂ (3.37 g, 17.7 mmol, 2.5 eq) was added to the reaction at 0^{˜}5° C. The reaction mixture was warmed to 25° C. and stirred for 2 hours. LCMS analysis of the reaction mixture showed full conversion to the desired product. The reaction mixture was quenched by the addition of 1M NaOH and extracted with ethyl acetate (30 mL×3). The combined organic layers were washed with brine 50 mL, dried over Na₂SO₄ and filtered. The filtrate was concentrated to give (5-fluoro-2-methoxyphenyl)hydrazine (0.8 g, Yield: 57.8%) as a black solid.

LC-MS m/z: 157.1 (M+1)⁺.

### Step 2: (Z)-2-(2-(5-fluoro-2-methoxyphenyl)hydrazineylidene)acetaldehyde

To a mixture of glyoxal (10 mL, 40% in water) and THF (5 mL) was added a solution of (5-fluoro-2-methoxyphenyl)hydrazine (0.8 g, 5.1 mmol, 1 eq) in H₂O/HOAc (volume ratio=1/1, 10 mL) at 25° C. The reaction mixture was continued to stir at the same temperature for 1 hour. LCMS analysis of the reaction mixture showed full conversion to the desired product. The mixture was quenched by the addition of saturated NaHCO₃, and extracted with ethyl acetate (30 mL×3). The combined organic layers were washed with brine (50 mL), dried over Na₂SO₄ and filtered. The filtrate was concentrated and the residue was purified by silica gel column chromatography (Petroleum ether:Ethyl acetate, from 0 to 10%) to afford (Z)-2-(2-(5-fluoro-2-methoxyphenyl)hydrazineylidene)acetaldehyde (0.8 g, Yield: 72.6%) as a yellow solid.

LC-MS m/z: 197.1 (M+1)⁺.

### Step 3: (Z)-5-(2-(2-(5-fluoro-2-methoxyphenyl)hydrazineylidene)ethylidene)-2,2-dimethyl-1,3-dioxane-4,6-dione

To a solution of (Z)-2-(2-(5-fluoro-2-methoxyphenyl)hydrazineylidene)acetaldehyde (0.8 g, 4.1 mmol, 1 eq) and 2,2-dimethyl-1,3-dioxane-4,6-dione (0.65 g, 4.5 mmol, 1.1 eq) in THF (20 mL) was added dropwise diallylamine (0.8 mL) at 25° C. The reaction mixture was continued to stir at the same temperature for 1 hour. LCMS analysis of the reaction mixture showed full conversion to the desired product. The reaction mixture was concentrated under reduced pressure to remove most of the solvent, then diluted with H₂O and extracted with ethyl acetate (20 mL×3). The combined organic layers were washed with brine (50 mL), dried over Na₂SO₄ and concentrated to afford (Z)-5-(2-(2-(5-fluoro-2-methoxyphenyl)hydrazineylidene) ethylidene)-2,2-dimethyl-1,3-dioxane-4,6-dione (1 g, Yield: 53.7%) as a black solid directly used for next step. MS (ES-API negative): 321.0 (M−1)⁻.

### Step 4: 2-(5-fluoro-2-methoxyphenyl)-3-oxo-2,3-dihydropyridazine-4-carboxylic acid

To a solution of (Z)-5-(2-(2-(5-fluoro-2-methoxyphenyl)hydrazineylidene)ethylidene)-2,2-dimethyl-1,3-dioxane-4,6-dione (1 g, 3.1 mmol, 1 eq) in MeOH (20 mL) was added NaOMe (0.25 g, 4.6 mmol, 1.5 eq) under N₂. The reaction mixture was stirred at 70° C. for 3 hours. LCMS analysis of the reaction mixture showed full conversion to the desired product. The reaction mixture was concentrated under reduced pressure to remove most of the solvent, diluted with H₂O, and washed with ethyl acetate (20 mL). The water phase was adjusted to pH=3 with citric acid, and extracted with ethyl acetate (20 mL×3). The combined organic layers were washed with brine (50 mL), dried over Na₂SO₄ and filtered. The filtrate was concentrated under reduced pressure and the residue was purified by silica gel column chromatography (DCM:MeOH, from 0 to 1%) to afford 2-(5-fluoro-2-methoxyphenyl)-3-oxo-2,3-dihydropyridazine-4-carboxylic acid (0.3 g, Yield: 32.3%) as a light brown solid.

LC-MS m/z: 264.9 (M+1)⁺.

¹H NMR (400 MHz, d₆-DMSO) δ 13.66 (s, 1H), 8.20 (d, J=4.0 Hz, 1H), 7.95 (d, J=4.0 Hz, 1H), 7.42-7.35 (m, 2H), 7.25 (dd, J=9.2, 4.8 Hz, 1H), 3.75 (s, 3H). Step 5:tert-butyl (S)-(1-(5-(2-(5-fluoro-2-methoxyphenyl)-3-oxo-2,3-dihydropyridazine-4-carboxamido)-1,2-dimethyl-1H-benzo[d]imidazol-4-yl)pyrrolidin-3-yl)carbamate

To a solution of tert-butyl (S)-(1-(5-amino-1,2-dimethyl-1H-benzo[d]imidazol-4-yl) pyrrolidin-3-yl)carbamate (157 mg, 0.45 mmol, 1.2 eq) and 2-(5-fluoro-2-methoxyphenyl)-3-oxo-2,3-dihydropyridazine-4-carboxylic acid (100 mg, 0.38 mmol, 1.0 eq) in DMF (2 mL) were added HATU (216 mg, 0.57 mmol, 1.5 eq) and N,N-diisopropylethylamine (0.19 mL, 1.14 mmol, 3.0 eq). The reaction was stirred at 50° C. for 2 hours. After cooling to room temperature, the reaction was quenched with water (50 mL) and extracted with EtOAc (10 mL×3). The combined organic phase was washed with brine (100 mL), dried over Na₂SO₄, filtrated and concentrated under reduced pressure to give crude product, which was purified by SGC (UV254, DCM:MeOH=20:1) to afford tert-butyl (S)-(1-(5-(2-(5-fluoro-2-methoxyphenyl)-3-oxo-2,3-dihydropyridazine-4-carboxamido)-1,2-dimethyl-1H-benzo[d]imidazol-4-yl)pyrrolidin-3-yl)carbamate (200 mg, Yield: 67.0%) as a light brown solid.

LC-MS m/z: 592.3 (M+1)⁺.

### Step 6: (S)—N-(4-(3-aminopyrrolidin-1-yl)-1,2-dimethyl-1H-benzo[d]imidazol-5-yl)-2-(5-fluoro-2-methoxyphenyl)-3-oxo-2,3-dihydropyridazine-4-carboxamide

To a solution of tert-butyl (S)-(1-(5-(2-(5-fluoro-2-methoxyphenyl)-3-oxo-2,3-dihydropyridazine-4-carboxamido)-1,2-dimethyl-1H-benzo[d]imidazol-4-yl)pyrrolidin-3-yl)carbamate (70 mg, 0.12 mmol, 1.0 eq) in DCM (5 mL) was added a solution of HCl in 1,4-dioxane (1 mL, 4M). The reaction mixture was stirred at 25° C. for 2 hours. The reaction mixture was concentrated and washed with DCM to afford Example 50 hydrogen chloride (26.6 mg,

Yield: 42.3%) as a yellow solid.

LC-MS m/z: 492.1 (M+1)⁺.

¹H NMR (400 MHz, d₆-DMSO) δ 12.17 (s, 1H), 8.55 (s, 1H), 8.41 (d, J=4.4 Hz, 1H), 8.36 (d, J=4.4 Hz, 1H), 8.19 (s, 2H), 7.77 (br, 1H), 7.45-7.43 (m, 2H), 7.32-7.30 (m, 1H), 3.87 (s, 3H), 3.80 (s, 3H), 3.76-3.74 (m, 1H), 3.28 (s, 2H), 2.82 (s, 2H), 2.51 (s, 3H), 2.43 (s, 1H), 1.99 (s, 1H).

### Example 51: N-(2-((1R,4R)-2,5-diazabicyclo[2.2.1]heptan-2-yl)-5-fluorophenyl)-1-(2-fluoro-6-methoxyphenyl)-2-oxo-1,2-dihydropyridine-3-carboxamide

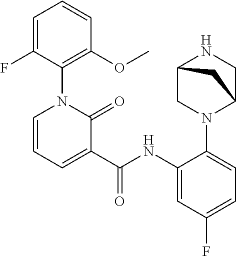 [SMILES: COc1cccc(F)c1-n1cccc(C(=O)Nc2cc(F)ccc2N2C[C@H]3C[C@@H]2CN3)c1=O]

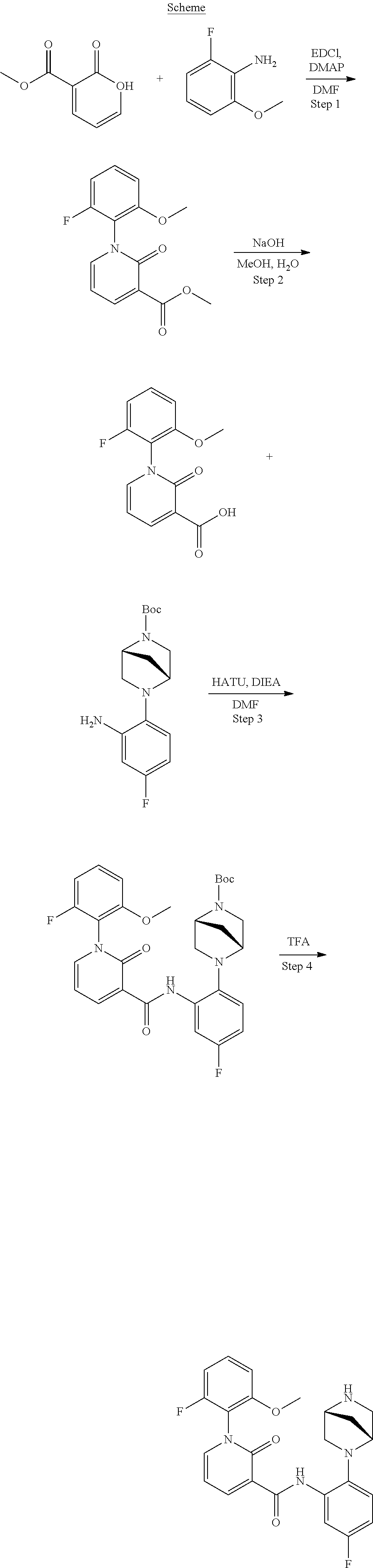

### Step 1: methyl 1-(2-fluoro-6-methoxyphenyl)-2-oxo-1,2-dihydropyridine-3-carboxylate

To a solution of 3-(methoxycarbonyl)-2-oxo-1,2-dihydropyrylium (1 g, 6.49 mmol, 1 eq.) in DMF (5 mL) was added 2-fluoro-6-methoxyaniline (0.92 g, 6.49 mmol, 1 eq.) at 0° C. After stirring for 7 hours, EDCl (2.1 g, 8.43 mmol, 1.3 eq.) and DMAP (0.2 g, 1.62 mmol, 0.25 eq.) was added to the reaction. The reaction was stirred for 16 hours at 26° C. to give a brown solution. LCMS showed the reaction was messy and the starting material was consumed up. The reaction was diluted with H₂O and extracted with EA (20 mL×3). The combined organic layers were washed with brine, dried over Na₂SO₄, filtered and concentrated to dryness. The residue was triturated with i-PrOH and filtered, and the filtrate was concentrated to afford methyl 1-(2-fluoro-6-methoxyphenyl)-2-oxo-1,2-dihydropyridine-3-carboxylate (2.3 g, crude) as brown oil which was used into next step directly.

LC-MS m/z: 278.1 (M+1)⁺.

### Step 2: 1-(2-fluoro-6-methoxyphenyl)-2-oxo-1,2-dihydropyridine-3-carboxylic acid

To a solution of methyl 1-(2-fluoro-6-methoxyphenyl)-2-oxo-1,2-dihydropyridine-3-carboxylate (6.49 mmol, 1 eq.) in MeOH (20 mL) was added a solution of NaOH (2.08 g, 51.92 mmol, 8 eq.) in H₂O (30 mL). The reaction was stirred for 1 hour at 65° C. to give a black brown solution. LCMS showed the reaction the starting material was consumed up and one peak with desired mass was detected. The reaction was treated with H₂O and washed with EA (30 mL×3). The aqueous layer was acidified with 1N HCl to pH=1^{˜}2 and extracted with EA (30 mL×3). The combined organic layers were washed with brine, dried over Na₂SO₄, filtered and concentrated to afford 1-(2-fluoro-6-methoxyphenyl)-2-oxo-1,2-dihydropyridine-3-carboxylic acid (523 mg, Yield: 30.58%) as a brown solid.

LC-MS m/z: 262.1 (M−1)⁻.

### Step 3: tert-butyl(1R,4R)-5-(4-fluoro-2-(1-(2-fluoro-6-methoxyphenyl)-2-oxo-1,2-dihydropyridine-3-carboxamido)phenyl)-2,5-diazabicyclo[2.2.1]heptane-2-carboxylate

To a solution of 1-(2-fluoro-6-methoxyphenyl)-2-oxo-1,2-dihydropyridine-3-carboxylic acid (79.35 mg, 301.47 umol, 1.5 eq.) and tert-butyl (1R,4R)-5-(2-amino-4-fluorophenyl)-2,5-diazabicyclo[2.2.1]heptane-2-carboxylate (65 mg, 200.98 umol, 1 eq.) in DMF (2 mL) was added HATU (152.84 mg, 401.96 umol, 2 eq.) and DIEA (77.93 mg, 602.95 umol, 3 eq.). The reaction was stirred for 16 hours at 26° C. to give a black brown solution. LCMS showed the reaction the starting material was consumed up and one peak with desired mass was detected. The reaction was treated with H₂O and acidified with 1N HCl to pH=4^{˜}5. The mixture was extracted with EA (10 mL×3). The combined organic layers were washed with brine, dried over Na₂SO₄, filtered and concentrated to dryness which was used into next step directly.

LC-MS m/z: 553.2 (M+1)⁺.

### Step 4: N-(2-((1R,4R)-2,5-diazabicyclo[2.2.1]heptan-2-yl)-5-fluorophenyl)-1-(2-fluoro-6-methoxy phenyl)-2-oxo-1,2-dihydropyridine-3-carboxamide

A solution of tert-butyl (1R,4R)-5-(4-fluoro-2-(1-(2-fluoro-6-methoxyphenyl)-2-oxo-1,2-dihydropyridine-3-carboxamido)phenyl)-2,5-diazabicyclo[2.2.1]heptane-2-carboxylate (200.98 umol, 1 eq.) in TFA (3 mL) was stirred for 15 mins to give a black brown solution. LCMS showed the starting material was consumed up and one major peak with desired mass was detected. The reaction was basified with con. Na₂CO₃ aq. to pH=10^{˜}11 and extracted with EA (10 mL×3). The combined organic layers were washed with brine, dried over Na₂SO₄, filtered and concentrated to dryness. The residue was purified by flash column (C18, eluted with 70% ACN in H₂O, 0.01% TFA). The collected flows were concentrated and lyophilized to afford Example 51 (38.5 mg, Yield: 42.34%) as an off-white solid.

LC-MS m/z: 453.2 (M+1)⁺.

¹H NMR (400 MHz, CD₃OD) δ 12.17 (d, J=10.5 Hz, 1H), 8.74 (dt, J=7.3, 2.0 Hz, 1H), 8.26 (ddd, J=11.1, 3.1, 1.9 Hz, 1H), 7.85 (dd, J=6.6, 2.2 Hz, 1H), 7.57 (td, J=8.6, 6.5 Hz, 1H), 7.26 (ddd, J=8.4, 5.5, 2.5 Hz, 1H), 7.16-7.07 (m, 1H), 7.04-6.94 (m, 1H), 6.87 (tdd, J=9.1, 3.1, 1.3 Hz, 1H), 6.76 (ddd, J=7.5, 6.6, 1.0 Hz, 1H), 4.38 (s, 1H), 3.94-3.90 (m, 1H), 3.88 (d, J=2.5 Hz, 3H), 3.70-3.56 (m, 1H), 3.47-3.38 (m, 2H), 3.20-3.13 (m, 1H), 2.38 (dd, J=16.1, 11.4 Hz, 1H), 1.91-1.82 (m, 1H).

| No. | Structure | Salt form | MS and ¹HNMR |
|---|---|---|---|
| Example 52 | 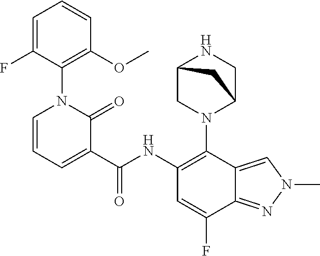 [SMILES: COc1cccc(F)c1-n1cccc(C(=O)Nc2cc(F)c3nn(C)cc3c2N2C[C@H]3C[C@@H]2CN3)c1=O] N-(4- ((1R,4R)-2,5- diazabicyclo[2.2.1]heptan-2-yl)-7- fluoro-2-methyl-2H-indazol-5-yl)- 1-(2-fluoro-6-methoxyphenyl)-2- oxo-1,2-dihydropyridine-3- carboxamide | TFA salt | LC-MS m/z: 507.2(M + 1)⁺. ¹H NMR (400 MHz, CD₃OD) δ 11.96 (s, 1H), 8.75 (dd, J = 7.3, 2.2 Hz, 1H), 8.50 (t, J = 3.0 Hz, 1H), 8.01 (ddd, J = 12.8, 5.7, 3.4 Hz, 1H), 7.87 (dd, J = 6.6, 2.1 Hz, 1H), 7.58 (td, J = 8.5, 6.5 Hz, 1H), 7.17-7.09 (m, 1H), 7.07-6.96 (m, 1H), 6.79 (t, J = 7.0 Hz, 1H), 4.49 (s, 1H), 4.24 (s, 1H), 4.23 (s, 3H), 3.89 (d, J = 6.8 Hz, 3H), 3.74-3.61 (m, 1H), 3.61- 3.52 (m, 2H), 3.30-3.18 (m, 1H), 2.49 (dd, J = 11.3, 8.8 Hz, 1H), 2.00 (d, J = 11.3 Hz, 1H). |
| Example 53 | 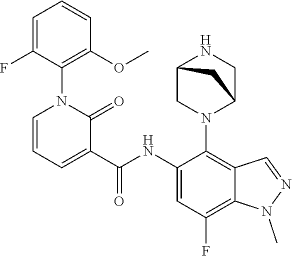 [SMILES: COc1cccc(F)c1-n1cccc(C(=O)Nc2cc(F)c3c(cnn3C)c2N2C[C@H]3C[C@@H]2CN3)c1=O] N-(4- ((1R,4R)-2,5- diazabicyclo[2.2.1]heptan-2-yl)-7- fluoro-1-methyl-1H-indazol-5-yl)- 1-(2-fluoro-6-methoxyphenyl)-2- oxo-1,2-dihydropyridine-3- carboxamide | TFA salt | LC-MS m/z: 507.2(M + 1)⁺. ¹H NMR (400 MHz, CD₃OD) δ 11.98 (d, J = 5.9 Hz, 1H), 8.75 (dd, J = 7.3, 2.1 Hz, 1H), 8.19 (dd, J = 5.4, 2.3 Hz, 1H), 8.15 (dt, J = 13.5, 3.4 Hz, 1H), 7.87 (dd, J = 6.6, 2.1 Hz, 1H), 7.58 (td, J = 8.6, 6.5 Hz, 1H), 7.17- 7.08 (m, 1H), 7.06-6.96 (m, 1H), 6.79 (t, J = 7.0 Hz, 1H), 4.50 (s, 1H), 4.31 (s, 1H), 4.22 (d, J = 1.2 Hz, 3H), 3.89 (d, J = 7.1 Hz, 3H), 3.73-3.63 (m, 1H), 3.67-3.56 (m, 2H), 3.27 (dd, J = 5.4, 2.1 Hz, 1H), 2.53 (t, J = 10.5 Hz, 1H), 2.01 (d, J = 11.5 Hz, 1H). |
| Example 54 | 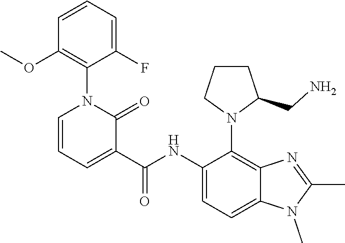 (S)-N-(4-(2- (aminomethyl)pyrrolidin-1-yl)-1,2- dimethyl-1H-benzo[d]imidazol-5- yl)-1-(2-fluoro-6-methoxyphenyl)- 2-oxo-1,2-dihydropyridine-3- carboxamide | TFA salt | LC-MS m/z: 505.5(M + 1)⁺ ¹H NMR (400 MHz, CD₃OD) δ 8.23 (d, J = 9.9 Hz, 1H), 7.99 (d, J = 8.7 Hz, 1H), 7.71 (tt, J = 8.6, 6.2 Hz, 1H), 7.39-7.34 (m, 1H), 7.34- 7.28 (m, 3H), 4.66 (td, J = 7.0, 3.8 Hz, 2H), 3.79 (s, 3H), 3.41-3.34 (m, 4H), 3.24 (dt, J = 8.9, 7.4 Hz, 1H), 2.76 (qd, J = 12.6, 5.7 Hz, 2H), 2.62 (s, 3H), 2.34 (dq, J = 14.5, 7.5 Hz, 1H), 1.98 (q, J = 5.8, 5.2 Hz, 1H), 1.74 (dq, J = 14.0, 7.6, 7.1 Hz, 1H), 1.63 (ddd, J = 14.6, 12.4, 6.4 Hz, 1H). |

### Example 55: 2-((2-((1R,4R)-2,5-diazabicyclo[2.2.1]heptan-2-yl)-5-fluorophenyl)carbamoyl)-6-(2-fluoro-6-methoxyphenyl)pyridine 1-oxide

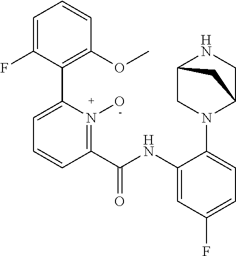

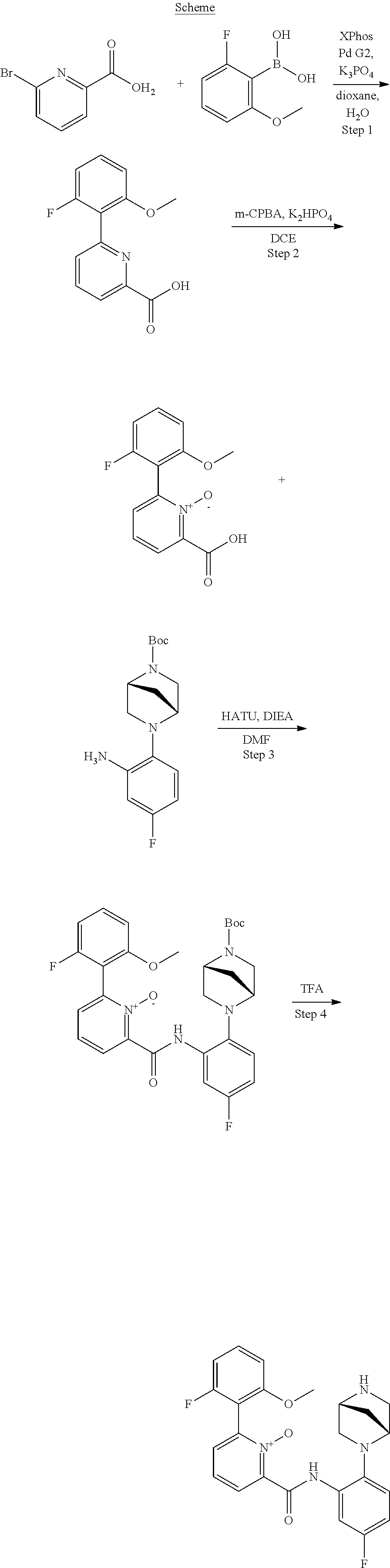

### Step 1: 6-(2-fluoro-6-methoxyphenyl)picolinic acid

To a solution of 6-bromopicolinic acid (1 g, 4.95 mmol, 1 eq.) and (2-fluoro-6-methoxyphenyl)boronic acid (0.84 g, 4.95 mmol, 1 eq.) in dioxane (15 mL) and H₂O (3 mL) was added K₃PO₄ (2.1 g, 9.9 mmol, 2 eq.) and XPhos Pd G2 (0.19 g, 0.25 mmol, 0.05 eq.) under N₂. The reaction was warmed to 80° C. and stirred for 2 hours to give a yellow suspension. LCMS showed most of the starting material was consumed and one major peak with desired mass was detected. The reaction was acidified with 1 N HCl to pH=3-4 and extracted with EA (20 mL×3). The combined organic layers were washed with brine, dried over Na₂SO₄, filtered and concentrated to dryness. The afford product was triturated with ACN and filtered. The filtrate was concentrated and lyophilized to afford 6-(2-fluoro-6-methoxyphenyl)picolinic acid (1.09 g, Yield: 89.34%) as an off-white solid.

LC-MS m/z: 246.2 (M−1)⁻.

### Step 2: 2-carboxy-6-(2-fluoro-6-methoxyphenyl)pyridine 1-oxide

To a solution of 6-(2-fluoro-6-methoxyphenyl)picolinic acid (1 g, 4.04 mmol, 1 eq.) in DCE (80 mL) was added K₂HPO₄ (3.52 g, 20.22 mmol, 5 eq.) and m-CPBA (4.11 g, 20.22 mmol, 5 eq.). The reaction was warmed to 70° C. and stirred for 5 hours to give a light yellow suspension. LCMS showed about half of the starting material was remained and one major peak with desired mass was detected. The reaction was stirred for another 5 hours at 70° C. to give a light yellow suspension. LCMS showed most of the starting material was consumed and one major peak with desired mass was detected. The reaction was diluted with H₂O and extracted with IPA/DCM=1/3 (30 mL×3). The combined organic layers were washed with brine, dried over Na₂SO₄, filtered and concentrated to afford 2-carboxy-6-(2-fluoro-6-methoxyphenyl) pyridine 1-oxide (1.2 g) as brown oil.

LC-MS m/z: 264.2 (M+1)⁺.

### Step 3: 2-((2-((1R,4R)-5-(tert-butoxycarbonyl)-2,5-diazabicyclo[2.2.1]heptan-2-yl)-5-fluorophenyl)carbamoyl)-6-(2-fluoro-6-methoxyphenyl)pyridine 1-oxide

To a solution of 2-carboxy-6-(2-fluoro-6-methoxyphenyl)pyridine 1-oxide (256.91 mg, 976.02 umol, 3 eq.) and tert-butyl (1R,4R)-5-(2-amino-4-fluorophenyl)-2,5-diazabicyclo [2.2.1]heptane-2-carboxylate (100 mg, 325.34 umol, 1 eq.) in DMF (3 mL) was added HATU (371.12 mg, 976.02 umol, 3 eq.) and DIEA (210.25 mg, 1.63 mmol, 5 eq.). The reaction was stirred for 1 hour at 26° C. to give a black brown solution. LCMS showed the starting material was consumed up and one major peak with desired mass was detected. The reaction was treated with H₂O and acidified with 1N HCl to pH=4^{˜}5. The mixture was extracted with EA (10 mL×3). The combined organic layers were washed with brine, dried over Na₂SO₄, filtered and concentrated to afford 2-((2-((1R,4R)-5-(tert-butoxycarbonyl)-2,5-diazabicyclo[2.2.1]heptan-2-yl)-5-fluorophenyl)carbamoyl)-6-(2-fluoro-6-methoxyphenyl)pyridine 1-oxide (450 mg, crude) as brown oil.

LC-MS m/z: 553.3 (M+1)⁺.

### Step 4: 2-((2-((1R,4R)-2,5-diazabicyclo[2.2.1]heptan-2-yl)-5-fluorophenyl)carbamoyl)-6-(2-fluoro-6-methoxyphenyl)pyridine 1-oxide

A solution of 2-((2-((1R,4R)-5-(tert-butoxycarbonyl)-2,5-diazabicyclo[2.2.1]heptan-2-yl)-5-fluorophenyl)carbamoyl)-6-(2-fluoro-6-methoxyphenyl)pyridine 1-oxide (325.34 umol, 1 eq.) in TFA (5 mL) was stirred for 30 mins at 26° C. to give a black brown solution. LCMS showed the starting material was consumed up and one major peak with desired mass was detected. The reaction was diluted with H₂O and basified with con. Na₂CO₃ aq. to pH=10-11. The mixture was extracted with EA (30 mL×3). The combined organic layers were washed with brine, dried over Na₂SO₄, filtered and concentrated to dryness. The residue was purified by flash column (C₁₈, eluted with 70% ACN in H₂O containing TFA). The collected flows were concentrated and lyophilized to afford Example 55 (104.4 mg, Yield: 70.92%) as an off-white foam.

LC-MS m/z: 453.2 (M+1)⁺.

¹H NMR (400 MHz, d₆-DMSO) δ 13.81 (s, 1H), 8.74 (s, 2H), 8.47 (ddd, R=8.0, 2.2, 0.8 Hz, 1H), 8.18 (ddd, J=11.2, 8.4, 3.1 Hz, 1H), 7.93-7.83 (i, 1H), 7.86-7.70 (m, 1H), 7.62-7.47 (m, 1H), 7.36 (dd, J=8.9, 5.6 Hz, 1H), 7.11-6.94 (m, 3H), 4.36 (s, 1H), 3.96 (d, J=4.4 Hz, 1H), 3.79 (d, J=1.3 Hz, 3H), 3.48 (t, =10.1 Hz, 1H), 3.04 (d, J=8.9 Hz, 1H), 2.14-2.03 (m, 1H), 1.74 (t, J=10.5 Hz, 1H).

| No. | Structure | Salt form | MS and ¹HNMR |
|---|---|---|---|
| Example 56 | 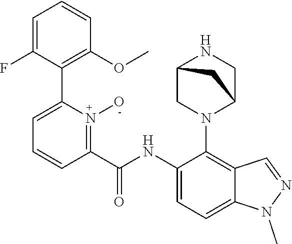 2-((4- ((1R,4R)-2,5- diazabicyclo[2.2.1]heptan-2-yl)- 1-methyl-1H-indazol-5- yl)carbamoyl)-6-(2-fluoro-6- methoxyphenyl)pyridine 1-oxide | TFA salt | LC-MS m/z: 489.2 (M + 1)⁺. ¹H NMR (400 MHz, CD₃OD) δ 8.63- 8.56 (m, 1H), 8.25-8.15 (m, 2H), 7.83-7.73 (m, 2H), 7.63-7.52 (m, 1H), 7.05 (dd, J = 10.8, 8.5 Hz, 1H), 6.93 (q, J = 8.4 Hz, 1H), 4.50 (s, 1H), 4.38 (s, 1H), 4.23 (d, J = 1.3 Hz, 3H), 3.85 (d, J = 8.5 Hz, 3H), 3.78-3.55 (m, 4H), 2.53 (t, J = 10.5 Hz, 1H), 2.03 (d, J = 10.9 Hz, 1H). |
| Example 57 | 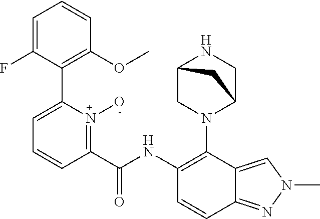 2-((4- ((1R,4R)-2,5- diazabicyclo[2.2.1]heptan-2-yl)- 2-methyl-2H-indazol-5- yl)carbamoyl)-6-(2-fluoro-6- methoxyphenyl)pyridine 1-oxide | TFA salt | LC-MS m/z: 489.2 (M + 1)⁺. ¹H NMR (400 MHz, CD₃OD) δ 8.60- 8.52 (m, 2H), 8.05 (dd, J = 12.8, 2.5 Hz, 1H), 7.76-7.70 (m, 2H), 7.55 (td, J = 8.5, 6.7 Hz, 1H), 7.03 (dd, J = 8.5, 3.0 Hz, 1H), 6.91 (td, J = 8.6, 2.7 Hz, 1H), 4.23 (s, 3H), 4.18 (d, J = 6.7 Hz, 1H), 3.84 (m, 4H), 3.64 (m, 1H), 3.58 (dd, J = 9.3, 6.1 Hz, 1H), 3.21 (d, J = 11.3 Hz, 1H), 2.91 (d, J = 9.9 Hz, 1H), 2.27-2.13 (m, 1H), 1.92-1.82 (m, 1H). |
| Example 58 | 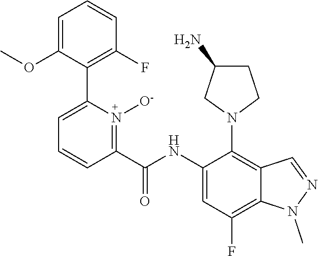 2-((4- ((S)-3-aminopyrrolidin-1-yl)-7- fluoro-1-methyl-1H-indazol-5- yl)carbamoyl)-6-(2-fluoro-6- methoxyphenyl)pyridine 1-oxide | HCl Salt | LC-MS m/z: 495.2 (M + 1)⁺. ¹H NMR (400 MHz, CD₃OD) δ 8.64 (ddd, J = 7.8, 5.3, 2.3 Hz, 1H), 8.46 (dd, J = 13.6, 9.0 Hz, 1H), 8.22 (dd, J = 2.4, 1.3 Hz, 1H), 7.89-7.75 (m, 2H), 7.62 (tdd, J = 8.5, 6.8, 2.7 Hz, 1H), 7.14-7.05 (m, 1H), 7.03- 6.93 (m, 1H), 4.23 (d, J = 1.1 Hz, 3H), 3.95 (s, 1H), 3.85 (d, J = 6.4 Hz, 3H), 3.61 (dt, J = 10.8, 6.0 Hz, 1H), 3.48-3.33 (m, 2H), 3.26 (d, J = 11.7 Hz, 1H), 2.59 (ddd, J = 12.0, 7.2, 4.1 Hz, 1H), 1.86 (td, J = 9.5, 8.8, 4.7 Hz, 1H). |
| Example 59 | 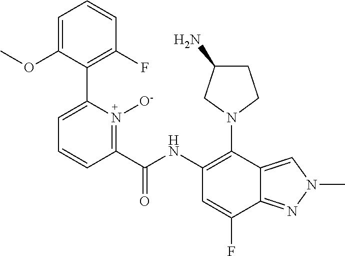 2-((4- ((S)-3-aminopyrrolidin-1-yl)-7- fluoro-2-methyl-2H-indazol-5- yl)carbamoyl)-6-(2-fluoro-6- methoxyphenyl)pyridine 1-oxide | HCl Salt | LC-MS m/z: 495.2 (M + 1)⁺. ¹H NMR (400 MHz, CD₃OD) δ 8.63 (ddd, J = 7.7, 5.2, 2.3 Hz, 1H), 8.56 (dd, J = 2.9, 1.4 Hz, 1H), 8.34 (dd, J = 12.9, 8.8 Hz, 1H), 7.89-7.75 (m, 2H), 7.63 (tdd, J = 8.5, 6.8, 2.7 Hz, 1H), 7.14-7.06 (m, 1H), 7.04- 6.93 (m, 1H), 4.24 (s, 3H), 3.94 (s, 1H), 3.86 (d, J = 5.8 Hz, 3H), 3.54 (dt, J = 10.9, 5.5 Hz, 1H), 3.42-3.32 (m, 2H), 3.22 (d, J = 10.7 Hz, 1H), 2.65-2.50 (m, 1H), 1.87 (ddd, J = 14.2, 8.3, 2.9 Hz, 1H). |
| Example 60 | 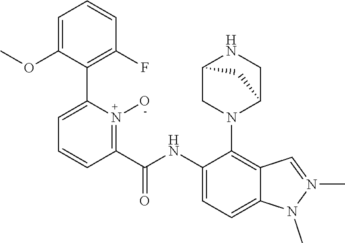 2-((4- ((1S,4S)-2,5- diazabicyclo[2.2.1]heptan-2-yl)- 1,2-dimethyl-1H- benzo[d]imidazol-5- yl)carbamoyl)-6-(2-fluoro-6- methoxyphenyl)pyridine 1-oxide Molecular Weight: 502.55 | HCl Salt | LC-MS m/z: 503.5 (M + 1)⁺. ¹H NMR (400 MHz, CD₃OD) δ 8.66- 8.54 (m, 1H), 8.48 (dd, J = 8.8, 2.4 Hz, 1H), 7.80-7.68 (m, 2H), 7.58 (dt, J = 15.2, 7.6 Hz, 1H), 7.40 (d, J = 8.9 Hz, 1H), 7.06 (d, J = 8.5 Hz, 1H), 6.93 (t, J = 8.8 Hz, 1H), 4.49 (s, 1H), 4.29 (d, J = 50.1 Hz, 1H), 4.15 (s, 1H), 3.87 (d, J = 3.5 Hz, 3H), 3.83 (s, 3H), 3.66 (d, J = 17.3 Hz, 2H), 3.15 (d, J = 1.7 Hz, 1H), 2.69 (s, 3H), 2.61 (d, J = 10.3 Hz, 1H), 1.99 (s, 1H). |
| Example 61 | 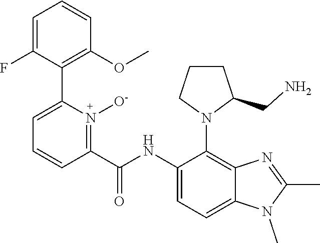 2-((4-((S)-2- (aminomethyl)pyrrolidin-1-yl)- 1,2-dimethyl-1H- benzo[d]imidazol-5- yl)carbamoyl)-6-(2-fluoro-6- methoxyphenyl)pyridine 1-oxide | Free base | LC-MS m/z: 505.2 (M + 1)⁺. ¹H NMR (400 MHz, CD₃OD) δ 8.59 (dd, J = 10.8, 5.6 Hz, 1H), 8.53 (dd, J = 16.3, 8.9 Hz, 1H), 7.81-7.70 (m, 2H), 7.57 (tt, J = 8.4, 6.5 Hz, 1H), 7.36 (t, J = 8.5 Hz, 1H), 7.05 (t, J = 8.2 Hz, 1H), 6.98-6.89 (m, 1H), 4.53 (d, J = 3.3 Hz, 1H), 3.86 (d, J = 2.7 Hz, 3H), 3.80 (s, 3H), 3.46-3.37 (m, 1H), 3.26 (dd, J = 17.6, 8.6 Hz, 1H), 2.72-2.60 (m, 4H), 2.56 (dt, J = 13.2, 4.3 Hz, 1H), 2.47-2.32 (m, 1H), 2.07-1.89 (m, 2H), 1.87- 1.77 (m, 1H). |
| Example 62 | 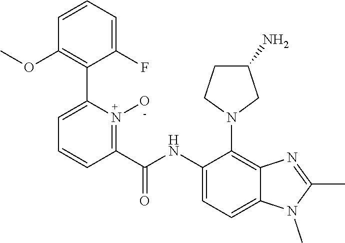 2-((4-((S)-3-aminopyrrolidin-1- yl)-1,2-dimethyl-1H- benzo[d]imidazol-5- yl)carbamoyl)-6-(2-fluoro-6- methoxyphenyl)pyridine 1-oxide | Free base | LC-MS m/z: 491.2 (M + 1)⁺. ¹H NMR (400 MHz, CD₃OD) δ 8.65 (ddd, J = 7.9, 5.8, 3.1 Hz, 2H), 7.88- 7.77 (m, 2H), 7.63 (tdd, J = 8.5, 6.8, 3.2 Hz, 1H), 7.55 (dd, J = 9.0, 1.7 Hz, 1H), 7.11 (t, J = 8.2 Hz, 1H), 6.99 (td, J = 8.8, 5.5 Hz, 1H), 3.97 (s, 1H), 3.92-3.82 (m, 7H), 3.62- 3.51 (m, 1H), 3.50-3.40 (m, 1H), 3.29 (d, J = 10.8 Hz, 1H), 2.66-2.53 (m, 1H), 1.98 (dd, J = 14.8, 7.2 Hz, 1H). |
| Example 63 | 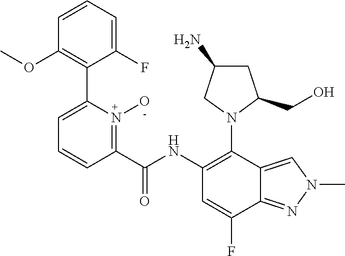 2-((4- ((2S,4S)-4-amino-2- (hydroxymethyl)pyrrolidin-1-yl)- 7-fluoro-2-methyl-2H-indazol-5- yl)carbamoyl)-6-(2-fluoro-6- methoxyphenyl)pyridine 1-oxide | TFA salt | LC-MS m/z: 525.3 (M + 1)⁺ ¹H NMR (400 MHz, CD₃OD) δ 8.6 (dd, J = 7.6, 2.8 Hz, 2H), 8.5-8.3 (m, 1H), 7.8-7.7 (m, 2H), 7.6 (tdd, J = 8.4, 6.8, 1.2 Hz,1H), 7.0 (dd, J = 8.4, 6.4 Hz, 1H), 7.0-6.9 (m, 1H), 4.2 (s, 3H), 4.0-3.7 (m, 5H), 3.7- 3.5 (m, 1H), 3.3-3.1 (m, 3H), 2.8 (ddd, J = 14.4, 10.4, 6.4 Hz, 1H), 2.0 (dd, J = 21.2, 16.4 Hz, 1H). |

### Example 64: N-(2-((1S,4S)-2,5-diazabicyclo[2.2.1]heptan-2-yl)-5-fluorophenyl)-2-(2-fluoro-6-methoxyphenyl)thiazole-4-carboxamide

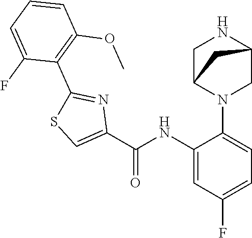 [SMILES: COc1cccc(F)c1-c1nc(C(=O)Nc2cc(F)ccc2N2C[C@@H]3C[C@H]2CN3)cs1]

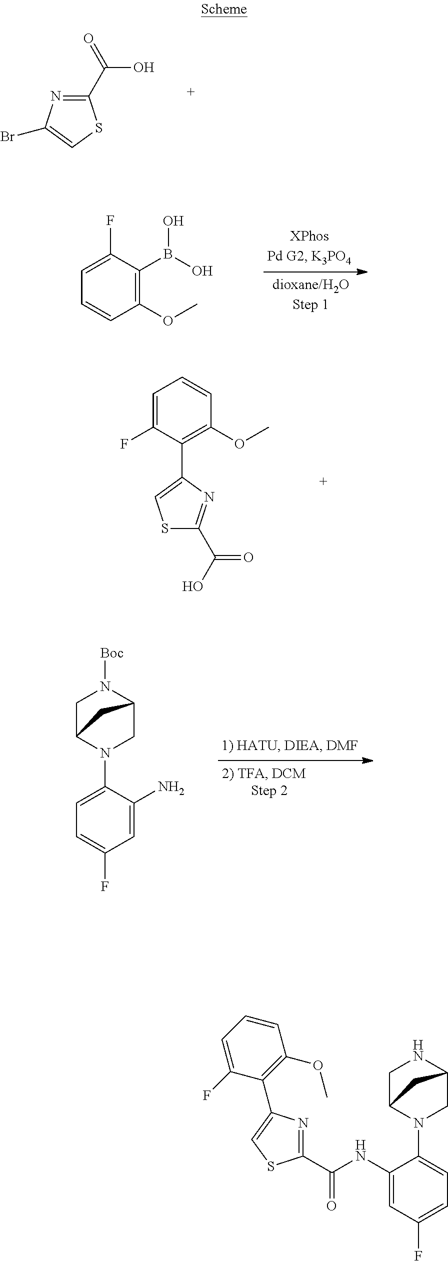 [SMILES: O=C(O)c1nc(Br)cs1 ; COc1cccc(F)c1B(O)O ; COc1cccc(F)c1-c1csc(C(=O)O)n1 ; CC(C)(C)OC(=O)N1C[C@@H]2C[C@H]1CN2c1ccc(F)cc1N ; COc1cccc(F)c1-c1csc(C(=O)Nc2cc(F)ccc2N2C[C@@H]3C[C@H]2CN3)n1]

### Step 1: 2-(2-fluoro-6-methoxyphenyl)thiazole-4-carboxylic acid

To a solution of 4-bromothiazole-2-carboxylic acid (1 g, 4.81 mmol, 1 eq.) and (2-fluoro-6-methoxyphenyl)boronic acid (0.98 g, 5.77 mmol, 1.2 eq.) in dioxane (15 mL) and H₂O (3 mL) was added K₃PO₄ (2.04 g, 9.61 mmol, 2 eq.) and Xphos Pd G2 (113.47 mg, 0.14 mmol, 0.03 eq.) under N₂. The reaction was warmed to 80° C. in microwave and stirred for 1.5 hours to give an off-white suspension. LCMS showed one peak with desired mass was detected and the starting material was consumed up. The reaction was diluted with DCM/MeOH=10/1 and filtered through a pad of Celite. The solution was concentrated to dryness. The residue was triturated with EA, filtered and dried in air to afford 2-(2-fluoro-6-methoxyphenyl)thiazole-4-carboxylic acid (0.85 g, 69.67%) as a gray solid.

LC-MS m/z: 252.1 (M−1)⁻.

### Step 2: N-(2-((1S,4S)-2,5-diazabicyclo[2.2.1]heptan-2-yl)-5-fluorophenyl)-4-(2-fluoro-6-methoxyphenyl)thiazole-2-carboxamide

To a solution of 2-(2-fluoro-6-methoxyphenyl)thiazole-4-carboxylic acid (404.54 mg, 1.60 mmol, 1 eq.) in DMF (5 mL) was added HATU (911.10 mg, 2.40 mmol, 1.5 eq.) and DIEA (412.93 mg, 3.19 mmol, 2 eq.) under N₂. After stirring for 5 mins, (2-fluoro-6-methoxyphenyl)boronic acid (491 mg, 1.60 mmol, 1 eq.) was added to the reaction. The resulting mixture was stirred for 0.5 hour to give a black brown solution. LCMS showed two peaks with desired mass was detected. The reaction was diluted with H₂O and extracted with DCM (10 mL×3). The combined organic layers were washed with brine, dried over Na₂SO₄, filtered and concentrated to dryness. The afford product was dissolved in TFA (3 mL) and stirred for 3 hours at 45° C. to give a black brow solution. LCMS showed two peaks with desired mass was detected. The reaction was concentrated to remove most of the solvent and purified by flash column (C₁₈, eluted with 90% ACN in H₂O containing TFA). The collected flows were concentrated and lyophilized to afford Example 64 (9.9 mg) as a gray solid.

LC-MS m/z: 443.2 (M+1)⁺.

¹H NMR (400 MHz, de-DMSO): δ 10.05 (s, 1H), 8.87 (s, 1H), 8.78 (s, 1H), 8.71 (s, 1H), 8.11 (dd, J=10.6, 3.0 Hz, 1H), 7.64-7.54 (m, 1H), 7.39 (dd, J=9.0, 5.6 Hz, 1H), 7.15 (d, J=8.5 Hz, 1H), 7.11-6.98 (m, 2H), 4.43 (s, 1H), 4.01 (s, 1H), 3.97 (s, 3H), 3.52 (d, J=11.1 Hz, 1H), 3.10 (s, 1H), 2.21 (d, J=11.2 Hz, 1H), 1.88 (d, J=10.8 Hz, 1H).

### Example 65: N-(2-((1R,4R)-2,5-diazabicyclo[2.2.1]heptan-2-yl)-5-fluorophenyl)-2-(2-fluoro-6-methoxyphenyl)thiazole-4-carboxamide

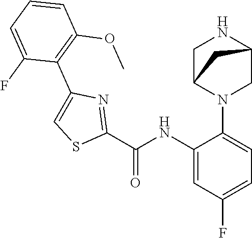 [SMILES: COc1cccc(F)c1-c1csc(C(=O)Nc2cc(F)ccc2N2C[C@@H]3C[C@H]2CN3)n1]

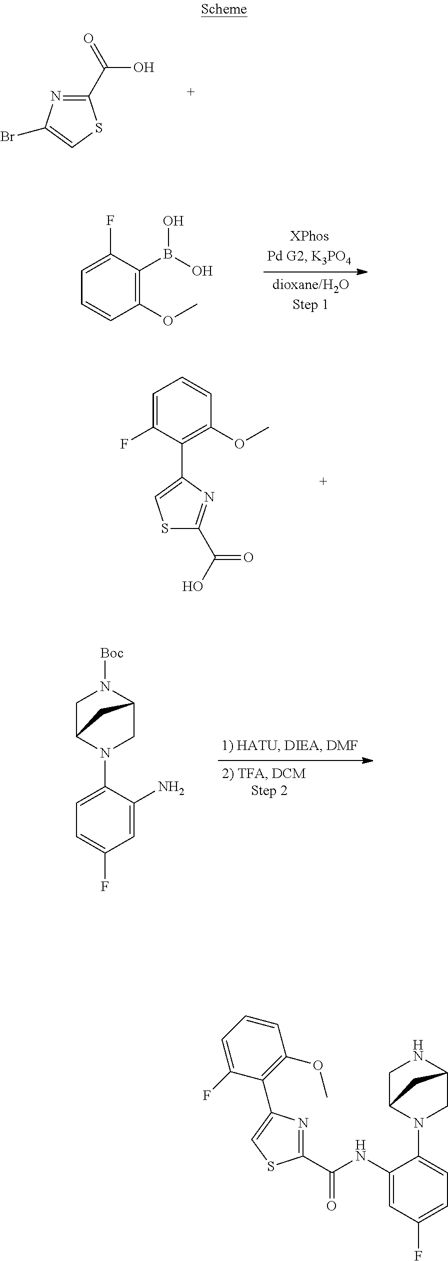 [SMILES: O=C(O)c1nc(Br)cs1 ; COc1cccc(F)c1B(O)O ; COc1cccc(F)c1-c1csc(C(=O)O)n1 ; CC(C)(C)OC(=O)N1C[C@@H]2C[C@H]1CN2c1ccc(F)cc1N ; COc1cccc(F)c1-c1csc(C(=O)Nc2cc(F)ccc2N2C[C@@H]3C[C@H]2CN3)n1]

### Step 1: 4-(2-fluoro-6-methoxyphenyl)thiazole-2-carboxylic acid

To a solution of 4-bromothiazole-2-carboxylic acid (1 g, 4.81 mmol, 1 eq.) and (2-fluoro-6-methoxyphenyl)boronic acid (0.98 g, 5.77 mmol, 1.2 eq.) in dioxane (15 mL) and H₂O (3 mL) was added K₃PO₄ (2.04 g, 9.61 mmol, 2 eq.) and Xphos Pd G2 (113.47 mg, 0.14 mmol, 0.03 eq.) under N₂. The reaction was warmed to 80° C. in microwave and stirred for 1.5 hours to give an off-white suspension. LCMS showed one peak with desired mass was detected and the starting material was consumed up. The reaction was diluted with DCM/MeOH=10/1 and filtered through a pad of Celite. The solution was concentrated to dryness. The residue was triturated with EA, filtered and dried in air to afford 4-(2-fluoro-6-methoxyphenyl)thiazole-2-carboxylic acid (0.57 g, Yield: 46.72%) as an off-white solid.

LC-MS m/z: 252.1 (M−1)⁻.

### Step 2: N-(2-((1S,4S)-2,5-diazabicyclo[2.2.1]heptan-2-yl)-5-fluorophenyl)-4-(2-fluoro-6-methoxyphenyl)thiazole-2-carboxamide

To a solution of 4-(2-fluoro-6-methoxyphenyl)thiazole-2-carboxylic acid (247.18 mg, 0.98 mmol, 1 eq.) in DMF (3 mL) was added HATU (556.68 mg, 1.46 mmol, 1.5 eq.) and DIEA (252.30 mg, 1.95 mmol, 2 eq.) under N₂. After stirring for 5 mins, tert-butyl (1R,4R)-5-(2-amino-4-fluorophenyl)-2,5-diazabicyclo[2.2.1]heptane-2-carboxylate (300 mg, 0.98 mmol, 1 eq.) was added to the reaction. The resulting mixture was stirred for 0.5 hour to give a black brown solution. LCMS showed one major peak with desired mass was detected. The reaction was diluted with H₂O and extracted with DCM (10 mL×3). The combined organic layers were washed with brine, dried over Na₂SO₄, filtered and concentrated to dryness. The afford product was dissolved in TFA (3 mL) and stirred for 16 hours to give a black brow solution. LCMS showed one major peak with desired mass was detected. The reaction was concentrated to remove most of the solvent and purified by flash column (C₁₈, eluted with 90% ACN in H₂O containing TFA). The collected flows were concentrated and lyophilized to afford Example 65 (141.5 mg, Yield: 32.76%) as an off-white solid.

LC-MS m/z: 443.2 (M+1)⁺.

¹H NMR (400 MHz, de-DMSO): δ 10.13 (s, 1H), 8.99 (s, 1H), 8.78 (s, 1H), 8.22 (s, 1H), 7.82 (dd, J=10.3, 3.0 Hz, 1H), 7.55-7.45 (m, 1H), 7.30 (dd, J=9.3, 5.4 Hz, 1H), 7.05 (t, J=7.6 Hz, 2H), 6.97 (t, J=8.9 Hz, 1H), 4.41 (d, J=3.3 Hz, 1H), 4.08 (s, 1H), 3.81 (s, 3H), 3.55 (d, J=11.0 Hz, 1H), 3.30 (d, J=11.4 Hz, 1H), 3.12 (s, 1H), 2.09 (d, J=11.0 Hz, 1H), 1.84 (d, J=10.9 Hz, 1H).

Example 66: N-(2-((1R,4R)-2,5-diazabicyclo[2.2.1]heptan-2-yl)-5-fluorophenyl)-2-(2-fluoro-6-met(S)—N-(4-(3-aminopyrrolidin-1-yl)-1,2-dimethyl-1H-benzo[d]imidazol-5-yl)-1-(2-fluoro-6-methoxyphenyl)-6-oxo-1,6-dihydropyridazine-3-carboxamide

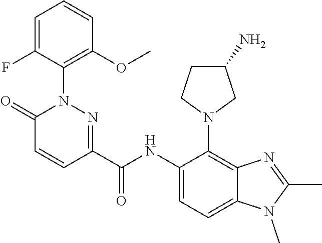 [SMILES: COc1cccc(F)c1-n1nc(C(=O)Nc2ccc3c(nc(C)n3C)c2N2CC[C@H](N)C2)ccc1=O]

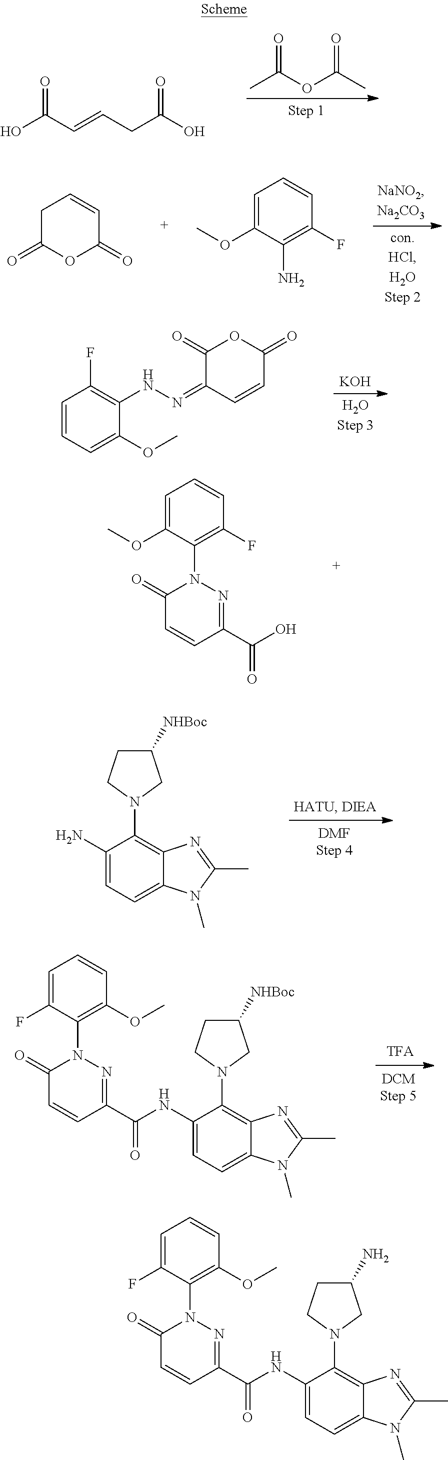

### Step 1: 2H-pyran-2,6(3H)-dione

A solution of (E)-pent-2-enedioic acid (3 g, 23.06 mmol, 1 eq.) in acetic anhydride (9.42 g, 92.24 mmol, 4 eq.) was stirred for 1 hour at 150° C. to give a black brown solution. No monitored. The reaction was concentrated to afford 2H-pyran-2,6(3H)-dione (2.6 g, Yield: 100.78%) as a black solid.

### Step 2: (Z)-3-(2-(2-fluoro-6-methoxyphenyl)hydrazineylidene)-2H-pyran-2,6(3H)-dione

To a solution of 2-fluoro-6-methoxyaniline (1.5 g, 10.63 mmol, 1 eq.) in H₂O (40 mL) and 12 N HCl (12 mL) was added NaNO₂ (0.95 g, 13.82 mmol, 1.3 eq.) in portions at 0° C. After stirring for 1 hour at 0° C., a solution of 2H-pyran-2,6(3H)-dione (1.19 g, 10.63 mmol, 1 eq.) and Na₂CO₃ (0.56 g, 5.31 mmol, 0.5 eq.) in H₂O (40 mL) was added to the reaction dropwise at 0° C. The reaction was stirred for 16 hours to give a brown suspension. LCMS showed the reaction was messy and one peak with desired mass was detected. The reaction was filtered and the filter cake was washed with H₂O and dried by oil pump to afford (Z)-3-(2-(2-fluoro-6-methoxyphenyl)hydrazineylidene)-2H-pyran-2,6(3H)-dione (1 g, Yield: 35.59%) as a brown solid.

LC-MS m/z: 265.1 (M+1)⁺.

### Step 3: 1-(2-fluoro-6-methoxyphenyl)-6-oxo-1,6-dihydropyridazine-3-carboxylic acid

To a solution of (Z)-3-(2-(2-fluoro-6-methoxyphenyl)hydrazineylidene)-2H-pyran-2,6(3H)-dione (1 g, 3.78 mmol, 1 eq.) in H₂O (30 mL) was added KOH (1.06 g, 18.92 mmol, 5 eq.). The reaction was stirred for 3 hours at 100° C. to give a black brown solution. LCMS showed the starting material was consumed up and one new peak with desired mass was detected. The reaction was acidified with 1 N HCl to pH=2^{˜}3 and extracted with EA (20 mL×3). The combined organic layers were washed with brine, dried over Na₂SO₄, filtered and concentrated to dryness. The residue was purified by flash column (C₁₈, eluted with 62% ACN in H₂O, containing 0.1% HCOOH). The collected flows were acidified with 1 N HCl to pH=2^{˜}3 and extracted with EA (20 mL×3). The combined organic layers were washed with brine, dried over Na₂SO₄, filtered and concentrated to dryness. The afford product was lyophilized to afford 1-(2-fluoro-6-methoxyphenyl)-6-oxo-1,6-dihydropyridazine-3-carboxylic acid (103 mg, Yield: 10.3%) as a light brown solid. LC-MS m/z: 263.1 (M−1)⁻.

### Step 4: tert-butyl (S)-(1-(5-(1-(2-fluoro-6-methoxyphenyl)-6-oxo-1,6-dihydropyridazine-3-carboxamido)-1,2-dimethyl-1H-benzo[d]imidazol-4-yl)pyrrolidin-3-yl)carbamate

To a solution of 1-(2-fluoro-6-methoxyphenyl)-6-oxo-1,6-dihydropyridazine-3-carboxylic acid (70 mg, 264.94 umol, 1 eq.) and tert-butyl (S)-(1-(5-amino-1,2-dimethyl-1H-benzo[d]imidazol-4-yl)pyrrolidin-3-yl)carbamate (100.67 mg, 291.43 umol, 1.1 eq.) in DMF (2 mL) was added HATU (151.11 mg, 397.41 umol, 1.5 eq.) and DIEA (102.73 mg, 794.82 umol, 3 eq.). The reaction was stirred for 16 hours at 18° C. to give a black brown solution. LCMS showed the starting material was consumed up and one peak with desired mass was detected. The reaction was treated with H₂O and extracted with EA (10 mL×3). The combined organic layers were washed with brine, dried over Na₂SO₄, filtered and concentrated to afford tert-butyl (S)-(1-(5-(1-(2-fluoro-6-methoxyphenyl)-6-oxo-1,6-dihydropyridazine-3-carboxamido)-1,2-dimethyl-1H-benzo[d]imidazol-4-yl)pyrrolidin-3-yl)carbamate (205 mg, Yield: 130.78%) as a light brown solid. LC-MS m/z: 592.3 (M+1)⁺.

### Step 5: (S)—N-(4-(3-aminopyrrolidin-1-yl)-1,2-dimethyl-1H-benzo[d]imidazol-5-yl)-1-(2-fluoro-6-methoxyphenyl)-6-oxo-1,6-dihydropyridazine-3-carboxamide

To a solution of tert-butyl (S)-(1-(5-(1-(2-fluoro-6-methoxyphenyl)-6-oxo-1,6-dihydropyridazine-3-carboxamido)-1,2-dimethyl-1H-benzo[d]imidazol-4-yl)pyrrolidin-3-yl)carbamate (264.94 umol, 1 eq.) in DCM (2 mL) was added TFA (2 mL). The reaction was stirred for 30 mins to give a black brown solution. LCMS showed the starting material was consumed up and one major peak with desired mass was detected. The reaction was basified with con. NaHCO₃ aq. to pH=9^{˜}10 and extracted with DCM/i-PrOH=3/1 (10 mL×3). The combined organic layer washed with brine, dried over MgSO₄, filtered and concentrated to dryness. The residue was purified by flash column (C₁₈, eluted with 48% ACN in H₂O, containing 0.1% HCOOH) and the flows were lyophilized to afford Example 66 (32.18 mg, Yield: 24.72%) as a light yellow solid.

LC-MS m/z: 492.2 (M+1)⁺.

¹H NMR (400 MHz, CD₃OD): δ 8.27 (dd, J=8.8, 3.4 Hz, 1H), 8.20 (d, J=9.8 Hz, 1H), 7.59 (td, J=8.6, 6.5 Hz, 1H), 7.37 (dd, J=8.8, 1.6 Hz, 1H), 7.27 (d, J=9.8 Hz, 1H), 7.15-7.08 (m, 1H), 7.02 (tt, J=8.5, 1.1 Hz, 1H), 3.89 (d, J=1.5 Hz, 3H), 3.79 (s, 3H), 3.65-3.37 (m, 3H), 3.35 (s, 1H), 3.28-3.11 (m, 1H), 2.62 (s, 3H), 2.13-1.93 (m, 2H).

### Example 67: (R)—N-(4-(3-aminopiperidin-1-yl)-1,2-dimethyl-1H-benzo[d]imidazol-5-yl)-1-(2,6-difluorophenyl)-6-oxo-1,6-dihydropyridazine-3-carboxamide

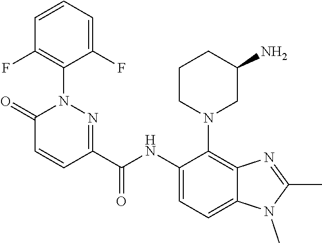 [SMILES: Cc1nc2c(N3CCC[C@@H](N)C3)c(NC(=O)c3ccc(=O)n(-c4c(F)cccc4F)n3)ccc2n1C]

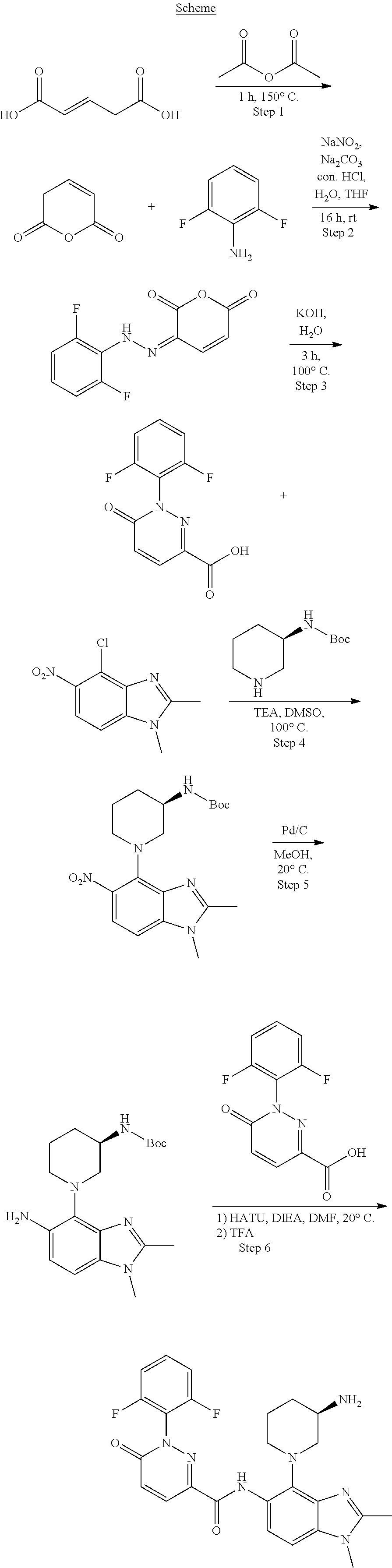

### Step 1: 2H-pyran-2,6(3H)-dione

A solution of (E)-pent-2-enedioic acid (15 g, 115.3 mmol, 1 eq.) in acetic anhydride (47.08 g, 461.19 mmol, 4 eq.) was stirred for 1 hour at 150° C. to give a black brown solution. The reaction was concentrated to afford 2H-pyran-2,6(3H)-dione (14.3 g, 110.7%) as a black solid. Step 2: (Z)-3-(2-(2,6-difluorophenyl)hydrazono)-2H-pyran-2,6(3H)-dione

To a solution of 2,6-difluoroaniline (22.33 g, 172.95 mmol, 1.5 eq.) in H₂O (300 mL) and 12 N HCl (80 mL) was added NaNO₂ (11.93 g, 172.95 mmol, 1.5 eq.) in portions at 0° C. to give a light brown solution. After stirring for 1 hour at 0° C., a solution of 2H-pyran-2,6(3H)-dione (115.3 mmol, 1 eq.) and Na₂CO₃ (7.33 g, 69.18 mmol, 0.6 eq.) in H₂O (100 mL) and THF (50 mL) was added to the reaction dropwise at 0° C. The reaction was stirred for 16 hours at rt to give a brown suspension. LCMS showed the starting material was consumed up and one peak with desired mass was detected. The reaction was filtered and the filter cake was washed with H₂O and dried by oil pump to afford (Z)-3-(2-(2,6-difluorophenyl)hydrazono)-2H-pyran-2,6(3H)-dione (23 g, Yield: 79.3%) as a brown solid.

LC-MS m/z: 253.1 (M+1)⁺

### Step 3: 1-(2,6-difluorophenyl)-6-oxo-1,6-dihydropyridazine-3-carboxylic acid

To a solution of (Z)-3-(2-(2,6-difluorophenyl)hydrazineylidene)-2H-pyran-2,6(3H)-dione (23 g, 91.21 mmol, 1 eq.) in H₂O (300 mL) was added KOH (10.23 g, 182.41 mmol, 2 eq.). The reaction was stirred for 3 hours at 100° C. to give a black brown solution. LCMS showed the starting material was consumed up and one new peak with desired mass was detected. The reaction was acidified with 1 N HCl to pH=2^{˜}3 and extracted with EA (20 mL×3). The combined organic layers were washed with brine, dried over Na₂SO₄, filtered and concentrated to dryness. The residue was purified by flash column (Silica column, eluted with 30% to 50% EA in PE) to afford 1-(2,6-difluorophenyl)-6-oxo-1,6-dihydropyridazine-3-carboxylic acid (5 g) as a light red solid. LC-MS m/z: 253.1 (M+1)⁺

### Step 4: tert-butyl (R)-(1-(1,2-dimethyl-5-nitro-1H-benzo[d]imidazol-4-yl)piperidin-3-yl) carbamate

To a solution of 4-chloro-1,2-dimethyl-5-nitro-1H-benzo[d]imidazole (300 mg, 1.33 mmol, 1.0 eq.) in DMSO (5 mL) was added TEA (269.09 mg, 2.66 mmol, 2.0 eq.) and tert-butyl (R)-piperidin-3-ylcarbamate (532.59 mg, 2.66 mmol, 2.0 eq.). The reaction was stirred at 100° C. for 2 h to give a yellow solution. LCMS showed the major peak with the desired mass and the material was almost consumed. The reaction was poured into water and filtered. The filter cake was dryness to give compound tert-butyl (R)-(1-(1,2-dimethyl-5-nitro-1H-benzo[d]imidazol-4-yl) piperidin-3-yl) carbamate (517.8 mg, Yield: 100%) as a yellow solid.

LC-MS m/z: 390.2 (M+1)⁺

### Step 5: tert-butyl (R)-(1-(5-amino-1,2-dimethyl-1H-benzo[d]imidazol-4-yl)piperidin-3-yl)carbamate

To a solution of tert-butyl (R)-(1-(1,2-dimethyl-5-nitro-1H-benzo[d]imidazol-4-yl)piperidin-3-yl)carbamate (517.82 mg, 1.33 mmol, 1.0 eq.) in MeOH (5 mL) was added Pd/C (26 mg). The reaction was stirred at 20° C. overnight to give a brown green suspension. LCMS showed the major peak with the desired mass and the material was almost consumed. The reaction was diluted with DCM:MeOH=10:1 and filtered through a pad of Celite. The filtrate was concentrated to dryness to afford compound tert-butyl (R)-(1-(5-amino-1,2-dimethyl-1H-benzo[d]imidazol-4-yl) piperidin-3-yl) carbamate (532 mg, Yield: 111%) as a brown solid.

LC-MS m/z: 360.23 (M+1)⁺

### Step 6: (R)—N-(4-(3-aminopiperidin-1-yl)-1,2-dimethyl-1H-benzo[d]imidazol-5-yl)-1-(2,6-difluorophenyl)-6-oxo-1,6-dihydropyridazine-3-carboxamide

To a solution of 1-(2,6-difluorophenyl)-6-oxo-1,6-dihydropyridazine-3-carboxylic acid (50 mg, 198.27 umol, 1.0 eq.) in DMF (1 mL) was added HATU (113.09 mg, 297.41 umol, 1.5 eq.), DIEA (0.065 mL) and stirred at 20° C. for 40 min, then the tert-butyl (R)-(1-(5-amino-1,2-dimethyl-1H-benzo[d]imidazol-4-yl)piperidin-3-yl) carbamate (71.27 mg, 198.27 umol, 1.0 eq.) was added. The reaction was stirred at 20° C. for overnight to give a brown suspension. LCMS showed one of the major peak with the desired mass and the material was almost consumed. The reaction was washed with water and extracted with DCM (3×), the organic phase was concentrated under reduced pressure to dryness and added TFA (1 mL) and stirred for 10 min. The mixture was adjusted pH7^{˜}8 by NaHCO₃ and extracted with DCM (3×), the organic phase was concentrated under reduced pressure to dryness. The crude product was purified by flash column (C₁₈, peak time=30% ACN in H₂O, COMBI FLASH NEXTAGEN 300) to give Example 67 (57.83 mg, Yield: 49%) as a yellow solid.

LC-MS m/z: 494.20 (M+1)⁺.

¹H NMR (400 MHz, CD₃OD) δ 8.40 (d, ==8.8 Hz, 1H), 8.24 (d, 8=9.8 Hz, 1H), 7.74 (n, J=8.6, 6.3 Hz, 1H), 7.43-7.31 (m, 4H), 3.79 (s, 3H), 3.60 (t, J=10.7 Hz, 1H), 3.37 (m, J=11.7, 2.5 Hz, 1H), 3.22-3.11 (m, 1H), 3.00-2.79 (m, 2H), 2.63 (s, 3H), 1.90-1.69 (m, 2H), 1.52 (m, J=23.9, 12.2, 3.7 Hz, 1H), 1.32 (m, J=16.3, 8.6 Hz, 1H).

| No. | Structure | Salt form | MS and 1HNMR |
|---|---|---|---|
| Example 68 | 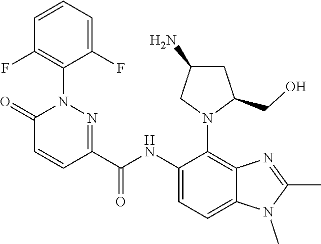 [SMILES: Cc1nc2c(N3C[C@@H](N)C[C@H]3CO)c(NC(=O)c3ccc(=O)n(-c4c(F)cccc4F)n3)ccc2n1C] N-(4-((2S,4S)-4-amino-2- (hydroxymethyl)amidazole-1-yl)- 1,2-dimethyl-1H- benzo[d]amidazole-5-yl)-1-(2,6- difluorophenyl)-6-oxo-1,6- dihydropyridazine-3-carboxamide | HCOOH salt | LC-MS m/z: 510.20(M + 1)⁺. ¹H NMR (400 MHz, CD₃OD) δ 8.43 (d, J = 8.9 Hz, 1H), 8.28 (d, J = 9.9 Hz, 1H), 7.79-7.67 (m, 1H), 7.45 (d, J = 8.9 Hz, 1H), 7.34 (t, J = 9.1 Hz, 3H), 4.11 (d, J = 8.8 Hz, 1H), 3.96 (m, J = 10.4, 3.6 Hz, 1H), 3.87 (t, J = 4.5 Hz, 1H), 3.81 (s, 3H), 3.21 (d, J = 9.2 Hz, 1H), 2.83 (s, 2H), 2.64 (s, 3H), 1.97 (d, J = 14.3 Hz, 1H), 1.88 (s, 1H). |
| Example 69 | 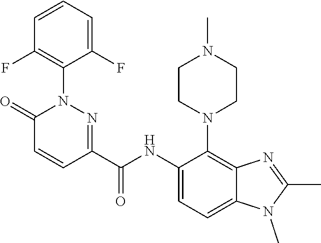 [SMILES: Cc1nc2c(N3CCN(C)CC3)c(NC(=O)c3ccc(=O)n(-c4c(F)cccc4F)n3)ccc2n1C] 1-(2,6-difluorophenyl)-N-(1,2- dimethyl-4-(4-methylpiperazin-1- yl)-1H-benzo[d]imidazol-5-yl)-6- oxo-1,6-dihydropyridazine-3- carboxamide | HCOOH salt | LC-MS m/z:494.2(M + 1)⁺. ¹H NMR (400 MHz, CD₃OD) δ 8.36 (d, J = 8.8 Hz, 1H), 8.30- 8.25 (m, 1H), 7.74 (m, J = 16.1, 6.8 Hz, 1H), 7.42 (t, J = 8.6 Hz, 3H), 7.35 (d, J = 9.9 Hz, 1H), 3.93 (s, 2H), 3.81 (d, J = 5.4 Hz, 3H), 3.53 (d, J = 19.6 Hz, 2H), 3.25-3.15 (m, 2H), 2.79 (d, J = 36.4 Hz, 2H), 2.73 (s, 3H), 2.63 (s, 3H). |
| Example 70 | 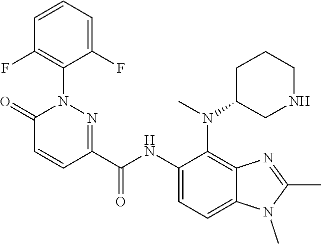 [SMILES: Cc1nc2c(N(C)[C@@H]3CCCNC3)c(NC(=O)c3ccc(=O)n(-c4c(F)cccc4F)n3)ccc2n1C] (R)-1- (2,6-difluorophenyl)-N-(1,2- dimethyl-4-(methyl(piperidin-3-yl) amino)-1H-benzo[d]imidazol-5-yl)- 6-oxo-1,6-dihydropyridazine-3- carboxamide | HCOOH salt | LC-MS m/z: 508.22(M + 1)⁺. ¹H NMR (400 MHz, CD₃OD) δ 8.44 (d, J = 8.8 Hz, 1H), 8.24 (d, J = 9.8 Hz, 1H), 7.78-7.67 (m, 1H), 7.47 (d, J = 8.9 Hz, 1H), 7.36 (m, J = 20.7, 9.0 Hz, 3H), 3.83 (s, 3H), 3.70 (s, 1H), 3.25 (d, J = 12.3 Hz, 2H), 2.87 (s, 3H), 2.68 (s, 3H), 2.62 (d, J = 11.5 Hz, 2H), 2.00 (s, 1H), 1.83 (d, J = 14.9 Hz, 1H), 1.64 (d, J = 14.2 Hz, 1H), 1.33- 1.16 (m, 1H). |
| Example 71 | 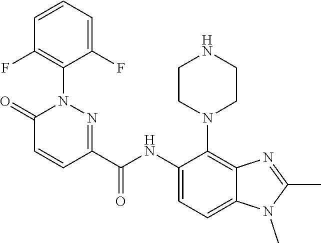 [SMILES: Cc1nc2c(N3CCNCC3)c(NC(=O)c3ccc(=O)n(-c4c(F)cccc4F)n3)ccc2n1C] 1-(2,6-difluorophenyl)-N-(1,2- dimethyl-4-(piperazin-1-yl)-1H- benzo[d]imidazol-5-yl)-6-oxo-1,6- dihydropyridazine-3-carboxamide | HCOOH salt | LC-MS m/z: 480.5(M + 1)⁺ ¹H NMR (400 MHz, CD₃OD) δ 8.45-8.37 (m, 1H), 8.23 (dd, J = 9.8, 1.2 Hz, 1H), 7.72 (tt, J = 8.6, 6.2 Hz, 1H), 7.46-7.35 (m, 2H), 7.34 (dd, J = 4.9, 2.9 Hz, 1H), 7.33-7.25 (m, 1H), 3.78 (s, 3H), 3.39 (s, 4H), 2.95 (s, 4H), 2.62 (s, 3H). |
| Example 72 | 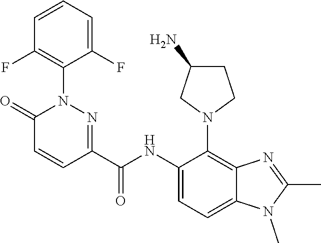 [SMILES: Cc1nc2c(N3CC[C@H](N)C3)c(NC(=O)c3ccc(=O)n(-c4c(F)cccc4F)n3)ccc2n1C] (S)-N-(4-(3-aminopyrrolidin-1-yl)- 1,2-dimethyl-1H-benzo[d]imidazol- 5-yl)-1-(2,6-difluorophenyl)-6-oxo- 1,6-dihydropyridazine-3- carboxamide | HCl salt | LC-MS m/z: 480.2 (M + 1)+ ¹H NMR (400 MHz, d₆-DMSO) δ 10.39 (s, 1H), 8.37 (s, 2H), 8.11 (d, J = 9.6 Hz, 1H), 7.90 (d, J = 8.8 Hz, 1H), 7.78-7.65 (m, 2H), 7.47-7.45 (m, 2H), 7.36 (d, J = 9.6 Hz, 1H), 3.88 (s, 3H), 3.74 (s, 3H), 3.32-3.30 (m, 1H), 3.27-3.09 (m, 1H), 2.83 (s, 3H), 2.02 (d, J = 6.4 Hz, 2H). |
| Example 73 | 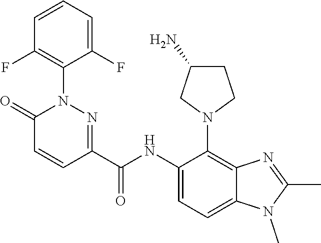 [SMILES: Cc1nc2c(N3CC[C@@H](N)C3)c(NC(=O)c3ccc(=O)n(-c4c(F)cccc4F)n3)ccc2n1C] (R)-N-(4-(3-aminopyrrolidin-1-yl)- 1,2-dimethyl-1H-benzo[d]imidazol- 5-yl)-1-(2,6-difluorophenyl)-6-oxo- 1,6-dihydropyridazine-3- carboxamide | HCl salt | LC-MS m/z: 480.3 (M + 1)+ ¹H NMR (400 MHz, CD₃OD) δ 8.22 (d, J = 9.9 Hz, 1H), 8.17 (d, J = 8.9 Hz, 1H), 7.81-7.60 (m, 2H), 7.39-7.27 (m, 3H), 3.97 (s, 3H), 3.89-3.81 (m, 2H), 3.78-3.72 (m, 2H), 3.51- 3.40 (m, 1H), 2.91 (s, 3H), 2.28-2.18 (m, 1H), 2.20-2.02 (m, 1H). |
| Example 74 | 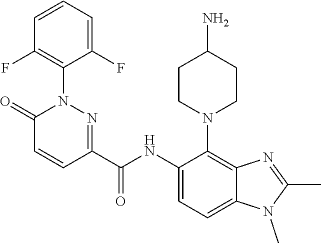 [SMILES: Cc1nc2c(N3CCC(N)CC3)c(NC(=O)c3ccc(=O)n(-c4c(F)cccc4F)n3)ccc2n1C] N-(4-(4-aminopiperidin-1-yl)-1,2- dimethyl-1H-benzo[d] 98 midazole- 5-yl)-1-(2,6-difluorophenyl)-6-oxo- 1,6-dihydropyridazine-3- carboxamide | HCl salt | LC-MS m/z: 494.2 (M + 1)+ ¹H NMR (400 MHz, CD₃OD) δ 8.39 (d, J = 8.7 Hz, 1H), 8.25- 8.23 (m, 1H), 7.76-7.64 (m, 2H), 7.40-7.30 (m, 3H), 3.95 (s, 3H), 3.46-3.40 (m, 2H), 3.27-3.25 (m, 1H), 3.16-3.10 (m, 2H), 2.88 (s, 3H), 1.88- 1,86 (m, 2H), 1.78-1,76 (m, 2H). |
| Example 75 | 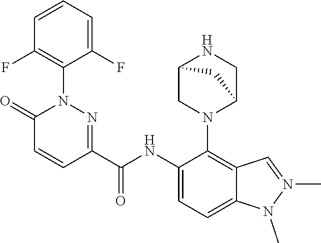 N-(4-((1S,4S)-2,5- diazabicyclo[2.2.1]heptan-2-yl)-1,2- dimethyl-1H-benzo[d]imidazol-5- yl)-1-(2,6-difluorophenyl)-6-oxo- 1,6-dihydropyridazine-3- carboxamide | HCl salt | LC-MS m/z: 492 (M + 1)⁺ ¹H NMR (400 MHz, CD₃OD) δ 8.13 (d, J = 9.9 Hz, 1H), 7.93 (d, J = 8.8 Hz, 1H), 7.63-7.53 (m, 1H), 7.46 (d, J = 8.9 Hz, 1H), 7.21 (t, J = 9.0 Hz, 3H), 4.43 (s, 1H), 4.26 (s, 1H), 4.01 (d, J = 10.4 Hz, 1H), 3.85 (s, 3H), 3.51-3.40 (m, 2H), 3.15 (d, J = 11.2 Hz, 1H), 2.81 (s, 3H), 2.06 (d, J = 11.2 Hz, 1H), 1.79 (d, J = 10.4 Hz, 1H). |
| Example 76 | 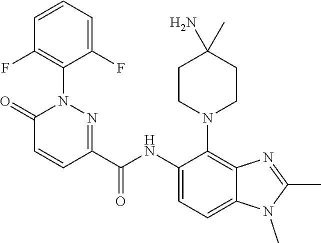 [SMILES: Cc1nc2c(N3CCC(C)(N)CC3)c(NC(=O)c3ccc(=O)n(-c4c(F)cccc4F)n3)ccc2n1C] N-(4-(4-amino-4-methylpiperidin-1- yl)-1,2-dimethyl-1H- benzo[d]imidazol-5-yl)-1-(2,6- difluorophenyl)-6-oxo-1,6- dihydropyridazine-3-carboxamide | HCl salt | LC-MS m/z: 508.2 (M + 1)+ ¹H NMR (400 MHz, d₆-DMSO) δ 10.38 (br, 1H), 8.40 (br, 5H), 8.18 (d, J = 9.9 Hz, 1H), 7.87- 7.67 (m, 1H), 7.48 (t, J = 8.4 Hz, 2H), 7.39 (d, J = 9.9 Hz, 1H), 3.84 (s, 3H), 3.70 (br, 2H), 2.84 (br, 5H), 1.80 (m, 2H), 1.38 (m, 2H), 0.95 (s, 3H). |
| Example 77 | 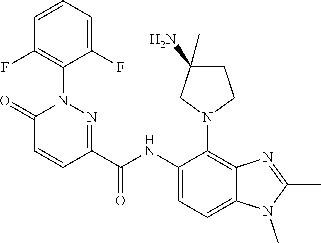 [SMILES: Cc1nc2c(N3CC[C@](C)(N)C3)c(NC(=O)c3ccc(=O)n(-c4c(F)cccc4F)n3)ccc2n1C] (S)-N-(4-(3-amino-3- methylpyrrolidin-1-yl)-1,2- dimethyl-1H-benzo[d]imidazol-5- yl)-1-(2,6-difluorophenyl)-6-oxo- 1,6-dihydropyridazine-3- carboxamide | HCl salt | LC-MS m/z: 494.2 (M + 1)+ ¹H NMR (400 MHz, CD₃OD) δ 8.19 (d, J = 9.0 Hz, 1H), 8.12 (d, J = 9.9 Hz, 1H), 7.64-7.51 (m, 2H), 7.26-7.14 (m, 3H), 3.85 (s, 3H), 3.86-3.79 (m, 1H), 3.55 (d, J = 10.0 Hz, 1H), 3.24 (s, 2H), 2.79 (s, 3H), 2.32- 2.17 (m, 1H), 2.00-1.84 (m, 1H), 1.18 (s, 3H). |
| Example 78 | 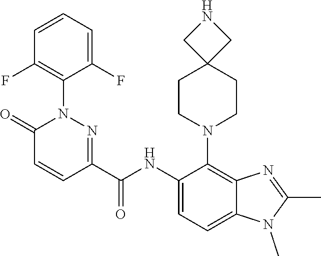 [SMILES: Cc1nc2c(N3CCC4(CC3)CNC4)c(NC(=O)c3ccc(=O)n(-c4c(F)cccc4F)n3)ccc2n1C] 1-(2,6-difluorophenyl)-N-(1,2- dimethyl-4-(2,7- diazaspiro[3.5]nonan-7-yl)-1H- benzo[d]imidazol-5-yl)-6-oxo-1,6- dihydropyridazine-3-carboxamide | HCl salt | LC-MS m/z: 520.3 (M + 1)+ ¹H NMR (400 MHz, CD₃OD) δ 8.82-8.80 (m, 1H), 8.27-8.25 (m, 1H), 7.73-7.71 (m, 2H), 7.39-7.37 (m, 2H), 7.33 (d, J = 9.9 Hz, 1H), 4.38 (s, 3H), 3.98 (s, 3H), 3.80 (s, 1H), 3.20- 3.03 (m, 4H), 2.89 (s, 3H), 1.82-1.79 (s, 4H). |
| Example 79 | 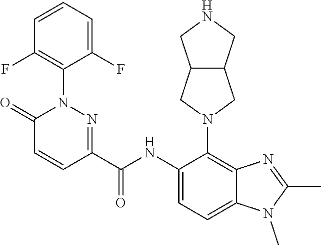 [SMILES: Cc1nc2c(N3CC4CNCC4C3)c(NC(=O)c3ccc(=O)n(-c4c(F)cccc4F)n3)ccc2n1C] 1-(2,6-difluorophenyl)-N-(4- (hexahydropyrrolo[3,4-c]pyrrol- 2(1H)-yl)-1,2-dimethyl-1H- benzo[d]imidazol-5-yl)-6-oxo-1,6- dihydropyridazine-3-carboxamide | HCl salt | LC-MS m/z: 506.5 (M + 1)⁺ ¹H NMR (400 MHz, CD₃OD) δ 8.55 (d, J = 9.0 Hz, 1H), 8.24 (d, J = 9.9 Hz, 1H), 7.75-7.70 (m, 2H), 7.34 (d, J = 9.9 Hz, 1H), 3.99 (s, 3H), 3.51-3.42 (m, 6H), 3.31-3.25 (m, 2H), 2.96 (s, 3H), 2.94-2.87 (m, 2H) |
| Example 80 | 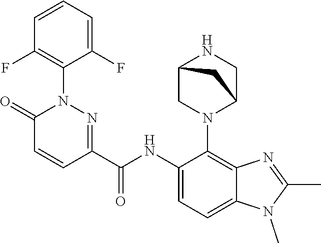 [SMILES: Cc1nc2c(N3C[C@H]4C[C@@H]3CN4)c(NC(=O)c3ccc(=O)n(-c4c(F)cccc4F)n3)ccc2n1C] N-(4-((1R,4R)-2,5- diazabicyclo[2.2.1]heptan-2-yl)-1,2- dimethyl-1H-benzo[d]imidazol-5- yl)-1-(2,6-difluorophenyl)-6-oxo- 1,6-dihydropyridazine-3- carboxamide | HCl salt | LC-MS m/z: 492.6 (M + 1)+ ¹H NMR: (400 MHz, CD₃OD) δ 8.25 (d, J = 9.9 Hz, 1H), 8.05 (d, J = 8.9 Hz, 1H), 7.73-7.66 (m, 1H), 7.59 (d, J = 8.9 Hz, 1H), 7.36-7.28 (m, 3H), 4.56 (s, 1H), 4.38 (s, 1H), 4.14 (d, J = 10.4 Hz, 1H), 3.97 (s, 3H), 3.63-3.46 (m, 2H), 3.29- 3.25 (m, 1H), 2.94 (s, 3H), 2.18 (d, J = 11.2 Hz, 1H), 1.91 (d, J = 11.2 Hz, 1H). |
| Example 81 | 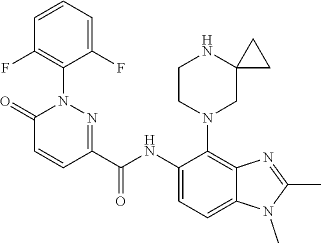 [SMILES: Cc1nc2c(N3CCNC4(CC4)C3)c(NC(=O)c3ccc(=O)n(-c4c(F)cccc4F)n3)ccc2n1C] 1-(2,6-difluorophenyl)-N-(1,2- dimethyl-4-(4,7- diazaspiro[2.5]octan-7-yl)-1H- benzo[d]imidazol-5-yl)-6-oxo-1,6- dihydropyridazine-3-carboxamide | HCl salt | LC-MS m/z: 506.6 (M + 1)⁺ ¹H NMR (400 MHz, CD₃OD) δ 8.57 (d, J = 8.9 Hz, 1H), 8.29 (d, J = 9.9 Hz, 1H), 7.73-7.64 (m, 2H), 7.39-7.33 (m, 3H), 3.93 (s, 3H), 3.62 (br, 2H), 3.38-3.33 (m, 2H), 3.32- 3.28 (m, 2H), 2.84 (s, 3H), 0.85 (s, 2H), 0.71 (br, 2H). |
| Example 82 | 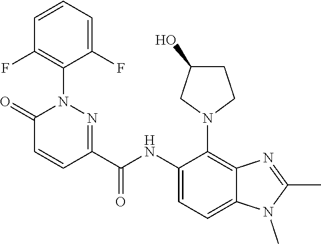 [SMILES: Cc1nc2c(N3CC[C@H](O)C3)c(NC(=O)c3ccc(=O)n(-c4c(F)cccc4F)n3)ccc2n1C] (S)-1-(2,6-difluorophenyl)-N-(4-(3- hydroxypyrrolidin-1-yl)-1,2- dimethyl-1H-benzo[d]imidazol-5- yl)-6-oxo-1,6-dihydropyridazine-3- carboxamide | HCl salt | LC-MS m/z: 481.3 (M + 1)⁺ ¹H NMR (400 MHz, d₆-DMSO) δ 10.37 (s, 1H), 8.12 (d, J = 9.8 Hz, 1H), 8.08 (d, J = 8.7 Hz, 1H), 7.72 (m, 1H), 7.45 (t, J = 8.5 Hz, 2H), 7.35 (d, J = 9.8 Hz, 1H), 7.26 (d, J = 8.7 Hz, 1H), 5.41 (s, 1H), 4.16 (s, 1H), 3.70 (s, 3H), 3.49 (m, 2H), 3.30- 3.26 (m, 1H), 3.19-3.11 (m, 1H), 2.52 (s, 3H), 1.78-1.65 (m, 2H). |
| Example 83 | 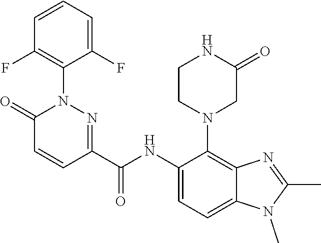 1-(2,6-difluorophenyl)-N-(1,2- dimethyl-4-(3-oxopiperazin-1-yl)- 1H-benzo[d]imidazol-5-yl)-6-oxo- 1,6-dihydropyridazine-3- carboxamide | CHCOOH salt | LC-MS m/z: 449.2 (M + 1)⁺ ¹H NMR (400 MHz, d₆-DMSO) δ 10.42 (s, 1H), 8.26 (d, J = 8.7 Hz, 1H), 8.12 (d, J = 9.8 Hz, 1H), 7.74-7.67 (m, 2H), 7.46- 7.32 (m, 4H), 3.81 (s, 2H), 3.71 (s, 3H), 3.28-3.26 (m, 2H), 2.99 (s, 2H), 2.52 (s, 3H). |
| Example 84 | 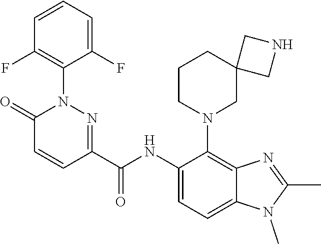 [SMILES: Cc1nc2c(N3CCCC4(CNC4)C3)c(NC(=O)c3ccc(=O)n(-c4c(F)cccc4F)n3)ccc2n1C] 1-(2,6-difluorophenyl)-N-(1,2- dimethyl-4-(2,6- diazaspiro[3.5]nonan-6-yl)-1H- benzo[d]imidazol-5-yl)-6-oxo-1,6- dihydropyridazine-3-carboxamide | HCl salt | LC-MS m/z: 520.3 (M + 1)⁺ ¹H NMR (400 MHz, d₆-DMSO) δ 10.29-10.18 (m, 1H), 8.54 (br, 1H), 8.21-8.19 (m, 1H), 7.86-7.69 (m, 2H), 7.58- 7.46 (m, 2H), 7.42 (d, J = 9.9 Hz, 1H), 3.86 (s, 3H), 3.62- 3.48 (m, 2H), 2.92-2.90 (m, 2H), 2.74-2.71 (m, 2H), 2.70- 2.68 (m, 2H), 2.53-2.51 (m, 2H), 2.14 (s, 3H), 1.71-1.55 (m, 2H). |
| Example 85 | 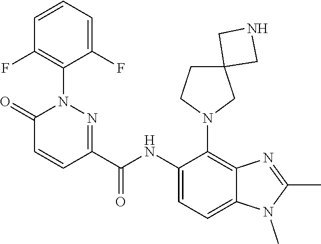 [SMILES: Cc1nc2c(N3CCC4(CNC4)C3)c(NC(=O)c3ccc(=O)n(-c4c(F)cccc4F)n3)ccc2n1C] 1-(2,6-difluorophenyl)-N-(1,2- dimethyl-4-(2,6- diazaspiro[3.4]octan-6-yl)-1H- benzo[d]imidazol-5-yl)-6-oxo-1,6- dihydropyridazine-3-carboxamide | HCl salt | LC-MS m/z: 506.3 (M + 1)+ ¹H NMR (400 MHz, CD₃OD) δ 8.36 (d, J = 8.8 Hz, 1H), 8.24 (d, J = 9.9 Hz, 1H), 7.74-7.32 (m, 1H), 7.43-7.25 (m, 4H), 3.97 (d, J = 11.2 Hz, 2H), 3.78 (s, 3H), 3.77 (d, J = 11.2 Hz, 2H), 3.69 (s, 2H), 3.36-3.33 (m, 2H), 2.61 (s, 3H), 2.19 (t, J = 7.1 Hz, 2H). |
| Example 86 | 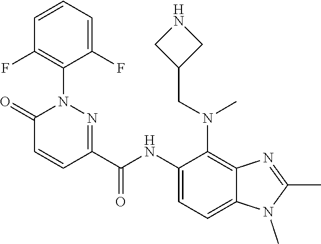 [SMILES: Cc1nc2c(N(C)CC3CNC3)c(NC(=O)c3ccc(=O)n(-c4c(F)cccc4F)n3)ccc2n1C] N-(4-((azetidin-3- ylmethyl)(methyl)amino)-1,2- dimethyl-1H-benzo[d]imidazol-5- yl)-1-(2,6-difluorophenyl)-6-oxo- 1,6-dihydropyridazine-3- carboxamide | HCl salt | LC-MS m/z: 494.2 (M + 1)⁺ ¹H NMR (400 MHz, CD₃OD) δ 8.43 (d, J = 8.8 Hz, 1H), 8.24 (d, J = 9.6 Hz, 1H), 7.81-7.66 (m, 1H), 7.37-7.30 (m, 4H), 3.77 (s, 3H), 3.55 (d, J = 7.6 Hz, 2H), 3.52-3.47 (m, 2H), 3.28-3.25 (m, 2H), 2.81 (s, 3H), 2.66-2.64 (m, 1H), 2.61 (s, 3H). |
| Example 87 | 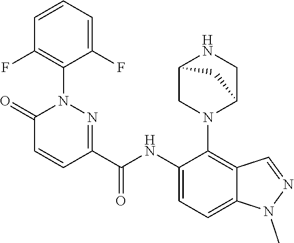 [SMILES: Cn1ncc2c(N3C[C@@H]4C[C@H]3CN4)c(NC(=O)c3ccc(=O)n(-c4c(F)cccc4F)n3)ccc21] N-(4-((1S,4S)-2,5- diazabicyclo[2.2.1]heptan-2-yl)-1- methyl-1H-indazol-5-yl)-1-(2,6- difluorophenyl)-6-oxo-1,6- dihydropyridazine-3-carboxamide | HCl Salt | LC-MS m/z: 478.5 (M + 1)⁺ ¹H NMR (400 MHz, CD₃OD) δ 8.09 (d, J = 9.8 Hz, 1H), 8.02 (s, 1H), 7.59-7.50 (m, 1H), 7.30 (d, J = 8.8 Hz, 1H), 7.19- 7.16 (m, 3H), 6.99 (d, J = 8.8 Hz, 1H), 4.82 (s, 1H), 4.34 (s, 1H), 3.95-3.88 (m, 4H), 3.45 (d, J = 11.4 Hz, 1H), 3.37-3.35 (m, 2H), 2.18 (d, J = 11.0 Hz, 1H), 1.94 (d, J = 11.0 Hz, 1H). |
| Example 88 | 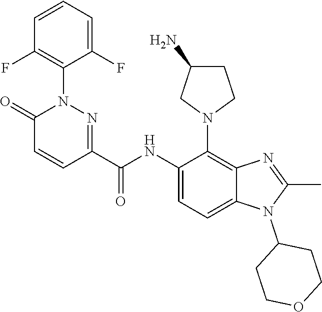 [SMILES: Cc1nc2c(N3CC[C@H](N)C3)c(NC(=O)c3ccc(=O)n(-c4c(F)cccc4F)n3)ccc2n1C1CCOCC1] (S)-N-(4-(3-aminopyrrolidin-1-yl)-2- methyl-1-(tetrahydro-2H-pyran-4- yl)-1H-benzo[d]imidazol-5-yl)-1- (2,6-difluorophenyl)-6-oxo-1,6- dihydropyridazine-3-carboxamide | HCOOH Salt | LC-MS m/z: 550.2 (M + 1)⁺ ¹H NMR (400 MHz, CD₃OD) δ 8.51 (s, 1H), 8.25-8.20 (m, 2H), 7.71-7.63 (m, 1H), 7.60 (d, J = 5.3 Hz, 1H), 7.31 (m, 3H), 4.63-4.58 (s, 2H), 4.16- 4.12 (m, 2H), 3.75-3.39 (m, 6H), 2.68 (s, 3H), 2.60-2.52 (m, 2H), 2.21-2.09 (m, 2H), 1.87-1.84 (m, 2H). |
| Example 89 | 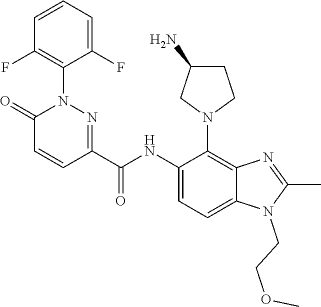 [SMILES: COCCn1c(C)nc2c(N3CC[C@H](N)C3)c(NC(=O)c3ccc(=O)n(-c4c(F)cccc4F)n3)ccc21] (S)-N-(4-(3-aminopyrrolidin-1-yl)-1- (2-methoxyethyl)-2-methyl-1H- benzo[d]imidazol-5-yl)-1-(2,6- difluorophenyl)-6-oxo-1,6- dihydropyridazine-3-carboxamide | HCl Salt | LC-MS m/z: 524.5 (M + 1)⁺ ¹H NMR (400 MHz, CD₃OD) δ 8.26-8.18 (d, J = 12 Hz, 1H), 8.15 (d, J = 9.0 Hz, 1H), 7.77- 7.61 (m, 2H), 7.41-7.26 (m, 3H), 4.65 (t, J = 4.8 Hz, 2H), 3.90-3.85 (m, 1H), 3.83- 3.68 (m, 4H), 3.46 (dd, J = 10.3, 3.9 Hz, 1H), 3.38-3.33 (m, 1H), 3.31 (s, 3H), 2.96 (s, 3H), 3.33-3.23 (m, 1H), 2.20- 2.10 (m, 1H). |
| Example 90 | 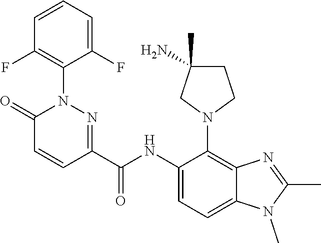 [SMILES: Cc1nc2c(N3CC[C@@](C)(N)C3)c(NC(=O)c3ccc(=O)n(-c4c(F)cccc4F)n3)ccc2n1C] (R)-N-(4-(3-amino-3- methylpyrrolidin-1-yl)-1,2- dimethyl-1H-benzo[d]imidazol-5- yl)-1-(2,6-difluorophenyl)-6-oxo- 1,6-dihydropyridazine-3- carboxamide | HCl salt | LC-MS m/z: 494.2 (M + 1)⁺ ¹H NMR (400 MHz, CD₃OD) δ 8.35 (d, J = 9.0 Hz, 1H), 8.25 (d, J = 9.9 Hz, 1H), 7.70 (tt, J = 8.7, 3.4 Hz, 2H), 7.41-7.22 (m, 3H), 3.98 (s, 3H), 3.85 (q, J = 8.4 Hz, 1H), 3.70 (d, J = 9.9 Hz, 1H), 3.40-3.35 (m, 1H), 2.93 (s, 3H), 2.39 (ddd, J = 12.3, 8.1, 3.5 Hz, 1H), 1.96 (dt, J = 13.9, 8.4 Hz, 1H), 1.29 (s, 3H). |
| Example 91 | 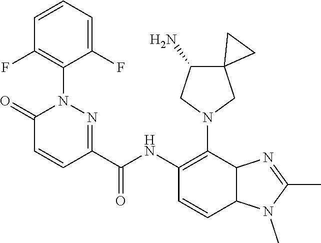 [SMILES: CC1=NC2C(N3C[C@H](N)C4(CC4)C3)=C(NC(=O)c3ccc(=O)n(-c4c(F)cccc4F)n3)C=CC2N1C] (R)-N-(4-(7-amino-5- azaspiro[2.4]heptan-5-yl)-1,2- dimethyl-1H-benzo[d]imidazol-5- yl)-1-(2,6-difluorophenyl)-6-oxo- 1,6-dihydropyridazine-3- carboxamide | HCl salt | LC-MS m/z: 506.5 (M + 1)⁺ ¹H NMR (400 MHz, CD₃OD) δ 8.24 (d, J = 9.9 Hz, 1H), 8.14 (d, J = 8.9 Hz, 1H), 7.76-7.63 (m, 2H), 7.39-7.28 (m, 3H), 4.06 (dd, J = 10.1, 5.6 Hz, 2H), 3.98 (s, 3H), 3.53 (dd, J = 10.7, 2.5 Hz, 1H), 3.36 (d, J = 2.4 Hz, 1H), 2.95 (d, J = 8.6 Hz, 1H), 2.93 (s, 3H), 1.08 (dt, J = 10.1, 5.9 Hz, 1H), 0.86 (dt, J = 9.3, 6.1 Hz, 1H), 0.65-0.50 (m, 2H) |
| Example 92 | 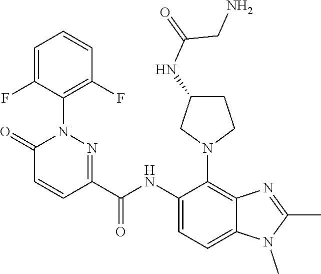 [SMILES: Cc1nc2c(N3CC[C@@H](NC(=O)CN)C3)c(NC(=O)c3ccc(=O)n(-c4c(F)cccc4F)n3)ccc2n1C] (R)-N-(4- (3-(2-aminoacetamido)pyrrolidin-1- yl)-1,2-dimethyl-1H- benzo[d]imidazol-5-yl)-1-(2,6- difluorophenyl)-6-oxo-1,6- dihydropyridazine-3-carboxamide | HCOOH salt | LC-MS m/z: 537.6 (M + 1)⁺ ¹H NMR (400 MHz, CD₃OD) δ 8.33 (d, J = 8.8 Hz, 1H), 8.22 (d, J = 9.9 Hz, 1H), 7.69 (tt, J = 8.7, 6.2 Hz, 1H), 7.41-7.27 (m, 4H), 4.39 (td, J = 6.0, 3.1 Hz, 1H), 3.79 (s, 3H), 3.73 (d, J = 5.0 Hz, 2H), 3.56 (dd, J = 10.4, 6.3 Hz, 1H), 3.43 (td, J = 10.3, 9.9, 2.6 Hz, 2H), 3.19 (ddd, J = 8.8, 6.8, 3.6 Hz, 1H), 2.64 (s, 3H), 1.91 (dq, J = 8.8, 6.0, 5.1 Hz, 2H). |
| Example 93 | 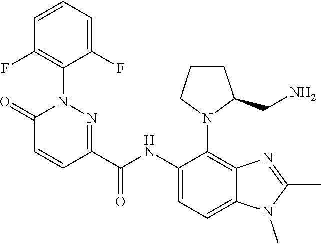 [SMILES: Cc1nc2c(N3CCC[C@H]3CN)c(NC(=O)c3ccc(=O)n(-c4c(F)cccc4F)n3)ccc2n1C] (S)-N-(4-(2- (aminomethyl)pyrrolidin-1-yl)-1,2- dimethyl-1H-benzo[d]imidazol-5- yl)-1-(2,6-difluorophenyl)-6-oxo- 1,6-dihydropyridazine-3- carboxamide | HCOOH salt | LC-MS m/z: 494.2 (M + 1)⁺ ¹H NMR (400 MHz, CD₃OD) δ 8.21 (d, J = 9.9 Hz, 1H), 7.97 (d, J = 8.7 Hz, 1H), 7.68 (tt, J = 8.5, 6.2 Hz, 1H), 7.37-7.25 (m, 4H), 4.71-4.57 (m, 2H), 3.77 (s, 3H), 3.26-3.17 (m, 1H), 2.74 (qd, J = 12.6, 5.7 Hz, 2H), 2.59 (s, 3H), 2.32 (dq, J = 14.6, 7.5 Hz, 1H), 1.95 (h, J = 6.7 Hz, 1H), 1.72 (dq, J = 14.0, 7.5 Hz, 1H), 1.66-1.55 (m, 1H). |
| Example 94 | 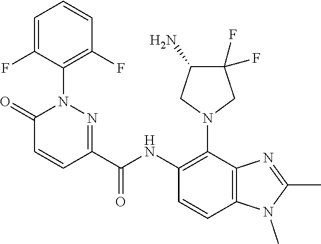 [SMILES: Cc1nc2c(N3C[C@H](N)C(F)(F)C3)c(NC(=O)c3ccc(=O)n(-c4c(F)cccc4F)n3)ccc2n1C] (S)-N-(4-(4-amino-3,3- difluoropyrrolidin-1-yl)-1,2- dimethyl-1H-benzo[d]imidazol-5- yl)-1-(2,6-difluorophenyl)-6-oxo- 1,6-dihydropyridazine-3- carboxamide | HCOOH salt | LC-MS m/z: 516.2 (M + 1)⁺ ¹H NMR (400 MHz, CD₃OD) δ 8.32-8.24 (m, 1H), 8.11 (d, J = 9.8 Hz, 1H), 7.56 (tt, J = 6.3, 8.6 Hz, 1H), 7.26-7.12 (m, 4H), 3.88 (dt, J = 11.0, 15.0 Hz, 1H), 3.66 (s, 3H), 3.59-3.44 (m, 2H), 3.43-3.25 (m, 2H), 2.50 (s, 3H). |
| Example 95 | 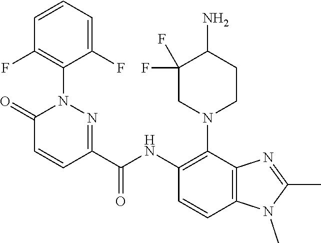 [SMILES: Cc1nc2c(N3CCC(N)C(F)(F)C3)c(NC(=O)c3ccc(=O)n(-c4c(F)cccc4F)n3)ccc2n1C] N-(4-(4- amino-3,3-difluoropiperidin-1-yl)- 1,2-dimethyl-1H-benzo[d]imidazol- 5-yl)-1-(2,6-difluorophenyl)-6-oxo- 1,6-dihydropyridazine-3- carboxamide | HCl salt | LC-MS m/z: 530.2 (M + 1)⁺ ¹H NMR (400 MHz, CD₃OD) δ 8.39 (d, J = 8.8 Hz, 1H), 8.25 (d, J = 9.8 Hz, 1H), 7.69 (td, J = 8.5, 4.2 Hz, 1H), 7.42 (d, J = 8.8 Hz, 1H), 7.32 (dd, J = 9.5, 6.4 Hz, 3H), 3.81 (s, 3H), 3.62 (s, 2H), 3.46 (s, 1H), 3.23 (q, J = 7.4 Hz, 1H), 2.65 (s, 3H), 1.95 (s, 1H), 1.78 (s, 1H), 1.33 (dd, J = 12.4, 5.1 Hz, 1H). |
| Example 96 | 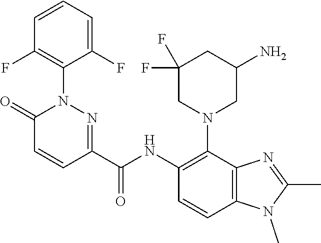 [SMILES: Cc1nc2c(N3CC(N)CC(F)(F)C3)c(NC(=O)c3ccc(=O)n(-c4c(F)cccc4F)n3)ccc2n1C] N-(4-(5- amino-3,3-difluoropiperidin-1-yl)- 1,2-dimethyl-1H-benzo[d]imidazol- 5-yl)-1-(2,6-difluorophenyl)-6-oxo- 1,6-dihydropyridazine-3- carboxamide | HCl salt | LC-MS m/z: 530.2 (M + 1)⁺ ¹H NMR (400 MHz, CD₃OD) δ 8.24 (d, J = 9.9 Hz, 1H), 8.15 (s, 1H), 7.68 (ddd, J = 14.7, 8.6, 6.2 Hz, 1H), 7.42 (d, J = 8.9 Hz, 1H), 7.31 (dd, J = 9.5, 2.3 Hz, 3H), 3.80 (s, 3H), 3.75- 3.66 (m, 1H), 3.64-3.54 (m, 1H), 3.38 (s, 1H), 3.22 (t, J = 7.9 Hz, 1H), 2.64 (s, 3H), 2.32 (d, J = 11.5 Hz, 1H), 2.16 (m, 1H), 1.49 (dd, J = 6.6, 3.1 Hz, 1H). |

### Example 97: N-{4-[(35)-3-aminopyrrolidin-1-yl]-2-cyclopropyl-1-methyl-1,3-benzodiazol-5-yl}-1-(2,6-difluorophenyl)-6-oxopyridazine-3-carboxamide

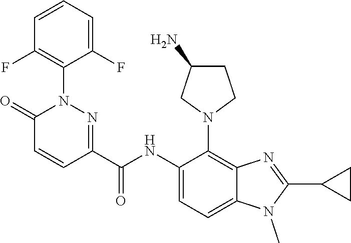 [SMILES: Cn1c(C2CC2)nc2c(N3CC[C@H](N)C3)c(NC(=O)c3ccc(=O)n(-c4c(F)cccc4F)n3)ccc21]

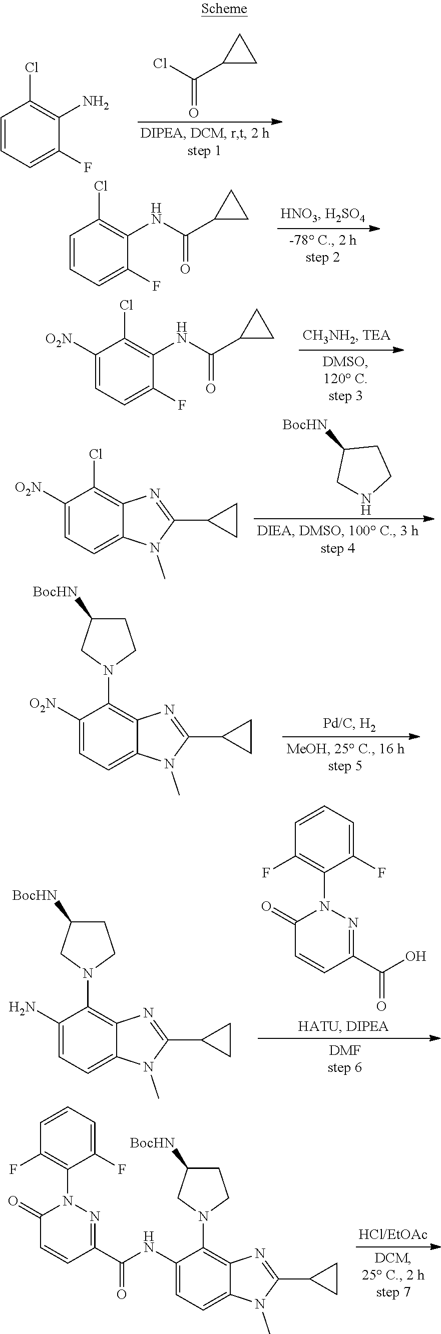

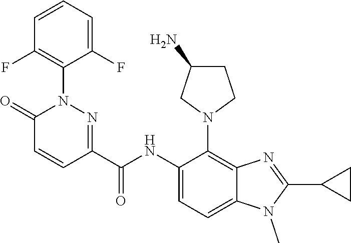 [SMILES: Cn1c(C2CC2)nc2c(N3CC[C@H](N)C3)c(NC(=O)c3ccc(=O)n(-c4c(F)cccc4F)n3)ccc21]

### Step 1: N-(2-chloro-6-fluorophenyl)cyclopropanecarboxamide

To a solution of 2-chloro-6-fluoroaniline (2.0 g, 13.7 mmol, 1.0 eq) and DIPEA (3.4 mL, 20.55 mmol, 1.5 eq) in DCM (20 mL) was added a solution of cyclopropanecarbonyl chloride (1.48 mL, 16.44 mmol, 1.2 eq) in DCM (10 mL) dropwise at 0° C. The reaction mixture was stirred at the same temperature for 2 hours. The mixture was concentrated under reduced pressure and the residue was purified by flash column chromatography (eluting with petroleum ether/EtOAc=1/1) to give N-(2-chloro-6-fluorophenyl)cyclopropanecarboxamide (2.2 g, Yield: 71.5%) as a white solid.

LC-MS m/z: 214.1 (M+1)⁺.

### Step 2: N-(2-chloro-6-fluoro-3-nitrophenyl)cyclopropanecarboxamide

To a solution of N-(2-chloro-6-fluorophenyl)cyclopropanecarboxamide (2.2 g, 10.3 mmol, 1.0 eq) in H₂SO₄ (10 mL) was added a solution of concentrated nitric acid (0.6 mL, 11.6 mmol, 1.13 eq) dropwise at −55° C. The reaction mixture was continued to stir at the same temperature for 2 hours. The resulting mixture was warmed to room temperature, then quenched with ice-water (30 mL) to obtain N-(2-chloro-6-fluoro-3-nitrophenyl) cyclopropanecarboxamide (2 g, Yield: 70.9%) as a yellow solid.

LC-MS m/z: 259.1 (M+1)⁺.

### Step 3: 4-chloro-2-cyclopropyl-1-methyl-5-nitro-1,3-benzodiazole

To a mixture of N-(2-chloro-6-fluoro-3-nitrophenyl)cyclopropanecarboxamide (500 mg, 1.93 mmol, 1 eq.) and TEA (1 mL) in DMSO (5 mL) was added a solution of CH₃NH₂ in THF (2M, 72 mg, 3.83 mmol, 1.5 eq.) at room temperature. The reaction was continued to stir at room temperature for 4 hours. The reaction was quenched with water (20 mL) and extracted with ethyl acetate (50 mL×2). The combined organic phase was washed with brine (50 mL×3), dried over Na₂SO₄, filtrated, concentrated under reduced pressure to give a residue, which was purified by flash column chromatography (eluting with Petroleum ether/EtOAc=2/1) to afford 4-chloro-2-cyclopropyl-1-methyl-5-nitro-1,3-benzodiazole (210 mg, Yield: 42.8%) as a yellow solid.

LC-MS m/z: 252.0 (M+1)⁺.

### Step 4: tert-butyl N-[(35)-1-(2-cyclopropyl-1-methyl-5-nitro-1,3-benzodiazol-4-yl)pyrrolidin-3-yl]carbamate

To a solution of 4-chloro-2-(methoxymethyl)-1-methyl-5-nitro-1H-benzo[d]imidazole (100 mg, 0.40 mmol, 1.0 eq) and tert-butyl (S)-pyrrolidin-3-ylcarbamate (89 mg, 0.48 mmol, 1.2 eq) in DMSO (1.5 mL) was added diisopropylethylamine (103 mg, 0.79 mmol, 2 eq). The reaction mixture was stirred at 100° C. for 4 hours. After cooling to room temperature, the reaction was quenched with water and extracted with EtOAc (10 mL×2). The combined organic phase was washed with brine (10 mL×3), dried over Na₂SO₄, filtrated and concentrated under reduced pressure to give a residue, which was purified by flash column chromatography (eluting with Petroleum ether/EtOAc=3/1) to afford tert-butyl N-[(35)-1-(2-cyclopropyl-1-methyl-5-nitro-1,3-benzodiazol-4-yl)pyrrolidin-3-yl]carbamate (130 mg, Yield: 77.4%) as a yellow solid.

LC-MS m/z: 402.1 (M+1)⁺.

### Step 5: tert-butyl (S)-(1-(5-amino-2-(methoxymethyl)-1-methyl-1H-benzo[d]imidazol-4-yl) pyrrolidin-3-yl) carbamate

The mixture of Pd/C (60 mg, 10%) and tert-butyl N-[(35)-1-(2-cyclopropyl-1-methyl-5-nitro-1,3-benzodiazol-4-yl)pyrrolidin-3-yl]carbamate (100 mg, 0.25 mmol) in methanol (5 mL) was hydrogenated under 15 psi for 2 hours. The reaction mixture was filtered and concentrated under reduced pressure to afford tert-butyl (S)-(1-(5-amino-2-(methoxymethyl)-1-methyl-1H-benzo[d]imidazol-4-yl) pyrrolidin-3-yl) carbamate (100 mg, Yield: 70.7%) as a white solid.

LC-MS m/z: 372.3 (M+1)⁺.

### Step 6: tert-butyl N-[(35)-1-{2-cyclopropyl-5-[1-(2,6-difluorophenyl)-6-oxopyridazine-3-amido]-1-methyl-1,3-benzodiazol-4-yl}pyrrolidin-3-yl]carbamate

To a mixture of tert-butyl (S)-(1-(5-amino-2-(methoxymethyl)-1-methyl-1H-benzo[d]imidazol-4-yl)pyrrolidin-3-yl)carbamate (80 mg, 0.21 mmol, 1.2 eq) and 1-(2,6-difluorophenyl)-6-oxo-1,6-dihydropyridazine-3-carboxylic acid (50 mg, 0.18 mmol, 1.0 eq) in DMF (2 mL) were added HATU (86 mg, 0.22 mmol, 1.5 eq) and DIPEA (0.14 mL, 0.63 mmol, 3.0 eq). The reaction mixture was stirred at 50° C. for 2 hours. After cooling to room temperature, the reaction was quenched with water (50 mL) and extracted with EtOAc (10 mL×3). The combined organic phase was washed with brine (50 mL), dried over Na₂SO₄, filtrated and concentrated under reduced pressure to give crude product, which was purified by SGC (UV254, Petroleum ether:EtOAc=3:1) to afford tert-butyl N-[(35)-1-{2-cyclopropyl-5-[1-(2,6-difluorophenyl)-6-oxopyridazine-3-amido]-1-methyl-1,3-benzodiazol-4-yl}pyrrolidin-3-yl]carbamate (55 mg, Yield: 40.1%) as a yellow solid.

LC-MS m/z: 606.3 (M+1)⁺.

### Step 7: N-{4-[(35)-3-aminopyrrolidin-1-yl]-2-cyclopropyl-1-methyl-1,3-benzodiazol-5-yl}-1-(2,6-difluorophenyl)-6-oxopyridazine-3-carboxamide

To a solution of tert-butyl N-[(35)-1-{2-cyclopropyl-5-[1-(2,6-difluorophenyl)-6-oxo pyridazine-3-amido]-1-methyl-1,3-benzodiazol-4-yl}pyrrolidin-3-yl]carbamate (35 mg, 0.052 mmol, 1.0 eq) in DCM (3 mL) was added HCl in EtOAC (0.5 mL). The reaction mixture was stirred at 25° C. for 2 hours. The reaction mixture was concentrated and washed with tert-butyl methyl ether to afford Example 97 (29.7 mg, Yield: 59.5%) as a yellow solid.

LC-MS m/z: 506.3 (M+1)⁺.

¹H NMR (400 MHz, CD₃OD) δ 8.22 (d, J=9.9 Hz, 1H), 8.11 (d, J=8.6 Hz, 1H), 7.73-7.64 (m, 2H), 7.36-7.32 (m, 3H), 4.08 (s, 3H), 3.84-3.70 (m, 3H), 3.46-3.37 (m, 1H), 3.29-3.26 (m, 1H), 2.50-2.40 (m, 1H), 2.28-2.13 (m, 2H), 1.58 (m, 2H), 1.47-1.45 (m, 2H).

### Example 98: N-{4-[(3S)-3-aminopyrrolidin-1-yl]-2-tert-butyl-1-methyl-1,3-benzodiazol-5-yl}-1-(2,6-difluorophenyl)-6-oxopyridazine-3-carboxamide

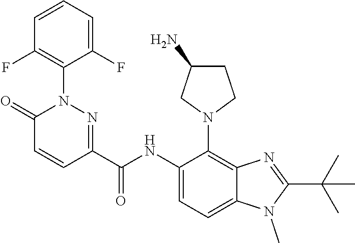 [SMILES: Cn1c(C(C)(C)C)nc2c(N3CC[C@H](N)C3)c(NC(=O)c3ccc(=O)n(-c4c(F)cccc4F)n3)ccc21]

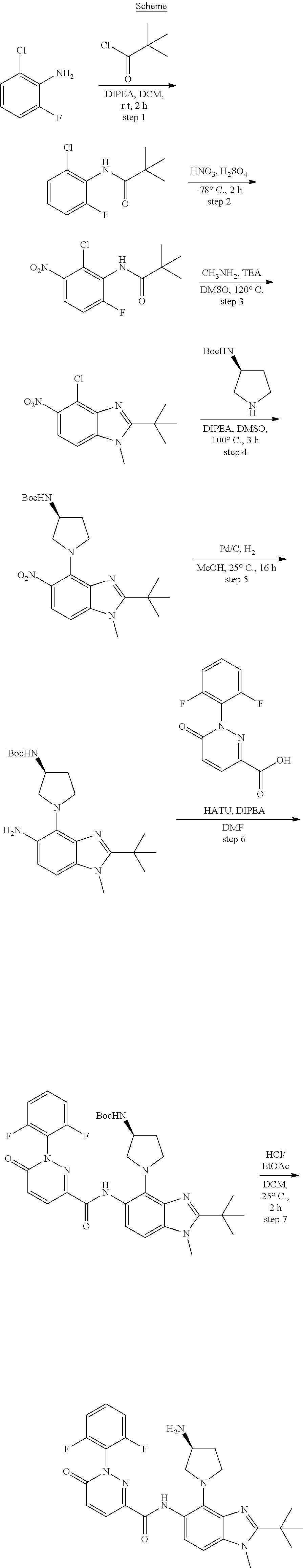

### Step 1: N-(2-chloro-6-fluorophenyl)-2,2-dimethylpropanamide

To a solution of 2-chloro-6-fluoroaniline (2.0 g, 13.7 mmol, 1.0 eq) and DIPEA (3.4 mL, 20.55 mmol, 1.5 eq) in DCM (20 mL) was added dropwise a solution of 2,2-dimethylpropanoyl chloride (1.98 g, 16.44 mmol, 1.2 eq) in DCM (10 mL) at 0° C. The reaction mixture was stirred at room temperature for 2 hours. The reaction mixture was concentrated under reduced pressure to obtain the crude product, which was purified by flash column chromatography (eluting with Petroleum ether/EtOAc=3/1) to give N-(2-chloro-6-fluorophenyl)pivalamide (2.4 g, Yield: 55.3%) as a white solid.

LC-MS m/z: 230.2 (M+1)⁺.

### Step 2: N-(2-chloro-6-fluoro-3-nitrophenyl)-2,2-dimethylpropanamide

To a solution of N-(2-chloro-6-fluorophenyl)-2,2-dimethylpropanamide (2.4 g, 10.4 mmol, 1.0 eq) in H₂SO₄ (10 mL) was added a solution of concentrated nitric acid (0.6 mL, 11.6 mmol, 1.12 eq) in H₂SO₄ (2 mL) dropwise at −55° C. The reaction mixture was continued to stir at the same temperature for 2 hours. The resulting mixture was warmed to room temperature, then quenched with ice-water (30 mL) to obtain N-(2-chloro-6-fluoro-3-nitrophenyl)pivalamide (2.8 g, Yield: 88.5%) as a yellow solid.

LC-MS m/z: 275.1 (M+1)⁺.

### Step 3: 4-chloro-2-cyclopropyl-1-methyl-5-nitro-1,3-benzodiazole

To a mixture of N-(2-chloro-6-fluoro-3-nitrophenyl)-2,2-dimethylpropanamide (500 mg, 1.82 mmol, 1 eq.) and TEA (388 mg, 3.83 mmol, 2 eq.) in DMSO (5 mL) was added a solution of CH₃NH₂ in THF (2M, 113 mg, 3.64 mmol, 2 eq.) at room temperature. The reaction was continued to stir for 2 hours. The mixture was diluted with water (20 mL) and extracted with ethyl acetate (3×50 mL). The combined organic phase was washed with brine (50 mL×3), dried over Na₂SO₄, filtrated, concentrated under reduced pressure to give a residue, which was purified by flash column chromatography (eluting with Petroleum ether/EtOAc=3/1) to afford 4-chloro-2-cyclopropyl-1-methyl-5-nitro-1,3-benzodiazole (150 mg, Yield: 43.6%) as a yellow solid.

LC-MS m/z: 268.0 (M+1)⁺.

### Step 4: tert-butyl N-[(35)-1-(2-tert-butyl-1-methyl-5-nitro-1,3-benzodiazol-4-yl)pyrrolidin-3-yl]carbamate

A mixture of 4-chloro-2-cyclopropyl-1-methyl-5-nitro-1,3-benzodiazole (150 mg, 0.56 mmol, 1.0 eq), diisopropylethylamine (290 mg, 2.24 mmol, 4 eq) and tert-butyl (S)-pyrrolidin-3-yl-carbamate (88 mg, 0.43 mmol, 1.2 eq) in DMSO was stirred at 100° C. for 4 hours. After cooling to room temperature, the reaction was quenched with water and extracted with EtOAc (10 mL×2). The combined organic phase was washed with brine (10 mL×3), dried over Na₂SO₄, filtrated and concentrated under reduced pressure to give a residue, which was purified by SGC (UV254, Petroleum ether:EtOAc=3:1) to afford tert-butyl N-[(35)-1-(2-tert-butyl-1-methyl-5-nitro-1,3-benzodiazol-4-yl)pyrrolidin-3-yl]carbamate (190 mg, Yield: 77.2%) as a yellow solid.

LC-MS m/z: 418.2 (M+1)⁺.

### Step 5: tert-butyl N-[(35)-1-(5-amino-2-tert-butyl-1-methyl-1,3-benzodiazol-4-yl)pyrrolidin-3-yl]carbamate

The mixture of Pd/C (79 mg, 0.25 mmol, 10%) and tert-butyl N-[(35)-1-(2-tert-butyl-1-methyl-5-nitro-1,3-benzodiazol-4-yl)pyrrolidin-3-yl]carbamate (190 mg, 0.46 mmol) in methanol (5 mL) was hydrogenated under 15 psi for 2 hours. The reaction mixture was filtered and concentrated to afford tert-butylN-[(35)-1-(5-amino-2-tert-butyl-1-methyl-1,3-benzodiazol-4-yl)pyrrolidin-3-yl]carbamate (120 mg, Yield: 54.4%) as a white solid.

LC-MS m/z: 388.2 (M+1)⁺.

### Step 6: tert-butyl N-[(35)-1-{2-tert-butyl-5-[1-(2,6-difluorophenyl)-6-oxopyridazine-3-amido]-1-methyl-1,3-benzodiazol-4-yl}pyrrolidin-3-yl]carbamate

A mixture of tert-butyl N-[(35)-1-(5-amino-2-tert-butyl-1-methyl-1,3-benzodiazol-4-yl)pyrrolidin-3-yl]carbamate (80 mg, 0.20 mmol, 1.0 eq) and 1-(2,6-difluorophenyl)-6-oxo-1,6-dihydropyridazine-3-carboxylic acid (50 mg, 0.20 mmol, 1 eq) in DMF (2 mL) were added HATU (86 mg, 0.22 mmol, 1.5 eq) and DIPEA (0.12 mL, 0.60 mmol, 3.0 eq). The mixture was stirred at 50° C. for 2 hours. After cooling to room temperature, the reaction was quenched with water (50 mL) and extracted with EtOAc (10 mL×3). The combined organic phase was washed with brine (50 mL), dried over Na₂SO₄, filtrated and concentrated under reduced pressure to give crude product, which was purified by SGC (UV254, Petroleum ether:EtOAc=3:1) to afford tert-butyl N-[(35)-1-{2-tert-butyl-5-[1-(2,6-difluorophenyl)-6-oxopyridazine-3-amido]-1-methyl-1,3-benzodiazol-4-yl}pyrrolidin-3-yl]carbamate (60 mg, Yield: 44.4%) as a yellow solid.

LC-MS m/z: 622.4 (M+1)⁺.

### Step 7: N-{4-[(35)-3-aminopyrrolidin-1-yl]-2-tert-butyl-1-methyl-1,3-benzodiazol-5-yl}-1-(2,6-difluorophenyl)-6-oxopyridazine-3-carboxamide

To a solution of tert-butyl N-[(35)-1-{2-cyclopropyl-5-[1-(2,6-difluorophenyl)-6-oxopyridazine-3-amido]-1-methyl-1,3-benzodiazol-4-yl}pyrrolidin-3-yl]carbamate (35 mg, 0.052 mmol, 1.0 eq) in DCM (3 mL) was added HCl in EtOAc (0.5 mL). The reaction mixture was stirred at 25° C. for 2 hours. The reaction mixture was concentrated and washed with tert-butyl methyl ether to afford Example 98 as a yellow solid.

LC-MS m/z: 522.4 (M+1)⁺.

¹H NMR (400 MHz, CD₃OD) δ 8.39 (d, J=8.8 Hz, 1H), 8.23 (d, J=9.8 Hz, 1H), 7.80-7.63 (m, 1H), 7.43 (d, J=8.9 Hz, 1H), 7.35-7.31 (m, 3H), 4.01 (s, 3H), 4.00-3.99 (m, 1H), 3.68-3.64 (m, 1H), 3.52-3.38 (m, 3H), 2.35-2.30 (m, 1H), 2.17-2.09 (m, 1H), 1.58 (s, 9H).

| No. | Structure | Salt form | MS and ¹HNMR |
|---|---|---|---|
| Example 99 | 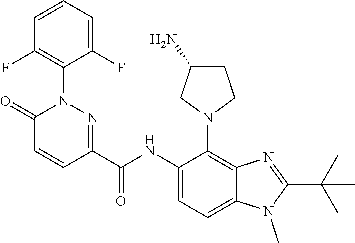 [SMILES: Cn1c(C(C)(C)C)nc2c(N3CC[C@@H](N)C3)c(NC(=O)c3ccc(=O)n(-c4c(F)cccc4F)n3)ccc21] | HCl Salt | LC-MS m/z: 522.4 (M + 1)⁺. ¹H NMR (500 MHz, CD₃OD) δ 8.22 (d, J = 9.9 Hz, 1H), 8.02 (br s, 1H), 7.74-7.61 (m, 2H), 7.37- 7.29 (m, 3H), 4.18 (s, 3H), 3.98-3.80 (m, 3H), 3.52-3.40 (m, 2H), 2.33-2.15 (m, 2H), 1.73 (s, 9H) |
| | (R)-N-(4-(3-aminopyrrolidin-1-yl)-2- | | |
| | (tert-butyl)-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 100 (isomer 1) | 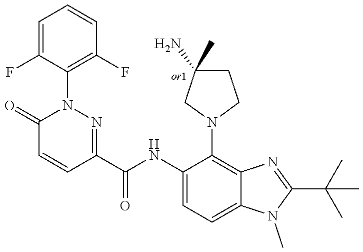 [SMILES: Cn1c(C(C)(C)C)nc2c(N3CC[C@@](C)(N)C3)c(NC(=O)c3ccc(=O)n(-c4c(F)cccc4F)n3)ccc21] | Free base | LC-MS m/z: 536.3 (M + 1)⁺. ¹H NMR (400 MHz, CD₃OD) δ ppm 8.36 (d, J = 8.82 Hz, 1 H), 8.23 (d, J = 9.89 Hz, 1 H), 7.-7- 7.75 (m, 1 H), 7.-1-7.43 (m, 4 H), 4.63 (br s, 2 H), 4.01 (s, 3 H), 3.-6-3.74 (m, 1 H), 3.-6- 3.39 (m, 3 H), 2.28 (br dd, J = 13.77, 6.62 Hz, 1 H), 1.-8- 1.98 (m, 1 H), 1.58 (s, 9 H), 1.46 (s, 3 H) |
| | ®-N-(4-(3-amino-3-methylpyrrolidin- | | |
| | 1-yl)-2-(tert-butyl)-1-methyl-1H- | | |
| | benzo 111 midazolezol-5-yl)-1-(2,6- | | |
| | difluorophenyl)-6-oxo-1,6- | | |
| | dihydropyridazine-3-carboxamide | | |
| Example 101 (isomer 2) | 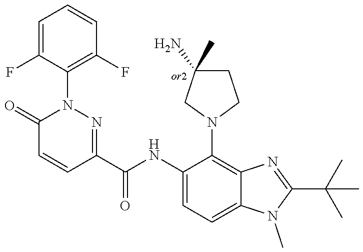 [SMILES: Cn1c(C(C)(C)C)nc2c(N3CC[C@@](C)(N)C3)c(NC(=O)c3ccc(=O)n(-c4c(F)cccc4F)n3)ccc21] | Free base | LC-MS m/z: 536.3 (M + 1)⁺. ¹H NMR (400 MHz, CD₃OD) δ ppm 8.36 (d, J = 8.82 Hz, 1 H), 8.23 (d, J = 9.78 Hz, 1 H), 7.67- 7.75 (m, 1 H), 7.31-7.44 (m, 4 H), 4.63 (br s, 2 H), 4.01 (s, 3 H), 3.69 (td, J = 10.61, 6.20 Hz, 1 H), 3.36-3.44 (m, 3 H), 2.30 (br dd, J = 13.11, 5.60 Hz, 1 H), 1.89-1.99 (m, 1 H), 1.58 (s, 9 H), 1.47 (s, 3 H) |
| | (S)-N-(4-(3-amino-3- | | |
| | methylpyrrolidin-1-yl)-2-(tert-butyl)- | | |
| | 1-methyl-1H-benzo[d] 111 midazole- | | |
| | 5-yl)-1-(2,6-difluorophenyl)-6-oxo- | | |
| | 1,6-dihydropyridazine-3-carboxamide | | |
| Example 102 | 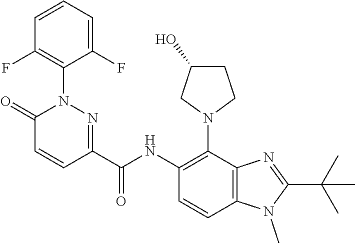 [SMILES: Cn1c(C(C)(C)C)nc2c(N3CC[C@@H](O)C3)c(NC(=O)c3ccc(=O)n(-c4c(F)cccc4F)n3)ccc21] | HCl Salt | LC-MS m/z: 523.3 (M + 1)⁺. ¹H NMR (400 MHz, CD₃OD) δ 8.38 (d, J = 8.85 Hz, 1 H), 8.23 (d, J = 9.92 Hz, 1 H), 7.-5-7.71 (m, 1 H), 7.-9-7.35 (m, 4 H), 4.35 (br s, 1 H), 3.98 (s, 3 H), 3.-3-3.49 (m, 2 H), 3.29 (br d, J = 8.85 Hz, 2 H), 2.-0-2.06 (m, 1 H), 1.79 (br d, J = 12.21 Hz, 1 H), 1.55 (s, 9 H). |
| | (R)-N-(4-(2-tert-butyl)-4-(3- | | |
| | hydroxypyrrolidin-1-yl)-1-methyl-1H- | | |
| | benzo 112 midazolezol-5-yl)-1-(2,6- | | |
| | difluorophenyl)-6-oxo-1,6- | | |
| | dihydropyridazine-3-carboxamide | | |
| Example 103 | 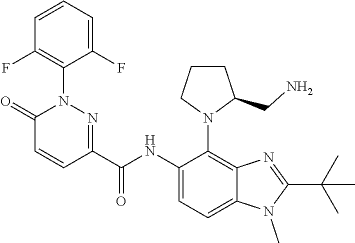 [SMILES: Cn1c(C(C)(C)C)nc2c(N3CCC[C@H]3CN)c(NC(=O)c3ccc(=O)n(-c4c(F)cccc4F)n3)ccc21] | HCOOH Salt | LC-MS m/z: 536.3 (M + 1)⁺. ¹H NMR (400 MHz, CD₃OD) δ 8.55 (s, 1H), 8.20 (d, J = 9.5 Hz, 1H), 7.-8-7.60 (m, 2H), 7.30 (br d, J = 7.2 Hz, 2H), 7.19 (br d, J = 8.7 Hz, 1H), 3.93 (s, 3H), 3.46 (br d, J = 18.8 Hz, 1H), 3.15 (br d, J = 8.6 Hz, 2H), 2.87 (br s, 2H), 2.24 (br s, 1H), 1.93 (br d, J = 6.1 Hz, 1H), 1.83 (br s, 1H), 1.69 (br s, 1H) 1.54 (s, 9H). |
| | (S)-N-(4-(2- | | |
| | (aminomethamidazoledin-1-yl)-2- | | |
| | (tert-butyl)-1-methyl-1H- | | |
| | benzoamidazolezol-5-yl)-1-(2,6- | | |
| | difluorophenyl)-6-oxo-1,6- | | |
| | dihydropyridazine-3-carboxamide | | |
| Example 104 | 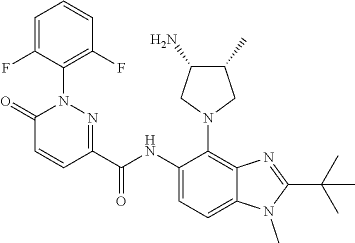 [SMILES: C[C@@H]1CN(c2c(NC(=O)c3ccc(=O)n(-c4c(F)cccc4F)n3)ccc3c2nc(C(C)(C)C)n3C)C[C@@H]1N] | HCl Salt | LC-MS m/z: 536.3 (M + 1)⁺. ¹H NMR (400 MHz, CD₃OD) δ 8.-5-8.09 (m, 2H), 7.-2-7.62 (m, 1H), 7.-4-7.48 (m, 1H), 7.- 9-7.24 (m, 3H), 4.-5-4.02 (m, 3H), 3.-1-3.74 (m, 2H), 3.-1- 3.35 (m, 3H), 2.-2-2.48 (m, 1H), 1.-1-1.58 (m, 9H), 1.-4- 1.18 (m, 3H). |
| | N-(4-((3R,4R)-3-amino-4- | | |
| | methylpyrrolidin-1-yl)-2-(tert-butyl)- | | |
| | 1-methyl-1H-benzo 112 midazolezol- | | |
| | 5-yl)-1-(2,6-difluorophenyl)-6-oxo- | | |
| | 1,6-dihydropyridazine-3-carboxamide | | |
| Example 105 | 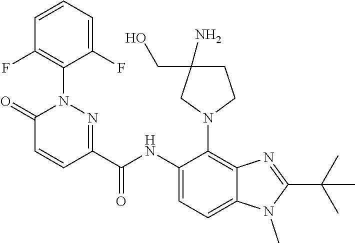 [SMILES: Cn1c(C(C)(C)C)nc2c(N3CCC(N)(CO)C3)c(NC(=O)c3ccc(=O)n(-c4c(F)cccc4F)n3)ccc21] | HCl Salt | LC-MS m/z: 552.3 (M + 1)⁺. ¹H NMR (400 MHz, CD₃OD) δ 8.36 (d, J = 8.9 Hz, 1H), 8.22 (d, J = 9.8 Hz, 1H), 7.71 (tt, J = 6.2, 8.6 Hz, 1H), 7.45 (br d, J = 7.2 Hz, 1H), 7.39-7.29 (m, 3H), 4.03 (s, 3H), 3.74-3.58 (m, 3H), 3.53-3.36 (m, 3H), 2.32 (br dd, J = 6.3, 13.9 Hz, 1H), 1.98 (ddd, J = 8.8, 10.9, 13.6 Hz, 1H), 1.61 (s, 9H). |
| | N-(4-(3-amino-3- | | |
| | (hydroxymethyl)pyrrolidin-1-yl)-2- | | |
| | (tert-butyl)-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |

### Example 106: (S)—N-(4-(3-aminopyrrolidin-1-yl)-2-methyl-1-(1-methylpiperidin-4-yl)-1H-benzo[d]imidazol-5-yl)-1-(2,6-difluorophenyl)-6-oxo-1,6-dihydropyridazine-3-carboxamide

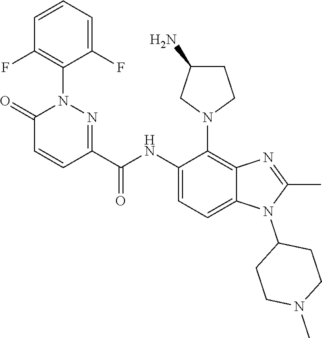 [SMILES: Cc1nc2c(N3CC[C@H](N)C3)c(NC(=O)c3ccc(=O)n(-c4c(F)cccc4F)n3)ccc2n1C1CCN(C)CC1]

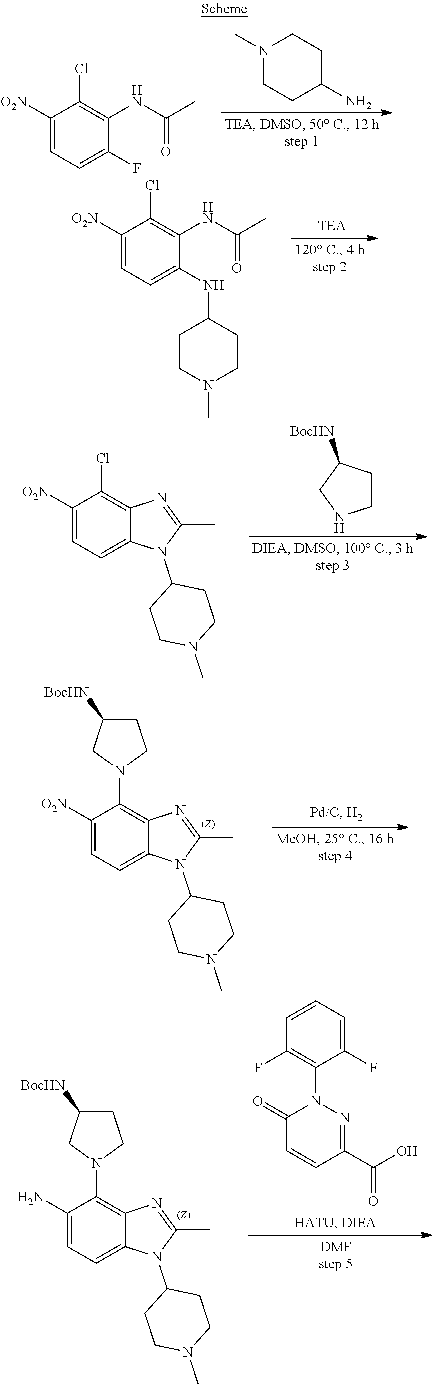

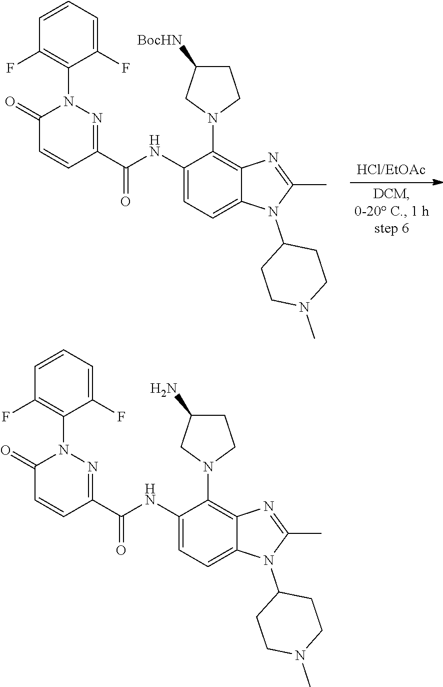 [SMILES: Cc1nc2c(N3CC[C@H](NC(=O)OC(C)(C)C)C3)c(NC(=O)c3ccc(=O)n(-c4c(F)cccc4F)n3)ccc2n1C1CCN(C)CC1 ; Cc1nc2c(N3CC[C@H](N)C3)c(NC(=O)c3ccc(=O)n(-c4c(F)cccc4F)n3)ccc2n1C1CCN(C)CC1]

### Step 1: N-(2-chloro-6-((1-methylpiperidin-4-yl)amino)-3-nitrophenyl)acetamide

A mixture of N-(2-chloro-6-fluoro-3-nitrophenyl)acetamide (200 mg, 0.86 mmol, 1 eq.), a solution of 1-methylpiperidin-4-amine (147 mg, 1.28 mmol, 1.5 eq.), and TEA (0.2 mL, 1.72 mmol, 2.0 eq.) in DMSO (3 mL) was stirred at 50° C. for 12 hours. The mixture was diluted with water (5 mL) and extracted with EtOAc (20 mL×3). The organic phase was dried over sodium sulphate and concentrated under reduced pressure to afford the crude product, which was purified by flash column chromatography to give N-(2-chloro-6-((1-methylpiperidin-4-yl)amino)-3-nitrophenyl)acetamide (300 mg, Yield: 64.1%) as black brown solid.

LC-MS m/z: 327.8 (M+1)⁺.

### Step 2: 4-chloro-2-methyl-1-(1-methylpiperidin-4-yl)-5-nitro-1H-benzo[d]imidazole

A solution of N-(2-chloro-6-((1-methylpiperidin-4-yl)amino)-3-nitrophenyl)acetamide (300 mg, 0.92 mmol, 1.0 eq), TEA (186 mg, 1.84 mmol, 2.0 eq) in DMSO (5 mL) was stirred at 100° C. for 12 hours. After cooling to room temperature, the mixture was diluted with water (50 mL) and extracted with EtOAc (50 mL×3). The combined organic layers were washed with brine (50 mL), dried over Na₂SO₄, filtered and evaporated to dryness. The crude product was purified by silica gel column chromatography (dichloromethane: methanol, from 0 to 2%) to afford 4-chloro-2-methyl-1-(1-methylpiperidin-4-yl)-5-nitro-1H-benzo[d]imidazole (120 mg, Yield: 41.1%) as a black brown solid.

LC-MS m/z: 308.8 (M+1)⁺.

### Step 3: tert-butyl (S)-(1-(2-methyl-1-(1-methylpiperidin-4-yl)-5-nitro-1H-benzo[d]imidazol-4-yl)pyrrolidin-3-yl)carbamate

A solution of 4-chloro-2-methyl-1-(1-methylpiperidin-4-yl)-5-nitro-1H-benzo[d]imidazole (120 mg, 0.88 mmol, 1.9 eq), tert-butyl N-[(35)-pyrrolidin-3-yl]carbamate (87 mg, 0.47 mmol, 1.0 eq) and DIPEA (0.15 mL, 0.78 mmol, 1.7 eq) in DMSO was stirred under nitrogen at 100° C. for 4 hrs. After cooling to room temperature, the reaction was quenched with water (5.0 mL) and extracted with EtOAc (10 mL×3). The combined organic phase was dried over Na₂SO₄ and concentrated under reduced pressure to give the crude product, which was purified by flash column chromatography (eluting with DCM/MeOH=50/1) to give tert-butyl (S)-(1-(2-methyl-1-(1-methylpiperidin-4-yl)-5-nitro-1H-benzo[d]imidazol-4-yl)pyrrolidin-3-yl)carbamate (150 mg, Yield: 80.1%) as a red solid.

LC-MS m/z: 459.9 (M+1)⁺.

### Step 4: tert-butyl (S)-(1-(5-amino-2-methyl-1-(1-methylpiperidin-4-yl)-1H-benzo[d]imidazol-4-yl)pyrrolidin-3-yl)carbamate

The mixture of Pd/C (78 mg, 10%) and tert-butyl (S)-(1-(2-methyl-1-(1-methylpiperidin-4-yl)-5-nitro-1H-benzo[d]imidazol-4-yl)pyrrolidin-3-yl)carbamate (150 mg, 0.33 mmol) in methanol (10 mL) was hydrogenated under 15 psi for 3 hours. The reaction mixture was filtered and concentrated under reduced pressure to afford tert-butyl (S)-(1-(5-amino-2-methyl-1-(1-methylpiperidin-4-yl)-1H-benzo[d]imidazole-4-yl)pyrrolidin-3-yl)carbamate (120 mg, Yield: 81.3%) as a white solid.

LC-MS m/z: 429.3 (M+1)⁺.

### Step 5: tert-butyl (S)-(1-(5-(1-(2,6-difluorophenyl)-6-oxo-1,6-dihydropyridazine-3-carboxamido)-2-methyl-1-(1-methylpiperidin-4-yl)-1H-benzo[d]imidazol-4-yl)pyrrolidin-3-yl)carbamate

To a solution of tert-butyl (S)-(1-(5-amino-2-methyl-1-(1-methylpiperidin-4-yl)-1H-benzo[d]imidazol-4-yl)pyrrolidin-3-yl)carbamate (60 mg, 0.16 mmol, 1.0 eq) and 1-(2,6-difluorophenyl)-6-oxo-1,6-dihydropyridazine-3-carboxylic acid (41 mg, 0.16 mmol, 1.0 eq) in DMF (2.0 mL) were added HATU (80 mg, 0.21 mmol, 1.3 eq) and DIPEA (0.1 mL, 0.65 mmol, 4.1 eq). The reaction mixture was stirred at 50° C. for 10 hours. After cooling to room temperature, the reaction was quenched with water (5 mL) and extracted with EtOAc (10 mL×3). The combined organic phase was washed with brine (10 mL×3), dried over anhydrous Na₂SO₄, filtrated and concentrated under reduced pressure to give crude product, which was purified by flash column chromatography (eluting with DCM/MeOH=25/1) to afford tert-butyl (S)-(1-(5-(1-(2,6-difluorophenyl)-6-oxo-1,6-dihydropyridazine-3-carboxamido)-2-methyl-1-(1-methylpiperidin-4-yl)-1H-benzo[d]imidazol-4-yl)pyrrolidin-3-yl)carbamate (80 mg, Yield: 77.7%) as a yellow solid.

LC-MS m/z: 663.7 (M+1)⁺.

### Step 6: (S)—N-(4-(3-aminopyrrolidin-1-yl)-2-methyl-1-(1-methylpiperidin-4-yl)-1H-benzo [d]imidazol-5-yl)-1-(2,6-difluorophenyl)-6-oxo-1,6-dihydropyridazine-3-carboxamide

To a solution tert-butyl (S)-(1-(5-(1-(2,6-difluorophenyl)-6-oxo-1,6-dihydro pyridazine-3-carboxamido)-2-methyl-1-(1-methy/piperidin-4-yl)-1H-benzo[d]imidazol-4-yl)pyrrolidin-3-yl)carbamate (80 mg, 0.13 mmol) in DCM (2.0 mL) was added a solution of hydrogen chloride in 1,4-dioxane (1.0 mL, 4 M). The reaction mixture was stirred at 25° C. for 1 hours. The reaction mixture was concentrated and washed with tert-butyl methyl ether to afford Example 106 (60 mg, Yield: 79.6%) as a yellow solid.

LC-MS m/z: 563.6 (M+1)⁺.

¹H NMR (400 MHz, CD₃OD) δ 8.22 (d, J=9.9 Hz, 1H), 8.17 (d, J=9.0 Hz, 1H), 7.99 (br, 1H), 7.70 (m, 1H), 7.36-7.32 (m, 3H), 5.00 (m, 1H), 3.88 (m, 1H), 3.82-3.67 (m, 4H), 3.52-3.35 (m, 4H), 3.01 (s, 3H), 2.98 (m, 5H), 2.35-2.31 (m, 2H), 2.30-2.12 (m, 2H).

### Example 107: (S)—N-(4-(3-aminopyrrolidin-1-yl)-1,2-dimethyl-1H-benzo[d]imidazol-5-yl)-1-(2,6-dichlorophenyl)-6-oxo-1,6-dihydropyridazine-3-carboxamide

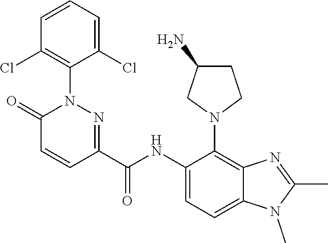 [SMILES: Cc1nc2c(N3CC[C@H](N)C3)c(NC(=O)c3ccc(=O)n(-c4c(Cl)cccc4Cl)n3)ccc2n1C]

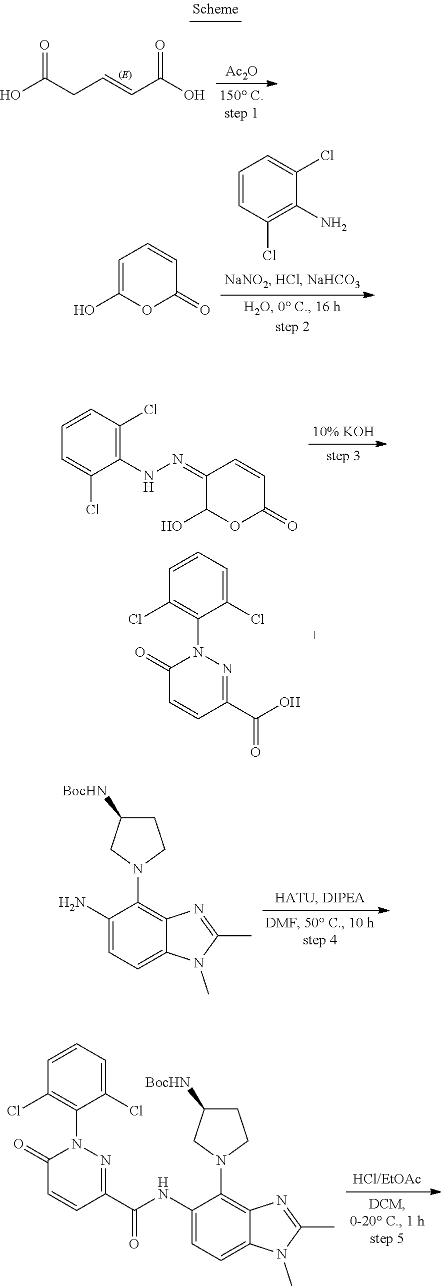

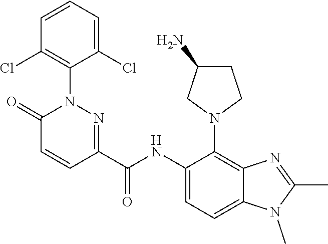 [SMILES: Cc1nc2c(N3CC[C@H](N)C3)c(NC(=O)c3ccc(=O)n(-c4c(Cl)cccc4Cl)n3)ccc2n1C]

### Step 1: 6-hydroxy-2H-pyran-2-one

A solution of (2E)-pent-2-enedioic acid (3 g, 23.1 mmol, 1 eq.) in Ac₂O (9.4 g, 92.4 mmol, 4 eq) was stirred at 150° C. for 1 hour to give a blackbrown solution. The reaction mixture was concentrated to afford 6-hydroxy-2H-pyran-2-one (3.0 g, Yield: 94.0%) as a black solid. 1H NMR (400 MHz, CDCl₃) δ 6.99 (dd, J=6.0, 4.0 Hz, 1H), 6.31 (d, J=9.8 Hz, 1H).

### Step2: (Z)-5-(2-(2,6-dichlorophenyl)hydrazineylidene)-6-hydroxy-5,6-dihydro-2H-pyran-2-one

To a solution of 2,6-dichloroaniline (5.78 g, 35.7 mol) in 3M HCl (120 mL) was added NaNO₂ (3.2 g, 46.4 mmol) in portions at 0° C. After stirring at 0° C. for 1 hour, a solution of 6-hydroxypyran-2-one (4 g, 26.8 mmol) and Na₂CO₃ (1.89 g, 17.8 mmol) in H₂O (50 mL) was added dropwise to the reaction mixture at 0° C. The reaction mixture was stirred for 16 hours to give a brown suspension. The reaction was filtered, and the filter cake was washed with H₂O and dried under vacuum to afford (Z)-5-(2-(2,6-dichlorophenyl)hydrazineylidene)-6-hydroxy-5,6-dihydro-2H-pyran-2-one (3 g, Yield: 23.5%) as a brown solid.

### Step 3: 1-(2,6-dichlorophenyl)-6-oxo-1,6-dihydropyridazine-3-carboxylic acid

To a solution of compound (Z)-5-(2-(2,6-dichlorophenyl)hydrazineylidene)-6-hydroxy-5,6-dihydro-2H-pyran-2-one (3.0 g, 10.4 mmol, 1 eq.) in H₂O (50 mL) was added KOH (2.2 g, 39.5 mmol, 5 eq.). The reaction mixture was stirred at 100° C. for 3 hours to give a blackbrown solution. The reaction was acidified with 1 N HCl to pH=2^{˜}3 and extracted with EtOAc (30 mL×3). The organic phase was washed with brine (20 mL×3), dried over Na₂SO₄, filtered and concentrated in vacuo to afford 1-(2,6-dichlorophenyl)-6-oxo-1,6-dihydropyridazine-3-carboxylic acid (0.6 g, Yield: 19.7%) as a black solid.

LC-MS m/z: 285.0 (M+1)⁺.

### Step 4: tert-butyl (S)-(1-(5-(1-(2,6-dichlorophenyl)-6-oxo-1,6-dihydropyridazine-3-carboxamido)-1,2-dimethyl-1H-benzo[d]imidazol-4-yl)pyrrolidin-3-yl)carbamate

To a solution of tert-butyl (S)-(1-(5-amino-1,2-dimethyl-1H-benzo[d]imidazole-4-yl)pyrrolidin-3-yl)carbamate (69 mg, 0.20 mmol, 1.0 eq) and 1-(2,6-dichlorophenyl)-6-oxo-1,6-dihydropyridazine-3-carboxylic acid (50 mg, 0.20 mmol, 1.0 eq) in DMF (2.0 mL) were added HATU (113 mg, 0.30 mmol, 1.5 eq) and DIPEA (0.15 ml, 0.79 mmol, 4.0 eq). The reaction mixture was stirred at 50° C. for 10 hours. After cooling to room temperature, the reaction was quenched with water (5 mL) and extracted with EtOAc (10 mL×3). The combined organic phase was washed with brine (10 mL×3), dried over anhydrous Na₂SO₄, filtrated and concentrated under reduced pressure to give crude product, which was purified by flash column chromatography (eluting with DCM/MeOH=25/1) to afford tert-butyl(S)-(1-(5-(1-(2,6-dichlorophenyl)-6-oxo-1,6-dihydropyridazine-3-carboxamido)-1,2-dimethyl-1H-benzo[d]imidazol-4-yl)pyrrolidin-3-yl)carbamate (20 mg, Yield: 15.8%) as a yellow solid.

LC-MS m/z: 613.5 (M+1)⁺.

### Step 5: N-(4-(3-aminoazetidin-1-yl)-1,2-dimethyl-1H-benzo[d]imidazol-5-yl)-2-(2-fluoro-6-methoxyphenyl)-3-oxo-2,3-dihydropyridazine-4-carboxamide

To a solution tert-butyl (S)-(1-(5-(1-(2,6-dichlorophenyl)-6-oxo-1,6-dihydropyridazine-3-carboxamido)-1,2-dimethyl-1H-benzo[d]imidazol-4-yl)pyrrolidin-3-yl)carbamate (20 mg, 0.03 mmol) in DCM (2.0 mL) was added a solution of hydrogen chloride in 1,4-dioxane (1.0 mL, 4 M). The reaction mixture was stirred at 25° C. for 1 hours. The reaction mixture was concentrated and washed with tert-butyl methyl ether to afford Example 107 (10 mg, Yield: 23.5%) as a yellow solid.

LC-MS m/z: 513.5 (M+1)⁺.

¹H NMR: (400 MHz, CD₃OD) δ 8.15 (d, J=9.9 Hz, 1H), 8.09 (d, J=8.9 Hz, 1H), 7.62 (d, J=8.3 Hz, 2H), 7.56-7.48 (m, 2H), 7.24 (d, J=9.9 Hz, 1H), 3.84 (s, 3H), 3.66 (m, 2H), 3.61-3.52 (m, 1H), 3.39-3.30 (m, 1H), 3.19-3.10 (m, 1H), 2.78 (s, 3H), 2.06-1.97 (m, 2H).

| No. | Structure | Salt form | MS and ¹HNMR |
|---|---|---|---|
| Example 108 | 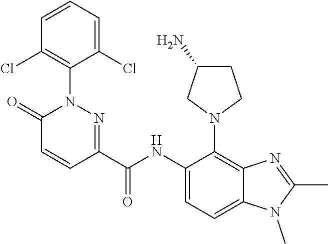 [SMILES: Cc1nc2c(N3CC[C@@H](N)C3)c(NC(=O)c3ccc(=O)n(-c4c(Cl)cccc4Cl)n3)ccc2n1C] | HCl Salt | LC-MS m/z: 513.5 (M + 1)⁺. ¹H NMR (400 MHz, CD₃OD) δ 8.26 (d, 1 H), 8.26 (d, 1 H), 7.62 (d, 2 H), 7.37 (d, 2 H), 7.37 (d, 1 H), 3.96 (s, 3 H), 3.68-3.79 (m, 3 H), 3.42-3.45 (m, 2 H), 2.88 (s, 3 H), 2.14 (m, 2 H). |
| | (R)-N-(4-(3-aminopyrrolidin-1-yl)- | | |
| | 1,2-dimethyl-1H-benzo[d]imidazol- | | |
| | 5-yl)-1-(2,6-dichlorophenyl)-6-oxo- | | |
| | 1,6-dihydropyridazine-3- | | |
| | carboxamide | | |

### Example 109: (R)—N-(4-(3-aminopyrrolidin-1-yl)-2-isopropyl-1-methyl-1H-benzo[d]imidazol-5-yl)-1-(2,6-difluorophenyl)-6-oxo-1,6-dihydropyridazine-3-carboxamide

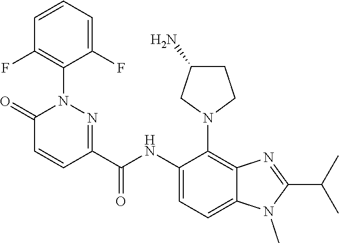 [SMILES: CC(C)c1nc2c(N3CC[C@@H](N)C3)c(NC(=O)c3ccc(=O)n(-c4c(F)cccc4F)n3)ccc2n1C]

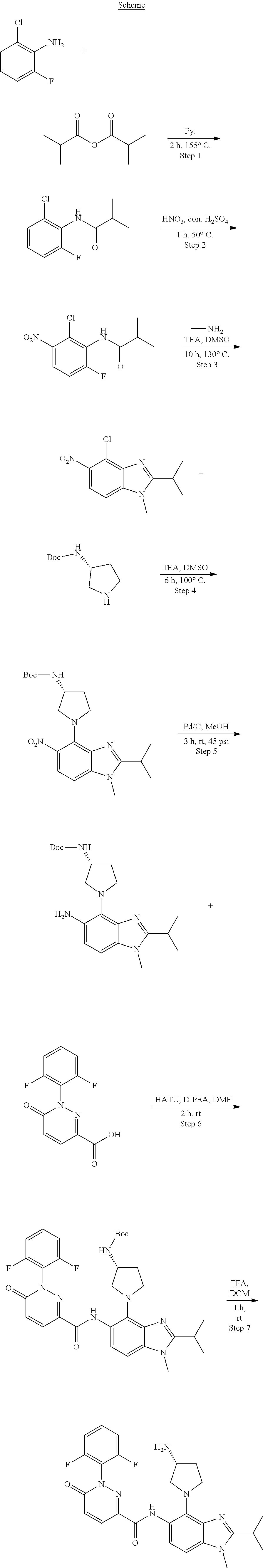

### Step 1: N-(2-chloro-6-fluorophenyl)isobutyramide

A solution of 2-chloro-6-fluoroaniline (6.5 g, 44.65 mmol, 1 eq.) in isobutyric anhydride (8.48 g, 53.59 mmol, 1.2 eq.) and Py. (4.24 g, 53.59 mmol, 1.2 eq.). The reaction was stirred for 2 hours at 155° C. by microwave to give a light brown solution. LCMS showed the reaction the starting material was consumed up and one major peak with desired mass was detected. Two reactions were conducted in parallel and combined for the workup. The reactions were poured into water and a lot of solid was precipitated. Filtered and the filter cake was dried by oil pump to afford N-(2-chloro-6-fluorophenyl)isobutyramide (19.8 g, Yield: 103%) as a light brown solid.

LC-MS m/z: 216.1 (M+1)⁺

### Step 2: N-(2-chloro-6-fluoro-3-nitrophenyl)isobutyramide

To a solution of N-(2-chloro-6-fluorophenyl)isobutyramide (89.31 mmol, 1 eq.) in 98% H₂SO₄ (40 ml) was added 66% HNO₃ (7.34 mL, 107.17 mmol, 1.2 eq.) dropwise at 0° C. The ice bath was removed and the reaction was stirred at rt for 30 mins. Then the reaction was warmed to 50° C. and stirred for another 1 hour to give a black brown solution. LCMS showed the starting material was consumed up and one major peak with desired mass was detected. The mixture was then poured into ice water and basified with Na₂CO₃ to pH=2^{˜}3 and then extracted with EA (50 mL×3). The combined organic layers were washed with brine, dried over Na₂SO₄, filtered and concentrated to dryness. The residue was purified by flash column (Silica column, eluted with 5% to 15% EA in PE) to afford N-(2-chloro-6-fluoro-3-nitrophenyl)isobutyramide (9.5 g, Yield: 41%) as a brown solid.

LC-MS m/z: 261.1 (M+1)⁺

### Step 3: 4-chloro-2-isopropyl-1-methyl-5-nitro-1H-benzo[d]imidazole

To a solution of N-(2-chloro-6-fluoro-3-nitrophenyl)isobutyramide (9.5 g, 36.45 mmol, 1 eq.) in DMSO (100 ml) was added TEA (7.38 g, 72.89 mmol, 2 eq.) and 33% methanamine in EtOH (7.77 mL, 54.67 mmol, 1.5 eq.). The reaction was stirred at rt for 1 hour and then warmed to 130° C. and stirred for another 10 hours to give a black brown solution. LCMS showed the starting material was consumed up and two new peaks was detected. The mixture was diluted with H₂O and extracted with EA (80 mL×3). The combined organic layers were washed with brine, dried over Na₂SO₄, filtered and concentrated to dryness. The residue was purified by flash column (Silica column, eluted with 10% to 20% EA in PE) to afford 4-chloro-2-isopropyl-1-methyl-5-nitro-1H-benzo[d]imidazole (7.5 g, Yield: 81.1%) as a brown solid.

LC-MS m/z: 254.1 (M+1)⁺

### Step 4: tert-butyl (R)-(1-(2-isopropyl-1-methyl-5-nitro-1H-benzo[d]imidazol-4-yl)pyrrolidin-3-yl)carbamate

To a solution of 4-chloro-2-isopropyl-1-methyl-5-nitro-1H-benzo[d]imidazole (3 g, 11.83 mmol, 1 eq.) in DMSO (30 ml) was added TEA (3.59 g, 35.48 mmol, 3 eq.) and tert-butyl (R)-pyrrolidin-3-ylcarbamate (2.2 g, 11.83 mmol, 1 eq.). The reaction was stirred for 6 hours at 100° C. to give a black brown solution. LCMS showed the starting material was consumed up and one major peak with desired mass was detected. The reaction was diluted with H₂O and extracted with EA (30 mL×3). The combined organic layers were washed with brine, dried over Na₂SO₄, filtered and concentrated to afford tert-butyl (R)-(1-(2-isopropyl-1-methyl-5-nitro-1H-benzo[d]imidazol-4-yl)pyrrolidin-3-yl)carbamate (4.7 g, Yield: 98.5%) as a brown solid.

LC-MS m/z: 404.3 (M+1)⁺

### Step 5: tert-butyl (R)-(1-(5-amino-2-isopropyl-1-methyl-1H-benzo[d]imidazol-4-yl)pyrrolidin-3-yl)carbamate

To a solution of tert-butyl (R)-(1-(2-isopropyl-1-methyl-5-nitro-1H-benzo[d]imidazole-4-yl)pyrrolidin-3-yl)carbamate (4.7 g, 11.65 mmol, 1 eq.) in MeOH (50 mL) was added Pd/C (0.4 g) under N₂. The reaction was degassed with H₂ for several times and stirred for 3 hours at rt under 45 psi to give a black brown suspension. LCMS showed the starting material was consumed up and one major peak with desired mass was detected. The reaction was filtered through a pad of Celite and the filter cake was washed with MeOH. The filtrate was concentrated to afford tert-butyl (R)-(1-(5-amino-2-isopropyl-1-methyl-1H-benzo[d]imidazol-4-yl)pyrrolidin-3-yl)carbamate (4.2 g, Yield: 97.7%) as brown gum.

LC-MS m/z: 374.3 (M+1)⁺

### Step 6: tert-butyl (R)-(1-(5-(1-(2,6-difluorophenyl)-6-oxo-1,6-dihydropyridazine-3-carboxamido)-2-isopropyl-1-methyl-1H-benzo[d]imidazol-4-yl)pyrrolidin-3-yl)carbamate

To a solution of 1-(2,6-difluorophenyl)-6-oxo-1,6-dihydropyridazine-3-carboxylic acid (1.3 g, 5.16 mmol, 1 eq.) and tert-butyl (R)-(1-(5-amino-2-isopropyl-1-methyl-1H-benzo[d]imidazol-4-yl)pyrrolidin-3-yl)carbamate (2.12 g, 5.67 mmol, 1.1 eq.) in DMF (20 mL) was added HATU (2.55 g, 6.7 mmol, 1.3 eq.) and DIEA (2 g, 15.47 mmol, 3 eq.). The reaction was stirred for 2 hours at rt to give a black brown solution. LCMS showed the starting material was consumed up and one major peak with desired mass was detected. The reaction was treated with water and the precipitated solid was collected by filtration. The filtrate cake was washed with water and dried by oil pump to dryness. The product was triturated with MTBE/hexane afford tert-butyl(R)-(1-(5-(1-(2,6-difluorophenyl)-6-oxo-1,6-dihydropyridazine-3-carboxamido)-2-isopropyl-1-methyl-1H-benzo[d]imidazol-4-yl)pyrrolidin-3-yl)carbamate (2.7 g, Yield: 86%) as a yellow solid.

LC-MS m/z: 608.3 (M+1)⁺

### Step 7: (R)—N-(4-(3-aminopyrrolidin-1-yl)-2-isopropyl-1-methyl-1H-benzo[d]imidazol-5-yl)-1-(2,6-difluorophenyl)-6-oxo-1,6-dihydropyridazine-3-carboxamide

To a solution of tert-butyl (R)-(1-(5-(1-(2,6-difluorophenyl)-6-oxo-1,6-dihydropyridazine-3-carboxamido)-2-isopropyl-1-methyl-1H-benzo[d]imidazol-4-yl)pyrrolidin-3-yl)carbamate (2.7 g, 4.44 mmol, 1 eq.) in DCM (10 mL) was added TFA (5 mL). The reaction was stirred for 1 hour at rt to give a brown solution. LCMS showed the starting material was consumed up and one major peak with desired mass was detected. The reaction was basified with NaHCO₃ aq. to pH=9 and extracted with DCM (30 mL×3). The combined organic layers were washed with brine, dried over Na₂SO₄, filtered and concentrated to dryness. The afford product was triturated with EA/MTBE, filtered and the filter cake was lyophilized with 3 M HCl aq. to afford Example 109 (1489.32 mg) as a yellow solid.

LC-MS m/z: 508.3 (M+1)⁺

¹H NMR (400 MHz, CD₃OD) δ 8.21 (d, J=9.8 Hz, 1H), 8.13 (d, J=8.9 Hz, 1H), 7.68 (t, J=8.0 Hz, 2H), 7.31 (d, J=9.7 Hz, 3H), 4.04 (s, 3H), 3.94-3.78 (m, 2H), 3.73 (t, J=8.8 Hz, 2H), 3.47 (d, J=10.2 Hz, 1H), 2.21 (dd, J=15.5, 7.8 Hz, 2H), 1.63 (dd, J=10.7, 7.0 Hz, 6H).

| No. | Structure | Salt form | MS and ¹HNMR |
|---|---|---|---|
| Example 110 | 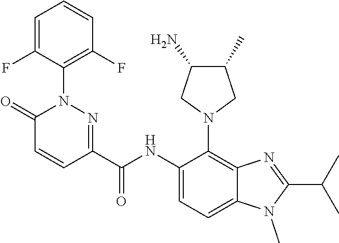 [SMILES: CC(C)c1nc2c(N3C[C@@H](C)[C@@H](N)C3)c(NC(=O)c3ccc(=O)n(-c4c(F)cccc4F)n3)ccc2n1C] | HCl salt | LC-MS m/z: 522.3 (M + 1)⁺ ¹H NMR (400 MHz, d₆- DMSO + D₂O) δ 8.08 (d, J = 9.8 Hz, 1H), 7.94 (d, J = 8.8 Hz, 1H), 7.71-7.61 (m, 1H), 7.42-7.33 (m, 3H), 7.30 (d, J = 9.9 Hz, 1H), 3.74 (s, 3H), 3.72-3.61 (m, 2H), 3.33 (p, J = 6.9 Hz, 1H), 3.27- 3.19 (m, 2H), 3.11 (t, J = 9.7 Hz, 1H), 2.66 (s, 2H), 2.33 (s, 1H), 1.30 (dd, J = 6.9, 2.4 Hz, 6H), 1.02 (d, J = 7.0 Hz, 3H). |
| | N-(4-((3R,4R)-3-amino-4- | | |
| | methylpyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 111 | 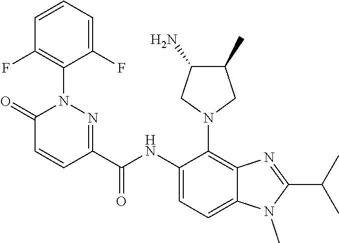 [SMILES: CC(C)c1nc2c(N3C[C@H](C)[C@@H](N)C3)c(NC(=O)c3ccc(=O)n(-c4c(F)cccc4F)n3)ccc2n1C] | HCl salt | LC-MS m/z: 522.3 (M + 1)⁺ ¹H NMR (400 MHz, CD₃OD) δ 8.29 (dd, J = 16.4, 9.4 Hz, 2H), 7.69 (tt, J = 8.5, 6.2 Hz, 1H), 7.49 (d, J = 8.8 Hz, 1H), 7.41- 7.26 (m, 3H), 3.87 (s, 3H), 3.75 (dd, J = 12.4, 5.1 Hz, 1H), 3.65 (dd, J = 9.6, 6.1 Hz, 1H), 3.56 (d, J = 5.0 Hz, 1H), 3.50-3.39 (m, 1H), 3.35 (s, 1H), 2.95 (dd, J = 9.6, 2.1 Hz, 1H), 2.63-2.51 (m, 1H), 1.42 (dd, J = 6.9, 1.9 Hz, 6H), 0.86 (d, J = 7.0 Hz, 3H). |
| | N-(4-((3R,4S)-3-amino-4- | | |
| | methylpyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 112 | 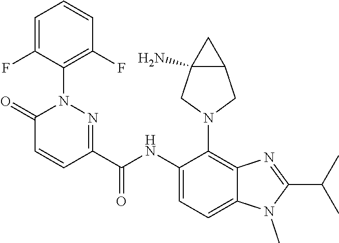 [SMILES: CC(C)c1nc2c(N3CC4C[C@]4(N)C3)c(NC(=O)c3ccc(=O)n(-c4c(F)cccc4F)n3)ccc2n1C] | HCl salt | LC-MS m/z: 520.3 (M + 1)⁺ ¹H NMR (400 MHz, CD₃OD) δ 10.23 (s, 1H), 8.81 (d, J = 9.1 Hz, 1H), 8.26 (d, J = 9.9 Hz, 1H), 7.88- 7.64 (m, 2H), 7.35 (dt, J = 16.4, 8.8 Hz, 3H), 4.10 (s, 1H), 4.07 (s, 3H), 3.88 (dd, J = 8.5, 4.0 Hz, 1H), 3.76 (p, J = 6.9 Hz, 1H), 3.06 (d, J = 8.5 Hz, 1H), 1.98 (dt, J = 9.3, 4.4 Hz, 1H), 1.66 (dd, J = 7.3, 3.0 Hz, 6H), 1.62-1.26 (m, 1H), 0.87 (t, J = 5.8 Hz, 1H), 0.70- 0.55 (m, 1H). |
| | N-(4-((1R)-1-amino-3- | | |
| | azabicyclo[3.1.0]hexan-3-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 113 | 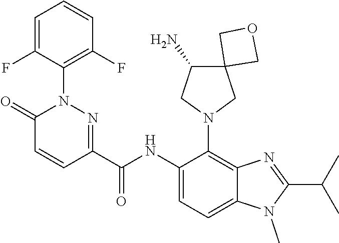 [SMILES: CC(C)c1nc2c(N3C[C@H](N)C4(COC4)C3)c(NC(=O)c3ccc(=O)n(-c4c(F)cccc4F)n3)ccc2n1C] | HCl salt | LC-MS m/z: 550.3 (M + H)⁺ ¹H NMR (400 MHz, CD₃OD) δ 8.47 (d, J = 8.9 Hz, 1H), 8.25 (d, J = 9.9 Hz, 1H), 7.66 (tt, J = 8.6, 6.2 Hz, 1H), 7.44 (d, J = 8.9 Hz, 1H), 7.36-7.25 (m, 3H), 4.92 (d, J = 7.2 Hz, 1H), 4.25 (d, J = 6.0 Hz, 1H), 4.14 (d, J = 4.4 Hz, 1H), 3.97 (d, J = 6.0 Hz, 1H), 3.83 (s, 3H), 3.67-3.55 (m, 3H), 3.38 (td, J = 13.4, 6.6 Hz, 2H), 1.40 (dd, J = 9.0, 6.8 Hz, 6H). |
| | (R)-N-(4-(8-amino-2-oxa-6- | | |
| | azaspiro[3.4]octan-6-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 114 | 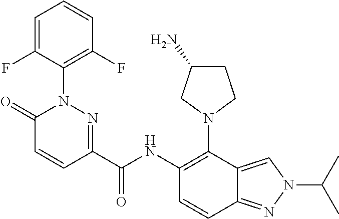 [SMILES: CC(C)n1cc2c(N3CC[C@@H](N)C3)c(NC(=O)c3ccc(=O)n(-c4c(F)cccc4F)n3)ccc2n1] | TFA salt | LC-MS m/z: 494.3 (M + 1)⁺ ¹H NMR (500 MHz, CD₃OD) δ 8.46 (s, 1H), 8.27-8.19 (m, 1H), 7.78 (br d, J = 9.0 Hz, 1H), 7.74-7.64 (m, 1H), 7.38-7.28 (m, 4H), 4.82 (br d, J = 6.7 Hz, 1H), 3.75 (br d, J = 7.2 Hz, 2H), 3.66 (q, J = 7.6 Hz, 1H), 3.42- 3.36 (m, 2H), 2.32-2.23 (m, 1H), 1.97-1.88 (m, 1H), 1.65 (br d, J = 6.6 Hz, 6H). |
| | (R)-N-(4-(3-aminopyrrolidin-1-yl)- | | |
| | 2-isopropyl-2H-indazol-5-yl)-1- | | |
| | (2,6-difluorophenyl)-6-oxo-1,6- | | |
| | dihydropyridazine-3-carboxamide | | |
| Example 115 | 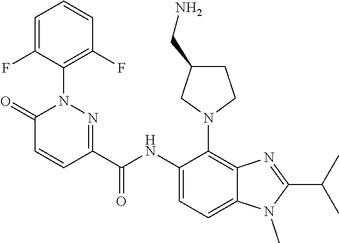 [SMILES: CC(C)c1nc2c(N3CC[C@H](CN)C3)c(NC(=O)c3ccc(=O)n(-c4c(F)cccc4F)n3)ccc2n1C] | HCl salt | LC-MS m/z: 522.2 (M + 1)⁺ ¹H NMR (500 MHz, CD₃OD) δ 8.23 (d, J = 9.9 Hz, 2H), 7.74- 7.64 (m, 1H), 7.50 (br s, 1H), 7.36-7.29 (m, 3H), 3.93 (br s, 3H), 3.65 (br s, 1H), 3.43 (br d, J = 17.9 Hz, 4H), 2.93 (br d, J = 6.7 Hz, 2H), 2.66 (br s, 1H), 2.16 (br s, 1H), 1.66 (br s, 1H), 1.50 (br s, 6H). |
| | (R)-N-(4-(3- | | |
| | (aminomethyl)pyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 116 | 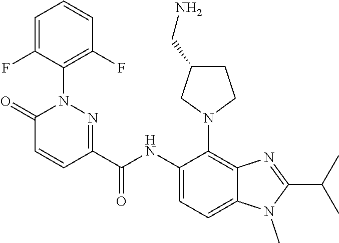 [SMILES: CC(C)c1nc2c(N3CC[C@@H](CN)C3)c(NC(=O)c3ccc(=O)n(-c4c(F)cccc4F)n3)ccc2n1C] | HCl salt | LC-MS m/z: 522.2 (M + 1)⁺ ¹H NMR (500 MHz, CD₃OD) δ 8.45-8.21 (m, 2H), 7.81-7.68 (m, 2H), 7.46-7.29 (m, 3H), 4.07 (s, 3H), 3.76 (td, J = 6.8, 13.8 Hz, 1H), 3.71-3.59 (m, 1H), 3.57-3.43 (m, 2H), 2.94 (br d, J = 6.4 Hz, 2H), 2.77 (br s, 1H), 2.30-2.18 (m, 1H), 1.76-1.67 (m, 1H), 1.66-1.57 (m, 6H). |
| | (S)-N-(4-(3- | | |
| | (aminomethyl)pyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 117 | 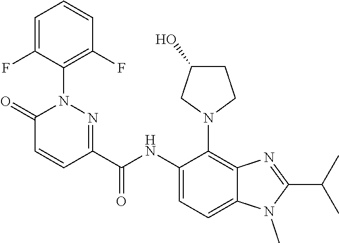 [SMILES: CC(C)c1nc2c(N3CC[C@@H](O)C3)c(NC(=O)c3ccc(=O)n(-c4c(F)cccc4F)n3)ccc2n1C] | HCl salt | LC-MS m/z: 509.2 (M + 1)⁺ ¹H NMR (500 MHz, CD₃OD) δ 8.63 (br d, J = 8.9 Hz, 1H), 8.22 (d, J = 9.9 Hz, 1H), 7.78-7.63 (m, 2H), 7.35-7.26 (m, 3H), 4.61 (br s, 1H), 4.04 (s, 3H), 3.73- 3.62 (m, 1H), 3.57-3.47 (m, 1H), 3.43 (br t, J = 8.5 Hz, 1H), 3.36-3.32 (m, 1H), 3.25-3.14 (m, 1H), 2.17 (br dd, J = 5.4, 13.2 Hz, 1H), 1.98-1.85 (m, 1H), 1.50 (dd, J = 2.1, 6.9 Hz, 6H). |
| | (R)-1-(2,6-difluorophenyl)-N-(4-(3- | | |
| | hydroxypyrrolidin-1-yl)-2- | | |
| | isopropyl-1-methyl-1H- | | |
| | benzo[d]imidazol-5-yl)-6-oxo-1,6- | | |
| | dihydropyridazine-3-carboxamide | | |
| Example 118 | 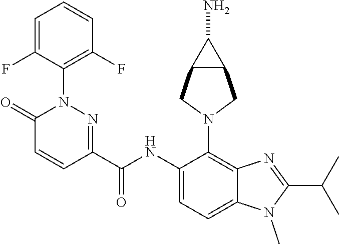 | TFA salt | LC-MS m/z: 520.2 (M + 1)⁺ 1H NMR (400 MHz, CD₃OD) δ 8.25-8.19 (m, 1H), 7.93-7.88 (m, 1H), 7.73-7.63 (m, 1H), 7.38-7.27 (m, 3H), 7.24-7.19 (m, 1H), 3.95-3.86 (m, 2H), 3.79-3.73 (m, 3H), 3.52-3.44 (m, 2H), 3.28 (br s, 1H), 2.82- 2.67 (m, 1H), 1.97-1.87 (m, 2H), 1.43-1.39 (m, 6H). |
| | N-(4-((1R,5S,6s)-6-amino-3- | | |
| | azabicyclo[3.1.0]hexan-3-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 119 | 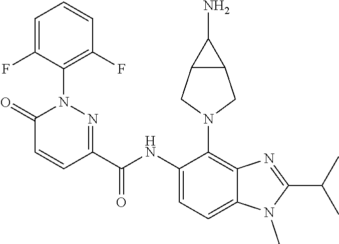 [SMILES: CC(C)c1nc2c(N3CC4C(N)C4C3)c(NC(=O)c3ccc(=O)n(-c4c(F)cccc4F)n3)ccc2n1C] | TFA salt | LC-MS m/z: 520.2 (M + 1)⁺ ¹H NMR (400 MHz, CD₃OD) δ 8.24-8.17 (m, 1H), 7.88-7.79 (m, 1H), 7.70-7.59 (m, 1H), 7.35-7.25 (m, 4H), 3.82-3.76 (m, 5H), 3.53-3.46 (m, 2H), 3.38-3.34 (m, 1H), 2.90-2.82 (m, 1H), 1.95-1.89 (m, 2H), 1.42-1.38 (m, 6H). |
| | N-(4-((1R,5S,6r)-6-amino-3- | | |
| | azabicyclo[3.1.0]hexan-3-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 120 | 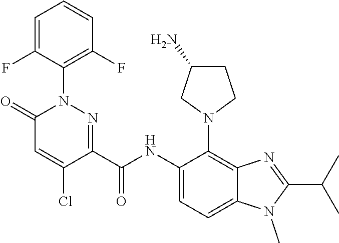 [SMILES: CC(C)c1nc2c(N3CC[C@@H](N)C3)c(NC(=O)c3nn(-c4c(F)cccc4F)c(=O)cc3Cl)ccc2n1C] | TFA salt | LC-MS m/z: 542.2 (M + 1)⁺ ¹H NMR (500 MHz, CD₃OD) δ 8.05-7.85 (m, 1H), 7.72-7.60 (m, 2H), 7.55-7.46 (m, 1H), 7.35-7.25 (m, 2H), 4.12-3.95 (m, 3H), 3.91-3.63 (m, 4H), 3.47-3.34 (m, 2H), 2.37-2.15 (m, 2H), 1.64-1.54 (m, 6H). |
| | (R)-N-(4-(3-aminopyrrolidin-1-yl)- | | |
| | 2-isopropyl-1-methyl-1H- | | |
| | benzo[d]imidazol-5-yl)-4-chloro-1- | | |
| | (2,6-difluorophenyl)-6-oxo-1,6- | | |
| | dihydropyridazine-3-carboxamide | | |
| Example 121 | 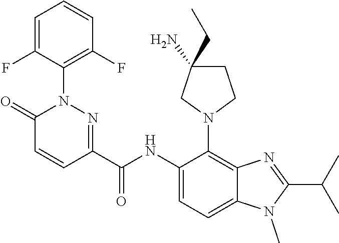 [SMILES: CC[C@@]1(N)CCN(c2c(NC(=O)c3ccc(=O)n(-c4c(F)cccc4F)n3)ccc3c2nc(C(C)C)n3C)C1] | Free base | LC-MS m/z: 536.3 (M + 1)⁺ ¹H NMR (400 MHz, CD₃OD) δ 8.37-8.28 (m, 1H), 8.24-8.19 (m, 1H), 7.74-7.65 (m, 1H), 7.49-7.39 (m, 1H), 7.36-7.29 (m, 3H), 3.86 (br s, 3H), 3.69- 3.56 (m, 1H), 3.35 (br s, 4H), 2.35-2.25 (m, 1H), 2.06-1.90 (m, 2H), 1.73-1.66 (m, 1H), 1.51-1.40 (m, 6H), 1.12-0.99 (m, 3H). |
| | (R)-N-(4-(3-amino-3- | | |
| | ethylpyrrolidin-1-yl)-2-isopropyl- | | |
| | 1-methyl-1H-benzo[d]imidazol-5- | | |
| | yl)-1-(2,6-difluorophenyl)-6-oxo- | | |
| | 1,6-dihydropyridazine-3- | | |
| | carboxamide | | |
| Example 122 | 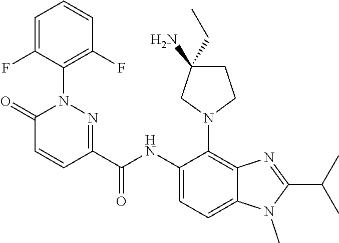 [SMILES: CC[C@]1(N)CCN(c2c(NC(=O)c3ccc(=O)n(-c4c(F)cccc4F)n3)ccc3c2nc(C(C)C)n3C)C1] | Free base | LC-MS m/z: 536.3 (M + 1)⁺ ¹H NMR (400 MHz, CD₃OD) δ 8.37-8.1 (m, 2H), 7.74-7.39 (m, 2H), 7.36-7.29 (m, 3H), 3.86 (br s, 3H), 3.69-3.56 (m, 1H), 3.35 (br s, 4H), 2.35-2.25 (m, 1H), 2.06-1.90 (m, 2H), 1.73-1.40 (m, 8H), 1.12-0.99 (m, 3H). |
| | (S)-N-(4-(3-amino-3- | | |
| | ethylpyrrolidin-1-yl)-2-isopropyl- | | |
| | 1-methyl-1H-benzo[d]imidazol-5- | | |
| | yl)-1-(2,6-difluorophenyl)-6-oxo- | | |
| | 1,6-dihydropyridazine-3- | | |
| | carboxamide | | |
| Example 123 | 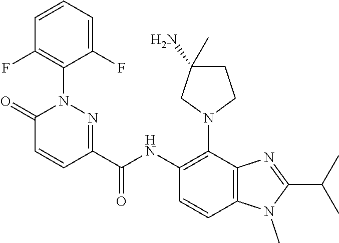 [SMILES: CC(C)c1nc2c(N3CC[C@@](C)(N)C3)c(NC(=O)c3ccc(=O)n(-c4c(F)cccc4F)n3)ccc2n1C] | HCOOH salt | LC-MS m/z: 522.3 (M + 1)⁺. ¹H NMR (400 MHz, CD₃OD) δ 8.56 (s, 1H), 8.33 (d, J = 8.8 Hz, 1H), 8.18 (d, J = 9.8 Hz, 1H), 7.71 (tt, J = 8.6, 6.2 Hz, 1H), 7.48- 7.20 (m, 4H), 3.85 (s, 3H), 3.66 (ddd, J = 11.4, 9.8, 6.4 Hz, 1H), 3.46-3.35 (m, 4H), 2.35 (dd, J = 13.8, 6.2 Hz, 1H), 1.95 (ddd, J = 13.7, 11.4, 8.3 Hz, 1H), 1.50 (s, 3H), 1.43 (dd, J = 6.9, 3.2 Hz, 6H). |
| | (R)-N-(4-(3-amino-3- | | |
| | methylpyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 124 (isomer 1) | 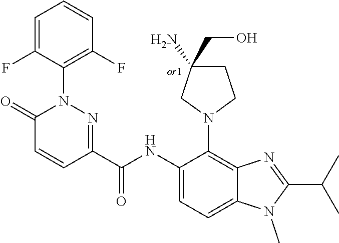 [SMILES: CC(C)c1nc2c(N3CC[C@](N)(CO)C3)c(NC(=O)c3ccc(=O)n(-c4c(F)cccc4F)n3)ccc2n1C] | Free base | LC-MS m/z: 538.2 (M + 1)⁺ ¹H NMR (400 MHz, CD₃OD) δ 8.45 (d, J = 8.8 Hz, 1H), 8.24 (d, J = 9.9 Hz, 1H), 7.80-7.61 (m, 2H), 7.40-7.23 (m, 3H), 4.02 (s, 3H), 3.96-3.85 (m, 1H), 3.76- 3.63 (m, 1H), 3.69 (s, 1H), 3.46 (d, J = 10.4 Hz, 3H), 2.26 (dd, J = 6.6, 13.6 Hz, 1H), 1.80 (s, 1H), 1.62 (s, 6H). |
| | rel-(R)-N-(4-(3-amino-3- | | |
| | (hydroxymethyl)pyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 125 (isomer 2) | 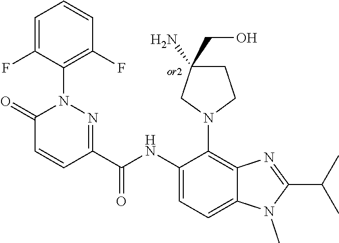 [SMILES: CC(C)c1nc2c(N3CC[C@](N)(CO)C3)c(NC(=O)c3ccc(=O)n(-c4c(F)cccc4F)n3)ccc2n1C] | Free base | LC-MS m/z: 538.2 (M + 1)⁺ ¹H NMR (400 MHz, CD₃OD) δ 8.33 (d, J = 8.9 Hz, 1H), 8.21 (d, J = 9.9 Hz, 1H), 7.68 (quin, J = 7.2 Hz, 1H), 7.41 (d, J = 8.7 Hz, 1H), 7.37-7.27 (m, 3H), 3.84 (s, 3H), 3.70-3.55 (m, 3H), 3.45 (s, 2H), 3.39 (d, J = 6.3 Hz, 1H), 2.29 (dd, J = 6.2, 13.8 Hz, 1H), 2.03-1.92 (m, 1H), 1.43 (dd, J = 3.8, 6.4 Hz, 6H). |
| | rel- | | |
| | (R)-N-(4-(3-amino-3- | | |
| | (hydroxymethyl)pyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 126 | 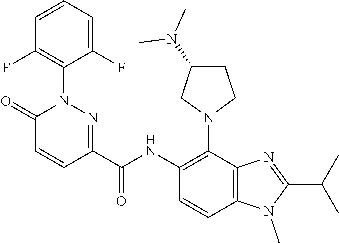 [SMILES: CC(C)c1nc2c(N3CC[C@@H](N(C)C)C3)c(NC(=O)c3ccc(=O)n(-c4c(F)cccc4F)n3)ccc2n1C] | HCOOH salt | LC-MS m/z: 536.0 (M + 1)⁺ ¹H NMR (400 MHz, CD₃OD) δ 8.29 (d, J = 8.7 Hz, 1H), 8.23 (d, J = 9.9 Hz, 1H), 7.71 (tt, J = 8.6, 6.2 Hz, 1H), 7.41-7.32 (m, 2H), 7.29 (d, J = 9.8 Hz, 1H), 7.24 (d, J = 8.8 Hz, 1H), 3.77 (s, 3H), 3.66- 3.53 (m, 3H), 2.92 (p, J = 7.5 Hz, 1H), 2.22 (s, 6H), 2.01-1.88 (m, 1H), 1.88-1.75 (m, 1H), 1.43-1.37 (m, 6H). |
| | (R)-1-(2,6-difluorophenyl)-N-(4-(3- | | |
| | (dimethylamino)pyrrolidin-1-yl)-2- | | |
| | isopropyl-1-methyl-1H- | | |
| | benzo[d]imidazol-5-yl)-6-oxo-1,6- | | |
| | dihydropyridazine-3-carboxamide | | |
| Example 127 | 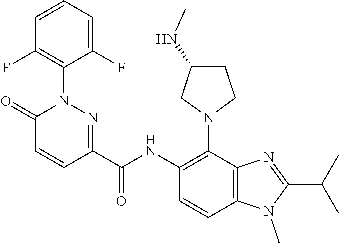 [SMILES: CN[C@@H]1CCN(c2c(NC(=O)c3ccc(=O)n(-c4c(F)cccc4F)n3)ccc3c2nc(C(C)C)n3C)C1] | HCOOH salt | LC-MS m/z: 522.2.0 (M + 1)⁺ ¹H NMR (400 MHz, CD₃OD) δ 8.57 (s, 1H), 8.32 (d, J = 8.8 Hz, 1H), 8.23 (d, J = 9.8 Hz, 1H), 7.69 (tt, J = 8.6, 6.2 Hz, 1H), 7.40 (d, J = 8.8 Hz, 1H), 7.33 (t, J = 9.2 Hz, 3H), 4.58 (s, 1H), 3.85 (s, 3H), 3.77 (s, 1H), 3.66 (dd, J = 12.5, 5.5 Hz, 1H), 3.59-3.48 (m, 2H), 3.42 (p, J = 6.9 Hz, 1H), 3.36 (d, J = 3.2 Hz, 1H), 2.77 (s, 3H), 2.36-2.24 (m, 1H), 2.15 (dddd, J = 14.0, 9.8, 8.1, 6.1 Hz, 1H), 1.43 (dd, J = 6.8, 3.9 Hz, 6H). |
| | (R)-1-(2,6-difluorophenyl)-N-(2- | | |
| | isopropyl-1-methyl-4-(3- | | |
| | (methylamino)pyrrolidin-1-yl)-1H- | | |
| | benzo[d]imidazol-5-yl)-6-oxo-1,6- | | |
| | dihydropyridazine-3-carboxamide | | |
| Example 128 | 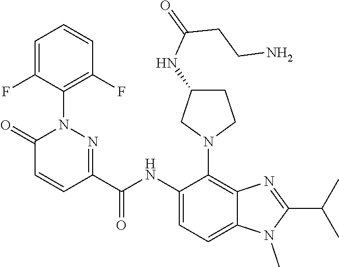 [SMILES: CC(C)c1nc2c(N3CC[C@@H](NC(=O)CCN)C3)c(NC(=O)c3ccc(=O)n(-c4c(F)cccc4F)n3)ccc2n1C] | HCOOH salt | LC-MS m/z: 579.3 (M + 1)⁺ ¹H NMR (400 MHz, CD₃OD) δ 8.55 (s, 1H), 8.21 (dd, J = 9.3, 2.2 Hz, 2H), 7.67 (tt, J = 8.5, 6.2 Hz, 1H), 7.39-7.21 (m, 4H), 4.37 (h, J = 4.6, 4.1 Hz, 1H), 3.79 (s, 3H), 3.60 (h, J = 5.9 Hz, 2H), 3.49 (q, J = 8.4 Hz, 1H), 3.40-3.34 (m, 1H), 3.24 (td, J = 8.2, 3.4 Hz, 1H), 3.20-3.12 (m, 2H), 2.60 (td, J = 6.4, 2.4 Hz, 2H), 2.00- 1.80 (m, 2H), 1.49-1.37 (m, 6H). |
| | (R)-N-(4-(3-(3- | | |
| | aminopropanamido)pyrrolidin-1- | | |
| | yl)-2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 129 | 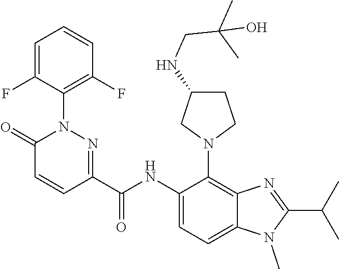 [SMILES: CC(C)c1nc2c(N3CC[C@@H](NCC(C)(C)O)C3)c(NC(=O)c3ccc(=O)n(-c4c(F)cccc4F)n3)ccc2n1C] | Free base | LC-MS m/z: 580.3 (M + 1)⁺ ¹H NMR (400 MHz, CD₃OD) δ 8.21 (dd, J = 9.3, 5.2 Hz, 2H), 7.69 (tt, J = 8.5, 6.2 Hz, 1H), 7.39- 7.19 (m, 4H), 3.77 (s, 3H), 3.72- 3.57 (m, 2H), 3.50 (dd, J = 9.8 6.1 Hz, 1H), 3.41-3.32 (m, 2H), 3.21 (ddd, J = 9.1, 7.7, 5.0 Hz, 1H), 2.69-2.55 (m, 2H), 2.08- 1.95 (m, 1H), 1.82 (dt, J = 12.5, 5.7 Hz, 1H), 1.41 (d, J = 6.9 Hz, 6H), 1.25 (d, J = 2.9 Hz, 6H). |
| | (R)-1-(2,6-difluorophenyl)-N-(4-(3- | | |
| | ((2-hydroxy-2- | | |
| | methylpropyl)amino)pyrrolidin-1- | | |
| | yl)-2-isopropyl-1-methyl-1H- | | |
| | benzo[d]imidazol-5-yl)-6-oxo-1,6- | | |
| | dihydropyridazine-3-carboxamide | | |
| Example 130 | 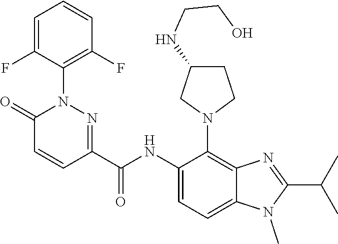 [SMILES: CC(C)c1nc2c(N3CC[C@@H](NCCO)C3)c(NC(=O)c3ccc(=O)n(-c4c(F)cccc4F)n3)ccc2n1C] | Freebase | LC-MS m/z: 552.3 (M + 1)⁺ ¹H NMR (400 MHz, CD₃OD) δ 8.19 (dd, J = 12.0, 9.4 Hz, 2H), 7.76-7.63 (m, 2H), 7.40-7.28 (m, 3H), 4.05 (s, 3H), 3.86 (dt, J = 12.6, 5.4 Hz, 4H), 3.79-3.68 (m, 2H), 3.68-3.61 (m, 1H), 3.27 (dt, J = 8.6, 4.2 Hz, 1H), 3.24- 3.08 (m, 2H), 2.33-2.14 (m, 2H), 1.64 (dd, J = 10.4, 7.0 Hz, 6H). |
| | (R)-1-(2,6-difluorophenyl)-N-(4-(3- | | |
| | ((2- | | |
| | hydroxyethyl)amino)pyrrolidin-1- | | |
| | yl)-2-isopropyl-1-methyl-1H- | | |
| | benzo[d]imidazol-5-yl)-6-oxo-1,6- | | |
| | dihydropyridazine-3-carboxamide | | |
| Example 131 | 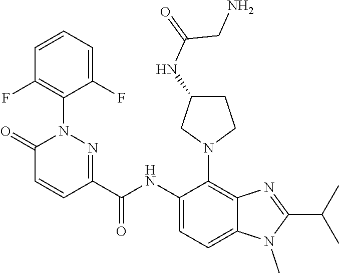 [SMILES: CC(C)c1nc2c(N3CC[C@@H](NC(=O)CN)C3)c(NC(=O)c3ccc(=O)n(-c4c(F)cccc4F)n3)ccc2n1C] | HCOOH salt | LC-MS m/z: 565.3 (M + 1)⁺ ¹H NMR (400 MHz, CD₃OD) δ 8.21 (dd, J = 9.3, 7.0 Hz, 2H), 7.67 (tt, J = 8.5, 6.2 Hz, 1H), 7.30 (ddd, J = 24.7, 17.2, 9.2 Hz, 4H), 4.43-4.33 (m, 1H), 3.78 (s, 3H), 3.60 (qd, J = 10.2, 5.4 Hz, 2H), 3.54-3.43 (m, 3H), 3.41-3.33 (m, 1H), 3.23 (td, J = 8.3, 3.6 Hz, 1H), 2.81 (s, 1H), 1.95 (dt, J = 15.3, 7.6 Hz, 1H), 1.85 (ddt, J = 12.0, 7.6, 3.9 Hz, 1H), 1.43 (d, J = 6.9 Hz, 6H). |
| | (R)-N-(4-(3-(2- | | |
| | aminoacetamido)pyrrolidin-1-yl)- | | |
| | 2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 132 | 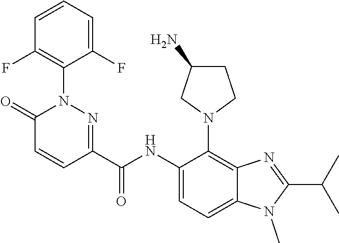 [SMILES: CC(C)c1nc2c(N3CC[C@H](N)C3)c(NC(=O)c3ccc(=O)n(-c4c(F)cccc4F)n3)ccc2n1C] | HCOOH salt | LC-MS m/z: 508.3 (M + 1)⁺ ¹H NMR (400 MHz, CD₃OD) δ 8.10 (d, J = 9.8 Hz, 1H), 8.04 (m, 1H), 7.64-7.52 (m, 2H), 7.24- 7.20 (m, 3H), 3.93 (s, 3H), 3.73 (s, 2H), 3.65-3.61 (m, 2H), 3.38 (m, 1H), 3.19-3.13 (m, 1H), 2.15-2.09 (m, 2H), 1.53-1.49 (m, 6H). |
| | (S)-N-(4-(3-aminopyrrolidin-1-yl)- | | |
| | 2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 133 | 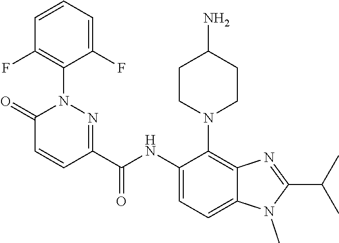 [SMILES: CC(C)c1nc2c(N3CCC(N)CC3)c(NC(=O)c3ccc(=O)n(-c4c(F)cccc4F)n3)ccc2n1C] | HCl salt | LC-MS m/z: 522.7 (M + 1)+ 1H NMR (400 MHz, CD₃OD) δ 8.57 (s, 1H), 8.14 (d, J = 9.9 Hz, 1H), 7.66-7.59 (m, 2H), 7.37- 7.15 (m, 3H), 3.94 (s, 3H), 3.78- 3.65 (m, 3H), 3.11-3.09 (m, 1H), 2.87-2.81 (s, 2H), 1.70 (s, 3H), 1.54 (d, J = 5.5 Hz, 6H). |
| | N-(4-(4-aminopiperidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 134 | 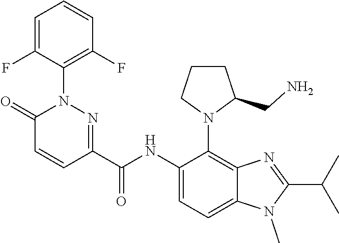 [SMILES: CC(C)c1nc2c(N3CCC[C@H]3CN)c(NC(=O)c3ccc(=O)n(-c4c(F)cccc4F)n3)ccc2n1C] | HCl salt | LC-MS m/z: 522.3 (M + 1)+ 1H NMR (400 MHz, CD₃OD) δ 8.57 (s, 1H), 8.24 (d, J = 9.9 Hz, 1H), 7.78 (m, 1H), 7.60 (m, 1H), 7.35-7.20 (m, 3H), 4.05 (s, 3H), 3.98 (m, 1H), 3.75 (m, 1H), 3.42 (m, 1H), 3.33 (m, 1H), 2.68 (m, 2H), 2.38 (m, 1H), 1.95 (m, 1H), 1.75 (m, 1H), 1.55 (m, 1H), 1.52 (d, J = 5.5 Hz, 6H). |
| | (S)-N-(4-(2- | | |
| | (aminomethyl)pyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 135 | 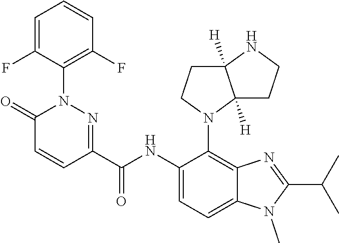 [SMILES: CC(C)c1nc2c(N3CC[C@H]4NCC[C@H]43)c(NC(=O)c3ccc(=O)n(-c4c(F)cccc4F)n3)ccc2n1C] | HCOOH salt | LC-MS m/z: 532.3 (M + 1)+ 1H NMR (400 MHz, CD₃OD) δ 8.27-8.17 (m, 2H), 7.79-7.67 (m, 2H), 7.41-7.30 (m, 3H), 4.59-4.47 (m, 2H), 4.06 (s, 3H), 3.78-3.63 (m, 2H), 3.49 (d, J = 8.5 Hz, 1H), 3.29-3.17 (m, 2H), 2.55-2.44 (m, 1H), 2.19-2.07 (m, 1H), 1.89-1.78 (m, 2H), 1.63 (dd, J = 7.0, 13.8 Hz, 6H). |
| | 1-(2,6-difluorophenyl)-N-(4- | | |
| | ((3aR,6aR)-hexahydropyrrolo[3,2- | | |
| | b]pyrrol-1(2H)-yl)-2-isopropyl-1- | | |
| | methyl-1H-benzo[d]imidazol-5- | | |
| | yl)-6-oxo-1,6-dihydropyridazine- | | |
| | 3-carboxamide | | |
| Example 136 | 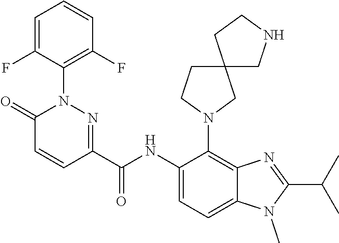 [SMILES: CC(C)c1nc2c(N3CCC4(CCNC4)C3)c(NC(=O)c3ccc(=O)n(-c4c(F)cccc4F)n3)ccc2n1C] | HCl salt | LC-MS m/z: 548.3 (M + 1)+ 1H NMR (400 MHz, CD₃OD) δ 8.48 (br d, J = 8.5 Hz, 1H), 8.27 (d, J = 9.9 Hz, 1H), 7.76-7.68 (m, 2H), 7.40-7.32 (m, 3H), 4.05 (s, 3H), 3.84-3.67 (m, 1H), 3.55- 3.39 (m, 5H), 3.25-3.10 (m, 3H), 2.27-2.16 (m, 1H), 2.14- 1.99 (m, 2H), 1.59 (d, J = 6.9 Hz, 7H). |
| | 1-(2,6-difluorophenyl)-N-(2- | | |
| | isopropyl-1-methyl-4-(2,7- | | |
| | diazaspiro[4.4]nonan-2-yl)-1H- | | |
| | benzo[d]imidazol-5-yl)-6-oxo-1,6- | | |
| | dihydropyridazine-3-carboxamide | | |
| Example 137 | 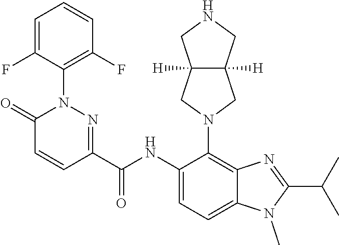 [SMILES: CC(C)c1nc2c(N3C[C@H]4CNC[C@H]4C3)c(NC(=O)c3ccc(=O)n(-c4c(F)cccc4F)n3)ccc2n1C] | HCl salt | LC-MS m/z: 534.3 (M + 1)+ 1H NMR (400 MHz, CD₃OD) δ 8.65 (br d, J = 8.82 Hz, 1 H), 8.25 (d, J = 9.90 Hz, 1 H), 7.71-7.82 (m, 2 H), 7.31-7.44 (m, 3 H), 4.08 (s, 3 H), 3.80 (dt, J = 14.13, 7.12 Hz, 1 H), 3.57-3.65 (m, 2 H), 3.41-3.52 (m, 5 H), 3.20 (br dd, J = 12.04, 7.03 Hz, 2 H), 2.86 (br s, 2 H), 1.65-1.68 (m, 6 H). |
| | 1-(2,6-difluorophenyl)-N-(4- | | |
| | ((3aR,6aS)-hexahydropyrrolo[3,4- | | |
| | c]pyrrol-2(1H)-yl)-2-isopropyl-1- | | |
| | methyl-1H-benzo[d]imidazol-5- | | |
| | yl)-6-oxo-1,6-dihydropyridazine- | | |
| | 3-carboxamide | | |
| Example 138 (isomer 1) | 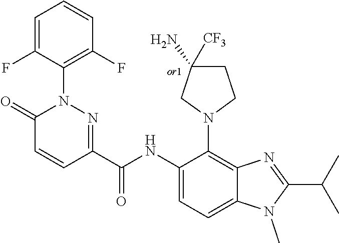 [SMILES: CC(C)c1nc2c(N3CC[C@](N)(C(F)(F)F)C3)c(NC(=O)c3ccc(=O)n(-c4c(F)cccc4F)n3)ccc2n1C] | Free Base | LC-MS m/z: 576.3 (M + 1)+ 1H NMR (400 MHz, CD₃OD) δ 8.47-8.42 (m, 1H), 8.24-8.19 (m, 1H), 7.72-7.61 (m, 1H), 7.55-7.48 (m, 1H), 7.33-7.24 (m, 3H), 3.92-3.88 (m, 3H), 3.75-3.68 (m, 1H), 3.60-3.52 (m, 2H), 3.51-3.39 (m, 2H), 2.40-2.20 (m, 2H), 1.50-1.43 (m, 6H). |
| | rel-(R)-N-(4-(3-amino-3- | | |
| | (trifluoromethyl)pyrrolidin-1-yl)- | | |
| | 2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 139 (isomer 2) | 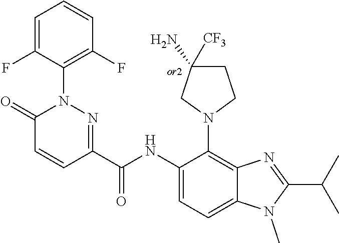 [SMILES: CC(C)c1nc2c(N3CC[C@](N)(C(F)(F)F)C3)c(NC(=O)c3ccc(=O)n(-c4c(F)cccc4F)n3)ccc2n1C] | Free Base | LC-MS m/z: 576.3 (M + 1)+ 1H NMR (400 MHz, CD₃OD) δ 8.47-8.41 (m, 1H), 8.25-8.19 (m, 1H), 7.72-7.62 (m, 1H), 7.55-7.47 (m, 1H), 7.34-7.23 (m, 3H), 3.91-3.87 (m, 3H), 3.74-3.67 (m, 1H), 3.59-3.53 (m, 2H), 3.49-3.41 (m, 2H), 2.51-2.14 (m, 2H), 1.51-1.44 (m, 6H). |
| | rel-(R)-N-(4-(3-amino-3- | | |
| | (trifluoromethyl)pyrrolidin-1-yl)- | | |
| | 2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 140 | 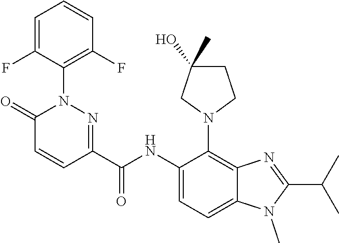 [SMILES: CC(C)c1nc2c(N3CC[C@@](C)(O)C3)c(NC(=O)c3ccc(=O)n(-c4c(F)cccc4F)n3)ccc2n1C] | HCOOH Salt | LC-MS m/z: 523.3 (M + 1)+ 1H NMR (400 MHz, CD₃OD) δ 8.62 (br s, 1H), 8.24 (d, J = 9.8 Hz, 1H), 7.83-7.60 (m, 2H), 7.34 (br d, J = 9.3 Hz, 3H), 4.02 (br s, 3H), 3.62 (br, s, 1H), 3.47-3.35 (m, 4H), 2.07 (br d, J = 11.9 Hz, 1H), 1.83 (br d, J = 8.6 Hz, 1H), 1.50 (br d, J = 6.8 Hz, 7H). |
| | (R)-1-(2,6-difluorophenyl)-N-(4- | | |
| | (3-hydroxy-3-methylpyrrolidin-1- | | |
| | yl)-2-isopropyl-1-methyl-1H- | | |
| | benzo[d]imidazol-5-yl)-6-oxo-1,6- | | |
| | dihydropyridazine-3-carboxamide | | |
| Example 141 | 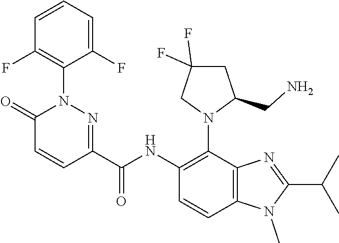 [SMILES: CC(C)c1nc2c(N3CC(F)(F)C[C@H]3CN)c(NC(=O)c3ccc(=O)n(-c4c(F)cccc4F)n3)ccc2n1C] | HCOOH Salt | LC-MS m/z: 558.3 (M + 1)+ 1H NMR (400 MHz, CD₃OD) δ 8.24 (d, J = 8.8 Hz, 1H), 8.11 (d, J = 9.9 Hz, 1H), 7.57 (tt, J = 8.6, 6.2 Hz, 1H), 7.27-7.13 (m, 4H), 4.35 (dt, J = 11.5, 5.8 Hz, 1H), 3.86 (ddd, J = 18.2, 13.5, 10.4 Hz, 1H), 3.67 (s, 3H), 3.33 (td, J = 12.2, 4.8 Hz, 1H), 2.49 (dtd, J = 16.3, 13.5, 7.9 Hz, 1H), 2.36 (qd, J = 13.1, 5.4 Hz, 2H), 1.92- 1.73 (m, 1H), 1.30 (dd, J = 6.9, 1.3 Hz. 6H). |
| | (S)-N-(4-(2-(aminomethyl)-4,4- | | |
| | difluoropyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 142 | 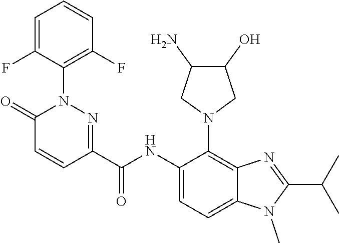 [SMILES: CC(C)c1nc2c(N3CC(N)C(O)C3)c(NC(=O)c3ccc(=O)n(-c4c(F)cccc4F)n3)ccc2n1C] | HCl Salt | LC-MS m/z: 524.3 (M + 1)+ 1H NMR (400 MHz, CD₃OD) δ 8.22-8.18 (m, 1H), 8.18-8.09 (m, 1H), 7.67-7.58 (m, 2H), 7.31-7.23 (m, 3H), 4.55-4.51 (m, 1H), 4.07-3.83 (m, 5H), 3.68-3.57 (m, 2H), 3.45-3.36 (m, 1H), 3.26-3.21 (m, 1H), 1.63-1.47 (m, 6H). |
| | N-(4-(3-amino-4- | | |
| | hydroxypyrrolidin-1-yl)-2- | | |
| | isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |

### Example 143: (R)—N-(4-(3-aminopyrrolidin-1-yl)-7-fluoro-2-isopropyl-1-methyl-1H-benzo[d]imidazol-5-yl)-1-(2,6-difluorophenyl)-6-oxo-1,6-dihydropyridazine-3-carboxamide

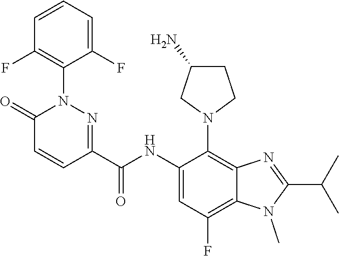 [SMILES: CC(C)c1nc2c(N3CC[C@@H](N)C3)c(NC(=O)c3ccc(=O)n(-c4c(F)cccc4F)n3)cc(F)c2n1C]

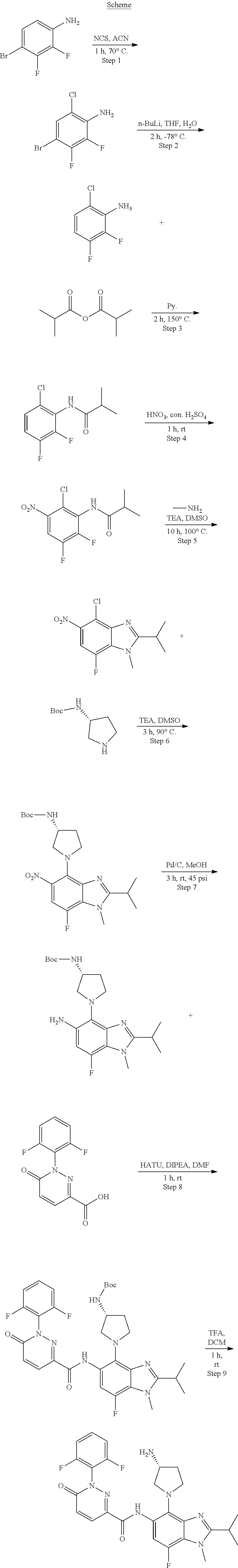

### Step 1: 4-bromo-6-chloro-2,3-difluoroaniline

To a solution of 4-bromo-2,3-difluoroaniline (10 g, 48.08 mmol, 1 eq.) in ACN (80 mL) was added NCS (7.06 g, 52.88 mmol, 1.1 eq.) at 70° C. in portions. The reaction was stirred for 1 hour at 70° C. to give a black brown solution. LCMS showed the reaction the starting material was consumed up and one new major peak was detected. The reaction was diluted with water and extracted with EA (80 mL×3). The combined organic layers were washed with brine, dried over Na₂SO₄, filtered and concentrated to afford 4-bromo-6-chloro-2,3-difluoroaniline (12.21 g, Yield: 104.7%) as dark oil.

LC-MS m/z: 242.1 (M+1)⁺

### Step 2: 6-chloro-2,3-difluoroaniline

To a solution of 4-bromo-6-chloro-2,3-difluoroaniline (48.08 mmol, 1 eq.) in THF (100 mL) was added n-BuLi (54.09 mL, 86.54 mmol, 1.8 eq.) dropwise at −78° C. The reaction was stirred for 2 hours at −78° C. to give a black brown solution. The reaction was quenched with H₂O and LCMS showed the reaction the starting material was consumed up and one new major peak was detected. The reaction was diluted with H₂O and extracted with EA (100 mL×3). The combined organic layers were washed with brine, dried over Na₂SO₄, filtered and concentrated to afford 6-chloro-2,3-difluoroaniline (7 g, Yield: 89%) as black oil.

LC-MS m/z: 164.1 (M+1)⁺

### Step 3: N-(6-chloro-2,3-difluorophenyl)isobutyramide

A solution of 6-chloro-2,3-difluoroaniline (7 g, 42.8 mmol, 1 eq.) in isobutyric anhydride (8.12 g, 51.36 mmol, 1.2 eq.) and Py. (4.06 g, 51.36 mmol, 1.2 eq.). The reaction was stirred for 2 hours at 150° C. by microwave to give a black brown solution. LCMS showed the starting material was consumed up and one major peak with desired mass was detected. The reaction was diluted with H₂O and extracted with EA (50 mL×3). The combined organic layers were washed with brine, dried over Na₂SO₄, filtered and concentrated to dryness. The residue was purified by flash column (Silica column, eluted with 5% to 10% EA in PE) to afford N-(6-chloro-2,3-difluorophenyl)isobutyramide (2.7 g, Yield: 27%) as an off-white solid.

LC-MS m/z: 234.1 (M+1)⁺

### Step 4: N-(2-chloro-5,6-difluoro-3-nitrophenyl)isobutyramide

To a solution of N-(6-chloro-2,3-difluorophenyl)isobutyramide (2.1 g, 8.99 mmol, 1 eq.) in 98% H₂SO₄ (8 ml) was added 66% HNO₃ (0.68 mL, 9.89 mmol, 1.1 eq.) dropwise at 0° C. The ice bath was removed and the reaction was stirred at rt for 1 hour to give a black brown solution. LCMS showed the starting material was consumed up and one major peak with desired mass was detected. The mixture was then poured into ice water and basified with Na₂CO₃ to pH=1^{˜}2 and then extracted with EA (30 mL×3). The combined organic layers were washed with brine, dried over Na₂SO₄, filtered and concentrated to afford N-(2-chloro-5,6-difluoro-3-nitrophenyl)isobutyramide (1.8 g, Yield:72%) as a black brown solid.

LC-MS m/z: 279.1 (M+1)⁺

### Step 5: 4-chloro-7-fluoro-2-isopropyl-1-methyl-5-nitro-1H-benzo[d]imidazole

To a solution of N-(2-chloro-5,6-difluoro-3-nitrophenyl)isobutyramide (1.8 g, 6.46 mmol, 1 eq.) and TEA (2.61 g, 25.84 mmol, 4 eq.) in DMSO (20 ml) was added methanamine hydrochloride (0.43 g, 6.46 mmol, 1 eq.). The reaction was stirred at rt for 1 hour and then warmed to 100° C. and stirred for another 10 hours to give a black brown solution. LCMS showed the starting material was consumed up and a small peak with desired mass was detected. The mixture was diluted with H₂O and extracted with EA (20 mL×3). The combined organic layers were washed with brine, dried over Na₂SO₄, filtered and concentrated to afford 4-chloro-7-fluoro-2-isopropyl-1-methyl-5-nitro-1H-benzo[d]imidazole (1.3 g, Yield: 74%) as a brown solid.

LC-MS m/z: 272.1 (M+1)⁺

### Step 6: tert-butyl (R)-(1-(7-fluoro-2-isopropyl-1-methyl-5-nitro-1H-benzo[d]imidazol-4-yl)pyrrolidin-3-yl)carbamate

To a solution of 4-chloro-7-fluoro-2-isopropyl-1-methyl-5-nitro-1H-benzo[d]imidazole (500 mg, 1.84 mmol, 1 eq.) in DMSO (5 ml) was added TEA (558.71 mg, 5.52 mmol, 3 eq.) and tert-butyl (R)-pyrrolidin-3-ylcarbamate (342.79 mg, 1.84 mmol, 1 eq.). The reaction was stirred for 3 hours at 90° C. to give a black brown solution. LCMS showed the starting material was consumed up and one major peak with desired mass was detected. The reaction was diluted with H₂O and extracted with EA (10 mL×3). The combined organic layers were washed with brine, dried over Na₂SO₄, filtered and concentrated to afford tert-butyl (R)-(1-(7-fluoro-2-isopropyl-1-methyl-5-nitro-1H-benzo[d]imidazol-4-yl)pyrrolidin-3-yl)carbamate (0.8 g, crude) as a brown solid.

LC-MS m/z: 422.3 (M+1)⁺

### Step 7: tert-butyl (R)-(1-(5-amino-7-fluoro-2-isopropyl-1-methyl-1H-benzo[d]imidazol-4-yl)pyrrolidin-3-yl)carbamate

To a solution of tert-butyl (R)-(1-(7-fluoro-2-isopropyl-1-methyl-5-nitro-1H-benzo[d]imidazol-4-yl)pyrrolidin-3-yl)carbamate (1.84 mmol, 1 eq.) in MeOH (8 mL) was added Pd/C (100 mg) under N₂. The reaction was degassed with H₂ for several times and stirred for 3 hours at rt under 45 psi to give a black brown suspension. LCMS showed the starting material was consumed up and one major peak with desired mass was detected. The reaction was filtered through a pad of Celite and the filter cake was washed with MeOH. The filtrate was concentrated to afford tert-butyl (R)-(1-(5-amino-7-fluoro-2-isopropyl-1-methyl-1H-benzo[d]imidazol-4-yl)pyrrolidin-3-yl)carbamate (0.7 g, Yield: 97.2%) as black brown solid.

LC-MS m/z: 392.3 (M+1)⁺

### Step 8: tert-butyl (R)-(1-(5-(1-(2,6-difluorophenyl)-6-oxo-1,6-dihydropyridazine-3-carboxamido)-7-fluoro-2-isopropyl-1-methyl-1H-benzo[d]imidazol-4-yl)pyrrolidin-3-yl)carbamate

To a solution of 1-(2,6-difluorophenyl)-6-oxo-1,6-dihydropyridazine-3-carboxylic acid (200 mg 60% purity, 475.86 μmol, 1 eq.) and tert-butyl (R)-(1-(5-amino-7-fluoro-2-isopropyl-1-methyl-1H-benzo[d]imidazol-4-yl)pyrrolidin-3-yl)carbamate (223.55 mg, 571.03 μmol, 1.2 eq.) in DMF (5 mL) was added HATU (271.41 mg, 713.78 μmol, 1.5 eq.) and DIEA (184.51 mg, 1.43 mmol, 3 eq.). The reaction was stirred for 1 hour at rt to give a black brown solution. LCMS showed the starting material was consumed up and one major peak with desired mass was detected. The reaction was diluted with H₂O and extracted with EA (10 mL×3). The combined organic layers were washed with brine, dried over Na₂SO₄, filtered and concentrated to afford tert-butyl (R)-(1-(5-(1-(2,6-difluorophenyl)-6-oxo-1,6-dihydropyridazine-3-carboxamido)-7-fluoro-2-isopropyl-1-methyl-1H-benzo[d]imidazol-4-yl)pyrrolidin-3-yl)carbamate (450 mg, crude) as brown oil.

LC-MS m/z: 626.3 (M+1)⁺

### Step 9: (R)—N-(4-(3-aminopyrrolidin-1-yl)-7-fluoro-2-isopropyl-1-methyl-1H-benzo[d]imidazol-5-yl)-1-(2,6-difluorophenyl)-6-oxo-1,6-dihydropyridazine-3-carboxamide

To a solution of tert-butyl (R)-(1-(5-(1-(2,6-difluorophenyl)-6-oxo-1,6-dihydropyridazine-3-carboxamido)-7-fluoro-2-isopropyl-1-methyl-1H-benzo[d]imidazol-4-yl)pyrrolidin-3-yl)carbamate (475.86 μmol, 1 eq.) in DCM (6 mL) was added TFA (3 mL). The reaction was stirred for 1 hour at rt to give a brown solution. LCMS showed the starting material was consumed up and one major peak with desired mass was detected. The reaction was basified with con. Na₂CO₃ aq. to pH=10^{˜}11 and extracted with DCM (10 mL×3). The combined organic layers were washed with brine, dried over Na₂SO₄, filtered and concentrated to dryness. The residue was purified by flash column (C₁₈ column, eluting a gradient of ACN/H₂O containing 0.1% HCOOH, at flow rate of 30 mL/min). The collected flows were basified with con. Na₂CO₃ aq. to pH=10^{˜}11 and extracted with EA (30 mL×3). The combined organic layers were washed with brine, dried over Na₂SO₄, filtered and concentrated to dryness. The residue was purified by flash column (Silica column, eluted with 10% MeOH in DCMO. The collected flows were concentrated and lyophilized with 2 M HCl. aq. (3 drops) to afford Example 143 (43 mg, 17.2%) of as a brown solid.

LC-MS m/z: 526.3 (M+1)⁺

¹H NMR (400 MHz, CD₃OD) δ 8.27-8.17 (m, 2H), 7.69 (tt, J=8.6, 6.2 Hz, 1H), 7.38-7.29 (m, 3H), 4.01 (d, J=0.9 Hz, 3H), 4.01-3.96 (m, 1H), 3.64 (dd, J=12.6, 4.9 Hz, 1H), 3.49-3.33 (m, 3H), 2.35-2.24 (m, 1H), 2.20-2.07 (m, 1H), 1.42 (dd, J=6.8, 3.2 Hz, 6H).

| No. | Structure | Salt form | MS and ¹HNMR |
|---|---|---|---|
| Example 144 | 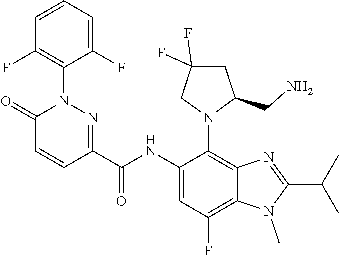 [SMILES: CC(C)c1nc2c(N3CC(F)(F)C[C@H]3CN)c(NC(=O)c3ccc(=O)n(-c4c(F)cccc4F)n3)cc(F)c2n1C] | HCOOH salt | LC-MS m/z: 576.2 (M + 1)⁺ ¹H NMR (400 MHz, CD₃OD) δ 8.22 (d, J = 9.9 Hz, 1H), 7.84 (dd, J = 13.2, 5.6 Hz, 1H), 7.73-7.61 (m, 1H), 7.35-7.24 (m, 3H), 4.75 (d, J = 7.3 Hz, 1H), 3.97 (d, J = 1.7 Hz, 3H), 3.88-3.71 (m, 1H), 3.72-3.54 (m, 2H), 2.94- 2.69 (m, 3H), 2.24-2.07 (m, 1H), 1.41 (dt, J = 5.6, 1.5 Hz, 6H). |
| | (S)-N-(4-(2-(aminomethyl)-4,4- | | |
| | difluoropyrrolidin-1-yl)-7-fluoro- | | |
| | 2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |
| Example 145 | 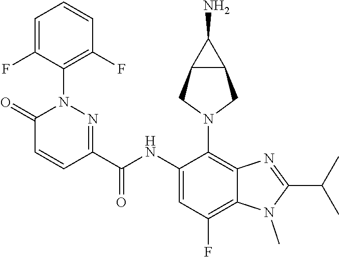 [SMILES: CC(C)c1nc2c(N3C[C@@H]4[C@H](N)[C@@H]4C3)c(NC(=O)c3ccc(=O)n(-c4c(F)cccc4F)n3)cc(F)c2n1C] | HCOOH salt | LC-MS m/z: 538.2 (M + 1)⁺ ¹H NMR (400 MHz, CD₃OD): δ 8.20 (d, J = 9.8 Hz, 1H), 7.75- 7.60 (m, 2H), 7.31 (dd, J = 9.1, 7.3 Hz, 3H), 4.03 (s, 3H), 3.88- 3.77 (m, 2H), 3.54-3.38 (m, 3H), 2.95 (t, J = 7.2 Hz, 1H), 2.06- 1.95 (m, 2H), 1.46 (d, J = 6.9 Hz, 6H). |
| | N-(4-((1R,5S,6r)-6-amino-3- | | |
| | azabicyclo[3.1.0]hexane-3-yl)-7- | | |
| | fluoro-2-isopropyl-1-methyl-1H- | | |
| | benzo[d]imidazol-5-yl)-1-(2,6- | | |
| | difluorophenyl)-6-oxo-1,6- | | |
| | dihydropyridazine-3-carboxamide | | |

### Example 146: (R)—N-(4-(3-aminopyrrolidin-1-yl)-1-methyl-2-(1-(trifluoromethyl)cyclopropyl)-1H-benzo[d]imidazol-5-yl)-1-(2,6-difluorophenyl)-6-oxo-1,6-dihydropyridazine-3-carboxamide

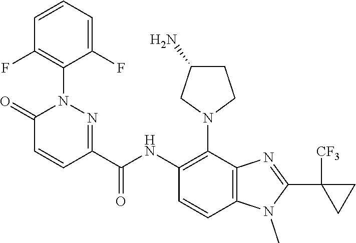 [SMILES: Cn1c(C2(C(F)(F)F)CC2)nc2c(N3CC[C@@H](N)C3)c(NC(=O)c3ccc(=O)n(-c4c(F)cccc4F)n3)ccc21]

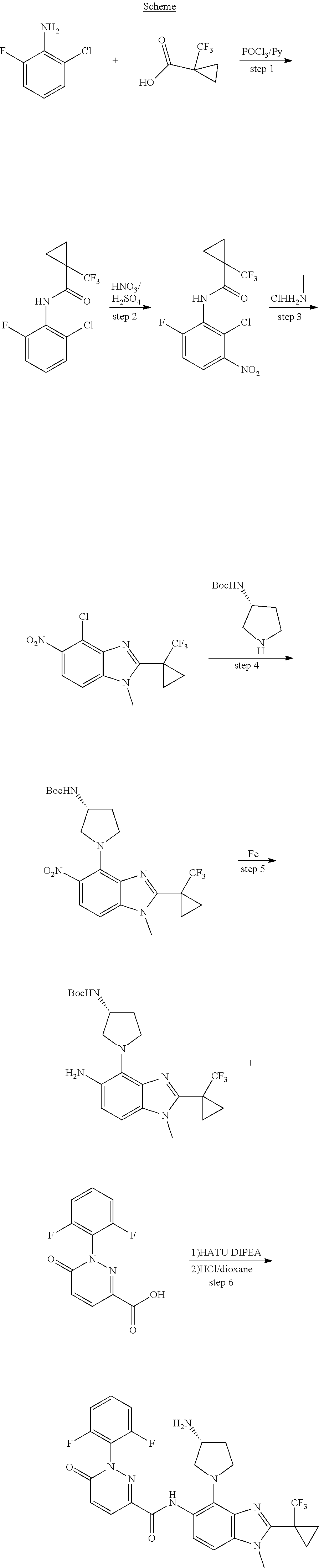

### Step1: N-(2-chloro-6-fluorophenyl)-1-(trifluoromethyl)cyclopropane-1-carboxamide

To a mixture of 2-chloro-6-fluoroaniline (1.46 g, 10.03 mmol, 1.0 eq), 1-(trifluoromethyl)cyclopropane-1-carboxylic acid (1.54 g, 10.03 mmol, 1.00 eq) in pyridine 10 Ml, was added POCl₃ (2.50 g 16.33 mmol 1.60 eq). The mixture was stirred at 25° C. for 1 h as a black solution. LCMS analysis of the reaction mixture showed the reaction was completed. The mixture was poured into water 100 mL, then was filtered, the filtrate cake was dryness in vacuo to afford N-(2-chloro-6-fluorophenyl)-1-(trifluoromethyl)cyclopropane-1-carboxamide (2.20 g) as a white solid.

LC-MS m/z: 282.6 (M+1)⁺.

### Step2: N-(2-chloro-6-fluoro-3-nitrophenyl)-1-(trifluoromethyl)cyclopropane-1-carboxamide

To a solution of N-(2-chloro-6-fluorophenyl)-1-(trifluoromethyl)cyclopropane-1-carboxamide (2.20 g 7.82 mol 1.00 eq) in concentrated sulfuric acid 7 mL, was added fuming nitric acid 0.6 mL at 0° C., and was stirred at 0° C. for 1 h as a brown solution. LCMS analysis of the reaction mixture showed full conversion to the desired product. The residue was poured into water 100 mL, then was filtered, the filtrate cake was dryness in vacuo to afford N-(2-chloro-6-fluoro-3-nitrophenyl)-1-(trifluoromethyl)cyclopropane-1-carboxamide (2.30 g) as a light yellow solid.

LC-MS m/z: 327.6 (M+1)⁺.

### Step 3: 4-chloro-1-methyl-5-nitro-2-(1-(trifluoromethyl)cyclopropyl)-1H-benzo[d]imidazole

A mixture of N-(2-chloro-6-fluoro-3-nitrophenyl)-1-(trifluoromethyl)cyclopropane-1-carboxamide (2.30 g 7.18 mmol 1.0 eq), CH₃NH₂HCl (0.53 g 7.90 mmol 1.10 eq), Et₃N 2 mL and DMSO 20 mL, was stirred at 120° C. for 3 h as a brown suspension. Target compound MS was detected by LC-MS. The mixture was poured into water 100 mL, extracted with DCM (30 mL×2). The mixture of reaction was concentrated to dryness in vacuo. The resulting residue was purified by column (SiO₂, eluted with 80%-90% EA inPE, COMBI FLASH NEXTAGEN 300) to afford 4-chloro-1-methyl-5-nitro-2-(1-(trifluoromethyl)cyclopropyl)-1H-benzo[d]imidazole (1.2 g) as a yellow solid.

LC-MS m/z: 320.7 (M+1)⁺.

### Step 4: tert-butyl (R)-(1-(1-methyl-5-nitro-2-(1-(trifluoromethyl)cyclopropyl)-1H-benzo[d]imidazol-4-yl)pyrrolidin-3-yl)carbamate

A mixture of 4-chloro-1-methyl-5-nitro-2-(1-(trifluoromethyl)cyclopropyl)-1H-benzo[d]imidazole (2.20 g, 6.88 mmol, 1.0 eq), tert-butyl (R)-pyrrolidin-3-ylcarbamate (1.67 g, 8.95 mmol, 1.30 eq) and dioxane 20 mL and Et₃N 2 Ml. The reaction mixture was stirred at 100° C. for 4 h as a brown suspension. Target compound MS was detected by LC-MS. The mixture of reaction was concentrated to dryness in vacuo. The resulting residue was purified by column (SiO₂, eluted with 50%-60% EA inPE, COMBI FLASH NEXTAGEN 300) to afford tert-butyl (R)-(1-(1-methyl-5-nitro-2-(1-(trifluoromethyl)cyclopropyl)-1H-benzo[d]imidazol-4-yl)pyrrolidin-3-yl)carbamate (1.4 g) as a yellow solid.

LC-MS m/z: 470.5 (M+1)⁺.

### Step 5: tert-butyl (R)-(1-(5-amino-1-methyl-2-(1-(trifluoromethyl)cyclopropyl)-1H-benzo[d]imidazol-4-yl)pyrrolidin-3-yl)carbamate

To a solution of tert-butyl (R)-(1-(1-methyl-5-nitro-2-(1-(trifluoromethyl)cyclopropyl)-1H-benzo[d]imidazol-4-yl)pyrrolidin-3-yl)carbamate (0.40 g 0.85 mmol 1.00 eq) in EtOH 10 mL, was added Fe (0.24 g 4.25 mmol 5.00 eq), AcOH (1 mL), was stride for 5 h at 25° C. as a black suspension, Target compound MS was detected by LC-MS and no SM remain. The mixture of reaction was filtered, the filtrate was concentrated, was added NaHCO₃ solution to PH=8, was exacted by EA 30 mL, then to dryness in vacuo affording tert-butyl (R)-(1-(5-amino-1-methyl-2-(1-(trifluoromethyl)cyclopropyl)-1H-benzo[d]imidazol-4-yl)pyrrolidin-3-yl)carbamate (0.40 g) as a red solid which was directly used to the next reaction.

LC-MS m/z: 440.5 (M+1)⁺.

### Step 6: (R)—N-(4-(3-aminopyrrolidin-1-yl)-1-methyl-2-(1-(trifluoromethyl)cyclopropyl)-1H-benzo [d]imidazol-5-yl)-1-(2,6-difluorophenyl)-6-oxo-1,6-dihydropyridazine-3-carboxamide

To a solution of 1-(2,6-difluorophenyl)-6-oxo-1,6-dihydropyridazine-3-carboxylic acid (150 mg, 0.43 mmol, 1.00 eq.), tert-butyl (R)-(1-(5-amino-1-methyl-2-(1-(trifluoromethyl)cyclopropyl)-1H-benzo[d]imidazol-4-yl)pyrrolidin-3-yl)carbamate (251 mg, 0.57 mmol, 1.34 eq.) in ACN (10 mL) was added DIEA0.3 mL, follow by HATU (245 mg, 0.65 mmol, 1.5 eq.) and stirred for 3 h to get a black suspension. LC-MS showed most of the start material was consumed and one peak was detected. The reaction was diluted with H₂O and extracted with EA. The combined organic layers were dried over Na₂SO₄, filtered and concentrated. The residue was purified by FLASH column (EA in PE 50%-60%, COMBI FLASH NEXTAGEN 300) to afford intermediate 170 mg as yellow solid. To intermediate was added 10 ML dioxane and 10 mL HCl/dioxane (4 mol/L), and stirred 4 h as a yellow suspension. The reaction mixture was concentrated to afford Example 146 (78 mg) as a yellow solid.

LC-MS m/z: 574.5 (M+1)⁺.

¹H NMR (400 MHz, CD₃OD) δ 8.35 (d, J=8.9 Hz, 1H), 8.24 (d, J=9.8 Hz, 1H), 7.70 (td, J=8.7, 4.2 Hz, 1H), 7.50 (d, J=9.0 Hz, 1H), 7.34 (dd, J=9.2, 6.1 Hz, 3H), 4.08-3.89 (m, 4H), 3.71 (dd, J=12.0, 5.9 Hz, 1H), 3.65-3.48 (m, 3H), 2.28-2.13 (m, 2H), 1.71 (s, 2H), 1.52 (s, 2H).

| No. | Structure | Salt form | MS and ¹HNMR |
|---|---|---|---|
| Example 147 | 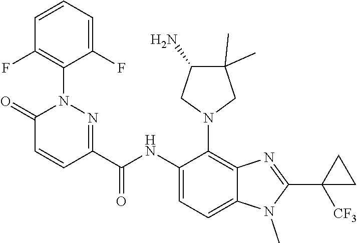 [SMILES: Cn1c(C2(C(F)(F)F)CC2)nc2c(N3C[C@H](N)C(C)(C)C3)c(NC(=O)c3ccc(=O)n(-c4c(F)cccc4F)n3)ccc21] | HCOOH Salt | LC-MS m/z: 602.6 (M + 1)⁺. ¹H NMR (400 MHz, CD₃OD) δ 8.54 (d, J = 8.9 Hz, 1H), 8.25 (d, J = 9.8 Hz, 1H), 7.68 (tt, J = 8.6, 6.2 Hz, 1H), 7.50 (d, J = 9.0 Hz, 1H), 7.35- 7.20 (m, 3H), 4.05-3.94 (m, 3H), 3.93 (dt, J = 11.4, 3.8 Hz, 1H), 3.47 (td, J = 7.4, 1.5 Hz, 2H), 3.38-3.34 (m, 1H), 3.01 (d, J = 9.3 Hz, 1H), 1.77-1.61 (m, 2H), 1.48 (t, J = 24.5 Hz, 2H), 1.21 (s, 3H), 0.97 (s, 3H). |
| | (R)-N-(4- | | |
| | (4-amino-3,3- | | |
| | dimethylpyrrolidin-1-yl)-1- | | |
| | methyl-2-(1- | | |
| | (trifluoromethyl)cyclopropyl)- | | |
| | 1H-benzo[d]imidazol-5-yl)-1- | | |
| | (2,6-difluorophenyl)-6-oxo-1,6- | | |
| | dihydropyridazine-3- | | |
| | carboxamide | | |
| Example 148 | 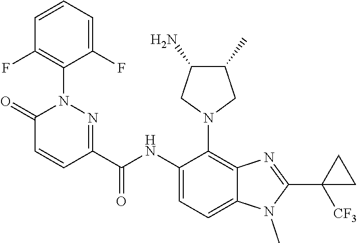 [SMILES: C[C@@H]1CN(c2c(NC(=O)c3ccc(=O)n(-c4c(F)cccc4F)n3)ccc3c2nc(C2(C(F)(F)F)CC2)n3C)C[C@@H]1N] | HCOOH Salt | LC-MS m/z: 588.3 (M + 1)⁺. ¹H NMR (400 MHz, CD₃OD) δ 8.56 (s, 1H), 8.35 (d, J = 8.9 Hz, 1H), 8.21 (d, J = 9.8 Hz, 1H), 7.80-7.62 (m, 1H), 7.43 (d, J = 8.9 Hz, 1H), 7.32 (dd, J = 17.3, 9.1 Hz, 3H), 3.95 (s, 3H), 3.81 (dd, J = 11.5, 5.5 Hz, 1H), 3.70 (d, J = 5.0 Hz, 1H), 3.39 (t, J = 8.8 Hz, 2H), 3.27 (d, J = 9.5 Hz, 3H), 2.50-2.34 (m, 1H), 1.66 (d, J = 21.7 Hz, 2H), 1.53 (d, J = 21.5 Hz, 2H), 1.15 (d, J = 7.0 Hz, 3H). |
| | N-(4- | | |
| | ((3R,4R)-3-amino-4- | | |
| | methylpyrrolidin-1-yl)-1-methyl- | | |
| | 2-(1- | | |
| | (trifluoromethyl)cyclopropyl)- | | |
| | 1H-benzo[d]imidazol-5-yl)-1- | | |
| | (2,6-difluorophenyl)-6-oxo-1,6- | | |
| | dihydropyridazine-3- | | |
| | carboxamide | | |
| Example 149 | 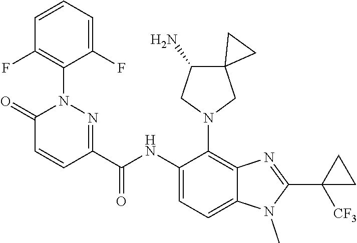 [SMILES: Cn1c(C2(C(F)(F)F)CC2)nc2c(N3C[C@H](N)C4(CC4)C3)c(NC(=O)c3ccc(=O)n(-c4c(F)cccc4F)n3)ccc21] | HCl Salt | LC-MS m/z: 600.3 (M + 1)⁺. ¹H NMR (400 MHz, CD₃OD) δ 8.50 (d, J = 8.9 Hz, 1H), 8.26 (d, J = 9.9 Hz, 1H), 7.80-7.62 (m, 1H), 7.53 (d, J = 9.0 Hz, 1H), 7.35 (dd, J = 14.0, 7.1 Hz, 3H), 4.00 (d, J = 9.3 Hz, 3H), 3.95 (dd, J = 12.2, 5.2 Hz, 1H), 3.84 (d, J = 9.5 Hz, 2H), 3.79- 3.49 (m, 6H), 2.85 (d, J = 9.6 Hz, 1H), 1.71 (d, J = 3.8 Hz, 2H), 1.53 (s, 2H), 1.09 (dt, J = 11.0, 5.6 Hz, 1H), 0.95-0.83 (m, 1H), 0.70 (d, J = 5.5 Hz, 1H), 0.38 (dd, J = 9.4, 5.2 Hz, 1H). |
| | (R)-N-(4- | | |
| | (7-amino-5-azaspiro[2.4]heptan- | | |
| | 5-yl)-1-methyl-2-(1- | | |
| | (trifluoromethyl)cyclopropyl)- | | |
| | 1H-benzo[d]imidazol-5-yl)-1- | | |
| | (2,6-difluorophenyl)-6-oxo-1,6- | | |
| | dihydropyridazine-3- | | |
| | carboxamide | | |

As former similar synthetic method:

| No. | Structure | Salt form | MS and ¹HNMR |
|---|---|---|---|
| Example 150 | 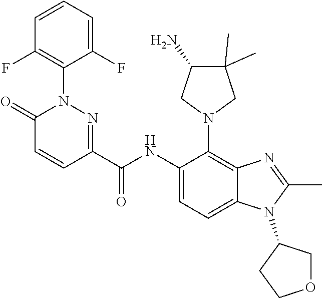 [SMILES: Cc1nc2c(N3C[C@H](N)C(C)(C)C3)c(NC(=O)c3ccc(=O)n(-c4c(F)cccc4F)n3)ccc2n1[C@H]1CCOC1] | HCOOH Salt | LC-MS m/z: 564.6 (M + 1)⁺ ¹H NMR (400 MHz, CD₃OD) δ 8.56 (s, 1H), 8.40 (d, J = 9.0 Hz, 1H), 8.28 (d, J = 9.9 Hz, 1H), 7.74-7.60 (m, 2H), 7.39-7.24 (m, 3H), 5.34 (d, J = 7.8 Hz, 1H), 4.40 (dd, J = 8.8, 5.8 Hz, 1H), 4.23 (dd, J = 10.4, 3.1 Hz, 1H), 4.04 (dd, J = 10.4, 7.8 Hz, 1H), 3.84 (dt, J = 18.6, 7.7 Hz, 2H), 3.45-3.34 (m, 2H), 2.97 (d, J = 9.2 Hz, 1H), 2.69 (s, 3H), 2.63-2.53 (m, 1H), 2.24 (dt, J = 18.6, 11.5 Hz, 1H), 1.17 (s, 3H), 0.94 (s, 3H). |
| | N-(4-((R)-4-amino-3,3- | | |
| | dimethylpyrrolidin-1-yl)-2- | | |
| | methyl-1-((S)-tetrahydrofuran-3- | | |
| | yl)-1H-benzo[d]imidazol-5-yl)-1- | | |
| | (2,6-difluorophenyl)-6-oxo-1,6- | | |
| | dihydropyridazine-3- | | |
| | carboxamide | | |
| Example 151 | 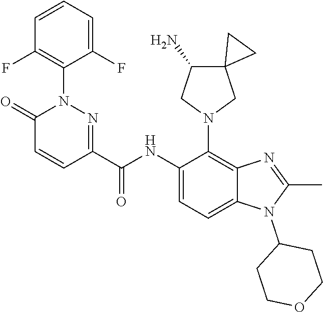 [SMILES: Cc1nc2c(N3C[C@H](N)C4(CC4)C3)c(NC(=O)c3ccc(=O)n(-c4c(F)cccc4F)n3)ccc2n1C1CCOCC1] | HCOOH | LC-MS m/z: 576.3 (M + 1)⁺ ¹H NMR (400 MHz, CD₃OD) δ 8.33 (d, J = 8.9 Hz, 1H), 8.23 (d, J = 9.8 Hz, 1H), 7.76-7.58 (m, 2H), 7.32 (td, J = 9.7, 4.2 Hz, 3H), 4.73-4.60 (m, 1H), 4.15 (dd, J = 11.7, 4.5 Hz, 2H), 3.90 (dd, J = 12.2, 5.0 Hz, 1H), 3.81 (d, J = 9.3 Hz, 1H), 3.66 (t, J = 11.8 Hz, 2H), 3.46 (d, J = 12.3 Hz, 1H), 3.27 (d, J = 4.9 Hz, 1H), 2.84-2.76 (m, 1H), 2.73 (s, 3H), 2.56 (qd, J = 12.4, 4.4 Hz, 2H), 1.88 (d, J = 11.6 Hz, 2H), 1.06 (dt, J = 10.8, 5.8 Hz, 1H), 0.81 (dt, J = 11.1, 6.1 Hz, 1H), 0.63 (p, J = 5.0 Hz, 1H), 0.34 (p, J = 5.1 Hz, 1H). |
| | 1-(2,6-difluorophenyl)-N-(4- | | |
| | [(7R)-7-amino-5- | | |
| | azaspiro[2.4]heptan-5-yl]-2- | | |
| | methyl-1-(3,4,5,6-tetrahydro- | | |
| | 2H-pyran-4-yl)benzo[d]imidazol- | | |
| | 5-yl)-6-oxo-1,2-diazine-3- | | |
| | carboxamide | | |
| Example 152 | 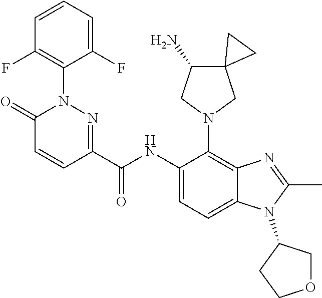 [SMILES: Cc1nc2c(N3C[C@H](N)C4(CC4)C3)c(NC(=O)c3ccc(=O)n(-c4c(F)cccc4F)n3)ccc2n1[C@H]1CCOC1] | HCOOH salt | LC-MS m/z: 562.2 (M + 1)⁺ ¹H NMR (400 MHz, CD₃OD) δ 8.22 (t, J = 9.9 Hz, 2H), 7.74-7.63 (m, 2H), 7.37-7.25 (m, 3H), 5.37 (qd, J = 8.9, 7.2, 2.8 Hz, 1H), 4.39 (td, J = 8.7, 2.8 Hz, 1H), 4.24 (dd, J = 10.5, 2.9 Hz, 1H), 4.03 (dd, J = 10.5, 7.7 Hz, 1H), 3.92 (dd, J = 11.9, 5.0 Hz, 1H), 3.79 (td, J = 9.1, 5.9 Hz, 2H), 3.45 (d, J = 11.9 Hz, 1H), 3.27 (d, J = 4.8 Hz, 1H), 2.82 (d, J = 9.2 Hz, 1H), 2.76 (s, 3H), 2.61 (dddd, J = 12.9, 9.6, 6.9, 2.8 Hz, 1H), 2.29-2.17 (m, 1H), 1.05 (dt, J = 9.9, 5.8 Hz, 1H), 0.79 (dt, J = 9.5, 5.9 Hz, 1H), 0.62 (dt, J = 10.4, 5.3 Hz, 1H), 0.38 (ddd, J = 10.0, 6.1, 4.3 Hz, 1H). |
| | N-(4-((R)-7-amino-5- | | |
| | azaspiro[2.4]heptan-5-yl)-2- | | |
| | methyl-1-((S)-tetrahydrofuran-3- | | |
| | yl)-1H-benzo[d]imidazol-5-yl)-1- | | |
| | (2,6-difluorophenyl)-6-oxo-1,6- | | |
| | dihydropyridazine-3- | | |
| | carboxamide | | |
| Example 153 | 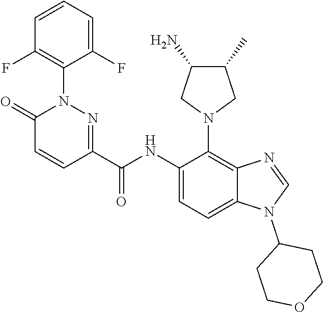 [SMILES: C[C@@H]1CN(c2c(NC(=O)c3ccc(=O)n(-c4c(F)cccc4F)n3)ccc3c2ncn3C2CCOCC2)C[C@@H]1N] | HCOOH Salt | LC-MS m/z: 550.6 (M + 1)⁺ ¹H NMR (400 MHz, CD₃OD) δ 8.36 (d, J = 8.8 Hz, 1H), 8.29-8.19 (m, 2H), 7.71 (ddd, J = 14.7, 8.6, 6.2 Hz, 1H), 7.50 (d, J = 8.9 Hz, 1H), 7.34 (t, J = 9.2 Hz, 3H), 4.21-4.07 (m, 2H), 3.83-3.63 (m, 3H), 3.47- 3.36 (m, 2H), 3.19 (ddd, J = 18.8, 8.0, 5.2 Hz, 3H), 2.23 (ddd, J = 19.2, 13.3, 7.3 Hz, 3H), 2.12 (d, J = 9.6 Hz, 2H), 0.98 (d, J = 7.0 Hz, 3H). |
| | N-(4- | | |
| | ((3R,4R)-3-amino-4- | | |
| | methylpyrrolidin-1-yl)-1- | | |
| | (tetrahydro-2H-pyran-4-yl)-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 154 | 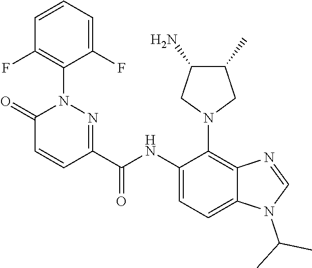 [SMILES: CC(C)n1cnc2c(N3C[C@@H](C)[C@@H](N)C3)c(NC(=O)c3ccc(=O)n(-c4c(F)cccc4F)n3)ccc21] | HCl Salt | LC-MS m/z: 508.5 (M + 1)⁺ ¹H NMR (400 MHz, CD₃OD) δ 8.22 (t, J = 11.4 Hz, 2H), 7.74-7.65 (m, 1H), 7.63 (t, J = 10.0 Hz, 1H), 7.42- 7.28 (m, 3H), 3.86 (d, J = 5.9 Hz, 2H), 3.55-3.41 (m, 2H), 2.56 (s, 1H), 1.68 (d, J = 6.7 Hz, 6H), 1.19 (d, J = 7.1 Hz, 3H). |
| | N-(4-((3R,4R)-3-amino-4- | | |
| | methylpyrrolidin-1-yl)-1- | | |
| | isopropyl-1H-benzo[d]imidazol- | | |
| | 5-yl)-1-(2,6-difluorophenyl)-6- | | |
| | oxo-1,6-dihydropyridazine-3- | | |
| | carboxamide | | |
| Example 155 | 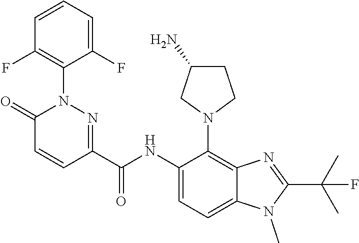 [SMILES: Cn1c(C(C)(C)F)nc2c(N3CC[C@@H](N)C3)c(NC(=O)c3ccc(=O)n(-c4c(F)cccc4F)n3)ccc21] | HCl Salt | LC-MS m/z: 526.5 (M + 1)⁺. ¹H NMR (400 MHz, CD₃OD) δ 8.36 (d, J = 8.9 Hz, 1H), 8.24 (d, J = 9.8 Hz, 1H), 7.69 (tt, J = 8.7, 6.2 Hz, 1H), 7.47 (d, J = 8.9 Hz, 1H), 7.34 (dd, J = 9.0, 7.7 Hz, 3H), 4.00 (t, J = 9.4 Hz, 3H), 3.97 (s, 1H), 3.73 (dd, J = 12.2, 5.2 Hz, 1H), 3.53 (td, J = 10.1, 7.2 Hz, 2H), 3.41-3.34 (m, 1H), 2.24-2.16 (m, 2H), 1.95 (d, J = 2.7 Hz, 3H), 1.90 (d, J = 2.6 Hz, 3H). |
| | (R)-N- | | |
| | (4-(3-aminopyrrolidin-1-yl)-2- | | |
| | fluoropropan-2-yl)-1-methyl-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 156 | 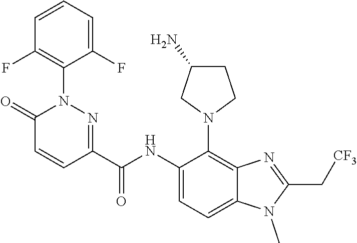 [SMILES: Cn1c(CC(F)(F)F)nc2c(N3CC[C@@H](N)C3)c(NC(=O)c3ccc(=O)n(-c4c(F)cccc4F)n3)ccc21] | HCl Salt | LC-MS m/z: 548.5 (M + 1)⁺. ¹H NMR (400 MHz, CD₃OD) δ 8.37 (d, J = 8.7 Hz, 1H), 8.24 (d, J = 9.8 Hz, 1H), 7.69 (tt, J = 8.5, 6.2 Hz, 1H), 7.52 (d, J = 8.9 Hz, 1H), 7.34 (dd, J = 9.0, 6.7 Hz, 3H), 4.25- 4.07 (m, 2H), 3.95 (s, 1H), 3.72 (dd, J = 12.4, 5.4 Hz, 1H), 3.57 (d, J = 7.4 Hz, 1H), 3.50 (dd, J = 6.9, 5.2 Hz, 1H), 3.38 (dd, J = 15.2, 8.3 Hz, 1H), 2.26-2.13 (m, 2H). |
| | (R)-N-(4-(3-aminopyrrolidin-1- | | |
| | yl)-1-methyl-2-(2,2,2- | | |
| | trifluoroethyl)-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 157 | 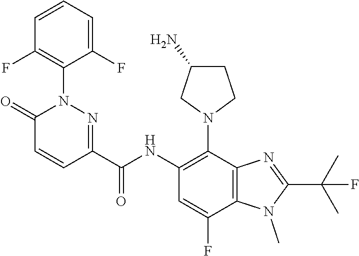 [SMILES: Cn1c(C(C)(C)F)nc2c(N3CC[C@@H](N)C3)c(NC(=O)c3ccc(=O)n(-c4c(F)cccc4F)n3)cc(F)c21] | HCOOH Salt | LC-MS m/z: 544.3 (M + 1)⁺. ¹H NMR (400 MHz, CD₃OD) δ 8.25- 8.13 (m, 2H), 7.70 (tt, J = 8.6, 6.2 Hz, 1H), 7.45-7.21 (m, 3H), 4.16 (t, J = 1.2 Hz, 3H), 3.87 (dp, J = 5.5, 2.7 Hz, 1H), 3.65 (dd, J = 11.7, 5.4 Hz, 1H), 3.47 (td, J = 9.5, 7.3 Hz, 1H), 3.37 (dd, J = 11.7, 2.3 Hz, 1H), 3.32-3.28 (m, 1H), 2.12 (ddd, J = 10.0, 7.3, 4.7 Hz, 2H), 1.95 (d, J = 2.6 Hz, 3H), 1.89 (d, J = 2.5 Hz, 3H). |
| | (R)-N-(4-(3-aminopyrrolidin-1- | | |
| | yl)-7-fluoro-2-(2-fluoropropan-2- | | |
| | yl)-1-methyl-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 158 | 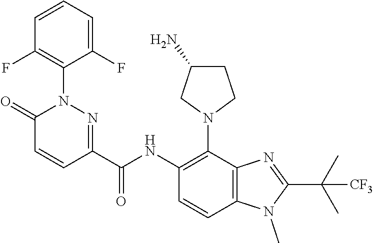 [SMILES: Cn1c(C(C)(C)C(F)(F)F)nc2c(N3CC[C@@H](N)C3)c(NC(=O)c3ccc(=O)n(-c4c(F)cccc4F)n3)ccc21] | HCOOH Salt | LC-MS m/z: 576.2 (M + 1)⁺. ¹H NMR (400 MHz, CD₃OD) δ 8.34 (d, J = 8.9 Hz, 1H), 8.21 (d, J = 9.9 Hz, 1H), 7.69 (tt, J = 8.6, 6.2 Hz, 1H), 7.44 (d, J = 8.9 Hz, 1H), 7.39- 7.23 (m, 3H), 4.03 (s, 3H), 3.92 (dq, J = 5.3, 2.7 Hz, 1H), 3.76 (dd, J = 11.9, 5.2 Hz, 1H), 3.61 (td, J = 9.6, 7.7 Hz, 1H), 3.52-3.36 (m, 2H), 2.17 (ddd, J = 9.6, 6.9, 4.9 Hz, 2H), 1.85 (d, J = 2.4 Hz, 6H). |
| | (R)-N-(4-(3-aminopyrrolidin-1- | | |
| | yl)-1-methyl-2-(1,1,1-trifluoro-2- | | |
| | methylpropan-2-yl)-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 159 | 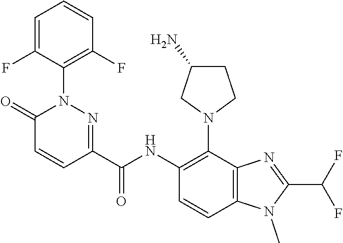 [SMILES: Cn1c(C(F)F)nc2c(N3CC[C@@H](N)C3)c(NC(=O)c3ccc(=O)n(-c4c(F)cccc4F)n3)ccc21] | HCOOH Salt | LC-MS m/z: 516.2 (M + 1)⁺. ¹H NMR (400 MHz, CD₃OD) δ 8.30 (d, 1H), 8.21 (d, 1H), 7.68-7.64 (m, 1H), 7.52 (d, 1H), 7.33-7.29 (m, 3H), 7.06 (d 1H), 4.00- (s, 3H), 3.75 (s, 1H), 3.58 (m, 1H), 3.55- 3.40 (m, 2H), 3.35-3.38 (m, 1H), 2.24-2.14 (m, 2H). |
| | (R)-N- | | |
| | (4-(3-aminopyrrolidin-1-yl)-2- | | |
| | (difluoromethyl)-1-methyl-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 160 | 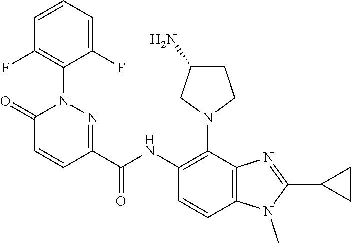 [SMILES: Cn1c(C2CC2)nc2c(N3CC[C@@H](N)C3)c(NC(=O)c3ccc(=O)n(-c4c(F)cccc4F)n3)ccc21] | HCl Salt | LC-MS m/z: 506.2 (M + 1)⁺. ¹H NMR (400 MHz, CD₃OD) δ 8.10 (d, J = 9.9 Hz, 1H), 8.00 (d, J = 8.9 Hz, 1H), 7.63-7.47 (m, 2H), 7.26- 7.17 (m, 3H), 3.96 (s, 3H), 3.76- 3.53 (m, 3H), 3.32 (dd, J = 10.4, 3.2 Hz, 1H), 2.34 (s, 1H), 2.16- 1.99 (m, 2H), 1.57-1.27 (m, 4H). |
| | (R)-N-(4-(3-aminopyrrolidin-1- | | |
| | yl)-2-cyclopropyl-1-methyl-1H- | | |
| | benzo[d]imidazol-5-yl)-1-(2,6- | | |
| | difluorophenyl)-6-oxo-1,6- | | |
| | dihydropyridazine-3- | | |
| | carboxamide | | |
| Example 161 | 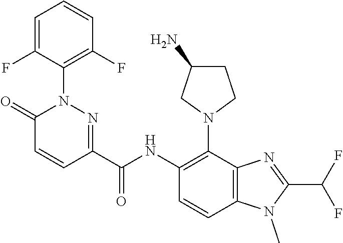 [SMILES: Cn1c(C(F)F)nc2c(N3CC[C@H](N)C3)c(NC(=O)c3ccc(=O)n(-c4c(F)cccc4F)n3)ccc21] | HCl Salt | LC-MS m/z: 516.2 (M + 1)⁺. ¹H NMR (400 MHz, CD₃OD) δ 8.22 (m, 1H), 8.19 (m, 1H), 7.56 (m, 1H), 7.3 (m, 1H), 7.29-7.22 (m, 3H), 7.09 (t, 1H), 3.99 (s, 3H), 3.95 (m, 1H), 3.85 (m, 1H), 3.67 (m, 2H), 3.48 (m, 1H), 2.26-2.21 (m, 2H). |
| | (S)-N-(4-(3-aminopyrrolidin-1- | | |
| | yl)-2-(difluoromethyl)-1-methyl- | | |
| | 1H-benzo[d]imidazol-5-yl)-1- | | |
| | (2,6-difluorophenyl)-6-oxo-1,6- | | |
| | dihydropyridazine-3- | | |
| | carboxamide | | |
| Example 162 | 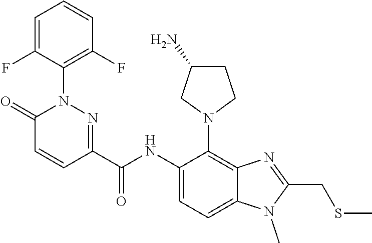 [SMILES: CSCc1nc2c(N3CC[C@@H](N)C3)c(NC(=O)c3ccc(=O)n(-c4c(F)cccc4F)n3)ccc2n1C] | HCl Salt | LC-MS m/z: 526.2 (M + 1)⁺. ¹H NMR (400 MHz, CD₃OD) δ 8.29- 8.20 (m, 2H), 7.76-7.64 (m, 1H), 7.63-7.51 (m, 1H), 7.37-7.30 (m, 3H), 4.23 (br s, 2H), 4.01-3.94 (m, 3H), 3.94-3.89 (m, 1H), 3.66 (br s, 2H), 3.53-3.47 (m, 1H), 3.31- 3.13 (m, 1H), 2.24-2.18 (m, 5H). |
| | (R)-N- | | |
| | (4-(3-aminopyrrolidin-1-yl)-1- | | |
| | methyl-2-((methylthio)methyl)- | | |
| | 1H-benzo[d]imidazol-5-yl)-1- | | |
| | (2,6-difluorophenyl)-6-oxo-1,6- | | |
| | dihydropyridazine-3- | | |
| | carboxamide | | |
| Example 163 | 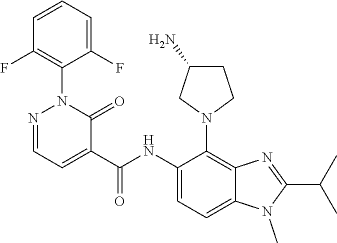 [SMILES: CC(C)c1nc2c(N3CC[C@@H](N)C3)c(NC(=O)c3ccnn(-c4c(F)cccc4F)c3=O)ccc2n1C] | HCl Salt | LC-MS m/z: 508.2 (M + 1)⁺. ¹H NMR (400 MHz, CD₃OD) δ 8.62 (s, 1H), 8.36 (m, 2H), 7.55 (m, 2H), 7.21 (m, 2H), 3.93 (m, 1H), 3.85 (m, 2H), 3.81 (m, 2H), 3.39 (m, 2H), 3.22 (m, 1H), 2.48 (s, 1H), 2.05 (m, 1H), 1.42 (s, 6H). |
| | (R)-N- | | |
| | (4-(3-aminopyrrolidin-1-yl)-2- | | |
| | isopropyl-1-methyl-1H- | | |
| | benzo[d]imidazol-5-yl)-2-(2,6- | | |
| | difluorophenyl)-3-oxo-2,3- | | |
| | dihydropyridazine-4- | | |
| | carboxamide | | |
| Example 164 | 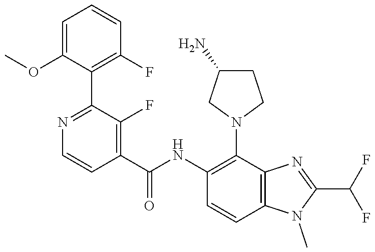 [SMILES: COc1cccc(F)c1-c1nccc(C(=O)Nc2ccc3c(nc(C(F)F)n3C)c2N2CC[C@@H](N)C2)c1F] | HCl Salt | LC-MS m/z: 529.2 (M + 1)⁺. ¹H NMR (400 MHz, CD₃OD) δ 8.48 (s, 1H), 7.66-7.61 (m, 1H), 7.46 (m, 1H), 7.33 (m, 1H), 7.21 (m, 1H), 7.08 (t, 1H), 3.87 (m, 6H), 3.77 (m, 1H), 3.64 (m, 1H), 3.58- 3.46 (m, 2H), 3.33 (m, 1H), 2.06 (m, 1H), 1.84 (m, 1H). |
| | N-(4- | | |
| | ((S)-3-aminopyrrolidin-1-yl)-2- | | |
| | (difluoromethyl)-1-methyl-1H- | | |
| | benzo[d]imidazol-5-yl)-3-fluoro- | | |
| | 2-(2-fluoro-6- | | |
| | methoxyphenyl)isonicotinamide | | |
| Example 165 | 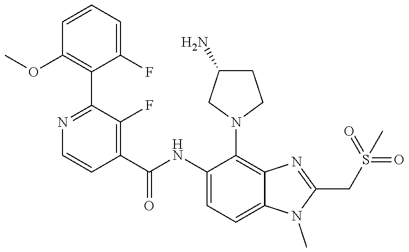 [SMILES: COc1cccc(F)c1-c1nccc(C(=O)Nc2ccc3c(nc(CS(C)(=O)=O)n3C)c2N2CC[C@@H](N)C2)c1F] | HCl Salt | LC-MS m/z: 571.2 (M + 1)⁺. ¹H NMR (400 MHz, CD₃OD) δ 8.74 (d, J = 5.0 Hz, 1H), 8.09 (br t, J = 4.8 Hz, 2H), 7.72 (br d, J = 8.7 Hz, 1H), 7.64-7.54 (m, 1H), 7.06 (d, J = 8.4 Hz, 1H), 6.94 (t, J = 8.8 Hz, 1H), 5.31-5.21 (m, 1H), 5.32- 5.21 (m, 1H), 4.86-4.81 (m, 1H), 4.09 (s, 4H), 3.85 (s, 4H), 3.64 (br d, J = 3.8 Hz, 2H), 3.39-3.33 (m, 1H), 3.30-3.24 (m, 3H), 2.66 (s, 1H), 2.60-2.45 (m, 1H), 2.36- 2.27 (m, 1H). |
| | N-(4- | | |
| | ((R)-3-aminopyrrolidin-1-yl)-1- | | |
| | methyl-2- | | |
| | ((methylsulfonyl)methyl)-1H- | | |
| | benzo[d]imidazol-5-yl)-3-fluoro- | | |
| | 2-(2-fluoro-6- | | |
| | methoxyphenyl)isonicotinamide | | |

### Example 166: N-[4-[(3R,4R)-3-amino-4-methyl-pyrrolidin-1-yl]-1-methyl-indazol-5-yl]-1-(2,6-difluorophenyl)-6-oxo-pyridazine-3-carboxamide

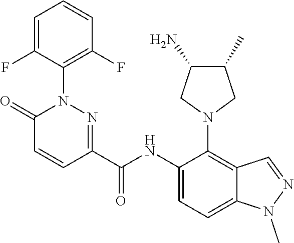 [SMILES: C[C@@H]1CN(c2c(NC(=O)c3ccc(=O)n(-c4c(F)cccc4F)n3)ccc3c2cnn3C)C[C@@H]1N]

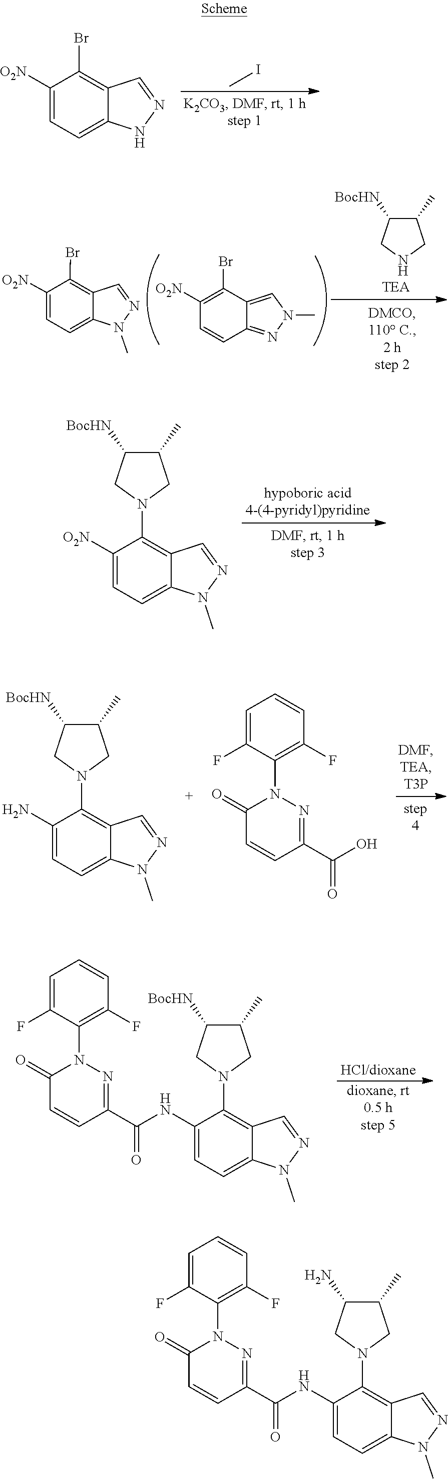

### Step 1: 4-bromo-1-methyl-5-nitro-indazole

To a solution of 4-bromo-5-nitro-1H-indazole (500 mg, 2.07 mmol, 1 eq) in DMF (2 mL) was added K₂CO₃ (856.54 mg, 6.20 mmol, 3 eq) and Mel (439.84 mg, 3.10 mmol, 192.91 uL, 1.5 eq). The mixture was stirred at 20° C. for 1 hr. LCMS showed the starting material was consumed, the desired compound was observed. The reaction mixture was poured into water (50 ml), and extracted with EtOAc (40 mL×3). The combined organic layers were washed with brine, dried over Na₂SO₄, filtered and concentrated under reduced pressure to give a residue. The residue was purified by flash silica gel chromatography (ISCO®; 10 g SepaFlash® Silica Flash Column, Eluent of 0^{˜}50% Ethyl acetate/Petroleum ether gradient). Compound 4-bromo-1-methyl-5-nitro-indazole (205 mg, Yield: 38.75%) was obtained as a yellow solid.

LC-MS m/z: 258.1 (M+1)⁺

### Step 2: tert-butyl N-[(3R,4R)-4-methyl-1-(1-methyl-5-nitro-indazol-4-yl)pyrrolidin-3-yl]carbamate

To a solution of tert-butyl N-[(3R,4R)-4-methylpyrrolidin-3-yl]carbamate (78.22 mg, 390.54 umol, 1 eq) in DMSO (3 mL) was added TEA (118.55 mg, 1.17 mmol, 163.07 uL, 3 eq) and 4-bromo-1-methyl-5-nitro-indazole (100 mg, 390.54 umol, 1 eq). The mixture was stirred at 110° C. for 2 hr. LCMS showed the starting material was consumed up and the desired compound was observed. The reaction mixture was poured into water (50 ml), and extracted with EtOAc (40 mL×3). The combined organic layers were washed with brine, dried over Na₂SO₄, filtered and concentrated under reduced pressure to give a residue. The crude product was used into the next step without further purification. Compound tert-butyl N-[(3R,4R)-4-methyl-1-(1-methyl-5-nitro-indazol-4-yl)pyrrolidin-3-yl]carbamate (140 mg, Yield: 95.49%) was obtained as a yellow oil.

LC-MS m/z: 376.3 (M+1)⁺.

### Step 3: tert-butyl N-[(3R,4R)-1-(5-amino-1-methyl-indazol-4-yl)-4-methyl-pyrrolidin-3-yl]carbamate

To a solution of tert-butyl N-[(3R,4R)-4-methyl-1-(1-methyl-5-nitro-indazol-4-yl) pyrrolidin-3-yl]carbamate (130 mg, 346.28 umol, 1 eq) in DMF (2 mL) was added hypoboric acid (93.13 mg, 1.04 mmol, 3 eq) and 4-(4-pyridyl)pyridine (5.41 mg, 34.63 umol, 0.1 eq). The mixture was stirred at 20° C. for 0.5 hr. LCMS showed the starting material was consumed, the desired compound was observed. The reaction mixture was poured into water (50 ml), and extracted with EtOAc (40 mL×3). The combined organic layers were washed with brine, dried over Na₂SO₄, filtered and concentrated under reduced pressure to give a residue. The residue was used next step without further purification. Compound tert-butyl N-[(3R,4R)-1-(5-amino-1-methyl-indazol-4-yl)-4-methyl-pyrrolidin-3-yl]carbamate (100 mg, Yield: 83.60%) was obtained as a yellow oil.

LC-MS m/z: 346.3 (M+1)⁺.

### Step 4: tert-butyl N-[(3R,4R)-1-[5-[[1-(2,6-difluorophenyl)-6-oxo-pyridazine-3-carbonyl]amino]-1-methyl-indazol-4-yl]-4-methyl-pyrrolidin-3-yl]carbamate

To a solution of tert-butyl N-[(3R,4R)-1-(5-amino-1-methyl-indazol-4-yl)-4-methyl-pyrrolidin-3-yl]carbamate (100 mg, 289.49 umol, 1 eq) in DMF (3 mL) was added T₃P (552.66 mg, 868.46 umol, 516.50 uL, 50% purity, 3 eq) and 1-(2,6-difluorophenyl)-6-oxo-pyridazine-3-carboxylic acid (73.00 mg, 289.49 umol, 1 eq) and TEA (146.46 mg, 1.45 mmol, 201.46 uL, 5 eq). The mixture was stirred at 20° C. for 1 hr. LCMS showed the most of starting material was consumed, the desired compound was observed. The reaction mixture was poured into water (80 ml), and extracted with EtOAc (40 mL×3). The combined organic layers were washed with brine, dried over Na₂SO₄, filtered and concentrated under reduced pressure to give a residue. The residue was used next step without further purification. Compound tert-butyl N-[(3R,4R)-1-[5-[[1-(2,6-difluorophenyl)-6-oxo-pyridazine-3-carbonyl]amino]-1-methyl-indazol-4-yl]-4-methyl-pyrrolidin-3-yl]carbamate (120 mg, Yield: 71.52%) was obtained as a brown oil.

LC-MS m/z: 580.4 (M+1)⁺.

### Step 5: N-[4-[(3R,4R)-3-amino-4-methyl-pyrrolidin-1-yl]-1-methyl-indazol-5-yl]-1-(2,6-difluoro phenyl)-6-oxo-pyridazine-3-carboxamide

To a solution of tert-butyl N-[(3R,4R)-1-[5-[[1-(2,6-difluorophenyl)-6-oxo-pyridazine-3-carbonyl]amino]-1-methyl-indazol-4-yl]-4-methyl-pyrrolidin-3-yl]carbamate (120 mg, 207.04 umol, 1 eq) in dioxane (0.5 mL) was added HCl/dioxane (4 M, 1 mL, 19.32 eq). The mixture was stirred at 20° C. for 0.5 hr. LCMS showed the starting material was consumed, the desired compound was observed. The reaction mixture was concentrated in vacuum. The residue was purified by prep-HPLC (Boston Green ODS 150×30 mm×5 μm; mobile phase: [water (HCl)-ACN]; B %: 23%-43%, 10 min). Example 166 (13.22 mg, Yield: 13.32%) was obtained as a brown solid.

¹H NMR (400 MHz, CD₃OD) δ 8.33-8.14 (m, 2H), 7.72-7.54 (m, 1H), 7.73-7.53 (m, 1H), 7.74-7.51 (m, 1H), 7.37-7.22 (m, 1H), 7.38-7.21 (m, 1H), 7.13 (d, J=8.9 Hz, 1H), 7.34-7.06 (m, 1H), 4.90 (s, 21H), 4.10 (dd, J=5.9, 10.7 Hz, 1H), 4.02 (s, 3H), 3.84 (br d, J=3.3 Hz, 1H), 3.70 (br t, J=8.5 Hz, 1H), 3.61 (br dd, J=3.0, 10.7 Hz, 1H), 3.51 (br t, J=9.0 Hz, 1H), 2.68-2.55 (m, 1H), 1.17-1.11 (m, 1H), 1.17-1.11 (m, 1H), 1.15 (d, J=7.0 Hz, 1H).

LC-MS m/z: 480.1 (M+1)+.

| No. | Structure | Salt form | MS and 1HNMR |
|---|---|---|---|
| Example 167 | 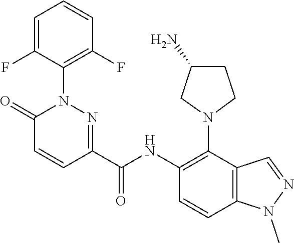 [SMILES: Cn1ncc2c(N3CC[C@@H](N)C3)c(NC(=O)c3ccc(=O)n(-c4c(F)cccc4F)n3)ccc21] | HCl Salt | LC-MS m/z: 466.2 (M + 1)⁺. ¹H NMR (400 MHz, CD₃OD) δ 8.23 (s, 2 H), 7.77 (d, J = 8.82 Hz, 1 H), 7.63-7.73 (m, 1 H), 7.28-7.40 (m, 3 H), 7.22 (d, J = 8.94 Hz, 1 H), 4.05 (s, 3 H), 3.86-3.95 (m, 1 H), 3.69- 3.85 (m, 2 H), 3.41-3.56 (m, 2 H), 2.20-2.43 (m, 1 H), 1.88-2.09 (m, 1 H). |
| | (R)-N-(4-(3-aminopyrrolidin-1- | | |
| | yl)-1-methyl-1H-indazol-5-yl)-1- | | |
| | (2,6-difluorophenyl)-6-oxo-1,6- | | |
| | dihydropyridazine-3- | | |
| | carboxamide | | |
| Example 168 | 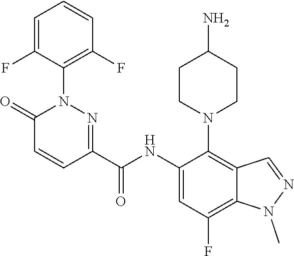 [SMILES: Cn1ncc2c(N3CCC(N)CC3)c(NC(=O)c3ccc(=O)n(-c4c(F)cccc4F)n3)cc(F)c21] | HCl Salt | LC-MS m/z: 498.3 (M + 1)⁺. 1H NMR (400 MHz, CD₃OD) δ 8.29 (d, J = 2.1 Hz, 1H), 8.23 (d, J = 9.9 Hz, 1H), 8.14 (d, J = 13.2 Hz, 1H), 7.74- 7.64 (m, 1H), 7.38-7.29 (m, 3H), 4.20 (s, 3H), 3.30-3.20 (m, 3H), 3.15 (br s, 2H), 1.92 (br d, J = 9.4 Hz, 2H), 1.68 (br d, J = 9.3 Hz, 2H). |
| | N-(4-(4-aminopiperidin-1-yl)-7- | | |
| | fluoro-1-methyl-1H-indazol-5- | | |
| | yl)-1-(2,6-difluorophenyl)-6-oxo- | | |
| | 1,6-dihydropyridazine-3- | | |
| | carboxamide | | |
| Example 169 | 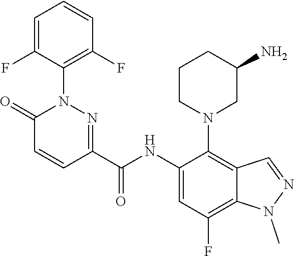 [SMILES: Cn1ncc2c(N3CCC[C@@H](N)C3)c(NC(=O)c3ccc(=O)n(-c4c(F)cccc4F)n3)cc(F)c21] | HCl Salt | LC-MS m/z: 498.3 (M + 1)⁺. ¹H NMR (400 MHz, CD₃OD) δ 8.36 (br d, J = 12.9 Hz, 1H), 8.24 (d, J = 9.9 Hz, 1H), 8.18 (d, J = 2.3 Hz, 1H), 7.78- 7.68 (m, 1H), 7.42-7.30 (m, 3H), 4.21 (s, 3H), 3.24-3.06 (m, 3H), 3.04-2.88 (m, 2H), 1.87 (br d, J = 9.3 Hz, 1H), 1.76 (br d, J = 13.7 Hz, 1H), 1.50 (dq, J = 3.5, 12.2 Hz, 1H), 1.39-1.29 (m, 1H). |
| | (R)-N-(4-(3-aminopiperidin-1-yl)- | | |
| | 7-fluoro-1-methyl-1H-indazol-5- | | |
| | yl)-1-(2,6-difluorophenyl)-6-oxo- | | |
| | 1,6-dihydropyridazine-3- | | |
| | carboxamide | | |
| Example 170 | 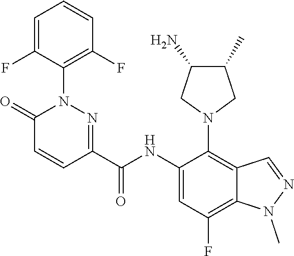 [SMILES: C[C@@H]1CN(c2c(NC(=O)c3ccc(=O)n(-c4c(F)cccc4F)n3)cc(F)c3c2cnn3C)C[C@@H]1N] | HCl Salt | LC-MS m/z: 498.3 (M + 1)⁺. ¹H NMR (400 MHz, CD₃OD) δ 8.28- 8.11 (m, 2H), 7.89 (d, J = 13.0 Hz, 1H), 7.76-7.59 (m, 1H), 7.39-7.24 (m, 3H), 4.20 (s, 3H), 3.91-3.83 (m, 1H), 3.83-3.76 (m, 1H), 3.47 (dd, J = 7.2, 9.2 Hz, 1H), 3.37 (dd, J = 4.6, 10.3 Hz, 1H), 3.24 (t, J = 8.6 Hz, 1H), 2.49 (td, J = 7.3, 14.6 Hz, 1H), 1.08 (d, J = 7.2 Hz, 3H). |
| | N-(4-((3R,4R)-3-amino-4- | | |
| | methylpyrrolidin-1-yl)-7-fluoro- | | |
| | 1-methyl-1H-indazol-5-yl)-1- | | |
| | (2,6-difluorophenyl)-6-oxo-1,6- | | |
| | dihydropyridazine-3- | | |
| | carboxamide | | |
| Example 171 | 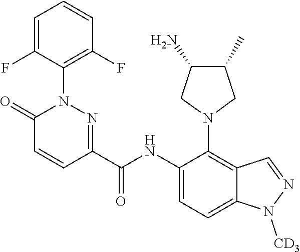 [SMILES: [2H]C([2H])([2H])n1ncc2c(N3C[C@@H](C)[C@@H](N)C3)c(NC(=O)c3ccc(=O)n(-c4c(F)cccc4F)n3)ccc21] | HCl Salt | LC-MS m/z: 483.3 (M + 1)⁺. ¹H NMR (400 MHz, CD₃OD) δ 8.28- 8.13 (m, 2H), 7.74-7.58 (m, 2H), 7.37-7.24 (m, 3H), 7.10 (d, J = 9.1 Hz, 1H), 4.04 (dd, J = 5.8, 10.3 Hz, 1H), 3.80-3.61 (m, 2H), 3.57-3.42 (m, 2H), 2.53 (quind, J = 7.1, 14.1 Hz, 1H), 1.11 (d, J = 7.0 Hz, 3H). |
| | N-(4-((3R,4R)-3-amino-4- | | |
| | methylpyrrolidin-1-yl)-1- | | |
| | (methyl-d3)-1H-indazol-5-yl)-1- | | |
| | (2,6-difluorophenyl)-6-oxo-1,6- | | |
| | dihydropyridazine-3- | | |
| | carboxamide | | |
| Example 172 | 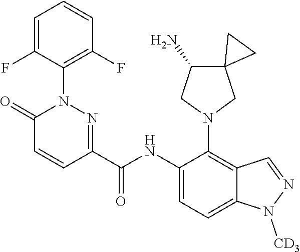 [SMILES: [2H]C([2H])([2H])n1ncc2c(N3C[C@H](N)C4(CC4)C3)c(NC(=O)c3ccc(=O)n(-c4c(F)cccc4F)n3)ccc21] | HCl Salt | LC-MS m/z: 495.2 (M + 1)⁺. ¹H NMR (400 MHz, CD₃OD) δ 8.25- 8.19 (m, 2H), 7.70-7.61 (m, 2H), 7.34-7.25 (m, 3H), 7.21-7.14 (m, 1H), 4.30-4.20 (m, 1H), 4.09-4.00 (m, 1H), 3.74-3.63 (m, 1H), 3.17- 3.10 (m, 2H), 0.94 (s, 1H), 0.87- 0.79 (m, 1H), 0.77-0.67 (m, 2H) |
| | (R)-N-(4-(7-amino-5- | | |
| | azaspiro[2.4]heptan-5-yl)-1- | | |
| | (methyl-d3)-1H-indazol-5-yl)-1- | | |
| | (2,6-difluorophenyl)-6-oxo-1,6- | | |
| | dihydropyridazine-3- | | |
| | carboxamide | | |
| Example 173 | 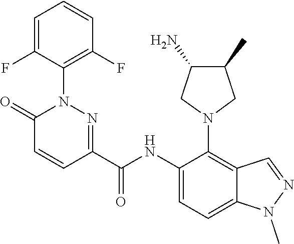 [SMILES: C[C@H]1CN(c2c(NC(=O)c3ccc(=O)n(-c4c(F)cccc4F)n3)ccc3c2cnn3C)C[C@@H]1N] | HCl Salt | LC-MS m/z: 480.2 (M + 1)⁺. ¹H NMR (500 MHz, CD₃OD) δ 8.26- 8.21 (m, 2H), 7.88-7.84 (m, 1H), 7.72-7.64 (m, 1H), 7.35-7.23 (m, 4H), 4.07-4.04 (m, 3H), 3.97-3.89 (m, 2H), 3.57-3.50 (m, 1H), 3.38- 3.36 (m, 1H), 3.20-3.15 (m, 1H), 2.40-2.30 (m, 1H), 1.12-1.08 (m, 3H) |
| | N-(4-((3R,4S)-3-amino-4- | | |
| | methylpyrrolidin-1-yl)-1-methyl- | | |
| | 1H-indazol-5-yl)-1-(2,6- | | |
| | difluorophenyl)-6-oxo-1,6- | | |
| | dihydropyridazine-3- | | |
| | carboxamide | | |
| Example 174 | 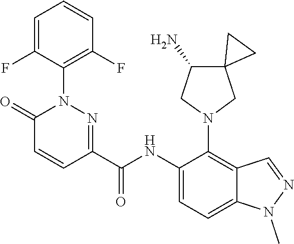 [SMILES: Cn1ncc2c(N3C[C@H](N)C4(CC4)C3)c(NC(=O)c3ccc(=O)n(-c4c(F)cccc4F)n3)ccc21] | HCOOH Salt | LC-MS m/z: 492.5 (M + 1)⁺. ¹H NMR (400 MHz, CD₃OD) δ 8.23 (d, J = 10.3 Hz, 2H), 7.77-7.58 (m, 2H), 7.31 (dd, J = 10.2, 7.0 Hz, 3H), 7.20 (d, J = 8.9 Hz, 1H), 4.23 (dd, J = 10.5, 5.4 Hz, 1H), 4.04 (s, 3H), 3.67 (dd, J = 10.5, 2.6 Hz, 1H), 3.15 (d, J = 9.2 Hz, 1H), 0.99 (dt, J = 10.4, 5.5 Hz, 1H), 0.87-0.76 (m, 1H), 0.72 (ddd, J = 11.8, 9.4, 5.1 Hz, 2H). |
| | (R)-N-(4-(7-amino-5- | | |
| | azaspiro[2.4]heptan-5-yl)-1- | | |
| | methyl-1H-indazol-5-yl)-1-(2,6- | | |
| | difluorophenyl)-6-oxo-1,6- | | |
| | dihydropyridazine-3- | | |
| | carboxamide | | |

### Example 175: (R)—N-(4-(3-aminopyrrolidin-1-yl)-2-methyl-2H-indazol-5-yl)-1-(2,6-difluorophenyl)-6-oxo-1,6-dihydropyridazine-3-carboxamide

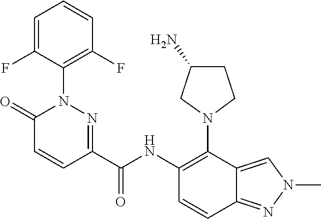 [SMILES: Cn1cc2c(N3CC[C@@H](N)C3)c(NC(=O)c3ccc(=O)n(-c4c(F)cccc4F)n3)ccc2n1]

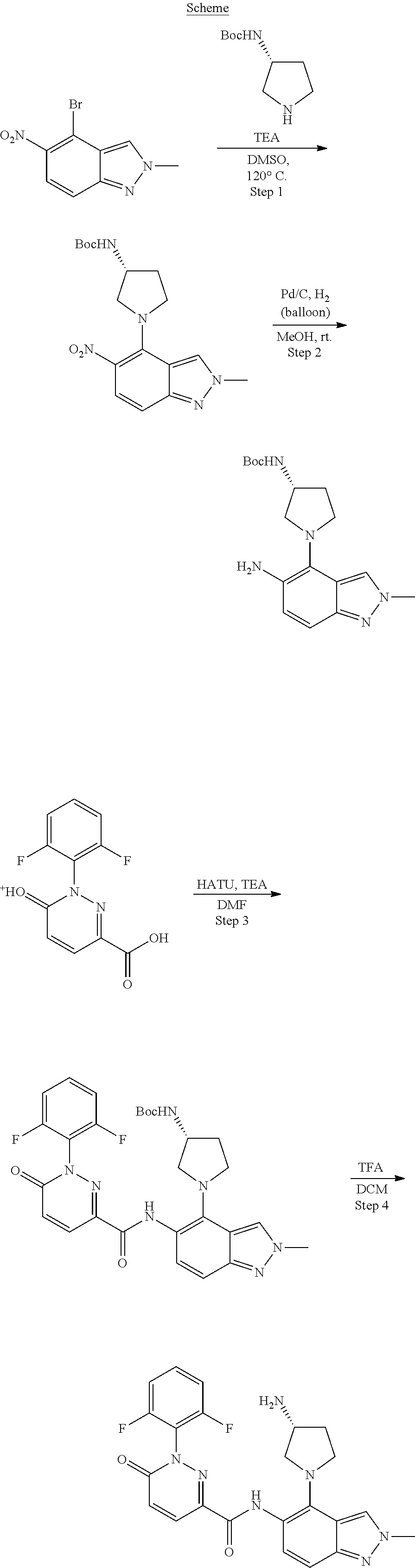

### Step 1: (R)-tert-butyl (1-(2-methyl-5-nitro-2H-indazol-4-yl)pyrrolidin-3-yl)carbamate

To a solution of 4-bromo-2-methyl-5-nitro-indazole (300 mg, 1.17 mmol, 1 eq) in DMSO (6 mL) were added tert-butyl N-[(3R)-pyrrolidin-3-yl]carbamate (327.32 mg, 1.76 mmol, 1.5 eq) and TEA (237.11 mg, 2.34 mmol, 326.15 uL, 2 eq). The mixture was stirred at 110° C. for 2 hr. LCMS showed desired mass formed. TLC indicated new spot produced. The reaction mixture was quenched by addition water (10 mL) at 25° C. The combined organic layers were extracted with EtOAc 30 mL (10 mL×3), dried over Na₂SO₄, filtered and concentrated under reduced pressure to give a residue. The residue was purified by flash silica gel chromatography (ISCO©; 4 g SepaFlash® Silica Flash Column, Eluent of 0^{˜}60% Ethyl acetate/Petroleum ether gradient). The compound tert-butyl N-[(3R)-1-(2-methyl-5-nitro-indazol-4-yl)pyrrolidin-3-yl]carbamate (400 mg, Yield: 94.47%) was gave as a brown oil.

LC-MS m/z: 362.2 (M+1)⁺.

### Step 2: (R)-tert-butyl (1-(5-amino-2-methyl-2H-indazol-4-yl)pyrrolidin-3-yl)carbamate

To a solution of tert-butyl N-[(3R)-1-(2-methyl-5-nitro-indazol-4-yl)pyrrolidin-3-yl]carbamate (300 mg, 830.12 umol, 1 eq) in MeOH (5 mL) was added Pd/C (87.99 mg, 83.01 umol, 10%, w %, 0.1 eq). The mixture was stirred with a balloon of H₂ at 25° C. for 2 hr. LCMS showed the desired mass formed and starting material was consumed. The reaction mixture was filtered. The filtrate was concentrated in vacuum to give the crude product. The crude product was used directly to next step without further purification. The compound tert-butyl N-[(3R)-1-(5-amino-2-methyl-indazol-4-yl)pyrrolidin-3-yl]carbamate (250 mg, Yield: 90.87%) was obtained as blue solid.

LC-MS m/z: 332.2 (M+1)⁺.

### Step 3: (R)-tert-butyl (1-(5-(1-(2,6-difluorophenyl)-6-oxo-1,6-dihydropyridazine-3-carboxamido)-2-methyl-2H-indazol-4-yl)pyrrolidin-3-yl)carbamate

To a solution of tert-butyl N-[(3R)-1-(5-amino-2-methyl-indazol-4-yl)pyrrolidin-3-yl]carbamate (197.13 mg, 594.83 umol, 1 eq) in DMF (3 mL) were added 1-(2,6-difluorophenyl)-6-oxo-pyridazine-3-carboxylic acid (150 mg, 594.83 umol, 1 eq), TEA (120.38 mg, 1.19 mmol, 165.58 uL, 2 eq) and HATU (339.26 mg, 892.24 umol, 1.5 eq). The mixture was stirred at 25° C. for 2 hr. LCMS show desired mass formed. TLC indicated new spots produced. The reaction mixture was quenched by addition water (15 mL) at 25° C. The combined organic layers were extracted with EtOAc (15 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0^{˜}67% Ethyl acetate/Petroleum ether gradient). The product tert-butyl N-[(3R)-1-[5-[[1-(2,6-difluorophenyl)-6-oxo-pyridazine-3-carbonyl]amino]-2-methyl-indazol-4-yl]pyrrolidin-3-yl]carbamate (250 mg, Yield: 74.31%) was obtained as a brown solid.

LC-MS m/z: 566.2 (M+1)⁺.

### Step 4: (R)—N-(4-(3-aminopyrrolidin-1-yl)-2-methyl-2H-indazol-5-yl)-1-(2,6-difluorophenyl)-6-oxo-1,6-dihydropyridazine-3-carboxamide

To a solution of tert-butyl N-[(3R)-1-[5-[[1-(2,6-difluorophenyl)-6-oxo-pyridazine-3-carbonyl]amino]-2-methyl-indazol-4-yl]pyrrolidin-3-yl]carbamate (250 mg, 442.03 umol, 1 eq) in DCM (2 mL) was added TFA (5.13 g, 45.02 mmol, 3.33 mL, 101.85 eq) and. The mixture was stirred at 25° C. for 2 hr. LCMS show desired mass formed. The combined organic phases were concentrated in vacuum to give the crude product. The crude product was purified by pre-HPLC (Boston Prime C18 150×30 mm×5 um, water (FA)-ACN, Begin B 6, End B 36, Gradient Time (min) 12, 100% B Hold Time (min) 2, FlowRate (ml/min) 25). Example 175 (133.76 mg, Yield: 65.01%) was obtained as a yellow solid.

LC-MS m/z: 466.2 (M+1)⁺.

¹H NMR (500 MHz, CD₃OD) δ 8.44 (s, 1H), 8.22 (d, J=9.92 Hz, 1H), 7.64-7.74 (m, 2H), 7.26-7.38 (m, 4H), 4.17-4.25 (m, 3H), 3.75-3.86 (m, 2H), 3.64-3.71 (m, 1H), 3.37-3.47 (m, 2H), 2.25-2.37 (m, 1H), 1.91-2.01 (in, 1H)

| No. | Structure | Salt form | MS and 1HNMR |
|---|---|---|---|
| Example 176 | 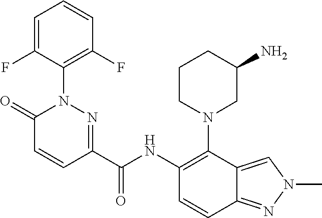 [SMILES: Cn1cc2c(N3CCC[C@@H](N)C3)c(NC(=O)c3ccc(=O)n(-c4c(F)cccc4F)n3)ccc2n1] | HCOOH Salt | LC-MS m/z: 480.5 (M + 1)⁺. ¹H NMR (400 MHz, CD₃OD) δ 8.40 (d, J = 8.4 Hz, 2H), 8.23 (d, J = 9.8 Hz, 1H), 7.74 (tt, J = 8.6, 6.2 Hz, 1H), 7.50 (d, J = 9.3 Hz, 1H), 7.44- 7.27 (m, 3H), 4.21 (s, 3H), 3.25 (dd, J = 10.6, 4.0 Hz, 1H), 3.18-2.97 (m, 3H), 2.92 (dt, J = 11.8, 3.7 Hz, 1H), 1.93-1.83 (m, 1H), 1.75 (dt, J = 13.6, 3.4 Hz, 1H), 1.51 (qd, J = 12.1, 3.9 Hz, 1H), 1.44-1.29 (m, 1H). |
| | (R)-N-(4-(3-aminopiperidin-1-yl)- | | |
| | 2-methyl-2H-indazol-5-yl)-1-(2,6- | | |
| | difluorophenyl)-6-oxo-1,6- | | |
| | dihydropyridazine-3-carboxamide | | |
| Example 177 | 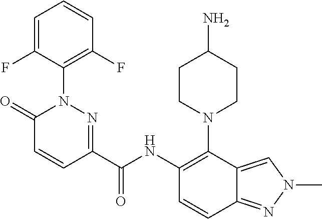 [SMILES: Cn1cc2c(N3CCC(N)CC3)c(NC(=O)c3ccc(=O)n(-c4c(F)cccc4F)n3)ccc2n1] | HCOOH Salt | LC-MS m/z: 480.5 (M + 1)⁺. ¹H NMR (400 MHz, CD₃OD) δ 8.48 (s, 1H), 8.23 (d, J = 9.9 Hz, 1H), 8.10 (d, J = 9.2 Hz, 1H), 7.70 (tt, J = 8.5, 6.2 Hz, 1H), 7.45 (d, J = 9.2 Hz, 1H), 7.39-7.30 (m, 3H), 4.21 (s, 3H), 3.31-3.27 (m, 1H), 3.22 (dq, J = 8.9, 4.3 Hz, 1H), 3.14 (ddd, J = 12.1, 8.7, 3.1 Hz, 2H), 1.95 (dq, J = 13.2, 3.9 Hz, 2H), 1.66 (dtd, J = 12.7, 8.8, 3.6 Hz, 2H). |
| | N-(4-(4-aminopiperidin-1-yl)-2- | | |
| | methyl-2H-indazol-5-yl)-1-(2,6- | | |
| | difluorophenyl)-6-oxo-1,6- | | |
| | dihydropyridazine-3-carboxamide | | |
| Example 178 | 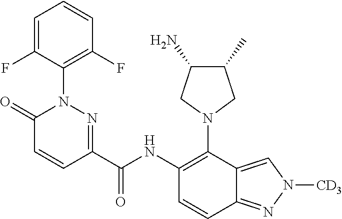 [SMILES: [2H]C([2H])([2H])n1cc2c(N3C[C@@H](C)[C@@H](N)C3)c(NC(=O)c3ccc(=O)n(-c4c(F)cccc4F)n3)ccc2n1] | HCl Salt | LC-MS m/z: 483.2 (M + 1)⁺. ¹H NMR (400 MHz, CD₃OD) δ 8.85- 8.80 (m, 1H), 8.22 (d, J = 9.9 Hz, 1H), 7.73-7.62 (m, 1H), 7.58-7.52 (m, 1H), 7.37-7.27 (m, 3H), 7.19- 7.12 (m, 1H), 4.09 (dd, J = 5.8, 10.8 Hz, 1H), 3.91-3.84 (m, 1H), 3.80- 3.71 (m, 1H), 3.65-3.52 (m, 2H), 2.75-2.61 (m, 1H), 1.19 (d, J = 7.0 Hz, 3H). |
| | N-(4-((3R,4R)-3-amino-4- | | |
| | methylpyrrolidin-1-yl)-2-(methyl- | | |
| | d3)-2H-indazol-5-yl)-1-(2,6- | | |
| | difluorophenyl)-6-oxo-1,6- | | |
| | dihydropyridazine-3-carboxamide | | |

### Example 179: N-[4-[(3R)-3-aminopyrrolidin-1-yl]-1-isopropyl-indazol-5-yl]-1-(2,6-difluorophenyl)-6-oxo-pyridazine-3-carboxamide

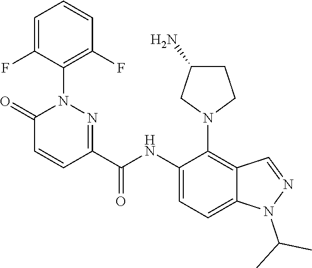 [SMILES: CC(C)n1ncc2c(N3CC[C@@H](N)C3)c(NC(=O)c3ccc(=O)n(-c4c(F)cccc4F)n3)ccc21]

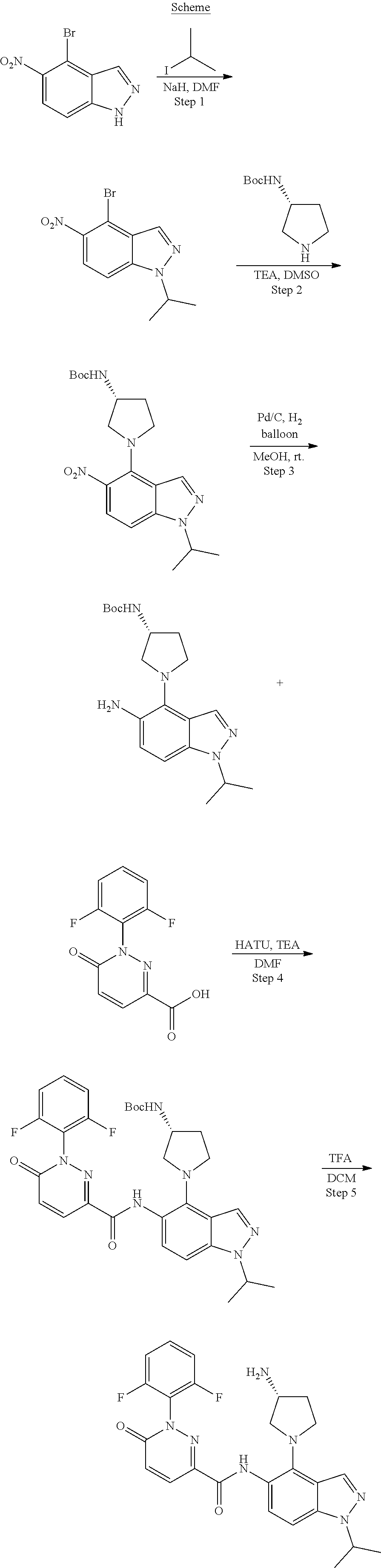

### Step 1: 4-bromo-1-isopropyl-5-nitro-1H-indazole

To a solution of 4-bromo-5-nitro-1H-indazole (5 g, 20.659 mmol) and 2-iodopropane (5.27 g, 30.988 mmol) in DMF (50 mL) was added NaH (1.07 g, 26.856 mmol) in portions at 0^{˜}5° C. The reaction was stirred for 2 hours at rt to give a black brown solution. LCMS showed the starting material was consumed up and two peaks with desired mass was detected. The reaction was quenched with H₂O and extracted with EA (60 mL×3). The combined organic layers were washed with brine, dried over Na₂SO₄, filtered and concentrated to dryness. The residue was purified by flash column (SiO₂, eluted with PE to 5% EA in PE) to afford 4-bromo-5-nitro-1-(prop-2-yl)indazole (2.6 g, Yield: 44.29%) as a yellow solid.

LC-MS m/z: 284.1 (M+1)⁺.

### Step 2: tert-butyl N-[(3R)-1-(1-isopropyl-5-nitro-indazol-4-yl)pyrrolidin-3-yl]carbamate

To a solution of 4-bromo-1-isopropyl-5-nitro-indazole (300 mg, 1.06 mmol, 1 eq) in DMSO (4 mL) were added TEA (213.70 mg, 2.11 mmol, 293.94 uL, 2 eq) and tert-butyl N-[(3R)-pyrrolidin-3-yl]carbamate (295.00 mg, 1.58 mmol, 1.5 eq). The mixture was stirred at 120° C. for 2 hr. TLC showed starting material consumed and new spot formed. LCMS showed the desired mass formed. The reaction mixture was poured into H₂O (40 mL), and extracted with EtOAc (15 mL×3). The combined organic phase dried over anhydrous Na₂SO₄, filtered and concentrated in vacuum. The residue was purified by flash silica gel chromatography (ISCO®; 20 g SepaFlash® Silica Flash Column, eluent of [0^{˜}30]% pet. ether/ethyl acetate gradient). Compound tert-butyl N-[(3R)-1-(1-isopropyl-5-nitro-indazol-4-yl)pyrrolidin-3-yl]carbamate (395 mg, Yield: 96.05% yield) was obtained as a red solid.

LC-MS m/z: 390.2 (M+1)⁺.

### Step 3: tert-butyl N-[(3R)-1-(5-amino-1-isopropyl-indazol-4-yl)pyrrolidin-3-yl]carbamate

To a solution of tert-butyl N-[(3R)-1-(1-isopropyl-5-nitro-indazol-4-yl)pyrrolidin-3-yl]carbamate (395 mg, 1.01 mmol, 1 eq) in MeOH (10 mL) was added Pd/C (119.58 mg, 101.43 umol, 10%, w %, 0.1 eq). The reaction mixture was stirred at 25° C. with a balloon of H₂ for 1 hr. LCMS showed starting material was consumed and desired mass detected. The mixture was filtered and filtrate was concentrated under reduced pressure. Compound tert-butyl N-[(3R)-1-(5-amino-1-isopropyl-indazol-4-yl)pyrrolidin-3-yl]carbamate (305 mg, Yield: 83.66% yield) was obtained as brown oil.

LC-MS m/z: 360.2 (M+1)⁺.

### Step 4: tert-butyl N-[(3R)-1-[5-[[1-(2,6-difluorophenyl)-6-oxo-pyridazine-3-carbonyl]amino]-1-isopropyl-indazol-4-yl]pyrrolidin-3-yl]carbamate

To a solution of tert-butyl N-[(3R)-1-(5-amino-1-isopropyl-indazol-4-yl)pyrrolidin-3-yl]carbamate (160 mg, 445.11 umol, 1 eq) in DMF (5.5 mL) was added 1-(2,6-difluorophenyl)-6-oxo-pyridazine-3-carboxylic acid (112.24 mg, 445.11 umol, 1 eq), TEA (90.08 mg, 890.21 umol, 123.91 uL, 2 eq) and HATU (253.86 mg, 667.66 umol, 1.5 eq). The mixture was stirred at 25° C. for 1 hr. LCMS showed the desired mass formed. TLC showed starting material consumed and a new spot formed. The reaction mixture was poured into H₂O (30 mL), extracted with EtOAc (10×3 mL). The combined organic phase dried over anhydrous Na₂SO₄, filtered and concentrated in vacuum. The residue was purified by flash silica gel chromatography (ISCO®; 12 g SepaFlash® Silica Flash Column, eluent of [0^{˜}30]% pet. ether/ethyl acetate gradient). tert-butyl N-[(3R)-1-[5-[[1-(2,6-difluorophenyl)-6-oxo-pyridazine-3-carbonyl]amino]-1-isopropyl-indazol-4-yl]pyrrolidin-3-yl]carbamate (240 mg, Yield: 90.83%) was obtained as a brown solid.

LC-MS m/z: 594.2 (M+1)⁺.

### Step 5: N-[4-[(3R)-3-aminopyrrolidin-1-yl]-1-isopropyl-indazol-5-yl]-1-(2,6-difluorophenyl)-6-oxo-pyridazine-3-carboxamide

To a solution of tert-butyl N-[(3R)-1-[5-[[1-(2,6-difluorophenyl)-6-oxo-pyridazine-3-carbonyl]amino]-1-isopropyl-indazol-4-yl]pyrrolidin-3-yl]carbamate (240 mg, 404.30 umol, 1 eq) in DCM (3 mL) was added TFA (3 mL). The mixture was stirred at 25° C. for 1 hr. LCMS showed the desired mass formed and starting material was consumed. The reaction mixture was concentrated in vacuum to give crude product. The crude product was purified by pre-HPLC (Boston Prime C18 150*30 mm*5 um, water (FA)-ACN, Begin B 15, End B 35, Gradient Time (min) 12, 100% B Hold Time (min) 2, FlowRate (ml/min) 25) Example 179 (116.33 mg, Yield: 58.30%, FA) was obtained as yellow solid.

LC-MS m/z: 494.3 (M+1)⁺.

¹H NMR (400 MHz, CD₃OD) δ 8.24-8.19 (m, 2H), 7.77 (d, J=8.9 Hz, 1H), 7.71-7.62 (m, 1H), 7.35-7.25 (m, 4H), 4.96-4.92 (m, 1H), 3.89-3.83 (m, 1H), 3.82-3.75 (m, 1H), 3.75-3.69 (m, 1H), 3.49 (br dd, J=4.2, 10.3 Hz, 2H), 2.30 (qd, J=7.2, 14.0 Hz, 1H), 2.01-1.91 (m, 1H), 1.53 (d, J=6.7 Hz, 6H).

| No. | Structure | Salt form | MS and 1HNMR |
|---|---|---|---|
| Example 180 | 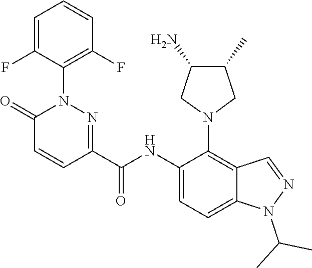 [SMILES: CC(C)n1ncc2c(N3C[C@@H](C)[C@@H](N)C3)c(NC(=O)c3ccc(=O)n(-c4c(F)cccc4F)n3)ccc21] | HCOOH Salt | LC-MS m/z: 508.6 (M + 1)⁺. ¹H NMR (400 MHz, CD₃OD) δ 8.27- 8.19 (m, 2H), 7.67 (tt, J = 8.7, 2.9 Hz, 2H), 7.31 (dt, J = 8.6, 5.6 Hz, 4H), 7.17 (d, J = 8.9 Hz, 1H), 3.99 (dd, J = 10.2, 5.9 Hz, 1H), 3.72- 3.59 (m, 2H), 3.54-3.41 (m, 2H), 2.49 (p, J = 7.2 Hz, 1H), 1.54 (dd, J = 6.7, 1.5 Hz, 7H), 1.09 (d, J = 7.1 Hz, 3H), 1.05 (d, J = 7.2 Hz, 1H). |
| | N-(4-((3R,4R)-3-amino-4- | | |
| | methylpyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 181 | 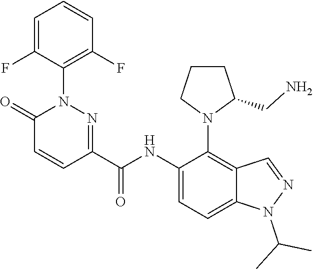 [SMILES: CC(C)n1ncc2c(N3CCC[C@@H]3CN)c(NC(=O)c3ccc(=O)n(-c4c(F)cccc4F)n3)ccc21] | HCOOH Salt | LC-MS m/z: 508.3 (M + 1)⁺. ¹H NMR (400 MHz, CD₃OD) δ 8.26- 8.13 (m, 3H), 7.71 (tt, J = 8.6, 6.2 Hz, 1H), 7.46 (dd, J = 9.1, 0.9 Hz, 1H), 7.41-7.28 (m, 3H), 4.99 (q, J = 6.7 Hz, 1H), 4.20-4.08 (m, 1H), 3.37 (dd, J = 5.7, 3.4 Hz, 1H), 3.17 (dt, J = 9.2, 7.2 Hz, 1H), 2.85-2.59 (m, 2H), 2.34 (dtd, J = 12.4, 7.6, 6.2 Hz, 1H), 2.08-1.93 (m, 1H), 1.87- 1.62 (m, 2H), 1.56 (dd, J = 6.6, 3.1 Hz, 6H). |
| | (R)-N-(4-(2- | | |
| | (aminomethyl)pyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 182 | 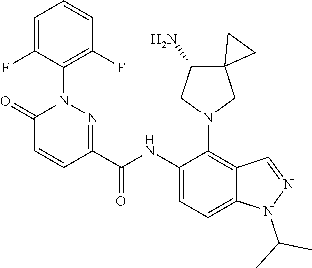 [SMILES: CC(C)n1ncc2c(N3C[C@H](N)C4(CC4)C3)c(NC(=O)c3ccc(=O)n(-c4c(F)cccc4F)n3)ccc21] | HCOOH Salt | LC-MS m/z: 520.3 (M + 1)⁺ ¹H NMR (400 MHz, CD₃OD) δ 8.30- 8.15 (m, 2H), 7.75 (d, J = 8.9 Hz, 1H), 7.66 (ddd, J = 14.8, 8.6, 6.2 Hz, 1H), 7.38-7.19 (m, 4H), 4.97- 4.91 (m, 1H), 4.16 (dd, J = 10.3, 5.6 Hz, 1H), 3.97 (d, J = 9.2 Hz, 1H), 3.61 (dd, J = 10.3, 2.9 Hz, 1H), 3.28 (m, 1Hz), 3.11 (d, J = 9.2 Hz, 1H), 1.53 (dd, J = 6.8, 2.8 Hz, 6H), 0.95 (dt, J = 9.8, 5.2 Hz, 1H), 0.68 (dddd, J = 26.4, 10.2, 7.9, 5.4 Hz, 3H). |
| | (R)-N-(4-(7-amino-5- | | |
| | azaspiro[2.4]heptan-5-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 183 | 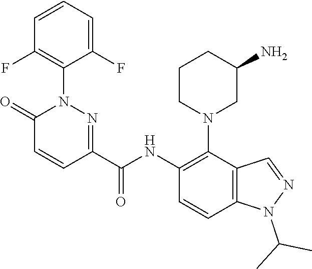 [SMILES: CC(C)n1ncc2c(N3CCC[C@@H](N)C3)c(NC(=O)c3ccc(=O)n(-c4c(F)cccc4F)n3)ccc21] | HCl Salt | LC-MS m/z: 508.3(M + 1)⁺. ¹H NMR (400 MHz, CD₃OD) δ 8.55- 8.43 (m, 1H), 8.27-8.17 (m, 2H), 7.78-7.68 (m, 1H), 7.55-7.48 (m, 1H), 7.44-7.28 (m, 3H), 4.98- 4.94 (m, 1H), 3.38-3.32 (m, 1H), 3.29-3.24 (m, 1H), 3.18-3.04 (m, 2H), 3.03-2.94 (m, 1H), 1.96- 1.83 (m, 1H), 1.80-1.69 (m, 1H), 1.61-1.49 (m, 7H), 1.44-1.29 (m, 1H). |
| | (R)-N-(4-(3-aminopiperidin-1-yl)- | | |
| | 1-isopropyl-1H-indazol-5-yl)-1- | | |
| | (2,6-difluorophenyl)-6-oxo-1,6- | | |
| | dihydropyridazine-3- | | |
| | carboxamide | | |
| Example 184 | 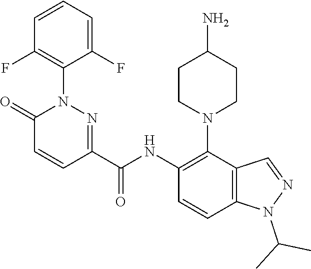 | HCl Salt | LC-MS m/z: 5083 (M + 1)⁺. H NMR (400 MHz, CD₃OD δ 8.27- 8.24 (m, 2 H), 8.13 (d 1 H), 7.71- 7.68 (m, 1 H), 7.46 (d, 1 H), 7.36- 7.32 (m, 3 H), 5.0-4.9 (m, 1 H), 3.43-3.40 (m, 2 H), 1.73-1.70 (m, 2 H), 1.55 (6 H). |
| | N-(4-(4-aminopiperidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 185 | 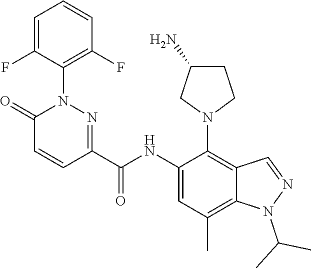 [SMILES: Cc1cc(NC(=O)c2ccc(=O)n(-c3c(F)cccc3F)n2)c(N2CC[C@@H](N)C2)c2cnn(C(C)C)c12] | HCl Salt | LC-MS m/z: 508.3 (m + 1)⁺. ¹H NMR (500 MHz, CD₃OD) δ 8.27- 8.24 (m, 1H), 8.22-8.18 (m, 1H), 7.73-7.63 (m, 2H), 7.35-7.27 (m, 3H), 5.36-5.28 (m, 1H), 3.87- 3.63 (m, 3H), 3.57-3.36 (m, 2H), 2.76-2.68 (m, 3H), 2.40-2.25 (m, 1H), 209-1.95 (m, 1H), 1.57- 1.52 (m, 6H). |
| | (R)-N-(4-(3-aminopyrrolidin-1- | | |
| | yl)-1-isopropyl-7-methyl-1H- | | |
| | indazol-5-yl)-1-(2,6- | | |
| | difluorophenyl)-6-oxo-1,6- | | |
| | dihydropyridazine-3- | | |
| | carboxamide | | |
| Example 186 | 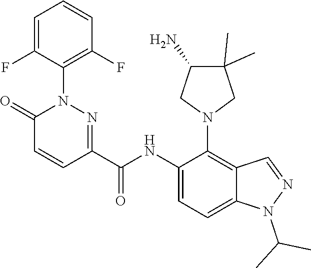 [SMILES: CC(C)n1ncc2c(N3C[C@H](N)C(C)(C)C3)c(NC(=O)c3ccc(=O)n(-c4c(F)cccc4F)n3)ccc21] | HCl Salt | LC-MS m/z: 522.3 (M + 1)⁺. ¹H NMR (500 MHz, CD₃OD) δ 8.29- 8.19 (m, 2H), 7.77 (d, J = 9.0 Hz, 1H), 7.71-7.62 (m, 1H), 7.36- 7.21 (m, 4H), 4.97-4.94 (m, 1H), 4.09 (dd, J = 9.3 Hz, 1H), 3.51-3.46 (m, 2H), 3.41-3.37 (m, 1H), 1.55 (d, J = 6.7 Hz), 1.16 (d, J = 11.7 Hz, 6H). |
| | (R)-N-(4-(4-amino-3,3- | | |
| | dimethylpyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 187 | 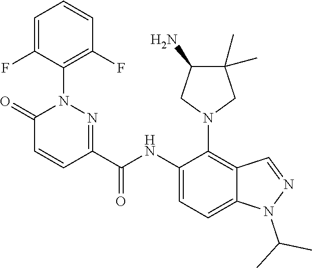 [SMILES: CC(C)n1ncc2c(N3C[C@@H](N)C(C)(C)C3)c(NC(=O)c3ccc(=O)n(-c4c(F)cccc4F)n3)ccc21] | HCl Salt | LC-MS m/z: 522.3 (M + 1)⁺. ¹H NMR (500 MHz, CD₃OD) δ 8.31- 8.20 (m, 2H), 7.78 (d, J = 9.0 Hz, 1H), 7.72-7.64 (m, 1H), 7.38-7.19 (m, 4H), 4.98-4.93 (m, 1H), 4.09 (dd, J = 6.3, 9.8 Hz, 1H), 3.58 (d, J = 9.2 Hz, 1H), 3.53-3.45 (m, 2H), 3.38 (d, J = 9.3 Hz, 1H), 1.55 (d, J = 6.6 Hz, 6H), 1.16 (d, J = 12.4 Hz, 6H). |
| | (S)-N- | | |
| | (4-(4-amino-3,3- | | |
| | dimethylpyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 188 | 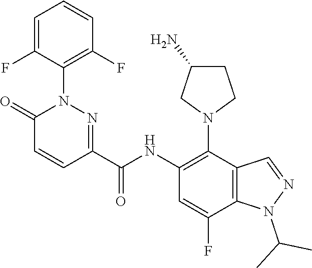 [SMILES: CC(C)n1ncc2c(N3CC[C@@H](N)C3)c(NC(=O)c3ccc(=O)n(-c4c(F)cccc4F)n3)cc(F)c21] | HCl Salt | LC-MS m/z: 512.2 (M + 1)⁺. ¹H NMR (500 MHz, CD₃OD) δ 8.32- 8.20 (m, 2H), 8.09 (d, J = 13.6 Hz, 1H), 7.77-7.64 (m, 1H), 7.43- 7.27 (m, 3H), 5.20-5.12 (m, 1H), 3.66-3.60 (m, 2H), 3.56-3.49 (m, 1H), 3.29-3.22 (m, 2H), 2.27- 2.17 (m, 1H), 1.88-1.79 (m, 1H), 1.58 (d, J = 6.6 Hz, 5H). |
| | (R)-N-(4-(3-aminopyrrolidin-1- | | |
| | yl)-7-fluoro-1-isopropyl-1H- | | |
| | indazol-5-yl)-1-(2,6- | | |
| | difluorophenyl)-6-oxo-1,6- | | |
| | dihydropyridazine-3- | | |
| | carboxamide | | |
| Example 189 | 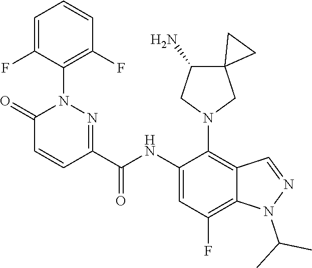 [SMILES: CC(C)n1ncc2c(N3C[C@H](N)C4(CC4)C3)c(NC(=O)c3ccc(=O)n(-c4c(F)cccc4F)n3)cc(F)c21] | HCOOH Salt | LC-MS m/z: 538.2 (M + 1)+ 1H NMR (400 MHz, CD₃OD) δ 8.52 (s, 1H), 8.31 (s, 1H), 8.24 (d, J = 9.9 Hz, 1H), 8.04 (d, J = 13.6 Hz, 1H), 7.68 (tt, J = 8.5, 6.2 Hz, 1H), 7.32 (t, J = 9.6 Hz, 3H), 5.15 (h, J = 6.7 Hz, 1H), 3.95 (dd, J = 10.2, 6.4 Hz, 1H), 3.78 (d, J = 9.0 Hz, 1H), 3.45 (dd, J = 10.4, 4.3 Hz, 1H), 3.29 (d, J = 5.0 Hz, 1H), 2.92 (d, J = 9.0 Hz, 1H), 1.56 (dd, J = 6.5, 2.2 Hz, 6H), 0.92 (dt, J = 10.4, 5.5 Hz, 1H), 0.75-0.58 (m, 2H), 0.47-0.37 (m, 1H). |
| | (R)-N-(4-(7-amino-5- | | |
| | azaspiro[2.4]heptan-5-yl)-7- | | |
| | fluoro-1-isopropyl-1H-indazol-5- | | |
| | yl)-1-(2,6-difluorophenyl)-6-oxo- | | |
| | 1,6-dihydropyridazine-3- | | |
| | carboxamide | | |
| Example 190 | 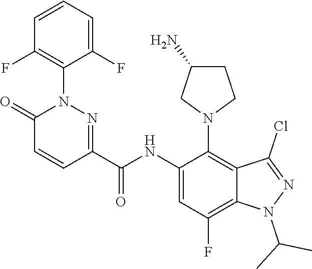 [SMILES: CC(C)n1nc(Cl)c2c(N3CC[C@@H](N)C3)c(NC(=O)c3ccc(=O)n(-c4c(F)cccc4F)n3)cc(F)c21] | HCl Salt | LC-MS m/z: 546.2 (M + 1)⁺. ¹H NMR (400 MHz, CD₃OD) δ 8.30 (d, J = 13.8 Hz, 1H), 8.24 (d, J = 9.8 Hz, 1H), 7.87-7.61 (m, 1H), 7.36 (dd, J = 16.0, 9.6 Hz, 3H), 5.12 (dt, J = 13.2, 6.6 Hz, 1H), 3.72-3.51 (m, 3H), 3.40 (dt, J = 12.2, 5.5 Hz, 2H), 2.29 (dt, J = 12.8, 6.4 Hz, 1H), 2.07- 1.89 (m, 1H), 1.56 (d, J = 6.6 Hz, 6H). |
| | (R)-N-(4-(3-aminopyrrolidin-1- | | |
| | yl)-3-chloro-7-fluoro-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 191 | 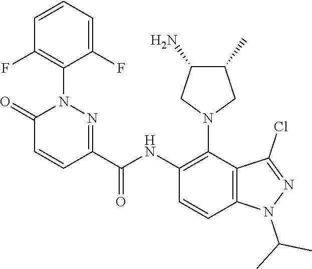 [SMILES: CC(C)n1nc(Cl)c2c(N3C[C@@H](C)[C@@H](N)C3)c(NC(=O)c3ccc(=O)n(-c4c(F)cccc4F)n3)ccc21] | HCl Salt | LC-MS m/z: 542.1 (M + 1)⁺. ¹H NMR (400 MHz, CD₃OD) δ 8.32 (d, J = 9.5 Hz, 1H), 8.26 (d, J = 9.9 Hz, 1H), 7.75-7.67 (m, 1H), 7.60 (d, J = 9.3 Hz, 1H), 7.34 (dd, J = 9.2, 6.1 Hz, 3H), 5.06-4.96 (m, 1H), 4.63 (s, 1H), 3.91 (s, 1H), 3.81- 3.71 (m, 1H), 3.47 (d, J = 26.8 Hz, 1H), 3.15 (t, J = 7.4 Hz, 1H), 2.60- 2.45 (m, 1H), 1.54 (d, J = 6.2 Hz, 6H), 1.31 (s, 1H), 1.01 (d, J = 7.2 Hz, 3H). |
| | N-(4-((3R,4R)-3-amino-4- | | |
| | methylpyrrolidin-1-yl)-3-chloro- | | |
| | 1-isopropyl-1H-indazol-5-yl)-1- | | |
| | (2,6-difluorophenyl)-6-oxo-1,6- | | |
| | dihydropyridazine-3- | | |
| | carboxamide | | |
| Example 192 | 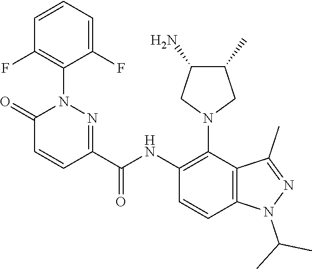 [SMILES: Cc1nn(C(C)C)c2ccc(NC(=O)c3ccc(=O)n(-c4c(F)cccc4F)n3)c(N3C[C@@H](C)[C@@H](N)C3)c12] | HCl Salt | LC-MS m/z: 522.6 (M + 1)⁺. ¹H NMR (400 MHz, CD₃OD) δ 8.23 (d, J = 9.9 Hz, 1H), 7.68 (dd, J = 14.0, 7.6 Hz, 2H), 7.45 (d, J = 9.0 Hz, 1H), 7.32 (dd, J = 8.9, 6.2 Hz, 3H), 3.84 (dd, J = 11.6, 6.5 Hz, 1H), 3.72 (dd, J = 10.4, 3.7 Hz, 1H), 3.62 (dd, J = 6.1, 3.5 Hz, 1H), 3.20-3.03 (m, 1H), 2.75-2.41 (m, 4H), 1.49 (t, J = 26.0 Hz, 6H), 1.08 (d, J = 7.1 Hz, 3H). |
| | N-(4-((3R,4R)-3-amino-4- | | |
| | methylpyrrolidin-1-yl)-1- | | |
| | isopropyl-3-methyl-1H-indazol- | | |
| | 5-yl)-1-(2,6-difluorophenyl)-6- | | |
| | oxo-1,6-dihydropyridazine-3- | | |
| | carboxamide | | |
| Example 193 | 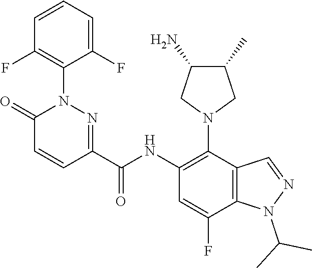 [SMILES: CC(C)n1ncc2c(N3C[C@@H](C)[C@@H](N)C3)c(NC(=O)c3ccc(=O)n(-c4c(F)cccc4F)n3)cc(F)c21] | HCl Salt | LC-MS m/z: 526.5 (M + 1)⁺. ¹H NMR (400 MHz, CD₃OD) δ 8.26 (dd, J = 8.2, 5.9 Hz, 2H), 8.08 (d, J = 13.7 Hz, 1H), 7.79-7.63 (m, 1H), 7.33 (t, J = 8.3 Hz, 3H), 5.14 (dd, J = 13.3, 6.7 Hz, 1H), 3.66 (dd, J = 9.5, 6.4 Hz, 1H), 3.47 (dd, J = 11.2, 6.3 Hz, 1H), 3.15-2.96 (m, 2H), 2.30 (dt, J = 13.8, 6.9 Hz, 1H), 1.57 (d, J = 6.6 Hz, 6H), 0.96 (d, J = 7.1 Hz, 3H). |
| | N-(4-((3R,4R)-3-amino-4- | | |
| | methylpyrrolidin-1-yl)-7-fluoro- | | |
| | 1-isopropyl-1H-indazol-5-yl)-1- | | |
| | (2,6-difluorophenyl)-6-oxo-1,6- | | |
| | dihydropyridazine-3- | | |
| | carboxamide | | |
| Example 194 | 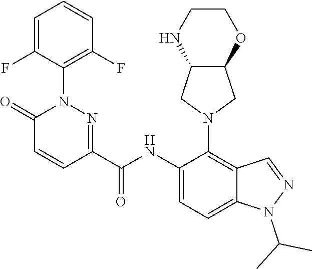 [SMILES: CC(C)n1ncc2c(N3C[C@@H]4NCCO[C@H]4C3)c(NC(=O)c3ccc(=O)n(-c4c(F)cccc4F)n3)ccc21] | HCl Salt | LC-MS m/z: 536.5 (M + 1)⁺. 1H NMR (400 MHz, CD₃OD) δ 8.22- 8.16 (m, 2H), 7.72-7.61 (m, 1H), 7.49-7.44 (m, 1H), 7.33-7.25 (m, 3H), 7.21-7.15 (m, 1H), 4.95-4.90 (m, 1H), 4.28-4.20 (m, 1H), 4.12- 4.03 (m, 1H), 4.01-3.91 (m, 1H), 3.85-3.79 (m, 2H), 3.75-3.65 (m, 2H), 3.53-3.45 (m, 2H), 3.38-3.33 (m, 1H), 1.55-1.51 (m, 6H) |
| | 1-(2,6-difluorophenyl)-N-(4- | | |
| | ((4aS,7aS)- | | |
| | hexahydropyrrolo[3,4- | | |
| | b][1,4]oxazin-6(2H)-yl)-1- | | |
| | isopropyl-1H-indazol-5-yl)-6- | | |
| | oxo-1,6-dihydropyridazine-3- | | |
| | carboxamide | | |
| Example 195 | 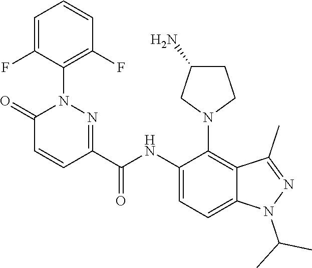 [SMILES: Cc1nn(C(C)C)c2ccc(NC(=O)c3ccc(=O)n(-c4c(F)cccc4F)n3)c(N3CC[C@@H](N)C3)c12] | HCl Salt | LC-MS m/z: 508.3 (M + 1)⁺. 1H NMR (400 MHz, CD₃OD) δ 8.23- 8.18 (m, 1H), 7.76-7.63 (m, 2H), 7.47-7.41 (m, 1H), 7.35-7.27 (m, 3H), 4.92-4.90 (m, 1H), 3.82- 3.65 (m, 2H), 3.57-3.47 (m, 1H), 3.45-3.36 (m, 1H), 3.30-3.27 (m, 1H), 2.71-2.66 (m, 3H), 2.45- 2.31 (m, 1H), 2.05-1.92 (m, 1H), 1.54-1.49 (m, 6H) |
| | (R)-N-(4-(3-aminopyrrolidin-1- | | |
| | yl)-1-isopropyl-3-methyl-1H- | | |
| | indazol-5-yl)-1-(2,6- | | |
| | difluorophenyl)-6-oxo-1,6- | | |
| | dihydropyridazine-3- | | |
| | carboxamide | | |
| Example 196 (isomer 1) | 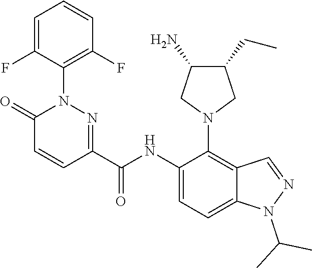 [SMILES: CC[C@@H]1CN(c2c(NC(=O)c3ccc(=O)n(-c4c(F)cccc4F)n3)ccc3c2cnn3C(C)C)C[C@@H]1N] | HCl Salt | LC-MS m/z: 522.1 (M + 1)⁺. ¹H NMR (400 MHz, CD₃OD) δ 8.15- 8.07 (m, 2H), 7.61-7.51 (m, 1H), 7.36 (d, J = 9.0 Hz, 1H), 7.23-7.14 (m, 3H), 7.05 (d, J = 8.5 Hz, 1H), 4.88-4.81 (m, 1H), 4.05 (dd, J = 5.3, 10.8 Hz, 1H), 3.77 (br s, 1H), 3.61- 3.50 (m, 2H), 3.48-3.40 (m, 1H), 2.37-2.24 (m, 1H), 2.37-2.24 (m, 1H), 1.54-1.46 (m, 1H), 1.42 (dd, J = 2.5, 6.8 Hz, 6H), 1.39-1.29 (m, 1H), 0.89 (t, J = 7.4 Hz, 3H) |
| | N-(4- | | |
| | ((3R,4R)-3-amino-4- | | |
| | ethylpyrrolidin-1-yl)-1-isopropyl- | | |
| | 1H-indazol-5-yl)-1-(2,6- | | |
| | difluorophenyl)-6-oxo-1,6- | | |
| | dihydropyridazine-3- | | |
| | carboxamide | | |
| Example 197 (isomer 2) | 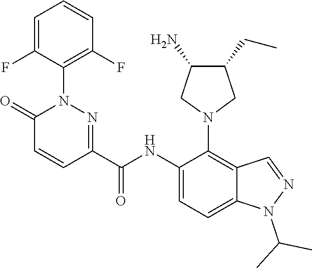 [SMILES: CC[C@@H]1CN(c2c(NC(=O)c3ccc(=O)n(-c4c(F)cccc4F)n3)ccc3c2cnn3C(C)C)C[C@@H]1N] | HCl Salt | LC-MS m/z: 522.1 (M + 1)⁺. ¹H NMR (400 MHz, CD₃OD) δ 8.30- 8.17 (m, 2H), 7.74-7.63 (m, 1H), 7.48 (d, J = 8.8 Hz, 1H), 7.37-7.27 (m, 3H), 7.17 (d, J = 8.8 Hz, 1H), 4.98-4.94 (m, 1H), 4.17 (dd, J = 5.4, 10.7 Hz, 1H), 3.89 (br s, 1H), 3.74- 3.61 (m, 2H), 3.61-3.52 (m, 1H), 2.43 (br d, J = 8.0 Hz, 1H), 1.66- 1.57 (m, 1H), 1.54 (dd, J = 2.5, 6.5 Hz, 6H), 1.50-1.41 (m, 1H), 1.01 (t, J = 7.4 Hz, 3H) |
| | N-(4- | | |
| | ((35,4S)-3-amino-4- | | |
| | ethylpyrrolidin-1-yl)-1-isopropyl- | | |
| | 1H-indazol-5-yl)-1-(2,6- | | |
| | difluorophenyl)-6-oxo-1,6- | | |
| | dihydropyridazine-3- | | |
| | carboxamide | | |
| Example 198 | 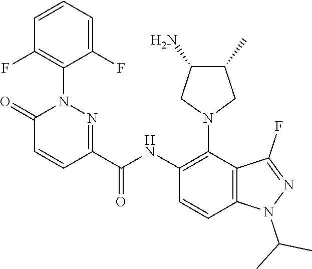 [SMILES: CC(C)n1nc(F)c2c(N3C[C@@H](C)[C@@H](N)C3)c(NC(=O)c3ccc(=O)n(-c4c(F)cccc4F)n3)ccc21] | HCl Salt | LC-MS m/z: 526.2 (M + 1)⁺. ¹H NMR (400 MHz, CD₃OD) δ 8.26 (dd, J = 9.5, 6.4 Hz, 2H), 7.76-7.62 (m, 1H), 7.45 (d, J = 10.7 Hz, 1H), 7.38-7.25 (m, 3H), 3.83 (dd, J = 13.5, 7.0 Hz, 1H), 3.74-3.64 (m, 1H), 3.50-3.42 (m, 1H), 3.40- 3.36 (m, 1H), 3.19 (d, J = 30.5 Hz, 1H), 3.10-2.98 (m, 2H), 2.88 (s, 1H), 2.54 (dt, J = 13.9, 6.9 Hz, 1H), 1.49 (d, J = 6.6 Hz, 6H), 1.45 (d, J = 6.7 Hz, 1H), 1.05 (d, J = 7.2 Hz, 3H). |
| | N-(4-((3R,4R)-3-amino-4- | | |
| | methylpyrrolidin-1-yl)-3-fluoro- | | |
| | 1-isopropyl-1H-indazol-5-yl)-1- | | |
| | (2,6-difluorophenyl)-6-oxo-1,6- | | |
| | dihydropyridazine-3- | | |
| | carboxamide | | |
| Example 199 | 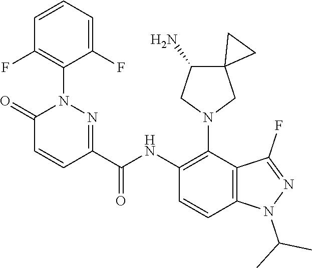 [SMILES: CC(C)n1nc(F)c2c(N3C[C@H](N)C4(CC4)C3)c(NC(=O)c3ccc(=O)n(-c4c(F)cccc4F)n3)ccc21] | HCl Salt | LC-MS m/z: 538.2 (M + 1)⁺. ¹H NMR (400 MHz, CD₃OD) δ 8.25 (d, J = 9.9 Hz, 1H), 8.13 (d, J = 9.2 Hz, 1H), 7.76-7.61 (m, 1H), 7.44 (d, J = 7.7 Hz, 1H), 7.32 (dd, J = 9.1, 4.8 Hz, 3H), 3.96-3.87 (m, 1H), 3.61 (d, J = 9.1 Hz, 1H), 3.51 (d, J = 7.5 Hz, 1H), 3.37 (s, 1H), 3.02 (d, J = 8.1 Hz, 1H), 1.51 (t, J = 9.1 Hz, 6H), 0.95 (dd, J = 10.4, 5.2 Hz, 1H), 0.77 (dd, J = 13.7, 7.6 Hz, 1H), 0.67 (d, J = 5.0 Hz, 1H), 0.54 (dd, J = 9.7, 5.4 Hz, 1H). |
| | (R)-N-(4-(7-amino-5- | | |
| | azaspiro[2.4]heptan-5-yl)-3- | | |
| | fluoro-1-isopropyl-1H-indazol-5- | | |
| | yl)-1-(2,6-difluorophenyl)-6-oxo- | | |
| | 1,6-dihydropyridazine-3- | | |
| | carboxamide | | |
| Example 200 | 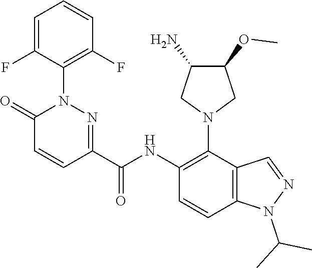 [SMILES: CO[C@H]1CN(c2c(NC(=O)c3ccc(=O)n(-c4c(F)cccc4F)n3)ccc3c2cnn3C(C)C)C[C@@H]1N] | HCl Salt | LC-MS m/z: 524.2 (M + 1)⁺. ¹H NMR (500 MHz, CD₃OD) δ 8.31- 8.16 (m, 2H), 7.80 (d, J = 9.0 Hz, 1H), 7.72-7.58 (m, 1H), 7.41-7.21 (m, 4H), 4.96-4.92 (m, 1H), 4.02- 3.98 (m, 1H), 3.91 (dd, J = 6.6, 9.9 Hz, 1H), 3.88-3.83 (m, 1H), 3.66- 3.62 (m, 1H), 3.53-3.45 (m, 2H), 3.30 (br s, 3H), 1.54 (d, J = 6.6 Hz, 6H). |
| | N-(4- | | |
| | ((3R,4R)-3-amino-4- | | |
| | methoxypyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 201 | 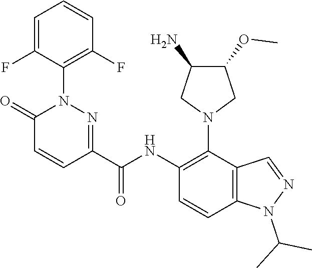 [SMILES: CO[C@@H]1CN(c2c(NC(=O)c3ccc(=O)n(-c4c(F)cccc4F)n3)ccc3c2cnn3C(C)C)C[C@H]1N] | HCl Salt | LC-MS m/z: 524.2 (M + 1)⁺. ¹H NMR (500 MHz, CD₃OD) δ 8.25- 8.19 (m, 2H), 7.79 (d, J = 9.0 Hz, 1H), 7.70-7.62 (m, 1H), 7.34-7.26 (m, 4H), 4.97-4.93 (m, 1H), 3.99 (td, J = 3.2, 6.0 Hz, 1H), 3.92 (dd, J = 6.6, 10.1 Hz, 1H), 3.86 (dd, J = 5.9, 10.6 Hz, 1H), 3.66-3.61 (m, 1H), 3.53-3.46 (m, 2H), 3.30 (br s, 3H), 1.54 (d, J = 6.7 Hz, 6H). |
| | N-(4- | | |
| | ((35,4S)-3-amino-4- | | |
| | methoxypyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 202 | 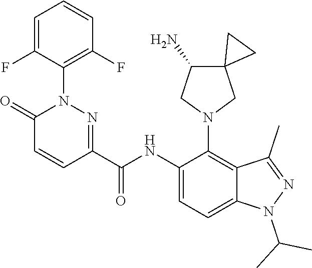 [SMILES: Cc1nn(C(C)C)c2ccc(NC(=O)c3ccc(=O)n(-c4c(F)cccc4F)n3)c(N3C[C@H](N)C4(CC4)C3)c12] | HCl Salt | LC-MS m/z: 534.2 (M + 1)⁺. ¹H NMR (400 MHz, CD₃OD) δ 8.24- 8.19 (m, 1H), 7.71-7.62 (m, 1H), 7.53-7.47 (m, 1H), 7.45-7.40 (m, 1H), 7.35-7.26 (m, 3H), 4.87-4.81 (m, 1H), 4.03-3.95 (m, 1H), 3.67- 3.62 (m, 1H), 3.47-3.41 (m, 2H), 3.21-3.17 (m, 1H), 2.73-2.71 (m, 3H), 1.53-1.50 (m, 6H), 1.02-0.92 (m, 1H), 0.82-0.75 (m, 1H), 0.74- 0.66 (m, 2H). |
| | (R)-N-(4-(7-amino-5- | | |
| | azaspiro[2.4]heptan-5-yl)-1- | | |
| | isopropyl-3-methyl-1H-indazol- | | |
| | 5-yl)-1-(2,6-difluorophenyl)-6- | | |
| | oxo-1,6-dihydropyridazine-3- | | |
| | carboxamide | | |
| Example 203 | 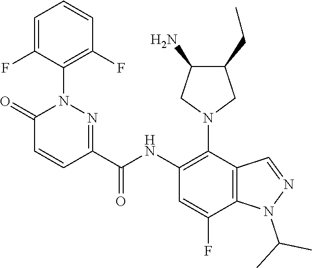 [SMILES: CC[C@H]1CN(c2c(NC(=O)c3ccc(=O)n(-c4c(F)cccc4F)n3)cc(F)c3c2cnn3C(C)C)C[C@H]1N] | HCl Salt | LC-MS m/z: 540.2 (M + 1)⁺. ¹H NMR (400 MHz, CD₃OD) δ 8.31- 8.17 (m, 2H), 7.83-7.64 (m, 2H), 7.38-7.25 (m, 3H), 5.14 (td, J = 6.4, 13.2 Hz, 1H), 3.93 (dd, J = 6.2, 10.6 Hz, 1H), 3.81 (br d, J = 3.3 Hz, 1H), 3.52-3.46 (m, 1H), 3.42-3.36 (m, 2H), 2.30 (br s, 1H), 1.66-1.60 (m, 1H), 1.58-1.54 (m, 6H), 1.49-1.38 (m, 1H), 0.95 (t, J = 7.4 Hz, 3H). |
| | N-(4- | | |
| | ((35,4S)-3-amino-4- | | |
| | ethylpyrrolidin-1-yl)-7-fluoro-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 204 | 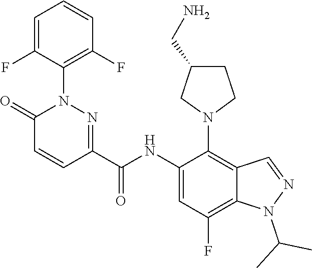 [SMILES: CC(C)n1ncc2c(N3CC[C@@H](CN)C3)c(NC(=O)c3ccc(=O)n(-c4c(F)cccc4F)n3)cc(F)c21] | HCl Salt | LC-MS m/z: 526.5 (M + 1)⁺. 1H NMR (400 MHz, CD₃OD) δ 8.31- 8.19 (m, 2H), 8.08 (s, 1H), 7.79- 7.62 (m, 1H), 7.42-7.31 (m, 3H), 5.16 (dd, J = 13.1, 6.7 Hz, 1H), 4.34 (d, J = 7.2 Hz, 1H), 3.62 (s, 4H), 3.39 (s, 2H), 2.89 (s, 2H), 2.64 (s, 1H), 2.20 (s, 1H), 1.58 (d, J = 6.6 Hz, 7H). |
| | (S)-N-(4-(3- | | |
| | (aminomethyl)pyrrolidin-1-yl)-7- | | |
| | fluoro-1-isopropyl-1H-indazol-5- | | |
| | yl)-1-(2,6-difluorophenyl)-6-oxo- | | |
| | 1,6-dihydropyridazine-3- | | |
| | carboxamide | | |
| Example 205 | 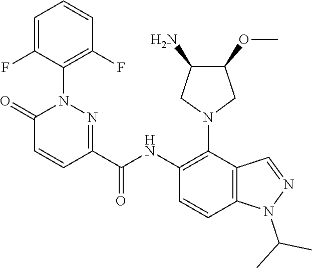 [SMILES: CO[C@H]1CN(c2c(NC(=O)c3ccc(=O)n(-c4c(F)cccc4F)n3)ccc3c2cnn3C(C)C)C[C@H]1N] | Free base | LC-MS m/z: 524.2(M + 1)⁺. ¹H NMR (400 MHz, CD₃OD) δ 8.26- 8.19 (m, 2H), 7.75 (d, J = 8.9 Hz, 1H), 7.73-7.63 (m, 1H), 7.36-7.22 (m, 4H), 4.98-4.94 (m, 1H), 4.05- 3.99 (m, 1H), 3.82-3.58 (m, 5H), 3.41 (s, 3H), 1.59-1.52 (m, 6H). |
| | N-(4- | | |
| | ((3R,4S)-3-amino-4- | | |
| | methoxypyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 206 | 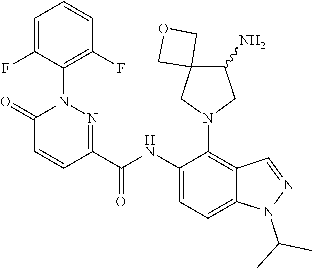 [SMILES: CC(C)n1ncc2c(N3CC(N)C4(COC4)C3)c(NC(=O)c3ccc(=O)n(-c4c(F)cccc4F)n3)ccc21] | HCOOH Salt | LC-MS m/z: 536.2 (M + 1)⁺. ¹H NMR (400 MHz, CD₃OD) δ 8.19- 8.08 (m, 2H), 7.68 (d, J = 8.9 Hz, 1H), 7.57 (tt, J = 8.6, 6.2 Hz, 1H), 7.18 (dd, J = 18.6, 9.4 Hz, 4H), 4.70 (d, J = 7.0 Hz, 1H), 4.46 (d, J = 7.0 Hz, 1H), 4.39 (d, J = 6.2 Hz, 1H), 4.23 (d, J = 6.2 Hz, 1H), 3.87 (d, J = 9.7 Hz, 1H), 3.79 (dd, J = 10.2, 6.2 Hz, 1H), 3.75-3.69 (m, 2H), 1.43 (d, J = 6.6 Hz, 6H). |
| | N-(4-(8-amino-2-oxa-6- | | |
| | azaspiro[3.4]octan-6-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 207 | 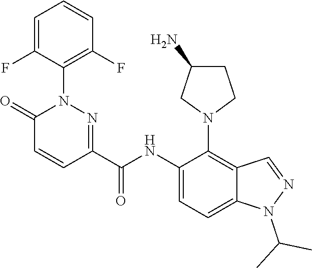 [SMILES: CC(C)n1ncc2c(N3CC[C@H](N)C3)c(NC(=O)c3ccc(=O)n(-c4c(F)cccc4F)n3)ccc21] | HCl Salt | LC-MS m/z: 494.2 (M + 1)⁺. ¹H NMR (400 MHz, CD₃OD) δ 8.24- 8.19 (m, 2H), 7.76-7.67 (m, 2H), 7.32-7.28 (m, 4H), 4.96-4.86 (m, 1H), 3.91-3.79 (m, 3H), 3.60-3.42 (m, 2H), 2.38-2.28 (m, 1H), 2.10- 2.01 (m, 1H), 1.54-1.501 (m, 6H). |
| | (S)-N-(4-(3-aminopyrrolidin-1- | | |
| | yl)-1-isopropyl-1H-indazol-5-yl)- | | |
| | 1-(2,6-difluorophenyl)-6-oxo- | | |
| | 1,6-dihydropyridazine-3- | | |
| | carboxamide | | |
| Example 208 | 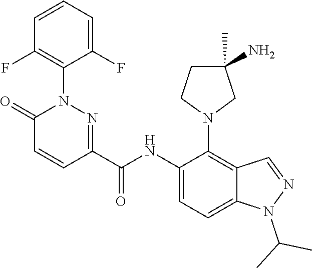 [SMILES: CC(C)n1ncc2c(N3CC[C@@](C)(N)C3)c(NC(=O)c3ccc(=O)n(-c4c(F)cccc4F)n3)ccc21] | HCOOH Salt | LC-MS m/z: 508.2 (M + 1)⁺. ¹H NMR (400 MHz, CD₃OD) δ 8.23 (d, J = 9.9 Hz, 2H), 7.93 (d, J = 9.0 Hz, 1H), 7.67 (tt, J = 8.6, 6.2 Hz, 1H), 7.37-7.22 (m, 4H), 4.95 (q, J = 6.7 Hz, 1H), 3.73-3.61 (m, 2H), 3.55-3.46 (m, 2H), 2.16 (ddd, J = 12.9, 8.2, 4.3 Hz, 1H), 2.00 (dt, J = 13.5, 8.0 Hz, 1H), 1.53 (d, J = 6.6 Hz, 6H), 1.34 (s, 3H). |
| | (R)-N-(4-(3-amino-3- | | |
| | methylpyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 209 | 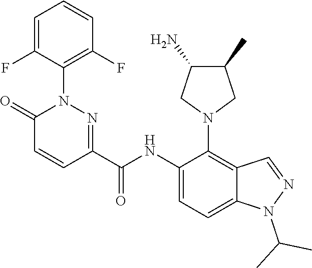 [SMILES: CC(C)n1ncc2c(N3C[C@H](C)[C@@H](N)C3)c(NC(=O)c3ccc(=O)n(-c4c(F)cccc4F)n3)ccc21] | HCOOH Salt | LC-MS m/z: 508.2 (M + 1)⁺. ¹H NMR (400 MHz, CD₃OD) δ 8.53 (s, 1H), 8.22 (d, J = 9.7 Hz, 2H), 7.85 (d, J = 9.0 Hz, 1H), 7.67 (tt, J = 8.5, 6.2 Hz, 1H), 7.36-7.22 (m, 4H), 3.85 (dt, J = 9.3, 7.3 Hz, 2H), 3.45 (dd, J = 9.9, 6.1 Hz, 1H), 3.22 (q, J = 6.1 Hz, 1H), 3.12 (dd, J = 9.4, 5.6 Hz, 1H), 2.22 (p, J = 6.3 Hz, 1H), 1.53 (d, J = 6.6 Hz, 6H), 1.05 (d, J = 6.9 Hz, 3H). |
| | N-(4-((3R,4S)-3-amino-4- | | |
| | methylpyrrolidin-1-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 210 | 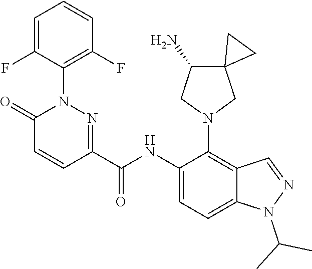 [SMILES: CC(C)n1ncc2c(N3C[C@H](N)C4(CC4)C3)c(NC(=O)c3ccc(=O)n(-c4c(F)cccc4F)n3)ccc21] | HCOOH Salt | LC-MS m/z: 520.3 (M + 1)⁺. ¹H NMR (400 MHz, CD₃OD) δ 8.30- 8.15 (m, 2H), 7.75 (d, J = 8.9 Hz, 1H), 7.66 (ddd, J = 14.8, 8.6, 6.2 Hz, 1H), 7.38-7.19 (m, 4H), 4.97- 4.91 (m, 1H), 4.16 (dd, J =10.3, 5.6 Hz, 1H), 3.97 (d, J = 9.2 Hz, 1H), 3.61 (dd, J = 10.3, 2.9 Hz, 1H), 3.28 (m, 1Hz) 3.11 (d, J = 9.2 Hz, 1H), 1.53 (dd, J = 6.8, 2.8 Hz, 6H), 0.95 (dt, J = 9.8, 5.2 Hz, 1H), 0.68 (dddd, J = 26.4, 10.2, 7.9, 5.4 Hz, 3H). |
| | (R)-N-(4-(7-amino-5- | | |
| | azaspiro[2.4]heptan-5-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 211 | 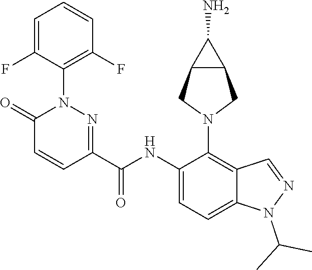 [SMILES: CC(C)n1ncc2c(N3C[C@@H]4[C@@H](N)[C@@H]4C3)c(NC(=O)c3ccc(=O)n(-c4c(F)cccc4F)n3)ccc21] | HCl Salt | LC-MS m/z: 506.2 (M + 1)⁺. ¹H NMR (400 MHz, CD₃OD) δ 8.22- 8.18 (m, 1H), 8.13 (s, 1H), 7.71- 7.62 (m, 1H), 7.60-7.54 (m, 1H), 7.35-7.26 (m, 4H), 4.96-4.90 (m, 1H), 3.71-3.62 (m, 4H), 2.71-2.60 (m, 1H), 2.04-2.01 (m, 2H), 1.53 (d, J = 6.6 Hz, 6H). |
| | N-(4-((1R,5S,6s)-6-amino-3- | | |
| | azabicyclo[3.1.0]hexan-3-yl)-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 212 | 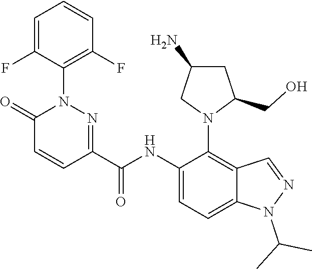 [SMILES: CC(C)n1ncc2c(N3C[C@@H](N)C[C@H]3CO)c(NC(=O)c3ccc(=O)n(-c4c(F)cccc4F)n3)ccc21] | HCl Salt | LC-MS m/z: 524.2 (M + 1)⁺. ¹H NMR (400 MHz, CD₃OD) δ 8.56- 8.49 (m, 1H), 8.31-8.26 (m, 1H), 8.20-8.17 (m, 1H), 7.76-7.67 (m, 1H), 7.64-7.60 (m, 1H), 7.37-7.30 (m, 3H), 5.07-4.98 (m, 1H), 4.02- 3.88 (m, 2H), 3.84-3.76 (m, 1H), 3.30-3.24 (m, 2H), 2.98-2.86 (m, 1H), 2.09-2.00 (m, 1H), 1.60-1.54 (m, 6H). |
| | N-(4-((25,4S)-4-amino-2- | | |
| | (hydroxymethyl)pyrrolidin-1-yl)- | | |
| | 1-isopropyl-1H-indazol-5-yl)-1- | | |
| | (2,6-difluorophenyl)-6-oxo-1,6- | | |
| | dihydropyridazine-3- | | |
| | carboxamide | | |
| Example 213 | 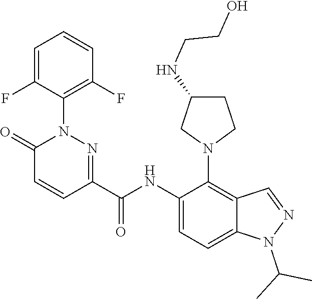 [SMILES: CC(C)n1ncc2c(N3CC[C@@H](NCCO)C3)c(NC(=O)c3ccc(=O)n(-c4c(F)cccc4F)n3)ccc21] | HCl Salt | LC-MS m/z: 538.2 (M + 1)⁺. ¹H NMR (400 MHz, CD₃OD) δ 8.17- 8.08 (m, 2H), 7.76 (d, J = 8.9 Hz, 1H), 7.58 (ddd, J = 14.7, 8.5, 6.2 Hz, 1H), 7.22 (dd, J = 9.5, 6.5 Hz, 4H), 4.91-4.82 (m, 2H), 3.80 (dd, J = 9.8, 6.8 Hz, 1H), 3.70 (t, J = 5.2 Hz, 3H), 3.62-3.48 (m, 2H), 3.41- 3.31 (m, 1H), 3.04 (q, J = 5.5 Hz, 2H), 2.23 (dq, J = 14.2, 7.5 Hz, 1H), 1.96 (tt, J = 13.3, 6.0 Hz, 1H), 1.43 (d, J = 6.6 Hz, 6H). |
| | (R)-1-(2,6-difluorophenyl)-N-(4- | | |
| | (3-((2- | | |
| | hydroxyethyl)amino)pyrrolidin- | | |
| | 1-yl)-1-isopropyl-1H-indazol-5- | | |
| | yl)-6-oxo-1,6-dihydropyridazine- | | |
| | 3-carboxamide | | |
| Example 214 | 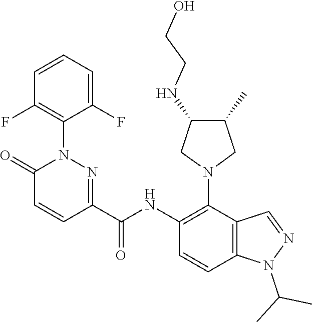 [SMILES: CC(C)n1ncc2c(N3C[C@@H](C)[C@@H](NCCO)C3)c(NC(=O)c3ccc(=O)n(-c4c(F)cccc4F)n3)ccc21] | HCOOH Salt | LC-MS m/z: 552.3 (M + 1)⁺. ¹H NMR (400 MHz, CD₃OD) δ 8.21 (d, J = 10.0 Hz, 2H), 7.75 (d, J = 8.9 Hz, 1H), 7.65 (t, J = 6.2 Hz, 1H), 7.29 (d, J = 9.7 Hz, 3H), 7.15 (d, J = 8.9 Hz, 1H), 4.60 (s, 1H), 3.70 (d, J = 8.5 Hz, 5H), 3.48 (m, 2H), 2.80 (s, 2H), 2.47 (s, 1H), 1.52 (dd, J = 6.6, 1.6 Hz, 6H), 1.00 (d, J = 7.0 Hz, 3H). |
| | 1-(2,6-difluorophenyl)-N-(4- | | |
| | ((3R,4R)-3-((2- | | |
| | hydroxyethyl)amino)-4- | | |
| | methylpyrrolidin-1-yl)-1- | | |
| | isopropyl-1H-indazol-5-yl)-6- | | |
| | oxo-1,6-dihydropyridazine-3- | | |
| | carboxamide | | |
| Example 215 | 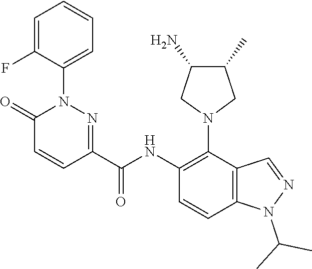 [SMILES: CC(C)n1ncc2c(N3C[C@@H](C)[C@@H](N)C3)c(NC(=O)c3ccc(=O)n(-c4ccccc4F)n3)ccc21] | HCl Salt | LC-MS m/z: 490.3 (M + 1)⁺. ¹H NMR (400 MHz, CD₃OD) δ 8.23- 8.15 (m, 2H), 7.72-7.57 (m, 3H), 7.46-7.35 (m, 2H), 7.25 (dd, J = 15.6, 9.4 Hz, 2H), 4.92 (p, J = 6.6 Hz, 1H), 4.04 (dd, J = 10.6, 6.1 Hz, 1H), 3.77 (d, J = 4.5 Hz, 1H), 3.65 (dd, J = 9.3, 7.5 Hz, 1H), 3.53 (dd, J = 10.6, 3.9 Hz, 1H), 3.43 (t, J = 8.9 Hz, 1H), 2.60-2.50 (m, 1H), 1.53 (dd, J = 6.6, 1.0 Hz, 6H), 1.11 (d, J = 7.1 Hz, 3H). |
| | N-(4-((3R,4R)-3-amino-4- | | |
| | methylpyrrolidin-1-yl)-1- | | |
| | isopropyl-1H-indazol-5-yl)-1-(2- | | |
| | fluorophenyl)-6-oxo-1,6- | | |
| | dihydropyridazine-3- | | |
| | carboxamide | | |
| Example 216 | 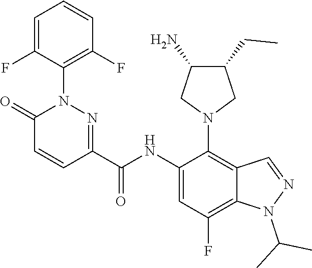 [SMILES: CC[C@@H]1CN(c2c(NC(=O)c3ccc(=O)n(-c4c(F)cccc4F)n3)cc(F)c3c2cnn3C(C)C)C[C@@H]1N] | HCl Salt | LC-MS m/z: 540.3 (M + 1)⁺. ¹H NMR (400 MHz, CD₃OD) δ 8.26- 8.20 (m, 2H), 7.77-7.68 (m, 2H), 7.35-7.30 (m, 3H), 5.17-5.10 (m, 1H), 3.95 (m, 1H), 3.48 (m, 1H), 3.41 (m, 1H), 3.38 (m, 2H), 2.23 (m, 1H), 1.60-1.54 (m, 1H), 1.44 (m, 6H), 1.35 (m, 1H), 0.94 (m, 3H). |
| | N-(4-((3R,4R)-3-amino-4- | | |
| | ethylpyrrolidin-1-yl)-7-fluoro-1- | | |
| | isopropyl-1H-indazol-5-yl)-1- | | |
| | (2,6-difluorophenyl)-6-oxo-1,6- | | |
| | dihydropyridazine-3- | | |
| | carboxamide | | |
| Example 217 | 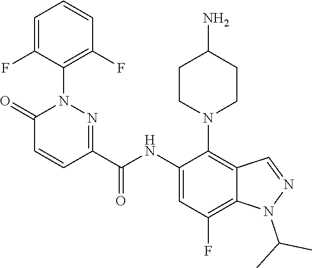 [SMILES: CC(C)n1ncc2c(N3CCC(N)CC3)c(NC(=O)c3ccc(=O)n(-c4c(F)cccc4F)n3)cc(F)c21] | HCOOH Salt | LC-MS m/z: 526.2 (M + 1)⁺. 1H NMR (400 MHz, CD₃OD) δ 8.31 (d, J = 2.0 Hz, 1H), 8.29-8.18 (m, 2H), 7.69 (tt, J = 8.6, 6.2 Hz, 1H), 7.41-7.26 (m, 3H), 5.13 (h, J = 6.6 Hz, 1H), 3.26-3.10 (m, 4H), 3.05 (dd, J = 8.3, 4.3 Hz, 1H), 1.93-1.82 (m, 2H), 1.55 (d, J = 6.6 Hz, 6H), 1.50 (m, 2H). |
| | N-(4-(4-aminopiperidin-1-yl)-7- | | |
| | fluoro-1-isopropyl-1H-indazol-5- | | |
| | yl)-1-(2,6-difluorophenyl)-6-oxo- | | |
| | 1,6-dihydropyridazine-3- | | |
| | carboxamide | | |
| Example 218 | 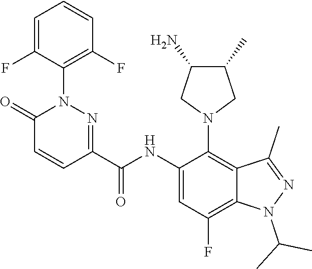 [SMILES: Cc1nn(C(C)C)c2c(F)cc(NC(=O)c3ccc(=O)n(-c4c(F)cccc4F)n3)c(N3C[C@@H](C)[C@@H](N)C3)c12] | HCl Salt | LC-MS m/z: 540.3 (M + 1)⁺. ¹H NMR (400 MHz, CD₃OD) δ 8.24- 8.19 (m, 1H), 7.73-7.58 (m, 2H), 7.36-7.27 (m, 3H), 5.09-5.01 (m, 1H), 3.84-3.73 (m, 1H), 3.69-3.61 (m, 1H), 3.59-3.51 (m, 1H), 3.34- 3.32 (m, 1H), 3.12-3.06 (m, 1H), 2.68-2.59 (m, 4H), 1.55-1.50 (m, 6H), 1.06-0.99 (m, 3H). |
| | N-(4-((3R,4R)-3-amino-4- | | |
| | methylpyrrolidin-1-yl)-7-fluoro- | | |
| | 1-isopropyl-3-methyl-1H- | | |
| | indazol-5-yl)-1-(2,6- | | |
| | difluorophenyl)-6-oxo-1,6- | | |
| | dihydropyridazine-3- | | |
| | carboxamide | | |
| Example 219 | 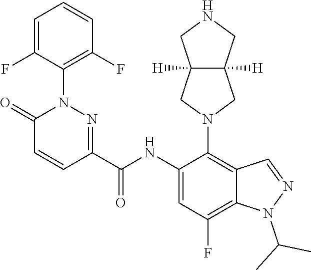 [SMILES: CC(C)n1ncc2c(N3C[C@H]4CNC[C@H]4C3)c(NC(=O)c3ccc(=O)n(-c4c(F)cccc4F)n3)cc(F)c21] | HCOOH Salt | LC-MS m/z: 538.2 (M + 1)⁺. ¹H NMR (400 MHz, CD₃OD) δ 8.34 (d, J = 1.7 Hz, 1H), 8.24 (d, J = 9.8 Hz, 1H), 7.91 (d, J = 13.3 Hz, 1H), 7.76-7.67 (m, 1H), 7.40-7.31 (m, 3H), 5.16 (td, J = 6.7, 13.3 Hz, 1H), 3.55-3.49 (m, 2H), 3.44-3.36 (m, 3H), 3.28-3.21 (m, 4H), 3.05-2.96 (m, 2H), 1.57 (d, J = 6.6 Hz, 6H). |
| | 1-(2,6-difluorophenyl)-N-(7- | | |
| | fluoro-4-((3aR,6aS)- | | |
| | hexahydropyrrolo[3,4-c]pyrrol- | | |
| | 2(1H)-yl)-1-isopropyl-1H- | | |
| | indazol-5-yl)-6-oxo-1,6- | | |
| | dihydropyridazine-3- | | |
| | carboxamide | | |

### Example 220: (R)—N-(4-(3-amino-3-methylpyrrolidin-1-yl)-2-isopropyl-2H-indazol-5-yl)-1-(2,6-difluorophenyl)-6-oxo-1,6-dihydropyridazine-3-carboxamide

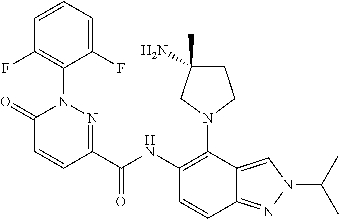 [SMILES: CC(C)n1cc2c(N3CC[C@@](C)(N)C3)c(NC(=O)c3ccc(=O)n(-c4c(F)cccc4F)n3)ccc2n1]

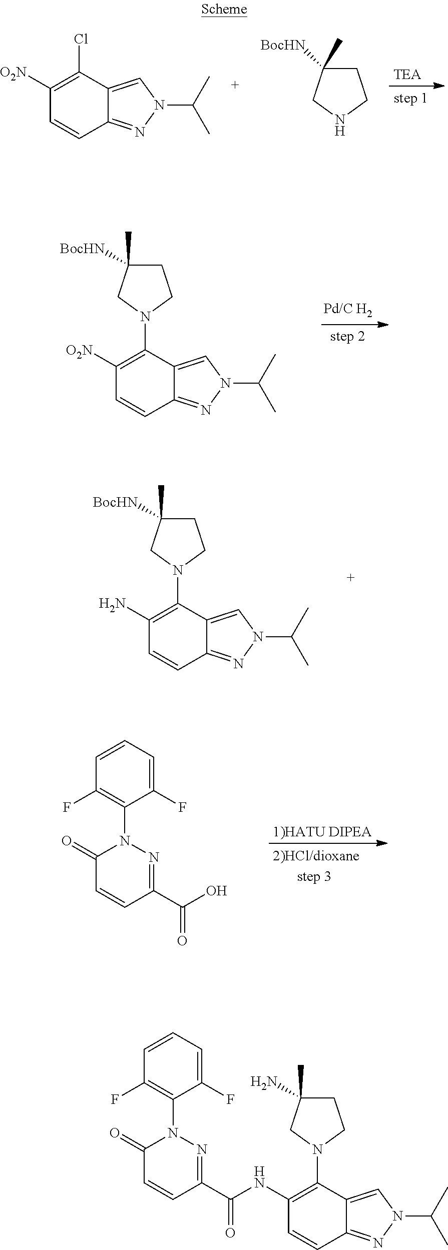 [SMILES: CC(C)n1cc2c(Cl)c([N+](=O)[O-])ccc2n1 ; CC(C)(C)OC(=O)N[C@]1(C)CCNC1 ; CC(C)n1cc2c(N3CC[C@@](C)(NC(=O)OC(C)(C)C)C3)c([N+](=O)[O-])ccc2n1 ; CC(C)n1cc2c(N3CC[C@@](C)(NC(=O)OC(C)(C)C)C3)c(N)ccc2n1 ; O=C(O)c1ccc(=O)n(-c2c(F)cccc2F)n1 ; CC(C)n1cc2c(N3CC[C@@](C)(N)C3)c(NC(=O)c3ccc(=O)n(-c4c(F)cccc4F)n3)ccc2n1]

### Step 1: tert-butyl (R)-(1-(2-isopropyl-5-nitro-2H-indazol-4-yl)-3-methylpyrrolidin-3-yl)carbamate

To a solution of 4-chloro-5-nitro-2-(prop-2-yl)indazole (240 mg, 1.001 mmol) in dioxane (10 mL) were added 2-methylpropan-2-yl {[(3R)-3-methyltetrahydro-1H-pyrrol-3-yl]amino}methanoate (200.56 mg, 1.001 mmol) and Et3N (0.418 mL, 3.004 mmol), and the reaction was stirred at 100° C. for 5 hr, as a brown suspension. LCMS showed the reaction finished. The reaction mixture was concentrated in vacuo to afford tert-butyl (R)-(1-(2-isopropyl-5-nitro-2H-indazol-4-yl)-3-methylpyrrolidin-3-yl)carbamate (405 mg) as a yellow solid.

LC-MS m/z: 404.5 (M+1)⁺.

### Step 2: tert-butyl (R)-(1-(5-amino-2-isopropyl-2H-indazol-4-yl)-3-methylpyrrolidin-3-yl)carbamate

To a solution of 2-methylpropan-2-yl {[(3R)-3-methyl-1-[5-nitro-2-(prop-2-yl)indazol-4-yl]tetrahydro-1H-pyrrol-3-yl]amino}methanoate (405 mg, 1.004 mmol) in MeOH 20 mL, was added Pd/C 10% (100 mg, 0.940 mmol) and under hydrogen for 15 h at r.t (60 Psi) as a black suspension, Target compound MS was detected by LC-MS. The mixture of reaction was filtered, the filtrate was concentrated to dryness in vacuo affording tert-butyl (R)-(1-(5-amino-2-isopropyl-2H-indazol-4-yl)-3-methylpyrrolidin-3-yl)carbamate (353 mg) as a yellow solid which was directly used to the next reaction.

LC-MS m/z: 374.5 (M+1)⁺.

### Step 3: (R)—N-(4-(3-amino-3-methylpyrrolidin-1-yl)-2-isopropyl-2H-indazol-5-yl)-1-(2,6-difluoro phenyl)-6-oxo-1,6-dihydropyridazine-3-carboxamide

To a solution of 1-(2,6-difluorophenyl)-6-oxo-1,2-diazine-3-carboxylic acid (150 mg, 0.595 mmol) (150 mg, 0.43 mmol, 1.00 eq.), 2-methylpropan-2-yl{[(3R)-1-[5-amino-2-(prop-2-yl)indazol-4-yl]-3-methyltetrahydro-1H-pyrrol-3-yl]amino}methanoate (244.38 mg, 0.654 mmol) in ACN (10 mL) was added ethyldiisopropylamine (0.3 mL, 1.815 mmol), follow by HATU (339.17 mg, 0.892 mmol) and stirred for 2 h to get a black suspension. LC-MS: showed most of the start material was consumed and one peak was detected. The reaction was diluted with H2O 50 mL and extracted with EA 30 mL*2. The combined organic layers were dried over Na2SO4, filtered and concentrated. The residue was purified by FLASH column (SiO₂, EA in PE 60%, COMBI FLASH NEXTAGEN 300) to afford intermediate 120 mg as yellow solid. To intermediate was added 10 mL HCl/dioxane(4 mol/L), and stirred 3 h as a yellow suspension. The reaction mixture was concentrated to afford Example 220 (22.32 mg) as a yellow solid.

LC-MS m/z: 508.5 (M+1)⁺.

¹H NMR (400 MHz, CD₃OD) δ 8.47 (s, 1H), 8.25 (d, J=9.8 Hz, 1H), 7.90 (d, J=9.2 Hz, 1H), 7.69 (dd, J=10.6, 4.2 Hz, 1H), 7.32 (q, J=8.9 Hz, 4H), 3.63 (dd, J=16.5, 8.1 Hz, 2H), 3.45 (dd, J=20.6, 11.4 Hz, 4H), 2.02 (s, 1H), 1.94 (s, 1H), 1.64 (t, J=8.8 Hz, 6H), 1.31 (s, 2H), 1.27 (s, 3H).

| No. | Structure | Salt form | MS and 1HNMR |
|---|---|---|---|
| Example 221 | 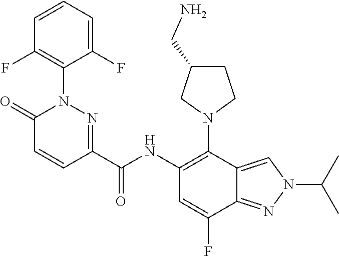 [SMILES: CC(C)n1cc2c(N3CC[C@@H](CN)C3)c(NC(=O)c3ccc(=O)n(-c4c(F)cccc4F)n3)cc(F)c2n1] | HCl Salt | LC-MS m/z: 526.5 (M + 1)⁺. ¹H NMR (400 MHz, CD₃OD) δ 8.59 (s, 1H), 8.25 (d, J = 9.9 Hz, 1H), 7.97 (s, 1H), 7.79-7.60 (m, 1H), 7.41- 7.28 (m, 3H), 3.72 (dd, J = 27.3, 5.3 Hz, 1H), 3.62-3.49 (m, 2H), 3.15 (s, 1H), 2.88 (s, 2H), 2.63 (s, 1H), 2.21 (s, 1H), 1.67 (d, J = 6.7 Hz, 6H), 1.58 (d, J = 6.3 Hz, 1H), 1.32 (dd, J = 19.9, 12.3 Hz, 1H). |
| | (S)-N-(4-(3- | | |
| | (aminomethyl)pyrrolidin-1-yl)- | | |
| | 7-fluoro-2-isopropyl-2H- | | |
| | indazol-5-yl)-1-(2,6- | | |
| | difluorophenyl)-6-oxo-1,6- | | |
| | dihydropyridazine-3- | | |
| | carboxamide | | |
| Example 222 | 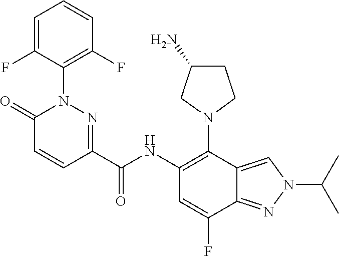 [SMILES: CC(C)n1cc2c(N3CC[C@@H](N)C3)c(NC(=O)c3ccc(=O)n(-c4c(F)cccc4F)n3)cc(F)c2n1] | HCl Salt | LC-MS m/z: 512.2 (M + 1)⁺. ¹H NMR (500 MHz, CD₃OD) δ 8.56 (d, J = 2.6 Hz, 1H), 8.23 (d, J = 9.9 Hz, 1H), 7.98 (d, J = 12.7 Hz, 1H), 7.77- 7.64 (m, 1H), 7.38-7.29 (m, 3H), 4.91 (br s, 1H), 3.64-3.58 (m, 1H), 3.58-3.53 (m, 1H), 3.47 (q, J = 7.8 Hz, 1H), 3.25-3.19 (m, 1H), 3.15 (dd, J = 5.0, 9.2 Hz, 1H), 2.26-2.12 (m, 1H), 1.87-1.73 (m, 1H), 1.67 (d, J = 6.7 Hz, 6H) |
| | (R)-N-(4-(3-aminopyrrolidin-1- | | |
| | yl)-7-fluoro-2-isopropyl-2H- | | |
| | indazol-5-yl)-1-(2,6- | | |
| | difluorophenyl)-6-oxo-1,6- | | |
| | dihydropyridazine-3- | | |
| | carboxamide | | |
| Example 223 | 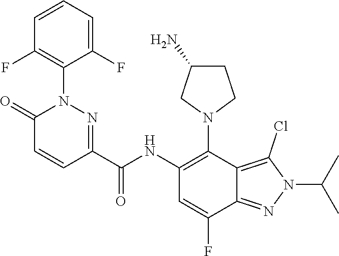 [SMILES: CC(C)n1nc2c(F)cc(NC(=O)c3ccc(=O)n(-c4c(F)cccc4F)n3)c(N3CC[C@@H](N)C3)c2c1Cl] | HCl Salt | LC-MS m/z: 546.2 (M + 1)⁺. ¹H NMR (400 MHz, CD₃OD) δ 8.24 (d, J = 9.9 Hz, 1H), 8.11 (d, J = 12.6 Hz, 1H), 7.82-7.61 (m, 1H), 7.35 (dd, J = 14.4, 9.0 Hz, 3H), 5.15 (dt, J = 13.4, 6.7 Hz, 1H), 3.72-3.49 (m, 4H), 3.39-3.34 (m, 2H), 2.31 (dd, J = 13.0, 4.8 Hz, 1H), 2.10-1.86 (m, 1H), 1.62 (dt, J = 8.3, 4.1 Hz, 6H). |
| | (R)-N-(4-(3-aminopyrrolidin-1- | | |
| | yl)-3-chloro-7-fluoro-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 224 | 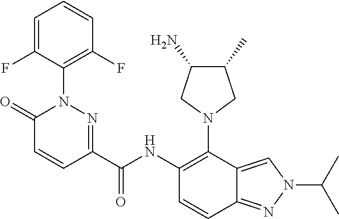 [SMILES: CC(C)n1cc2c(N3C[C@@H](C)[C@@H](N)C3)c(NC(=O)c3ccc(=O)n(-c4c(F)cccc4F)n3)ccc2n1] | HCOOH Salt | LC-MS m/z: 508.3 (M + 1)⁺. ¹H NMR (400 MHz, CD₃OD) δ 8.47 (d, J = 1.0 Hz, 1H), 8.22 (d, J = 9.9 Hz, 1H), 7.75-7.58 (m, 2H), 7.38-7.19 (m, 4H), 4.85-4.78 (m, 1H), 3.95 (dd, J = 10.4, 6.1 Hz, 1H), 3.75 (td, J = 6.1, 3.6 Hz, 1H), 3.57 (dd, J = 9.2, 7.4 Hz, 1H), 3.48-3.35 (m, 2H), 2.53 (hept, J = 7.3 Hz, 1H), 1.64 (d, J = 6.7 Hz, 6H), 1.12 (d, J = 7.1 Hz, 3H). |
| | N-(4-((3R,4R)-3-amino-4- | | |
| | methylpyrrolidin-1-yl)-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 225 | 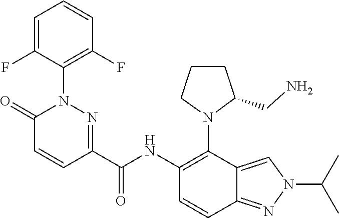 [SMILES: CC(C)n1cc2c(N3CCC[C@@H]3CN)c(NC(=O)c3ccc(=O)n(-c4c(F)cccc4F)n3)ccc2n1] | HCOOH Salt | LC-MS m/z: 508.3 (M + 1)⁺. ¹H NMR (400 MHz, CD₃OD) δ 8.43 (s, 1H), 8.22 (d, J = 9.9 Hz, 1H), 8.09 (d, J = 9.3 Hz, 1H), 7.70 (tt, J = 8.6, 6.2 Hz, 1H), 7.49 (d, J = 9.2 Hz, 1H), 7.39-7.26 (m, 3H), 4.85 (q, J = 6.7 Hz, 2H), 4.06 (qd, J = 7.3, 4.0 Hz, 1H), 3.36-3.26 (m, 3H), 3.15 (dt, J = 9.1, 7.3 Hz, 1H), 2.77 (dd, J = 12.6, 4.0 Hz, 1H), 2.68 (dd, J = 12.7, 7.6 Hz, 1H), 2.35 (dq, J = 14.3, 7.3 Hz, 1H), 2.06-1.95 (m, 1H), 1.84-1.67 (m, 2H), 1.65 (d, J = 6.7 Hz, 6H). |
| | (R)-N-(4-(2- | | |
| | (aminomethyl)pyrrolidin-1-yl)- | | |
| | 2-isopropyl-2H-indazol-5-yl)-1- | | |
| | (2,6-difluorophenyl)-6-oxo-1,6- | | |
| | dihydropyridazine-3- | | |
| | carboxamide | | |
| Example 226 | 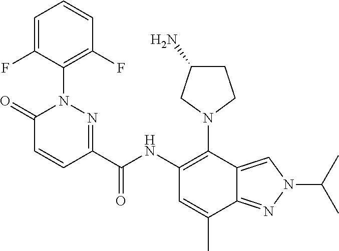 [SMILES: Cc1cc(NC(=O)c2ccc(=O)n(-c3c(F)cccc3F)n2)c(N2CC[C@@H](N)C2)c2cn(C(C)C)nc12] | HCl Salt | LC-MS m/z: 508.3 (M + 1)⁺. ¹H NMR (500 MHz, CD₃OD) δ 8.66 (s, 1H), 8.24-8.17 (m, 1H), 7.71- 7.61 (m, 2H), 7.35-7.26 (m, 3H), 4.95-4.89 (m, 1H), 3.85-3.73 (m, 2H), 3.71-3.63 (m, 1H), 3.47-3.34 (m, 2H), 2.56-2.52 (m, 3H), 2.38- 2.26 (m, 1H), 2.06-1.97 (m, 1H), 1.70-1.66 (m, 6H). |
| | (R)-N-(4-(3-aminopyrrolidin-1- | | |
| | yl)-2-isopropyl-7-methyl-2H- | | |
| | indazol-5-yl)-1-(2,6- | | |
| | difluorophenyl)-6-oxo-1,6- | | |
| | dihydropyridazine-3- | | |
| | carboxamide | | |
| Example 227 | 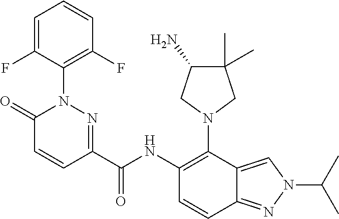 [SMILES: CC(C)n1cc2c(N3C[C@H](N)C(C)(C)C3)c(NC(=O)c3ccc(=O)n(-c4c(F)cccc4F)n3)ccc2n1] | HCl Salt | LC-MS m/z: 522.3 (M + 1)⁺. ¹H NMR (400 MHz, CD₃OD) δ 8.47 (s, 1H), 8.22 (d, J = 9.8 Hz, 1H), 7.75 (d, J = 9.1 Hz, 1H), 7.70-7.62 (m, 1H), 7.32-7.24 (m, 4H), 4.85-4.80 (m, 1H), 3.89-3.82 (m, 1H), 3.42 (d, J = 8.9 Hz, 1H), 3.30-3.25 (m, 3H), 1.67-1.67 (m, 1H), 1.63 (d, J = 6.7 Hz, 5H), 1.06 (d, J = 3.9 Hz, 6H) |
| | (R)-N-(4-(4-amino-3,3- | | |
| | dimethylpyrrolidin-1-yl)-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 228 | 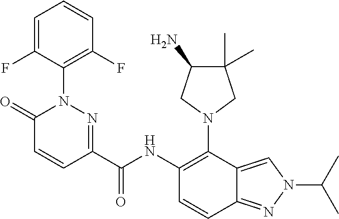 [SMILES: CC(C)n1cc2c(N3C[C@@H](N)C(C)(C)C3)c(NC(=O)c3ccc(=O)n(-c4c(F)cccc4F)n3)ccc2n1] | HCl Salt | LC-MS m/z: 522.3 (M + 1)⁺. ¹H NMR (400 MHz, CD₃OD) δ 8.45 (s, 1H), 8.23 (d, J = 9.9 Hz, 1H), 7.73 (d, J = 9.2 Hz, 1H), 7.70-7.61 (m, 1H), 7.34-7.25 (m, 4H), 4.85-4.78 (m, 1H), 3.95-3.85 (m, 1H), 3.49-3.42 (m, 1H), 3.39-3.32 (m, 2H), 3.37- 3.32 (m, 1H), 1.63 (d, J = 6.8 Hz, 6H), 1.09 (s, 6H) |
| | (S)-N-(4-(4-amino-3,3- | | |
| | dimethylpyrrolidin-1-yl)-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 229 | 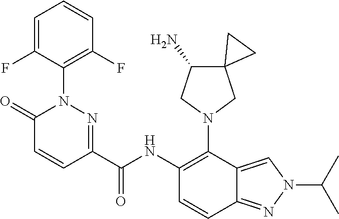 [SMILES: CC(C)n1cc2c(N3C[C@H](N)C4(CC4)C3)c(NC(=O)c3ccc(=O)n(-c4c(F)cccc4F)n3)ccc2n1] | HCOOH salt | LC-MS m/z: 520.3 (M + 1)⁺ ¹H NMR (400 MHz, CD₃OD): δ 8.53 (s, 1H), 8.48 (s, 1H), 8.21 (d, J = 9.8 Hz, 1H), 7.75 (d, J = 9.2 Hz, 1H), 7.66 (ddd, J = 14.7, 8.6, 6.2 Hz, 1H), 7.29 (dq, J = 8.8, 4.5, 3.9 Hz, 4H), 4.83- 4.77 (m, 1H), 4.04 (dd, J = 10.1, 5.6 Hz, 1H), 3.84 (d, J = 9.0 Hz, 1H), 3.48 (dd, J = 10.1, 3.1 Hz, 1H), 3.22 (dd, J = 5.7, 3.1 Hz, 1H), 3.02 (d, J = 9.1 Hz, 1H), 1.63 (d, J = 6.7 Hz, 6H), 0.91 (dt, J = 8.3, 4.4 Hz, 1H), 0.74-0.53 (m, 3H). |
| | (R)-N-(4-(7-amino-5- | | |
| | azaspiro[2.4]heptan-5-yl)-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 230 | 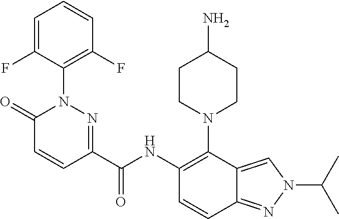 [SMILES: CC(C)n1cc2c(N3CCC(N)CC3)c(NC(=O)c3ccc(=O)n(-c4c(F)cccc4F)n3)ccc2n1] | HCl Salt | LC-MS m/z: 508.1 (M + 1)⁺ ¹H NMR (400 MHz, CD₃OD) δ 8.59 (s, 1H), 8.24 (d, J = 9.9 Hz, 1H), 8.12 (br d, J = 9.1 Hz, 1H), 7.76-7.63 (m, 1H), 7.49 (d, J = 9.3 Hz, 1H), 7.40-7.28 (m, 3H), 4.89-4.81 (m, 1H), 3.41- 3.35 (m, 2H), 3.29 (br s, 1H), 3.23- 3.12 (m, 2H), 1.96 (br s, 2H), 1.78- 1.69 (m, 2H), 1.67 (d, J = 6.8 Hz, 6H) |
| | N-(4-(4-aminopiperidin-1-yl)-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 231 | 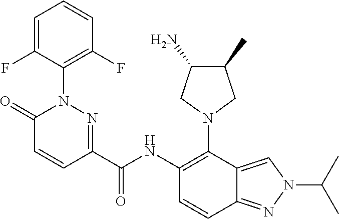 [SMILES: CC(C)n1cc2c(N3C[C@H](C)[C@@H](N)C3)c(NC(=O)c3ccc(=O)n(-c4c(F)cccc4F)n3)ccc2n1] | HCOOH Salt | LC-MS m/z: 508.6 (M + 1)⁺. ¹H NMR (400 MHz, CD₃OD) δ 8.24 (d, J = 9.7 Hz, 2H), 7.87 (d, J = 9.0 Hz, 1H), 7.68 (tt, J = 8.5, 6.2 Hz, 1H), 7.38-7.26 (m, 4H), 3.86 (dt, J = 9.3, 7.3 Hz, 2H), 3.46 (dd, J = 9.9, 6.1 Hz, 1H), 3.24 (q, J = 6.1 Hz, 1H), 3.14 (dd, J = 9.3, 5.6 Hz, 1H), 2.24 (p, J = 6.3 Hz, 1H), 1.55 (d, J = 6.6 Hz, 6H), 1.07 (d, J = 6.9 Hz, 3H). |
| | N-(4-((3R,4S)-3-amino-4- | | |
| | methylpyrrolidin-1-yl)-2- | | |
| | isopropyl-2H-indazol-5-yl)-1- | | |
| | (2,6-difluorophenyl)-6-oxo-1,6- | | |
| | dihydropyridazine-3- | | |
| | carboxamide | | |
| Example 232 | 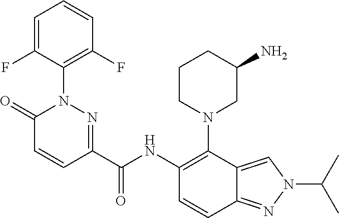 [SMILES: CC(C)n1cc2c(N3CCC[C@@H](N)C3)c(NC(=O)c3ccc(=O)n(-c4c(F)cccc4F)n3)ccc2n1] | HCl Salt | LC-MS m/z: 508.3 (M + 1)⁺ ¹H NMR (400 MHz, CD₃OD) δ 8.51 (s, 1H), 8.41 (br s, 1H), 8.27 (d, J = 9.9 Hz, 1H), 7.80-7.67 (m, 1H), 7.56 (d, J = 9.3 Hz, 1H), 7.45-7.30 (m, 3H), 4.90-4.82 (m, 1H), 3.30- 3.12 (m, 3H), 3.08 (br s, 1H), 2.97 (br d, J = 11.6 Hz, 1H), 1.91 (br d, J = 10.8 Hz, 1H), 1.79 (br d, J = 13.6 Hz, 1H), 1.66 (d, J = 6.7 Hz, 6H), 1.62-1.50 (m, 1H), 1.46-1.34 (m, 1H) |
| | (R)-N-(4-(3-aminopiperidin-1- | | |
| | yl)-2-isopropyl-2H-indazol-5-yl)- | | |
| | 1-(2,6-difluorophenyl)-6-oxo- | | |
| | 1,6-dihydropyridazine-3- | | |
| | carboxamide | | |
| Example 233 | 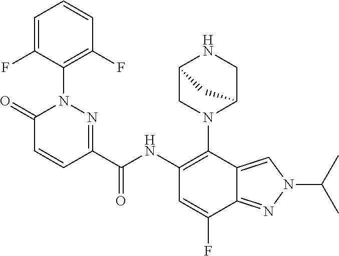 [SMILES: CC(C)n1cc2c(N3C[C@@H]4C[C@H]3CN4)c(NC(=O)c3ccc(=O)n(-c4c(F)cccc4F)n3)cc(F)c2n1] | HCl Salt | LC-MS m/z: 524.2 (M + 1)⁺ ¹H NMR (400 MHz, CD₃OD) δ 8.60 (d, J = 2.9 Hz, 1H), 8.23 (d, J = 9.8 Hz, 1H), 7.74-7.62 (m, 1H), 7.47 (d, J = 11.9 Hz, 1H), 7.37-7.26 (m, 3H), 4.92-4.89 (m, 1H), 4.48 (s, 2H), 3.82 (dd, J = 2.5, 11.1 Hz, 1H), 3.55- 3.50 (m, 1H), 3.47 (dd, J = 1.5, 11.3 Hz, 1H), 3.37-3.34 (m, 1H), 2.23 (br d, J = 11.3 Hz, 1H), 1.97 (br d, J = 11.4 Hz, 1H), 1.67 (d, J = 6.7 Hz, 6H). |
| | N-(4-((1S,4S)-2,5- | | |
| | diazabicyclo[2.2.1]heptan-2-yl)- | | |
| | 7-fluoro-2-isopropyl-2H- | | |
| | indazol-5-yl)-1-(2,6- | | |
| | difluorophenyl)-6-oxo-1,6- | | |
| | dihydropyridazine-3- | | |
| | carboxamide | | |

### Example 234: N-[4-[(3R,4R)-3-amino-4-methyl-pyrrolidin-1-yl]-2-tert-butyl-indazol-5-yl]-1-(2,6-difluorophenyl)-6-oxo-pyridazine-3-carboxamide

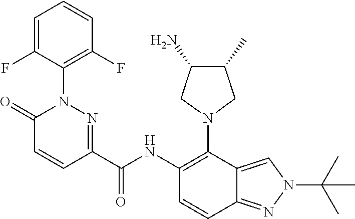 [SMILES: C[C@@H]1CN(c2c(NC(=O)c3ccc(=O)n(-c4c(F)cccc4F)n3)ccc3nn(C(C)(C)C)cc23)C[C@@H]1N]

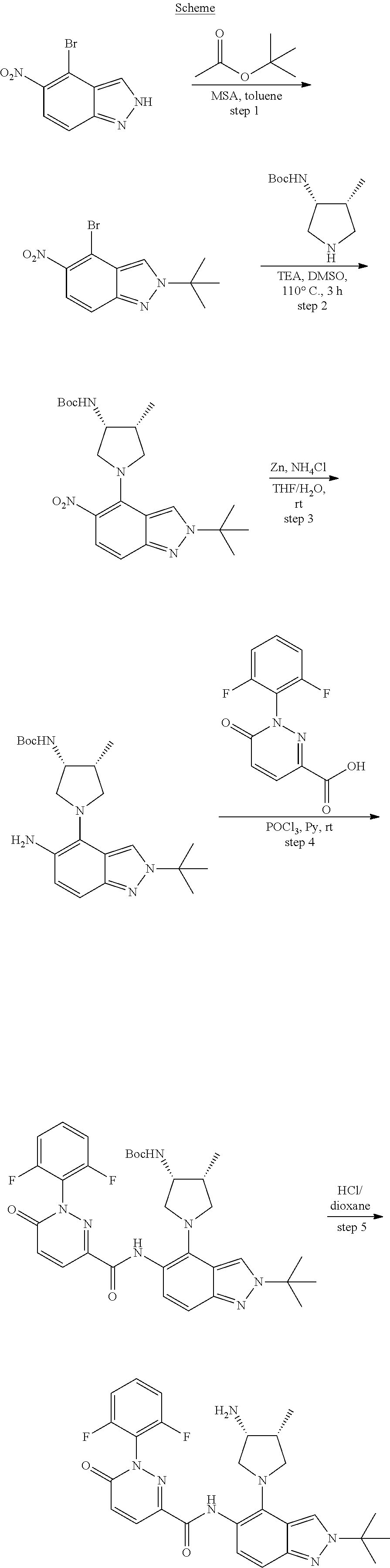

### Step 1: 4-bromo-2-tert-butyl-5-nitro-indazole

To a solution of 4-bromo-5-nitro-2H-indazole (10 g, 41.32 mmol, 1 eq) in toluene (30 mL) was added tert-butyl acetate (95.99 g, 826.35 mmol, 110.84 mL, 20 eq) and MSA (3.97 g, 41.32 mmol, 2.94 mL, 1 eq). The mixture was stirred at 70° C. for 48 hr. LCMS showed the desired product was formed as a major peak. The reaction mixture was poured into saturated NaHCO₃ (50 mL), extracted with EA (40 mL×3). The combined organic phases were dried over Na₂SO₄, filtered. The filtrate was concentrated in vacuum. The residue was used next step directly without further purification. 4-bromo-2-tert-butyl-5-nitro-indazole (12 g, Yield: 97.42%) was obtained as a white solid.

LC-MS m/z:298.0 (M+1)⁺

### Step 2: tert-butyl N-[(3R,4R)-1-(2-tert-butyl-5-nitro-indazol-4-yl)-4-methyl-pyrrolidin-3-yl]carbamate

To a solution of 4-bromo-2-tert-butyl-5-nitro-indazole (9 g, 30.19 mmol, 1 eq) in DMSO (90 mL) was added TEA (9.16 g, 90.56 mmol, 12.61 mL, 3 eq) and tert-butyl N-[(3R,4R)-4-methylpyrrolidin-3-yl]carbamate (6.05 g, 30.19 mmol, 1 eq). The mixture was stirred at 100° C. for 2 hr. LCMS showed the desired product was formed as a major peak. The reaction mixture was poured into water (400 mL), the formed solid was filtered and collected. The solid was dissolved in EA (500 mL), dried over Na₂SO₄, filtered. The filtrate was concentrated in vacuum. The residue was used next step directly without further purification. tert-butyl N-[(3R,4R)-1-(2-tert-butyl-5-nitro-indazol-4-yl)-4-methyl-pyrrolidin-3-yl]carbamate (11 g, Yield: 87.28%) was obtained as a yellow solid.

LC-MS m/z:418.4 (M+1)⁺

### Step 3: tert-butyl N-[(3R,4R)-1-(5-amino-2-tert-butyl-indazol-4-yl)-4-methyl-pyrrolidin-3-yl]carbamate

To a solution of tert-butyl N-[(3R,4R)-1-(2-tert-butyl-5-nitro-indazol-4-yl)-4-methyl-pyrrolidin-3-yl]carbamate (11 g, 26.35 mmol, 1 eq) in THF (120 mL) and H₂O (60 mL) was added NH₄Cl (11.27 g, 210.78 mmol, 8 eq) and Zn (13.89 g, 212.42 mmol, 8.06 eq) in portions keeping temperature blew 25° C. using ice water. The mixture was stirred at 25° C. for 1 hr. LCMS showed the desired product was formed as a major peak. The reaction mixture was filtered. The filtrate was poured into water (60 mL), extracted with EA (60 mL×3). The combined organic phases were dried over Na₂SO₄, filtered. The filtrate was concentrated in vacuo. Remained Zn was dealt with 1 N HCl, then neutralized with 1 N NaOH and discarded. The residue was used next step directly without further purification. tert-butyl N-[(3R,4R)-1-(5-amino-2-tert-butyl-indazol-4-yl)-4-methyl-pyrrolidin-3-yl]carbamate (10 g, Yield: 97.94%) was obtained as a green solid.

LC-MS m/z:388.3 (M+1)⁺

### Step 4: tert-butyl N-[(3R,4R)-1-[2-tert-butyl-5-[[1-(2,6-difluorophenyl)-6-oxo-pyridazine-3-carbonyl]amino]indazol-4-yl]-4-methyl-pyrrolidin-3-yl]carbamate

To a solution of tert-butyl N-[(3R,4R)-1-(5-amino-2-tert-butyl-indazol-4-yl)-4-methyl-pyrrolidin-3-yl]carbamate (10 g, 25.81 mmol, 1 eq) and 1-(2,6-difluorophenyl)-6-oxo-pyridazine-3-carboxylic acid (6.51 g, 25.81 mmol, 1 eq) in CH₃CN (200 mL) was added NMI (6.36 g, 77.42 mmol, 6.17 mL, 3 eq) and TCFH (8.69 g, 30.97 mmol, 1.2 eq). The mixture was stirred at 25° C. for 3 hr. LCMS showed the desired product was formed as a major peak. The reaction mixture was poured into water (400 mL), extracted with EA (100 mL×3). The combined organic phases were dried over Na₂SO₄, filtered. The filtrate was concentrated in vacuo. The residue was purified by flash silica gel chromatography (ISCO®; 100 g SepaFlash® Silica Flash Column, Eluent of 0^{˜}40% Ethyl acetate/Petroleum ethergradient @200 mL/min). The product was dissolved in PE/EA (5:1, 50 ml) and stirring for 1 h. The mixture was filtered. The solid was collected. tert-butyl N-[(3R,4R)-1-[2-tert-butyl-5-[[1-(2,6-difluorophenyl)-6-oxo-pyridazine-3-carbonyl]amino]indazol-4-yl]-4-methyl-pyrrolidin-3-yl]carbamate (11 g, Yield: 68.57%) was obtained as a yellow solid.

LC-MS m/z:622.3 (M+1)⁺

### Step 5: N-[4-[(3R,4R)-3-amino-4-methyl-pyrrolidin-1-yl]-2-tert-butyl-indazol-5-yl]-1-(2,6-difluoro phenyl)-6-oxo-pyridazine-3-carboxamide

To a solution of tert-butyl N-[(3R,4R)-1-[2-tert-butyl-5-[[1-(2,6-difluorophenyl)-6-oxo-pyridazine-3-carbonyl]amino]indazol-4-yl]-4-methyl-pyrrolidin-3-yl]carbamate (11 g, 17.69 mmol, 1 eq) in MeOH (20 mL) was added HCl/MeOH (4 M, 55.00 mL, 12.43 eq). The mixture was stirred at 25° C. for 62 hr. LCMS showed the desired product was formed as a major peak. The reaction mixture was concentrated in vacuum. The residue was dissolved in CH₃CN/H₂O (20 mL/50 mL) and lyophilized. The residue was used next step directly without further purification.

### Example 234 (9.23 g, Yield: 100.00%) was Obtained as a White Solid

LC-MS m/z:492.1 (M+1)⁺

1H NMR (400 MHz, CD₃OD) δ 8.65 (s, 1H), 8.22 (d, J=9.8 Hz, 1H), 7.75-7.63 (m, 2H), 7.37-7.24 (m, 4H), 4.04 (dd, J=6.0, 10.7 Hz, 1H), 3.88-3.88 (m, 1H), 3.88-3.81 (m, 1H), 3.71-3.63 (m, 1H), 3.60-3.48 (m, 2H), 2.62 (td, J=7.4, 14.6 Hz, 1H), 1.81 (s, 9H), 1.18 (d, J=7.0 Hz, 3H).

| No. | Structure | Salt form | MS and ¹HNMR |
|---|---|---|---|
| Example 235 | 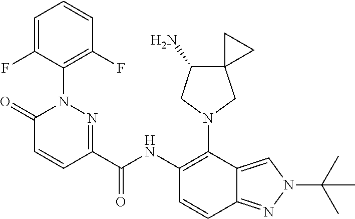 [SMILES: CC(C)(C)n1cc2c(N3C[C@H](N)C4(CC4)C3)c(NC(=O)c3ccc(=O)n(-c4c(F)cccc4F)n3)ccc2n1] | HCOOH Salt | LC-MS m/z: 534.3 (M + 1)⁺ ¹H NMR (400 MHz, CD₃OD) δ 8.47 (s, 1H), 8.21 (d, J = 9.8 Hz, 1H), 7.79 (d, J = 9.2 Hz, 1H), 7.66 (tt, J = 8.6, 6.2 Hz, 1H), 7.38-7.22 (m, 4H), 4.03 (dd, J = 10.1, 5.7 Hz, 1H), 3.84 (d, J = 9.0 Hz, 1H), 3.49 (dd, J = 10.1, 3.3 Hz, 1H), 3.24 (dd, J = 5.8, 3.3 Hz, 1H), 2.99 (d, J = 9.0 Hz, 1H), 1.74 (s, 9H), 0.92 (dt, J = 9.8, 5.2 Hz, 1H), 0.66 (dtd, J = 19.1, 9.6, 5.6 Hz, 2H), 0.55 (dt, J = 10.4, 5.0 Hz, 1H). |
| | (R)-N-(4-(7-amino-5- | | |
| | azaspiro[2.4]heptan-5-yl)-2- | | |
| | (tert-butyl)-2H-indazol-5- | | |
| | yl)-1-(2,6-difluorophenyl)-6- | | |
| | oxo-1,6-dihydropyridazine- | | |
| | 3-carboxamide | | |
| Example 236 | 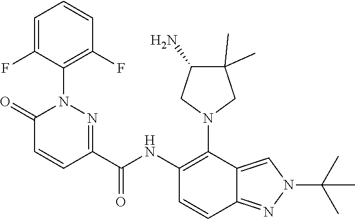 [SMILES: CC1(C)CN(c2c(NC(=O)c3ccc(=O)n(-c4c(F)cccc4F)n3)ccc3nn(C(C)(C)C)cc23)C[C@@H]1N] | HCl Salt | LC-MS m/z: 536.3 (M + 1)⁺ ¹H NMR (500 MHz, CD₃OD) δ 8.60-8.55 (m, 1H), 8.25-8.20 (m, 1H), 7.78-7.73 (m, 1H), 7.70-7.63 (m, 1H), 7.33-7.26 (m, 4H), 4.06-3.98 (m, 1H), 3.61-3.57 (m, 1H), 3.47-3.42 (m, 2H), 3.35-3.33 (m, 1H), 1.80-1.76 (m, 9H), 1.19-1.11 (m, 6H). |
| | (R)-N-(4-(4-amino-3,3- | | |
| | dimethylpyrrolidin-1-yl)-2- | | |
| | (tert-butyl)-2H-indazol-5-yl)- | | |
| | 1-(2,6-difluorophenyl)-6- | | |
| | oxo-1,6-dihydropyridazine- | | |
| | 3-carboxamide | | |
| Example 237 | 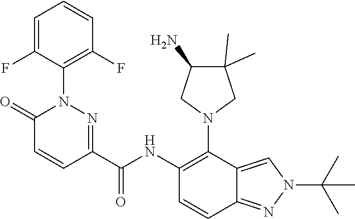 [SMILES: CC1(C)CN(c2c(NC(=O)c3ccc(=O)n(-c4c(F)cccc4F)n3)ccc3nn(C(C)(C)C)cc23)C[C@H]1N] | HCl Salt | LC-MS m/z: 536.3 (M + 1)⁺ ¹H NMR (500 MHz, CD₃OD) δ 8.61-8.55 (m, 1H), 8.25-8.19 (m, 1H), 7.79-7.72 (m, 1H), 7.71-7.61 (m, 1H), 7.34-7.23 (m, 4H), 4.06-4.00 (m, 1H), 3.62-3.57 (m, 1H), 3.48-3.42 (m, 2H), 3.36-3.33 (m, 1H), 1.80-1.76 (m, 9H), 1.20-1.11 (m, 6H). |
| | (S)-N-(4-(4-amino-3,3- | | |
| | dimethylpyrrolidin-1-yl)-2- | | |
| | (tert-butyl)-2H-indazol-5-yl)- | | |
| | 1-(2,6-difluorophenyl)-6- | | |
| | oxo-1,6-dihydropyridazine- | | |
| | 3-carboxamide | | |
| Example 238 | 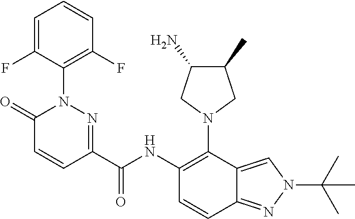 [SMILES: C[C@H]1CN(c2c(NC(=O)c3ccc(=O)n(-c4c(F)cccc4F)n3)ccc3nn(C(C)(C)C)cc23)C[C@@H]1N] | HCOOH Salt | LC-MS m/z: 522.3 (M + 1)+ 1H NMR (400 MHz, CD₃OD) δ 8.44 (s, 1H), 8.24 (d, J = 9.8 Hz, 1H), 7.89 (d, J = 9.2 Hz, 1H), 7.68 (ddd, J = 14.7, 8.5, 6.2 Hz, 1H), 7.40 (d, J = 9.2 Hz, 1H), 7.33 (dd, J = 9.4, 6.5 Hz, 3H), 3.83 (dd, J = 9.7, 7.0 Hz, 2H), 3.42 (dd, J = 10.1, 5.7 Hz, 1H), 3.29 (d, J = 5.7 Hz, 1H), 3.03 (dd, J = 9.3, 4.7 Hz, 1H), 2.38-2.25 (m, 1H), 1.76 (s, 9H), 1.06 (d, J = 6.9 Hz, 3H). |
| | N-(4-((3R,4S)-3-amino-4- | | |
| | methylpyrrolidin-1-yl)-2- | | |
| | (tert-butyl)-2H-indazol-5-yl)- | | |
| | 1-(2,6-difluorophenyl)-6- | | |
| | oxo-1,6-dihydropyridazine- | | |
| | 3-carboxamide | | |
| Example 239 | 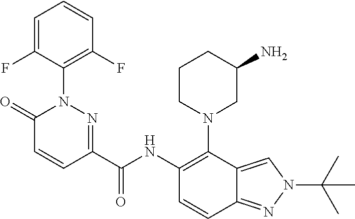 [SMILES: CC(C)(C)n1cc2c(N3CCC[C@@H](N)C3)c(NC(=O)c3ccc(=O)n(-c4c(F)cccc4F)n3)ccc2n1] | HCl Salt | LC-MS m/z: 522.2 (M + 1)+ ¹H NMR (400 MHz, CD₃OD) δ 8.51 (s, 1H), 8.44 (br s, 1H), 8.26 (d, J = 9.9 Hz, 1H), 7.80-7.70 (m, 1H), 7.57 (d, J = 9.3 Hz, 1H), 7.45-7.32 (m, 3H), 3.74 (d, J = 11.1 Hz, 1H), 3.27 (br d, J = 8.2 Hz, 2H), 3.24-3.14 (m, 1H), 3.09 (br s, 1H), 2.97 (br d, J = 11.3 Hz, 1H), 1.96-1.86 (m, 1H), 1.85-1.80 (m, 1H), 1.79 (s, 9H), 1.67- 1.53 (m, 1H), 1.39 (br d, J = 11.2 Hz, 1H). |
| | (R)-N-(4-(3-aminopiperidin- | | |
| | 1-yl)-2-(tert-butyl)-2H- | | |
| | indazol-5-yl)-1-(2,6- | | |
| | difluorophenyl)-6-oxo-1,6- | | |
| | dihydropyridazine-3- | | |
| | carboxamide | | |
| Example 240 | 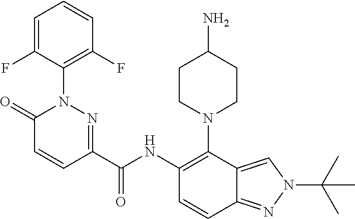 [SMILES: CC(C)(C)n1cc2c(N3CCC(N)CC3)c(NC(=O)c3ccc(=O)n(-c4c(F)cccc4F)n3)ccc2n1] | HCl Salt | LC-MS m/z: 522.2 (M + 1)+ ¹H NMR (400 MHz, CD₃OD) δ 8.59 (s, 1H), 8.25 (d, J = 9.9 Hz, 1H), 8.15 (br d, J = 7.5 Hz, 1H), 7.78-7.66 (m, 1H), 7.51 (d, J = 9.2 Hz, 1H), 7.41-7.26 (m, 3H), 3.37 (br s, 2H), 3.30-3.26 (m, 1H), 3.17 (br s, 2H), 1.94 (br s, 2H), 1.78 (s, 9H), 1.75-1.62 (m, 2H). |
| | N-(4-(4-aminopiperidin-1- | | |
| | yl)-2-(tert-butyl)-2H- | | |
| | indazol-5-yl)-1-(2,6- | | |
| | difluorophenyl)-6-oxo-1,6- | | |
| | dihydropyridazine-3- | | |
| | carboxamide | | |
| Example 241 | 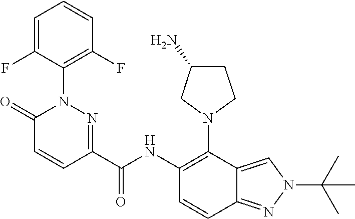 [SMILES: CC(C)(C)n1cc2c(N3CC[C@@H](N)C3)c(NC(=O)c3ccc(=O)n(-c4c(F)cccc4F)n3)ccc2n1] | HCOOH Salt | LC-MS m/z: 508.2 (M + 1)⁺ ¹H NMR (400 MHz, CD₃OD) δ 8.74-8.70 (m, 1H), 8.26-8.18 (m, 1H), 7.78-7.63 (m, 2H), 7.36-7.27 (m, 4H), 3.95-3.81 (m, 3H), 3.63-3.52 (m, 2H), 2.44-2.31 (m, 1H), 2.17-2.04 (m, 1H), 1.83-1.81 (m, 9H). |
| | (R)-N-(4-(3-aminopyrrolidin- | | |
| | 1-yl)-2-(tert-butyl)-2H- | | |
| | indazol-5-yl)-1-(2,6- | | |
| | difluorophenyl)-6-oxo-1,6- | | |
| | dihydropyridazine-3- | | |
| | carboxamide | | |
| Example 242 | 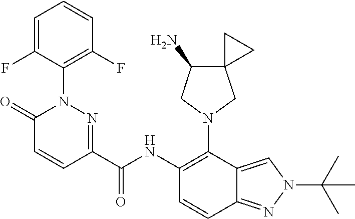 [SMILES: CC(C)(C)n1cc2c(N3C[C@@H](N)C4(CC4)C3)c(NC(=O)c3ccc(=O)n(-c4c(F)cccc4F)n3)ccc2n1] | HCOOH salt | LC-MS m/z: 534.3 (M + 1)⁺ ¹H NMR (400 MHz, CD₃OD) δ 8.47 (s, 1H), 8.21 (d, J = 9.9 Hz, 1H), 7.81 (d, J = 9.2 Hz, 1H), 7.66 (tt, J = 8.7, 6.2 Hz, 1H), 7.36-7.23 (m, 4H), 4.01 (dd, J = 10.0, 5.7 Hz, 1H), 3.82 (d, J = 9.0 Hz, 1H), 3.46 (dd, J = 10.0, 3.3 Hz, 1H), 3.21 (dd, J = 5.8, 3.3 Hz, 1H), 2.99 (d, J = 9.0 Hz, 1H), 1.74 (s, 9H), 0.90 (dt, J = 9.1, 4.9 Hz, 1H), 0.63 (ddt, J = 18.7, 9.4, 4.5 Hz, 2H), 0.53 (ddd, J = 10.5, 5.7, 3.7 Hz, 1H). |
| | (S)-N-(4-(7-amino-5- | | |
| | azaspiro[2.4]heptan-5-yl)-2- | | |
| | (tert-butyl)-2H-indazol-5-yl)- | | |
| | 1-(2,6-difluorophenyl)-6- | | |
| | oxo-1,6-dihydropyridazine- | | |
| | 3-carboxamide | | |
| Example 243 | 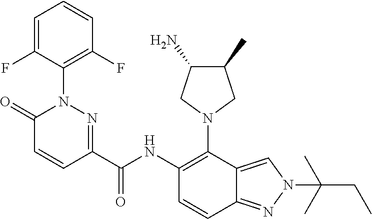 [SMILES: CCC(C)(C)n1cc2c(N3C[C@H](C)[C@@H](N)C3)c(NC(=O)c3ccc(=O)n(-c4c(F)cccc4F)n3)ccc2n1] | HCOOH salt | LC-MS m/z: 536.3 (M + 1)⁺ ¹H NMR (400 MHz, CD₃OD) δ 8.47 (s, 1H), 8.23 (d, J = 9.9 Hz, 1H), 7.89 (d, J = 9.1 Hz, 1H), 7.67 (td, J = 8.9, 4.4 Hz, 1H), 7.39 (d, J = 9.2 Hz, 1H), 7.31 (t, J = 9.0 Hz, 3H), 3.92-3.75 (m, 2H), 3.46 (dd, J = 10.2, 5.8 Hz, 1H), 3.01 (dd, J = 9.6, 4.4 Hz, 1H), 2.42-2.27 (m, 1H), 2.07 (q, J = 7.4 Hz, 2H), 1.73 (s, 6H), 1.04 (d, J = 6.9 Hz, 3H), 0.66 (t, J = 7.3 Hz, 3H). |
| | N-(4-((3R,4S)-3-amino-4- | | |
| | methylpyrrolidin-1-yl)-2- | | |
| | (tert-pentyl)-2H-indazol-5- | | |
| | yl)-1-(2,6-difluorophenyl)-6- | | |
| | oxo-1,6-dihydropyridazine- | | |
| | 3-carboxamide | | |
| Example 244 | 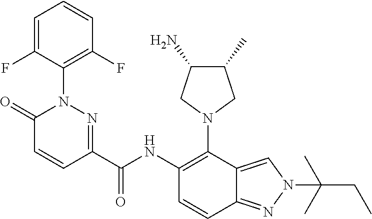 [SMILES: CCC(C)(C)n1cc2c(N3C[C@@H](C)[C@@H](N)C3)c(NC(=O)c3ccc(=O)n(-c4c(F)cccc4F)n3)ccc2n1] | HCOOH Salt | LC-MS m/z: 536.3 (M + 1)⁺ ¹H NMR (400 MHz, CD₃OD) δ 8.42 (s, 1H), 8.20 (d, J = 9.9 Hz, 1H), 7.67 (tt, J = 8.5, 6.2 Hz, 1H), 7.61 (d, J = 9.1 Hz, 1H), 7.30 (td, J = 9.0, 8.2, 4.1 Hz, 4H), 3.96 (dd, J = 10.5, 6.1 Hz, 1H), 3.77 (td, J = 6.3, 3.7 Hz, 1H), 3.55 (dd, J = 9.2, 7.5 Hz, 1H), 3.48 (dd, J = 10.5, 3.7 Hz, 1H), 3.38 (t, J = 8.8 Hz, 1H), 2.53 (dq, J = 14.6, 7.3 Hz, 1H), 2.07 (q, J = 7.4 Hz, 2H), 1.72 (s, 6H), 1.13 (d, J = 7.1 Hz, 3H), 0.66 (t, J = 7.4 Hz, 3H). |
| | 1-(2,6-difluorophenyl)-N-(4- | | |
| | [(3R,4R)-3-amino-4- | | |
| | methyltetrahydro-1H- | | |
| | pyrrol-1-yl]-2-(2-methylbut- | | |
| | 2-yl)indazol-5-yl)-6-oxo-1,2- | | |
| | diazine-3-carboxamide | | |
| Example 245 | 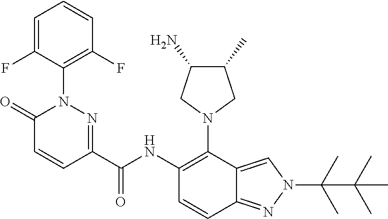 [SMILES: C[C@@H]1CN(c2c(NC(=O)c3ccc(=O)n(-c4c(F)cccc4F)n3)ccc3nn(C(C)(C)C(C)(C)C)cc23)C[C@@H]1N] | HCl Salt | LC-MS m/z: 564.3 (M + 1)⁺ 1H NMR (400 MHz, CD₃OD) δ 8.44 (s, 1H), 8.23 (d, J = 9.9 Hz, 1H), 7.76-7.63 (m, 1H), 7.60-7.53 (m, 1H), 7.37-7.27 (m, 4H), 4.01 (dd, J = 6.1, 10.5 Hz, 1H), 3.87-3.79 (m, 1H), 3.61 (dd, J = 7.6, 9.2 Hz, 1H), 3.52 (dd, J = 3.7, 10.5 Hz, 1H), 3.40 (t, J = 8.8 Hz, 1H), 2.60 (td, J = 7.2, 14.6 Hz, 1H), 1.82 (s, 6H), 1.16 (d, J = 7.2 Hz, 3H), 0.97 (s, 9H). |
| | N-(4-((3R,4R)-3-amino-4- | | |
| | methylpyrrolidin-1-yl)-2- | | |
| | (2,3,3-trimethylbutan-2-yl)- | | |
| | 2H-indazol-5-yl)-1-(2,6- | | |
| | difluorophenyl)-6-oxo-1,6- | | |
| | dihydropyridazine-3- | | |
| | carboxamide | | |
| Example 246 | 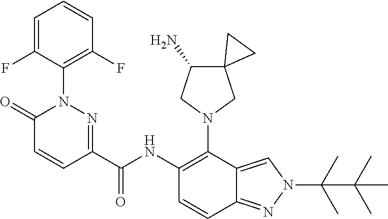 [SMILES: CC(C)(C)C(C)(C)n1cc2c(N3C[C@H](N)C4(CC4)C3)c(NC(=O)c3ccc(=O)n(-c4c(F)cccc4F)n3)ccc2n1] | HCl Salt | LC-MS m/z: 576.3 (M + 1)⁺ 1H NMR (400 MHz, CD₃OD) δ 8.51 (s, 1H), 8.24 (d, J = 9.9 Hz, 1H), 7.74-7.62 (m, 2H), 7.39-7.27 (m, 4H), 4.18 (dd, J = 5.7, 10.5 Hz, 1H), 3.99 (d, J = 9.1 Hz, 1H), 3.64 (dd, J = 2.6, 10.6 Hz, 1H), 3.39 (dd, J = 2.5, 5.5 Hz, 1H), 3.07 (d, J = 9.2 Hz, 1H), 1.83 (s, 6H), 1.07-1.00 (m, 1H), 0.98 (s, 9H), 0.89-0.80 (m, 1H), 0.78-0.63 (m, 2H). |
| | (R)-N-(4-(7-amino-5- | | |
| | azaspiro[2.4]heptan-5-yl)-2- | | |
| | (2,3,3-trimethylbutan-2-yl)- | | |
| | 2H-indazol-5-yl)-1-(2,6- | | |
| | difluorophenyl)-6-oxo-1,6- | | |
| | dihydropyridazine-3- | | |
| | carboxamide | | |
| Example 247 | 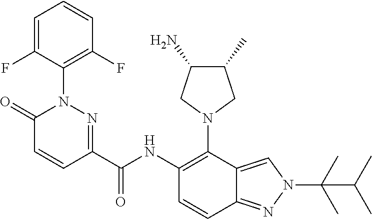 [SMILES: CC(C)C(C)(C)n1cc2c(N3C[C@@H](C)[C@@H](N)C3)c(NC(=O)c3ccc(=O)n(-c4c(F)cccc4F)n3)ccc2n1] | HCl Salt | LC-MS m/z: 550.2 (M + 1)⁺ 1H NMR (400 MHz, CD₃OD) δ 8.62 (s, 1H), 8.22 (d, J = 9.8 Hz, 1H), 7.73-7.64 (m, 1H), 7.63-7.58 (m, 1H), 7.37-7.23 (m, 4H), 4.06 (dd, J = 5.9, 10.7 Hz, 1H), 3.88-3.82 (m, 1H), 3.73-3.64 (m, 1H), 3.60-3.49 (m, 2H), 2.69-2.58 (m, 1H), 2.52 (td, J = 6.7, 13.7 Hz, 1H), 1.76 (s, 6H), 1.19 (d, J = 7.2 Hz, 3H), 0.84 (d, J = 6.8 Hz, 6H). |
| | N-(4-((3R,4R)-3-amino-4- | | |
| | methylpyrrolidin-1-yl)-2- | | |
| | (2,3-dimethylbutan-2-yl)- | | |
| | 2H-indazol-5-yl)-1-(2,6- | | |
| | difluorophenyl)-6-oxo-1,6- | | |
| | dihydropyridazine-3- | | |
| | carboxamide | | |
| Example 248 | 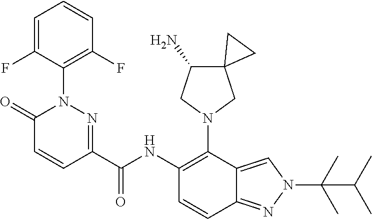 [SMILES: CC(C)C(C)(C)n1cc2c(N3C[C@H](N)C4(CC4)C3)c(NC(=O)c3ccc(=O)n(-c4c(F)cccc4F)n3)ccc2n1] | HCl Salt | LC-MS m/z: 562.3 (M + 1)⁺ 1H NMR (400 MHz, CD₃OD) δ 8.57 (s, 1H), 8.24 (d, J = 9.8 Hz, 1H), 7.74-7.61 (m, 2H), 7.37-7.27 (m, 4H), 4.22 (dd, J = 5.5, 10.7 Hz, 1H), 4.04 (d, J = 9.2 Hz, 1H), 3.66 (dd, J = 2.4, 10.7 Hz, 1H), 3.44- 3.37 (m, 1H), 3.11 (d, J = 9.2 Hz, 1H), 2.52 (quin, J = 6.8 Hz, 1H), 1.74 (s, 6H), 1.09- 0.99 (m, 1H), 0.92-0.85 (m, 1H), 0.83 (d, J = 6.9 Hz, 6H), 0.78-0.66 (m, 2H). |
| | (R)-N-(4-(7-amino-5- | | |
| | azaspiro[2.4]heptan-5-yl)-2- | | |
| | (2,3-dimethylbutan-2-yl)- | | |
| | 2H-indazol-5-yl)-1-(2,6- | | |
| | difluorophenyl)-6-oxo-1,6- | | |
| | dihydropyridazine-3- | | |
| | carboxamide | | |

### Example 249: 1-(2,6-difluorophenyl)-N-{4-[(7R)-7-amino-5-azaspiro[2.4]heptan-5-yl]-1-[(3S)-tetrahydro-3-furyl]indazol-5-yl}-6-oxo-1,2-diazine-3-carboxamide

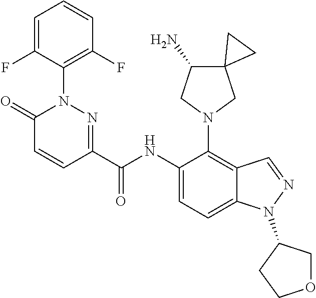 [SMILES: N[C@H]1CN(c2c(NC(=O)c3ccc(=O)n(-c4c(F)cccc4F)n3)ccc3c2cnn3[C@H]2CCOC2)CC12CC2]

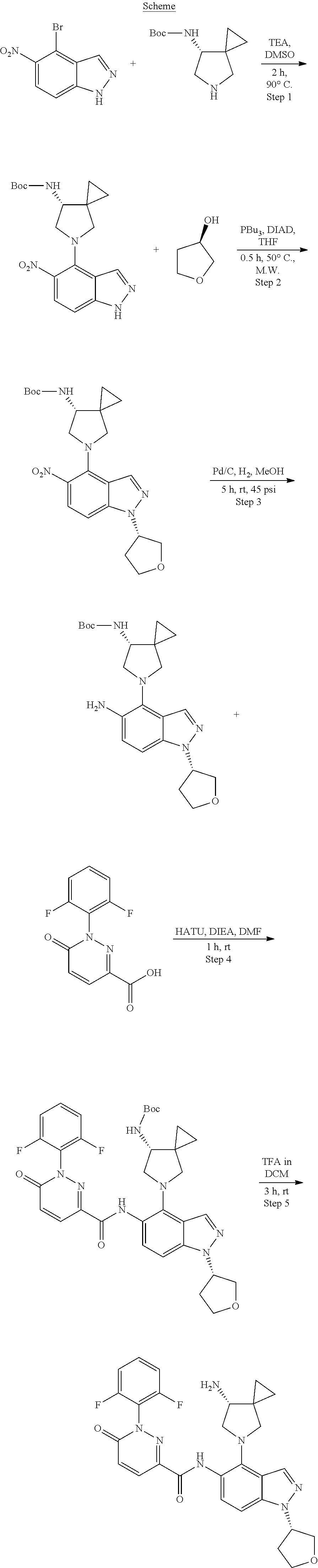

### Step 1: 2-methylpropan-2-yl {[(7R)-5-(5-nitro-1H-indazol-4-yl)-5-azaspiro[2.4]heptan-7-yl]amino}methanoate

To a solution of 4-bromo-5-nitro-1H-indazole (1 g, 4.132 mmol) and 2-methylpropan-2-yl {[(7R)-5-azaspiro[2.4]heptan-7-yl]amino}methanoate (0.92 g, 4.338 mmol) in DMSO (10 mL) was added TEA (1.723 mL, 12.395 mmol). The reaction was stirred for 2 hours at 90° C. to give a brown solution. LCMS showed the starting material was consumed up and one major peak with desired mass was detected. The reaction poured into water and lot of solid was precipitated, filtered and the filter cake was dried by oil pump to afford 2-methylpropan-2-yl{[(7R)-5-(5-nitro-1H-indazol-4-yl)-5-azaspiro[2.4]heptan-7-yl]amino}methanoate (1.47 g, Yield: 95.28%) as a yellow solid.

LC-MS m/z: 374.2 (M+1)⁺

### Step 2: 2-methylpropan-2-yl {[(7R)-5-{5-nitro-1-[(3S)-tetrahydro-3-furyl]indazol-4-yl}-5-azaspiro[2.4]heptan-7-yl]amino}methanoate

To a solution of 2-methylpropan-2-yl{[(7R)-5-(5-nitro-1H-indazol-4-yl)-5-azaspiro[2.4]heptan-7-yl]amino}methanoate (150 mg, 0.402 mmol) and (3R)-tetrahydrofuran-3-ol (0.065 mL, 0.803 mmol) in THF (5 mL) was added tributylphosphane (162.55 mg, 0.803 mmol) and DIAD (162.46 mg, 0.803 mmol). The reaction was stirred for 30 mins by microwave at 50° C. to give a brown solution. LCMS showed most of the starting material was consumed and two new peaks with the desired mass was detected. The reaction was diluted with H₂O and extracted with EA (10 mL×3). The combined organic layers were washed with brine, dried over Na₂SO₄, filtered and concentrated to dryness. The residue was purified by flash column (SiO₂, eluted with 30% EA in PE) to afford 2-methylpropan-2-yl {[(7R)-5-{5-nitro-1-[(3S)-tetrahydro-3-furyl]indazol-4-yl}-5-azaspiro [2.4]heptan-7-yl]amino}methanoate (98 mg, Yield: 55.01%) as a yellow solid.

LC-MS m/z: 444.3 (M+1)⁺

### Step 3: 2-methylpropan-2-yl{[(7R)-5-{5-amino-1-[(3S)-tetrahydro-3-furyl]indazol-4-yl}-5-azaspiro[2.4]heptan-7-yl]amino}methanoate

To a solution of 2-methylpropan-2-yl {[(7R)-5-{5-nitro-1-[(3S)-tetrahydro-3-furyl]indazol-4-yl}-5-azaspiro[2.4]heptan-7-yl]amino}methanoate (98 mg, 0.221 mmol) in MeOH (3 mL) was added Pd/C 10% (20 mg, 0.221 mmol) under N₂. The resulting mixture was degassed with H2 several times and stirred for 5 hours at rt under 45 psi to give a black suspension. LCMS showed most of the starting material was consumed and one major peak with the desired mass was detected. The reaction was filtered through a pad of Celite and the filtrate was concentrated to afford 2-methylpropan-2-yl{[(7R)-5-{5-amino-1-[(3S)-tetrahydro-3-furyl]indazol-4-yl}-5-azaspiro [2.4]heptan-7-yl]amino}methanoate (95 mg, Yield: 103.97%) as a brown solid.

LC-MS m/z: 414.3 (M+1)⁺

### Step 4: 2-methylpropan-2-yl {[(7R)-5-[5-({[1-(2,6-difluorophenyl)-6-oxo-1,2-diazin-3-yl]carbonyl}amino)-1-[(3S)-tetrahydro-3-furyl]indazol-4-yl]-5-azaspiro[2.4]heptan-7-yl]amino}methanoate

To a solution of 1-(2,6-difluorophenyl)-6-oxo-1,2-diazine-3-carboxylic acid (50 mg, 0.198 mmol) and 2-methylpropan-2-yl{[(7R)-5-{5-amino-1-[(3S)-tetrahydro-3-furyl]indazol-4-yl}-5-azaspiro[2.4]heptan-7-yl]amino}methanoate (90.19 mg, 0.218 mmol) in DMF (1 mL) was added DIEA (0.098 mL, 0.595 mmol) and HATU (113.09 mg, 0.297 mmol). The residue was stirred for 1 hour at rt to give a brown solution. LCMS showed the starting material was consumed up and one major peak with desired mass was detected. The residue was poured into water and a lot of solid was precipitated, filtered and dried to afford 2-methylpropan-2-yl{[(7R)-5-[5-({[1-(2,6-difluorophenyl)-6-oxo-1,2-diazin-3-yl]carbonyl}amino)-1-[(3S)-tetrahydro-3-furyl]indazol-4-yl]-5-azaspiro[2.4]heptan-7-yl]amino}methanoate (115 mg, Yield: 89.55%) as a brown solid.

LC-MS m/z: 648.3 (M+1)⁺

### Step 5: 1-(2,6-difluorophenyl)-N-{4-[(7R)-7-amino-5-azaspiro[2.4]heptan-5-yl]-1-[(3S)-tetrahydro-3-furyl]indazol-5-yl}-6-oxo-1,2-diazine-3-carboxamide

To a solution of 2-methylpropan-2-yl{[(7R)-5-[5-({[1-(2,6-difluorophenyl)-6-oxo-1,2-diazin-3-yl]carbonyl}amino)-1-[(3S)-tetrahydro-3-furyl]indazol-4-yl]-5-azaspiro[2.4]heptan-7-yl]amino}methanoate (115 mg, 0.178 mmol) in DCM (6 mL) was added 50% TFA in DCM (2 mL) dropwise. The reaction mixture was stirred for 3 hours at rt to give a brown solution. LCMS showed the starting material was consumed up and one major peak with desired mass was detected. The reaction was concentrated to dryness. The residue was basified with NH₄OH to pH=7^{˜}8 and then purified by flash column five times (C₁₈, eluted with a gradient of ACN/H₂O containing 0.5% HCOOH, at a rate of 45 mL/min) and lyophilized to afford Example 249 (36.4 mg, Yield: 37.44%) as a yellow solid.

LC-MS m/z: 548.2 (M+1)⁺

¹H NMR (400 MHz, CD₃OD): δ 8.30-8.19 (m, 2H), 7.75 (d, J=9.0 Hz, 1H), 7.66 (tt, J=8.6, 6.3 Hz, 1H), 7.29 (ddd, J=10.7, 8.7, 3.5 Hz, 4H), 5.40 (tt, J=8.0, 3.9 Hz, 1H), 4.24-4.10 (m, 3H), 4.05 (dd, J=9.3, 3.8 Hz, 1H), 3.96 (td, J=8.5, 6.0 Hz, 2H), 3.57 (dd, J=10.2, 3.0 Hz, 1H), 3.25 (dd, J=5.5, 2.9 Hz, 1H), 3.12 (d, J=9.2 Hz, 1H), 2.51 (dq, J=12.8, 7.8 Hz, 1H), 2.46-2.34 (m, 1H), 0.92 (dt, J=8.7, 4.5 Hz, 1H), 0.67 (tdd, J=15.5, 7.0, 2.6 Hz, 3H).

| No. | Structure | Salt form | MS and ¹HNMR |
|---|---|---|---|
| Example 250 | 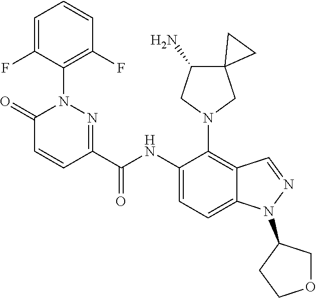 [SMILES: N[C@H]1CN(c2c(NC(=O)c3ccc(=O)n(-c4c(F)cccc4F)n3)ccc3c2cnn3[C@@H]2CCOC2)CC12CC2] | HCOOH salt | LC-MS m/z: 548.2 (M + 1)⁺ ¹H NMR (400 MHz, CD₃OD) δ 8.28- 8.18 (m, 2H), 7.82 (d, J = 8.9 Hz, 1H), 7.66 (tt, J = 8.5, 6.2 Hz, 1H), 7.29 (td, J = 9.6, 4.1 Hz, 4H), 5.40 (tt, J = 7.7, 4.0 Hz, 1H), 4.24-4.11 (m, 2H), 4.13- 4.03 (m, 2H), 3.96 (td, J = 8.1, 5.9 Hz, 1H), 3.88 (d, J = 9.1 Hz, 1H), 3.51 (dd, J = 9.8, 3.4 Hz, 1H), 3.18 (dd, J = 5.5, 3.3 Hz, 1H), 3.12 (d, J = 9.1 Hz, 1H), 2.51 (dq, J = 15.2, 7.7 Hz, 1H), 2.45- 2.33 (m, 1H), 0.92-0.84 (m, 1H), 0.67-0.56 (m, 3H). |
| | N-(4-((R)-7-amino-5- | | |
| | azaspiro[2.4]heptan-5-yl)-1- | | |
| | ((R)-tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 251 | 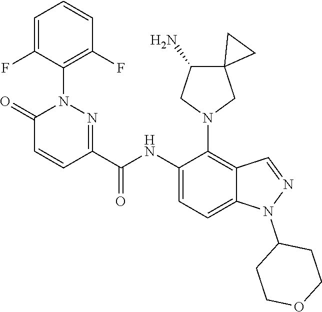 [SMILES: N[C@H]1CN(c2c(NC(=O)c3ccc(=O)n(-c4c(F)cccc4F)n3)ccc3c2cnn3C2CCOCC2)CC12CC2] | HCOOH salt | LC-MS m/z: 562.3 (M + 1)⁺ ¹H NMR (400 MHz, CD₃OD) δ 8.26 (d, J = 0.8 Hz, 1H), 8.21 (d, J = 9.9 Hz, 1H), 7.72-7.61 (m, 2H), 7.35-7.24 (m, 4H), 4.78 (dt, J = 11.5, 4.2 Hz, 1H), 4.22 (dd, J = 10.7, 5.6 Hz, 1H), 4.10 (dd, J = 12.3, 4.1 Hz, 2H), 4.03 (d, J = 9.3 Hz, 1H), 3.73-3.62 (m, 3H), 3.38 (dd, J = 5.6, 2.5 Hz, 1H), 3.11 (d, J = 9.3 Hz, 1H), 2.27 (qdd, J = 11.9, 6.7, 4.6 Hz, 2H), 1.92 (d, J = 12.6 Hz, 2H), 0.99 (dt, J = 9.9, 5.7 Hz, 1H), 0.81 (dt, J = 9.2, 5.9 Hz, 1H), 0.70 (dddd, J = 19.0, 10.6, 6.2, 4.3 Hz, 2H). |
| | (R)-N-(4-(7-amino-5- | | |
| | azaspiro[2.4]heptan-5-yl)-1- | | |
| | (tetrahydro-2H-pyran-4-yl)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 252 | 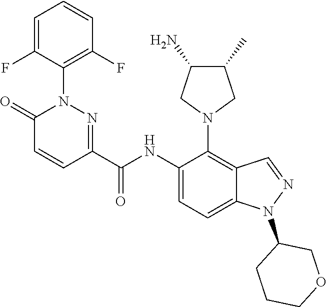 [SMILES: C[C@@H]1CN(c2c(NC(=O)c3ccc(=O)n(-c4c(F)cccc4F)n3)ccc3c2cnn3[C@@H]2CCCOC2)C[C@@H]1N] | HCOOH salt | LC-MS m/z: 550.3 (M + 1)⁺ ¹H NMR (400 MHz, CD₃OD) δ 8.23 (s, 1H), 8.19 (d, J = 9.9 Hz, 1H), 7.70- 7.58 (m, 2H), 7.32-7.23 (m, 3H), 7.20 (d, J = 8.9 Hz, 1H), 4.64 (tt, J = 10.7, 4.4 Hz, 1H), 4.07-4.00 (m, 1H), 4.00-3.92 (m, 2H), 3.81-3.71 (m, 2H), 3.63 (dd, J = 9.3, 7.5 Hz, 1H), 3.58- 3.47 (m, 2H), 3.44 (t, J = 8.8 Hz, 1H), 2.54 (hept, J = 7.2 Hz, 1H), 2.26 (dtd, J = 12.8, 11.0, 6.2 Hz, 1H), 2.20-2.10 (m, 1H), 1.97-1.81 (m, 2H), 1.11 (d, J = 7.1 Hz, 3H). |
| | N-(4-((3R,4R)-3-amino-4- | | |
| | methylpyrrolidin-1-yl)-1-((R)- | | |
| | tetrahydro-2H-pyran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 253 | 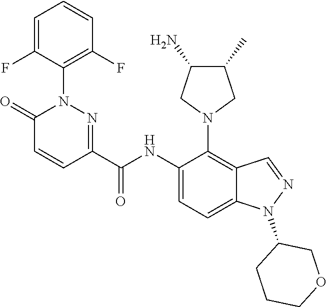 [SMILES: C[C@@H]1CN(c2c(NC(=O)c3ccc(=O)n(-c4c(F)cccc4F)n3)ccc3c2cnn3[C@H]2CCCOC2)C[C@@H]1N] | HCOOH salt | LC-MS m/z: 550.3 (M + 1)⁺ ¹H NMR (400 MHz, CD₃OD) δ 8.23 (s, 1H), 8.20 (d, J = 9.9 Hz, 1H), 7.65 (tt, J = 8.6, 6.2 Hz, 1H), 7.60 (d, J = 8.9 Hz, 1H), 7.34-7.25 (m, 3H), 7.22 (d, J = 8.9 Hz, 1H), 4.65 (tt, J = 10.2, 4.3 Hz, 1H), 4.07 (ddd, J = 10.7, 6.0, 1.8 Hz, 1H), 4.02-3.92 (m, 2H), 3.81 (td, J = 6.2, 3.6 Hz, 1H), 3.75 (t, J = 10.5 Hz, 1H), 3.66 (dd, J = 9.4, 7.6 Hz, 1H), 3.57 (dd, J = 10.7, 3.5 Hz, 1H), 3.48 (ddd, J = 17.9, 11.1, 6.1 Hz, 2H), 2.59 (hept, J = 7.2 Hz, 1H), 2.28 (ddd, J = 16.8, 12.6, 9.0 Hz, 1H), 2.16 (d, J = 12.7 Hz, 1H), 1.89 (ddt, J = 9.7, 7.6, 3.8 Hz, 2H), 1.13 (d, J = 7.1 Hz, 3H). |
| | N-(4-((3R,4R)-3-amino-4- | | |
| | methylpyrrolidin-1-yl)-1-((S)- | | |
| | tetrahydro-2H-pyran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 254 | 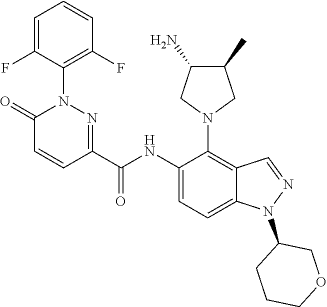 [SMILES: C[C@H]1CN(c2c(NC(=O)c3ccc(=O)n(-c4c(F)cccc4F)n3)ccc3c2cnn3[C@@H]2CCCOC2)C[C@@H]1N] | HCOOH salt | LC-MS m/z: 550.3 (M + 1)⁺ ¹H NMR (400 MHz, CD₃OD) δ 8.28- 8.17 (m, 2H), 7.86 (d, J = 9.0 Hz, 1H), 7.66 (tt, J = 8.6, 6.2 Hz, 1H), 7.29 (ddd, J = 10.5, 8.7, 5.4 Hz, 4H), 4.67 (td, J = 10.4, 5.0 Hz, 1H), 3.99 (ddt, J = 10.8, 6.6, 3.3 Hz, 2H), 3.92-3.81 (m, 2H), 3.77 (t, J = 10.5 Hz, 1H), 3.60- 3.40 (m, 2H), 3.29-3.24 (m, 1H), 3.18-3.06 (m, 1H), 2.26 (tt, J = 10.9, 4.2 Hz, 2H), 2.18 (t, J = 7.4 Hz, 1H), 1.96-1.82 (m, 2H), 1.06 (d, J = 6.9 Hz, 3H). |
| | N-(4-((3R,4S)-3-amino-4- | | |
| | methylpyrrolidin-1-yl)-1-((R)- | | |
| | tetrahydro-2H-pyran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 255 | 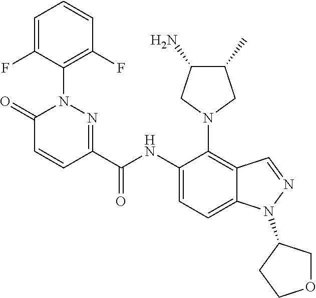 [SMILES: C[C@@H]1CN(c2c(NC(=O)c3ccc(=O)n(-c4c(F)cccc4F)n3)ccc3c2cnn3[C@H]2CCOC2)C[C@@H]1N] | HCOOH salt | LC-MS m/z: 536.6 (M + 1)⁺ ¹H NMR (400 MHz, CD₃OD) δ 8.28- 8.18 (m, 2H), 7.72-7.61 (m, 2H), 7.31 (dd, J = 9.4, 5.6 Hz, 3H), 7.21 (d, J = 8.9 Hz, 1H), 5.40 (tt, J = 7.8, 4.0 Hz, 1H), 4.27-4.11 (m, 2H), 4.10- 3.93 (m, 3H), 3.76-3.59 (m, 2H), 3.57-3.42 (m, 2H), 2.59-2.35 (m, 3H), 1.11 (d, J = 7.1 Hz, 3H). |
| | N-(4-((3R,4R)-3-amino-4- | | |
| | methylpyrrolidin-1-yl)-1-((S)- | | |
| | tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 256 | 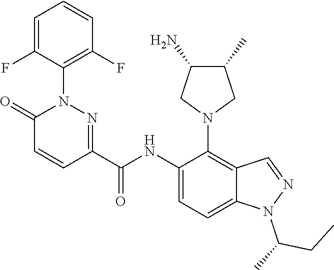 [SMILES: CC[C@H](C)n1ncc2c(N3C[C@@H](C)[C@@H](N)C3)c(NC(=O)c3ccc(=O)n(-c4c(F)cccc4F)n3)ccc21] | HCl salt | LC-MS m/z: 522.2 (M + 1)⁺ ¹H NMR (400 MHz, CD₃OD) δ 8.30 (s, 1H), 8.23 (d, J = 9.8 Hz, 1H), 7.68 (tt, J = 6.3, 8.6 Hz, 1H), 7.63-7.56 (m, 1H), 7.37-7.18 (m, 4H), 4.72-4.61 (m, 1H), 4.11 (dd, J = 6.0, 10.8 Hz, 1H), 3.89-3.82 (m, 1H), 3.71 (t, J = 8.5 Hz, 1H), 3.62 (dd, J = 3.4, 10.7 Hz, 1H), 3.50 (t, J = 8.9 Hz, 1H), 2.63 (td, J = 7.3, 14.4 Hz, 1H), 2.13-2.00 (m, 1H), 1.97- 1.85 (m, 1H), 1.53 (d, J = 6.6 Hz, 3H), 1.16 (d, J = 7.1 Hz, 3H), 0.74 (t, J = 7.4 Hz, 3H). |
| | N-(4-((3R,4R)-3-amino-4- | | |
| | methylpyrrolidin-1-yl)-1-((S)- | | |
| | sec-butyl)-1H-indazol-5-yl)-1- | | |
| | (2,6-difluorophenyl)-6-oxo- | | |
| | 1,6-dihydropyridazine-3- | | |
| | carboxamide | | |
| Example 257 | 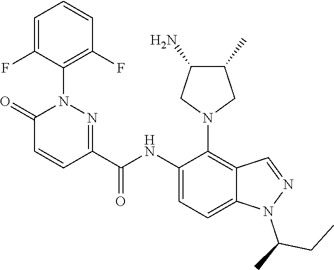 [SMILES: CC[C@@H](C)n1ncc2c(N3C[C@@H](C)[C@@H](N)C3)c(NC(=O)c3ccc(=O)n(-c4c(F)cccc4F)n3)ccc21] | HCl salt | LC-MS m/z: 522.2 (M + 1)⁺ 1H NMR (400 MHz, CD₃OD) δ 8.26 (s, 1H), 8.20 (d, J = 9.8 Hz, 1H), 8.29-8.16 (m, 1H), 7.70-7.63 (m, 1H), 7.56 (br d, J = 8.9 Hz, 1H), 7.34-7.26 (m, 2H), 7.33-7.17 (m, 1H), 4.66 (br d, J = 5.6 Hz, 1H), 4.09 (br dd, J = 6.0, 10.7 Hz, 1H), 3.82 (br d, J = 3.6 Hz, 1H), 3.72- 3.66 (m, 1H), 3.58 (br dd, J = 3.2, 10.7 Hz, 1H), 3.48 (br t, J = 8.9 Hz, 1H), 2.66- 2.57 (m, 1H), 2.12-1.98 (m, 1H), 195-1.84 (m, 1H), 1.51 (br d, J = 6.6 Hz, 3H), 1.14 (br d, J = 7.0 Hz, 3H), 0.77-0.69 (m, 3H). |
| | N-(4-((3R,4R)-3-amino-4- | | |
| | methylpyrrolidin-1-yl)-1-((R)- | | |
| | sec-butyl)-1H-indazol-5-yl)-1- | | |
| | (2,6-difluorophenyl)-6-oxo- | | |
| | 1,6-dihydropyridazine-3- | | |
| | carboxamide | | |
| Example 258 | 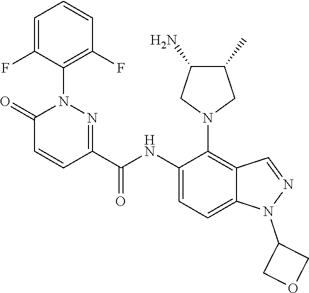 [SMILES: C[C@@H]1CN(c2c(NC(=O)c3ccc(=O)n(-c4c(F)cccc4F)n3)ccc3c2cnn3C2COC2)C[C@@H]1N] | HCOOH salt | LC-MS m/z: 522.5 (M + 1)⁺ ¹H NMR (400 MHz, CD₃OD) δ 8.38 (s, 1H), 8.21 (d, J = 9.9 Hz, 1H), 7.72- 7.59 (m, 2H), 7.35-7.27 (m, 3H), 7.16 (d, J = 8.9 Hz, 1H), 5.93 (p, J = 6.8 Hz, 1H), 5.25-5.10 (m, 4H), 4.04 (ddd, J = 18.7, 11.7, 6.6 Hz, 2H), 3.75 (td, J = 5.9, 3.5 Hz, 1H), 3.67 (dd, J = 9.3, 7.5 Hz, 1H), 3.60-3.44 (m, 2H), 2.56 (dq, J = 14.3, 7.2 Hz, 1H), 1.13 (d, J = 7.1 Hz, 3H). |
| | N-(4-((3R,4R)-3-amino-4- | | |
| | methylpyrrolidin-1-yl)-1- | | |
| | (oxetan-3-yl)-1H-indazol-5-yl)- | | |
| | 1-(2,6-difluorophenyl)-6-oxo- | | |
| | 1,6-dihydropyridazine-3- | | |
| | carboxamide | | |
| Example 259 | 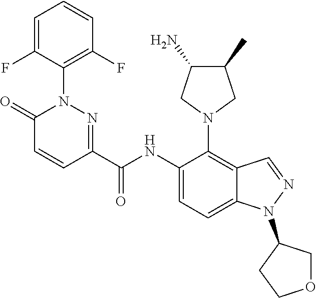 [SMILES: C[C@H]1CN(c2c(NC(=O)c3ccc(=O)n(-c4c(F)cccc4F)n3)ccc3c2cnn3[C@@H]2CCOC2)C[C@@H]1N] | HCl salt | LC-MS m/z: 536.2 (M + 1)⁺ ¹H NMR (500 MHz, CD₃OD) δ 8.18- 8.27 (m, 2H), 7.87 (d, J = 9.00 Hz, 1H), 7.63-7.71 (m, 1H), 7.27-7.39 (m, 4H), 5.39-5.51 (m, 1H), 4.15-4.27 (m, 2H), 4.08 (dd, J = 3.66, 9.46 Hz, 1H), 3.87- 4.03 (m, 3H), 3.52 (dd, J = 5.72, 10.15 Hz, 1H), 3.16 (dd, J = 5.04, 9.31 Hz, 1H), 2.49-2.59 (m, 1H), 2.32-2.46 (m, 2H), 1.10 (d, J = 7.02 Hz, 3H). |
| | N-(4-((3R,4S)-3-amino-4- | | |
| | methylpyrrolidin-1-yl)-1-((R)- | | |
| | tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 260 | 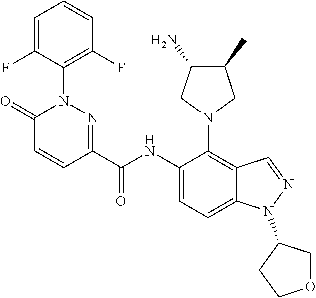 [SMILES: C[C@H]1CN(c2c(NC(=O)c3ccc(=O)n(-c4c(F)cccc4F)n3)ccc3c2cnn3[C@H]2CCOC2)C[C@@H]1N] | HCl salt | LC-MS m/z: 536.2 (M + 1)⁺ ¹H NMR (500 MHz, CD₃OD) δ 8.27 (s, 1H), 8.24 (d, J = 9.9 Hz, 1H), 7.88 (d, J = 9.0 Hz, 1H), 7.74-7.64 (m, 1H), 7.38-7.27 (m, 1H), 7.38-7.27 (m, 3H), 5.47-5.40 (m, 1H), 4.26-4.13 (m, 2H), 4.08 (dd, J = 3.7, 9.5 Hz, 1H), 4.01-3.88 (m, 3H), 3.53 (dd, J = 5.7, 10.1 Hz, 1H), 3.41-3.36 (m, 1H), 3.15 (dd, J = 5.1, 9.4 Hz, 1H), 2.57-2.49 (m, 1H), 2.44-2.31 (m, 2H), 1.09 (d, J = 6.9 Hz, 3H). |
| | N-(4-((3R,4S)-3-amino-4- | | |
| | methylpyrrolidin-1-yl)-1-((S)- | | |
| | tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 261 | 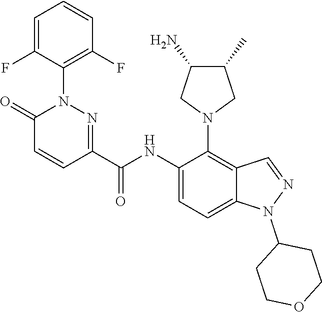 [SMILES: C[C@@H]1CN(c2c(NC(=O)c3ccc(=O)n(-c4c(F)cccc4F)n3)ccc3c2cnn3C2CCOCC2)C[C@@H]1N] | HCl salt | LC-MS m/z: 550.2 (M + 1)⁺ 1H NMR (400 MHz, CD₃OD) δ 8.30- 8.17 (m, 2H), 7.72-7.59 (m, 2H), 7.36-7.23 (m, 4H), 4.81 (br s, 1H), 4.15-4.07 (m, 3H), 3.87-3.81 (m, 1H), 3.72-3.65 (m, 3H), 3.61-3.57 (m, 1H), 3.51-3.46 (m, 1H), 2.66- 2.58 (m, 1H), 2.33-2.23 (m, 2H), 1.99-1.90 (m, 2H), 1.16 (d, J = 7.0 Hz, 3H). |
| | N-(4-((3R,4R)-3-amino-4- | | |
| | methylpyrrolidin-1-yl)-1- | | |
| | (tetrahydro-2H-pyran-4-yl)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 262 | 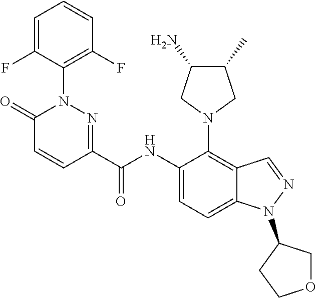 [SMILES: C[C@@H]1CN(c2c(NC(=O)c3ccc(=O)n(-c4c(F)cccc4F)n3)ccc3c2cnn3[C@@H]2CCOC2)C[C@@H]1N] | HCOOH salt | LC-MS m/z: 536.3 (M + 1)⁺ ¹H NMR (400 MHz, CD₃OD) δ 8.30- 8.19 (m, 2H), 7.63 (d, J = 8.9 Hz, 3H), 7.32 (d, J = 10.0 Hz, 3H), 7.22 (d, J = 8.9 Hz, 1H), 5.41 (d, J = 8.5 Hz, 2H), 4.25-4.12 (m, 3H), 4.11-3.94 (m, 4H), 3.76-3.61 (m, 3H), 3.55-3.41 (m, 3H), 2.52 (dd, J = 13.5, 7.0 Hz, 3H), 2.41 (d, J = 5.1 Hz, 2H), 1.11 (d, J = 7.0 Hz, 3H). |
| | N-(4-((3R,4R)-3-amino-4- | | |
| | methylpyrrolidin-1-yl)-1-((R)- | | |
| | tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 263 | 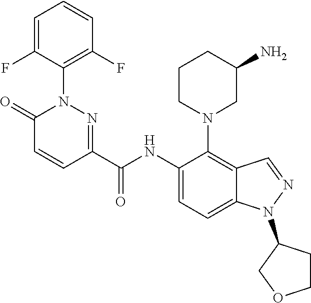 [SMILES: N[C@@H]1CCCN(c2c(NC(=O)c3ccc(=O)n(-c4c(F)cccc4F)n3)ccc3c2cnn3[C@H]2CCOC2)C1] | HCOOH salt | LC-MS m/z: 536.3 (M + 1)⁺ ¹H NMR (400 MHz, CD₃OD) δ 8.55 (d, J = 9.2 Hz, 1H), 8.26 (d, J = 9.9 Hz, 1H), 8.20 (d, J = 9.5 Hz, 1H), 7.83-7.65 (m, 1H), 7.57 (d, J = 9.1 Hz, 1H), 7.37 (dd, J = 24.1, 9.5 Hz, 3H), 5.46 (dd, J = 12.7, 5.8 Hz, 1H), 4.19 (ddd, J = 13.1, 12.2, 7.1 Hz, 2H), 4.08 (dd, J = 9.4, 3.5 Hz, 1H), 3.99 (dd, J = 14.2, 8.2 Hz, 1H), 3.26 (s, 1H), 3.12 (dd, J = 20.1, 10.3 Hz, 2H), 2.97 (d, J = 11.4 Hz, 1H), 2.90 (s, 1H), 2.54 (td, J = 15.2, 8.1 Hz, 1H), 2.47-2.34 (m, 1H), 1.80 (s, 1H), 1.73 (d, J = 9.2 Hz, 1H), 1.40 (dd, J = 19.4, 10.5 Hz, 2H). |
| | N-(4-((R)-3-aminopiperidin-1- | | |
| | yl)-1-((S)-tetrahydrofuran-3- | | |
| | yl)-1H-indazol-5-yl)-1-(2,6- | | |
| | difluorophenyl)-6-oxo-1,6- | | |
| | dihydropyridazine-3- | | |
| | carboxamide | | |
| Example 264 | 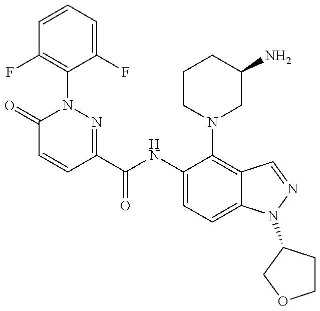 [SMILES: N[C@@H]1CCCN(c2c(NC(=O)c3ccc(=O)n(-c4c(F)cccc4F)n3)ccc3c2cnn3[C@@H]2CCOC2)C1] | HCOOH salt | LC-MS m/z: 536.2 (M + 1)⁺. ¹H NMR (400 MHz, CD₃OD) δ 8.51 (d, J = 8.6 Hz, 1H), 8.40 (s, 1H), 8.27 (d, J = 9.8 Hz, 1H), 8.21 (s, 1H), 7.80-7.66 (m, 1H), 7.58 (d, J = 9.1 Hz, 1H), 7.37 (dd, J = 20.4, 9.5 Hz, 3H), 5.48 (d, J = 7.2 Hz, 1H), 4.29-4.15 (m, 2H), 4.07 (dd, J = 9.4, 3.6 Hz, 1H), 3.99 (dd, J = 14.1, 8.1 Hz, 1H), 3.25 (t, J = 10.3 Hz, 1H), 3.14 (t, J = 10.7 Hz, 1H), 3.02 (dd, J = 14.2, 10.9 Hz, 2H), 2.55 (td, J = 15.3, 8.0 Hz, 1H), 2.42 (dd, J = 17.4, 12.9 Hz, 1H), 1.89 (d, J = 11.1 Hz, 1H), 1.78 (d, J = 13.2 Hz, 1H), 1.54 (dd, J = 22.1, 10.2 Hz, 1H), 1.47-1.34 (m, 1H). |
| | N-(4-((R)-3-aminopiperidin-1- | | |
| | yl)-1-((R)-tetrahydrofuran-3- | | |
| | yl)-1H-indazol-5-yl)-1-(2,6- | | |
| | difluorophenyl)-6-oxo-1,6- | | |
| | dihydropyridazine-3- | | |
| | carboxamide | | |
| Example 265 | 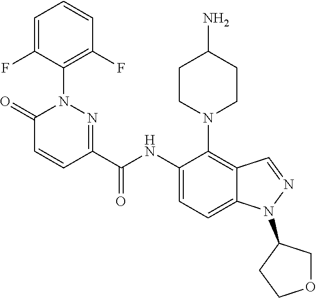 [SMILES: NC1CCN(c2c(NC(=O)c3ccc(=O)n(-c4c(F)cccc4F)n3)ccc3c2cnn3[C@@H]2CCOC2)CC1] | HCOOH salt | LC-MS m/z: 536.2 (M + 1)⁺. ¹H NMR (400 MHz, CD₃OD) δ 8.36- 8.19 (m, 3H), 7.71 (tt, J = 8.5, 6.2 Hz, 1H), 7.50 (dd, J = 9.1, 0.9 Hz, 1H), 7.41-7.27 (m, 3H), 5.45 (dq, J = 10.7, 3.8 Hz, 1H), 4.27-4.13 (m, 2H), 4.08 (dd, J = 9.3, 3.7 Hz, 1H), 3.99 (td, J = 8.2, 5.9 Hz, 1H), 3.20 (ddd, J = 12.0, 8.9, 3.0 Hz, 2H), 3.11 (dt, J = 8.9, 4.6 Hz, 1H), 2.54 (dtd, J = 13.0, 8.2, 6.8 Hz, 1H), 2.41 (dddd, J = 13.2, 7.6, 5.8, 4.0 Hz, 1H), 1.93 (d, J = 11.4 Hz, 2H), 1.56 (d, J = 9.2 Hz, 2H). |
| | (R)-N-(4-(4-aminopiperidin-1- | | |
| | yl)-1-(tetrahydrofuran-3-yl)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 266 | 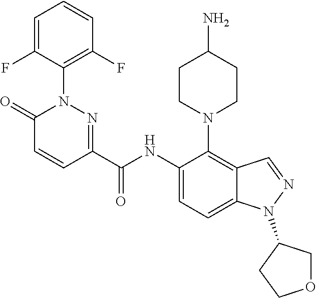 [SMILES: NC1CCN(c2c(NC(=O)c3ccc(=O)n(-c4c(F)cccc4F)n3)ccc3c2cnn3[C@H]2CCOC2)CC1] | HCOOH salt | LC-MS m/z: 536.2 (M + 1)⁺. ¹H NMR (400 MHz, CD₃OD) δ 8.35- 8.17 (m, 3H), 7.71 (tt, J = 8.6, 6.2 Hz, 1H), 7.50 (dd, J = 9.1, 0.9 Hz, 1H), 7.43-7.25 (m, 3H), 5.45 (dq, J = 10.7, 3.9 Hz, 1H), 4.26-4.14 (m, 2H), 4.08 (dd, J = 9.4, 3.7 Hz, 1H), 3.99 (td, J = 8.1, 5.8 Hz, 1H), 3.37 (d, J = 4.6 Hz, 1H), 3.17 (dtd, J = 22.4, 8.8, 3.8 Hz, 3H), 2.54 (dtd, J = 13.0, 8.2, 6.9 Hz, 1H), 2.41 (dddd, J = 13.1, 7.6, 5.9, 4.0 Hz, 1H), 1.94 (dt, J = 14.2, 4.2 Hz, 2H), 1.60 (qd, J = 8.7, 4.3 Hz, 2H). |
| | (S)-N-(4-(4-aminopiperidin-1- | | |
| | yl)-1-(tetrahydrofuran-3-yl)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 267 | 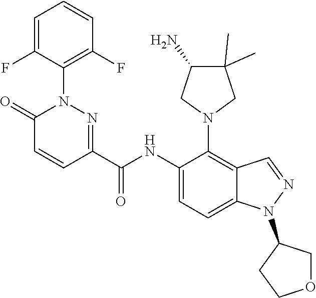 [SMILES: CC1(C)CN(c2c(NC(=O)c3ccc(=O)n(-c4c(F)cccc4F)n3)ccc3c2cnn3[C@@H]2CCOC2)C[C@@H]1N] | Free base | LC-MS m/z: 550.2 (M + 1)⁺. ¹H NMR (400 MHz, CD₃OD) δ 8.19- 8.10 (m, 2H), 7.66-7.61 (m, 1H), 7.55-7.50 (m, 1H), 7.20-7.16 (m, 4H), 5.27-5.20 (m, 1H), 4.07-4.02 (m, 2H), 3.98-3.89 (m, 2H), 3.85- 3.79 (m, 1H), 3.47-3.45 (m, 1H), 3.40-3.30 (m, 2H), 3.29-3.20 (m, 1H), 2.40-2.30 (m, 2H), 1.05-1.02 (m, 6H). |
| | N-(4-((R)-4-amino-3,3- | | |
| | dimethylpyrrolidin-1-yl)-1- | | |
| | ((R)-tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 268 | 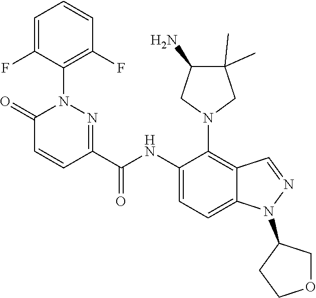 [SMILES: CC1(C)CN(c2c(NC(=O)c3ccc(=O)n(-c4c(F)cccc4F)n3)ccc3c2cnn3[C@@H]2CCOC2)C[C@H]1N] | Free base | LC-MS m/z: 550.2 (M + 1)⁺. ¹H NMR (400 MHz, CD₃OD) δ = 8.23- 8.21 (m, 2H), 7.76-7.71 (m, 1H), 7.65-7.60 (m, 1H), 7.30-7.26 (m, 4H), 5.37-5.30 (m, 1H), 4.17-4.12 (m, 2H), 4.02-3.95 (m, 2H), 3.95- 3.89 (m, 1H), 3.57-3.55 (m, 1H), 3.50-3.40 (m, 2H), 3.39-3.30 (m, 1H), 2.540-2.430 (m, 2H), 1.15- 1.12 (m, 6H). |
| | N-(4-((S)-4-amino-3,3- | | |
| | dimethylpyrrolidin-1-yl)-1- | | |
| | ((R)-tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 269 | 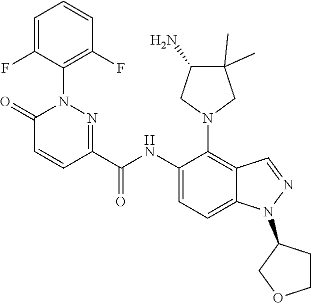 [SMILES: CC1(C)CN(c2c(NC(=O)c3ccc(=O)n(-c4c(F)cccc4F)n3)ccc3c2cnn3[C@H]2CCOC2)C[C@@H]1N] | Free base | LC-MS m/z: 550.2 M + 1)⁺. 1H NMR (400 MHz, CD₃OD) δ 8.30- 8.19 (m, 2H), 7.83-7.73 (m, 1H), 7.73-7.61 (m, 1H), 7.36-7.25 (m, 4H), 5.46-5.38 (m, 1H), 4.25-4.13 (m, 2H), 4.13-4.05 (m, 2H), 4.03- 3.94 (m, 1H), 3.59 (d, J = 9.3 Hz, 1H), 3.54-3.44 (m, 3H), 3.39 (br d, J = 9.3 Hz, 2H), 2.61-2.47 (m, 1H), 2.46- 2.36 (m, 1H), 1.16 (d, J = 10.7 Hz, 6H). |
| | N-(4-((R)-4-amino-3,3- | | |
| | dimethylpyrrolidin-1-yl)-1- | | |
| | ((S)-tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 270 | 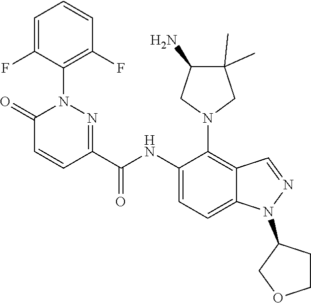 [SMILES: CC1(C)CN(c2c(NC(=O)c3ccc(=O)n(-c4c(F)cccc4F)n3)ccc3c2cnn3[C@H]2CCOC2)C[C@H]1N] | Free base | LC-MS m/z: 550.2 M + 1)⁺. ¹H NMR (400 MHz, CD₃OD) δ 8.20- 8.03 (m, 2H), 7.69-7.62 (m, 1H), 7.56 (tt, J = 6.3, 8.6 Hz, 1H), 7.24-7.15 (m, 4H), 5.34-5.27 (m, 1H), 4.12- 4.01 (m, 2H), 4.00-3.92 (m, 2H), 3.91-3.82 (m, 1H), 3.47 (d, J = 9.3 Hz, 1H), 3.42-3.33 (m, 2H), 3.27 (d, J = 9.3 Hz, 1H), 2.49-2.36 (m, 1H), 2.34-2.23 (m, 1H), 1.04 (d, J = 9.9 Hz, 6H). |
| | N-(4-((S)-4-amino-3,3- | | |
| | dimethylpyrrolidin-1-yl)-1- | | |
| | ((S)-tetrahydrofuran-3-yl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 271 | 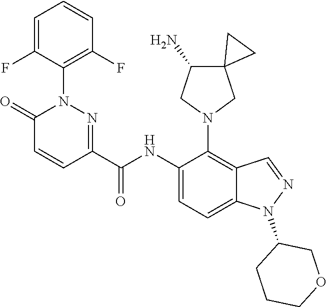 [SMILES: N[C@H]1CN(c2c(NC(=O)c3ccc(=O)n(-c4c(F)cccc4F)n3)ccc3c2cnn3[C@H]2CCCOC2)CC12CC2] | HCOOH salt | LC-MS m/z: 562.6 (M + 1)⁺. ¹H NMR (400 MHz, CD₃OD) δ 8.28 (s, 1H), 8.24 (d, J = 9.9 Hz, 1H), 7.77- 7.61 (m, 2H), 7.32 (dt, J = 9.1, 4.8 Hz, 4H), 4.73-4.60 (m, 2H), 4.23 (dd, J = 10.6, 5.6 Hz, 1H), 4.09-3.94 (m, 3H), 3.85-3.74 (m, 1H), 3.67 (dd, J = 10.6, 2.6 Hz, 1H), 3.53 (td, J = 11.0, 4.2 Hz, 1H), 3.37 (dd, J = 5.5, 2.6 Hz, 1H), 3.13 (d, J = 9.3 Hz, 1H), 2.36- 2.25 (m, 1H), 2.20 (d, J = 12.3 Hz, 1H), 1.92 (tt, J = 8.3, 3.6 Hz, 2H), 1.00 (dt, J = 10.6, 5.5 Hz, 1H), 0.81 (dt, J = 8.9, 5.8 Hz, 1H), 0.77-0.61 (m, 2H). |
| | N-(4-((R)-7-amino-5- | | |
| | azaspiro[2.4]heptan-5-yl)-1- | | |
| | ((S)-tetrahydro-2H-pyran-3- | | |
| | yl)-1H-indazol-5-yl)-1-(2,6- | | |
| | difluorophenyl)-6-oxo-1,6- | | |
| | dihydropyridazine-3- | | |
| | carboxamide | | |
| Example 272 | 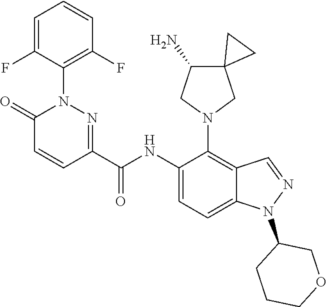 [SMILES: N[C@H]1CN(c2c(NC(=O)c3ccc(=O)n(-c4c(F)cccc4F)n3)ccc3c2cnn3[C@@H]2CCCOC2)CC12CC2] | HCOOH salt | LC-MS m/z: 562.6 M + 1)⁺. ¹H NMR (400 MHz, CD₃OD) δ 8.28 (s, 1H), 8.23 (d, J = 9.9 Hz, 1H), 7.75- 7.63 (m, 2H), 7.36-7.27 (m, 4H), 4.68 (td, J = 10.5, 5.0 Hz, 1H), 4.29- 4.16 (m, 1H), 4.09-3.94 (m, 3H), 3.80 (t, J = 10.6 Hz, 1H), 3.68 (dd, J = 10.6, 2.6 Hz, 1H), 3.53 (td, J = 11.1, 4.1 Hz, 1H), 3.38 (dd, J = 5.5, 2.6 Hz, 1H), 3.13 (d, J = 9.3 Hz, 1H), 2.37- 2.24 (m, 1H), 2.20 (t, J = 7.5 Hz, 1H), 1.92 (tt, J = 9.9, 3.9 Hz, 2H), 1.01 (dt, J = 9.9, 5.6 Hz, 1H), 0.86-0.78 (m, 1H), 0.71 (dddd, J = 17.0, 10.7, 6.1, 4.3 Hz, 2H). |
| | N-(4-((R)-7-amino-5- | | |
| | azaspiro[2.4]heptan-5-yl)-1- | | |
| | ((R)-tetrahydro-2H-pyran-3- | | |
| | yl)-1H-indazol-5-yl)-1-(2,6- | | |
| | difluorophenyl)-6-oxo-1,6- | | |
| | dihydropyridazine-3- | | |
| | carboxamide | | |
| Example 273 | 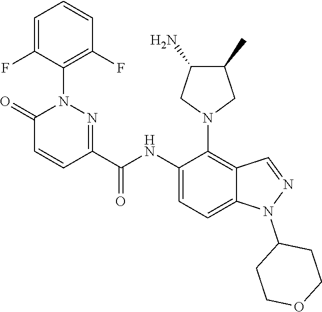 [SMILES: C[C@H]1CN(c2c(NC(=O)c3ccc(=O)n(-c4c(F)cccc4F)n3)ccc3c2cnn3C2CCOCC2)C[C@@H]1N] | HCl salt | LC-MS m/z: 550.2 M + 1)⁺. ¹H NMR (400 MHz, CD₃OD) δ 8.19- 8.10 (m, 2H), 7.78 (d, J = 8.9 Hz, 1H), 7.63-7.51 (m, 1H), 7.30-7.14 (m, 4H), 4.01 (br dd, J = 3.4, 11.4 Hz, 2H), 3.79 (ddd, J = 7.2, 9.6, 14.0 Hz, 3H), 3.63-3.53 (m, 3H), 3.41 (dd, J = 5.9, 10.2 Hz, 1H), 3.02 (dd, J = 5.0, 9.3 Hz, 1H), 2.29-2.10 (m, 3H), 1.83 (br d, J = 10.5 Hz, 1H), 1.88-1.78 (m, 1H), 0.97 (d, J = 6.9 Hz, 1H), 1.03-0.91 (m, 1H), 1.03-0.90 (m, 1H). |
| | N-(4-((3R,4S)-3-amino-4- | | |
| | methylpyrrolidin-1-yl)-1- | | |
| | (tetrahydro-2H-pyran-4-yl)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 274 | 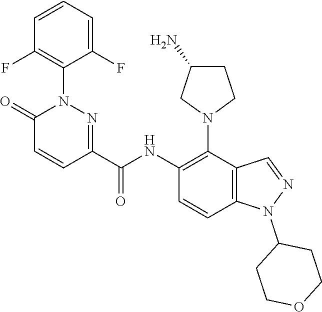 [SMILES: N[C@@H]1CCN(c2c(NC(=O)c3ccc(=O)n(-c4c(F)cccc4F)n3)ccc3c2cnn3C2CCOCC2)C1] | HCl Salt | LC-MS m/z: 536.2 (M + 1)⁺ ¹H NMR (500 MHz, CD₃OD) δ 8.34 (s, 1H), 8.22 (d, J = 9.8 Hz, 1H), 7.77 (br d, J = 8.7 Hz, 1H), 7.74-7.65 (m, 1H), 7.45 (br d, J = 8.7 Hz, 1H), 7.39-7.27 (m, 3H), 4.90-4.82 (m, 1H), 4.12 (br dd, J = 3.5, 11.4 Hz, 2H), 3.97 (br d, J = 9.8 Hz, 2H), 3.85 (br d, J = 7.2 Hz, 1H), 3.77-3.55 (m, 4H), 2.42 (br d, J = 5.2 Hz, 1H), 2.29 (dq, J = 4.3, 12.2 Hz, 2H), 2.12 (br d, J = 4.9 Hz, 1H), 1.96 (br d, J = 12.5 Hz, 2H). |
| | N-[4-[(3R)-3-aminopyrrolidin- | | |
| | 1-yl]-1-tetrahydropyran-4-yl- | | |
| | indazol-5-yl]-1-(2,6- | | |
| | difluorophenyl)-6-oxo- | | |
| | pyridazine-3-carboxamid | | |
| Example 275 | 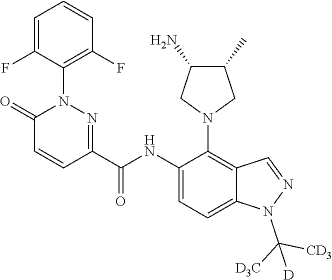 [SMILES: [2H]C([2H])([2H])C([2H])(n1ncc2c(N3C[C@@H](C)[C@@H](N)C3)c(NC(=O)c3ccc(=O)n(-c4c(F)cccc4F)n3)ccc21)C([2H])([2H])[2H]] | HCl salt | LC-MS m/z: 515.2 (M + 1)⁺ ¹H NMR (500 MHz, CD₃OD) δ 8.25- 8.18 (m, 2H), 7.70-7.63 (m, 1H), 7.59 (d, J = 8.9 Hz, 1H), 7.33-7.26 (m, 3H), 7.19 (d, J = 9.0 Hz, 1H), 4.08 (dd, J = 6.0, 10.6 Hz, 1H), 3.88-3.78 (m, 1H), 3.68 (dd, J = 7.6, 9.3 Hz, 1H), 3.58 (dd, J = 3.5, 10.7 Hz, 1H), 3.47 (t, J = 9.0 Hz, 1H), 2.60 (td, J = 7.4, 14.5 Hz, 1H), 1.14 (d, J = 7.2 Hz, 3H). |
| | N-(4-((3R,4R)-3-amino-4- | | |
| | methylpyrrolidin-1-yl)-1- | | |
| | (propan-2-yl-d7)-1H-indazol- | | |
| | 5-yl)-1-(2,6-difluorophenyl)-6- | | |
| | oxo-1,6-dihydropyridazine-3- | | |
| | carboxamide | | |
| Example 276 | 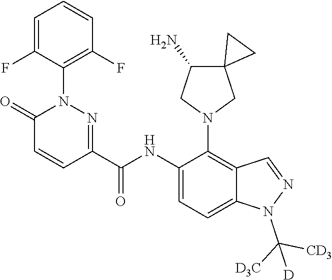 [SMILES: [2H]C([2H])([2H])C([2H])(n1ncc2c(N3C[C@H](N)C4(CC4)C3)c(NC(=O)c3ccc(=O)n(-c4c(F)cccc4F)n3)ccc21)C([2H])([2H])[2H]] | HCl salt | LC-MS m/z: 527.3 (M + 1)⁺ ¹H NMR (500 MHz, CD₃OD) δ 8.29- 8.19 (m, 2H), 7.71-7.63 (m, 2H), 7.35-7.23 (m, 4H), 4.23 (dd, J = 5.6, 10.5 Hz, 1H), 4.03 (d, J = 9.3 Hz, 1H), 3.70-3.65 (m, 1H), 3.38 (br d, J = 2.9 Hz, 1H), 3.12 (d, J = 9.2 Hz, 1H), 1.04- 0.96 (m, 1H), 0.85-0.78 (m, 1H), 0.76-0.66 (m, 2H). |
| | (R)-N-(4-(7-amino-5- | | |
| | azaspiro[2.4]heptan-5-yl)-1- | | |
| | (propan-2-yl-d7)-1H-indazol- | | |
| | 5-yl)-1-(2,6-difluorophenyl)-6- | | |
| | oxo-1,6-dihydropyridazine-3- | | |
| | carboxamide | | |
| Example 277 | 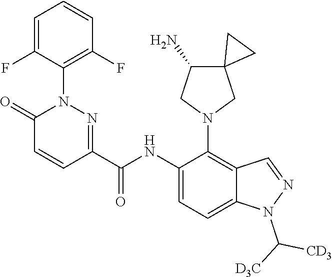 [SMILES: [2H]C([2H])([2H])C(n1ncc2c(N3C[C@H](N)C4(CC4)C3)c(NC(=O)c3ccc(=O)n(-c4c(F)cccc4F)n3)ccc21)C([2H])([2H])[2H]] | HCl salt | LC-MS m/z: 526.3 (M + 1)⁺ ¹H NMR (500 MHz, CD₃OD) δ 8.28- 8.19 (m, 2H), 7.71-7.63 (m, 2H), 7.34-7.23 (m, 4H), 4.91 (br s, 1H), 4.23 (dd, J = 5.6, 10.6 Hz, 1H), 4.03 (d, J = 9.3 Hz, 1H), 3.68 (dd, J = 2.3, 10.7 Hz, 1H), 3.38 (br d, J = 3.2 Hz, 1H), 3.12 (d, J = 9.2 Hz, 1H), 1.03-0.97 (m, 1H), 0.84-0.79 (m, 1H), 0.76-0.67 (m, 2H). |
| | (R)-N-(4-(7-amino-5- | | |
| | azaspiro[2.4]heptan-5-yl)-1- | | |
| | (propan-2-yl-1,1,1,3,3,3-d6)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 278 | 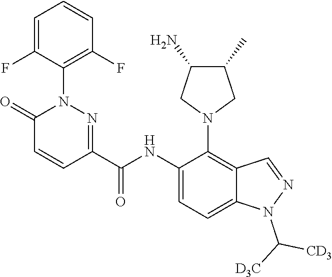 [SMILES: [2H]C([2H])([2H])C(n1ncc2c(N3C[C@@H](C)[C@@H](N)C3)c(NC(=O)c3ccc(=O)n(-c4c(F)cccc4F)n3)ccc21)C([2H])([2H])[2H]] | HCl salt | LC-MS m/z: 514.3 (M + 1)⁺ ¹H NMR (400 MHz, CD₃OD) δ 8.29- 8.13 (m, 2H), 7.71-7.63 (m, 1H), 7.61-7.55 (m, 1H), 7.34-7.25 (m, 3H), 7.19 (d, J = 8.8 Hz, 1H), 4.85-4.84 (m, 1H), 4.08 (dd, J = 5.9, 10.4 Hz, 1H), 3.82 (br d, J = 3.5 Hz, 1H), 3.68 (br t, J = 8.0 Hz, 1H), 3.61-3.54 (m, 1H), 3.51-3.42 (m, 1H), 2.66-2.55 (m, 1H), 1.14 (d, J = 7.2 Hz, 3H). |
| | N-(4-((3R,4R)-3-amino-4- | | |
| | methylpyrrolidin-1-yl)-1- | | |
| | (propan-2-yl-1,1,1,3,3,3-d6)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 279 | 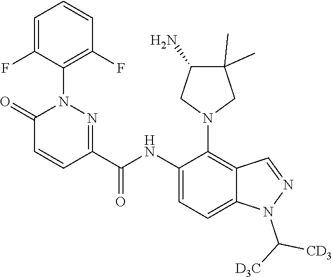 [SMILES: [2H]C([2H])([2H])C(n1ncc2c(N3C[C@H](N)C(C)(C)C3)c(NC(=O)c3ccc(=O)n(-c4c(F)cccc4F)n3)ccc21)C([2H])([2H])[2H]] | HCl salt | LC-MS m/z: 528.3 (M + 1)⁺ ¹H NMR (400 MHz, CD₃OD) δ 8.30- 8.20 (m, 2H), 7.83-7.74 (m, 1H), 7.68 (tt, J = 6.2, 8.6 Hz, 1H), 7.37-7.22 (m, 4H), 5.04-4.98 (m, 1H), 4.09 (dd, J = 6.3, 9.8 Hz, 1H), 3.58 (d, J = 9.2 Hz, 1H), 3.54-3.44 (m, 2H), 3.38 (d, J = 9.2 Hz, 1H), 1.16 (d, J = 11.2 Hz, 6H). |
| | (R)-N-(4-(4-amino-3,3- | | |
| | dimethylpyrrolidin-1-yl)-1- | | |
| | (propan-2-yl-1,1,1,3,3,3-d6)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 280 | 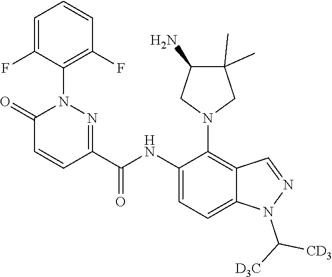 [SMILES: [2H]C([2H])([2H])C(n1ncc2c(N3C[C@@H](N)C(C)(C)C3)c(NC(=O)c3ccc(=O)n(-c4c(F)cccc4F)n3)ccc21)C([2H])([2H])[2H]] | HCl salt | LC-MS m/z: 528.3 (M + 1)⁺ ¹H NMR (400 MHz, CD₃OD) δ 8.29- 8.21 (m, 2H), 7.82-7.74 (m, 1H), 7.68 (tt, J = 6.2, 8.6 Hz, 1H), 7.36-7.22 (m, 4H), 5.02-4.95 (m, 1H), 4.09 (dd, J = 6.3, 9.8 Hz, 1H), 3.58 (d, J = 9.2 Hz, 1H), 3.63-3.55 (m, 1H), 3.53-3.43 (m, 2H), 3.38 (d, J = 9.2 Hz, 1H), 1.16 (d, J = 11.1 Hz, 6H). |
| | (S)-N-(4-(4-amino-3,3- | | |
| | dimethylpyrrolidin-1-yl)-1- | | |
| | (propan-2-yl-1,1,1,3,3,3-d6)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 281 | 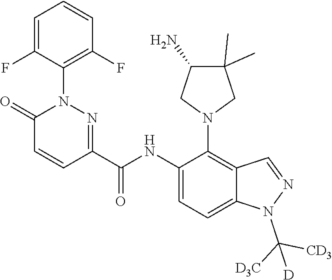 [SMILES: [2H]C([2H])([2H])C([2H])(n1ncc2c(N3C[C@H](N)C(C)(C)C3)c(NC(=O)c3ccc(=O)n(-c4c(F)cccc4F)n3)ccc21)C([2H])([2H])[2H]] | HCl salt | LC-MS m/z: 529.3 (M + 1)⁺ ¹H NMR (400 MHz, CD₃OD) δ 8.29- 8.21 (m, 2H), 7.81-7.74 (m, 1H), 7.68 (tt, J = 6.2, 8.6 Hz, 1H), 7.36-7.24 (m, 4H), 4.14-4.03 (m, 1H), 3.60 (s, 1H), 3.53-3.45 (m, 2H), 3.38 (d, J = 9.3 Hz, 1H), 1.16 (d, J = 11.3 Hz, 6H). |
| | (R)-N-(4-(4-amino-3,3- | | |
| | dimethylpyrrolidin-1-yl)-1- | | |
| | (propan-2-yl-d7)-1H-indazol- | | |
| | 5-yl)-1-(2,6-difluorophenyl)-6- | | |
| | oxo-1,6-dihydropyridazine-3- | | |
| | carboxamide | | |
| Example 282 | 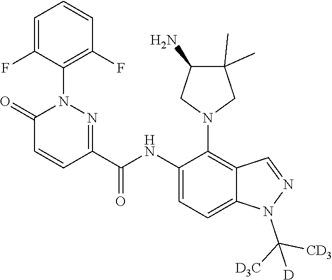 [SMILES: [2H]C([2H])([2H])C([2H])(n1ncc2c(N3C[C@@H](N)C(C)(C)C3)c(NC(=O)c3ccc(=O)n(-c4c(F)cccc4F)n3)ccc21)C([2H])([2H])[2H]] | HCl salt | LC-MS m/z: 529.3 (M + 1)⁺ ¹H NMR (400 MHz, CD₃OD) δ 8.31- 8.21 (m, 2H), 7.83-7.76 (m, 1H), 7.73-7.63 (m, 1H), 7.36-7.22 (m, 4H), 4.14-4.05 (m, 1H), 3.58 (d, J = 9.3 Hz, 1H), 3.54-3.44 (m, 2H), 3.37 (br d, J = 9.3 Hz, 1H), 1.16 (d, J = 12.3 Hz. 6H). |
| | (S)-N-(4-(4-amino-3,3- | | |
| | dimethylpyrrolidin-1-yl)-1- | | |
| | (propan-2-yl-d7)-1H-indaozl- | | |
| | 5-yl)-1-(2,6-difluorophenyl)-6- | | |
| | oxo-1,6-dihydropyridazine-3- | | |
| | carboxamide | | |

### Example 283: N-(4-((3R,4R)-3-amino-4-methylpyrrolidin-1-yl)-1-(2-methoxyethyl)-1H-indazol-5-yl)-1-(2,6-difluorophenyl)-6-oxo-1,6-dihydropyridazine-3-carboxamide

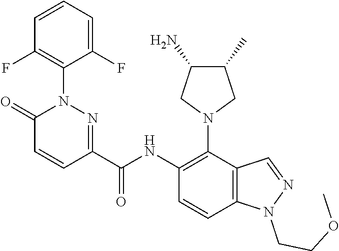 [SMILES: COCCn1ncc2c(N3C[C@@H](C)[C@@H](N)C3)c(NC(=O)c3ccc(=O)n(-c4c(F)cccc4F)n3)ccc21]

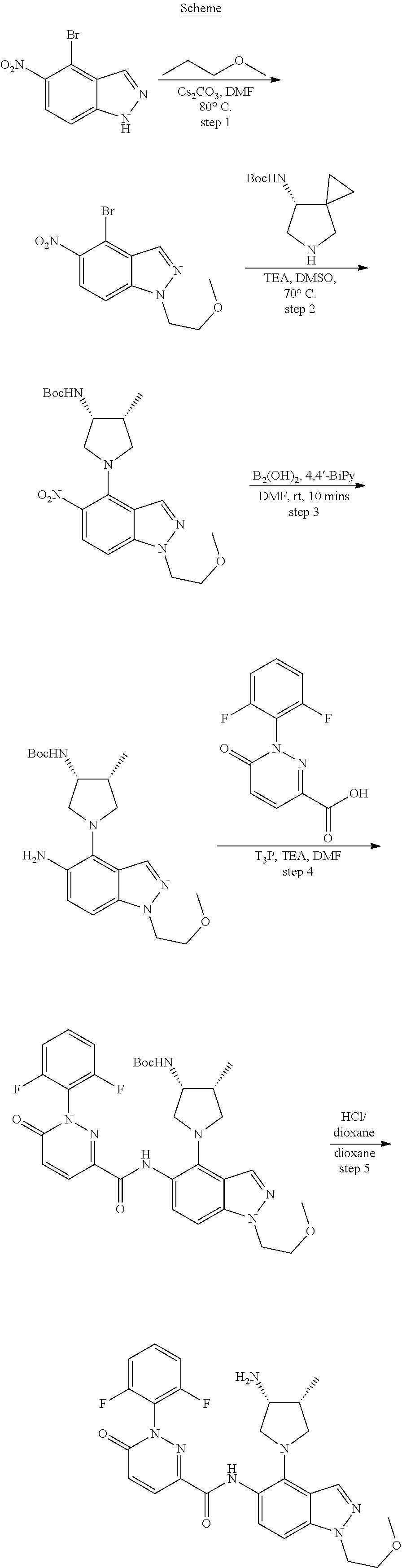

### Step 1: 4-bromo-1-(2-methoxyethyl)-5-nitro-1H-indazole

To a solution of 4-bromo-5-nitro-1H-indazole (200 mg, 826.35 umol, 1 eq) and K₂CO₃ (571.03 mg, 4.13 mmol, 5 eq) in DMF (4 mL) was added 1-bromo-2-methoxy-ethane (229 mg, 1.65 mmol, 154.73 uL, 1.99 eq). The mixture was stirred at 70° C. for 2 hr. LC-MS showed starting material was consumed completely and desired mass was detected. The residue mixture was poured into water (30 ml), extracted with EtOAc (15 ml×3). The combined organic phase was washed with brine over Na₂SO₄, filtered was concentrated into vacuum. The residue was purified by flash silica gel chromatography (ISCO®; 4 g SepaFlash® Silica Flash Column, Eluent of 0^{˜}45% Ethyl acetate/Petroleum ether gradient). 4-bromo-1-(2-methoxyethyl)-5-nitro-indazole (70 mg, Yield: 28.23%) was obtained as a yellow solid.

LC-MS m/z: 300.1 (M+1)⁺

### Step 2: tert-butyl ((3R,4R)-1-(1-(2-methoxyethyl)-5-nitro-1H-indazol-4-yl)-4-methylpyrrolidin-3-yl) carbamate

To a solution of tert-butyl N-[(3R,4R)-4-methylpyrrolidin-3-yl]carbamate (93.99 mg, 469.28 umol, 1.01 eq) in DMSO (1 mL) was added 4-bromo-1-(2-methoxyethyl)-5-nitro-indazole (140 mg, 466.50 umol, 1 eq) and TEA (141.61 mg, 1.40 mmol, 194.79 uL, 3 eq). The mixture was stirred at 100° C. for 2 hr. LC-MS showed starting material was consumed completely and desired mass was detected. The residue mixture was poured into water (30 ml), extracted with EtOAc (15 ml×3). The combined organic phase was washed with brine over Na₂SO₄, filtered was concentrated into vacuo. The residue was purified by flash silica gel chromatography (ISCO®; 4 g SepaFlash® Silica Flash Column, Eluent of 0^{˜}45% Ethyl acetate/Petroleum ether gradient). tert-butyl N-[(3R,4R)-1-[1-(2-methoxyethyl)-5-nitro-indazol-4-yl]-4-methyl-pyrrolidin-3-yl]carbamate (120 mg, Yield: 61.32%) was obtained as a yellow oil.

LC-MS m/z: 420.3 (M+1)⁺

### Step 3: tert-butyl ((3R,4R)-1-(5-amino-1-(2-methoxyethyl)-1H-indazol-4-yl)-4-methylpyrrolidin-3-yl)carbamate

To a solution of tert-butyl N-[(3R,4R)-1-[1-(2-methoxyethyl)-5-nitro-indazol-4-yl]-4-methyl-pyrrolidin-3-yl]carbamate (120 mg, 286.07 umol, 1 eq) in DMF (3 mL) was added 4-(4-pyridyl)pyridine (48 mg, 307.34 umol, 1.07 eq) and hypoboric acid (256.46 mg, 2.86 mmol, 10 eq). The mixture was stirred at 20° C. for 30 min. LC-MS showed starting material was consumed completely and desired mass was detected. The residue mixture was poured into water (20 ml), extracted with EtOAc (15 ml×3). The combined organic phase was washed with brine over Na₂SO₄, filtered was concentrated into vacuum. The residue was used next step directly without further purification. tert-butyl N-[(3R,4R)-1-[5-amino-1-(2-methoxyethyl) indazol-4-yl]-4-methyl-pyrrolidin-3-yl]carbamate (105 mg, Yield: 94.24%) was obtained as a brown oil.

LC-MS m/z: 390.3 (M+1)⁺

### Step 4: tert-butyl ((3R,4R)-1-(5-(1-(2,6-difluorophenyl)-6-oxo-1,6-dihydropyridazine-3-carboxamido)-1-(2-methoxyethyl)-1H-indazol-4-yl)-4-methylpyrrolidin-3-yl)carbamate

To a solution of tert-butyl N-[(3R,4R)-1-[5-amino-1-(2-methoxyethyl)indazol-4-yl]-4-methyl-pyrrolidin-3-yl]carbamate (105 mg, 269.58 umol, 1 eq), 1-(2,6-difluorophenyl)-6-oxo-pyridazine-3-carboxylic acid (68 mg, 269.66 umol, 1 eq) and TEA (137 mg, 1.35 mmol, 188.45 uL, 5.02 eq) in DMF (3 mL) was added T₃P (257 mg, 807.72 umol, 240.19 uL, 3 eq). The mixture was stirred at 20° C. for 15 hr. LC-MS showed starting material was consumed completely and desired mass was detected. The residue mixture was poured into water (20 ml), extracted with EtOAc (15 ml×3). The combined organic phase was washed with brine over Na₂SO₄, filtered was concentrated into vacuum. The residue was used next step directly without further purification. tert-butyl N-[(3R,4R)-1-[5-[[1-(2,6-difluorophenyl)-6-oxo-pyridazine-3-carbonyl]amino]-1-(2-methoxyethyl)indazol-4-yl]-4-methyl-pyrrolidin-3-yl]carbamate (168 mg, Yield: 99.93%) was obtained as a black brown oil.

LC-MS m/z: 624.4 (M+1)⁺

### Step 5: N-(4-((3R,4R)-3-amino-4-methylpyrrolidin-1-yl)-1-(2-methoxyethyl)-1H-indazol-5-yl)-1-(2,6-difluorophenyl)-6-oxo-1,6-dihydropyridazine-3-carboxamide

To a solution of tert-butyl N-[(3R,4R)-1-[5-[[1-(2,6-difluorophenyl)-6-oxo-pyridazine-3-carbonyl]amino]-1-(2-methoxyethyl)indazol-4-yl]-4-methyl-pyrrolidin-3-yl]carbamate (168 mg, 269.38 umol, 1 eq) in dioxane (0.5 mL) was added HCl/dioxane (2 mL). The mixture was stirred at 20° C. for 1 hr. LC-MS showed starting material was consumed completely and desired mass was detected. The reaction mixture was concentrated in vacuum. The residue was purified by prep-HPLC (column: Waters Xbridge BEH C18 100*30 mm*10 um; mobile phase: [water (FA)-ACN]; B %: 14%-44%, 12 min). Example 283 (110 mg, Yield: 78.00%) was obtained as a yellow solid.

LC-MS m/z: 524.3 (M+1)⁺.

¹H NMR (400 MHz, CD₃OD) δ 8.51 (br s, 1H), 8.29-8.13 (m, 2H), 7.71-7.63 (m, 1H), 7.63-7.57 (m, 1H), 7.34-7.23 (m, 3H), 7.19-7.12 (m, 1H), 4.52 (t, J=5.2 Hz, 2H), 4.10-3.92 (m, 1H), 3.81-3.76 (m, 2H), 3.76-3.71 (m, 1H), 3.67-3.59 (m, 1H), 3.56-3.50 (m, 1H), 3.48-3.40 (m, 1H), 3.28-3.19 (m, 3H), 2.60-2.44 (m, 1H), 1.21-1.05 (m, 3H).

| No. | Structure | Salt form | MS and ¹HNMR |
|---|---|---|---|
| Example 284 | 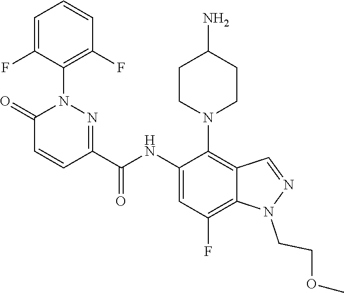 [SMILES: COCCn1ncc2c(N3CCC(N)CC3)c(NC(=O)c3ccc(=O)n(-c4c(F)cccc4F)n3)cc(F)c21] | HCl Salt | LC-MS m/z: 542.3 (M + 1)⁺ 1H NMR (400 MHz, CD₃OD) δ 8.38-8.34 (m, 1H), 8.28-8.23 (m, 1H), 8.28-8.22 (m, 1H), 8.19- 8.12 (m, 1H), 7.77-7.66 (m, 1H), 7.39-7.31 (m, 3H), 4.70 (t, J = 5.2 Hz, 2H), 3.82 (t, J = 5.1 Hz, 2H), 3.30-3.25 (m, 5H), 3.21-3.12 (m, 2H), 1.99-1.89 (m, 2H), 1.74- 1.65 (m, 2H). |
| | N-(4-(4-aminopiperidin-1-yl)-7- | | |
| | fluoro-1-(2-methoxyethyl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 285 | 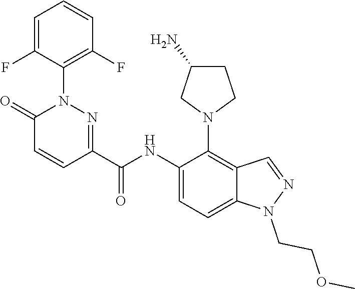 [SMILES: COCCn1ncc2c(N3CC[C@@H](N)C3)c(NC(=O)c3ccc(=O)n(-c4c(F)cccc4F)n3)ccc21] | TFA Salt | LC-MS m/z: 510.2 (M + 1)⁺ ¹H NMR (500 MHz, CD₃OD) δ 8.28- 8.18 (m, 2H), 7.75 (d, J = 8.9 Hz, 1H), 7.70-7.63 (m, 1H), 7.33- 7.24 (m, 4H), 4.54 (t, J = 5.3 Hz, 2H), 3.87 (dd, J = 6.2, 10.0 Hz, 1H), 3.83-3.77 (m, 3H), 3.76-3.69 (m, 1H), 3.53-3.44 (m, 2H), 3.25 (s, 3H), 2.30 (qd, J = 7.3, 14.2 Hz, 1H), 2.01-1.94 (m, 1H). |
| | (R)-N-(4-(3-aminopyrrolidin-1- | | |
| | yl)-1-(2-methoxyethyl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 286 | 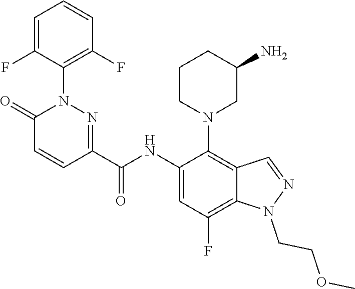 [SMILES: COCCn1ncc2c(N3CCC[C@@H](N)C3)c(NC(=O)c3ccc(=O)n(-c4c(F)cccc4F)n3)cc(F)c21] | HCl Salt | LC-MS m/z: 542.2 (M + 1)⁺ ¹H NMR (400 MHz, CD₃OD) δ 8.45- 8.31 (m, 1H), 8.29-8.23 (m, 2H), 7.82-7.65 (m, 1H), 7.43-7.33 (m, 3H), 4.71 (t, J = 5.2 Hz, 2H), 3.81 (s, 2H), 3.28 (s, 3H), 3.26- 3.22 (m, 1H), 3.23 (br s, 1H), 3.16- 3.09 (m, 1H), 3.08-2.99 (m, 1H), 2.99-2.92 (m, 1H), 1.95-1.83 (m, 1H), 1.83-1.72 (m, 1H), 1.60- 1.44 (m, 1H), 1.40-1.30 (m, 1H). |
| | (R)-N-(4-(3-aminopiperidin-1- | | |
| | yl)-7-fluoro-1-(2-methoxyethyl)- | | |
| | 1H-indazol-5-yl)-1-(2,6- | | |
| | difluorophenyl)-6-oxo-1,6- | | |
| | dihydropyridazine-3- | | |
| | carboxamide | | |
| Example 287 | 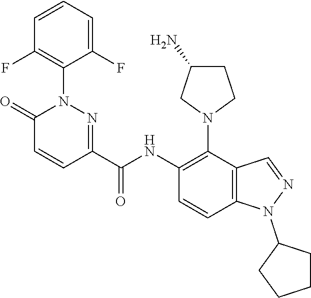 [SMILES: N[C@@H]1CCN(c2c(NC(=O)c3ccc(=O)n(-c4c(F)cccc4F)n3)ccc3c2cnn3C2CCCC2)C1] | HCl Salt | LC-MS m/z: 520.2 (M + 1)⁺ ¹H NMR (400 MHz, CD₃OD) δ 8.26- 8.20 (m, 2H), 7.80-7.76 (m, 1H), 7.72-7.65 (m, 1H), 7.36-7.29 (m, 4H), 5.11 (quin, J = 7.2 Hz, 1H), 3.94-3.82 (m, 2H), 3.82-3.74 (m, 1H), 3.60-3.48 (m, 2H), 2.41- 2.30 (m, 1H), 2.25-1.93 (m, 7H), 1.85-1.73 (m, 2H). |
| | (R)-N-(4-(3-aminopyrrolidin-1- | | |
| | yl)-1-cyclopentyl-1H-indazol-5- | | |
| | yl)-1-(2,6-difluorophenyl)-6- | | |
| | oxo-1,6-dihydropyridazine-3- | | |
| | carboxamide | | |
| Example 288 | 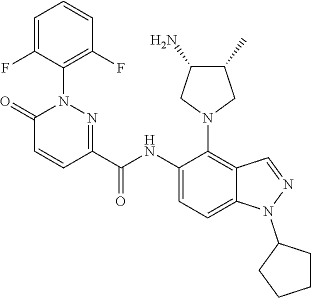 [SMILES: C[C@@H]1CN(c2c(NC(=O)c3ccc(=O)n(-c4c(F)cccc4F)n3)ccc3c2cnn3C2CCCC2)C[C@@H]1N] | HCl Salt | LC-MS m/z: 534.3 (M + 1)⁺ ¹H NMR (400 MHz, CD₃OD) δ 8.46 (d, J = 8.8 Hz, 1H), 8.27 (d, J = 9.8 Hz, 1H), 7.74-7.60 (m, 1H), 7.33 (dd, J = 9.3, 5.5 Hz, 4H), 3.77 (s, 3H), 3.58 (s, 3H), 3.23-3.11 (m, 2H), 2.80 (d, J = 11.6 Hz, 1H), 2.61 (s, 3H), 1.59 (d, J = 19.3 Hz, 2H), 1.43-1.23 (m, 3H). |
| | N-(4-((3R,4R)-3-amino-4- | | |
| | methylpyrrolidin-1-yl)-1- | | |
| | cyclopentyl-1H-indazol-5-yl)-1- | | |
| | (2,6-difluorophenyl)-6-oxo-1,6- | | |
| | dihydropyridazine-3- | | |
| | carboxamide34 | | |
| Example 289 | 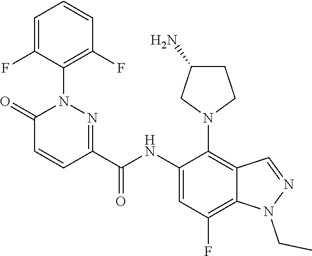 [SMILES: CCn1ncc2c(N3CC[C@@H](N)C3)c(NC(=O)c3ccc(=O)n(-c4c(F)cccc4F)n3)cc(F)c21] | HCl Salt | LC-MS m/z: 498.3 (M + 1)⁺ ¹H NMR (500 MHz, CD₃OD) δ 8.31- 8.19 (m, 2H), 8.03 (d, J = 13.1 Hz, 1H), 7.78-7.63 (m, 1H), 7.42- 7.28 (m, 3H), 4.60 (q, J = 7.3 Hz, 2H), 3.83-3.69 (m, 2H), 3.62- 3.56 (m, 1H), 3.47-3.34 (m, 2H), 2.34-2.20 (m, 1H), 2.02-1.90 (m, 1H), 1.48 (t, J = 7.2 Hz, 3H). |
| | (R)-N-(4-(3-aminopyrrolidin-1- | | |
| | yl)-1-ethyl-7-fluoro-1H-indazol- | | |
| | 5-yl)-1-(2,6-difluorophenyl)-6- | | |
| | oxo-1,6-dihydropyridazine-3- | | |
| | carboxamide | | |
| Example 290 | 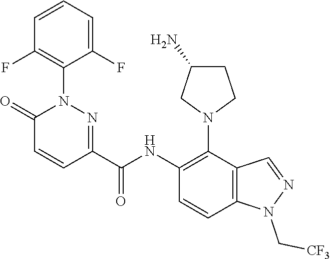 [SMILES: N[C@@H]1CCN(c2c(NC(=O)c3ccc(=O)n(-c4c(F)cccc4F)n3)ccc3c2cnn3CC(F)(F)F)C1] | HCl Salt | LC-MS m/z: 534.1 (M + 1)⁺ ¹H NMR (500 MHz, CD₃OD) δ 8.37 (s, 1H), 8.24-8.22 (m, 1H), 7.78- 7.76 (m, 1H), 7.71-7.58 (m, 1H), 7.35-7.30 (m, 4H), 5.22-5.17 (m, 2H), 3.95-3.92 (m, 1H), 3.90- 3.84 (m, 2H), 3.58-3.50 (m, 2H), 2.38-2.32 (m, 1H), 2.10-2.02 (m, 1H). |
| | (R)-N-(4-(3-aminopyrrolidin-1- | | |
| | yl)-1-(2,2,2-trifluoroethyl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 291 | 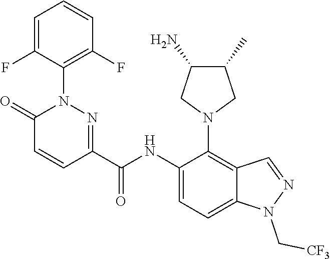 [SMILES: C[C@@H]1CN(c2c(NC(=O)c3ccc(=O)n(-c4c(F)cccc4F)n3)ccc3c2cnn3CC(F)(F)F)C[C@@H]1N] | HCOOH Salt | LC-MS m/z: 548.3 (M + 1)⁺ ¹H NMR (400 MHz, CD₃OD) δ 8.34 (d, J = 1.0 Hz, 1H), 8.20 (d, J = 9.8 Hz, 1H), 7.70-7.62 (m, 1H), 7.60 (d, J = 8.9 Hz, 1H), 7.33-7.23 (m, 3H), 7.16 (d, J = 8.8 Hz, 1H), 5.14 (q, J = 8.7 Hz, 2H), 4.05 (dd, J = 10.4, 5.8 Hz, 1H), 3.73 (td, J = 6.0, 3.4 Hz, 1H), 3.70-3.63 (m, 1H), 3.60-3.45 (m, 2H), 2.54 (hept, J = 7.2 Hz, 1H), 1.11 (d, J = 7.0 Hz, 3H). |
| | N-(4-((3R,4R)-3-amino-4- | | |
| | methylpyrrolidin-1-yl)-1-(2,2,2- | | |
| | trifluoroethyl)-1H-indazol-5-yl)- | | |
| | 1-(2,6-difluorophenyl)-6-oxo- | | |
| | 1,6-dihydropyridazine-3- | | |
| | carboxamide | | |
| Example 292 | 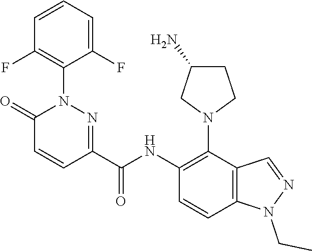 [SMILES: CCn1ncc2c(N3CC[C@@H](N)C3)c(NC(=O)c3ccc(=O)n(-c4c(F)cccc4F)n3)ccc21] | HCl Salt | LC-MS m/z: 480.3 (M + 1)⁺ ¹H NMR (400 MHz, CD₃OD) δ 8.26 (s, 1H), 8.23 (d, J = 9.9 Hz, 1H), 7.76 (d, J = 8.9 Hz, 1H), 7.69 (tt, J = 6.2, 8.6 Hz, 1H), 7.39-7.24 (m, 4H), 4.45 (q, J = 7.2 Hz, 2H), 3.96- 3.89 (m, 1H), 3.88-3.83 (m, 1H), 3.82-3.73 (m, 1H), 3.60-3.48 (m, 2H), 2.41-2.27 (m, 1H), 2.09- 1.97 (m, 1H), 1.46 (t, J = 7.2 Hz, 3H). |
| | (R)-N-(4-(3-aminopyrrolidin-1- | | |
| | yl)-1-ethyl-1H-indazol-5-yl)-1- | | |
| | (2,6-difluorophenyl)-6-oxo-1,6- | | |
| | dihydropyridazine-3- | | |
| | carboxamide | | |
| Example 293 | 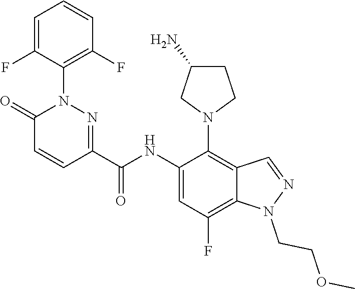 [SMILES: COCCn1ncc2c(N3CC[C@@H](N)C3)c(NC(=O)c3ccc(=O)n(-c4c(F)cccc4F)n3)cc(F)c21] | HCl Salt | LC-MS m/z: 528.2 (M + 1)⁺ ¹H NMR (500 MHz, CD₃OD) δ 8.31 (d, J = 2.0 Hz, 1H), 8.23 (d, J = 9.8 Hz, 1H), 8.02 (d, J = 13.1 Hz, 1H), 7.77-7.66 (m, 1H), 7.41-7.28 (m, 3H), 4.71 (t, J = 5.3 Hz, 2H), 3.82 (t, J = 5.1 Hz, 2H), 3.78-3.70 (m, 2H), 3.63-3.57 (m, 1H), 3.45- 3.35 (m, 2H), 2.35-2.21 (m, 1H), 2.05-1.90 (m, 1H). |
| | (R)-N-(4-(3-aminopyrrolidin-1- | | |
| | yl)-7-fluoro-1-(2- | | |
| | methoxyethyl)-1H-indazol-5- | | |
| | yl)-1-(2,6-difluorophenyl)-6- | | |
| | oxo-1,6-dihydropyridazine-3- | | |
| | carboxamide | | |
| Example 294 | 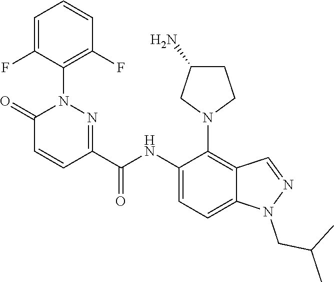 [SMILES: CC(C)Cn1ncc2c(N3CC[C@@H](N)C3)c(NC(=O)c3ccc(=O)n(-c4c(F)cccc4F)n3)ccc21] | HCl Salt | LC-MS m/z: 508.3 (M + 1)⁺ ¹H NMR (400 MHz, CD₃OD) δ 8.31 (s, 1H), 8.22 (d, J = 9.8 Hz, 1H), 7.74 (d, J = 8.9 Hz, 1H), 7.71-7.64 (m, 1H), 7.36-7.27 (m, 4H), 4.22 (d, J = 7.3 Hz, 2H), 4.00-3.86 (m, 2H), 3.85-3.76 (m, 1H), 3.59 (ddd, J = 4.2, 9.4, 18.7 Hz, 2H), 2.42-2.26 (m, 2H), 2.12-2.01 (m, 1H), 0.93 (d, J = 6.7 Hz, 6H). |
| | (R)-N-(4-(3-aminopyrrolidin-1- | | |
| | yl)-1-isobutyl-1H-indazol-5-yl)- | | |
| | 1-(2,6-difluorophenyl)-6-oxo- | | |
| | 1,6-dihydropyridazine-3- | | |
| | carboxamide | | |
| Example 295 | 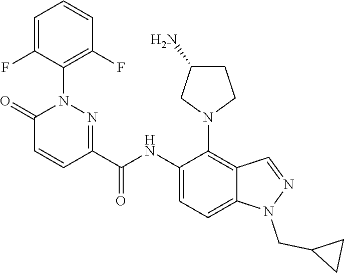 [SMILES: N[C@@H]1CCN(c2c(NC(=O)c3ccc(=O)n(-c4c(F)cccc4F)n3)ccc3c2cnn3CC2CC2)C1] | HCl Salt | LC-MS m/z: 506.3 (M + 1)⁺ ¹H NMR (400 MHz, CD₃OD) δ 8.29 (s, 1H), 8.22 (d, 1H), 7.76 (m, 1H), 7.68 (m, 1H), 7.35-7.30 (m, 4H), 4.30 (d, 2H), 3.93 (m, 2H), 3.61 (m, 1H), 3.55 (m, 2H), 2.36 (m, 1H), 2.05 (m, 1H), 1.34 (m, 1H), 0.58-0.54 (m, 2H), 0.45-0.42 (m, 2H). |
| | (R)-N-(4-(3-aminopyrrolidin-1- | | |
| | yl)-1-(cyclopropylmethyl)-1H- | | |
| | indazol-5-yl)-1-(2,6- | | |
| | difluorophenyl)-6-oxo-1,6- | | |
| | dihydropyridazine-3- | | |
| | carboxamide | | |
| Example 296 | 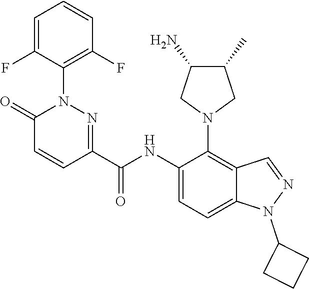 [SMILES: C[C@@H]1CN(c2c(NC(=O)c3ccc(=O)n(-c4c(F)cccc4F)n3)ccc3c2cnn3C2CCC2)C[C@@H]1N] | HCl Salt | LC-MS m/z: 520.3 (M + 1)⁺ ¹H NMR (400 MHz, CD₃OD) δ 8.28- 8.18 (m, 2H), 7.66 (tt, J = 6.2, 8.6 Hz, 1H), 7.58 (d, J = 8.8 Hz, 1H), 7.34-7.24 (m, 3H), 7.18 (d, J = 8.9 Hz, 1H), 5.17 (quin, J = 8.3 Hz, 1H), 4.08 (dd, J = 6.0, 10.7 Hz, 1H), 3.86- 3.79 (m, 1H), 3.68 (dd, J = 7.6, 9.3 Hz, 1H), 3.60-3.55 (m, 1H), 3.49- 3.45 (m, 1H), 2.71 (ddd, J = 4.9, 9.1, 12.0 Hz, 2H), 2.65 (br s, 1H), 2.57-2.46 (m, 2H), 1.99-1.95 (m, 1H), 2.03-1.89 (m, 2H), 1.14 (d, J = 7.2 Hz, 3H). |
| | N-(4-((3R,4R)-3-amino-4- | | |
| | methylpyrrolidin-1-yl)-1- | | |
| | cyclobutyl-1H-indazol-5-yl)-1- | | |
| | (2,6-difluorophenyl)-6-oxo-1,6- | | |
| | dihydropyridazine-3- | | |
| | carboxamide | | |
| Example 297 | 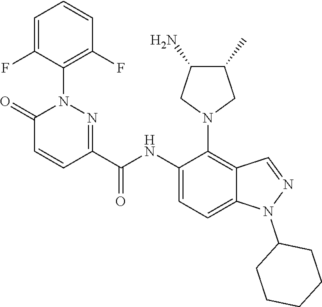 [SMILES: C[C@@H]1CN(c2c(NC(=O)c3ccc(=O)n(-c4c(F)cccc4F)n3)ccc3c2cnn3C2CCCCC2)C[C@@H]1N] | HCl Salt | LC-MS m/z: 548.3 (M + 1)⁺ ¹H NMR (400 MHz, CD₃OD) δ 8.24- 8.18 (m, 2H), 7.71-7.57 (m, 2H), 7.34-7.18 (m, 4H), 4.55-4.43 (m, 1H), 4.07 (dd, J = 6.0, 10.7 Hz, 1H), 3.87-3.79 (m, 1H), 3.67 (dd, J = 7.6, 9.3 Hz, 1H), 3.57 (dd, J = 3.6, 10.6 Hz, 1H), 3.46 (t, J = 8.9 Hz, 1H), 2.64-2.53 (m, 1H), 2.04- 1.89 (m, 6H), 1.80 (br d, J = 13.1 Hz, 1H), 1.66-1.50 (m, 2H), 1.42- 1.26 (m, 1H), 1.14 (d, J = 7.0 Hz, 3H). |
| | N-(4-((3R,4R)-3-amino-4- | | |
| | methylpyrrolidin-1-yl)-1- | | |
| | cyclohexyl-1H-indazol-5-yl)-1- | | |
| | (2,6-difluorophenyl)-6-oxo-1,6- | | |
| | dihydropyridazine-3- | | |
| | carboxamide | | |

### Example 298: N-[4-[(3R,4R)-3-amino-4-methyl-pyrrolidin-1-yl]-1-cyclopropyl-indazol-5-yl]-1-(2,6-difluorophenyl)-6-oxo-pyridazine-3-carboxamide

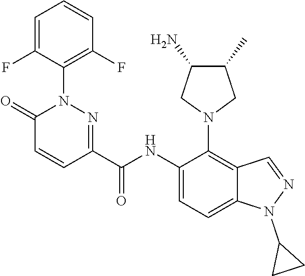 [SMILES: C[C@@H]1CN(c2c(NC(=O)c3ccc(=O)n(-c4c(F)cccc4F)n3)ccc3c2cnn3C2CC2)C[C@@H]1N]

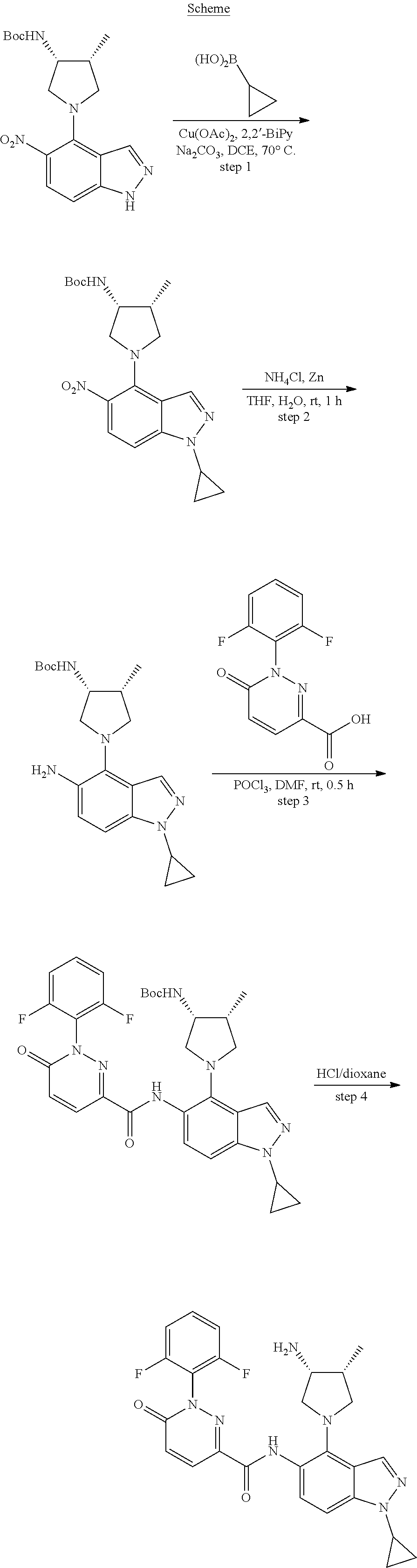

### Step 1: tert-butyl N-[(3R,4R)-1-(1-cyclopropyl-5-nitro-indazol-4-yl)-4-methyl-pyrrolidin-3-yl]carbamate

To a solution of tert-butyl N-[(3R,4R)-4-methyl-1-(5-nitro-1H-indazol-4-yl)pyrolidin-3-yl]carbamate (200 mg, 553.41 umol, 1 eq), cyclopropylboronic acid (142.61 mg, 1.66 mmol, 3 eq), 2-(2-pyridyl)pyridine (86.43 mg, 553.41 umol, 1 eq) and disodium carbonate (175.97 mg, 1.66 mmol, 3 eq) in DCE (6 mL) was added Cu(OAc)₂ (110.57 mg, 608.75 μmol, 1.1 eq). The mixture was stirred at 70° C. for 2 hr. LC-MS showed one main peak with desired mass was detected. The reaction mixture was poured into water (50 mL), extracted with EtOAc (30 mL×3). The organic layer was washed with brine (30 mL), dried over Na₂SO₄ and filtered. The filtrate was concentrated and purified by flash silica gel chromatography (ISCO®; 4 g SepaFlash® Silica Flash Column, eluent of 30% ethyl acetate/pet. ether gradient). Compound tert-butyl N-[(3R,4R)-1-(1-cyclopropyl-5-nitro-indazol-4-yl)-4-methyl-pyrrolidin-3-yl]carbamate(160 mg, Yield: 72.02%) was obtained as a yellow solid.

LC-MS m/z: 402.2 (M+1)⁺.

### Step 2: tert-butyl N-[(3R,4R)-1-(5-amino-1-cyclopropyl-indazol-4-yl)-4-methyl-pyrrolidin-3-yl]carbamate

To a solution of tert-butyl N-[(3R,4R)-1-(1-cyclopropyl-5-nitro-indazol-4-yl)-4-methyl-pyrrolidin-3-yl]carbamate (160 mg, 398.55 umol, 1 eq) in THF (10 mL) and H₂O (5 mL) was added NH₄Cl (170.55 mg, 3.19 mmol, 8 eq) and Zn (208.49 mg, 3.19 mmol, 8 eq). The mixture was stirred at 25° C. for 1 hr. LC-MS showed one main peak with desired mass was detected. The reaction mixture was poured into water (30 mL), extracted with EtOAc (20 mL×3). The organic layer was washed with brine (30 mL), dried over Na₂SO₄ and filtered to give a product. Without further purification. tert-butyl N-[(3R,4R)-1-(5-amino-1-cyclopropyl-indazol-4-yl)-4-methyl-pyrrolidin-3-yl]carbamate (105 mg, Yield: 70.92%) was obtained as an invisible green solid.

LC-MS m/z: 372.2 (M+1)⁺.

### Step 3: tert-butyl N-[(3R,4R)-1-[1-cyclopropyl-5-[[1-(2,6-difluorophenyl)-6-oxo-pyridazine-3-carbonyl]amino]indazol-4-yl]-4-methyl-pyrrolidin-3-yl]carbamate

To a solution of 1-(2,6-difluorophenyl)-6-oxo-pyridazine-3-carboxylic acid (71.28 mg, 282.66 umol, 1 eq) and tert-butyl N-[(3R,4R)-1-(5-amino-1-cyclopropyl-indazol-4-yl)-4-methyl-pyrrolidin-3-yl]carbamate (105 mg, 282.66 umol, 1 eq) in Py (2 mL) was added POCl₃ (43.34 mg, 282.66 umol, 26.27 uL, 1 eq). The mixture was stirred at 25° C. for 0.5 hr. LC-MS showed one main peak with desired mass was detected. The reaction mixture was poured into water (50 mL), extracted with EtOAc (30 mL×3). The organic layer was washed with brine (30 mL), dried over Na₂SO₄ and filtered. The filtrate was concentrated and purified by flash silica gel chromatography (ISCO®; 4 g SepaFlash® Silica Flash Column, eluent of 35% ethyl acetate/pet. ether gradient). Compound tert-butyl N-[(3R,4R)-1-[1-cyclopropyl-5-[[1-(2,6-difluorophenyl)-6-oxo-pyridazine-3-carbonyl]amino]indazol-4-yl]-4-methyl-pyrrolidin-3-yl]carbamate (140 mg, Yield: 81.78%) was obtained as a yellow solid.

LC-MS m/z: 606.3 (M+1)⁺.

### Step 4: N-[4-[(3R,4R)-3-amino-4-methyl-pyrrolidin-1-yl]-1-cyclopropyl-indazol-5-yl]-1-(2,6-difluorophenyl)-6-oxo-pyridazine-3-carboxamide

To a solution of tert-butyl N-[(3R,4R)-1-[1-cyclopropyl-5-[[1-(2,6-difluorophenyl)-6-oxo-pyridazine-3-carbonyl]amino]indazol-4-yl]-4-methyl-pyrrolidin-3-yl]carbamate (140 mg, 231.16 umol, 1 eq) in dioxane (5 mL) and HCl/dioxane (10 mL). The mixture was stirred at 25° C. for 1.5 hr. LC-MS showed one main peak with desired mass was detected. The reaction mixture was concentrated under reduced pressure to remove solvent. The crude product was purified by pre-HPLC (Boston Prime C18 150×30 mm×5 μm, water (HCl)-ACN, Begin B 19, End B 39, Gradient Time (min) 2, 100% B Hold Time (min) 10, FlowRate (ml/min) 25). Example 298 (28.39 mg, Yield: 24.29%) was obtained as a yellow solid.

LC-MS m/z: 506.3 (M+1).

¹H NMR (400 MHz, CD₃OD) δ 8.25-8.18 (i, 2H), 7.73-7.63 (m, 1H), 7.63-7.59 (m, 1H), 7.36-7.26 (m, 4H), 4.10 (dd, =5.9, 10.7 Hz, 1H), 3.88-3.81 (9, 1H), 3.74-3.67 (m, 1H), 3.65-3.56 (m, 2H), 3.53-3.45 (m, 1H), 2.67-2.56 (m, 1H), 1.24-1.16 (d, 4H), 1.14 (br s, 3H).

| No. | Structure | Salt form | MS and ¹HNMR |
|---|---|---|---|
| Example 299 | 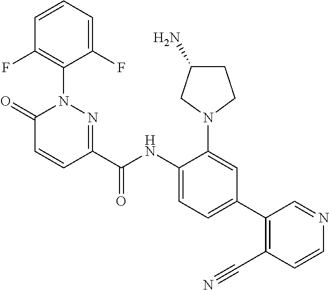 [SMILES: N#Cc1ccncc1-c1ccc(NC(=O)c2ccc(=O)n(-c3c(F)cccc3F)n2)c(N2CC[C@@H](N)C2)c1] N- (4-((3R,4S)-3-amino-4- methylpyrrolidin-1-yl)-1- cyclopropyl-1H-indazol-5-yl)- 1-(2,6-difluorophenyl)-6-oxo- 1,6-dihydropyridazine-3- carboxamide | HCl Salt | LC-MS m/z: 506.3 (M + 1)⁺ ¹H NMR (500 MHz, CD₃OD) δ 8.24 (d, J = 9.8 Hz, 1H), 8.19 (s, 1H), 7.86 (d, J = 9.0 Hz, 1H), 7.72-7.63 (m, 1H), 7.40 (d, J = 9.0 Hz, 1H), 7.36- 7.28 (m, 3H), 3.99-3.83 (m, 2H), 3.63 (td, J = 3.4, 7.0 Hz, 1H), 3.51 (dd, J = 5.6, 9.9 Hz, 1H), 3.40-3.37 (m, 1H), 3.16 (dd, J = 5.2, 9.5 Hz, 1H), 2.39-2.29 (m, 1H), 1.24- 1.19 (m, 2H), 1.17-1.13 (m, 2H), 1.10 (d, J = 6.9 Hz, 3H). |
| Example 300 | 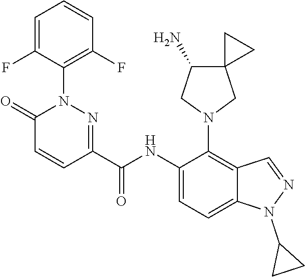 [SMILES: N[C@H]1CN(c2c(NC(=O)c3ccc(=O)n(-c4c(F)cccc4F)n3)ccc3c2cnn3C2CC2)CC12CC2] (R)-N-(4-(7-amino-5- azaspiro[2.4]heptan-5-yl)-1- cyclopropyl-1H-indazol-5-yl)- 1-(2,6-difluorophenyl)-6-oxo- 1,6-dihydropyridazine-3- carboxamide | HCl Salt | LC-MS m/z: 518.3 (M + 1)⁺ ¹H NMR (500 MHz, CD₃OD) δ 8.24- 8.17 (m, 2H), 7.71-7.61 (m, 2H), 7.36-7.24 (m, 4H), 4.27-4.20 (m, 1H), 4.05-4.01 (m, 1H), 3.69-3.57 (m, 2H), 3.39-3.37 (m, 1H), 3.17- 3.08 (m, 1H), 1.21-1.17 (m, 2H), 1.16-1.10 (m, 2H), 1.04-0.96 (m, 1H), 0.86-0.78 (m, 1H), 0.76-0.66 (m, 2H). |
| Example 301 | 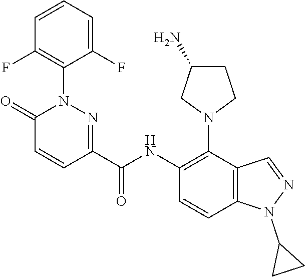 [SMILES: N[C@@H]1CCN(c2c(NC(=O)c3ccc(=O)n(-c4c(F)cccc4F)n3)ccc3c2cnn3C2CC2)C1] (R)-N-(4-(3-aminopyrrolidin- 1-yl)-1-cyclopropyl-1H- indazol-5-yl)-1-(2,6- difluorophenyl)-6-oxo-1,6- dihydropyridazine-3- carboxamide | HCl Salt | LC-MS m/z: 492.2 (M + 1)⁺ ¹H NMR (500 MHz, CD₃OD) δ 8.23 (d, J = 10.5 Hz, 1H), 8.19 (s, 1H), 7.78 (d, J = 8.9 Hz, 1H), 7.74-7.65 (m, 1H), 7.38 (d, J = 8.9 Hz, 1H), 7.36- 7.28 (m, 3H), 3.94-3.88 (m, 1H), 3.87-3.82 (m, 1H), 3.80-3.70 (m, 1H), 3.63 (tt, J = 3.6, 7.0 Hz, 1H), 3.56- 3.45 (m, 2H), 2.39-2.29 (m, 1H), 2.06-1.96 (m, 1H), 1.22-1.13 (m, 4H). |

### Example 302: N-[4-[(7R)-7-amino-5-azaspiro[2.4]heptan-5-yl]-1-tert-butyl-indazol-5-yl]-1-(2,6-difluorophenyl)-6-oxo-pyridazine-3-carboxamide

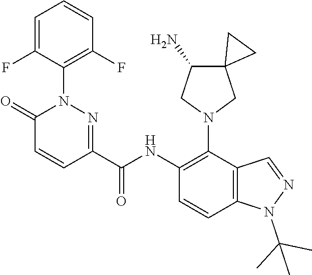 [SMILES: CC(C)(C)n1ncc2c(N3C[C@H](N)C4(CC4)C3)c(NC(=O)c3ccc(=O)n(-c4c(F)cccc4F)n3)ccc21]

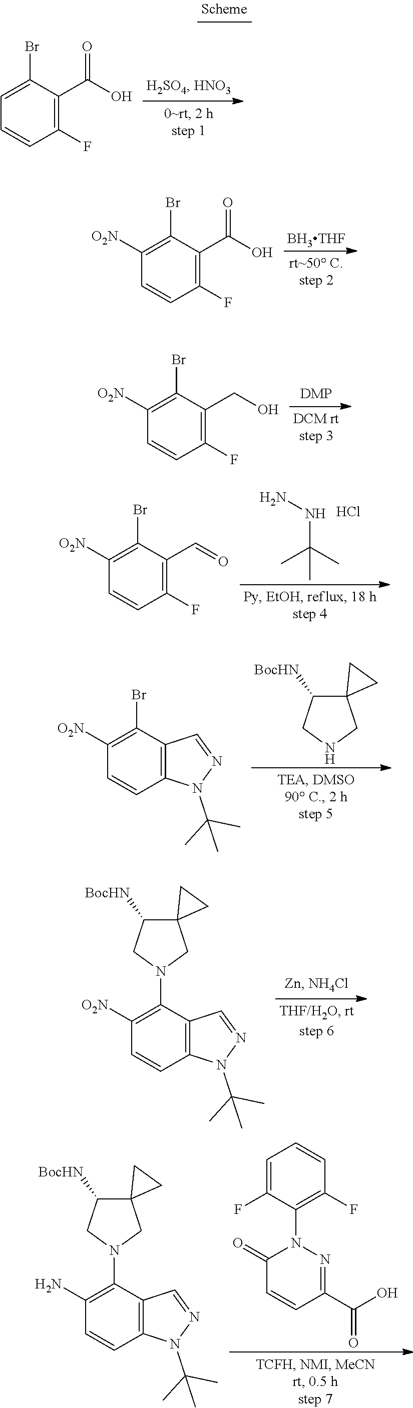

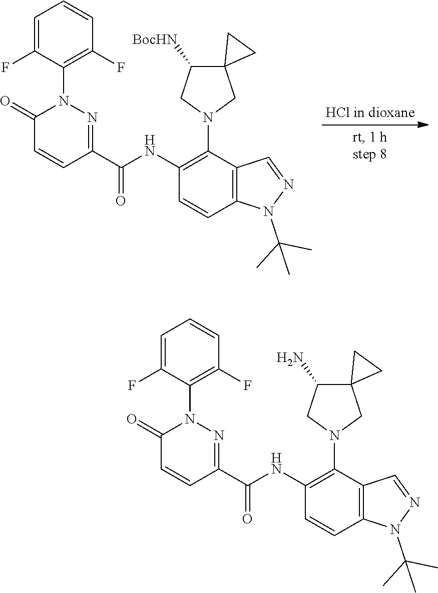 [SMILES: CC(C)(C)OC(=O)N[C@H]1CN(c2c(NC(=O)c3ccc(=O)n(-c4c(F)cccc4F)n3)ccc3c2cnn3C(C)(C)C)CC12CC2 ; CC(C)(C)n1ncc2c(N3C[C@H](N)C4(CC4)C3)c(NC(=O)c3ccc(=O)n(-c4c(F)cccc4F)n3)ccc21]

### Step 1: 2-bromo-6-fluoro-3-nitro-benzoic acid

To a solution of 2-bromo-6-fluoro-benzoic acid (5 g, 22.83 mmol, 1 eq) in sulfuric acid (15 mL) was added HNO₃ (3.76 g, 59.67 mmol, 2.69 mL, 2.61 eq) at 0° C. The mixture was stirred at 20° C. for 2 hr. TLC showed starting material was consumed and new spot formed. The reaction mixture was poured into water 80 ml, filtered and the solid washed with water and dried under vacuum. The residue was used next step without further purification. 2-bromo-6-fluoro-3-nitro-benzoic acid (4.2 g, Yield: 69.68%) was obtained as white solid. 1H NMR (500 MHz, CD₃OD) δ=8.03 (dd, J=5.1, 9.1 Hz, 1H), 7.47 (dd, J=8.2, 8.9 Hz, 1H).

### Step 2: (2-bromo-6-fluoro-3-nitro-phenyl)methanol)

To a solution of 2-bromo-6-fluoro-3-nitro-benzoic acid (3.8 g, 14.39 mmol, 1 eq) in THF (40 mL) was added BH₃. THF (1 M, 28.79 mL, 2 eq). The mixture was stirred at 60° C. for 2 h. TLC showed starting material was consumed and new spot formed. The reaction mixture was added to H₂O 30 mL and extracted with EtOAc (30 mL×3). The combined organic layers were washed with brine, dried over Na₂SO₄, filtered and concentrated under reduced pressure to give a residue. The residue was used next step without further purification. (2-bromo-6-fluoro-3-nitro-phenyl)methanol (3.59 g, Yield: 99.76%) was obtained as a white solid. 1H NMR (400 MHz, CDCl₃) δ 7.73 (dd, J=5.1, 8.9 Hz, 1H), 7.15 (t, J=8.5 Hz, 1H), 4.88 (d, J=2.5 Hz, 1H), 4.86 (br s, 1H), 4.90-4.85 (m, 1H).

### Step 3: 2-bromo-6-fluoro-3-nitro-benzaldehyde

To a solution of (2-bromo-6-fluoro-3-nitro-phenyl)methanol (1.0 g, 4.00 mmol, 1 eq) in DCM (10 mL) was added DMP (1.87 g, 4.40 mmol, 1.36 mL, 1.1 eq). The mixture was stirred at 20° C. for 2 hr. TLC showed most of starting material was consumed and desired mass was detected. The mixture was added to saturated 10 mL aqueous NaHCO₃. The mixture was filtered and filtrate was extracted with DCM (15 mL×3). The combined organic layers were washed with brine, dried over Na₂SO₄, filtered and concentrated under reduced pressure to give a residue. The residue was purified by flash silica gel chromatography (ISCO®; 20 g SepaFlash® Silica Flash Column, Eluent of 0^{˜}10% Ethyl acetate/Petroleum ethergradient). Compound 2-bromo-6-fluoro-3-nitro-benzaldehyde (764 mg, Yield: 77.02%) was obtained as a white solid. 1H NMR (500 MHz, CDCl₃) 5=10.36-10.29 (m, 1H), 10.36-10.29 (m, 1H), 7.97 (br dd, J=4.7, 9.0 Hz, 1H), 7.38-7.28 (m, 1H).

### Step 4: 4-bromo-1-tert-butyl-5-nitro-indazole

To a solution of 2-bromo-6-fluoro-3-nitro-benzaldehyde (700 mg, 2.82 mmol, 1 eq) and Py (1.12 g, 14.11 mmol, 1.14 mL, 5 eq) in Ethanol (7 mL) was added tert-butylhydrazine (422.06 mg, 3.39 mmol, 1.2 eq, HCl). The mixture was stirred at 140° C. by microwave for 1 hr. LCMS showed starting material was consumed and desired mass was detected. The reaction mixture was added to H₂O 15 mL and extracted with EtOAc (15 mL×3). The combined organic layers were washed with brine, dried over Na₂SO₄, filtered and concentrated under reduced pressure to give a residue. The residue was purified by flash silica gel chromatography (ISCO®; 4 g SepaFlash® Silica Flash Column, Eluent of 0^{˜}10% Ethyl acetate/Petroleum ether gradient). Compound 4-bromo-1-tert-butyl-5-nitro-indazole (350 mg, Yield: 41.59%) was obtained as yellow solid.

¹H NMR (500 MHz, CDCl₃) 5=8.19 (s, 1H), 7.96 (d, J=9.2 Hz, 1H), 7.69 (d, J=9.2 Hz, 1H), 1.79 (s, 9H).

### Step 5: tert-butyl N-[(7R)-5-(1-tert-butyl-5-nitro-indazol-4-yl)-5-azaspiro[2.4]heptan-7-yl]carbamate

To a solution of 4-bromo-1-tert-butyl-5-nitro-indazole (300 mg, 1.01 mmol, 1 eq) and tert-butyl N-[(7R)-5-azaspiro[2.4]heptan-7-yl]carbamate (214.29 mg, 1.01 mmol, 1 eq) in DMSO (5 mL) was added TEA (305.47 mg, 3.02 mmol, 420.17 uL, 3 eq). The mixture was stirred at 90° C. for 1 hr. LCMS showed starting material was consumed and desired mass was detected. The reaction mixture was added to H₂O 20 mL and extracted with EtOAc (20 mL×3). The combined organic layers were washed with brine, dried over Na₂SO₄, filtered and concentrated under reduced pressure to give a residue. The residue was used next step without further purification. Compound tert-butyl N-[(7R)-5-(1-tert-butyl-5-nitro-indazol-4-yl)-5-azaspiro [2.4]heptan-7-yl]carbamate (432 mg, Yield: 99.95%) was obtained as yellow solid.

LC-MS m/z: 430.3 (M+1)⁺.

¹H NMR (400 MHz, CDCl₃) 5=8.17 (s, 1H), 7.87 (d, J=9.4 Hz, 1H), 7.00 (d, J=9.4 Hz, 1H), 4.80 (br s, 1H), 4.14 (s, 1H), 4.12 (br d, J=3.1 Hz, 1H), 4.02 (d, J=10.6 Hz, 1H), 3.93 (br s, 1H), 3.63 (br d, J=8.9 Hz, 1H), 3.05 (br d, J=10.8 Hz, 1H), 1.78 (s, 9H), 1.01-0.94 (m, 1H), 0.90-0.82 (m, 1H), 0.71 (br s, 2H).

### Step 6: tert-butylN-[(7R)-5-(5-amino-1-tert-butyl-indazol-4-yl)-5-azaspiro[2.4]heptan-7-yl]carbamate

To a solution of tert-butyl N-[(7R)-5-(1-tert-butyl-5-nitro-indazol-4-yl)-5-azaspiro[2.4]heptan-7-yl]carbamate (432 mg, 1.01 mmol, 1 eq) in THF (6 mL) and H₂O (6 mL) was added Zn (550 mg, 8.41 mmol, 8.36 eq) and NH₄Cl (430.41 mg, 8.05 mmol, 8 eq). The mixture was stirred at 20° C. for 0.5 hr. LCMS showed starting material was consumed and desired mass was detected. The mixture was filtered and the filtrate was added to H₂O 15 mL and extracted with EtOAc (15 mL×3). The combined organic layers were washed with brine, dried over Na₂SO₄, filtered and concentrated under reduced pressure to give a residue. The residue was used next step directly without further purification. Compound tert-butyl N-[(7R)-5-(5-amino-1-tert-butyl-indazol-4-yl)-5-azaspiro[2.4]heptan-7-yl]carbamate (400 mg, Yield: 99.54%) was obtained as brown solid.

LC-MS m/z: 400.3 (M+1)+.

### Step 7: tert-butylN-[(7R)-5-[1-tert-butyl-5-[[1-(2,6-difluorophenyl)-6-oxo-pyridazine-3-carbonyl]amino]indazol-4-yl]-5-azaspiro[2.4]heptan-7-yl]carbamate

To a solution of 1-(2,6-difluorophenyl)-6-oxo-pyridazine-3-carboxylic acid (277.72 mg, 1.10 mmol, 1.1 eq) and tert-butyl N-[(7R)-5-(5-amino-1-tert-butyl-indazol-4-yl)-5-azaspiro [2.4]heptan-7-yl]carbamate (400 mg, 1.00 mmol, 1 eq) in MeCN (10 mL) was added NMI (246.60 mg, 3.00 mmol, 239.42 uL, 3 eq) and TCFH (309.00 mg, 1.10 mmol, 1.1 eq). The mixture was stirred at 20° C. for 1 hr. LCMS showed starting material was consumed and desired mass was detected. The reaction mixture was added to H₂O 20 mL and extracted with EtOAc (20 mL×3). The combined organic layers were washed with brine, dried over Na₂SO₄, filtered and concentrated under reduced pressure to give a residue. The residue was purified by flash silica gel chromatography (ISCO®; 12 g SepaFlash® Silica Flash Column, Eluent of 0^{˜}30% Ethyl acetate/Petroleum ethergradient). The crude product was purified by recrystallization from EtOAc (5 mL). tert-butyl N-[(7R)-5-[1-tert-butyl-5-[[1-(2,6-difluorophenyl)-6-oxo-pyridazine-3-carbonyl]amino]indazol-4-yl]-5-azaspiro[2.4]heptan-7-yl]carbamate(266 mg, Yield: 41.93%) was obtained as a yellow solid.

LC-MS m/z: 634.4 (M+1)⁺.

### Step 8: N-[4-[(7R)-7-amino-5-azaspiro[2.4]heptan-5-yl]-1-tert-butyl-indazol-5-yl]-1-(2,6-difluoro phenyl)-6-oxo-pyridazine-3-carboxamide

To a solution of tert-butyl N-[(7R)-5-[1-tert-butyl-5-[[1-(2,6-difluorophenyl)-6-oxo-pyridazine-3-carbonyl]amino]indazol-4-yl]-5-azaspiro[2.4]heptan-7-yl]carbamate (266 mg, 419.77 umol, 1 eq) in dioxane (1 mL) was added HCl/dioxane (4 M, 3 mL, 28.59 eq). The mixture was stirred at 20° C. for 1 hr. LCMS showed starting material was consumed and desired mass was detected. The mixture was concentrated under reduced pressure. Example 302 (222.88 mg, Yield: 93.15%) was obtained as a yellow solid.

LC-MS m/z: 534.3 (M+1)⁺.

¹H NMR (400 MHz, CD₃OD) δ 8.25 (d, J=9.78 Hz, 1H), 8.19 (s, 1H), 7.76 (d, J=9.18 Hz, 1H), 7.64-7.73 (m, 1H), 7.56 (d, J=9.30 Hz, 1H), 7.28-7.36 (m, 3H), 4.19 (dd, J=5.72, 10.61 Hz, 1H), 3.99 (d, J=9.18 Hz, 1H), 3.66 (dd, J=2.92, 10.55 Hz, 1H), 3.38-3.40 (m, 1H), 3.10 (d, J=9.18 Hz, 1H), 1.78 (s, 9H), 0.94-1.07 (m, 1H), 0.79-0.86 (m, 1H), 0.71-0.77 (m, 1H), 0.61-0.70 (m, 1H).

| No. | Structure | Salt form | MS and ¹HNMR |
|---|---|---|---|
| Example 303 | 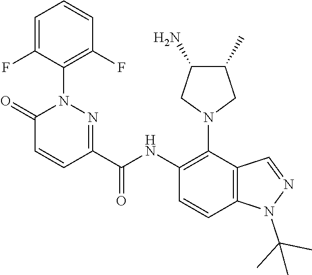 [SMILES: C[C@@H]1CN(c2c(NC(=O)c3ccc(=O)n(-c4c(F)cccc4F)n3)ccc3c2cnn3C(C)(C)C)C[C@@H]1N] N-(4-((3R,4R)-3-amino-4- methylpyrrolidin-1-yl)-1- (tert-butyl)-1H-indazol-5- yl)-1-(2,6-difluorophenyl)- 6-oxo-1,6- dihydropyridazine-3- carboxamide | HCl Salt | LC-MS m/z: 522.2(M + 1)⁺ ¹H NMR (500 MHz, CD₃OD) δ 8.23 (d, J = 9.92 Hz, 1H), 8.16 (s, 1H), 7.66-7.73 (m, 2H), 7.51 (d, J = 9.16 Hz, 1H), 7.28- 7.36 (m, 3H), 4.05 (dd, J = 6.18, 10.60 Hz, 1H), 3.82-3.89 (m, 1H), 3.62-3.68 (m, 1H), 3.55 (dd, J = 3.81, 10.53 Hz, 1H), 3.42 (t, J = 8.85 Hz, 1H), 2.53-2.65 (m, 1H), 1.75-1.80 (m, 9H), 1.14 (d, J = 7.02 Hz, 3H). |

### Example 304: N-(4-((3R,4R)-3-amino-4-methylpyrrolidin-1-yl)-2-(bicyclo[2.2.2]octan-1-yl)-2H-indazol-5-yl)-1-(2,6-difluorophenyl)-6-oxo-1,6-dihydropyridazine-3-carboxamide

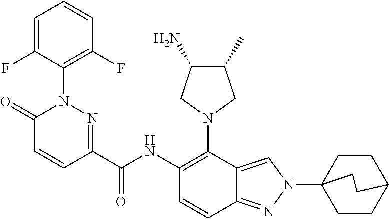 [SMILES: C[C@@H]1CN(c2c(NC(=O)c3ccc(=O)n(-c4c(F)cccc4F)n3)ccc3nn(C45CCC(CC4)CC5)cc23)C[C@@H]1N]

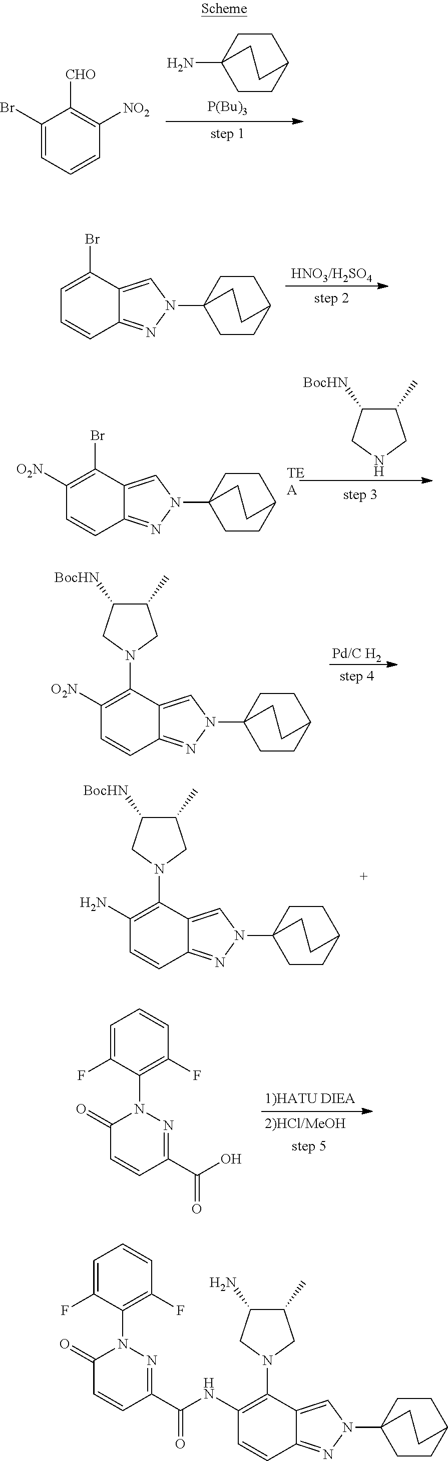

### Step 1: 2-(bicyclo[2.2.2]octan-1-yl)-4-bromo-2H-indazole

A mixture of 6-bromo-2-nitrobenzene-1-carbaldehyde (345 mg, 1.5 mmol) and bicyclo[2.2.2]octan-1-amine chlorane (266.76 mg, 1.65 mmol) in dioxane (10 mL), was stirred for 0.5 h at 90° C. as a black solution, then was added tributyl phosphine (910.44 mg, 4.500 mmol) and was stirred for 24 h at 90° C. as a black solution. The mixture was poured into water 30 mL, extracted with EA (30 mL×2). The mixture of reaction was concentrated to dryness in vacuo. The resulting residue was purified by column (SiO₂, eluted with 20% EA in PE, COMBI FLASH NEXTAGEN 300) to afford 2-(bicyclo[2.2.2]octan-1-yl)-4-bromoindazole (260 mg, Yield: 56.79%) as a white solid.

LC-MS m/z: 306.2 (M+1)⁺.

### Step2: 2-(bicyclo[2.2.2]octan-1-yl)-4-bromo-5-nitro-2H-indazole

To a solution of 2-(bicyclo[2.2.2]octan-1-yl)-4-bromoindazole (150 mg, 0.491 mmol) in H₂SO₄ (5 mL, 93.811 mmol), was added Fuming Nitric Acid (0.25 mL, 0.491 mmol) at 0° C., and was stirred at 0° C., for 30 min as a brown solution, Target compound MS was detected by LC-MS. The reaction was diluted with H₂O 30 mL at 0° C., and extracted with EA30ML*2. The combined organic layers was washed by NaHCO₃ and were dried over Na₂SO₄, filtered and concentrated to afford 2-(bicyclo[2.2.2]octan-1-yl)-4-bromo-5-nitroindazole (162 mg, Yield: 94.12%) as a yellow solid which was directly used to next reaction.

LC-MS m/z: 351.2 (M+1)⁺.

### Step 3: tert-butyl ((3R,4R)-1-(2-(bicyclo[2.2.2]octan-1-yl)-5-nitro-2H-indazol-4-yl)-4-methylpyrrolidin-3-yl)carbamate

To a solution of 2-(bicyclo[2.2.2]octan-1-yl)-4-bromo-5-nitroindazole (162 mg, 0.463 mmol) in dioxane (5 mL), were added 2-methylpropan-2-yl {[(3R,4R)-4-methyltetrahydro-1H-pyrrol-3-yl]amino}methanoate (102.00 mg, 0.509 mmol) and TEA (0.5 mL, 3.597 mmol). The mixture of reaction was stirred at 110° C. for 5 hr as a brown suspension. LC-MS showed the reaction finished. The reaction was diluted with H₂O 100 mL and extracted with EA (50 ML×2). The combined organic layers were dried over Na₂SO₄, filtered and concentrated to afford 2-methylpropan-2-yl{[(3R,4R)-1-[2-(bicyclo[2.2.2]octan-1-yl)-5-nitroindazol-4-yl]-4-methyltetrahydro-1H-pyrrol-3-yl]amino}methanoate (210 mg, Yield: 96.68%) as a yellow solid.

LC-MS m/z: 470.6 (M+1)⁺.

### Step 4: tert-butyl ((3R,4R)-1-(5-amino-2-(bicyclo[2.2.2]octan-1-yl)-2H-indazol-4-yl)-4-methylpyrrolidin-3-yl)carbamate

To a solution of 2-methylpropan-2-yl {[(3R,4R)-1-[2-(bicyclo[2.2.2]octan-1-yl)-5-nitroindazol-4-yl]-4-methyltetrahydro-1H-pyrrol-3-yl]amino}methanoate (210 mg, 0.447 mmol) in MeOH (10 mL) was added Pd/C 10% (20 mg, 0.188 mmol) and under hydrogen for 18 h at RT (60 Psi) as a black suspension, Target compound MS was detected by LC-MS. The mixture of reaction was filtered, the filtrate was concentrated to dryness in vacuo affording 2-methylpropan-2-yl{[(3R,4R)-1-[5-amino-2-(bicyclo[2.2.2]octan-1-yl)indazol-4-yl]-4-methyltetrahydro-1H-pyrrol-3-yl]amino}methanoate (182 mg, Yield: 92.58%) as a black solid which was directly used to the next reaction.

LC-MS m/z: 440.6 (M+1)⁺.

### Step 5: N-(4-((3R,4R)-3-amino-4-methylpyrrolidin-1-yl)-2-(bicyclo[2.2.2]octan-1-yl)-2H-indazol-5-yl)-1-(2,6-difluorophenyl)-6-oxo-1,6-dihydropyridazine-3-carboxamide

To a solution of 1-(2,6-difluorophenyl)-6-oxo-1,2-diazine-3-carboxylic acid (100 mg, 0.397 mmol) 2-methylpropan-2-yl{[(3R,4R)-1-[5-amino-2-(bicyclo[2.2.2]octan-1-yl)indazol-4-yl]-4-methyltetrahydro-1H-pyrrol-3-yl]amino}methanoate (174.32 mg, 0.397 mmol) in ACN (10 mL) was added HATU (226.43 mg, 0.596 mmol) and Ethyldiisopropylamine (0.5 mL, 3.025 mmol), was stirred for 2 h at r.t to get a black suspension. LC-MS showed most of the start material was consumed and one peak was detected. The reaction was diluted with H₂O 30 mL and extracted with EA30 mL×2. The combined organic layers were dried over Na₂SO₄, filtered and concentrated. The resulting residue was purified by column (SiO₂, eluted with 60% EA in PE, COMBI FLASH NEXTAGEN 300) to afford 68 mg as a yellow solid. The residue was added 15 mL HCl/EtOH (4 mol/L), and stirred at 50° C. for 3 h as a yellow suspension, filtered and dried to afford Example 304 (44.76 mg, Yield: 18.48%) as a yellow solid.

LC-MS m/z: 574.6 (M+1)⁺.

¹H NMR (400 MHz, MeOD) δ 8.38 (s, 1H), 8.22 (d, J=9.9 Hz, 1H), 7.73-7.62 (m, 1H), 7.57 (d, J=9.1 Hz, 1H), 7.36-7.22 (m, 4H), 3.99 (dd, J=10.6, 6.1 Hz, 1H), 3.82 (dd, J=9.8, 6.3 Hz, 1H), 3.60 (dd, J=9.2, 7.6 Hz, 1H), 3.54-3.44 (m, 1H), 3.42-3.35 (m, 1H), 2.83 (s, 1H), 2.59 (dt, J=14.8, 7.3 Hz, 1H), 2.23 (dd, J=9.8, 6.2 Hz, 6H), 1.91 (dd, J=9.4, 6.2 Hz, 6H), 1.81 (dd, J=6.1, 3.0 Hz, 1H), 1.15 (d, J=7.1 Hz, 3H).

| No. | Structure | Salt form | MS and 1HNMR |
|---|---|---|---|
| Example 305 | 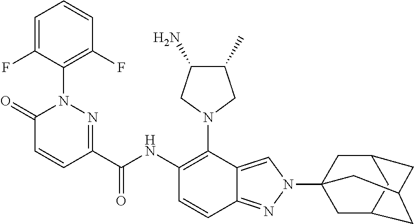 [SMILES: C[C@@H]1CN(c2c(NC(=O)c3ccc(=O)n(-c4c(F)cccc4F)n3)ccc3nn(C45CC6CC(CC(C6)C4)C5)cc23)C[C@@H]1N] N-(2-((3S,5S,7S)- adamantan-1-yl)-4- ((3R,4R)-3-amino-4- methylpyrrolidin-1-yl)-2H- indazol-5-yl)-1-(2,6- difluorophenyl)-6-oxo-1,6- dihydropyridazine-3- carboxamide | HCl Salt | LC-MS m/z: 600.3 (M + 1)⁺. ¹H NMR (400 MHz, MeOD) δ 8.43 (s, 1H), 8.23 (d, J = 9.8 Hz, 1H), 7.76-7.61 (m, 2H), 7.39-7.23 (m, 4H), 3.86 (dd, J = 9.9, 6.0 Hz, 1H), 3.58 (d, J = 4.1 Hz, 1H), 3.49 (dd, J = 11.0, 5.3 Hz, 1H), 2.39 (dd, J = 18.1, 5.0 Hz, 6H), 2.30 (s, 3H), 1.89 (s, 6H), 1.06 (d, J = 7.1 Hz, 3H). |
| Example 306 | 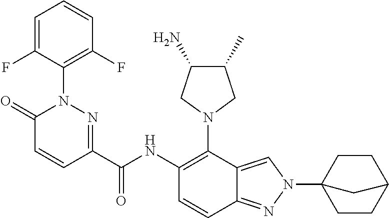 [SMILES: C[C@@H]1CN(c2c(NC(=O)c3ccc(=O)n(-c4c(F)cccc4F)n3)ccc3nn(C45CCC(CC4)C5)cc23)C[C@@H]1N] N- (4-((3R,4R)-3-amino-4- methylpyrrolidin-1-yl)-2- (bicyclo[2.2.1]heptan-1- yl)-2H-indazol-5-yl)-1-(2,6- difluorophenyl)-6-oxo-1,6- dihydropyridazine-3- carboxamide | HCl Salt | LC-MS m/z: 560.6 (M + 1)⁺. ¹H NMR (400 MHz, MeOD) δ 8.41 (s, 1H), 8.22 (d, J = 9.9 Hz, 1H), 7.69 (td, J = 8.6, 4.3 Hz, 1H), 7.60 (d, J = 9.1 Hz, 1H), 7.30 (dd, J = 16.6, 9.4 Hz, 4H), 3.98 (dd, J = 10.5, 6.0 Hz, 1H), 3.78 (dd, J = 9.9, 6.2 Hz, 1H), 3.60 (dd, J = 9.2, 7.6 Hz, 1H), 3.52- 3.42 (m, 1H), 2.57 (dd, J = 14.2, 7.4 Hz, 1H), 2.46 (s, 1H), 2.24-1.97 (m, 8H), 1.69 (d, J = 8.5 Hz, 2H), 1.13 (d, J = 7.1 Hz, 3H). |
| Example 307 | 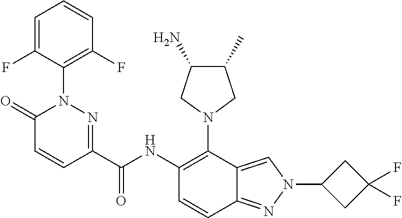 [SMILES: C[C@@H]1CN(c2c(NC(=O)c3ccc(=O)n(-c4c(F)cccc4F)n3)ccc3nn(C4CC(F)(F)C4)cc23)C[C@@H]1N] N-(4-((3R,4R)-3-amino-4- methylpyrrolidin-1-yl)-2- (3,3-difluorocyclobutyl)- 2H-indazol-5-yl)-1-(2,6- difluorophenyl)-6-oxo- 1,6-dihydropyridazine-3- carboxamide | HCl Salt | LC-MS m/z: 556.5 (M + 1)⁺. ¹H NMR (400 MHz, MeOD) δ 8.55 (s, 1H), 8.48 (s, 1H), 8.22 (d, J = 9.8 Hz, 1H), 7.68 (tt, J = 8.6, 6.3 Hz, 1H), 7.54 (d, J = 9.1 Hz, 1H), 7.38-7.15 (m, 4H), 5.14 (dd, J = 12.8, 7.6 Hz, 1H), 3.99 (dd, J = 10.5, 5.9 Hz, 1H), 3.81-3.75 (m, 1H), 3.63 (dd, J = 9.2, 7.6 Hz, 1H), 3.51-3.35 (m, 4H), 2.83 (s, 3H), 2.63-2.46 (m, 1H), 1.14 (d, J = 7.1 Hz, 3H). |
| Example 308 | 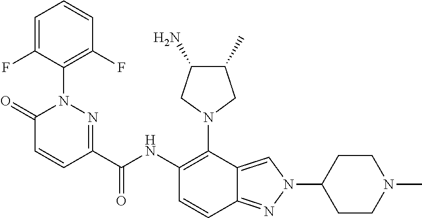 [SMILES: C[C@@H]1CN(c2c(NC(=O)c3ccc(=O)n(-c4c(F)cccc4F)n3)ccc3nn(C4CCN(C)CC4)cc23)C[C@@H]1N] N-(4-((3R,4R)-3-amino-4- methylpyrrolidin-1-yl)-2- (1-methylpiperidin-4-yl)- 2H-indazol-5-yl)-1-(2,6- difluorophenyl)-6-oxo- 1,6-dihydropyridazine-3- carboxamide | HCl Salt | LC-MS m/z: 563.6(M + 1)⁺. ¹H NMR (400 MHz, MeOD) δ 8.53 (s, 1H), 8.40-8.29 (m, 1H), 8.22 (d, J = 9.9 Hz, 1H), 7.69 (d, J = 6.2 Hz, 1H), 7.54 (d, J = 9.1 Hz, 1H), 7.32 (d, J = 9.9 Hz, 3H), 7.25 (d, J = 8.9 Hz, 1H), 4.05-3.85 (m, 1H), 3.81 (s, 1H), 3.67-3.56 (m, 1H), 3.52- 3.39 (m, 5H), 2.87 (s, 2H), 2.73 (s, 3H), 2.61 (d, J = 7.6 Hz, 1H), 2.38 (d, J = 25.7 Hz, 4H), 1.38-1.22 (m, 3H), 1.15 (d, J = 7.1 Hz, 3H), 0.94-0.76 (m, 1H). |
| Example 309 | 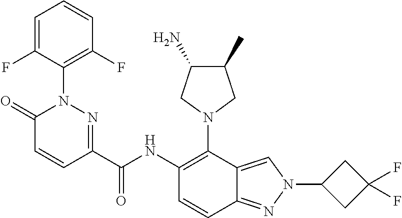 [SMILES: C[C@H]1CN(c2c(NC(=O)c3ccc(=O)n(-c4c(F)cccc4F)n3)ccc3nn(C4CC(F)(F)C4)cc23)C[C@@H]1N] N-(4-((3R,4S)-3-amino-4- methylpyrrolidin-1-yl)-2- (3,3-difluorocyclobutyl)- 2H-indazol-5-yl)-1-(2,6- difluorophenyl)-6-oxo- 1,6-dihydropyridazine-3- carboxamide | HCl Salt | LC-MS m/z: 556.5(M + 1)⁺. ¹H NMR (400 MHz, MeOD) δ 8.54 (d, J = 10.5 Hz, 1H), 8.24 (d, J = 9.8 Hz, 1H), 7.82 (d, J = 9.2 Hz, 1H), 7.67 (td, J = 8.6, 4.3 Hz, 1H), 7.44-7.21 (m, 4H), 3.87-3.62 (m, 2H), 3.29-3.21 (m, 2H), 3.06 (dd, J = 9.3, 5.5 Hz, 1H), 2.34-2.11 (m, 1H), 1.01 (t, J = 35.5 Hz, 3H). |
| Example 310 | 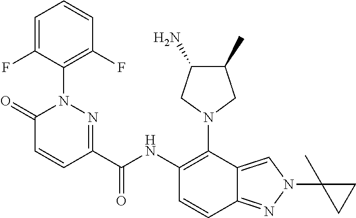 [SMILES: C[C@H]1CN(c2c(NC(=O)c3ccc(=O)n(-c4c(F)cccc4F)n3)ccc3nn(C4(C)CC4)cc23)C[C@@H]1N] N-(4-((3R,4S)-3-amino-4- methylpyrrolidin-1-yl)-2- (1-methylcyclopropyl)-2H- indazol-5-yl)-1-(2,6- difluorophenyl)-6-oxo- 1,6-dihydropyridazine-3- carboxamide | HCl Salt | LC-MS m/z: 520.6(M + 1)⁺. ¹H NMR (400 MHz, MeOD) δ 8.52 (s, 1H), 8.24 (d, J = 9.8 Hz, 1H), 7.89 (dd, J = 20.6, 9.3 Hz, 1H), 7.66 (dd, J = 8.5, 6.3 Hz, 1H), 7.31 (dt, J = 12.1, 7.5 Hz, 4H), 3.73 (dd, J = 16.4, 7.1 Hz, 2H), 3.55 (d, J = 36.5 Hz, 1H), 3.13-2.98 (m, 2H), 2.10 (s, 1H), 1.75 (s, 3H), 1.43 (t, J = 5.9 Hz, 2H), 1.31 (s, 1H), 1.19-1.08 (m, 3H), 1.02 (d, J = 6.8 Hz, 3H), 0.90 (d, J = 6.7 Hz, 1H). |
| Example 311 | 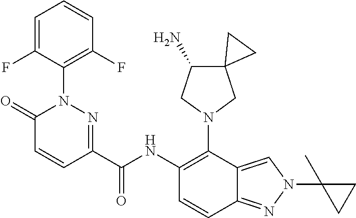 [SMILES: CC1(n2cc3c(N4C[C@H](N)C5(CC5)C4)c(NC(=O)c4ccc(=O)n(-c5c(F)cccc5F)n4)ccc3n2)CC1] (R)-N-(4-(7-amino-5- azaspiro[2.4]heptan-5-yl)- 2-(1-methylcyclopropyl)- 2H-indazol-5-yl)-1-(2,6- difluorophenyl)-6-oxo- 1,6-dihydropyridazine-3- carboxamide | HCl Salt | LC-MS m/z: 532.6(M + 1)⁺. ¹H NMR (400 MHz, MeOD) δ 8.53 (d, J = 1.4 Hz, 1H), 8.23 (d, J = 9.8 Hz, 1H), 7.82- 7.56 (m, 1H), 7.37-7.04 (m, 4H), 4.11 (dd, J = 10.3, 5.5 Hz, 1H), 3.91 (d, J = 9.1 Hz, 1H), 3.59-3.44 (m, 1H), 3.06 (d, J = 9.1 Hz, 1H), 1.76 (s, 3H), 1.52-1.36 (m, 1H), 1.22-1.03 (m, 2H), 1.02-0.86 (m, 1H), 0.79-0.61 (m, 3H). |
| Example 312 | 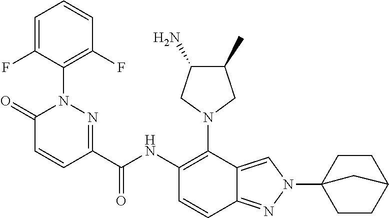 [SMILES: C[C@H]1CN(c2c(NC(=O)c3ccc(=O)n(-c4c(F)cccc4F)n3)ccc3nn(C45CCC(CC4)C5)cc23)C[C@@H]1N] N-(4-((3R,4S)-3-amino-4- methylpyrrolidin-1-yl)-2- (bicyclo[2.2.1]heptan-1- yl)-2H-indazol-5-yl)-1-(2,6- difluorophenyl)-6-oxo- 1,6-dihydropyridazine-3- carboxamide | HCl Salt | LC-MS m/z: 560.6(M + 1)⁺. ¹H NMR (400 MHz, MeOD) δ 8.44 (s, 1H), 8.25 (d, J = 9.9 Hz, 1H), 7.87 (d, J = 9.2 Hz, 1H), 7.68 (td, J = 8.6, 4.3 Hz, 1H), 7.34 (dt, J = 15.2, 10.1 Hz, 4H), 3.85 (dd, J = 9.8, 6.9 Hz, 2H), 3.43 (dd, J = 10.1, 5.5 Hz, 1H), 3.11-3.00 (m, 1H), 2.83 (s, 1H), 2.47 (s, 1H), 2.34 (s, 1H), 2.22-1.98 (m, 8H), 1.69 (d, J = 9.0 Hz, 2H), 1.47-1.36 (m, 1H), 1.08 (d, J = 7.0 Hz, 3H). |
| Example 313 | 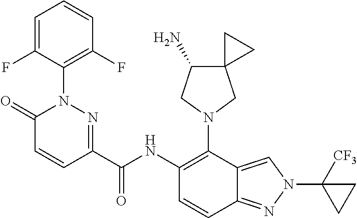 [SMILES: N[C@H]1CN(c2c(NC(=O)c3ccc(=O)n(-c4c(F)cccc4F)n3)ccc3nn(C4(C(F)(F)F)CC4)cc23)CC12CC2] rel-(R)-N-(4-(7-amino-5- azaspiro[2.4]heptan-5-yl)- 2-(1- (trifluoromethyl)cyclopropyl)- 2H-indazol-5-yl)-1- (2,6-difluorophenyl)-6- oxo-1,6- dihydropyridazine-3- carboxamide | Free base | LC-MS m/z: 586.3(M + 1)⁺. ¹H NMR (400 MHz, CD₃OD) δ 8.76 (s, 1H), 8.23 (d, J = 9.9 Hz, 1H), 7.76-7.65 (m, 1H), 7.60 (d, J = 9.2 Hz, 1H), 7.38-7.23 (m, 4H), 4.21 (dd, J = 5.5, 10.7 Hz, 1H), 4.03 (d, J = 9.2 Hz, 1H), 3.72-3.62 (m, 1H), 3.40 (dd, J = 2.1, 5.4 Hz, 1H), 3.10 (d, J = 9.2 Hz, 1H), 1.80 (s, 4H), 1.04 (td, J = 5.9, 9.7 Hz, 1H), 0.93-0.82 (m, 1H), 0.77-0.61 (m, 2H). |
| Example 314 | 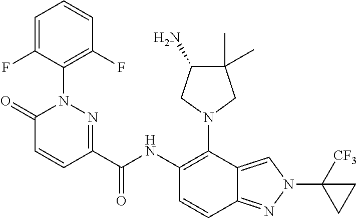 [SMILES: CC1(C)CN(c2c(NC(=O)c3ccc(=O)n(-c4c(F)cccc4F)n3)ccc3nn(C4(C(F)(F)F)CC4)cc23)C[C@@H]1N] rel-(R)-N-(4-(4-amino-3,3- dimethylpyrrolidin-1-yl)- 2-(1- (trifluoromethyl)cyclopropyl)- 2H-indazol-5-yl)-1- (2,6-difluorophenyl)-6- oxo-1,6- dihydropyridazine-3- carboxamide | Free base | LC-MS m/z: 588.2(M + 1)⁺. ¹H NMR (400 MHz, CD₃OD) δ 8.61 (s, 1H), 8.12 (d, J = 9.9 Hz, 1H), 7.62-7.50 (m, 2H), 7.24-7.08 (m, 4H), 3.92 (dd, J = 6.8, 10.4 Hz, 1H), 3.46 (d, J = 9.1 Hz, 1H), 3.39- 3.33 (m, 1H), 3.33-3.28 (m, 1H), 3.25 (d, J = 9.2 Hz, 1H), 1.68 (s, 4H), 1.05 (d, J = 10.6 Hz, 6H). |
| Example 315 | 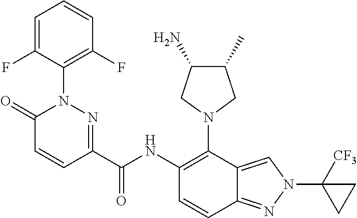 [SMILES: C[C@@H]1CN(c2c(NC(=O)c3ccc(=O)n(-c4c(F)cccc4F)n3)ccc3nn(C4(C(F)(F)F)CC4)cc23)C[C@@H]1N] N-(4-((3R,4R)-3-amino-4- methylpyrrolidin-1-yl)-2- (1- (trifluoromethyl)cyclopropyl)- 2H-indazol-5-yl)-1- (2,6-difluorophenyl)-6- oxo-1,6- dihydropyridazine-3- carboxamide | HCl Salt | LC-MS m/z: 574.2(M + 1)⁺. 1H NMR (400 MHz, CD₃OD) δ 8.73 (s, 1H), 8.26-8.16 (m, 1H), 7.74-7.62 (m, 1H), 7.51 (d, J = 9.2 Hz, 1H), 7.37-7.19 (m, 4H), 4.04 (dd, J = 5.9, 10.8 Hz, 1H), 3.90-3.80 (m, 1H), 3.68 (dd, J = 7.8, 9.1 Hz, 1H), 3.58-3.42 (m, 2H), 2.71-2.56 (m, 1H), 1.86-1.72 (m, 4H), 1.17 (d, J = 7.2 Hz, 3H). |
| Example 316 | 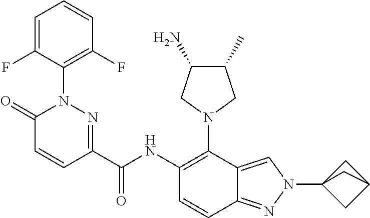 [SMILES: C[C@@H]1CN(c2c(NC(=O)c3ccc(=O)n(-c4c(F)cccc4F)n3)ccc3nn(C45CC(C4)C5)cc23)C[C@@H]1N] N-(4-((3R,4R)-3-amino-4- methylpyrrolidin-1-yl)-2- (bicyclo[1.1.1]pentan-1- yl)-2H-indazol-5-yl)-1-(2,6- difluorophenyl)-6-oxo- 1,6-dihydropyridazine-3- carboxamide | HCOOH salt | LC-MS m/z: 532.3 (M + 1)⁺ ¹H NMR (400 MHz, CD₃OD) δ 8.38 (d, J = 1.0 Hz, 1H), 8.20 (d, J = 9.8 Hz, 1H), 7.71- 7.60 (m, 2H), 7.34-7.25 (m, 3H), 7.22 (dd, J = 9.1, 1.0 Hz, 1H), 3.87 (dd, J = 10.0, 6.0 Hz, 1H), 3.59 (td, J = 5.9, 3.6 Hz, 1H), 3.50 (dd, J = 9.1, 7.3 Hz, 1H), 3.38-3.32 (m, 2H), 2.72 (s, 1H), 2.44 (s, 6H), 2.42- 2.35 (m, 1H), 1.05 (d, J = 7.0 Hz, 3H). |
| Example 317 | 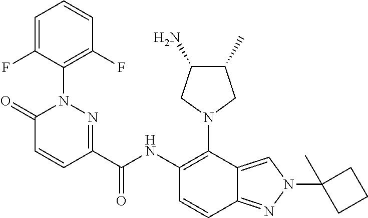 [SMILES: C[C@@H]1CN(c2c(NC(=O)c3ccc(=O)n(-c4c(F)cccc4F)n3)ccc3nn(C4(C)CCC4)cc23)C[C@@H]1N] 1-(2,6-difluorophenyl)-N- {4-[(3R,4R)-3-amino-4- methyltetrahydro-1H- pyrrol-1-yl]-2- (methylcyclobutyl)indazol- 5-yl}-6-oxo-1,2-diazine-3- carboxamide | HCOOHSalt | LC-MS m/z: 534.3 (M + 1)⁺ ¹H NMR (400 MHz, CD₃OD) δ 8.53 (s, 1H), 8.37 (s, 1H), 8.19 (d, J = 9.8 Hz, 1H), 7.71-7.60 (m, 2H), 7.34-7.21 (m, 4H), 3.87 (dd, J = 10.1, 6.1 Hz, 1H), 3.63 (td, J = 6.1, 3.7 Hz, 1H), 3.48 (dd, J = 9.1, 7.3 Hz, 1H), 3.36 (dd, J = 10.3, 3.7 Hz, 1H), 2.83 (qd, J = 9.7, 2.7 Hz, 2H), 2.47-2.34 (m, 3H), 2.10 (dq, J = 11.5, 9.0 Hz, 1H), 2.03-1.92 (m, 1H), 1.80 (s, 3H), 1.06 (d, J = 7.1 Hz, 3H). |
| Example 318 | 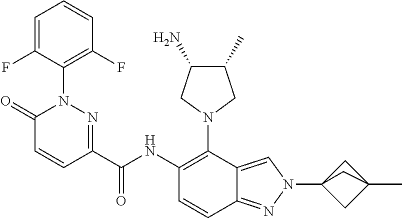 [SMILES: C[C@@H]1CN(c2c(NC(=O)c3ccc(=O)n(-c4c(F)cccc4F)n3)ccc3nn(C45CC(C)(C4)C5)cc23)C[C@@H]1N] N-(4-((3R,4R)-3-amino-4- methylpyrrolidin-1-yl)-2- (3- methylbicyclo[1.1.1]pentan- 1-yl)-2H-indazol-5-yl)-1- (2,6-difluorophenyl)-6- oxo-1,6- dihydropyridazine-3- carboxamide | HCOOH salt | LC-MS m/z: 546.3 (M + 1)⁺ ¹H NMR (400 MHz, CD₃OD) δ 8.35 (s, 1H), 8.20 (d, J = 9.8 Hz, 1H), 7.71-7.63 (m, 1H), 7.61 (d, J = 9.2 Hz, 1H), 7.29 (dt, J = 9.1, 6.0 Hz, 3H), 7.23 (d, J = 9.1 Hz, 1H), 3.93 (dd, J = 10.4, 6.0 Hz, 1H), 3.73 (td, J = 6.1, 3.5 Hz, 1H), 3.54 (dd, J = 9.2, 7.4 Hz, 1H), 3.42 (dd, J = 10.4, 3.6 Hz, 1H), 3.36 (t, J = 8.7 Hz, 1H), 2.49 (h, J = 7.3 Hz, 1H), 2.29 (s, 6H), 1.41 (s, 3H), 1.10 (d, J = 7.1 Hz, 3H). |
| Example 319 | 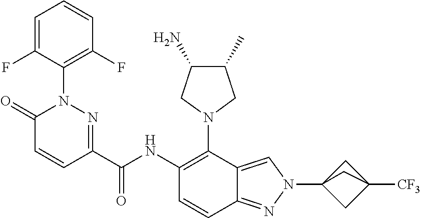 [SMILES: C[C@@H]1CN(c2c(NC(=O)c3ccc(=O)n(-c4c(F)cccc4F)n3)ccc3nn(C45CC(C(F)(F)F)(C4)C5)cc23)C[C@@H]1N] N-(4-((3R,4R)-3-amino-4- methylpyrrolidin-1-yl)-2- (3- (trifluoromethyl)bicyclo [1.1.1]pentan-1-yl)-2H- indazol-5-yl)-1-(2,6- difluorophenyl)-6-oxo-1,6- dihydropyridazine-3- carboxamide | HCOOH salt | LC-MS m/z: 600.3 (M + 1)⁺ ¹H NMR (400 MHz, CD₃OD) δ 8.51 (d, J = 8.5 Hz, 1H), 8.19 (d, J = 9.9 Hz, 1H), 7.71- 7.58 (m, 2H), 7.35-7.24 (m, 3H), 7.21 (d, J = 9.1 Hz, 1H), 3.89 (dd, J = 10.1, 5.9 Hz, 1H), 3.62 (td, J = 6.0, 3.6 Hz, 1H), 3.52 (dd, J = 9.1, 7.4 Hz, 1H), 3.42-3.33 (m, 2H), 2.69 (s, 6H), 2.44 (hept, J = 7.1 Hz, 1H), 1.07 (d, J = 7.0 Hz, 3H). |
| Example 320 | 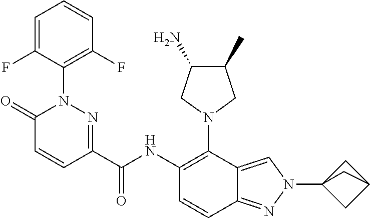 [SMILES: C[C@H]1CN(c2c(NC(=O)c3ccc(=O)n(-c4c(F)cccc4F)n3)ccc3nn(C45CC(C4)C5)cc23)C[C@@H]1N] N-(4-((3R,4S)-3-amino-4- methylpyrrolidin-1-yl)-2- (bicyclo[1.1.1]pentan-1- yl)-2H-indazol-5-yl)-1-(2,6- difluorophenyl)-6-oxo-1,6- dihydropyridazine-3- carboxamide | HCOOH salt | LC-MS m/z: 532.3 (M + 1)⁺ ¹H NMR (400 MHz, CD₃OD) δ 8.40 (d, J = 1.0 Hz, 1H), 8.21 (d, J = 9.8 Hz, 1H), 7.88 (d, J = 9.1 Hz, 1H), 7.66 (tt, J = 8.5, 6.2 Hz, 1H), 7.36-7.21 (m, 4H), 3.76 (ddd, J = 9.9, 7.1, 5.4 Hz, 2H), 3.35 (dd, J = 6.2, 3.6 Hz, 1H), 3.19 (q, J = 6.0 Hz, 1H), 3.01 (dd, J = 9.3, 5.3 Hz, 1H), 2.73 (s, 1H), 2.45 (s, 6H), 2.19 (tt, J = 7.3, 5.5 Hz, 1H), 1.02 (d, J = 6.9 Hz, 3H). |
| Example 321 | 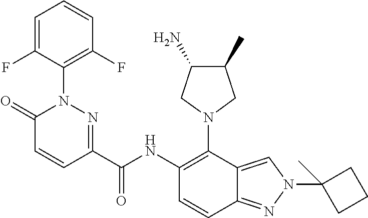 [SMILES: C[C@H]1CN(c2c(NC(=O)c3ccc(=O)n(-c4c(F)cccc4F)n3)ccc3nn(C4(C)CCC4)cc23)C[C@@H]1N] N-(4-((3R,4S)-3-amino-4- methylpyrrolidin-1-yl)-2- (1-methylcyclobutyl)-2H- indazol-5-yl)-1-(2,6- difluorophenyl)-6-oxo-1,6- dihydropyridazine-3- carboxamide | HCOOH salt | LC-MS m/z: 534.3 (M + 1)⁺ ¹H NMR (400 MHz, CD₃OD) δ 8.54-8.45 (m, 1H), 8.22 (d, J = 9.9 Hz, 1H), 7.91 (d, J = 9.2 Hz, 1H), 7.67 (tt, J = 8.6, 6.2 Hz, 1H), 7.41-7.35 (m, 1H), 7.29 (dd, J = 9.7, 7.9 Hz, 3H), 3.84 (ddd, J = 15.2, 9.8, 7.1 Hz, 2H), 3.46 (dd, J = 10.2, 5.8 Hz, 1H), 3.02 (dd, J = 9.4, 4.6 Hz, 1H), 2.86 (dt, J = 12.5, 9.7 Hz, 2H), 2.47-2.31 (m, 3H), 2.11 (dq, J = 11.4, 9.0 Hz, 1H), 2.04-1.93 (m, 1H), 1.82 (s, 3H), 1.05 (d, J = 6.9 Hz, 3H). |
| Example 322 | 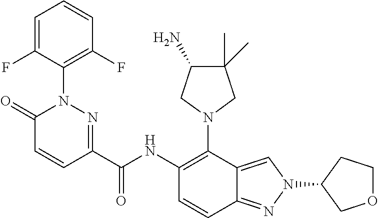 [SMILES: CC1(C)CN(c2c(NC(=O)c3ccc(=O)n(-c4c(F)cccc4F)n3)ccc3nn([C@@H]4CCOC4)cc23)C[C@@H]1N] N-(4-((R)-4-amino-3,3- dimethylpyrrolidin-1-yl)-2- ((R)-tetrahydrofuran-3-yl)- 2H-indazol-5-yl)-1-(2,6- difluorophenyl)-6-oxo-1,6- dihydropyridazine-3- carboxamide | HCl salt | LC-MS m/z: 550.1 (M + 1)⁺. ¹H NMR (400 MHz, CD₃OD) δ 8.60-8.55 (m, 1H), 8.26-8.18 (m, 1H), 7.74-7.60 (m, 2H), 7.33-7.24 (m, 4H), 5.40-5.31 (m, 1H), 4.26-4.11 (m, 3H), 4.03-3.92 (m, 2H), 3.58-3.52 (m, 1H), 3.47-3.40 (m, 2H), 3.36-3.34 (m, 1H), 2.69-2.58 (m, 1H), 2.50-2.41 (m, 1H), 1.15 (d, J = 10.8 Hz, 6H). |
| Example 323 | 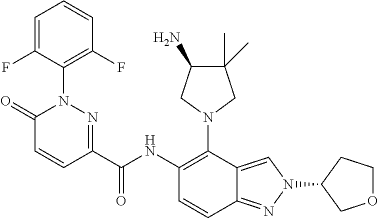 [SMILES: CC1(C)CN(c2c(NC(=O)c3ccc(=O)n(-c4c(F)cccc4F)n3)ccc3nn([C@@H]4CCOC4)cc23)C[C@H]1N] N-(4-((S)-4-amino-3,3- dimethylpyrrolidin-1-yl)-2- ((R)-tetrahydrofuran-3-yl)- 2H-indazol-5-yl)-1-(2,6- difluorophenyl)-6-oxo-1,6- dihydropyridazine-3- carboxamide | HCl salt | LC-MS m/z: 550.1(M + 1)⁺. ¹H NMR (400 MHz, CD₃OD) δ 8.60-8.57 (m, 1H), 8.26-8.19 (m, 1H), 7.73-7.61 (m, 2H), 7.33-7.23 (m, 4H), 5.40-5.30 (m, 1H), 4.26-4.11 (m, 3H), 4.03-3.92 (m, 2H), 3.57-3.53 (m, 1H), 3.48-3.39 (m, 2H), 3.36-3.34 (m, 1H), 2.71-2.57 (m, 1H), 2.51-2.38 (m, 1H), 1.18-1.12 (m, 6H). |
| Example 324 | 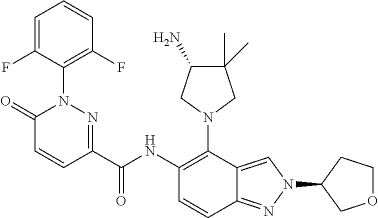 [SMILES: CC1(C)CN(c2c(NC(=O)c3ccc(=O)n(-c4c(F)cccc4F)n3)ccc3nn([C@H]4CCOC4)cc23)C[C@@H]1N] N-(4-((R)-4-amino-3,3- dimethylpyrrolidin-1-yl)-2- ((S)-tetrahydrofuran-3-yl)- 2H-indazol-5-yl)-1-(2,6- difluorophenyl)-6-oxo-1,6- dihydropyridazine-3- carboxamide | HCl Salt | LC-MS m/z: 550.1(M + 1)⁺. ¹H NMR (400 MHz, CD₃OD) δ 8.54 (s, 1H), 8.12 (d, J = 9.8 Hz, 1H), 7.63 (br d, J = 9.2 Hz, 2H), 7.19 (br t, J = 9.7 Hz, 4H), 5.28 (br s, 1H), 4.22-3.98 (m, 4H), 3.95 -3.77 (m, 2H), 3.46 (br d, J = 9.1 Hz, 1H), 3.41-3.32 (m, 2H), 2.62-2.47 (m, 1H), 2.39 (br s, 1H), 1.05 (br d, J = 16.2 Hz, 6H). |
| Example 325 | 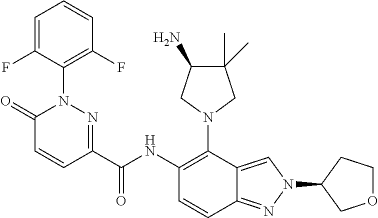 [SMILES: CC1(C)CN(c2c(NC(=O)c3ccc(=O)n(-c4c(F)cccc4F)n3)ccc3nn([C@H]4CCOC4)cc23)C[C@H]1N] N-(4-((S)-4-amino-3,3- dimethylpyrrolidin-1-yl)-2- ((S)-tetrahydrofuran-3-yl)- 2H-indazol-5-yl)-1-(2,6- difluorophenyl)-6-oxo-1,6- dihydropyridazine-3- carboxamide | HCl Salt | LC-MS m/z: 550.1(M + 1)⁺. ¹H NMR (400 MHz, CD₃OD) δ 8.65 (s, 1H), 8.24 (d, J = 9.9 Hz, 1H), 7.76-7.64 (m, 2H), 7.36-7.27 (m, 4H), 5.44-5.34 (m, 1H), 4.30-4.12 (m, 3H), 4.06-3.92 (m, 2H), 3.58 (d, J = 9.2 Hz, 1H), 3.52- 3.44 (m, 2H), 3.36 (d, J = 9.2 Hz, 1H), 2.73- 2.60 (m, 1H), 2.53-2.42 (m, 1H), 1.17 (d, J = 14.2 Hz, 6H). |
| Example 326 | 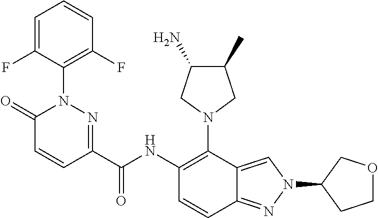 [SMILES: C[C@H]1CN(c2c(NC(=O)c3ccc(=O)n(-c4c(F)cccc4F)n3)ccc3nn([C@@H]4CCOC4)cc23)C[C@@H]1N] N-(4-((3R,4S)-3-amino-4- methylpyrrolidin-1-yl)-2- ((R)-tetrahydrofuran-3-yl)- 2H-indazol-5-yl)-1-(2,6- difluorophenyl)-6-oxo-1,6- dihydropyridazine-3- carboxamide | HCl Salt | LC-MS m/z: 536.2(M + 1)⁺. ¹H NMR (500 MHz, CD₃OD) δ 8.68 (s, 1H), 8.24 (d, J = 9.8 Hz, 1H), 7.81 (d, J = 9.2 Hz, 1H), 7.68 (tt, J = 6.2, 8.6 Hz, 1H), 7.36- 7.28 (m, 4H), 5.41 (tdd, J = 3.1, 5.8, 8.5 Hz, 1H), 4.32-4.21 (m, 2H), 4.18-4.11 (m, 1H), 4.01-3.88 (m, 3H), 3.54-3.47 (m, 1H), 3.42-3.38 (m, 1H), 3.22-3.13 (m, 1H), 2.74-2.64 (m, 1H), 2.52-2.43 (m, 1H), 2.42-2.36 (m, 1H), 1.11 (d, J = 7.0 Hz, 3H). |
| Example 327 | 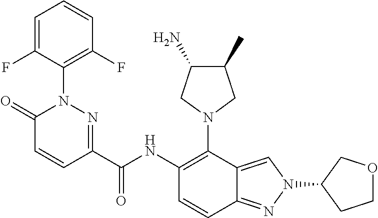 [SMILES: C[C@H]1CN(c2c(NC(=O)c3ccc(=O)n(-c4c(F)cccc4F)n3)ccc3nn([C@H]4CCOC4)cc23)C[C@@H]1N] N-(4-((3R,4S)-3-amino-4- methylpyrrolidin-1-yl)-2- ((S)-tetrahydrofuran-3-yl)- 2H-indazol-5-yl)-1-(2,6- difluorophenyl)-6-oxo-1,6- dihydropyridazine-3- carboxamide | HCl Salt | LC-MS m/z: 536.2(M + 1)⁺. ¹H NMR (500 MHz, CD₃OD) δ 8.47 (s, 1H), 8.24 (d, J = 9.8 Hz, 1H), 7.79 (d, J = 9.2 Hz, 1H), 7.71-7.65 (m, 1H), 7.36 (d, J = 8.5 Hz, 1H), 7.34-7.28 (m, 3H), 5.39- 5.29 (m, 1H), 4.27-4.22 (m, 1H), 4.20- 4.14 (m, 2H), 3.98 (dt, J = 5.8, 8.5 Hz, 1H), 3.90-3.82 (m, 2H), 3.45-3.41 (m, 1H), 3.09 (dd, J = 5.0, 9.4 Hz, 1H), 2.70-2.57 (m, 1H), 2.50-2.42 (m, 1H), 2.38-2.28 (m, 1H), 1.10 (d, J = 6.9 Hz, 3H). |
| Example 328 | 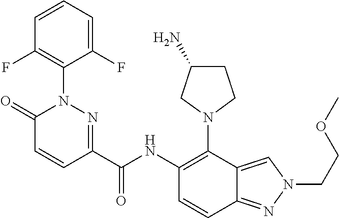 [SMILES: COCCn1cc2c(N3CC[C@@H](N)C3)c(NC(=O)c3ccc(=O)n(-c4c(F)cccc4F)n3)ccc2n1] (R)-N-(4-(3- aminopyrrolidin-1-yl)-2- (2-methoxyethyl)-2H- indazol-5-yl)-1-(2,6- difluorophenyl)-6-oxo-1,6- dihydropyridazine-3- carboxamide | HCOOH salt | LC-MS m/z: 510.1 (M + 1)⁺. ¹H NMR (400 MHz, CD₃OD) δ 8.72 (s, 1H), 8.20 (d, J = 9.9 Hz, 1H), 7.71-7.62 (m, 2H), 7.34-7.21 (m, 3H), 7.34-7.21 (m, 1H), 4.65 (t, J = 5.0 Hz, 2H), 3.93-3.84 (m, 4H), 3.82-3.74 (m, 1H), 3.57-3.49 (m, 2H), 3.33 (s, 3H), 2.36 (qd, J = 7.1, 14.0 Hz, 1H), 2.11-1.97 (m, 1H). |
| Example 329 | 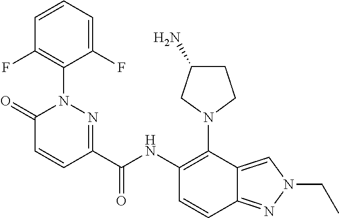 [SMILES: CCn1cc2c(N3CC[C@@H](N)C3)c(NC(=O)c3ccc(=O)n(-c4c(F)cccc4F)n3)ccc2n1] (R)-N-(4-(3- aminopyrrolidin-1-yl)-2- ethyl-2H-indazol-5-yl)-1- (2,6-difluorophenyl)-6- oxo-1,6- dihydropyridazine-3- carboxamide | HCOOH salt | LC-MS m/z: 480.1M + 1)⁺. ¹H NMR (400 MHz, CD₃OD) δ 8.73 (s, 1H), 8.22 (d, J = 9.8 Hz, 1H), 7.77-7.63 (m, 2H), 7.39-7.15 (m, 4H), 4.56 (q, J = 7.4 Hz, 2H), 3.89 (br d, J = 6.7 Hz, 2H), 3.83-3.74 (m, 1H), 3.62-3.48 (m, 2H), 2.38 (br dd, J = 7.0, 13.5 Hz, 1H), 2.14- 2.01 (m, 1H), 1.65 (t, J = 7.3 Hz, 3H). |
| Example 330 | 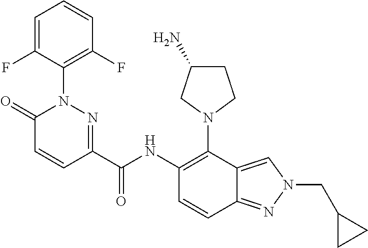 [SMILES: N[C@@H]1CCN(c2c(NC(=O)c3ccc(=O)n(-c4c(F)cccc4F)n3)ccc3nn(CC4CC4)cc23)C1] (R)-N-(4-(3- aminopyrrolidin-1-yl)-2- (cyclopropylmethyl)-2H- indazol-5-yl)-1-(2,6- difluorophenyl)-6-oxo-1,6- dihydropyridazine-3- carboxamide | HCOOH salt | LC-MS m/z: 506.2M + 1)⁺. ¹H NMR (500 MHz, CD₃OD) δ 8.81-8.71 (m, 1H), 8.29-8.20 (m, 1H), 7.76-7.66 (m, 2H), 7.44-7.27 (m, 4H), 4.42-4.30 (m, 2H), 3.93-3.87 (m, 2H), 3.84-3.78 (m, 1H), 3.60-3.51 (m, 2H), 2.44-2.31 (m, 1H), 2.13-1.99 (m, 1H), 1.53-1.42 (m, 1H), 0.75-0.66 (m, 2H), 0.57-0.52 (m, 2H). |
| Example 331 | 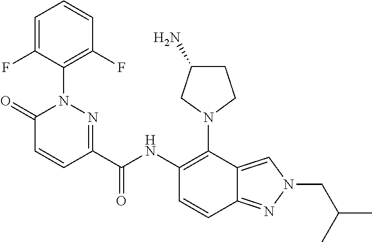 [SMILES: CC(C)Cn1cc2c(N3CC[C@@H](N)C3)c(NC(=O)c3ccc(=O)n(-c4c(F)cccc4F)n3)ccc2n1] (R)- N-(4-(3-aminopyrrolidin- 1-yl)-2-isobutyl-2H- indazol-5-yl)-1-(2,6- difluorophenyl)-6-oxo-1,6- dihydropyridazine-3- carboxamide | HCOOH salt | LC-MS m/z: 508.1M + 1)⁺. ¹H NMR (400 MHz, CD₃OD) δ 8.98 (s, 1H), 8.22 (d, J = 9.8 Hz, 1H), 7.71-7.63 (m, 2H), 7.36-7.28 (m, 3H), 7.24 (d, J = 8.9 Hz, 1H), 4.39 (d, J = 7.5 Hz, 2H), 4.01- 3.87 (m, 3H), 3.72-3.57 (m, 2H), 2.40 (td, J = 6.9, 13.8 Hz, 2H), 2.17-2.10 (m, 1H), 1.01 (d, J = 6.7 Hz, 6H). |
| Example 332 | 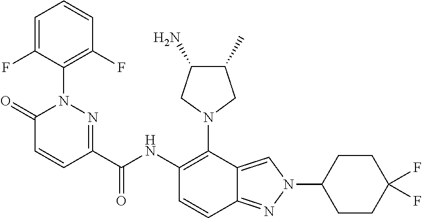 [SMILES: C[C@@H]1CN(c2c(NC(=O)c3ccc(=O)n(-c4c(F)cccc4F)n3)ccc3nn(C4CCC(F)(F)CC4)cc23)C[C@@H]1N] N-(4-((3R,4R)-3-amino-4- methylpyrrolidin-1-yl)-2- (4,4-difluorocyclohexyl)- 2H-indazol-5-yl)-1-(2,6- difluorophenyl)-6-oxo-1,6- dihydropyridazine-3- carboxamide | HCOOH salt | LC-MS m/z: 584.3(M + 1)⁺. ¹H NMR (400 MHz, CD₃OD) δ 8.49 (d, J = 1.0 Hz, 1H), 8.21 (d, J = 9.8 Hz, 1H), 7.71 (d, J = 9.1 Hz, 1H), 7.69-7.61 (m, 1H), 7.30 (dq, J = 8.6, 3.1 Hz, 3H), 7.20 (dd, J = 9.1, 0.9 Hz, 1H), 4.64 (d, J = 15.9 Hz, 2H), 3.76 (dd, J = 9.5, 5.9 Hz, 1H), 3.46- 3.39 (m, 2H), 3.28-3.18 (m, 2H), 2.40- 2.18 (m, 6H), 2.17-1.98 (m, 2H), 0.99 (d, J = 7.0 Hz, 3H). |
| Example 333 | 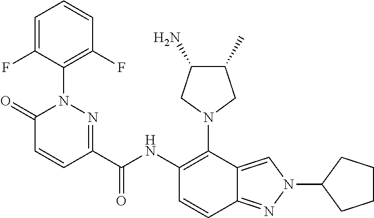 [SMILES: C[C@@H]1CN(c2c(NC(=O)c3ccc(=O)n(-c4c(F)cccc4F)n3)ccc3nn(C4CCCC4)cc23)C[C@@H]1N] N-(4-((3R,4R)-3-amino-4- methylpyrrolidin-1-yl)-2- cyclopentyl-2H-indazol-5- yl)-1-(2,6-difluorophenyl)- 6-oxo-1,6- dihydropyridazine-3- carboxamide | HCOOH salt | LC-MS m/z: 534.2 (M + 1)⁺. ¹H NMR (400 MHz, CD₃OD) δ 8.44 (d, J = 1.0 Hz, 1H), 8.20 (d, J = 9.8 Hz, 1H), 7.66 (tt, J = 8.6, 6.2 Hz, 1H), 7.60 (d, J = 9.1 Hz, 1H), 7.35-7.20 (m, 4H), 4.97 (p, J = 7.4 Hz, 1H), 3.90 (dd, J = 10.2, 6.0 Hz, 1H), 3.66 (q, J = 5.9 Hz, 1H), 3.54 (dd, J = 9.2, 7.4 Hz, 1H), 3.40-3.32 (m, 2H), 2.48 (p, J = 7.1 Hz, 1H), 2.28 (p, J = 6.3 Hz, 2H), 2.15 (dq, J = 12.8, 7.0 Hz, 2H), 2.03-1.92 (m, 2H), 1.87-1.74 (m, 2H), 1.08 (d, J = 7.1 Hz, 3H). |
| Example 334 | 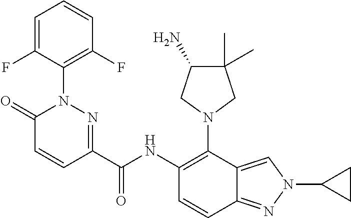 [SMILES: CC1(C)CN(c2c(NC(=O)c3ccc(=O)n(-c4c(F)cccc4F)n3)ccc3nn(C4CC4)cc23)C[C@@H]1N] (R)-N-(4-(4-amino-3,3- dimethylpyrrolidin-1-yl)-2- cyclopropyl-2H-indazol-5- yl)-1-(2,6-difluorophenyl)- 6-oxo-1,6- dihydropyridazine-3- carboxamide | HCOOH salt | LC-MS m/z: 520.2 (M + 1)⁺. ¹H NMR (500 MHz, CD₃OD) δ 8.63 (s, 1H), 8.22 (d, J = 9.8 Hz, 1H), 7.76-7.61 (m, 2H), 7.34-7.17 (m, 4H), 4.12-3.94 (m, 2H), 3.55 (d, J = 9.2 Hz, 1H), 3.48- 3.44 (m, 1H), 3.41 (dd, J = 5.3, 10.5 Hz, 1H), 3.35 (s, 1H), 1.38-1.33 (m, 2H), 1.25- 1.21 (m, 2H), 1.16 (s, 3H), 1.14 (s, 3H). |
| Example 335 | 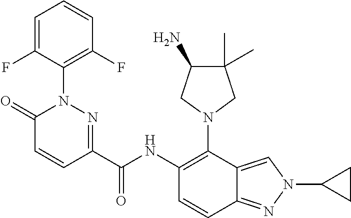 [SMILES: CC1(C)CN(c2c(NC(=O)c3ccc(=O)n(-c4c(F)cccc4F)n3)ccc3nn(C4CC4)cc23)C[C@H]1N] (S)-N-(4-(4-amino-3,3- dimethylpyrrolidin-1-yl)-2- cyclopropyl-2H-indazol-5- yl)-1-(2,6-difluorophenyl)- 6-oxo-1,6- dihydropyridazine-3- carboxamide | HCOOH salt | LC-MS m/z: 520.2 (M + 1)⁺. ¹H NMR (500 MHz, CD₃OD) δ 8.71 (s, 1H), 8.21 (d, J = 9.9 Hz, 1H), 7.73-7.61 (m, 2H), 7.33-7.17 (m, 4H), 4.09-3.96 (m, 2H), 3.59 (d, J = 9.2 Hz, 1H), 3.50- 3.40 (m, 2H), 3.37 (d, J = 9.2 Hz, 1H), 1.40- 1.36 (m, 2H), 1.27-1.22 (m, 2H), 1.17 (s, 3H), 1.14 (s, 3H). |
| Example 336 | 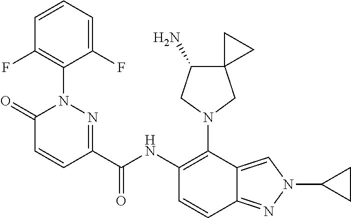 [SMILES: N[C@H]1CN(c2c(NC(=O)c3ccc(=O)n(-c4c(F)cccc4F)n3)ccc3nn(C4CC4)cc23)CC12CC2] (R)-N-(4-(7-amino-5- azaspiro[2.4]heptan-5-yl)- 2-cyclopropyl-2H-indazol- 5-yl)-1-(2,6- difluorophenyl)-6-oxo-1,6- dihydropyridazine-3- carboxamide | HCl Salt | LC-MS m/z: 518.2 (M + 1)⁺. ¹H NMR (400 MHz, CD₃OD) δ 8.53 (s, 1H), 8.23 (d, J = 9.8 Hz, 1H), 7.73-7.63 (m, 1H), 7.59 (d, J = 9.1 Hz, 1H), 7.35- 7.23 (m, 4H), 4.18 (dd, J = 10.7, 5.5 Hz, 1H), 4.03 (dq, J = 7.5, 3.8 Hz, 1H), 3.96 (d, J = 9.2 Hz, 1H), 3.76-3.68 (m, 1H), 3.61 (d, J = 11.0 Hz, 1H), 3.39 (d, J = 4.4 Hz, 1H), 3.09 (d, J = 9.2 Hz, 1H), 1.33 (d, J = 3.1 Hz, 2H), 1.23 (dd, J = 7.5, 5.2 Hz, 2H), 1.02 (dt, J = 10.7, 5.8 Hz, 1H), 0.88-0.80 (m, 1H), 0.79-0.67 (m, 1H). |
| Example 337 | 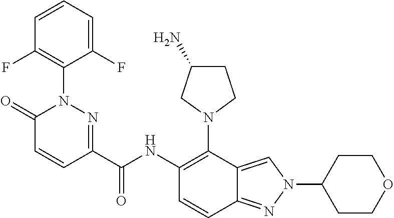 [SMILES: N[C@@H]1CCN(c2c(NC(=O)c3ccc(=O)n(-c4c(F)cccc4F)n3)ccc3nn(C4CCOCC4)cc23)C1] (R)-N-(4-(3- aminopyrrolidin-1-yl)-2- (tetrahydro-2H-pyran-4- yl)-2H-indazol-5-yl)-1-(2,6- difluorophenyl)-6-oxo-1,6- dihydropyridazine-3- carboxamide | HCl Salt | LC-MS m/z: 536.2(M + 1)⁺. ¹H NMR (500 MHz, CD₃OD) δ 9.03 (s, 1H), 8.22 (d, J = 9.8 Hz, 1H), 7.79-7.62 (m, 2H), 7.44-7.23 (m, 4H), 5.00-4.94 (m, 1H), 4.17 (br dd, J = 3.7, 11.7 Hz, 2H), 4.07-3.89 (m, 3H), 3.67 (br t, J = 11.2 Hz, 4H), 2.43 (br d, J = 6.6 Hz, 1H), 2.37-2.27 (m, 2H), 2.26-2.14 (m, 3H). |
| Example 338 | 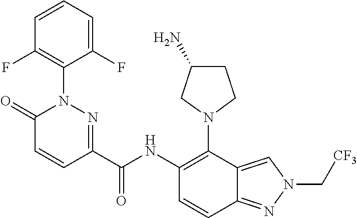 [SMILES: N[C@@H]1CCN(c2c(NC(=O)c3ccc(=O)n(-c4c(F)cccc4F)n3)ccc3nn(CC(F)(F)F)cc23)C1] (R)-N-(4-(3- aminopyrrolidin-1-yl)-2- (2,2,2-trifluoroethyl)-2H- indazol-5-yl)-1-(2,6- difluorophenyl)-6-oxo-1,6- dihydropyridazine-3- carboxamide | HCl Salt | LC-MS m/z: 534.1(M + 1)⁺. ¹H NMR (400 MHz, CD₃OD) δ 8.71-8.63 (m, 1H), 8.26-8.16 (m, 1H), 7.74-7.59 (m, 2H), 7.38-7.20 (m, 4H), 5.30-5.21 (m, 2H), 3.90-3.81 (m, 2H), 3.79-3.70 (m, 1H), 3.55-3.44 (m, 2H), 2.41-2.27 (m, 1H), 2.10-1.97 (m, 1H). |
| Example 339 | 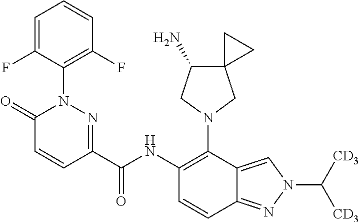 [SMILES: [2H]C([2H])([2H])C(n1cc2c(N3C[C@H](N)C4(CC4)C3)c(NC(=O)c3ccc(=O)n(-c4c(F)cccc4F)n3)ccc2n1)C([2H])([2H])[2H]] (R)-N-(4-(3- aminopyrrolidin-1-yl)-2- ethyl-2H-indazol-5-yl)-1- (2,6-difluorophenyl)-6- oxo-1,6- dihydropyridazine-3- carboxamide | HCl salt | LC-MS m/z: 526.3 (M + 1)⁺ ¹H NMR (400 MHz, CD₃OD) δ 8.64 (s, 1H), 8.21 (d, J = 9.8 Hz, 1H), 7.70-7.63 (m, 1H), 7.60 (d, J = 9.1 Hz, 1H), 7.32-7.23 (m, 4H), 4.93-4.89 (m, 1H), 4.21 (dd, J = 5.5, 10.7 Hz, 1H), 4.03 (d, J = 9.3 Hz, 1H), 3.64 (dd, J = 2.1, 10.8 Hz, 1H), 3.38 (br d, J = 3.7 Hz, 1H), 3.12 (d, J = 9.2 Hz, 1H), 1.04-0.99 (m, 1H), 0.87-0.81 (m, 1H), 0.77-0.70 (m, 2H). |

### Example 340: 1-(2,6-difluorophenyl)-N-{4-[(7R)-7-amino-5-azaspiro[2.4]heptan-5-yl]-1-(prop-2-yl)benzo[d][1,2,3]triazol-5-yl}-6-oxo-1,2-diazine-3-carboxamide

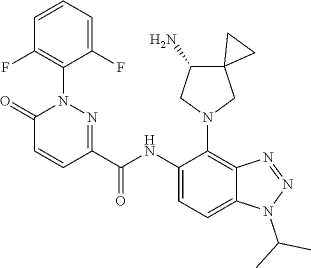 [SMILES: CC(C)n1nnc2c(N3C[C@H](N)C4(CC4)C3)c(NC(=O)c3ccc(=O)n(-c4c(F)cccc4F)n3)ccc21]

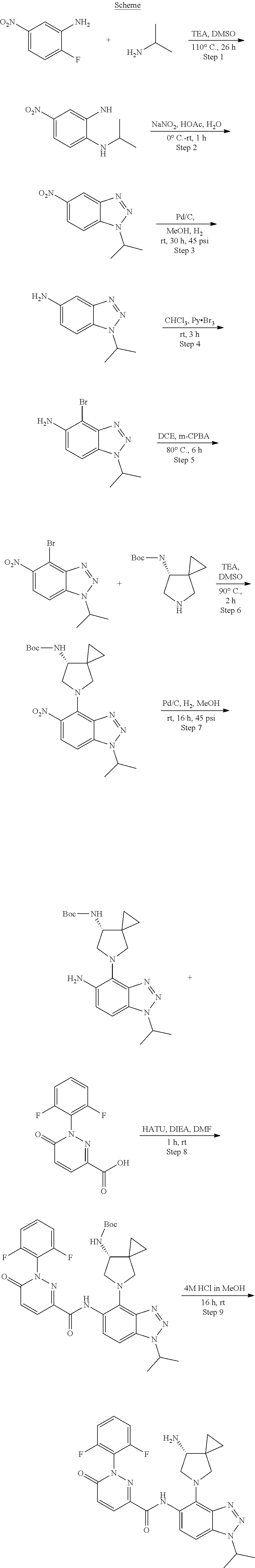

### Step 1: 5-nitro-2-(prop-2-ylamino)aniline

To a solution of 2-fluoro-5-nitroaniline (1 g, 6.405 mmol) in DMSO (10 mL) was added propan-2-amine (0.40 g, 6.726 mmol) and TEA (0.890 mL, 6.405 mmol). The resulting mixture was stirred for 16 hours at 80° C. to give a black brown solution. LCMS showed most of the starting material was remained and one new peak with desired mass was detected. More additional propan-2-amine (0.40 g, 6.726 mmol) was added to the reaction. The mixture was warmed to 110° C. and stirred for another 10 hours to give a black brown solution. LCMS showed most of the starting material was consumed and one major peak with desired mass was detected. No operation and the reaction was used into next step directly.

LC-MS m/z: 196.1 (M+1)⁺

### Step 2: 5-nitro-1-(prop-2-yl)benzo[d][1,2,3]triazole

To a solution of 5-nitro-2-(prop-2-ylamino)aniline (1.25 g, 6.403 mmol) in DMF was added HOAc (4 mL) and sodium nitrite (0.66 g, 9.605 mmol) in H₂O (3 mL) at 0^{˜}5° C. The reaction was stirred for 1 hour at rt to give a brown suspension. LCMS showed the starting material was consumed up and one major peak with desired mass was detected. The reaction was filtered and washed with water, the filter cake was dried by oil pump to afford 5-nitro-1-(prop-2-yl)benzo[d][1,2,3]triazole (1.17 g, Yield: 88.61%) as a brown solid.

LC-MS m/z: 207.1 (M+1)⁺

### Step 3: 1-(prop-2-yl)benzo[d][1,2,3]triazol-5-amine

To a solution of 5-nitro-1-(prop-2-yl)benzo[d][1,2,3]triazole (1.17 g, 5.674 mmol) in MeOH (15 mL) was added Pd/C 10% (0.1 g, 0.325 mmol) under N₂. The resulting mixture was degassed with H₂ several times and stirred for 30 hours at rt under 45 psi to give a black suspension. LCMS showed the starting material was consumed and one major peak with the desired mass was detected. The reaction was filtered through a pad of Celite and the filtrate was concentrated to afford 1-(prop-2-yl)benzo[d][1,2,3]triazol-5-amine (0.98 g, Yield: 98.02%) as a brown oil.

LC-MS m/z: 177.1 (M+1)⁺

### Step 4: 4-bromo-1-ethylbenzo[d][1,2,3]triazol-5-amine

To a solution of 1-(prop-2-yl)benzo[d][1,2,3]triazol-5-amine (0.98 g, 5.561 mmol) in CHCl₃ (20 mL) was added Pyridinium tribromide (1.77 g, 5.561 mmol). The reaction was stirred for 3 hours at rt to give a black brown solution. LCMS showed most of the starting material was consumed up and one major peak with desired mass was detected. The reaction was filtered and the filter cake was washed with hexane. The solid was dissolved in EA and neutralized with con. NaHCO₃. The organic layer was separated and dried over Na₂SO₄, filtered and concentrated to afford 4-bromo-1-ethylbenzo[d][1,2,3]triazol-5-amine (1.2 g, Yield: 89.55%) as black brown oil.

LC-MS m/z: 255.1 (M+1)⁺

### Step 5: 4-bromo-5-nitro-1-(prop-2-yl)benzo[d][1,2,3]triazole

To a solution of 4-bromo-1-(prop-2-yl)benzo[d][1,2,3]triazol-5-amine (1.2 g, 4.704 mmol) in DCE (15 mL) was added m-CPBA (3.82 g, 18.816 mmol). The reaction was stirred for 6 hours at 80° C. to give a brown solution. LCMS showed the starting material was consumed up and one major peak with desired mass was detected. The reaction was quenched with NaHCO₃ aq. and extracted with DCM (30 mL×3). The combined organic layers were washed with brine, dried over Na₂SO₄, filtered and concentrated to afford 4-bromo-5-nitro-1-(prop-2-yl)benzo[d][1,2,3]triazole (1.3 g, Yield: 97.01%) as a brown solid.

LC-MS m/z: 285.1 (M+1)⁺

### Step 6: 2-methylpropan-2-yl {[(7R)-5-[5-nitro-1-(prop-2-yl)benzo[d][1,2,3]triazol-4-yl]-5-azaspiro[2.4]heptan-7-yl]amino}methanoate

To a solution of 4-bromo-5-nitro-1-(prop-2-yl)benzo[d][1,2,3]triazole (200 mg, 0.702 mmol) and 2-methylpropan-2-yl{[(7R)-5-azaspiro[2.4]heptan-7-yl]amino}methanoate chlorane (183.23 mg, 0.737 mmol) in DMSO (3 mL) was added TEA (0.244 mL, 1.754 mmol). The reaction was stirred for 2 hours at 90° C. to give a brown solution. LCMS showed the starting material was consumed up and one major peak with desired mass was detected. The reaction was quenched with H₂O and extracted with EA (10 mL×3). The combined organic layers were washed with brine, dried over Na₂SO₄, filtered and concentrated to afford 2-methylpropan-2-yl{[(7R)-5-[5-nitro-1-(prop-2-yl)benzo[d][1,2,3]triazol-4-yl]-5-azaspiro[2.4]heptan-7-yl]amino}methanoate (290 mg, Yield: 99.26%) as brown oil.

LC-MS m/z: 417.2 (M+1)⁺

### Step 7: 2-methylpropan-2-yl{[(7R)-5-[5-amino-1-(prop-2-yl)benzo[d][1,2,3]triazol-4-yl]-5-azaspiro[2.4]heptan-7-yl]amino}methanoate

To a solution of 2-methylpropan-2-yl{[(7R)-5-[5-nitro-1-(prop-2-yl)benzo[d][1,2,3]triazol-4-yl]-5-azaspiro[2.4]heptan-7-yl]amino}methanoate (290 mg, 0.696 mmol) in MeOH (5 mL) was added Pd/C 10% (50 mg, 0.442 mmol) under N₂. The resulting mixture was degassed with H₂ several times and stirred for 16 hours at rt under 45 psi to give a black suspension. LCMS showed the starting material was consumed and one major peak with the desired mass was detected. The reaction was filtered through a pad of Celite and the filtrate was concentrated to afford 2-methylpropan-2-yl{[(7R)-5-[5-amino-1-(prop-2-yl)benzo[d][1,2,3]triazol-4-yl]-5-azaspiro[2.4]heptan-7-yl]amino}methanoate (265 mg, Yield: 98.47%) as a brown solid.

LC-MS m/z: 386.3 (M+1)⁺

### Step 8: 2-methylpropan-2-yl{[(7R)-5-[5-({[1-(2,6-difluorophenyl)-6-oxo-1,2-diazin-3-yl]carbonyl}amino)-1-(prop-2-yl)benzo[d][1,2,3]triazol-4-yl]-5-azaspiro[2.4]heptan-7-yl]amino}methanoate

To a solution of 1-(2,6-difluorophenyl)-6-oxo-1,2-diazine-3-carboxylic acid (130 mg, 0.516 mmol) and 2-methylpropan-2-yl {[(7R)-5-[5-amino-1-(prop-2-yl)benzo[d][1,2,3]triazol-4-yl]-5-azaspiro[2.4]heptan-7-yl]amino}methanoate (259.02 mg, 0.670 mmol) in DMF (3 mL) was added DIEA (0.256 mL, 1.547 mmol) and HATU (294.02 mg, 0.773 mmol). The residue was stirred for 1 hour at rt to give a green suspension. LCMS showed the starting material was consumed up and one major peak with desired mass was detected. The reaction was purified by flash column (C₁₈, eluted with a gradient of ACN/H₂O containing 0.5% HCOOH, at a rate of 45 mL/min) to afford 2-methylpropan-2-yl {[(7R)-5-[5-({[1-(2,6-difluorophenyl)-6-oxo-1,2-diazin-3-yl]carbonyl}amino)-1-(prop-2-yl)benzo[d][1,2,3]triazol-4-yl]-5-azaspiro[2.4]heptan-7-yl]amino}methanoate (235 mg, Yield: 73.45%) as a brown solid.

LC-MS m/z: 621.3 (M+1)⁺

### Step 9: 1-(2,6-difluorophenyl)-N-{4-[(7R)-7-amino-5-azaspiro[2.4]heptan-5-yl]-1-(prop-2-yl)benzo[d][1,2,3]triazol-5-yl}-6-oxo-1,2-diazine-3-carboxamide

A solution of 2-methylpropan-2-yl {[(7R)-5-[5-({[1-(2,6-difluorophenyl)-6-oxo-1,2-diazin-3-yl]carbonyl}amino)-1-(prop-2-yl)benzo[d][1,2,3]triazol-4-yl]-5-azaspiro[2.4]heptan-7-yl]amino}methanoate (235 mg, 0.379 mmol) in EA (2 mL) was added 4 M HCl in MeOH (5 mL). The resulting mixture was 16 hours at rt to give a brown solution. LCMS showed the starting material was consumed up and one major peak with desired mass was detected. The reaction was concentrated to dryness. The residue was triturated with MeOH and lyophilized to afford Example 340 (175.72 mg, Yield: 83.24%) as a yellow solid.

LC-MS m/z: 521.2 (M+1)⁺

¹H NMR (400 MHz, CD₃OD) δ 8.22 (d, J=9.9 Hz, 1H), 7.94 (d, J=8.9 Hz, 1H), 7.67 (tt, J=8.6, 6.2 Hz, 1H), 7.43 (d, J=8.9 Hz, 1H), 7.36-7.25 (m, 3H), 5.16 (h, J=6.8 Hz, 1H), 4.30 (dd, J=12.0, 5.3 Hz, 1H), 4.16-4.05 (m, 2H), 3.41 (dd, J=5.3, 1.7 Hz, 1H), 3.20 (d, J=9.7 Hz, 1H), 1.70 (dd, J=6.8, 1.8 Hz, 6H), 1.06 (dt, J=9.7, 5.8 Hz, 1H), 0.82 (dt, J=9.2, 5.7 Hz, 1H), 0.76 (ddd, J=10.5, 6.3, 4.3 Hz, 1H), 0.61 (ddd, J=10.1, 6.2, 4.3 Hz, 1H).

| No. | Structure | Salt form | MS and ¹HNMR |
|---|---|---|---|
| Example 341 | 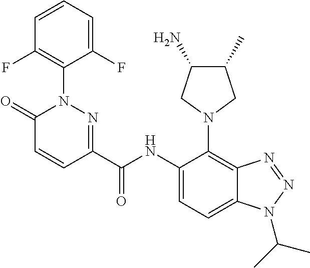 [SMILES: CC(C)n1nnc2c(N3C[C@@H](C)[C@@H](N)C3)c(NC(=O)c3ccc(=O)n(-c4c(F)cccc4F)n3)ccc21] N-(4-((3R,4R)-3-amino-4- methylpyrrolidin-1-yl)-1- isopropyl-1H- benzo[d][1,2,3]triazol-5-yl)-1- (2,6-difluorophenyl)-6-oxo-1,6- dihydropyridazine-3-carboxamide | HCl Salt | LC-MS m/z: 509.2 (M + 1)⁺ ¹H NMR (400 MHz, CD₃OD) δ 8.21 (d, J = 9.9 Hz, 1H), 7.76 (d, J = 8.8 Hz, 1H), 7.66 (tt, J = 8.6, 6.2 Hz, 1H), 7.31 (dt, J = 9.0, 6.6 Hz, 4H), 5.13 (hept, J = 6.7 Hz, 1H), 4.24 (dd, J = 11.8, 5.8 Hz, 1H), 4.02 (dd, J = 11.8, 3.1 Hz, 1H), 3.87 (td, J = 5.9, 3.0 Hz, 1H), 3.78 (dd, J = 10.0, 7.7 Hz, 1H), 3.55 (t, J = 9.4 Hz, 1H), 2.60 (dq, J = 14.7, 7.3 Hz, 1H), 1.69 (dd, J = 6.7, 1.3 Hz, 6H), 1.16 (d, J = 7.1 Hz, 3H). |

### Example 342: N-(4-(4-cyanopyridin-3-yl)-2-(piperazin-1-yl)phenyl)-1-(2,6-difluorophenyl)-6-oxo-1,6-dihydropyridazine-3-carboxamide

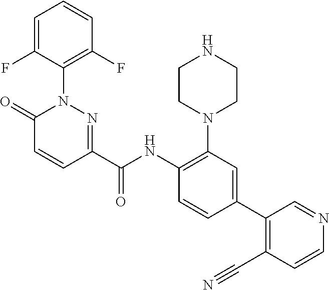 [SMILES: N#Cc1ccncc1-c1ccc(NC(=O)c2ccc(=O)n(-c3c(F)cccc3F)n2)c(N2CCNCC2)c1]

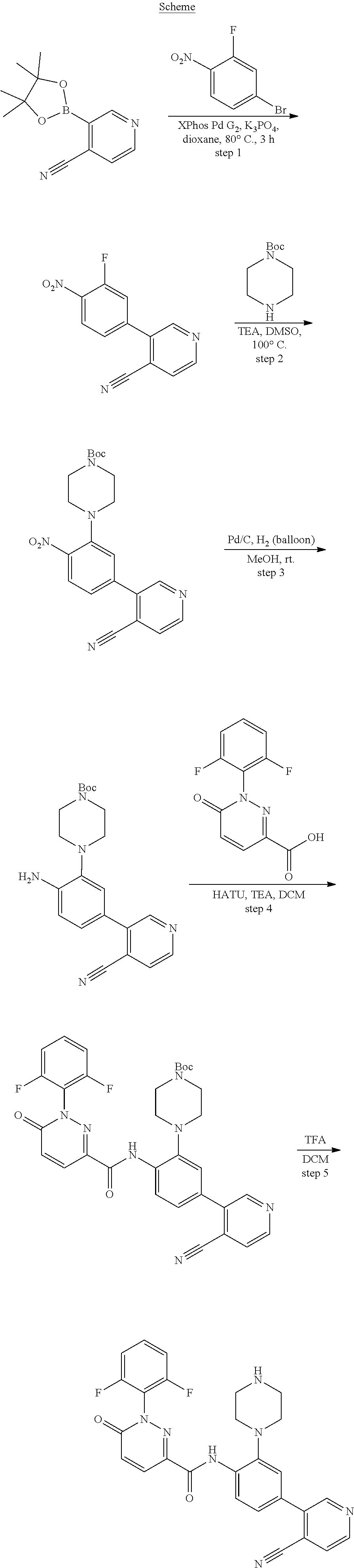

### Step 1: 3-(3-fluoro-4-nitrophenyl)isonicotinonitrile

To a solution of 3-(4,4,5,5-tetramethyl-1,3,2-dioxaborolan-2-yl)pyridine-4-carbonitrile (1 g, 4.35 mmol, 1 eq) in dioxane (20 mL) and H₂O (5 mL) was added 4-bromo-2-fluoro-1-nitro-benzene (1.05 g, 4.78 mmol, 1.1 eq) and K₂CO₃ (1.80 g, 13.04 mmol, 3 eq) and XPhos-Pd-G2 (341.98 mg, 434.65 umol, 0.1 eq). The mixture was stirred at 110° C. for 2 hr. LCMS showed desired mass formed. TLC indicated new spot formed. The reaction mixture was quenched by addition water 25 mL, extracted with EtOAc (30 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0^{˜}50% Ethyl acetate/Petroleum ethergradient). The purified product 3-(3-fluoro-4-nitro-phenyl)pyridine-4-carbonitrile (800 mg, yield: 75.68%) was gave as a light brown solid.

LC-MS m/z: 244.0 (M+1)⁺.

### Step 2: tert-butyl 4-(5-(4-cyanopyridin-3-yl)-2-nitrophenyl)piperazine-1-carboxylate

To a solution of 3-(3-fluoro-4-nitro-phenyl)pyridine-4-carbonitrile (150 mg, 616.79 umol, 1 eq) in DMSO (3 mL) was added tert-butyl piperazine-1-carboxylate (172.32 mg, 925.19 umol, 1.5 eq) and TEA (124.83 mg, 1.23 mmol, 171.70 uL, 2 eq). The mixture was stirred at 100° C. for 2 hr. LCMS show desired mass formed. TLC indicated a new spot produced. The reaction mixture was poured into water (15 mL), extracted with EtOAc (15 mL×3). The combined organic phases were dried over Na₂SO₄, filtered. The filtrate was concentrated. The residue was purified by flash silica gel chromatography (ISCO®; 4 g SepaFlash® Silica Flash Column, Eluent of 0^{˜}60% Ethyl acetate/Petroleum ether gradient). The purified product tert-butyl 4-[5-(4-cyano-3-pyridyl)-2-nitro-phenyl]piperazine-1-carboxylate (240 mg, yield: 95.04%) was gave as a brown oil.

LC-MS m/z: 410.2 (M+1)⁺.

### Step 3: tert-butyl 4-(2-amino-5-(4-cyanopyridin-3-yl)phenyl)piperazine-1-carboxylate

To a solution of tert-butyl 4-[5-(4-cyano-3-pyridyl)-2-nitro-phenyl]piperazine-1-carboxylate (240 mg, 586.17 umol, 1 eq) in MeOH (10 mL) was added Pd/C (62.13 mg, 58.62 umol, 10% purity, 0.1 eq). The mixture was stirred with a balloon of H₂ at 25° C. for 2 hr. LCMS showed the desired mass formed and starting material was consumed. The reaction mixture was filtered. The filtrate was concentrated in vacuum. tert-butyl 4-[2-amino-5-(4-cyano-3-pyridyl)phenyl]piperazine-1-carboxylate (170 mg, yield: 76.43%) was obtained as brown solid.

LC-MS m/z: 380.2 (M+1)⁺.

### Step 4: tert-butyl 4-(5-(4-cyanopyridin-3-yl)-2-(1-(2,6-difluorophenyl)-6-oxo-1,6-dihydropyridazine-3-carboxamido)phenyl)piperazine-1-carboxylate

To a solution of tert-butyl 4-[2-amino-5-(4-cyano-3-pyridyl)phenyl]piperazine-1-carboxylate (150 mg, 395.30 umol, 1 eq) in DMF (3 mL) were added 1-(2,6-difluorophenyl)-6-oxo-pyridazine-3-carboxylic acid (99.69 mg, 395.30 umol, 1 eq) and TEA (80.00 mg, 790.61 umol, 110.04 uL, 2 eq) and HATU (225.46 mg, 592.96 umol, 1.5 eq). The mixture was stirred at 60° C. for 2 hr. LCMS show desired mass formed. TLC indicated new spots produced. The reaction mixture was quenched by addition water (15 mL). The combined organic layers were extracted with EtOAc (5 mL×3), dried over Na₂SO₄, filtered and concentrated under reduced pressure to give a residue. The residue was purified by flash silica gel chromatography (ISCO®; 4 g SepaFlash® Silica Flash Column, Eluent of 0^{˜}30% Ethyl acetate/Petroleum ether gradient). tert-butyl 4-[5-(4-cyano-3-pyridyl)-2-[[1-(2,6-difluorophenyl)-6-oxo-pyridazine-3-carbonyl]amino]phenyl]piperazine-1-carboxylate (200 mg, yield: 82.45%) was gave a light yellow solid.

LC-MS m/z: 614.2 (M+1)⁺.

### Step 5: N-(4-(4-cyanopyridin-3-yl)-2-(piperazin-S-yl)phenyl)-1-(2,6-difluorophenyl)-6-oxo-1,6-dihydropyridazine-3-carboxamide

To a solution of tert-butyl 4-[5-(4-cyano-3-pyridyl)-2-[[1-(2,6-difluorophenyl)-6-oxo-pyridazine-3-carbonyl]amino]phenyl]piperazine-1-carboxylate (200 mg, 325.94 umol, 1 eq) in DCM (2 ml) was added TFA (3.08 g, 27.01 mmol, 2 mL, 82.87 eq). The mixture was stirred at 25° C. for 2 hr. LCMS show desired mass formed. The combined organic phases were concentrated in vacuum to give the crude product. The crude product was purified by pre-HPLC (Boston Prime C18 150λ30 mm×5 am, water (FA)-ACN, Begin B 17, End B 47, Gradient Time (min) 12, 100% B Hold Time (min) 2, Flow Rate (ml/min) 25). Example 342 (60.27 mg, yield: 36.01%) was obtained as a white solid.

¹H NMR (500 MHz, CD₃OD) δ 8.90 (s, 1H), 8.76 (d, J=5.04 Hz, 1H), 8.64 (d, J=8.54 Hz, 1H), 8.27 (d, J=9.77 Hz, 1H), 7.85 (d, J=4.73 Hz, 1H), 7.69-7.76 (m, 1H), 7.66 (d, J=1.98 Hz, 1H), 7.56 (dd, J=8.47, 82.06 Hz, 1H), 7.33-7.40 (m, 3H), 3.09-3.14 (m, 4H), 3.03 (brs, 4H).

| No. | Structure | Salt form | MS and ¹HNMR |
|---|---|---|---|
| Example 343 | 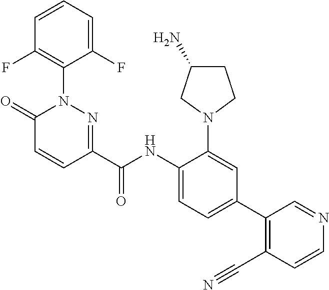 [SMILES: N#Cc1ccncc1-c1ccc(NC(=O)c2ccc(=O)n(-c3c(F)cccc3F)n2)c(N2CC[C@@H](N)C2)c1] (R)-N- (2-(3-aminopyrrolidin-1-yl)-4-(4- cyanopyridin-3-yl)phenyl)-1-(2,6- difluorophenyl)-6-oxo-1,6- dihydropyridazine-3-carboxamide | HCl Salt | LC-MS m/z: 514.2 (M + 1)⁺ ¹H NMR (400 MHz, CD₃OD) δ 9.09 (s, 1H), 8.90 (d, J = 5.5 Hz, 1H), 8.27-8.26 (m, 1H), 8.16 (d, J = 5.5 Hz, 1H), 7.74-7.67 (m, 1H), 7.57 (d, J = 2.0 Hz, 1H), 7.45 (dd, J = 8.4, 2.0 Hz, 1H), 7.39-7.29 (m, 3H), 3.88-3.77 (m, 1H), 3.69- 3.58 (m, 1H), 3.47 (m, 1H), 3.20- 3.25 (m, 2H), 2.35-2.26 (m, 1H), 2.01-1.87 (m, 1H). |
| Example 344 | 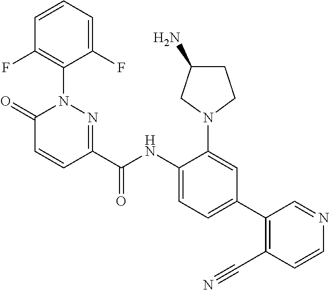 [SMILES: N#Cc1ccncc1-c1ccc(NC(=O)c2ccc(=O)n(-c3c(F)cccc3F)n2)c(N2CC[C@H](N)C2)c1] (S)-N- (2-(3-aminopyrrolidin-1-yl)-4-(4- cyanopyridin-3-yl)phenyl)-1-(2,6- difluorophenyl)-6-oxo-1,6- dihydropyridazine-3-carboxamide | HCl Salt | LC-MS m/z: 514.2 (M + 1)⁺ ¹H NMR (400 MHz, CD₃OD) δ 8.92 (s, 1H), 8.79 (d, 1H), 8.25-8.20 (m, 2H), 7.70 (d,1H), 7.48 (m, 1H), 7.41 (s, 1H), 7.30 (m, 1H), 7.36- 7.32 (m, 1H), 3.67 (s, 1H), 3.65- 3.61 (m, 1H), 3.50-3.46 (m, 1H), 3.25-3.20 (m, 2H), 2.25-2.23 (m, 1H), 1.91-1.88 (m, 1H). |
| Example 345 | 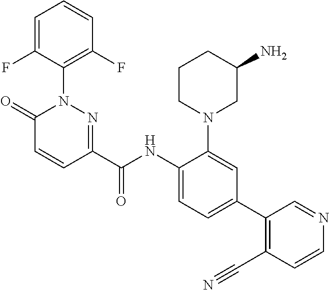 [SMILES: N#Cc1ccncc1-c1ccc(NC(=O)c2ccc(=O)n(-c3c(F)cccc3F)n2)c(N2CCC[C@@H](N)C2)c1] (R)-N- (2-(3-aminopiperidin-1-yl)-4-(4- cyanopyridin-3-yl)phenyl)-1-(2,6- difluorophenyl)-6-oxo-1,6- dihydropyridazine-3-carboxamide | HCl Salt | LC-MS m/z: 528.2 (M + 1)⁺ ¹H NMR (400 MHz, CD₃OD) δ 9.04 (d, 1H), 8.86 (d, 1H), 8.66 (d, 1H), 8.07 (d, 1H), 7.70 (m, 2H), 7.58 (d, 1H), 7.39-7.35 (m, 3H), 3.30 (m, 1H), 3.01-2.74 (m, 3H), 2.71 (m, 3H), 1.86 (m, 1H), 1.74 (m, 1H), 1.51-1.30 (m, 1H). |
| Example 346 | 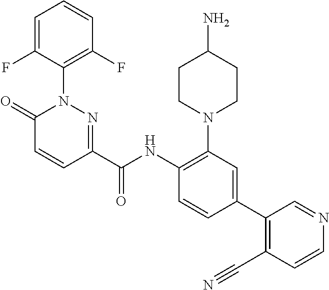 [SMILES: N#Cc1ccncc1-c1ccc(NC(=O)c2ccc(=O)n(-c3c(F)cccc3F)n2)c(N2CCC(N)CC2)c1] N-(2-(4- aminopiperidin-1-yl)-4-(4- cyanopyridin-3-yl)phenyl)-1-(2,6- difluorophenyl)-6-oxo-1,6- dihydropyridazine-3-carboxamide | HCl Salt | LC-MS m/z: 528.2 (M + 1)⁺ ¹H NMR (400 MHz, CD₃OD) δ 8.89 (s, 1H), 8.76 (d, J = 5.0 Hz, 1H), 8.59 (d, J = 8.3 Hz, 1H), 8.28 (d, J = 9.9 Hz, 1H), 7.85 (d, J = 5.1 Hz, 1H), 7.75-7.67 (m, 1H), 7.61 (s, 1H), 7.51 (d, J = 8.5 Hz, 1H), 7.39- 7.30 (m, 3H), 3.22 (d, J = 4.5 Hz, 3H), 2.93 (t, J = 9.6 Hz, 2H), 1.93 (s, 2H), 1.65 (d, J = 9.1 Hz, 2H). |
| Example 347 | 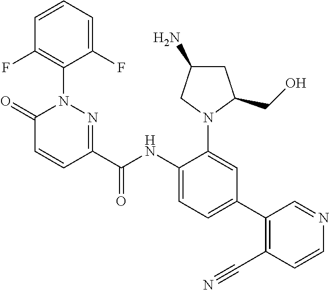 [SMILES: N#Cc1ccncc1-c1ccc(NC(=O)c2ccc(=O)n(-c3c(F)cccc3F)n2)c(N2C[C@@H](N)C[C@H]2CO)c1] N-(2- ((2S,4S)-4-amino-2- (hydroxymethyl)pyrrolidin-1-yl)-4- (4-cyanopyridin-3-yl)phenyl)-1- (2,6-difluorophenyl)-6-oxo-1,6- dihydropyridazine-3-carboxamide | HCl Salt | LC-MS m/z: 544.2 (M + 1)⁺ ¹H NMR (400 MHz, CD₃OD) δ 8.87 (s, 1H), 8.70 (d, 2H), 8.55 (d, 1H), 8.16 (d, 2H) 7.84 (d, 1H), 7.48 (m, 2H), 7.20 (m, 2H), 3.77 (s, 1H), 3.65 (d, 1H), 3.32 (m, 2H), 3.20 (m, 2H), 2.61-2.57 (m, 1H), 1.89- 1.85 (m, 1H). |
| Example 348 | 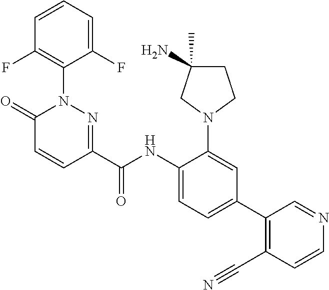 [SMILES: C[C@]1(N)CCN(c2cc(-c3cnccc3C#N)ccc2NC(=O)c2ccc(=O)n(-c3c(F)cccc3F)n2)C1] (S)-N- (2-(3-amino-3-methylpyrrolidin-1- yl)-4-(4-cyanopyridin-3-yl)phenyl)- 1-(2,6-difluorophenyl)-6-oxo-1,6- dihydropyridazine-3-carboxamide | HCl Salt | LC-MS m/z: 528.2 (M + 1)⁺ ¹H NMR (400 MHz, CD₃OD) δ 8.92 (s, 1H), 8.78 (d, 1H), 8.31-8.26 (m, 1H), 7.51 (d, 1H), 7.44-7.42 (m, 1H), 7.41 (s, 1H), 7.36 (d, 1H), 7.32-7.30 (m, 1H), 3.3-3.2 (m, 3H), 2.10-2.07 (m, 1H),1.96-1.90 (m, 1H), 1.34 (s, 3H). |
| Example 349 | 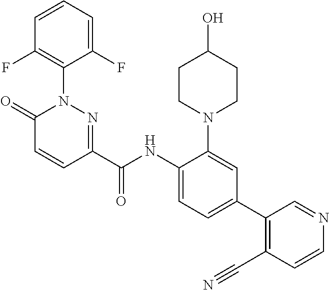 [SMILES: N#Cc1ccncc1-c1ccc(NC(=O)c2ccc(=O)n(-c3c(F)cccc3F)n2)c(N2CCC(O)CC2)c1] N-(4-(4- cyanopyridin-3-yl)-2-(4- hydroxypiperidin-1-yl)phenyl)-1- (2,6-difluorophenyl)-6-oxo-1,6- dihydropyridazine-3-carboxamide | HCl base | LC-MS m/z: 529.2 (M + 1)⁺ ¹H NMR (500 MHz, CD₃OD) δ 9.21 (s, 1H), 8.99 (br d, J = 5.6 Hz, 1H), 8.51-8.33 (m, 3H), 8.27 (d, J = 9.9 Hz, 1H), 7.75-7.64 (m, 2H), 7.40- 7.26 (m, 3H), 3.79 (br s, 1H), 3.40-3.34 (m, 2H), 3.17-2.95 (m, 2H), 1.86 (br d, J = 2.7 Hz, 2H), 1.72-1.57 (m, 2H). |
| Example 350 | 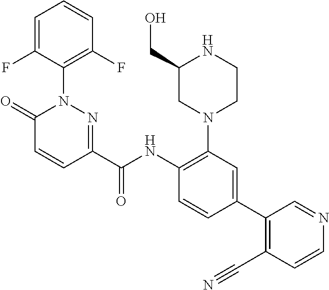 [SMILES: N#Cc1ccncc1-c1ccc(NC(=O)c2ccc(=O)n(-c3c(F)cccc3F)n2)c(N2CCN[C@H](CO)C2)c1] (S)-N- (4-(4-cyanopyridin-3-yl)-2-(3- (hydroxymethyl)piperazin-1- yl)phenyl)-1-(2,6-difluorophenyl)- 6-oxo-1,6-dihydropyridazine-3- carboxamide | HCl base | LC-MS m/z: 544.2 (M + 1)⁺ ¹H NMR (500 MHz, CD₃OD) δ 8.97- 8.92 (m, 1H), 8.82-8.77 (m, 1H), 8.66-8.60 (m, 1H), 8.28-8.22 (m, 1H), 7.96-7.90 (m, 1H), 7.75- 7.66 (m, 2H), 7.61-7.56 (m, 1H), 7.41-7.31 (m, 3H), 3.77-3.67 (m, 1H), 3.65-3.55 (m, 1H), 3.48- 3.37 (m, 1H), 3.31 (td, J = 1.6, 3.2 Hz, 1H), 3.29-3.09 (m, 5H), 3.07- 3.01 (m, 1H), 2.97-2.88 (m, 1H). |
| Example 351 | 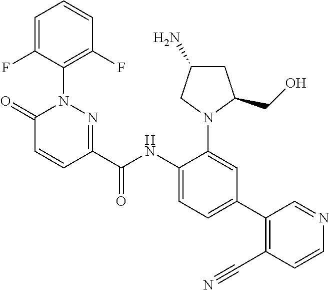 [SMILES: N#Cc1ccncc1-c1ccc(NC(=O)c2ccc(=O)n(-c3c(F)cccc3F)n2)c(N2C[C@H](N)C[C@H]2CO)c1] N-(2- ((2S,4R)-4-amino-2- (hydroxymethyl)pyrrolidin-1-yl)-4- (4-cyanopyridin-3-yl)phenyl)-1- (2,6-difluorophenyl)-6-oxo-1,6- dihydropyridazine-3-carboxamide | HCl base | LC-MS m/z: 544.2 (M + 1)⁺ ¹H NMR (400 MHz, CD₃OD) δ 9.00 (s, 1H), 8.83 (d, J = 5.4 Hz, 1H), 8.42 (d, J = 8.5 Hz, 1H), 8.25 (d, J = 9.8 Hz, 1H), 8.00 (d, J = 5.2 Hz, 1H), 7.75-7.68 (m, 2H), 7.52 (dd, J = 2.0, 8.4 Hz, 1H), 7.38-7.32 (m, 3H), 3.89 (dt, J = 3.4, 7.6 Hz, 1H), 3.80 (dd, J = 6.6, 10.0 Hz, 1H), 3.61 (br dd, J = 4.1, 8.0 Hz, 1H), 3.43-3.41 (m, 2H), 3.02 (dd, J = 5.8, 10.2 Hz, 1H), 2.29-2.18 (m, 1H), 2.14-2.04 (m, 1H). |
| Example 352 | 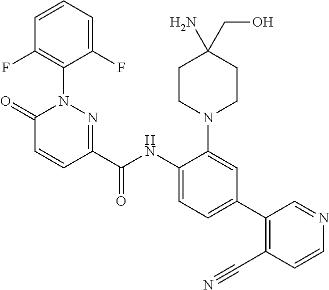 [SMILES: N#Cc1ccncc1-c1ccc(NC(=O)c2ccc(=O)n(-c3c(F)cccc3F)n2)c(N2CCC(N)(CO)CC2)c1] N-(2-(4- amino-4- (hydroxymethyl)piperidin-1-yl)-4- (4-cyanopyridin-3-yl)phenyl)-1- (2,6-difluorophenyl)-6-oxo-1,6- dihydropyridazine-3-carboxamide | HCl base | LC-MS m/z: 558.2 (M + 1)⁺ ¹H NMR (400 MHz, CD₃OD) δ 8.94 (s, 1H), 8.80 (br d, J = 4.9 Hz, 1H), 8.68 (d, J = 8.5 Hz, 1H), 8.30 (d, J = 9.9 Hz, 1H), 7.92 (d, J = 5.1 Hz, 1H), 7.80-7.70 (m, 1H), 7.68 (d, J = 2.0 Hz, 1H), 7.55 (dd, J = 2.0, 8.5 Hz, 1H), 7.42-7.32 (m, 3H), 3.19 (s, 2H), 3.07 (br d, J = 6.1 Hz, 4H), 1.91 (br d, J = 13.1 Hz, 2H), 1.64- 1.51 (m, 2H). |
| Example 353 (isomer 1) | 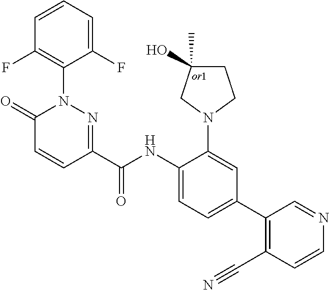 [SMILES: C[C@]1(O)CCN(c2cc(-c3cnccc3C#N)ccc2NC(=O)c2ccc(=O)n(-c3c(F)cccc3F)n2)C1] rel-(R)- N-(4-(4-cyanopyridin-3-yl)-2-(3- hydroxy-3-methylpyrrolidin-1- yl)phenyl)-1-(2,6-difluorophenyl)- 6-oxo-1,6-dihydropyridazine-3- carboxamide | HCl base | LC-MS m/z: 529.2 (M + 1)⁺ ¹H NMR (500 MHz, CD₃OD) δ 8.85 (s, 1H), 8.70 (d, J = 5.2 Hz, 1H), 8.13 (d, J = 9.9 Hz, 1H), 7.92 (br d, J = 7.6 Hz, 1H), 7.83 (d, J = 5.2 Hz, 1H), 7.72-7.52 (m, 2H), 7.38 (br d, J = 7.9 Hz, 1H), 7.25-7.15 (m, 3H), 3.59-3.49 (m, 1H), 3.48- 3.32 (m, 2H), 3.25 (br s, 1H), 1.96- 1.77 (m, 2H), 1.22 (s, 3H). |
| Example 354 (isomer 2) | 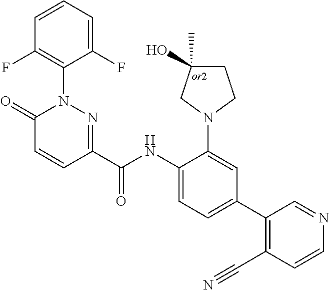 [SMILES: C[C@]1(O)CCN(c2cc(-c3cnccc3C#N)ccc2NC(=O)c2ccc(=O)n(-c3c(F)cccc3F)n2)C1] rel-(R)- N-(4-(4-cyanopyridin-3-yl)-2-(3- hydroxy-3-methylpyrrolidin-1- yl)phenyl)-1-(2,6-difluorophenyl)- 6-oxo-1,6-dihydropyridazine-3- carboxamide | HCl base | LC-MS m/z: 529.2 (M + 1)⁺ ¹H NMR (500 MHz, CD₃OD) δ 8.84 (s, 1H), 8.69 (d, J = 5.0 Hz, 1H), 8.13 (d, J = 9.8 Hz, 1H), 7.94 (br d, J = 7.6 Hz, 1H), 7.81 (d, J = 5.2 Hz, 1H), 7.68-7.52 (m, 2H), 7.35 (br d, J = 7.8 Hz, 1H), 7.25-7.13 (m, 3H), 3.50 (br d, J = 7.3 Hz, 1H), 3.43-3.28 (m, 2H), 3.22 (br s, 1H), 1.95-1.74 (m, 2H), 1.23- 1.19 (m, 3H). |
| Example 355 (isomer 1) | 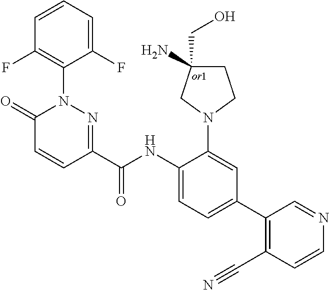 [SMILES: N#Cc1ccncc1-c1ccc(NC(=O)c2ccc(=O)n(-c3c(F)cccc3F)n2)c(N2CC[C@@](N)(CO)C2)c1] rel-(R)- N-(2-(3-amino-3- (hydroxymethyl)pyrrolidin-1-yl)-4- (4-cyanopyridin-3-yl)phenyl)-1- (2,6-difluorophenyl)-6-oxo-1,6- dihydropyridazine-3-carboxamide | HCl base | LC-MS m/z: 544.2 (M + 1)⁺ ¹H NMR (500 MHz, CD₃OD) δ 8.87 (s, 1H), 8.75 (d, J = 5.0 Hz, 1H), 8.40 (d, J = 8.4 Hz, 1H), 8.26 (d, J = 9.8 Hz, 1H), 7.83 (d, J = 5.0 Hz, 1H), 7.76-7.65 (m, 1H), 7.53 (d, J = 1.8 Hz, 1H), 7.45 (dd, J = 2.0, 8.4 Hz, 1H), 7.38-7.29 (m, 3H), 3.46 (dd, J = 6.6, 10.7 Hz, 2H), 3.27- 3.09 (m, 4H), 1.97-1.88 (m, 1H), 1.79-1.67 (m, 1H). |
| Example 356 (isomer 2) | 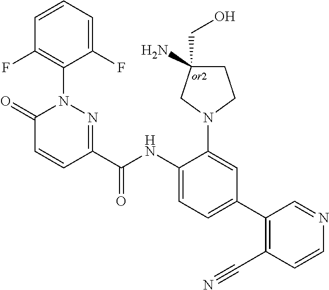 [SMILES: N#Cc1ccncc1-c1ccc(NC(=O)c2ccc(=O)n(-c3c(F)cccc3F)n2)c(N2CC[C@@](N)(CO)C2)c1] rel-(R)- N-(2-(3-amino-3- (hydroxymethyl)pyrrolidin-1-yl)-4- (4-cyanopyridin-3-yl)phenyl)-1- (2,6-difluorophenyl)-6-oxo-1,6- dihydropyridazine-3-carboxamide | HCl base | LC-MS m/z: 544.2 (M + 1)⁺ ¹H NMR (500 MHz, CD₃OD) δ 8.87 (s, 1H), 8.75 (d, J = 5.0 Hz, 1H), 8.40 (d, J = 8.4 Hz, 1H), 8.26 (d, J = 9.9 Hz, 1H), 7.83 (d, J = 5.0 Hz, 1H), 7.74-7.64 (m, 1H), 7.53 (d, J = 2.0 Hz, 1H), 7.45 (dd, J = 2.0, 8.4 Hz, 1H), 7.40-7.28 (m, 3H), 3.46 (dd, J = 7.6, 10.8 Hz, 2H), 3.29- 3.09 (m, 4H), 2.00-1.89 (m, 1H), 1.73 (td, J = 8.2, 13.8 Hz, 1H). |
| Example 357 | 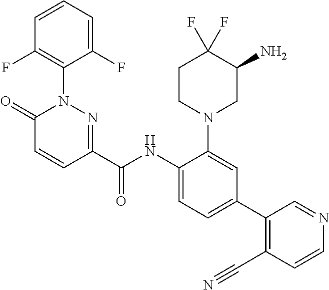 [SMILES: N#Cc1ccncc1-c1ccc(NC(=O)c2ccc(=O)n(-c3c(F)cccc3F)n2)c(N2CCC(F)(F)[C@@H](N)C2)c1] (S)-N- (2-(3-amino-4,4-difluoropiperidin- 1-yl)-4-(4-cyanopyridin-3- yl)phenyl)-1-(2,6-difluorophenyl)- 6-oxo-1,6-dihydropyridazine-3- carboxamide | HCl base | LC-MS m/z: 564.2 (M + 1)⁺ ¹H NMR (500 MHz, CD₃OD) δ 8.88 (m, 1H), 8.76-8.74 (m, 1H), 8.65- 8.63 (m, 1H), 8.29-8.26 (m, 1H), 7.84-7.83 (m, 1H), 7.66 (m, 2H), 7.57-7.55 (m, 1H), 7.36-7.32 (m, 3H), 3.40-3.37 (m, 2H), 3.15- 3.11 (m, 2H), 3.05-3.00 (m, 1H), 2.24-2.22 (m, 1H), 1.90-1.83 (m, 1H). |
| Example 358 (isomer 1) | 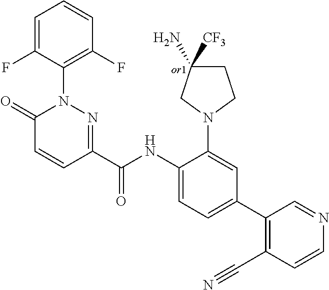 [SMILES: N#Cc1ccncc1-c1ccc(NC(=O)c2ccc(=O)n(-c3c(F)cccc3F)n2)c(N2CC[C@](N)(C(F)(F)F)C2)c1] (R)-N- (2-(3-amino-3- (trifluoromethyl)pyrrolidin-1-yl)-4- (4-cyanopyridin-3-yl)phenyl)-1- (2,6-difluorophenyl)-6-oxo-1,6- dihydropyridazine-3-carboxamide | HCl base | LC-MS m/z: 582.2 (M + 1)⁺ ¹H NMR (400 MHz, CD₃OD) δ 8.93- 8.88 (m, 1H), 8.79-8.75 (m, 1H), 8.51-8.45 (m, 1H), 8.29-8.24 (m, 1H), 7.89-7.83 (m, 1H), 7.72- 7.64 (m, 1H), 7.63-7.58 (m, 1H), 7.56-7.49 (m, 1H), 7.35-7.25 (m, 3H), 3.78 (br d, J = 8.1 Hz, 1H), 3.50-3.36 (m, 3H), 2.38-2.28 (m, 1H), 2.21-2.11 (m, 1H). |
| Example 359 (isomer 2) | 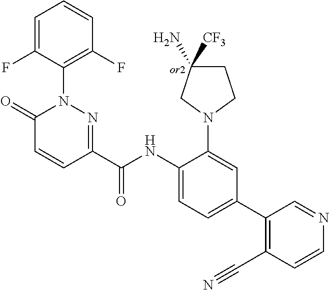 [SMILES: N#Cc1ccncc1-c1ccc(NC(=O)c2ccc(=O)n(-c3c(F)cccc3F)n2)c(N2CC[C@](N)(C(F)(F)F)C2)c1] (R)-N- (2-(3-amino-3- (trifluoromethyl)pyrrolidin-1-yl)-4- (4-cyanopyridin-3-yl)phenyl)-1- (2,6-difluorophenyl)-6-oxo-1,6- dihydropyridazine-3-carboxamide | HCl base | LC-MS m/z: 582.2 (M + 1)⁺ ¹H NMR (400 MHz, CD₃OD) δ 8.96- 8.92 (m, 1H), 8.82-8.76 (m, 1H), 8.54-8.46 (m, 1H), 8.31-8.24 (m, 1H), 7.95-7.89 (m, 1H), 7.72- 7.65 (m, 1H), 7.65-7.63 (m, 1H), 7.57-7.51 (m, 1H), 7.35-7.25 (m, 3H), 3.86-3.80 (m, 1H), 3.50- 3.35 (m, 3H), 2.38-2.28 (m, 1H), 2.23-2.13 (m, 1H). |
| Example 360 | 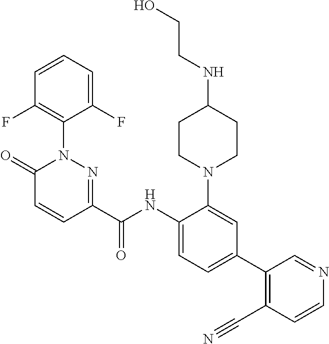 [SMILES: N#Cc1ccncc1-c1ccc(NC(=O)c2ccc(=O)n(-c3c(F)cccc3F)n2)c(N2CCC(NCCO)CC2)c1] N-(4-(4- cyanopyridin-3-yl)-2-(4-((2- hydroxyethyl)amino)piperidin-1- yl)phenyl)-1-(2,6-difluorophenyl)- 6-oxo-1,6-dihydropyridazine-3- carboxamide | HCl base | LC-MS m/z: 572.2 (M + 1)+ 1H NMR (400 MHz, CD₃OD) δ 8.87 (s, 1H), 8.74 (d, J = 5.1 Hz, 1H), 8.56 (d, J = 8.5 Hz, 1H), 8.26 (d, J = 9.8 Hz, 1H), 7.83 (d, J = 5.1 Hz, 1H), 7.69 (t, J = 7.4 Hz, 1H), 7.59 (s, 1H), 7.48 (dd, J = 8.5, 2.0 Hz, 1H), 7.34 (t, J = 9.4 Hz, 3H), 3.83 (t, J = 5.2 Hz, 2H), 3.24 (s, 3H), 2.99 (d, J = 5.2 Hz, 2H), 2.91 (t, J = 11.1 Hz, 2H), 2.02 (d, J = 12.4 Hz, 2H), 1.65 (d, J = 11.8 Hz, 2H). |
| Example 361 | 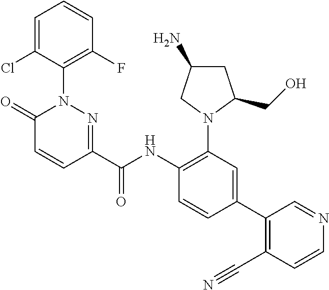 [SMILES: N#Cc1ccncc1-c1ccc(NC(=O)c2ccc(=O)n(-c3c(F)cccc3Cl)n2)c(N2C[C@@H](N)C[C@H]2CO)c1] N-(2-((2S,4S)-4-amino-2- (hydroxymethyl)pyrrolidin-1-yl)-4- (4-cyanopyridin-3-yl)phenyl)-1-(2- chloro-6-fluorophenyl)-6-oxo-1,6- dihydropyridazine-3-carboxamide | HCl base | LC-MS m/z: 560.2 (M + 1)+ 1H NMR (500 MHz, CD₃OD) δ 8.99 (s, 1H), 8.86-8.81 (m, 1H), 8.71-8.57 (m, 1H), 8.30 (dd, J = 5.1, 9.8 Hz, 1H), 8.02-7.95 (m, 1H), 7.94 (dd, J = 2.0, 7.8 Hz, 1H), 7.73-7.66 (m, 1H), 7.63-7.58 (m, 2H), 7.50-7.43 (m, 1H), 7.35 (dd, J = 1.6, 9.8 Hz, 1H), 3.87 (br s, 1H), 3.81-3.68 (m, 1H), 3.47 (br s, 1H), 3.46-3.35 (m, 3H), 2.74-2.64 (m, 1H), 2.01-1.95 (m, 1H). |
| Example 362 | 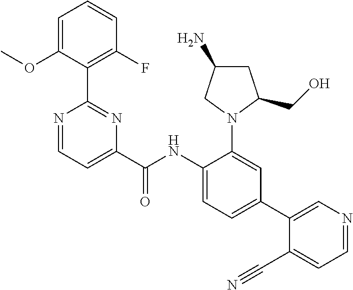 [SMILES: COc1cccc(F)c1-c1nccc(C(=O)Nc2ccc(-c3cnccc3C#N)cc2N2C[C@@H](N)C[C@H]2CO)n1] N-(2- ((2S,4S)-4-amino-2- (hydroxymethyl)pyrrolidin-1-yl)-4- (4-cyanopyridin-3-yl)phenyl)-2-(2- fluoro-6- methoxyphenyl)pyrimidine-4- carboxamide | HCl base | LC-MS m/z: 540.2 (M + 1)+ 1H NMR (400 MHz, CD₃OD) δ 9.20 (d, J = 5.1 Hz, 1H), 8.90 (d, J = 0.8 Hz, 1H), 8.79-8.70 (m, 2H), 8.27 (d, J = 5.1 Hz, 1H), 7.94 (d, J = 2.1 Hz, 1H), 7.84 (dd, J = 5.1, 0.8 Hz, 1H), 7.63-7.52 (m, 2H), 7.06 (d, J = 8.5 Hz, 1H), 7.01-6.92 (m, 1H), 3.83 (s, 3H), 3.82-3.77 (m, 2H), 3.45 (dt, J = 10.9, 2.9 Hz, 2H), 3.38 (ddd, J = 10.8, 5.1, 1.7 Hz, 2H), 2.70 (ddd, J = 14.3, 10.5, 6.2 Hz, 1H), 2.04-1.93 (m, 1H). |

### Example A: HPK1 Inhibition Assay

A stock solution of 10 mM test compound was prepared in DMSO. The compound was prepared by 5-fold and 10-point serial dilutions with HPK1 kinase assay buffer. The HPK1 kinase assay buffer contained 40 mM Tris, pH 7.5; 20 mM MgCl₂, 0.1 mg/ml BSA, and 50 μM DTT. 7.5 ng/μl of Active HPK1 (Promega, V4098) and 0.1 μg/ml MBP substrate protein were added to the reaction system. The final concentration of ATP in the HPK1 kinase reaction system was 10 μM. Serial diluted compounds were transferred to the reaction system and incubated at room temperature for 1 hr. 5 μL of ADP-Glo™ Reagent (Promega, V9101) was added and incubated for 40 min to stop the kinase reaction and deplete the unconsumed ATP. After that, 10 ul of kinase detection reagent was added and incubated at room temperature for 30-60 min. The luminescence was measured with a plate-reading luminometer.

Compounds of the present disclosure, as exemplified in Examples, showed the HPK1 IC₅₀ (nM) in the following ranges: A≤100 nM; B 100 nM<IC₅₀≤1000 nM.

| Compound | HPK1 IC₅₀ |
|---|---|
| Example 1 | B |
| Example 2 | A |
| Example 3 | B |
| Example 4 | B |
| Example 5 | A |
| Example 6 | B |
| Example 7 | A |
| Example 8 | A |
| Example 9 | B |
| Example 10 | A |
| Example 11 | A |
| Example 12 | A |
| Example 13 | A |
| Example 14 | A |
| Example 15 | A |
| Example 16 | B |
| Example 17 | A |
| Example 18 | A |
| Example 19 | A |
| Example 20 | A |
| Example 21 | A |
| Example 22 | A |
| Example 23 | A |
| Example 24 | A |
| Example 25 | A |
| Example 26 | A |
| Example 27 | A |
| Example 28 | B |
| Example 29 | B |
| Example 30 | B |
| Example 31 | A |
| Example 32 | B |
| Example 33 | B |
| Example 34 | A |
| Example 35 | A |
| Example 36 | A |
| Example 37 | A |
| Example 38 | A |
| Example 39 | B |
| Example 40 | A |
| Example 41 | A |
| Example 42 | A |
| Example 43 | A |
| Example 44 | A |
| Example 45 | A |
| Example 46 | A |
| Example 47 | B |
| Example 48 | B |
| Example 49 | A |
| Example 50 | A |
| Example 51 | A |
| Example 52 | B |
| Example 53 | B |
| Example 54 | A |
| Example 55 | A |
| Example 56 | A |
| Example 57 | A |
| Example 58 | A |
| Example 59 | A |
| Example 60 | A |
| Example 61 | A |
| Example 62 | B |
| Example 63 | A |
| Example 64 | B |
| Example 65 | B |
| Example 66 | A |
| Example 67 | A |
| Example 68 | B |
| Example 69 | B |
| Example 70 | A |
| Example 71 | A |
| Example 72 | A |
| Example 73 | A |
| Example 74 | A |
| Example 75 | A |
| Example 76 | A |
| Example 77 | A |
| Example 78 | A |
| Example 79 | A |
| Example 80 | A |
| Example 81 | A |
| Example 82 | A |
| Example 83 | A |
| Example 84 | B |
| Example 85 | A |
| Example 86 | B |
| Example 87 | B |
| Example 88 | A |
| Example 89 | A |
| Example 90 | A |
| Example 91 | A |
| Example 92 | A |
| Example 93 | A |
| Example 94 | A |
| Example 95 | A |
| Example 96 | A |
| Example 97 | A |
| Example 98 | A |
| Example 99 | A |
| Example 100 | A |
| Example 101 | A |
| Example 102 | A |
| Example 103 | A |
| Example 104 | A |
| Example 105 | A |
| Example 106 | A |
| Example 107 | B |
| Example 108 | A |
| Example 109 | A |
| Example 110 | A |
| Example 111 | A |
| Example 112 | A |
| Example 113 | A |
| Example 114 | A |
| Example 115 | A |
| Example 116 | A |
| Example 117 | A |
| Example 118 | A |
| Example 119 | A |
| Example 120 | A |
| Example 121 | A |
| Example 122 | A |
| Example 123 | A |
| Example 124 | A |
| Example 125 | A |
| Example 126 | A |
| Example 127 | A |
| Example 128 | A |
| Example 129 | A |
| Example 130 | A |
| Example 131 | A |
| Example 132 | A |
| Example 133 | A |
| Example 134 | A |
| Example 135 | A |
| Example 136 | A |
| Example 137 | A |
| Example 138 | A |
| Example 139 | A |
| Example 140 | A |
| Example 141 | A |
| Example 142 | A |
| Example 143 | A |
| Example 144 | A |
| Example 145 | A |
| Example 146 | A |
| Example 147 | A |
| Example 148 | A |
| Example 149 | A |
| Example 150 | A |
| Example 151 | A |
| Example 152 | A |
| Example 153 | A |
| Example 154 | A |
| Example 155 | A |
| Example 156 | A |
| Example 157 | A |
| Example 158 | A |
| Example 159 | A |
| Example 160 | A |
| Example 161 | A |
| Example 162 | A |
| Example 163 | A |
| Example 164 | A |
| Example 165 | A |
| Example 166 | A |
| Example 167 | A |
| Example 168 | A |
| Example 169 | A |
| Example 170 | A |
| Example 171 | A |
| Example 172 | A |
| Example 173 | A |
| Example 174 | A |
| Example 175 | A |
| Example 176 | A |
| Example 177 | A |
| Example 178 | A |
| Example 179 | A |
| Example 180 | A |
| Example 181 | A |
| Example 182 | A |
| Example 183 | A |
| Example 184 | A |
| Example 185 | A |
| Example 186 | A |
| Example 187 | A |
| Example 188 | A |
| Example 189 | A |
| Example 190 | A |
| Example 191 | A |
| Example 192 | A |
| Example 193 | A |
| Example 194 | A |
| Example 195 | A |
| Example 196 | A |
| Example 197 | A |
| Example 198 | A |
| Example 199 | B |
| Example 200 | A |
| Example 201 | A |
| Example 202 | A |
| Example 203 | A |
| Example 204 | A |
| Example 205 | A |
| Example 206 | A |
| Example 207 | A |
| Example 208 | A |
| Example 209 | A |
| Example 210 | A |
| Example 211 | A |
| Example 212 | A |
| Example 213 | A |
| Example 214 | A |
| Example 215 | A |
| Example 216 | A |
| Example 217 | A |
| Example 218 | A |
| Example 219 | A |
| Example 220 | A |
| Example 221 | A |
| Example 222 | A |
| Example 223 | A |
| Example 224 | A |
| Example 225 | A |
| Example 226 | A |
| Example 227 | A |
| Example 228 | A |
| Example 229 | A |
| Example 230 | A |
| Example 231 | A |
| Example 232 | A |
| Example 233 | A |
| Example 234 | A |
| Example 235 | A |
| Example 236 | A |
| Example 237 | A |
| Example 238 | A |
| Example 239 | A |
| Example 240 | A |
| Example 241 | A |
| Example 242 | A |
| Example 243 | A |
| Example 244 | A |
| Example 245 | A |
| Example 246 | A |
| Example 247 | A |
| Example 248 | A |
| Example 249 | A |
| Example 250 | A |
| Example 251 | A |
| Example 252 | A |
| Example 253 | A |
| Example 244 | A |
| Example 255 | A |
| Example 256 | A |
| Example 257 | A |
| Example 258 | A |
| Example 259 | A |
| Example 260 | A |
| Example 261 | A |
| Example 262 | A |
| Example 263 | A |
| Example 264 | A |
| Example 265 | A |
| Example 266 | A |
| Example 267 | A |
| Example 268 | A |
| Example 269 | A |
| Example 270 | A |
| Example 271 | A |
| Example 272 | A |
| Example 273 | A |
| Example 274 | A |
| Example 275 | A |
| Example 276 | A |
| Example 277 | A |
| Example 278 | A |
| Example 279 | A |
| Example 280 | A |
| Example 281 | A |
| Example 282 | A |
| Example 283 | A |
| Example 284 | A |
| Example 285 | A |
| Example 286 | A |
| Example 287 | A |
| Example 288 | A |
| Example 289 | A |
| Example 290 | A |
| Example 291 | A |
| Example 292 | A |
| Example 293 | A |
| Example 294 | A |
| Example 295 | A |
| Example 296 | A |
| Example 297 | A |
| Example 298 | A |
| Example 299 | A |
| Example 290 | A |
| Example 291 | A |
| Example 292 | A |
| Example 293 | A |
| Example 294 | A |
| Example 295 | A |
| Example 296 | A |
| Example 297 | A |
| Example 298 | A |
| Example 299 | A |
| Example 300 | A |
| Example 301 | A |
| Example 302 | A |
| Example 303 | A |
| Example 304 | A |
| Example 305 | A |
| Example 306 | A |
| Example 307 | A |
| Example 308 | A |
| Example 309 | A |
| Example 310 | A |
| Example 311 | A |
| Example 312 | A |
| Example 313 | A |
| Example 314 | A |
| Example 315 | A |
| Example 316 | A |
| Example 317 | A |
| Example 318 | A |
| Example 319 | A |
| Example 320 | A |
| Example 321 | A |
| Example 322 | A |
| Example 323 | A |
| Example 324 | A |
| Example 325 | A |
| Example 326 | A |
| Example 327 | A |
| Example 328 | A |
| Example 329 | A |
| Example 330 | A |
| Example 331 | A |
| Example 332 | A |
| Example 333 | A |
| Example 334 | A |
| Example 335 | A |
| Example 336 | A |
| Example 337 | A |
| Example 338 | A |
| Example 339 | A |
| Example 340 | A |
| Example 341 | A |
| Example 342 | A |
| Example 343 | A |
| Example 344 | A |
| Example 345 | A |
| Example 346 | A |
| Example 347 | A |
| Example 348 | A |
| Example 349 | A |
| Example 350 | A |
| Example 351 | A |
| Example 352 | A |
| Example 353 | A |
| Example 354 | A |
| Example 355 | A |
| Example 356 | A |
| Example 357 | A |
| Example 358 | A |
| Example 359 | A |
| Example 360 | A |
| Example 361 | A |
| Example 362 | A |

1. A compound of Formula (I) or a stereoisomer or pharmaceutically acceptable salt thereof: 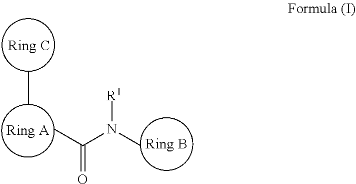
wherein
R¹ is H, alkyl, cycloalkyl, or substituted alkyl;
ring A is a 5- or 6-membered aryl or heteroaryl, and is optionally substituted with one or more substituents each of which is independently alkyl, halo, or haloalkyl; wherein the 5- or 6-membered heteroaryl has at least one ring-forming carbon atom and 1, 2, or 3 ring-forming heteroatoms each of which is independently N, O, or S; a ring-forming N atom is optionally substituted by oxo to form N⁺—O⁻; a ring-forming carbon atom of the 5 or 6-membered aryl or heteroaryl is optionally substituted by oxo to form a carbonyl group;
ring B is a 5- or 6-membered monocyclic aryl or heteroaryl, or a 8- to 12-membered fused bicyclic aryl or heteroaryl; Ring B is optionally substituted with 1-5 substituents each of which is independently alkyl, haloalkyl, halo, hydroxy, hydroxyalkyl, alkoxy, alkoxyalkyl, alkthioalkyl, alk-S(═O)₂-alkyl, cyano, hydroxyalkamino, amido, amino, alkamino, —NR¹⁰R¹¹, cycloalkyl, alk-cycloalkyl, heterocycloalkyl, alk-heterocycloalkyl, aryl, heteroaryl, bridged bicyclic cycloalkyl or heterocyloalkyl, fused bicyclic cycloalkyl or heterocyloalkyl, spirocyclic cycloalkyl or heterocyloalkyl, or multicycloalkyl; a ring carbon atom in ring B is optionally substituted with oxo to form a carbonyl group;
wherein each of R¹⁰ and R¹¹ is independently H, alkyl, halo, haloalkyl, cycloalkyl, alk-cycloalkyl, alk-heterocycloalkyl, or heterocycloalkyl;
ring C is a 5- or 6-membered aryl or heteroaryl and is optionally substituted with one or more substituents each of which is independently alkyl, haloalkyl, halo, hydroxy, hydroxyalkyl, hydroxyalkamino, alkoxy, alkoxyalkyl, amino, alkylamino, dialkylamino, amido, alkamino, cyano, cycloalkyl, alk-cycloalkyl, or heterocycloalkyl; and the heteroaryl has at least one ring carbon atom and 1, 2 or 3 ring heteroatoms each of which being independently N, O, or S.

2. The compound of claim 1, wherein R¹ is H.

3. The compound of claim 1, wherein ring A is 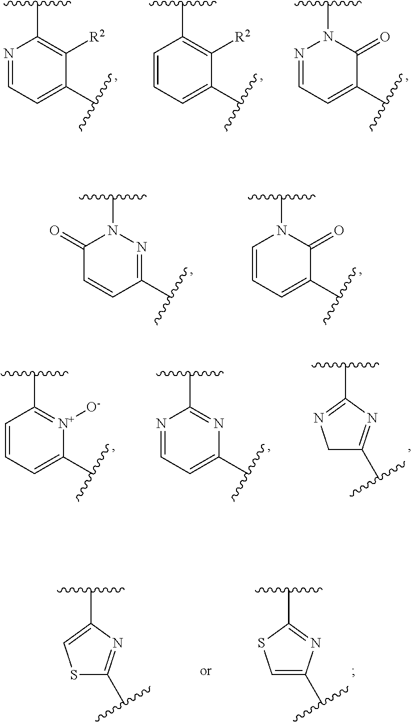
and R² is H, alkyl, halo or haloalkyl.

4. The compound of claim 3, wherein R² is H or halo.

5. The compound of claim 4, wherein halo is —F.

6. The compound of claim 1, wherein ring B is 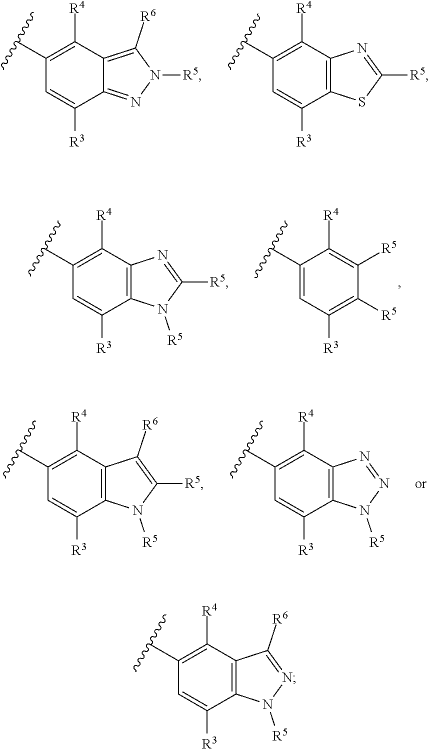
R³ is H, halo, haloalkyl or alkyl;
R⁴ is cycloalkyl, heterocycloalkyl, bridged bicyclic cycloalkyl or heterocyloalkyl, fused bicyclic cycloalkyl or heterocyloalkyl, spirocyclic cycloalkyl or heterocyloalkyl, multicycloalkyl, or —NR¹⁰R¹¹; wherein each substituent is optionally further substituted with one or more substituents; wherein R¹⁰ and R¹¹ are each independently selected from H, alkyl, halo, haloalkyl, cycloalkyl, alk-cycloalkyl, alk-heterocycloalkyl or heterocycloalkyl;
each R⁵ is independently H, alkyl, CD₃, alkthioalkyl, alk-S(═O)₂-alkyl, halo, haloalkyl, alkoxy, alkoxyalkyl, alkthioalkyl, alk-S(═O)₂-alkyl, cyano, amino, hydroxyl, hydroxyalkyl, hydroxyalkamino, amido, alkamino, cycloalkyl, alk-cycloalkyl, heterocyloalkyl, alk-heterocycloalkyl, fused cycloalkyl, bridged cycloalkyl, spirocycloalkyl, multicycloalkyl, aryl, or heteroaryl, wherein each substituent is optionally further substituted; wherein any hydrogen of alkyl can be optionally replaced with D; and
R⁶ is H, halo, haloalkyl, or alkyl.

7. The compound of claim 6, wherein R³ is H or F.

8. The compound of claim 6, wherein R⁶ is H or F.

9. The compound of claim 6, wherein R⁴ is cycloalkyl, heterocycloalkyl, bridged bicyclic cycloalkyl or heterocyloalkyl, fused bicyclic cycloalkyl or heterocyloalkyl, spirocyclic cycloalkyl or heterocyloalkyl, or multicycloalkyl, each of which is optionally further substituted with one or more substituents selected from hydroxyl, hydroxyalkyl, hydroxyalkylamino, halo, haloalkyl, alkyl, amino, alkylamino, dialkylamino,-alkamino, alkoxyl, alkoxyalkyl, —NH—C(═O)—(CH₂)ₙ—NH₂, amido, cycloalkyl, alk-cycloalkyl, heterocycloalkyl or alk-heterocycloalkyl; and n is 0, 1, 2, or 3.

10. The compound of claim 1, wherein ring C is phenyl, substituted with one or more substituents, each of which is independently —F, —Cl, or alkoxy.

11. The compound of claim 10, wherein ring C is

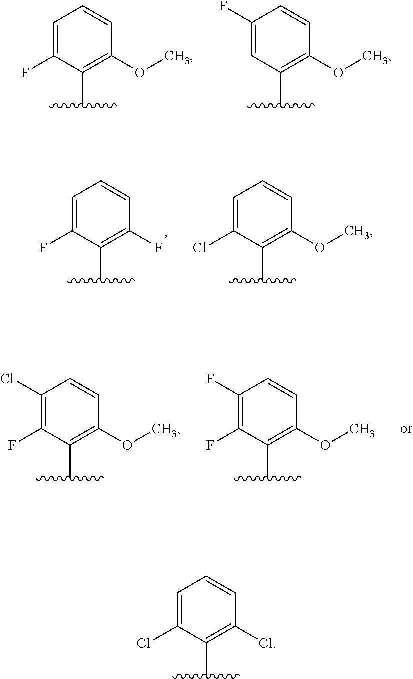

12. The compound of claim 1, wherein the compound is of Formula (Ia) or Formula (Ib), or a stereoisomer, or pharmaceutically acceptable salt thereof, 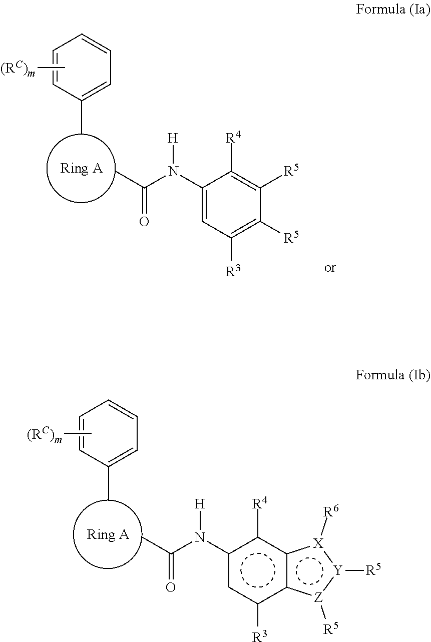
wherein ring A is 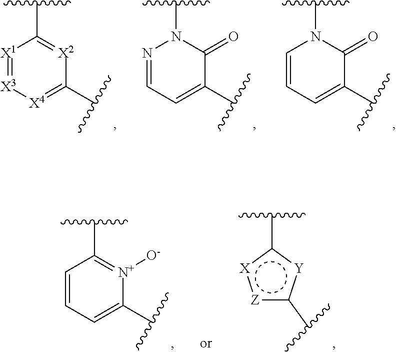
and is
optionally further substituted with one or more R²
each of X¹, X², X³, and X⁴ is independently a carbon atom (C) or N, and optionally substituted with R²; R² is H, alkyl, halo or haloalkyl;
each R^{c} is independently alkyl, haloalkyl, halo, hydroxy, hydroxyalkyl, hydroxyalkamino, alkoxy, alkoxyalkyl, amino, alkylamino, dialkylamino, amido, alkamino, cyano, cycloalkyl, alk-cycloalkyl, or heterocycloalkyl;
m is 0, 1, 2, 3, or 4;
each of X, Y and Z is independently a carbon atom or heteroatom selected from N or S;
dashed bond represents a single bond or a double bond as required to complete the valencies of the atoms being linked by the bond;
R³, R⁴, R⁵, and R⁶ are the same as of claim 6.

13. The compound of claim 1, wherein the compound is

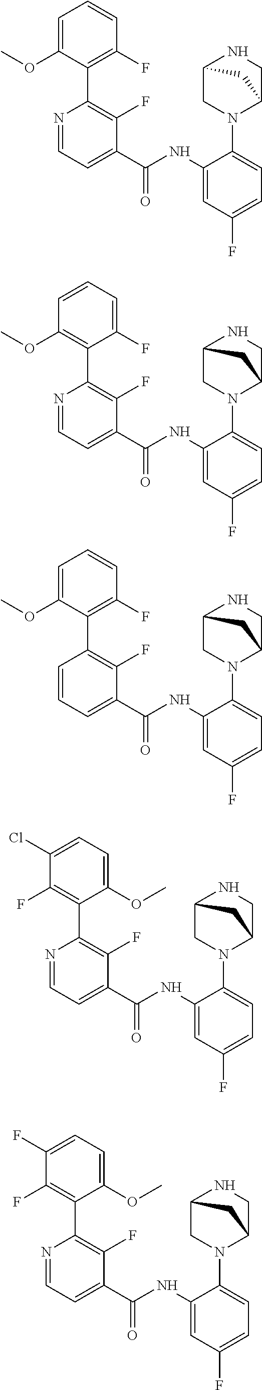 [SMILES: COc1cccc(F)c1-c1nccc(C(=O)Nc2cc(F)ccc2N2C[C@@H]3C[C@H]2CN3)c1F ; COc1cccc(F)c1-c1nccc(C(=O)Nc2cc(F)ccc2N2C[C@H]3C[C@@H]2CN3)c1F ; COc1cccc(F)c1-c1cccc(C(=O)Nc2cc(F)ccc2N2C[C@H]3C[C@@H]2CN3)c1F ; COc1ccc(Cl)c(F)c1-c1nccc(C(=O)Nc2cc(F)ccc2N2C[C@H]3C[C@@H]2CN3)c1F ; COc1ccc(F)c(F)c1-c1nccc(C(=O)Nc2cc(F)ccc2N2C[C@H]3C[C@@H]2CN3)c1F]

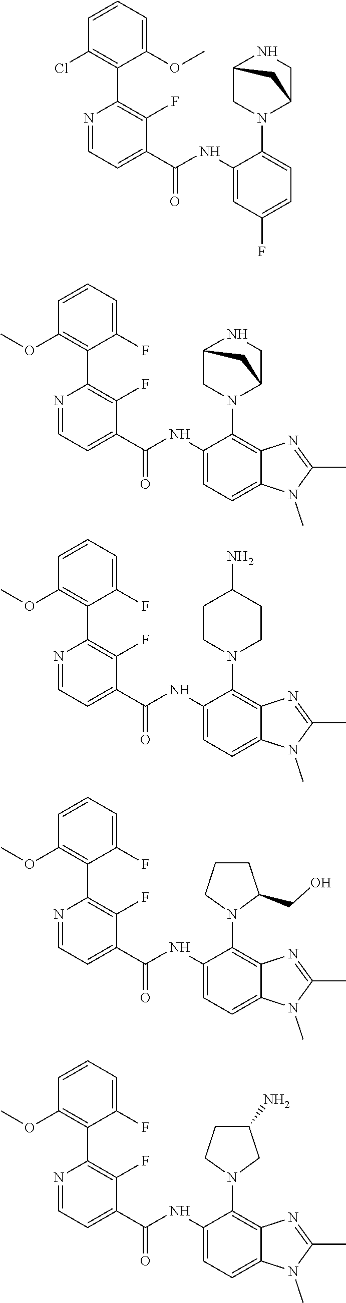

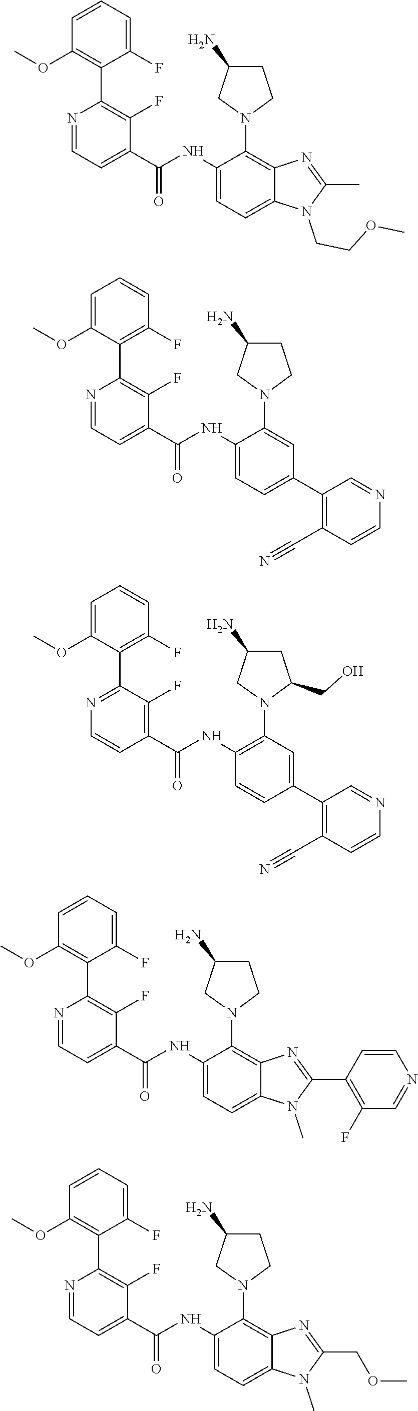

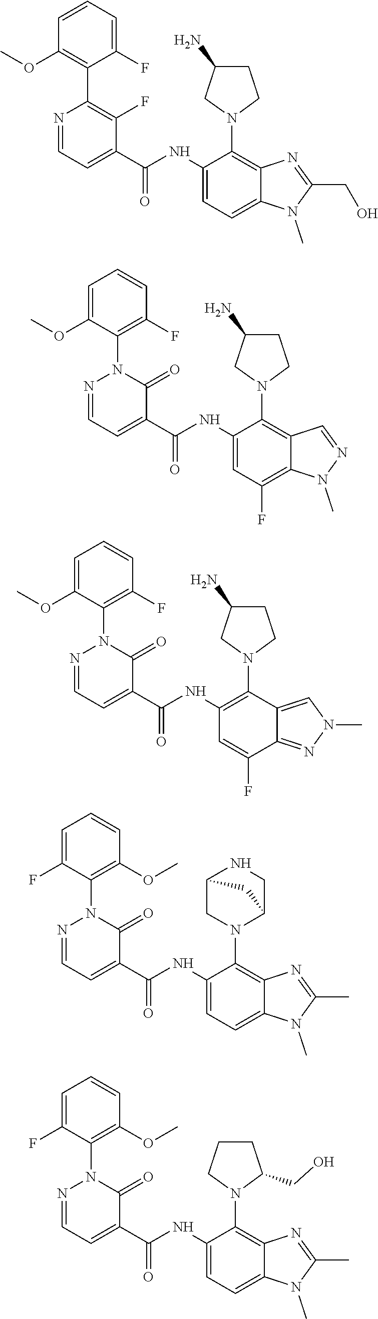

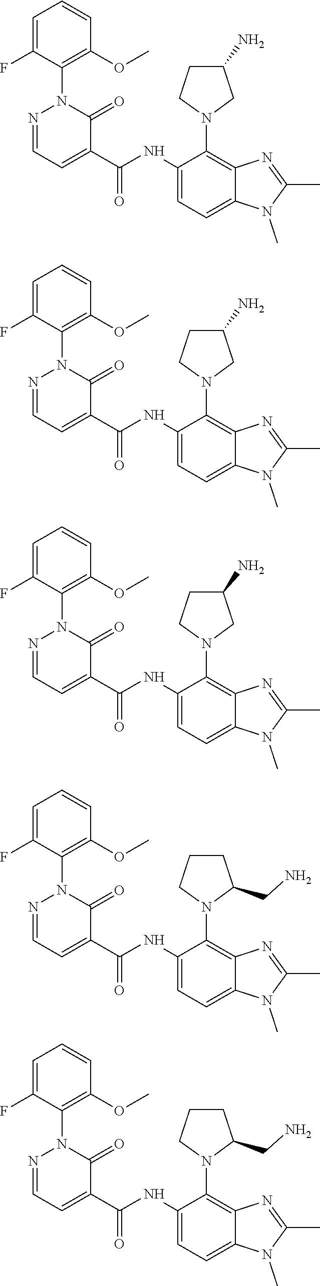 [SMILES: COc1cccc(F)c1-n1nccc(C(=O)Nc2ccc3c(nc(C)n3C)c2N2CC[C@H](N)C2)c1=O ; COc1cccc(F)c1-n1nccc(C(=O)Nc2ccc3c(nc(C)n3C)c2N2CC[C@H](N)C2)c1=O ; COc1cccc(F)c1-n1nccc(C(=O)Nc2ccc3c(nc(C)n3C)c2N2CC[C@@H](N)C2)c1=O ; COc1cccc(F)c1-n1nccc(C(=O)Nc2ccc3c(nc(C)n3C)c2N2CCC[C@H]2CN)c1=O ; COc1cccc(F)c1-n1nccc(C(=O)Nc2ccc3c(nc(C)n3C)c2N2CCC[C@H]2CN)c1=O]

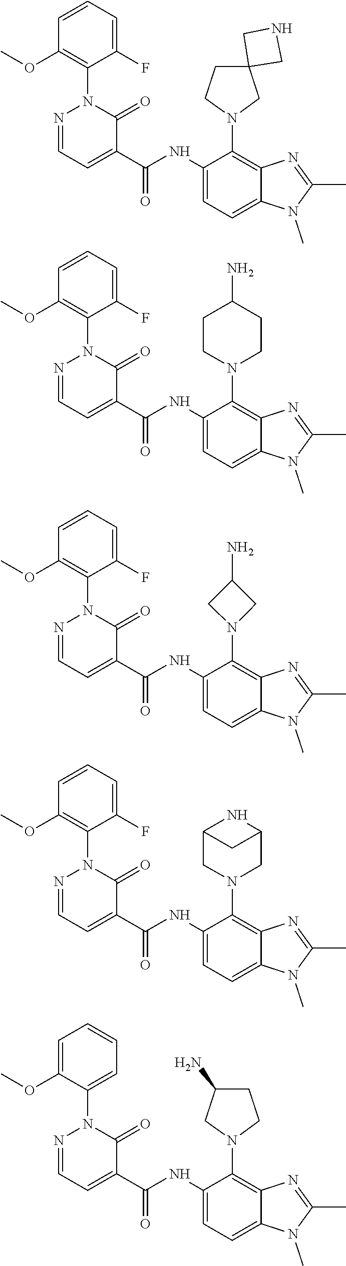

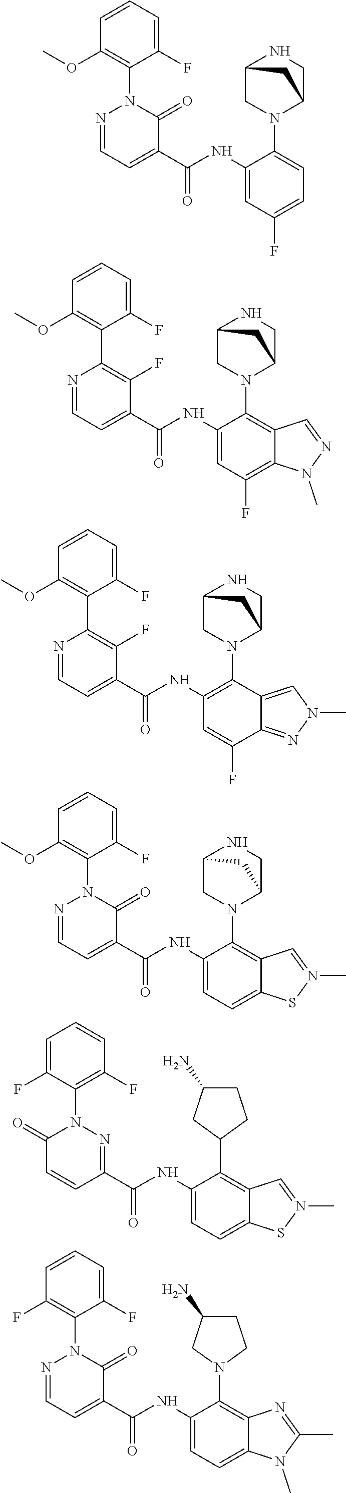

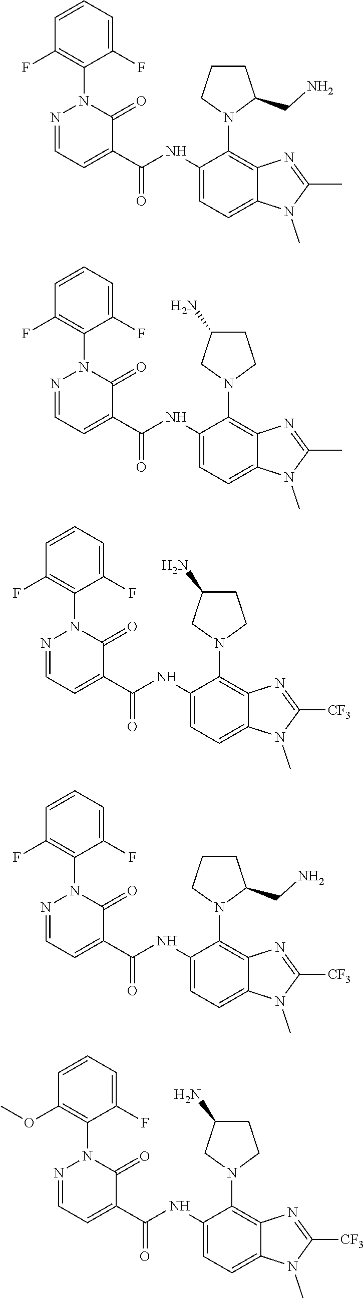

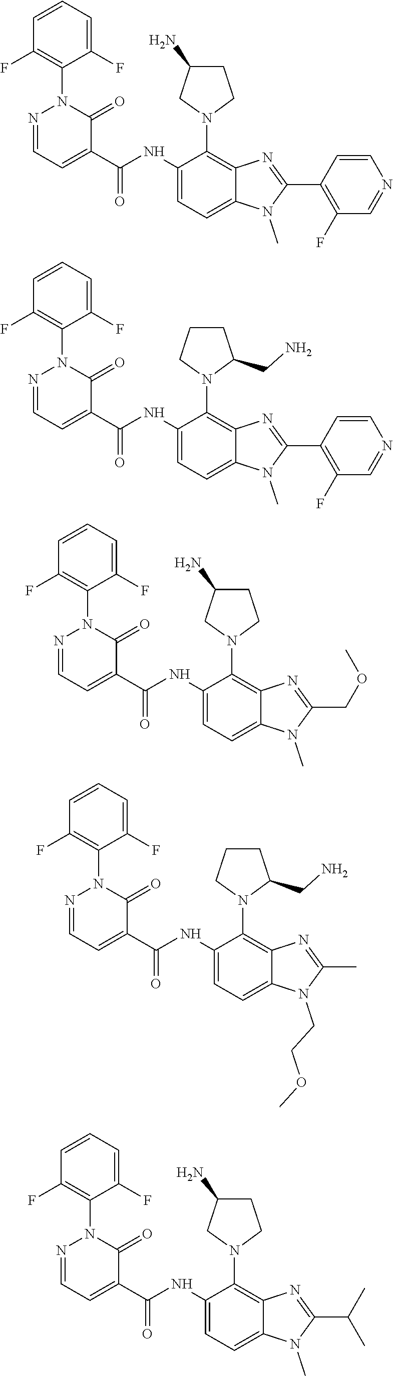 [SMILES: Cn1c(-c2ccncc2F)nc2c(N3CC[C@H](N)C3)c(NC(=O)c3ccnn(-c4c(F)cccc4F)c3=O)ccc21 ; Cn1c(-c2ccncc2F)nc2c(N3CCC[C@H]3CN)c(NC(=O)c3ccnn(-c4c(F)cccc4F)c3=O)ccc21 ; COCc1nc2c(N3CC[C@H](N)C3)c(NC(=O)c3ccnn(-c4c(F)cccc4F)c3=O)ccc2n1C ; COCCn1c(C)nc2c(N3CCC[C@H]3CN)c(NC(=O)c3ccnn(-c4c(F)cccc4F)c3=O)ccc21 ; CC(C)c1nc2c(N3CC[C@H](N)C3)c(NC(=O)c3ccnn(-c4c(F)cccc4F)c3=O)ccc2n1C]

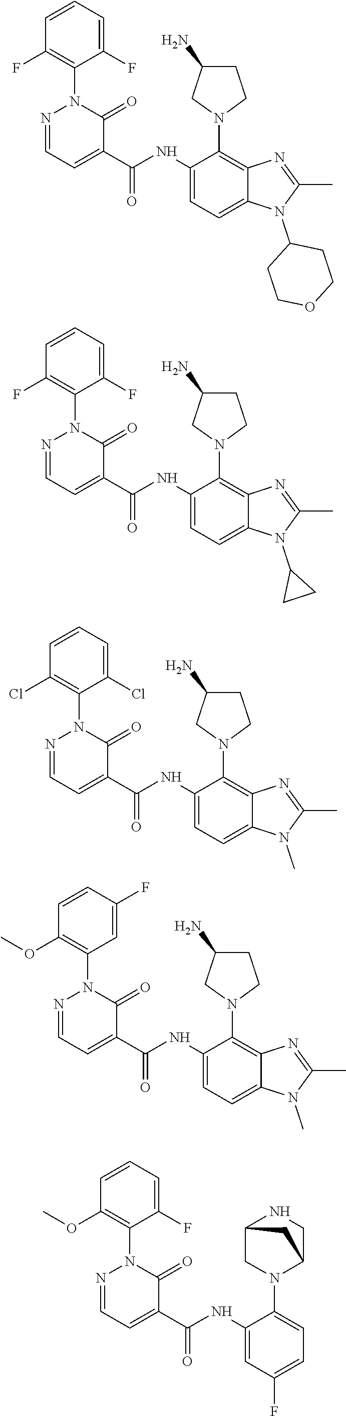

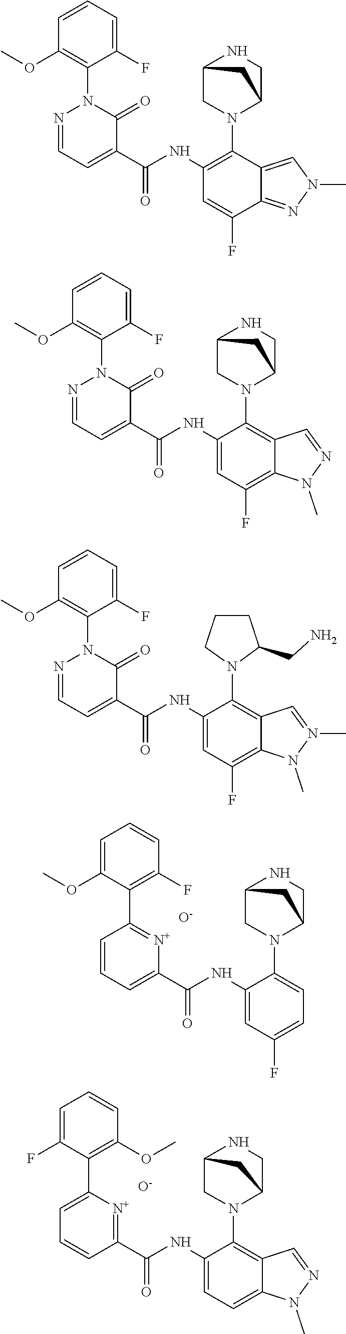

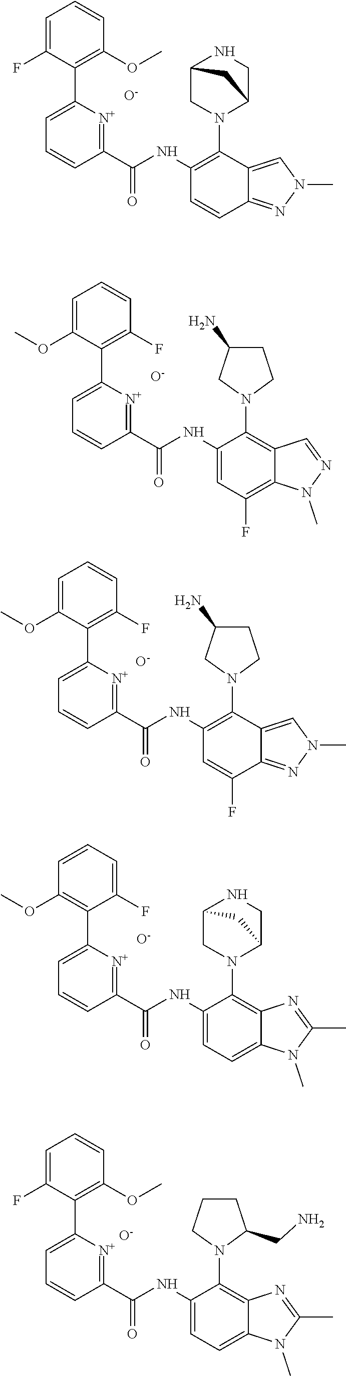

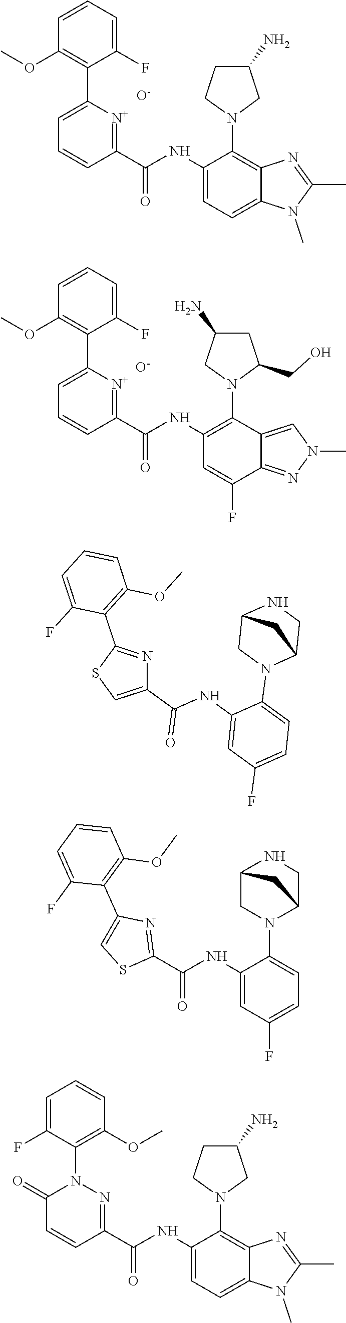

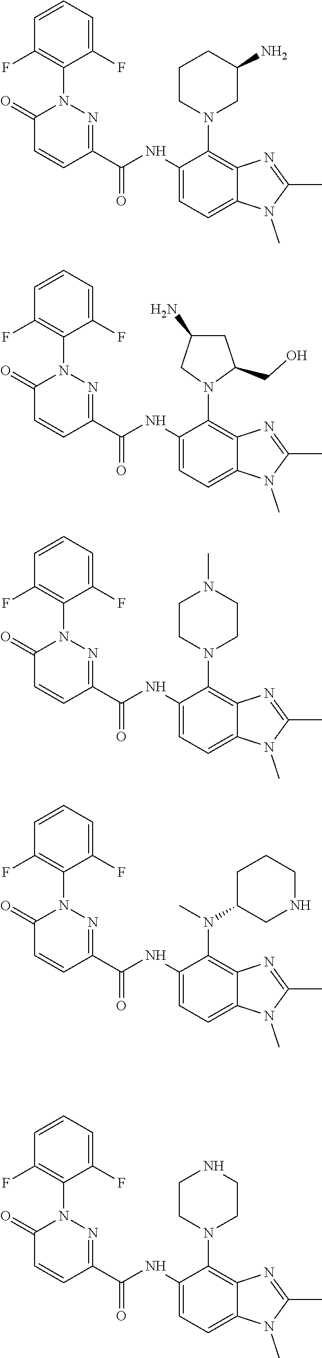 [SMILES: Cc1nc2c(N3CCC[C@@H](N)C3)c(NC(=O)c3ccc(=O)n(-c4c(F)cccc4F)n3)ccc2n1C ; Cc1nc2c(N3C[C@@H](N)C[C@H]3CO)c(NC(=O)c3ccc(=O)n(-c4c(F)cccc4F)n3)ccc2n1C ; Cc1nc2c(N3CCN(C)CC3)c(NC(=O)c3ccc(=O)n(-c4c(F)cccc4F)n3)ccc2n1C ; Cc1nc2c(N(C)[C@@H]3CCCNC3)c(NC(=O)c3ccc(=O)n(-c4c(F)cccc4F)n3)ccc2n1C ; Cc1nc2c(N3CCNCC3)c(NC(=O)c3ccc(=O)n(-c4c(F)cccc4F)n3)ccc2n1C]

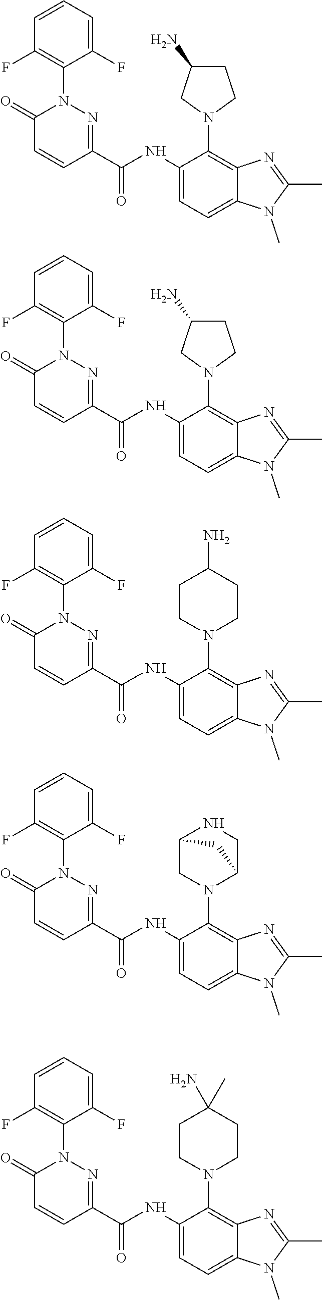 [SMILES: Cc1nc2c(N3CC[C@H](N)C3)c(NC(=O)c3ccc(=O)n(-c4c(F)cccc4F)n3)ccc2n1C ; Cc1nc2c(N3CC[C@@H](N)C3)c(NC(=O)c3ccc(=O)n(-c4c(F)cccc4F)n3)ccc2n1C ; Cc1nc2c(N3CCC(N)CC3)c(NC(=O)c3ccc(=O)n(-c4c(F)cccc4F)n3)ccc2n1C ; Cc1nc2c(N3C[C@@H]4C[C@H]3CN4)c(NC(=O)c3ccc(=O)n(-c4c(F)cccc4F)n3)ccc2n1C ; Cc1nc2c(N3CCC(C)(N)CC3)c(NC(=O)c3ccc(=O)n(-c4c(F)cccc4F)n3)ccc2n1C]

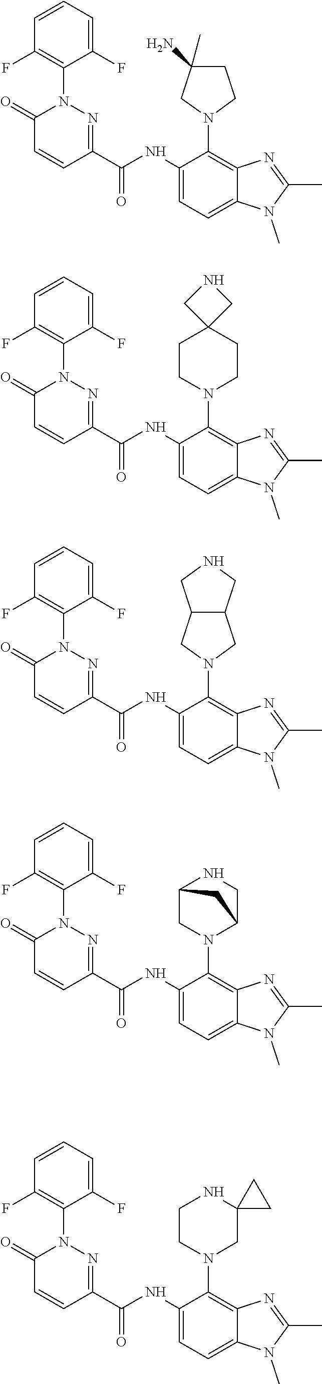 [SMILES: Cc1nc2c(N3CC[C@](C)(N)C3)c(NC(=O)c3ccc(=O)n(-c4c(F)cccc4F)n3)ccc2n1C ; Cc1nc2c(N3CCC4(CC3)CNC4)c(NC(=O)c3ccc(=O)n(-c4c(F)cccc4F)n3)ccc2n1C ; Cc1nc2c(N3CC4CNCC4C3)c(NC(=O)c3ccc(=O)n(-c4c(F)cccc4F)n3)ccc2n1C ; Cc1nc2c(N3C[C@H]4C[C@@H]3CN4)c(NC(=O)c3ccc(=O)n(-c4c(F)cccc4F)n3)ccc2n1C ; Cc1nc2c(N3CCNC4(CC4)C3)c(NC(=O)c3ccc(=O)n(-c4c(F)cccc4F)n3)ccc2n1C]

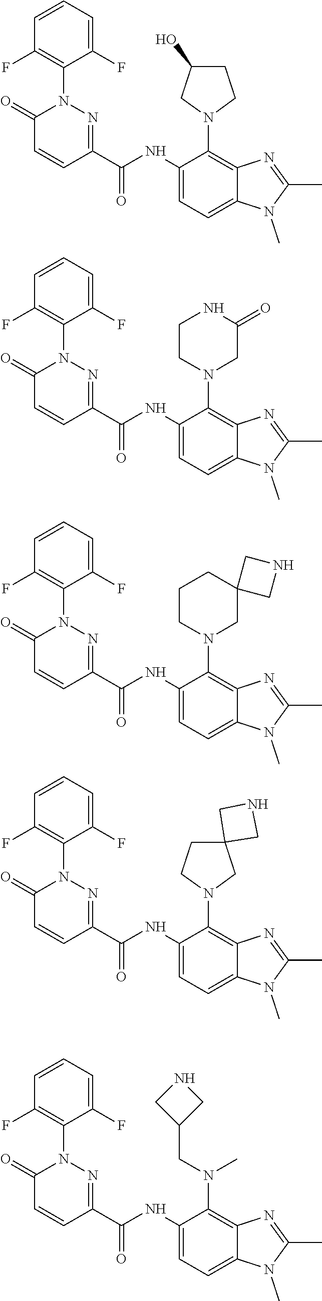 [SMILES: Cc1nc2c(N3CC[C@H](O)C3)c(NC(=O)c3ccc(=O)n(-c4c(F)cccc4F)n3)ccc2n1C ; Cc1nc2c(N3CCNC(=O)C3)c(NC(=O)c3ccc(=O)n(-c4c(F)cccc4F)n3)ccc2n1C ; Cc1nc2c(N3CCCC4(CNC4)C3)c(NC(=O)c3ccc(=O)n(-c4c(F)cccc4F)n3)ccc2n1C ; Cc1nc2c(N3CCC4(CNC4)C3)c(NC(=O)c3ccc(=O)n(-c4c(F)cccc4F)n3)ccc2n1C ; Cc1nc2c(N(C)CC3CNC3)c(NC(=O)c3ccc(=O)n(-c4c(F)cccc4F)n3)ccc2n1C]

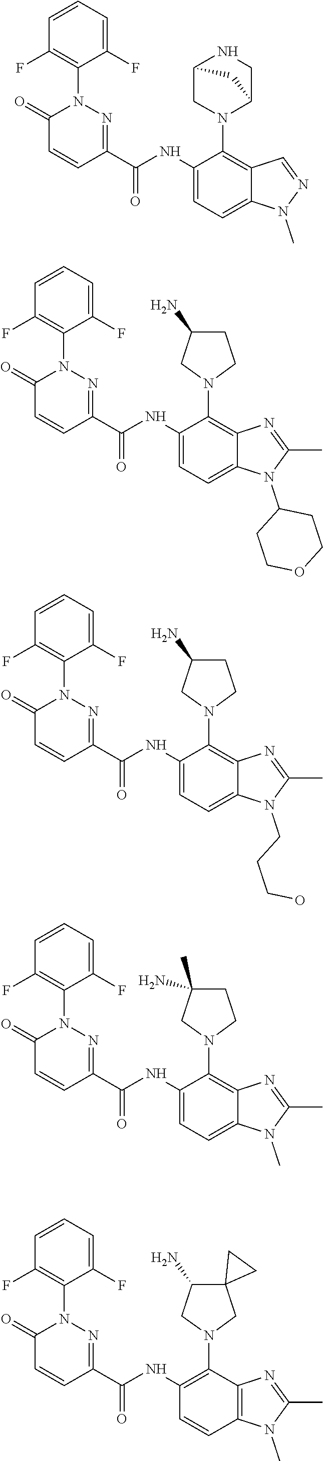 [SMILES: Cn1ncc2c(N3C[C@@H]4C[C@H]3CN4)c(NC(=O)c3ccc(=O)n(-c4c(F)cccc4F)n3)ccc21 ; Cc1nc2c(N3CC[C@H](N)C3)c(NC(=O)c3ccc(=O)n(-c4c(F)cccc4F)n3)ccc2n1C1CCOCC1 ; Cc1nc2c(N3CC[C@H](N)C3)c(NC(=O)c3ccc(=O)n(-c4c(F)cccc4F)n3)ccc2n1CCCO ; Cc1nc2c(N3CC[C@@](C)(N)C3)c(NC(=O)c3ccc(=O)n(-c4c(F)cccc4F)n3)ccc2n1C ; Cc1nc2c(N3C[C@H](N)C4(CC4)C3)c(NC(=O)c3ccc(=O)n(-c4c(F)cccc4F)n3)ccc2n1C]

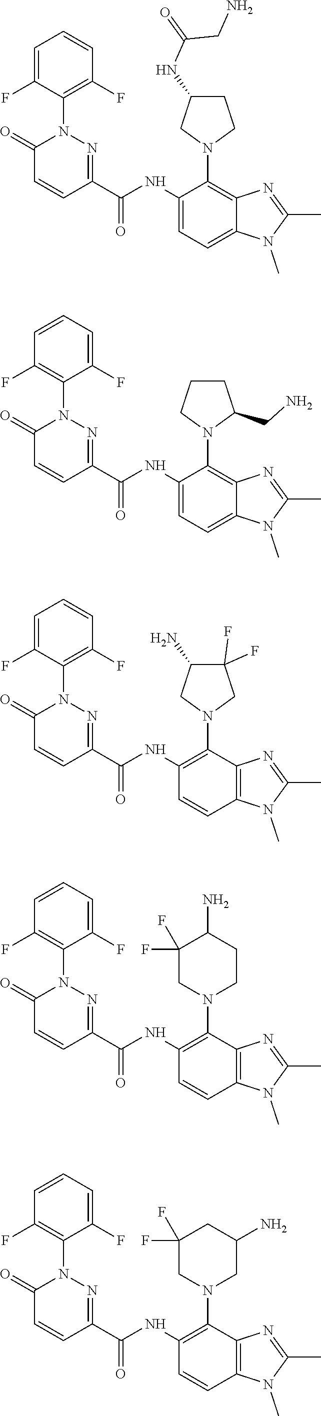 [SMILES: Cc1nc2c(N3CC[C@@H](NC(=O)CN)C3)c(NC(=O)c3ccc(=O)n(-c4c(F)cccc4F)n3)ccc2n1C ; Cc1nc2c(N3CCC[C@H]3CN)c(NC(=O)c3ccc(=O)n(-c4c(F)cccc4F)n3)ccc2n1C ; Cc1nc2c(N3C[C@H](N)C(F)(F)C3)c(NC(=O)c3ccc(=O)n(-c4c(F)cccc4F)n3)ccc2n1C ; Cc1nc2c(N3CCC(N)C(F)(F)C3)c(NC(=O)c3ccc(=O)n(-c4c(F)cccc4F)n3)ccc2n1C ; Cc1nc2c(N3CC(N)CC(F)(F)C3)c(NC(=O)c3ccc(=O)n(-c4c(F)cccc4F)n3)ccc2n1C]

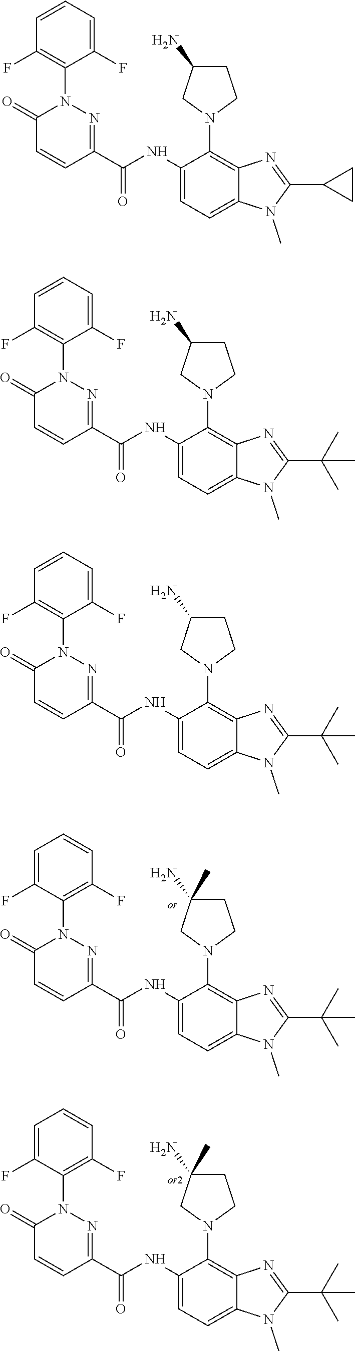 [SMILES: Cn1c(C2CC2)nc2c(N3CC[C@H](N)C3)c(NC(=O)c3ccc(=O)n(-c4c(F)cccc4F)n3)ccc21 ; Cn1c(C(C)(C)C)nc2c(N3CC[C@H](N)C3)c(NC(=O)c3ccc(=O)n(-c4c(F)cccc4F)n3)ccc21 ; Cn1c(C(C)(C)C)nc2c(N3CC[C@@H](N)C3)c(NC(=O)c3ccc(=O)n(-c4c(F)cccc4F)n3)ccc21 ; Cn1c(C(C)(C)C)nc2c(N3CC[C@@](C)(N)C3)c(NC(=O)c3ccc(=O)n(-c4c(F)cccc4F)n3)ccc21 ; Cn1c(C(C)(C)C)nc2c(N3CC[C@@](C)(N)C3)c(NC(=O)c3ccc(=O)n(-c4c(F)cccc4F)n3)ccc21]

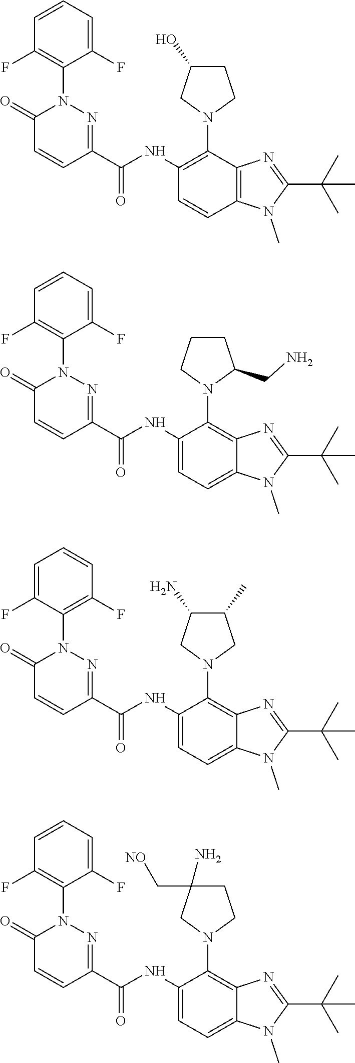

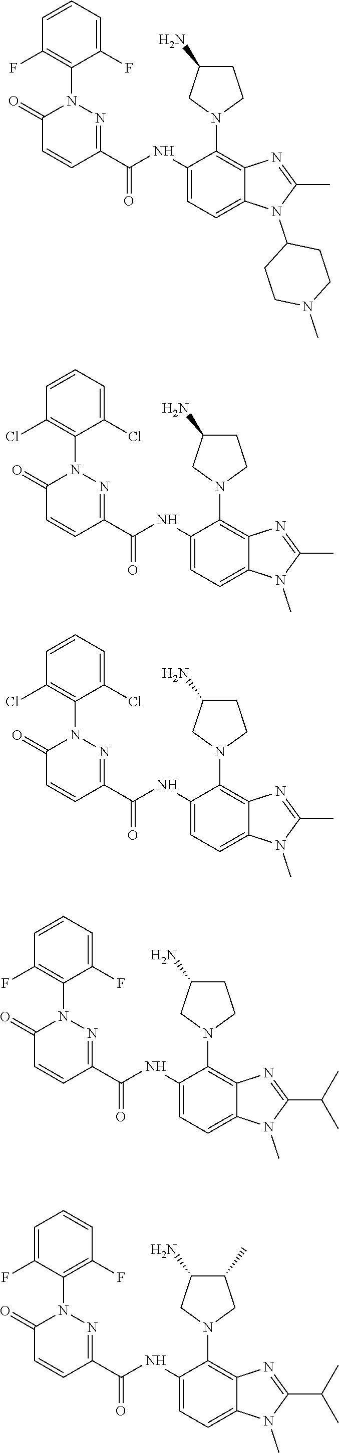 [SMILES: Cc1nc2c(N3CC[C@H](N)C3)c(NC(=O)c3ccc(=O)n(-c4c(F)cccc4F)n3)ccc2n1C1CCN(C)CC1 ; Cc1nc2c(N3CC[C@H](N)C3)c(NC(=O)c3ccc(=O)n(-c4c(Cl)cccc4Cl)n3)ccc2n1C ; Cc1nc2c(N3CC[C@@H](N)C3)c(NC(=O)c3ccc(=O)n(-c4c(Cl)cccc4Cl)n3)ccc2n1C ; CC(C)c1nc2c(N3CC[C@@H](N)C3)c(NC(=O)c3ccc(=O)n(-c4c(F)cccc4F)n3)ccc2n1C ; CC(C)c1nc2c(N3C[C@@H](C)[C@@H](N)C3)c(NC(=O)c3ccc(=O)n(-c4c(F)cccc4F)n3)ccc2n1C]

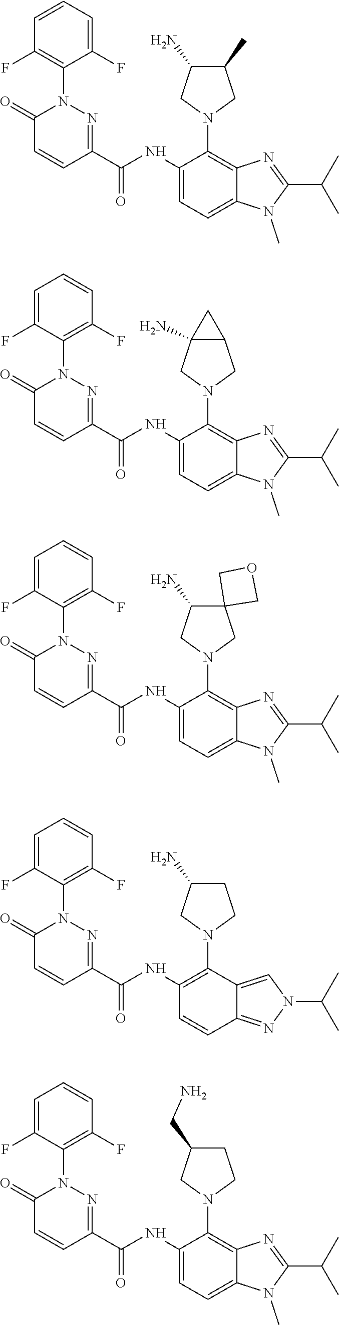 [SMILES: CC(C)c1nc2c(N3C[C@H](C)[C@@H](N)C3)c(NC(=O)c3ccc(=O)n(-c4c(F)cccc4F)n3)ccc2n1C ; CC(C)c1nc2c(N3CC4C[C@]4(N)C3)c(NC(=O)c3ccc(=O)n(-c4c(F)cccc4F)n3)ccc2n1C ; CC(C)c1nc2c(N3C[C@H](N)C4(COC4)C3)c(NC(=O)c3ccc(=O)n(-c4c(F)cccc4F)n3)ccc2n1C ; CC(C)n1cc2c(N3CC[C@@H](N)C3)c(NC(=O)c3ccc(=O)n(-c4c(F)cccc4F)n3)ccc2n1 ; CC(C)c1nc2c(N3CC[C@H](CN)C3)c(NC(=O)c3ccc(=O)n(-c4c(F)cccc4F)n3)ccc2n1C]

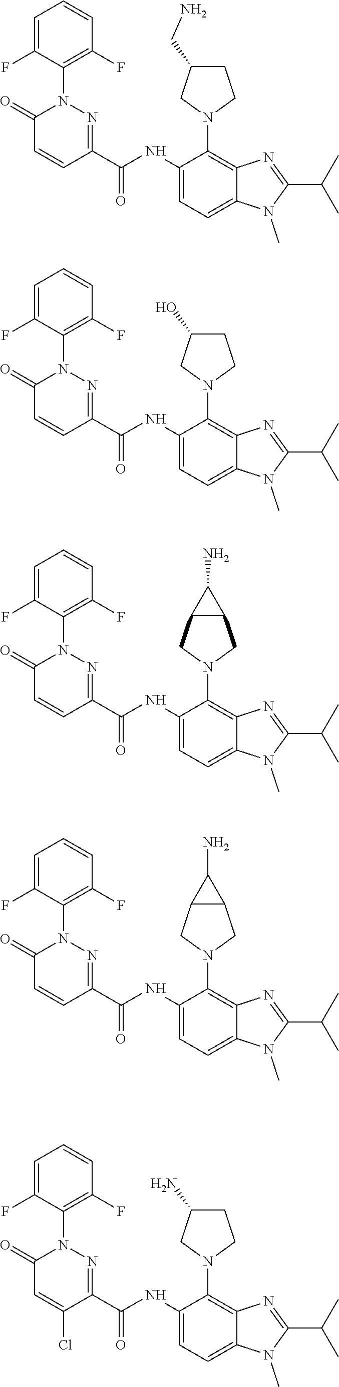

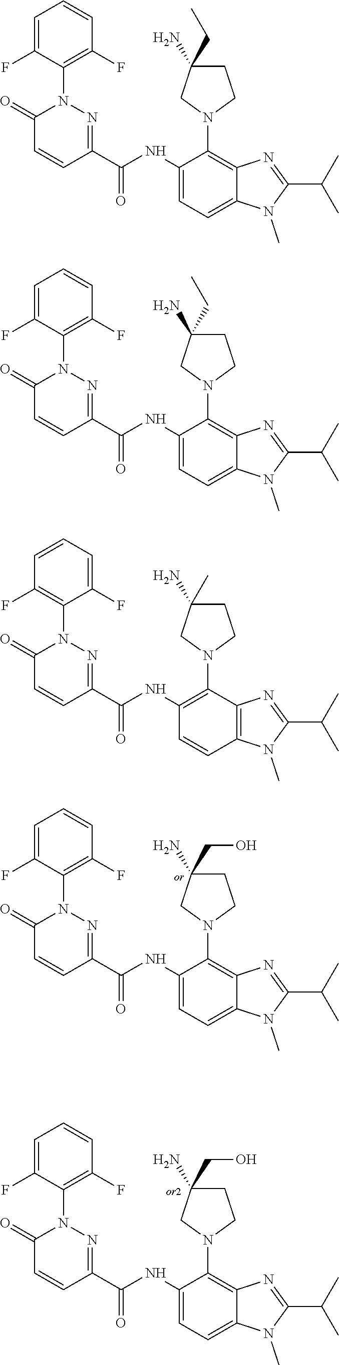 [SMILES: CC[C@@]1(N)CCN(c2c(NC(=O)c3ccc(=O)n(-c4c(F)cccc4F)n3)ccc3c2nc(C(C)C)n3C)C1 ; CC[C@]1(N)CCN(c2c(NC(=O)c3ccc(=O)n(-c4c(F)cccc4F)n3)ccc3c2nc(C(C)C)n3C)C1 ; CC(C)c1nc2c(N3CC[C@@](C)(N)C3)c(NC(=O)c3ccc(=O)n(-c4c(F)cccc4F)n3)ccc2n1C ; CC(C)c1nc2c(N3CC[C@](N)(CO)C3)c(NC(=O)c3ccc(=O)n(-c4c(F)cccc4F)n3)ccc2n1C ; CC(C)c1nc2c(N3CC[C@](N)(CO)C3)c(NC(=O)c3ccc(=O)n(-c4c(F)cccc4F)n3)ccc2n1C]

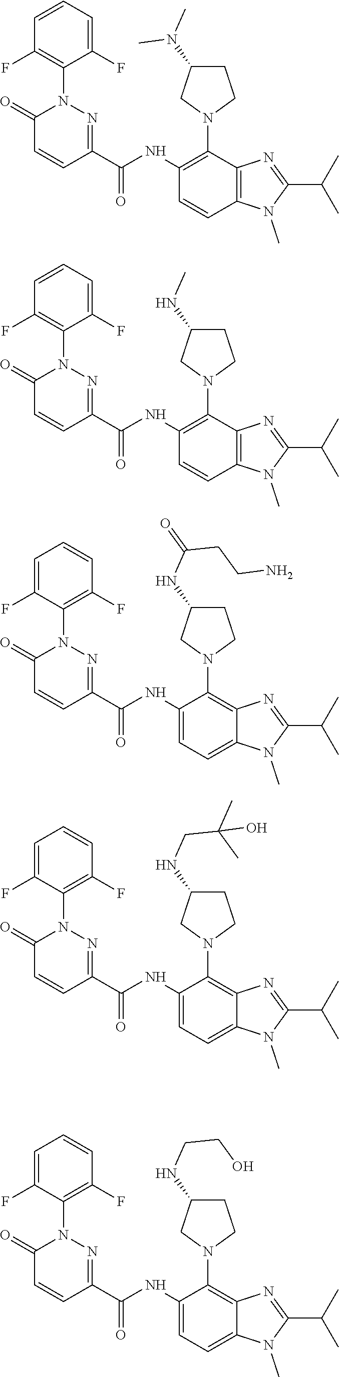 [SMILES: CC(C)c1nc2c(N3CC[C@@H](N(C)C)C3)c(NC(=O)c3ccc(=O)n(-c4c(F)cccc4F)n3)ccc2n1C ; CN[C@@H]1CCN(c2c(NC(=O)c3ccc(=O)n(-c4c(F)cccc4F)n3)ccc3c2nc(C(C)C)n3C)C1 ; CC(C)c1nc2c(N3CC[C@@H](NC(=O)CCN)C3)c(NC(=O)c3ccc(=O)n(-c4c(F)cccc4F)n3)ccc2n1C ; CC(C)c1nc2c(N3CC[C@@H](NCC(C)(C)O)C3)c(NC(=O)c3ccc(=O)n(-c4c(F)cccc4F)n3)ccc2n1C ; CC(C)c1nc2c(N3CC[C@@H](NCCO)C3)c(NC(=O)c3ccc(=O)n(-c4c(F)cccc4F)n3)ccc2n1C]

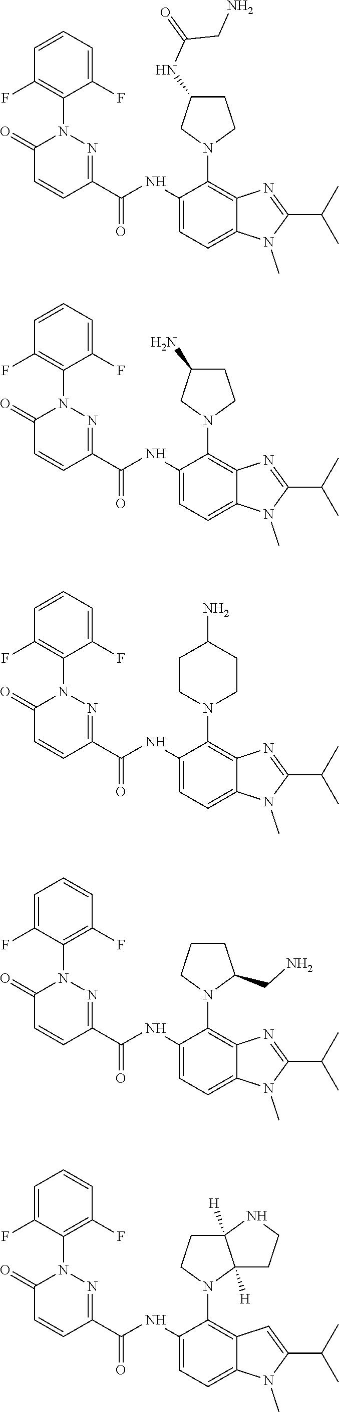 [SMILES: CC(C)c1nc2c(N3CC[C@@H](NC(=O)CN)C3)c(NC(=O)c3ccc(=O)n(-c4c(F)cccc4F)n3)ccc2n1C ; CC(C)c1nc2c(N3CC[C@H](N)C3)c(NC(=O)c3ccc(=O)n(-c4c(F)cccc4F)n3)ccc2n1C ; CC(C)c1nc2c(N3CCC(N)CC3)c(NC(=O)c3ccc(=O)n(-c4c(F)cccc4F)n3)ccc2n1C ; CC(C)c1nc2c(N3CCC[C@H]3CN)c(NC(=O)c3ccc(=O)n(-c4c(F)cccc4F)n3)ccc2n1C ; CC(C)c1cc2c(N3CC[C@H]4NCC[C@H]43)c(NC(=O)c3ccc(=O)n(-c4c(F)cccc4F)n3)ccc2n1C]

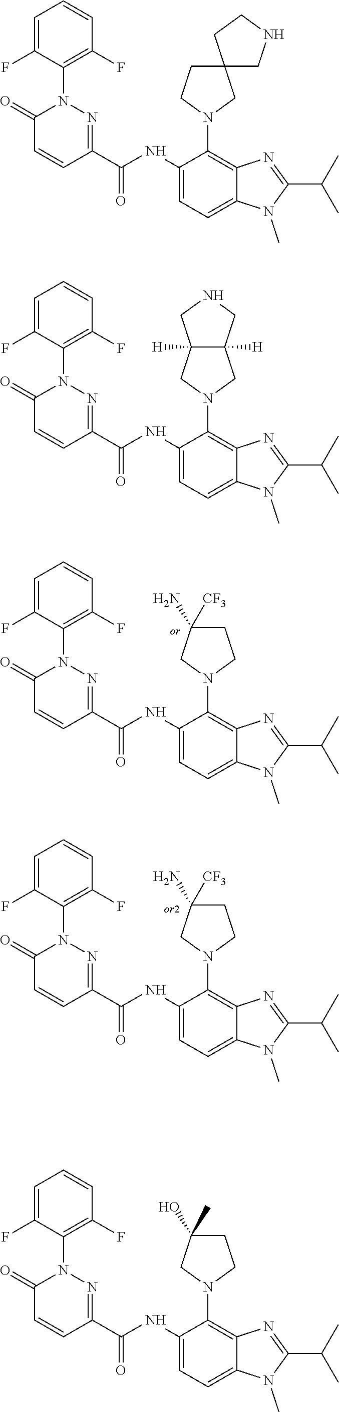 [SMILES: CC(C)c1nc2c(N3CCC4(CCNC4)C3)c(NC(=O)c3ccc(=O)n(-c4c(F)cccc4F)n3)ccc2n1C ; CC(C)c1nc2c(N3C[C@H]4CNC[C@H]4C3)c(NC(=O)c3ccc(=O)n(-c4c(F)cccc4F)n3)ccc2n1C ; CC(C)c1nc2c(N3CC[C@](N)(C(F)(F)F)C3)c(NC(=O)c3ccc(=O)n(-c4c(F)cccc4F)n3)ccc2n1C ; CC(C)c1nc2c(N3CC[C@](N)(C(F)(F)F)C3)c(NC(=O)c3ccc(=O)n(-c4c(F)cccc4F)n3)ccc2n1C ; CC(C)c1nc2c(N3CC[C@@](C)(O)C3)c(NC(=O)c3ccc(=O)n(-c4c(F)cccc4F)n3)ccc2n1C]

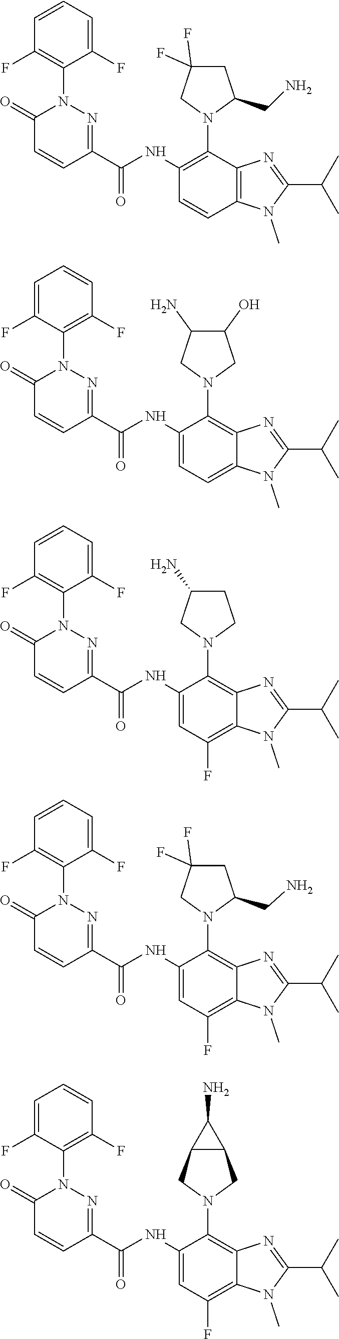

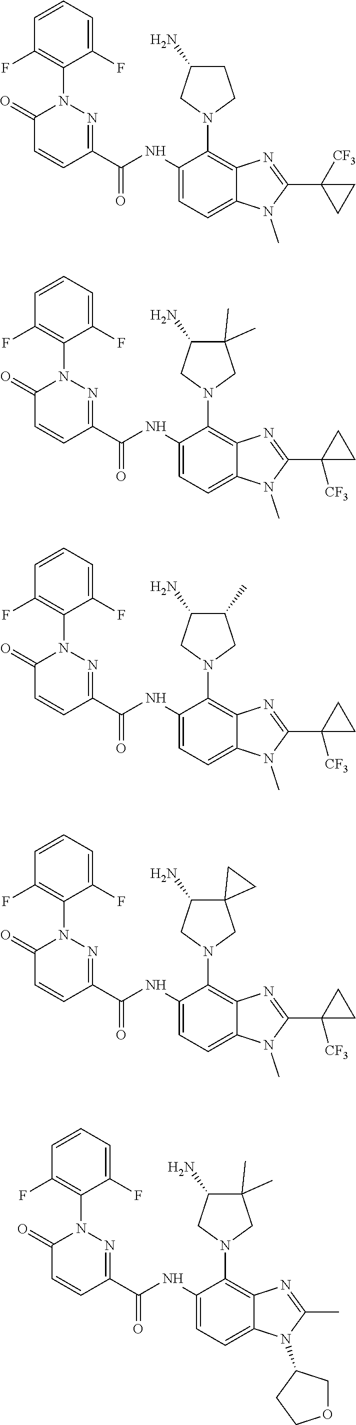 [SMILES: Cn1c(C2(C(F)(F)F)CC2)nc2c(N3CC[C@@H](N)C3)c(NC(=O)c3ccc(=O)n(-c4c(F)cccc4F)n3)ccc21 ; Cn1c(C2(C(F)(F)F)CC2)nc2c(N3C[C@H](N)C(C)(C)C3)c(NC(=O)c3ccc(=O)n(-c4c(F)cccc4F)n3)ccc21 ; C[C@@H]1CN(c2c(NC(=O)c3ccc(=O)n(-c4c(F)cccc4F)n3)ccc3c2nc(C2(C(F)(F)F)CC2)n3C)C[C@@H]1N ; Cn1c(C2(C(F)(F)F)CC2)nc2c(N3C[C@H](N)C4(CC4)C3)c(NC(=O)c3ccc(=O)n(-c4c(F)cccc4F)n3)ccc21 ; Cc1nc2c(N3C[C@H](N)C(C)(C)C3)c(NC(=O)c3ccc(=O)n(-c4c(F)cccc4F)n3)ccc2n1[C@H]1CCOC1]

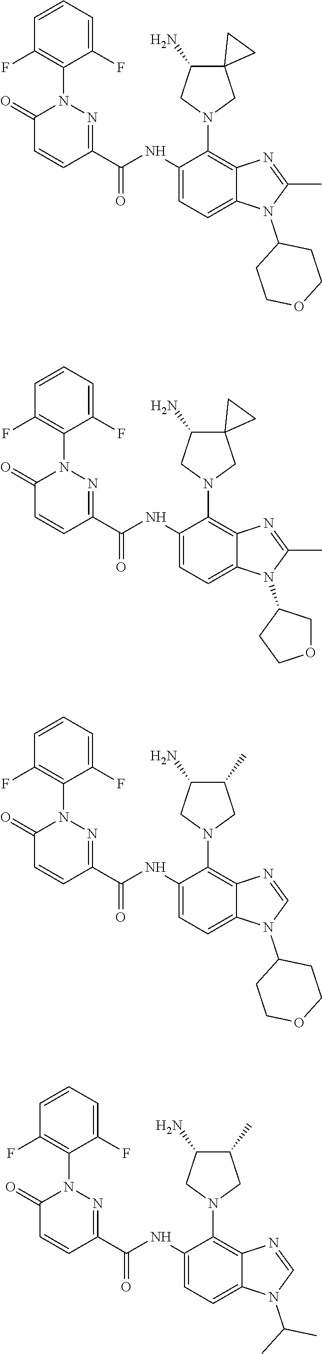 [SMILES: Cc1nc2c(N3C[C@H](N)C4(CC4)C3)c(NC(=O)c3ccc(=O)n(-c4c(F)cccc4F)n3)ccc2n1C1CCOCC1 ; Cc1nc2c(N3C[C@H](N)C4(CC4)C3)c(NC(=O)c3ccc(=O)n(-c4c(F)cccc4F)n3)ccc2n1[C@H]1CCOC1 ; C[C@@H]1CN(c2c(NC(=O)c3ccc(=O)n(-c4c(F)cccc4F)n3)ccc3c2ncn3C2CCOCC2)C[C@@H]1N ; CC(C)n1cnc2c(N3C[C@@H](C)[C@@H](N)C3)c(NC(=O)c3ccc(=O)n(-c4c(F)cccc4F)n3)ccc21]

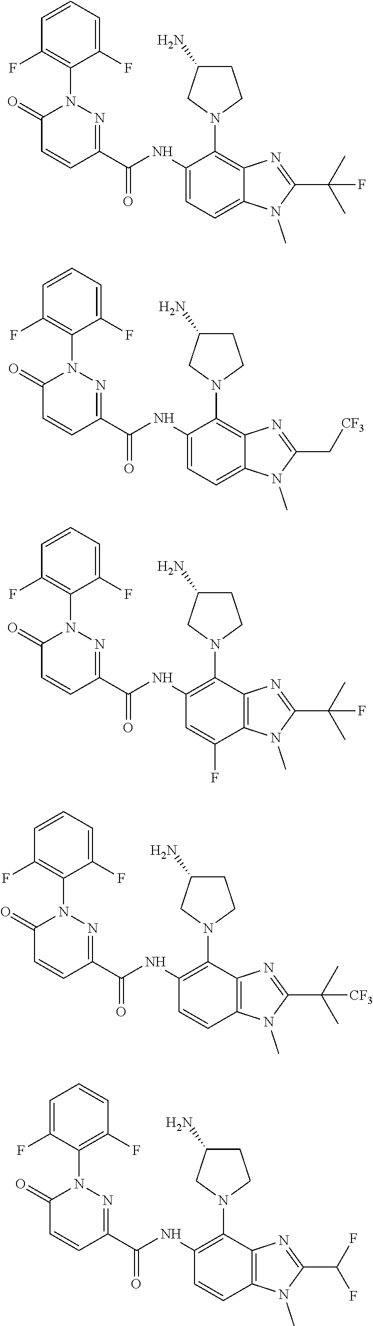 [SMILES: Cn1c(C(C)(C)F)nc2c(N3CC[C@@H](N)C3)c(NC(=O)c3ccc(=O)n(-c4c(F)cccc4F)n3)ccc21 ; Cn1c(CC(F)(F)F)nc2c(N3CC[C@@H](N)C3)c(NC(=O)c3ccc(=O)n(-c4c(F)cccc4F)n3)ccc21 ; Cn1c(C(C)(C)F)nc2c(N3CC[C@@H](N)C3)c(NC(=O)c3ccc(=O)n(-c4c(F)cccc4F)n3)cc(F)c21 ; Cn1c(C(C)(C)C(F)(F)F)nc2c(N3CC[C@@H](N)C3)c(NC(=O)c3ccc(=O)n(-c4c(F)cccc4F)n3)ccc21 ; Cn1c(C(F)F)nc2c(N3CC[C@@H](N)C3)c(NC(=O)c3ccc(=O)n(-c4c(F)cccc4F)n3)ccc21]

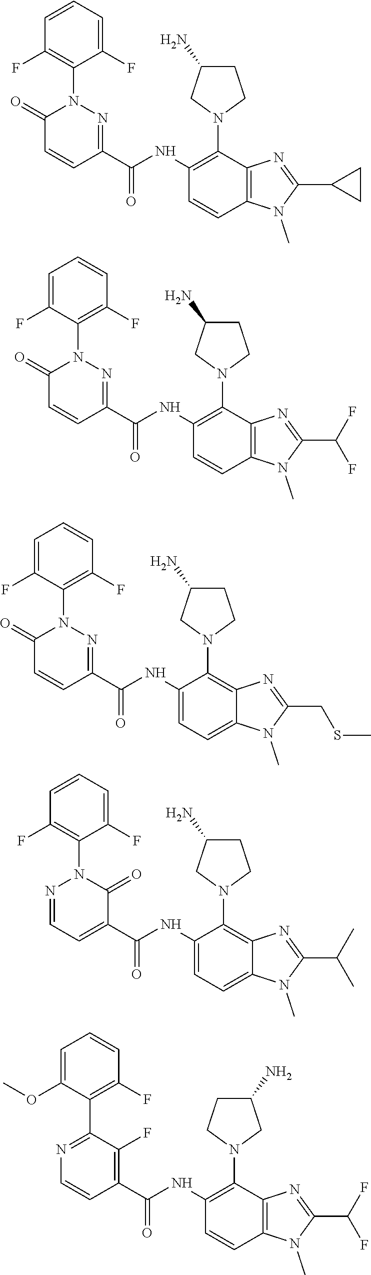

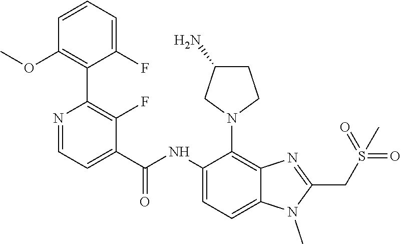 [SMILES: COc1cccc(F)c1-c1nccc(C(=O)Nc2ccc3c(nc(CS(C)(=O)=O)n3C)c2N2CC[C@@H](N)C2)c1F]

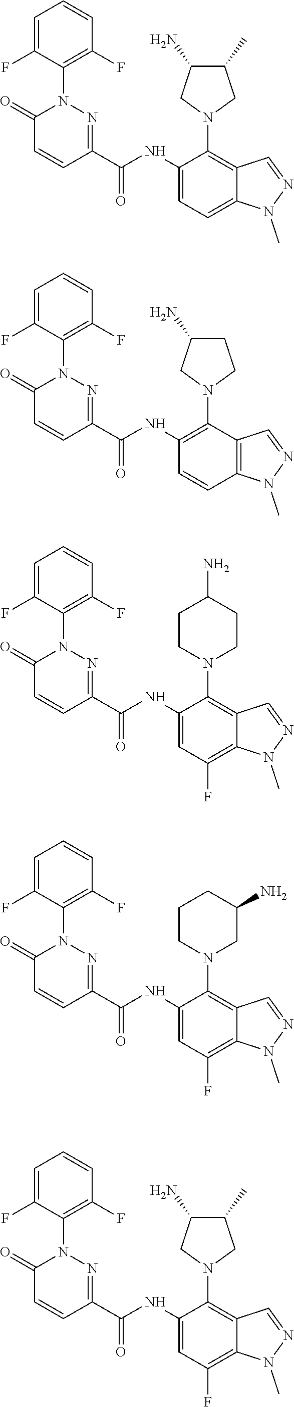 [SMILES: C[C@@H]1CN(c2c(NC(=O)c3ccc(=O)n(-c4c(F)cccc4F)n3)ccc3c2cnn3C)C[C@@H]1N ; Cn1ncc2c(N3CC[C@@H](N)C3)c(NC(=O)c3ccc(=O)n(-c4c(F)cccc4F)n3)ccc21 ; Cn1ncc2c(N3CCC(N)CC3)c(NC(=O)c3ccc(=O)n(-c4c(F)cccc4F)n3)cc(F)c21 ; Cn1ncc2c(N3CCC[C@@H](N)C3)c(NC(=O)c3ccc(=O)n(-c4c(F)cccc4F)n3)cc(F)c21 ; C[C@@H]1CN(c2c(NC(=O)c3ccc(=O)n(-c4c(F)cccc4F)n3)cc(F)c3c2cnn3C)C[C@@H]1N]

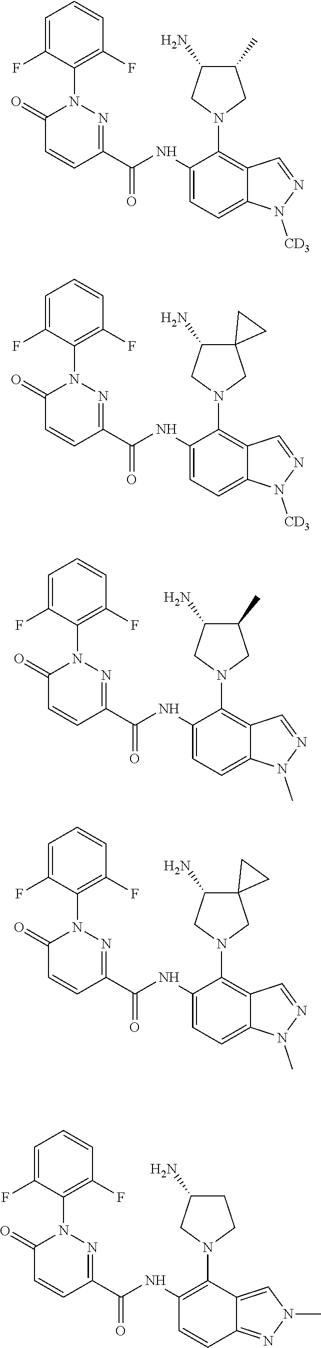 [SMILES: [2H]C([2H])([2H])n1ncc2c(N3C[C@@H](C)[C@@H](N)C3)c(NC(=O)c3ccc(=O)n(-c4c(F)cccc4F)n3)ccc21 ; [2H]C([2H])([2H])n1ncc2c(N3C[C@H](N)C4(CC4)C3)c(NC(=O)c3ccc(=O)n(-c4c(F)cccc4F)n3)ccc21 ; C[C@H]1CN(c2c(NC(=O)c3ccc(=O)n(-c4c(F)cccc4F)n3)ccc3c2cnn3C)C[C@@H]1N ; Cn1ncc2c(N3C[C@H](N)C4(CC4)C3)c(NC(=O)c3ccc(=O)n(-c4c(F)cccc4F)n3)ccc21 ; Cn1cc2c(N3CC[C@@H](N)C3)c(NC(=O)c3ccc(=O)n(-c4c(F)cccc4F)n3)ccc2n1]

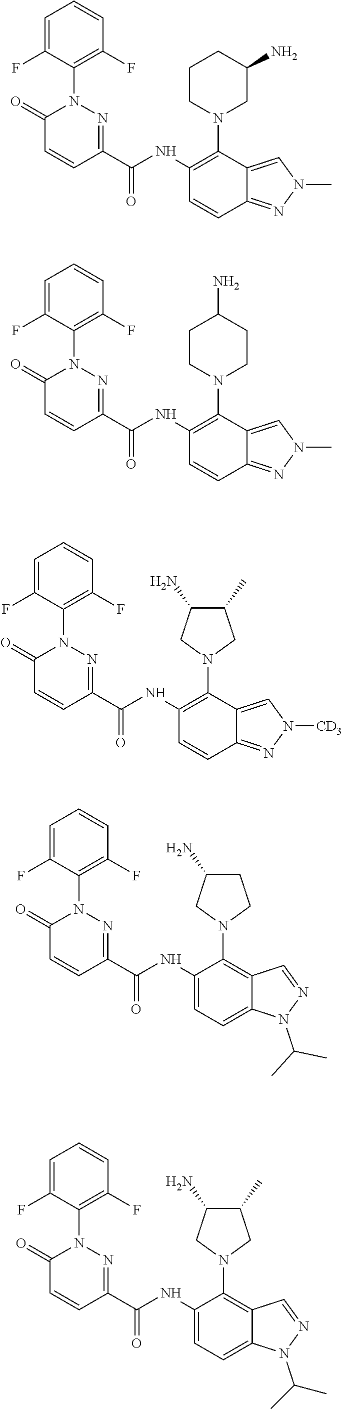 [SMILES: Cn1cc2c(N3CCC[C@@H](N)C3)c(NC(=O)c3ccc(=O)n(-c4c(F)cccc4F)n3)ccc2n1 ; Cn1cc2c(N3CCC(N)CC3)c(NC(=O)c3ccc(=O)n(-c4c(F)cccc4F)n3)ccc2n1 ; [2H]C([2H])([2H])n1cc2c(N3C[C@@H](C)[C@@H](N)C3)c(NC(=O)c3ccc(=O)n(-c4c(F)cccc4F)n3)ccc2n1 ; CC(C)n1ncc2c(N3CC[C@@H](N)C3)c(NC(=O)c3ccc(=O)n(-c4c(F)cccc4F)n3)ccc21 ; CC(C)n1ncc2c(N3C[C@@H](C)[C@@H](N)C3)c(NC(=O)c3ccc(=O)n(-c4c(F)cccc4F)n3)ccc21]

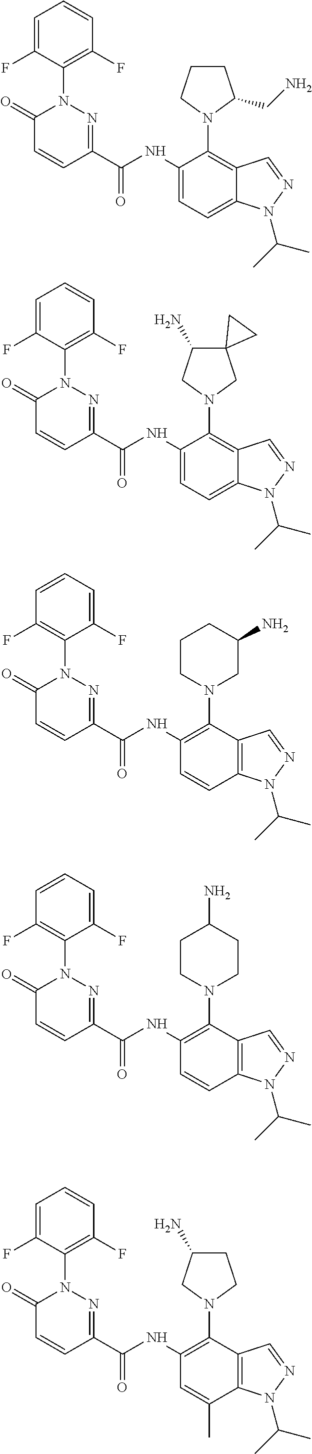 [SMILES: CC(C)n1ncc2c(N3CCC[C@@H]3CN)c(NC(=O)c3ccc(=O)n(-c4c(F)cccc4F)n3)ccc21 ; CC(C)n1ncc2c(N3C[C@H](N)C4(CC4)C3)c(NC(=O)c3ccc(=O)n(-c4c(F)cccc4F)n3)ccc21 ; CC(C)n1ncc2c(N3CCC[C@@H](N)C3)c(NC(=O)c3ccc(=O)n(-c4c(F)cccc4F)n3)ccc21 ; CC(C)n1ncc2c(N3CCC(N)CC3)c(NC(=O)c3ccc(=O)n(-c4c(F)cccc4F)n3)ccc21 ; Cc1cc(NC(=O)c2ccc(=O)n(-c3c(F)cccc3F)n2)c(N2CC[C@@H](N)C2)c2cnn(C(C)C)c12]

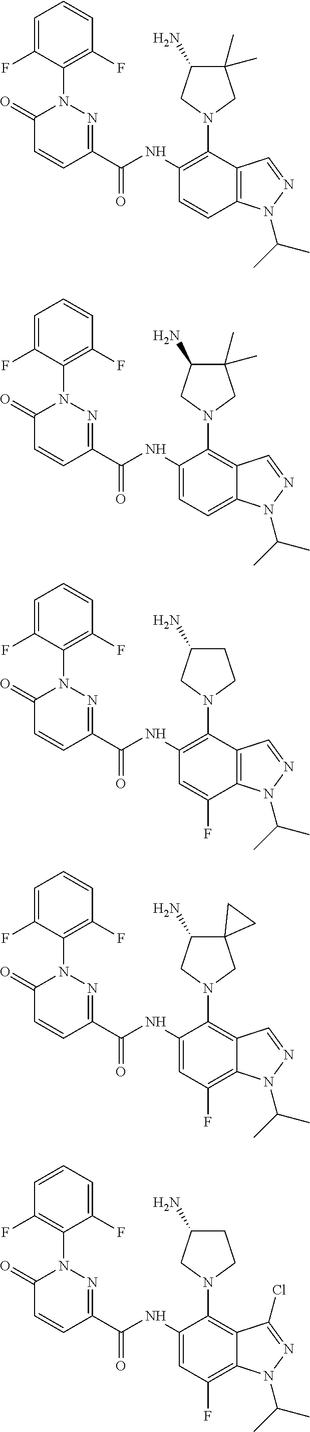 [SMILES: CC(C)n1ncc2c(N3C[C@H](N)C(C)(C)C3)c(NC(=O)c3ccc(=O)n(-c4c(F)cccc4F)n3)ccc21 ; CC(C)n1ncc2c(N3C[C@@H](N)C(C)(C)C3)c(NC(=O)c3ccc(=O)n(-c4c(F)cccc4F)n3)ccc21 ; CC(C)n1ncc2c(N3CC[C@@H](N)C3)c(NC(=O)c3ccc(=O)n(-c4c(F)cccc4F)n3)cc(F)c21 ; CC(C)n1ncc2c(N3C[C@H](N)C4(CC4)C3)c(NC(=O)c3ccc(=O)n(-c4c(F)cccc4F)n3)cc(F)c21 ; CC(C)n1nc(Cl)c2c(N3CC[C@@H](N)C3)c(NC(=O)c3ccc(=O)n(-c4c(F)cccc4F)n3)cc(F)c21]

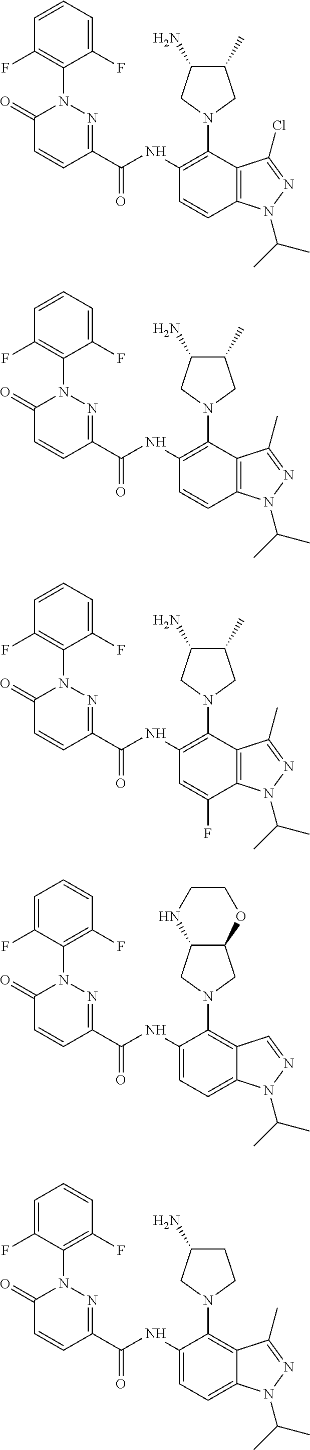 [SMILES: CC(C)n1nc(Cl)c2c(N3C[C@@H](C)[C@@H](N)C3)c(NC(=O)c3ccc(=O)n(-c4c(F)cccc4F)n3)ccc21 ; Cc1nn(C(C)C)c2ccc(NC(=O)c3ccc(=O)n(-c4c(F)cccc4F)n3)c(N3C[C@@H](C)[C@@H](N)C3)c12 ; Cc1nn(C(C)C)c2c(F)cc(NC(=O)c3ccc(=O)n(-c4c(F)cccc4F)n3)c(N3C[C@@H](C)[C@@H](N)C3)c12 ; CC(C)n1ncc2c(N3C[C@@H]4NCCO[C@H]4C3)c(NC(=O)c3ccc(=O)n(-c4c(F)cccc4F)n3)ccc21 ; Cc1nn(C(C)C)c2ccc(NC(=O)c3ccc(=O)n(-c4c(F)cccc4F)n3)c(N3CC[C@@H](N)C3)c12]

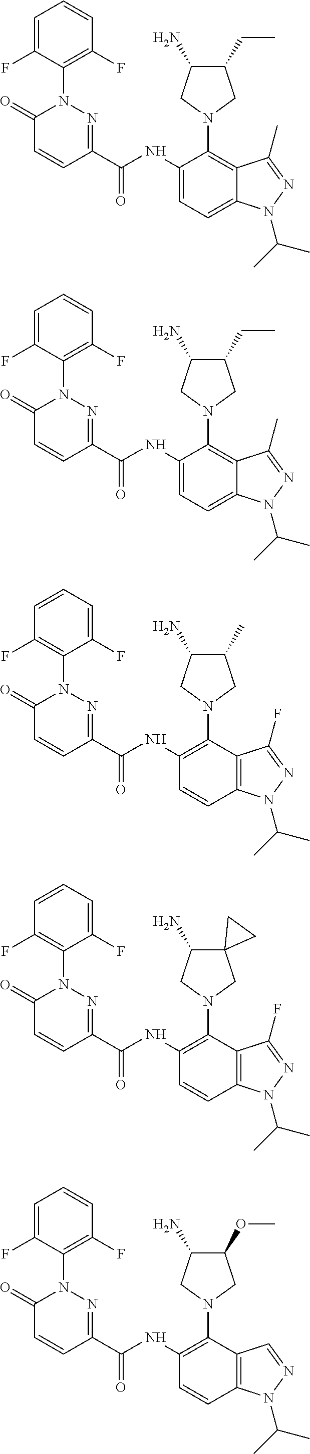 [SMILES: CC[C@@H]1CN(c2c(NC(=O)c3ccc(=O)n(-c4c(F)cccc4F)n3)ccc3c2c(C)nn3C(C)C)C[C@@H]1N ; CC[C@@H]1CN(c2c(NC(=O)c3ccc(=O)n(-c4c(F)cccc4F)n3)ccc3c2c(C)nn3C(C)C)C[C@@H]1N ; CC(C)n1nc(F)c2c(N3C[C@@H](C)[C@@H](N)C3)c(NC(=O)c3ccc(=O)n(-c4c(F)cccc4F)n3)ccc21 ; CC(C)n1nc(F)c2c(N3C[C@H](N)C4(CC4)C3)c(NC(=O)c3ccc(=O)n(-c4c(F)cccc4F)n3)ccc21 ; CO[C@H]1CN(c2c(NC(=O)c3ccc(=O)n(-c4c(F)cccc4F)n3)ccc3c2cnn3C(C)C)C[C@@H]1N]

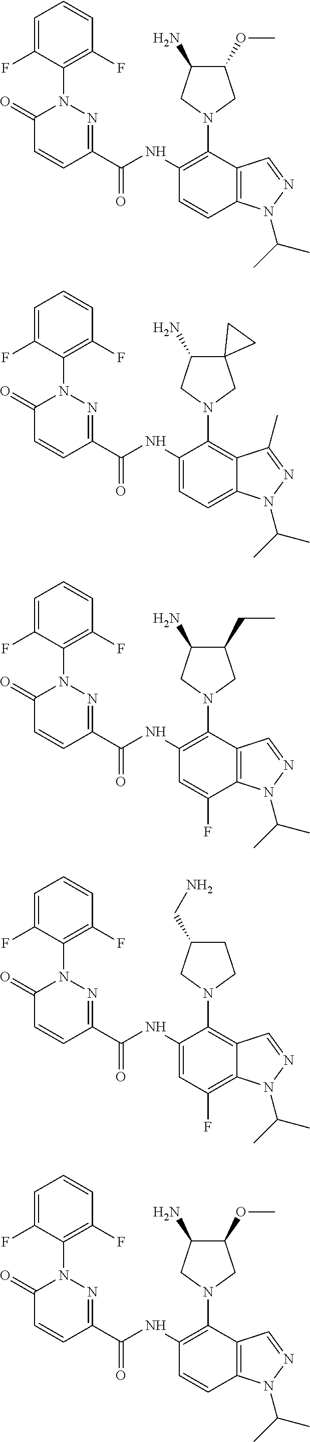 [SMILES: CO[C@@H]1CN(c2c(NC(=O)c3ccc(=O)n(-c4c(F)cccc4F)n3)ccc3c2cnn3C(C)C)C[C@H]1N ; Cc1nn(C(C)C)c2ccc(NC(=O)c3ccc(=O)n(-c4c(F)cccc4F)n3)c(N3C[C@H](N)C4(CC4)C3)c12 ; CC[C@H]1CN(c2c(NC(=O)c3ccc(=O)n(-c4c(F)cccc4F)n3)cc(F)c3c2cnn3C(C)C)C[C@H]1N ; CC(C)n1ncc2c(N3CC[C@@H](CN)C3)c(NC(=O)c3ccc(=O)n(-c4c(F)cccc4F)n3)cc(F)c21 ; CO[C@H]1CN(c2c(NC(=O)c3ccc(=O)n(-c4c(F)cccc4F)n3)ccc3c2cnn3C(C)C)C[C@H]1N]

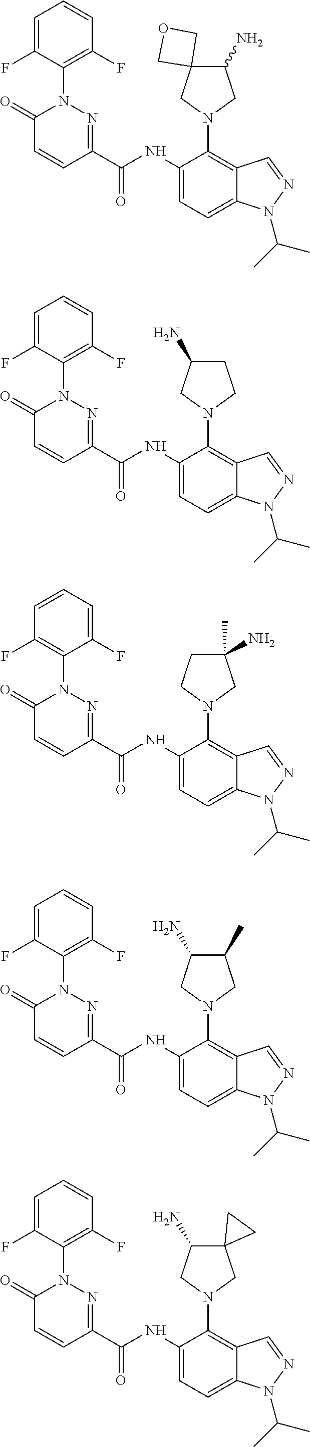 [SMILES: CC(C)n1ncc2c(N3CC(N)C4(COC4)C3)c(NC(=O)c3ccc(=O)n(-c4c(F)cccc4F)n3)ccc21 ; CC(C)n1ncc2c(N3CC[C@H](N)C3)c(NC(=O)c3ccc(=O)n(-c4c(F)cccc4F)n3)ccc21 ; CC(C)n1ncc2c(N3CC[C@@](C)(N)C3)c(NC(=O)c3ccc(=O)n(-c4c(F)cccc4F)n3)ccc21 ; CC(C)n1ncc2c(N3C[C@H](C)[C@@H](N)C3)c(NC(=O)c3ccc(=O)n(-c4c(F)cccc4F)n3)ccc21 ; CC(C)n1ncc2c(N3C[C@H](N)C4(CC4)C3)c(NC(=O)c3ccc(=O)n(-c4c(F)cccc4F)n3)ccc21]

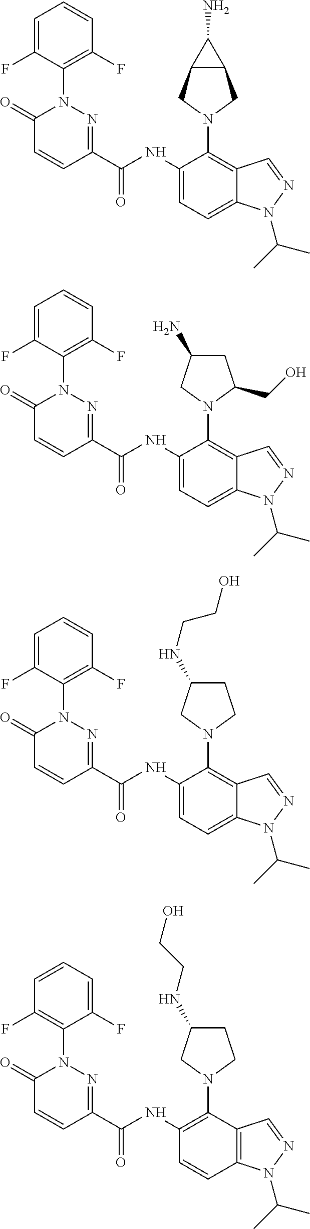

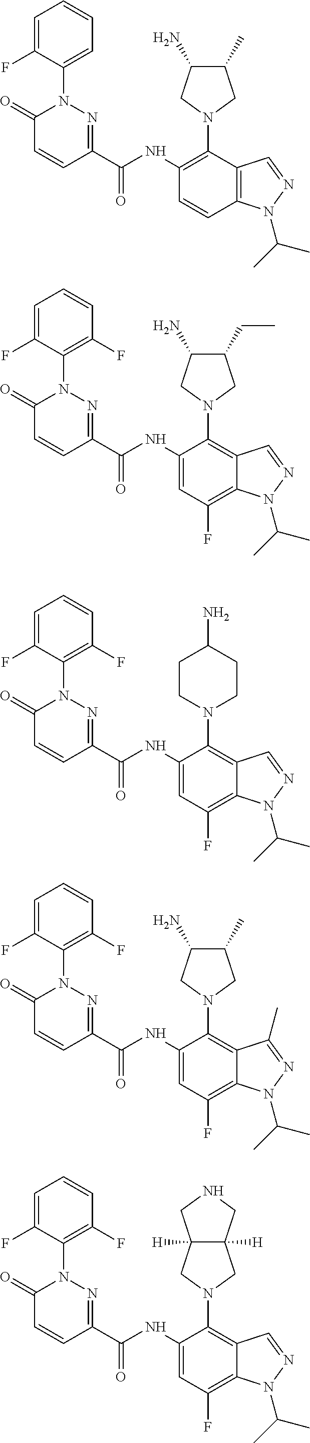 [SMILES: CC(C)n1ncc2c(N3C[C@@H](C)[C@@H](N)C3)c(NC(=O)c3ccc(=O)n(-c4ccccc4F)n3)ccc21 ; CC[C@@H]1CN(c2c(NC(=O)c3ccc(=O)n(-c4c(F)cccc4F)n3)cc(F)c3c2cnn3C(C)C)C[C@@H]1N ; CC(C)n1ncc2c(N3CCC(N)CC3)c(NC(=O)c3ccc(=O)n(-c4c(F)cccc4F)n3)cc(F)c21 ; Cc1nn(C(C)C)c2c(F)cc(NC(=O)c3ccc(=O)n(-c4c(F)cccc4F)n3)c(N3C[C@@H](C)[C@@H](N)C3)c12 ; CC(C)n1ncc2c(N3C[C@H]4CNC[C@H]4C3)c(NC(=O)c3ccc(=O)n(-c4c(F)cccc4F)n3)cc(F)c21]

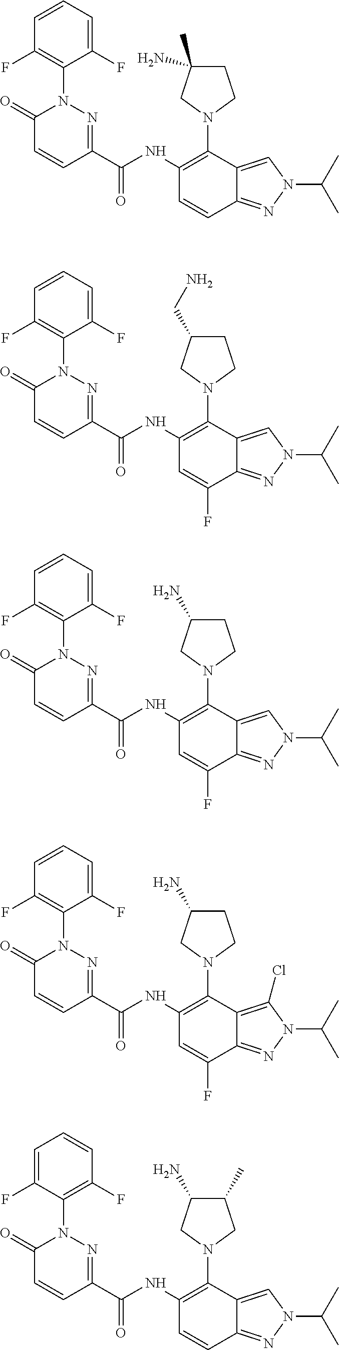 [SMILES: CC(C)n1cc2c(N3CC[C@@](C)(N)C3)c(NC(=O)c3ccc(=O)n(-c4c(F)cccc4F)n3)ccc2n1 ; CC(C)n1cc2c(N3CC[C@@H](CN)C3)c(NC(=O)c3ccc(=O)n(-c4c(F)cccc4F)n3)cc(F)c2n1 ; CC(C)n1cc2c(N3CC[C@@H](N)C3)c(NC(=O)c3ccc(=O)n(-c4c(F)cccc4F)n3)cc(F)c2n1 ; CC(C)n1nc2c(F)cc(NC(=O)c3ccc(=O)n(-c4c(F)cccc4F)n3)c(N3CC[C@@H](N)C3)c2c1Cl ; CC(C)n1cc2c(N3C[C@@H](C)[C@@H](N)C3)c(NC(=O)c3ccc(=O)n(-c4c(F)cccc4F)n3)ccc2n1]

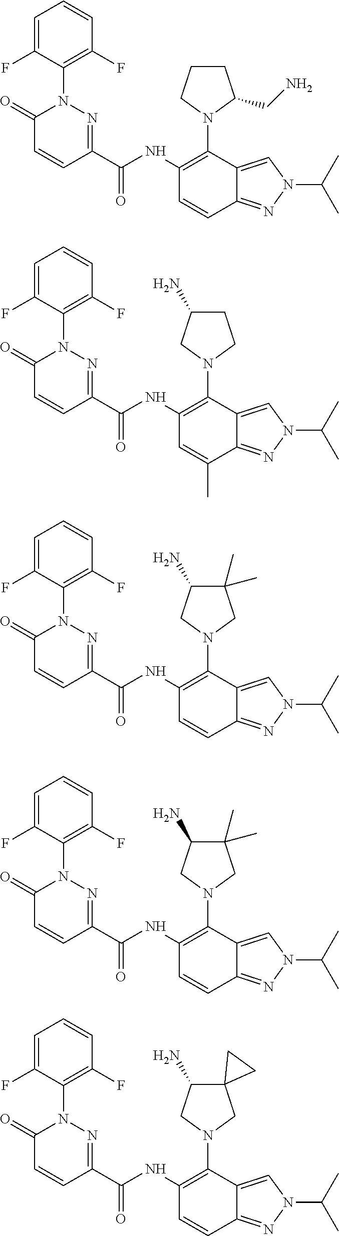 [SMILES: CC(C)n1cc2c(N3CCC[C@@H]3CN)c(NC(=O)c3ccc(=O)n(-c4c(F)cccc4F)n3)ccc2n1 ; Cc1cc(NC(=O)c2ccc(=O)n(-c3c(F)cccc3F)n2)c(N2CC[C@@H](N)C2)c2cn(C(C)C)nc12 ; CC(C)n1cc2c(N3C[C@H](N)C(C)(C)C3)c(NC(=O)c3ccc(=O)n(-c4c(F)cccc4F)n3)ccc2n1 ; CC(C)n1cc2c(N3C[C@@H](N)C(C)(C)C3)c(NC(=O)c3ccc(=O)n(-c4c(F)cccc4F)n3)ccc2n1 ; CC(C)n1cc2c(N3C[C@H](N)C4(CC4)C3)c(NC(=O)c3ccc(=O)n(-c4c(F)cccc4F)n3)ccc2n1]

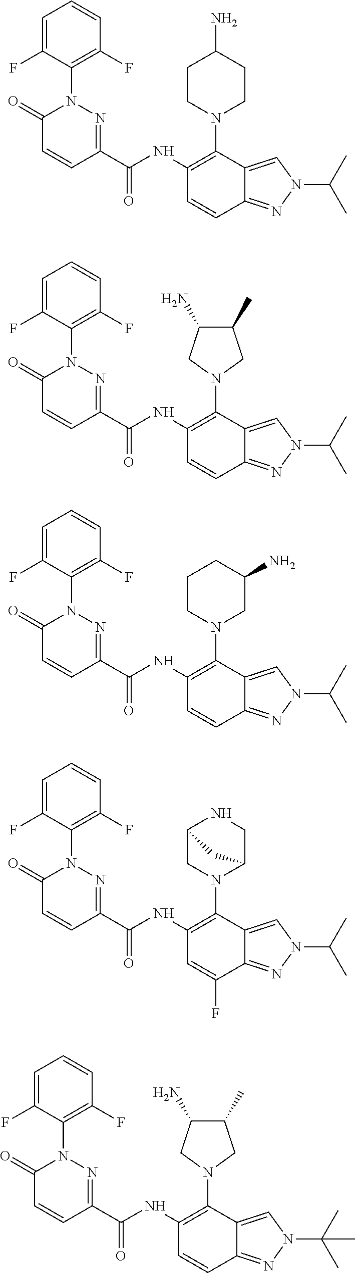 [SMILES: CC(C)n1cc2c(N3CCC(N)CC3)c(NC(=O)c3ccc(=O)n(-c4c(F)cccc4F)n3)ccc2n1 ; CC(C)n1cc2c(N3C[C@H](C)[C@@H](N)C3)c(NC(=O)c3ccc(=O)n(-c4c(F)cccc4F)n3)ccc2n1 ; CC(C)n1cc2c(N3CCC[C@@H](N)C3)c(NC(=O)c3ccc(=O)n(-c4c(F)cccc4F)n3)ccc2n1 ; CC(C)n1cc2c(N3C[C@@H]4C[C@H]3CN4)c(NC(=O)c3ccc(=O)n(-c4c(F)cccc4F)n3)cc(F)c2n1 ; C[C@@H]1CN(c2c(NC(=O)c3ccc(=O)n(-c4c(F)cccc4F)n3)ccc3nn(C(C)(C)C)cc23)C[C@@H]1N]

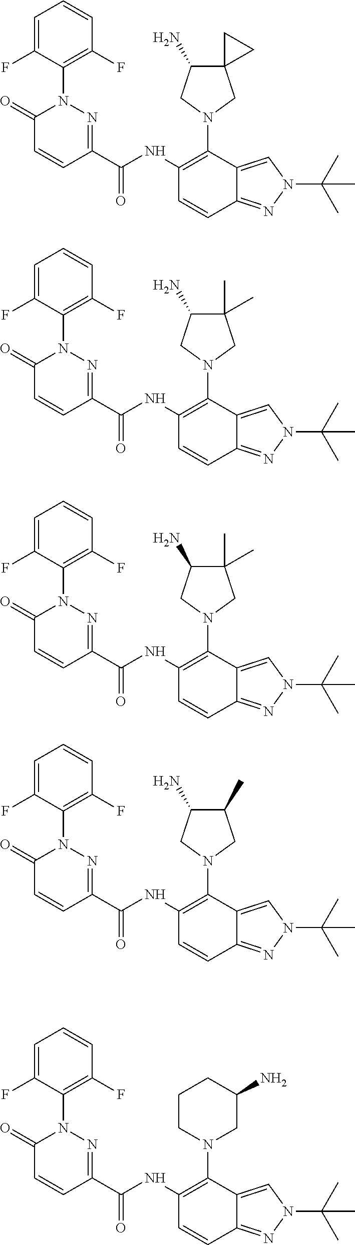 [SMILES: CC(C)(C)n1cc2c(N3C[C@H](N)C4(CC4)C3)c(NC(=O)c3ccc(=O)n(-c4c(F)cccc4F)n3)ccc2n1 ; CC1(C)CN(c2c(NC(=O)c3ccc(=O)n(-c4c(F)cccc4F)n3)ccc3nn(C(C)(C)C)cc23)C[C@@H]1N ; CC1(C)CN(c2c(NC(=O)c3ccc(=O)n(-c4c(F)cccc4F)n3)ccc3nn(C(C)(C)C)cc23)C[C@H]1N ; C[C@H]1CN(c2c(NC(=O)c3ccc(=O)n(-c4c(F)cccc4F)n3)ccc3nn(C(C)(C)C)cc23)C[C@@H]1N ; CC(C)(C)n1cc2c(N3CCC[C@@H](N)C3)c(NC(=O)c3ccc(=O)n(-c4c(F)cccc4F)n3)ccc2n1]

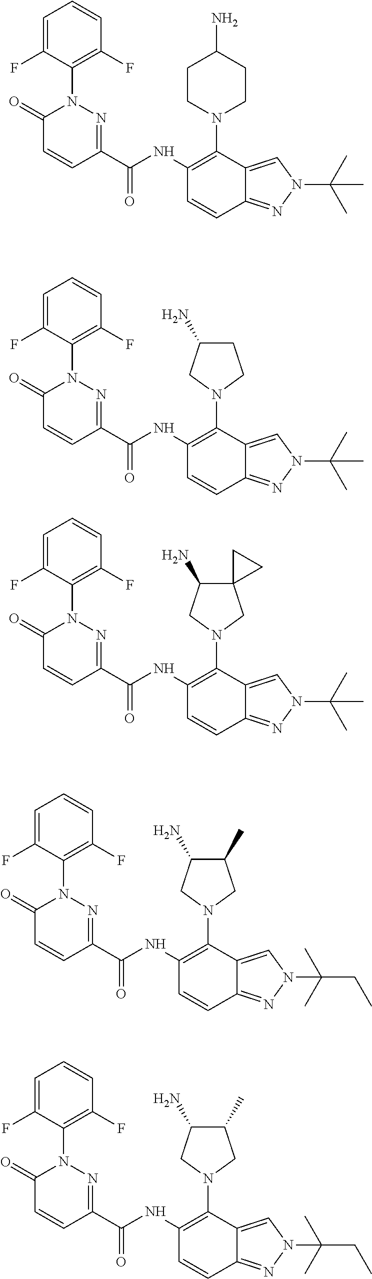 [SMILES: CC(C)(C)n1cc2c(N3CCC(N)CC3)c(NC(=O)c3ccc(=O)n(-c4c(F)cccc4F)n3)ccc2n1 ; CC(C)(C)n1cc2c(N3CC[C@@H](N)C3)c(NC(=O)c3ccc(=O)n(-c4c(F)cccc4F)n3)ccc2n1 ; CC(C)(C)n1cc2c(N3C[C@@H](N)C4(CC4)C3)c(NC(=O)c3ccc(=O)n(-c4c(F)cccc4F)n3)ccc2n1 ; CCC(C)(C)n1cc2c(N3C[C@H](C)[C@@H](N)C3)c(NC(=O)c3ccc(=O)n(-c4c(F)cccc4F)n3)ccc2n1 ; CCC(C)(C)n1cc2c(N3C[C@@H](C)[C@@H](N)C3)c(NC(=O)c3ccc(=O)n(-c4c(F)cccc4F)n3)ccc2n1]

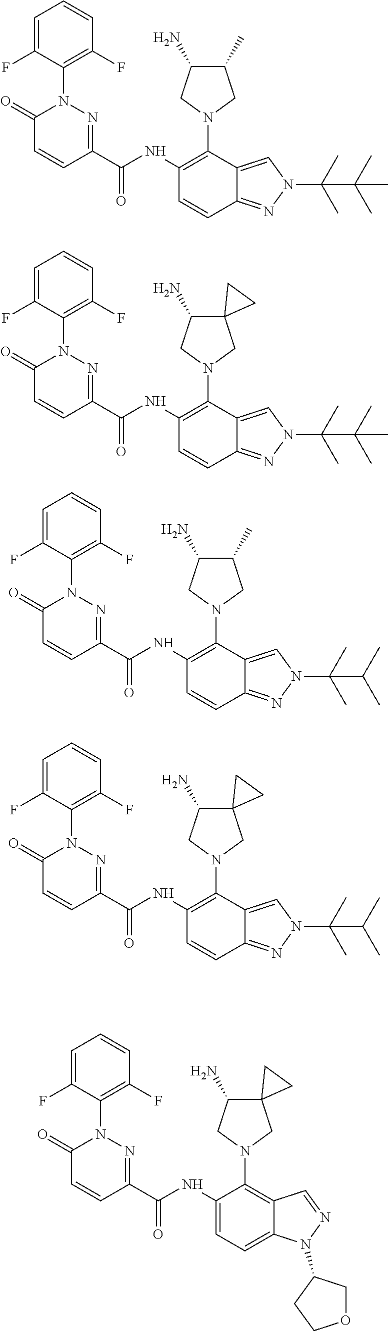 [SMILES: C[C@@H]1CN(c2c(NC(=O)c3ccc(=O)n(-c4c(F)cccc4F)n3)ccc3nn(C(C)(C)C(C)(C)C)cc23)C[C@@H]1N ; CC(C)(C)C(C)(C)n1cc2c(N3C[C@H](N)C4(CC4)C3)c(NC(=O)c3ccc(=O)n(-c4c(F)cccc4F)n3)ccc2n1 ; CC(C)C(C)(C)n1cc2c(N3C[C@@H](C)[C@@H](N)C3)c(NC(=O)c3ccc(=O)n(-c4c(F)cccc4F)n3)ccc2n1 ; CC(C)C(C)(C)n1cc2c(N3C[C@H](N)C4(CC4)C3)c(NC(=O)c3ccc(=O)n(-c4c(F)cccc4F)n3)ccc2n1 ; N[C@H]1CN(c2c(NC(=O)c3ccc(=O)n(-c4c(F)cccc4F)n3)ccc3c2cnn3[C@H]2CCOC2)CC12CC2]

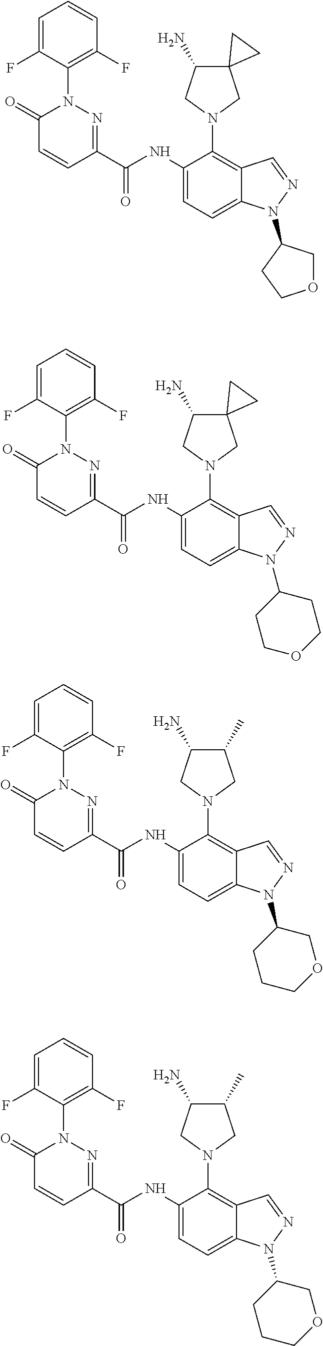 [SMILES: N[C@H]1CN(c2c(NC(=O)c3ccc(=O)n(-c4c(F)cccc4F)n3)ccc3c2cnn3[C@@H]2CCOC2)CC12CC2 ; N[C@H]1CN(c2c(NC(=O)c3ccc(=O)n(-c4c(F)cccc4F)n3)ccc3c2cnn3C2CCOCC2)CC12CC2 ; C[C@@H]1CN(c2c(NC(=O)c3ccc(=O)n(-c4c(F)cccc4F)n3)ccc3c2cnn3[C@@H]2CCCOC2)C[C@@H]1N ; C[C@@H]1CN(c2c(NC(=O)c3ccc(=O)n(-c4c(F)cccc4F)n3)ccc3c2cnn3[C@H]2CCCOC2)C[C@@H]1N]

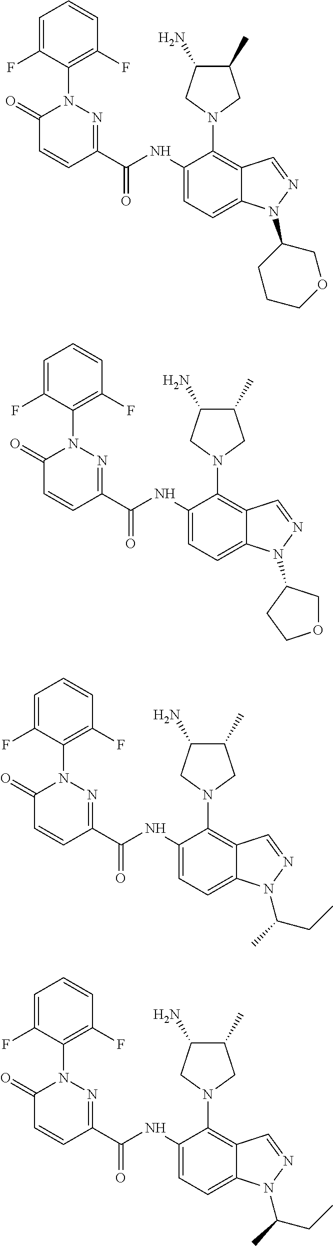 [SMILES: C[C@H]1CN(c2c(NC(=O)c3ccc(=O)n(-c4c(F)cccc4F)n3)ccc3c2cnn3[C@@H]2CCCOC2)C[C@@H]1N ; C[C@@H]1CN(c2c(NC(=O)c3ccc(=O)n(-c4c(F)cccc4F)n3)ccc3c2cnn3[C@H]2CCOC2)C[C@@H]1N ; CC[C@H](C)n1ncc2c(N3C[C@@H](C)[C@@H](N)C3)c(NC(=O)c3ccc(=O)n(-c4c(F)cccc4F)n3)ccc21 ; CC[C@@H](C)n1ncc2c(N3C[C@@H](C)[C@@H](N)C3)c(NC(=O)c3ccc(=O)n(-c4c(F)cccc4F)n3)ccc21]

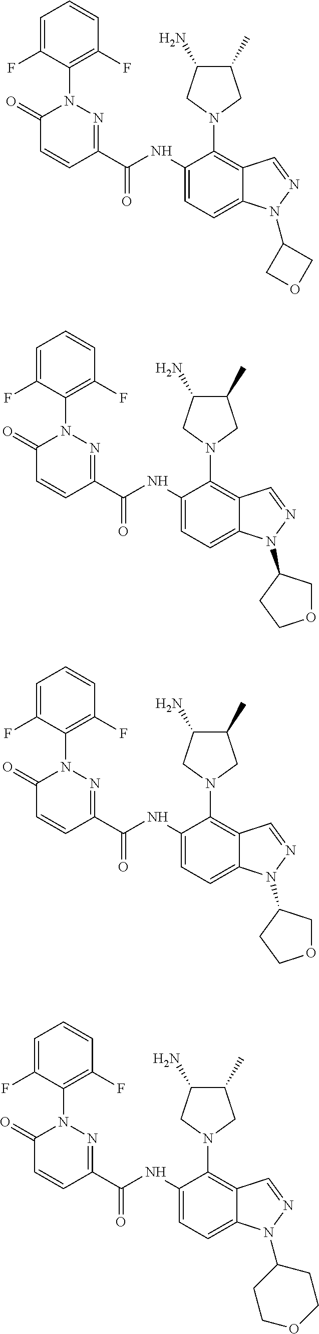 [SMILES: C[C@@H]1CN(c2c(NC(=O)c3ccc(=O)n(-c4c(F)cccc4F)n3)ccc3c2cnn3C2COC2)C[C@@H]1N ; C[C@H]1CN(c2c(NC(=O)c3ccc(=O)n(-c4c(F)cccc4F)n3)ccc3c2cnn3[C@@H]2CCOC2)C[C@@H]1N ; C[C@H]1CN(c2c(NC(=O)c3ccc(=O)n(-c4c(F)cccc4F)n3)ccc3c2cnn3[C@H]2CCOC2)C[C@@H]1N ; C[C@@H]1CN(c2c(NC(=O)c3ccc(=O)n(-c4c(F)cccc4F)n3)ccc3c2cnn3C2CCOCC2)C[C@@H]1N]

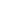

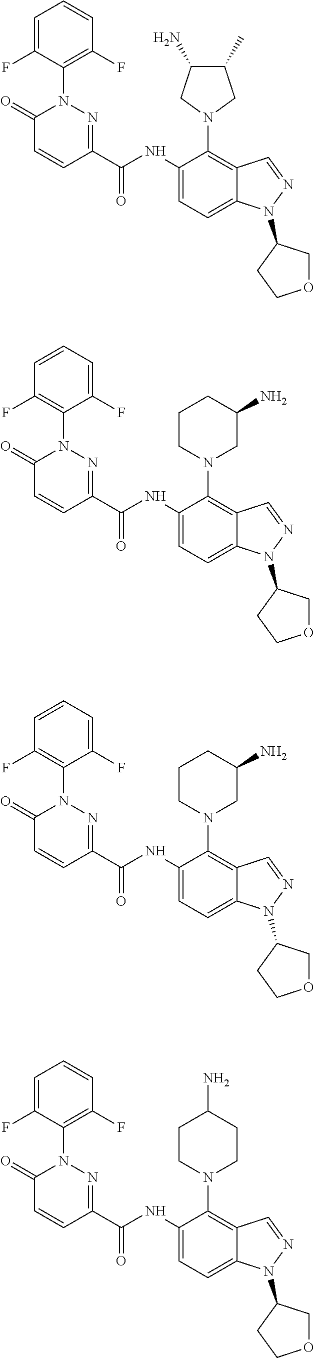 [SMILES: C[C@@H]1CN(c2c(NC(=O)c3ccc(=O)n(-c4c(F)cccc4F)n3)ccc3c2cnn3[C@@H]2CCOC2)C[C@@H]1N ; N[C@@H]1CCCN(c2c(NC(=O)c3ccc(=O)n(-c4c(F)cccc4F)n3)ccc3c2cnn3[C@@H]2CCOC2)C1 ; N[C@@H]1CCCN(c2c(NC(=O)c3ccc(=O)n(-c4c(F)cccc4F)n3)ccc3c2cnn3[C@H]2CCOC2)C1 ; NC1CCN(c2c(NC(=O)c3ccc(=O)n(-c4c(F)cccc4F)n3)ccc3c2cnn3[C@@H]2CCOC2)CC1]

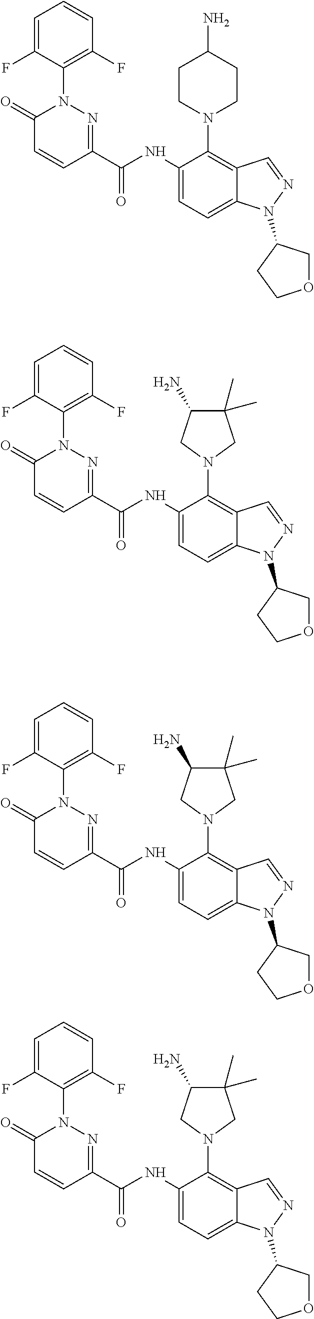 [SMILES: NC1CCN(c2c(NC(=O)c3ccc(=O)n(-c4c(F)cccc4F)n3)ccc3c2cnn3[C@H]2CCOC2)CC1 ; CC1(C)CN(c2c(NC(=O)c3ccc(=O)n(-c4c(F)cccc4F)n3)ccc3c2cnn3[C@@H]2CCOC2)C[C@@H]1N ; CC1(C)CN(c2c(NC(=O)c3ccc(=O)n(-c4c(F)cccc4F)n3)ccc3c2cnn3[C@@H]2CCOC2)C[C@H]1N ; CC1(C)CN(c2c(NC(=O)c3ccc(=O)n(-c4c(F)cccc4F)n3)ccc3c2cnn3[C@H]2CCOC2)C[C@@H]1N]

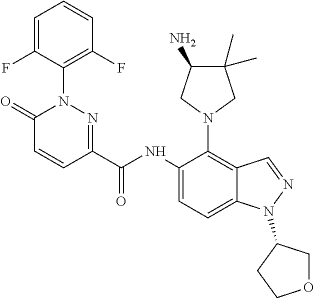 [SMILES: CC1(C)CN(c2c(NC(=O)c3ccc(=O)n(-c4c(F)cccc4F)n3)ccc3c2cnn3[C@H]2CCOC2)C[C@H]1N]

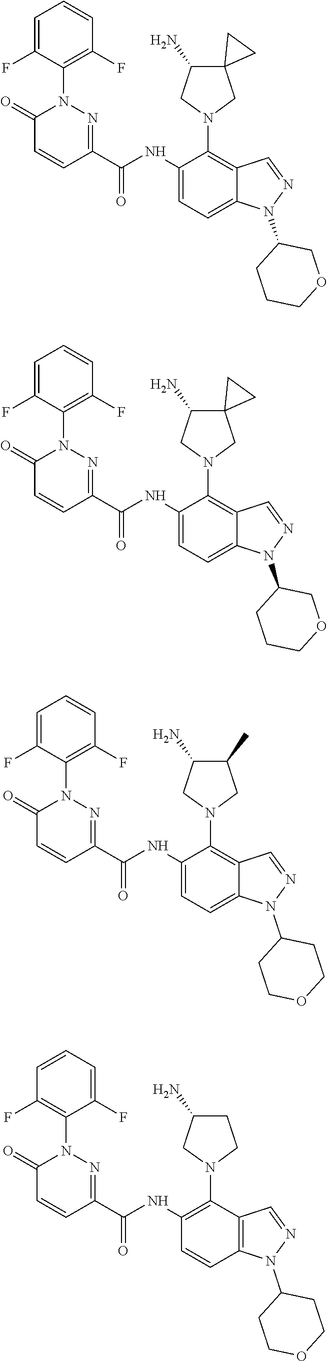 [SMILES: N[C@H]1CN(c2c(NC(=O)c3ccc(=O)n(-c4c(F)cccc4F)n3)ccc3c2cnn3[C@H]2CCCOC2)CC12CC2 ; N[C@H]1CN(c2c(NC(=O)c3ccc(=O)n(-c4c(F)cccc4F)n3)ccc3c2cnn3[C@@H]2CCCOC2)CC12CC2 ; C[C@H]1CN(c2c(NC(=O)c3ccc(=O)n(-c4c(F)cccc4F)n3)ccc3c2cnn3C2CCOCC2)C[C@@H]1N ; N[C@@H]1CCN(c2c(NC(=O)c3ccc(=O)n(-c4c(F)cccc4F)n3)ccc3c2cnn3C2CCOCC2)C1]

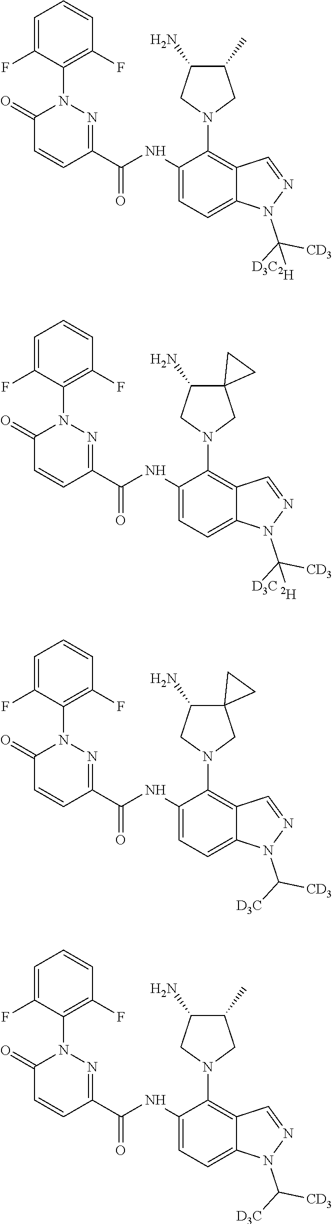 [SMILES: [2H]C([2H])([2H])C([2H])(n1ncc2c(N3C[C@@H](C)[C@@H](N)C3)c(NC(=O)c3ccc(=O)n(-c4c(F)cccc4F)n3)ccc21)C([2H])([2H])[2H] ; [2H]C([2H])([2H])C([2H])(n1ncc2c(N3C[C@H](N)C4(CC4)C3)c(NC(=O)c3ccc(=O)n(-c4c(F)cccc4F)n3)ccc21)C([2H])([2H])[2H] ; [2H]C([2H])([2H])C(n1ncc2c(N3C[C@H](N)C4(CC4)C3)c(NC(=O)c3ccc(=O)n(-c4c(F)cccc4F)n3)ccc21)C([2H])([2H])[2H] ; [2H]C([2H])([2H])C(n1ncc2c(N3C[C@@H](C)[C@@H](N)C3)c(NC(=O)c3ccc(=O)n(-c4c(F)cccc4F)n3)ccc21)C([2H])([2H])[2H]]

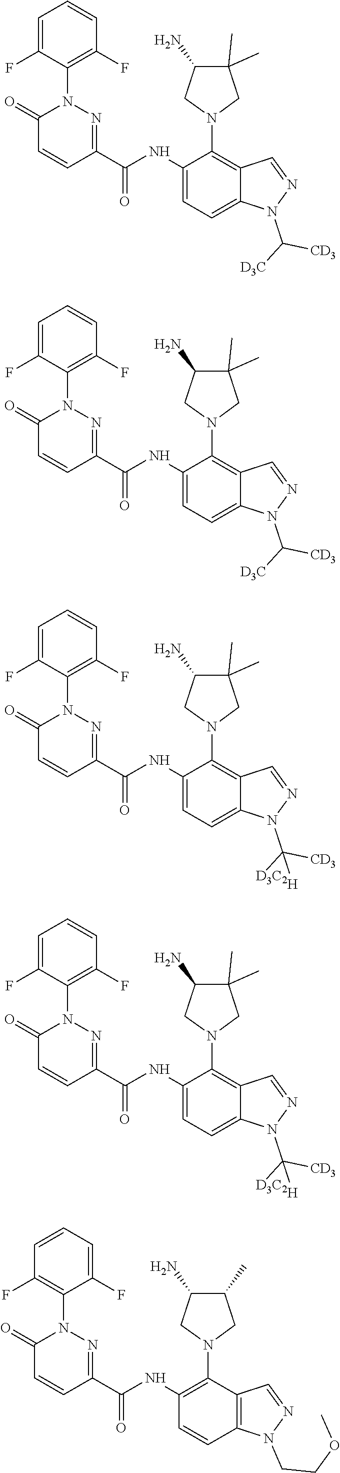

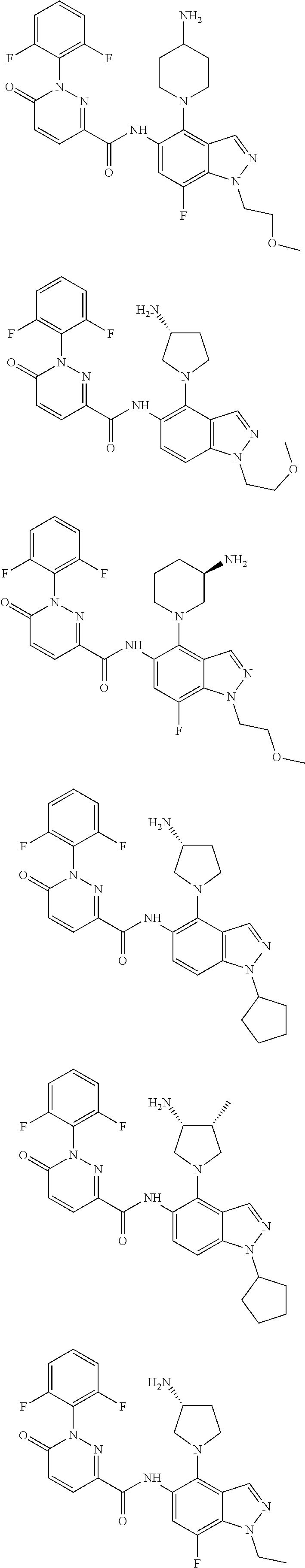 [SMILES: COCCn1ncc2c(N3CCC(N)CC3)c(NC(=O)c3ccc(=O)n(-c4c(F)cccc4F)n3)cc(F)c21 ; COCCn1ncc2c(N3CC[C@@H](N)C3)c(NC(=O)c3ccc(=O)n(-c4c(F)cccc4F)n3)ccc21 ; COCCn1ncc2c(N3CCC[C@@H](N)C3)c(NC(=O)c3ccc(=O)n(-c4c(F)cccc4F)n3)cc(F)c21 ; N[C@@H]1CCN(c2c(NC(=O)c3ccc(=O)n(-c4c(F)cccc4F)n3)ccc3c2cnn3C2CCCC2)C1 ; C[C@@H]1CN(c2c(NC(=O)c3ccc(=O)n(-c4c(F)cccc4F)n3)ccc3c2cnn3C2CCCC2)C[C@@H]1N ; CCn1ncc2c(N3CC[C@@H](N)C3)c(NC(=O)c3ccc(=O)n(-c4c(F)cccc4F)n3)cc(F)c21]

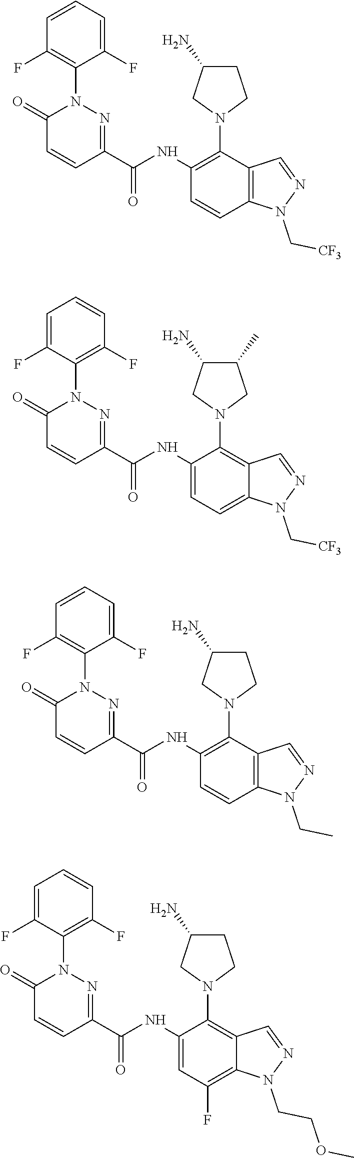 [SMILES: N[C@@H]1CCN(c2c(NC(=O)c3ccc(=O)n(-c4c(F)cccc4F)n3)ccc3c2cnn3CC(F)(F)F)C1 ; C[C@@H]1CN(c2c(NC(=O)c3ccc(=O)n(-c4c(F)cccc4F)n3)ccc3c2cnn3CC(F)(F)F)C[C@@H]1N ; CCn1ncc2c(N3CC[C@@H](N)C3)c(NC(=O)c3ccc(=O)n(-c4c(F)cccc4F)n3)ccc21 ; COCCn1ncc2c(N3CC[C@@H](N)C3)c(NC(=O)c3ccc(=O)n(-c4c(F)cccc4F)n3)cc(F)c21]

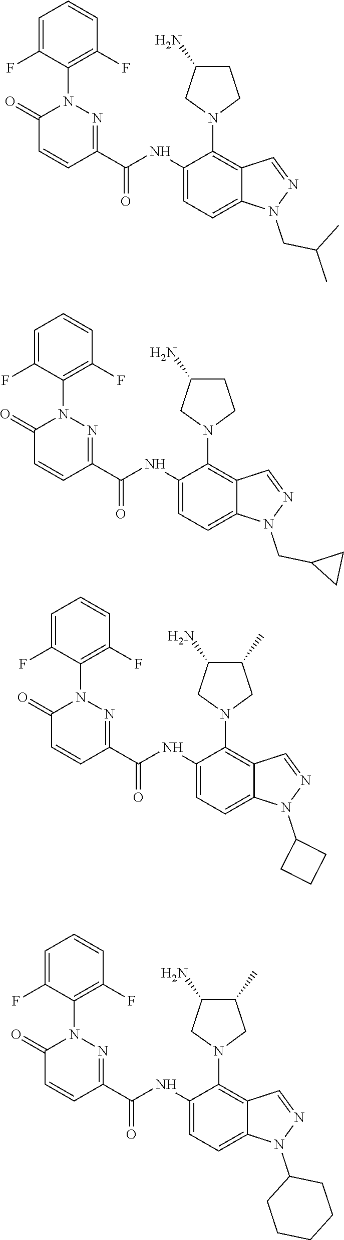 [SMILES: CC(C)Cn1ncc2c(N3CC[C@@H](N)C3)c(NC(=O)c3ccc(=O)n(-c4c(F)cccc4F)n3)ccc21 ; N[C@@H]1CCN(c2c(NC(=O)c3ccc(=O)n(-c4c(F)cccc4F)n3)ccc3c2cnn3CC2CC2)C1 ; C[C@@H]1CN(c2c(NC(=O)c3ccc(=O)n(-c4c(F)cccc4F)n3)ccc3c2cnn3C2CCC2)C[C@@H]1N ; C[C@@H]1CN(c2c(NC(=O)c3ccc(=O)n(-c4c(F)cccc4F)n3)ccc3c2cnn3C2CCCCC2)C[C@@H]1N]

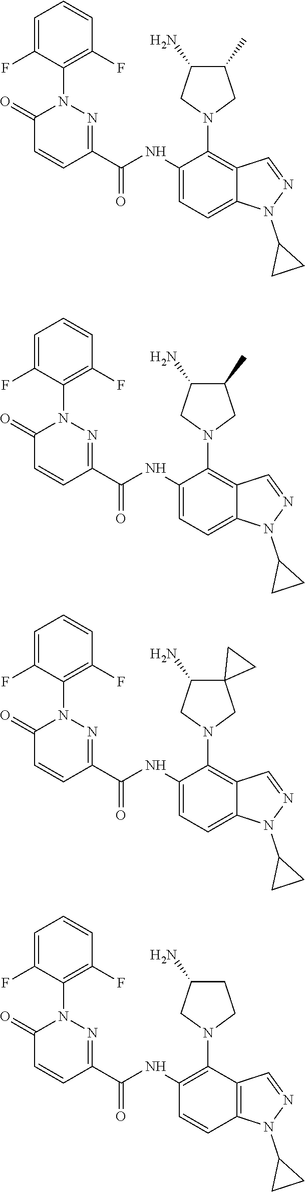 [SMILES: C[C@@H]1CN(c2c(NC(=O)c3ccc(=O)n(-c4c(F)cccc4F)n3)ccc3c2cnn3C2CC2)C[C@@H]1N ; C[C@H]1CN(c2c(NC(=O)c3ccc(=O)n(-c4c(F)cccc4F)n3)ccc3c2cnn3C2CC2)C[C@@H]1N ; N[C@H]1CN(c2c(NC(=O)c3ccc(=O)n(-c4c(F)cccc4F)n3)ccc3c2cnn3C2CC2)CC12CC2 ; N[C@@H]1CCN(c2c(NC(=O)c3ccc(=O)n(-c4c(F)cccc4F)n3)ccc3c2cnn3C2CC2)C1]

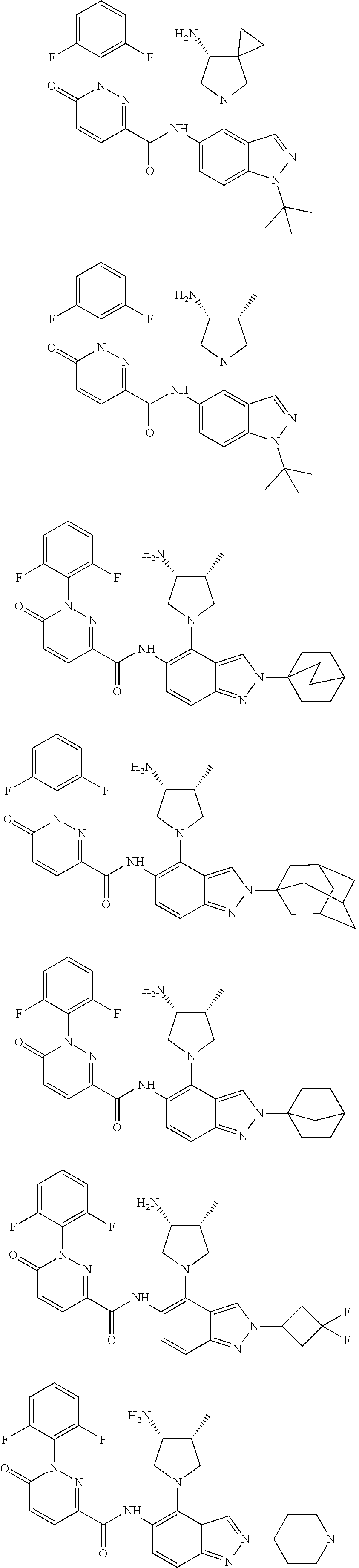

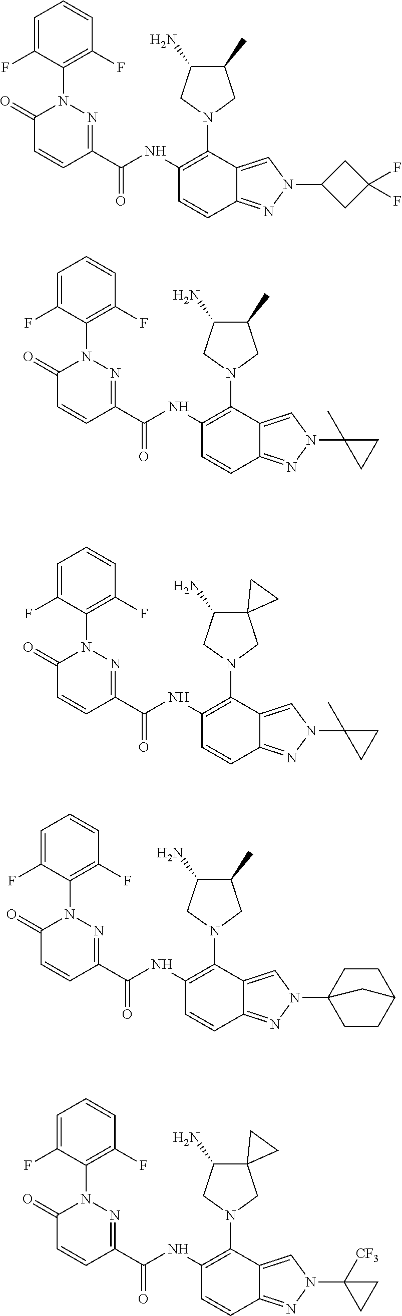 [SMILES: C[C@H]1CN(c2c(NC(=O)c3ccc(=O)n(-c4c(F)cccc4F)n3)ccc3nn(C4CC(F)(F)C4)cc23)C[C@@H]1N ; C[C@H]1CN(c2c(NC(=O)c3ccc(=O)n(-c4c(F)cccc4F)n3)ccc3nn(C4(C)CC4)cc23)C[C@@H]1N ; CC1(n2cc3c(N4C[C@H](N)C5(CC5)C4)c(NC(=O)c4ccc(=O)n(-c5c(F)cccc5F)n4)ccc3n2)CC1 ; C[C@H]1CN(c2c(NC(=O)c3ccc(=O)n(-c4c(F)cccc4F)n3)ccc3nn(C45CCC(CC4)C5)cc23)C[C@@H]1N ; N[C@H]1CN(c2c(NC(=O)c3ccc(=O)n(-c4c(F)cccc4F)n3)ccc3nn(C4(C(F)(F)F)CC4)cc23)CC12CC2]

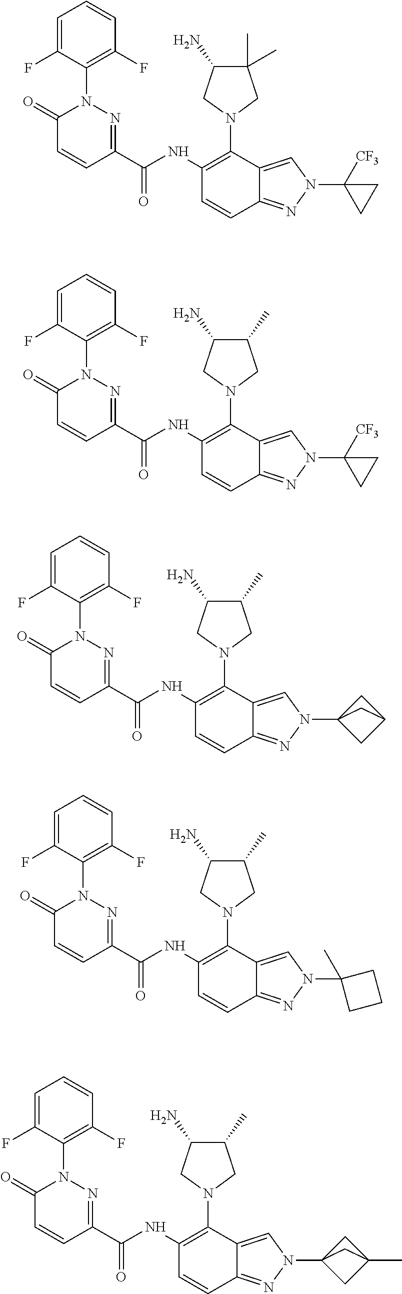 [SMILES: CC1(C)CN(c2c(NC(=O)c3ccc(=O)n(-c4c(F)cccc4F)n3)ccc3nn(C4(C(F)(F)F)CC4)cc23)C[C@@H]1N ; C[C@@H]1CN(c2c(NC(=O)c3ccc(=O)n(-c4c(F)cccc4F)n3)ccc3nn(C4(C(F)(F)F)CC4)cc23)C[C@@H]1N ; C[C@@H]1CN(c2c(NC(=O)c3ccc(=O)n(-c4c(F)cccc4F)n3)ccc3nn(C45CC(C4)C5)cc23)C[C@@H]1N ; C[C@@H]1CN(c2c(NC(=O)c3ccc(=O)n(-c4c(F)cccc4F)n3)ccc3nn(C4(C)CCC4)cc23)C[C@@H]1N ; C[C@@H]1CN(c2c(NC(=O)c3ccc(=O)n(-c4c(F)cccc4F)n3)ccc3nn(C45CC(C)(C4)C5)cc23)C[C@@H]1N]

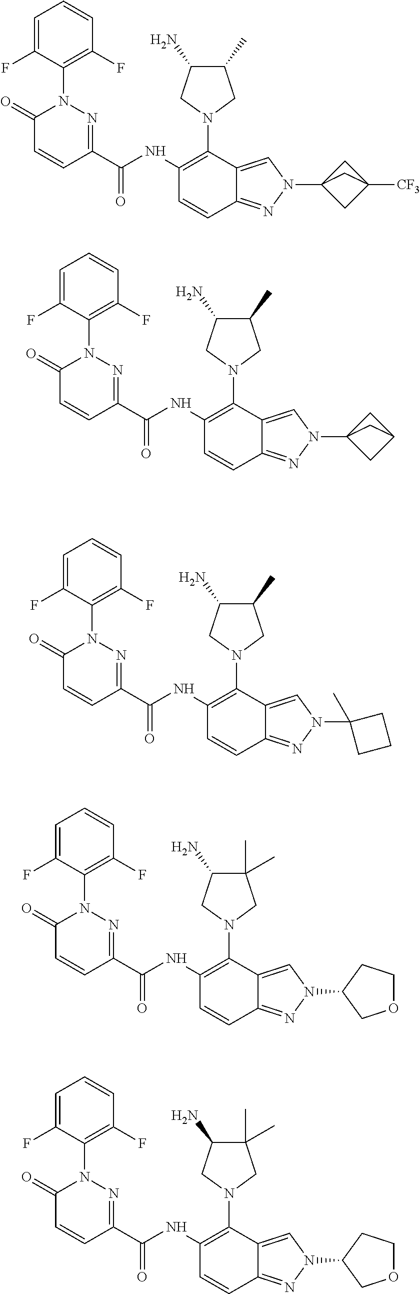 [SMILES: C[C@@H]1CN(c2c(NC(=O)c3ccc(=O)n(-c4c(F)cccc4F)n3)ccc3nn(C45CC(C(F)(F)F)(C4)C5)cc23)C[C@@H]1N ; C[C@H]1CN(c2c(NC(=O)c3ccc(=O)n(-c4c(F)cccc4F)n3)ccc3nn(C45CC(C4)C5)cc23)C[C@@H]1N ; C[C@H]1CN(c2c(NC(=O)c3ccc(=O)n(-c4c(F)cccc4F)n3)ccc3nn(C4(C)CCC4)cc23)C[C@@H]1N ; CC1(C)CN(c2c(NC(=O)c3ccc(=O)n(-c4c(F)cccc4F)n3)ccc3nn([C@@H]4CCOC4)cc23)C[C@@H]1N ; CC1(C)CN(c2c(NC(=O)c3ccc(=O)n(-c4c(F)cccc4F)n3)ccc3nn([C@@H]4CCOC4)cc23)C[C@H]1N]

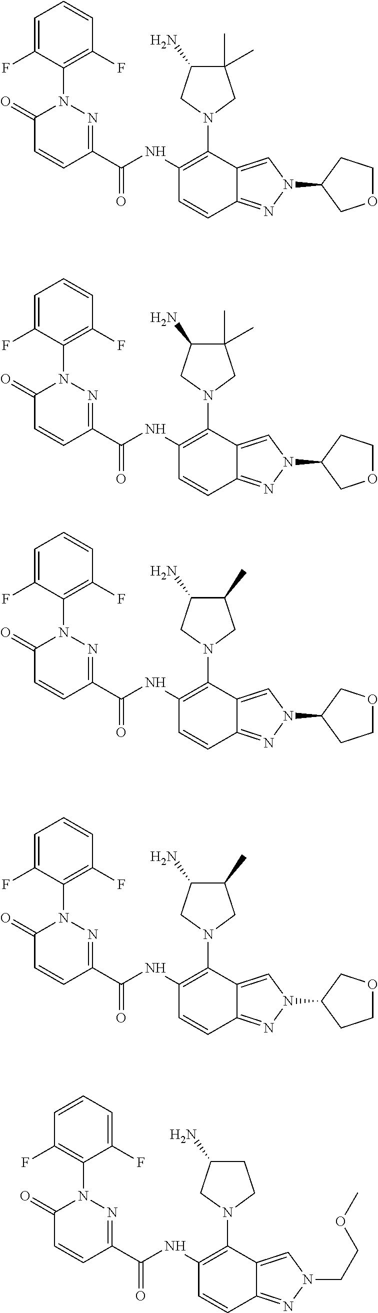 [SMILES: CC1(C)CN(c2c(NC(=O)c3ccc(=O)n(-c4c(F)cccc4F)n3)ccc3nn([C@H]4CCOC4)cc23)C[C@@H]1N ; CC1(C)CN(c2c(NC(=O)c3ccc(=O)n(-c4c(F)cccc4F)n3)ccc3nn([C@H]4CCOC4)cc23)C[C@H]1N ; C[C@H]1CN(c2c(NC(=O)c3ccc(=O)n(-c4c(F)cccc4F)n3)ccc3nn([C@@H]4CCOC4)cc23)C[C@@H]1N ; C[C@H]1CN(c2c(NC(=O)c3ccc(=O)n(-c4c(F)cccc4F)n3)ccc3nn([C@H]4CCOC4)cc23)C[C@@H]1N ; COCCn1cc2c(N3CC[C@@H](N)C3)c(NC(=O)c3ccc(=O)n(-c4c(F)cccc4F)n3)ccc2n1]

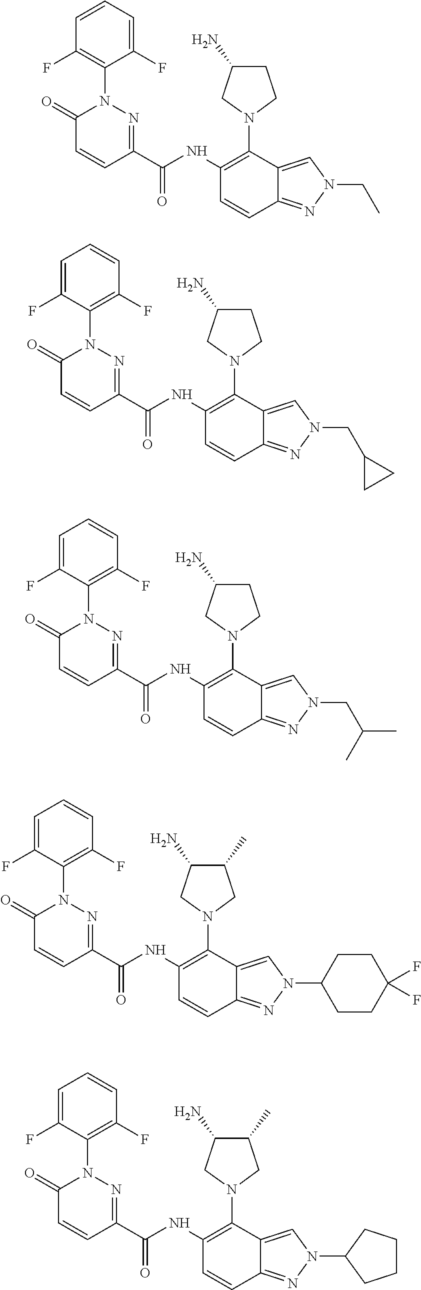 [SMILES: CCn1cc2c(N3CC[C@@H](N)C3)c(NC(=O)c3ccc(=O)n(-c4c(F)cccc4F)n3)ccc2n1 ; N[C@@H]1CCN(c2c(NC(=O)c3ccc(=O)n(-c4c(F)cccc4F)n3)ccc3nn(CC4CC4)cc23)C1 ; CC(C)Cn1cc2c(N3CC[C@@H](N)C3)c(NC(=O)c3ccc(=O)n(-c4c(F)cccc4F)n3)ccc2n1 ; C[C@@H]1CN(c2c(NC(=O)c3ccc(=O)n(-c4c(F)cccc4F)n3)ccc3nn(C4CCC(F)(F)CC4)cc23)C[C@@H]1N ; C[C@@H]1CN(c2c(NC(=O)c3ccc(=O)n(-c4c(F)cccc4F)n3)ccc3nn(C4CCCC4)cc23)C[C@@H]1N]

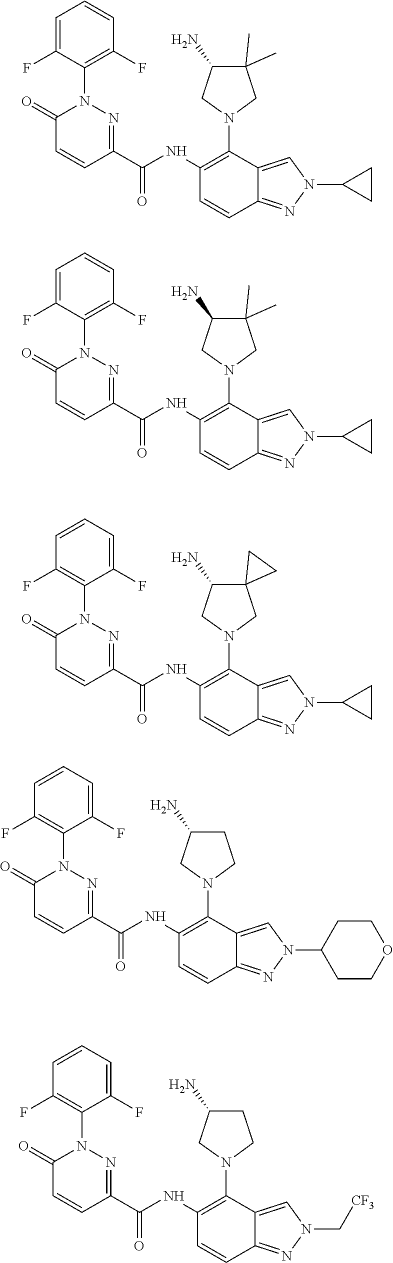 [SMILES: CC1(C)CN(c2c(NC(=O)c3ccc(=O)n(-c4c(F)cccc4F)n3)ccc3nn(C4CC4)cc23)C[C@@H]1N ; CC1(C)CN(c2c(NC(=O)c3ccc(=O)n(-c4c(F)cccc4F)n3)ccc3nn(C4CC4)cc23)C[C@H]1N ; N[C@H]1CN(c2c(NC(=O)c3ccc(=O)n(-c4c(F)cccc4F)n3)ccc3nn(C4CC4)cc23)CC12CC2 ; N[C@@H]1CCN(c2c(NC(=O)c3ccc(=O)n(-c4c(F)cccc4F)n3)ccc3nn(C4CCOCC4)cc23)C1 ; N[C@@H]1CCN(c2c(NC(=O)c3ccc(=O)n(-c4c(F)cccc4F)n3)ccc3nn(CC(F)(F)F)cc23)C1]

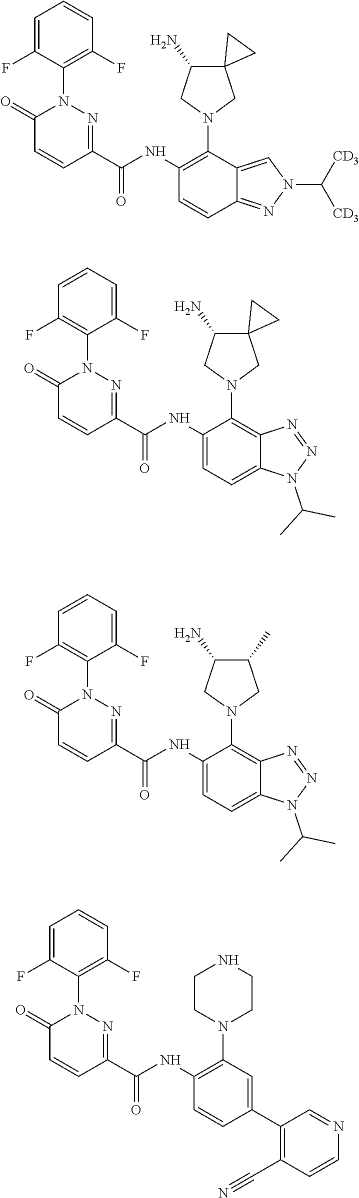 [SMILES: [2H]C([2H])([2H])C(n1cc2c(N3C[C@H](N)C4(CC4)C3)c(NC(=O)c3ccc(=O)n(-c4c(F)cccc4F)n3)ccc2n1)C([2H])([2H])[2H] ; CC(C)n1nnc2c(N3C[C@H](N)C4(CC4)C3)c(NC(=O)c3ccc(=O)n(-c4c(F)cccc4F)n3)ccc21 ; CC(C)n1nnc2c(N3C[C@@H](C)[C@@H](N)C3)c(NC(=O)c3ccc(=O)n(-c4c(F)cccc4F)n3)ccc21 ; N#Cc1ccncc1-c1ccc(NC(=O)c2ccc(=O)n(-c3c(F)cccc3F)n2)c(N2CCNCC2)c1]

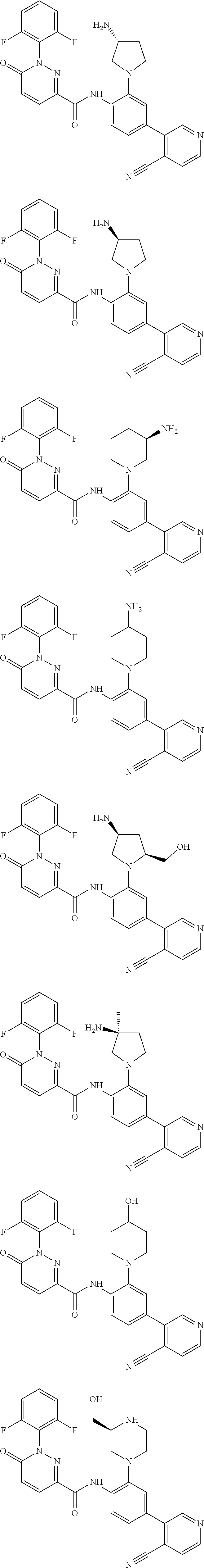

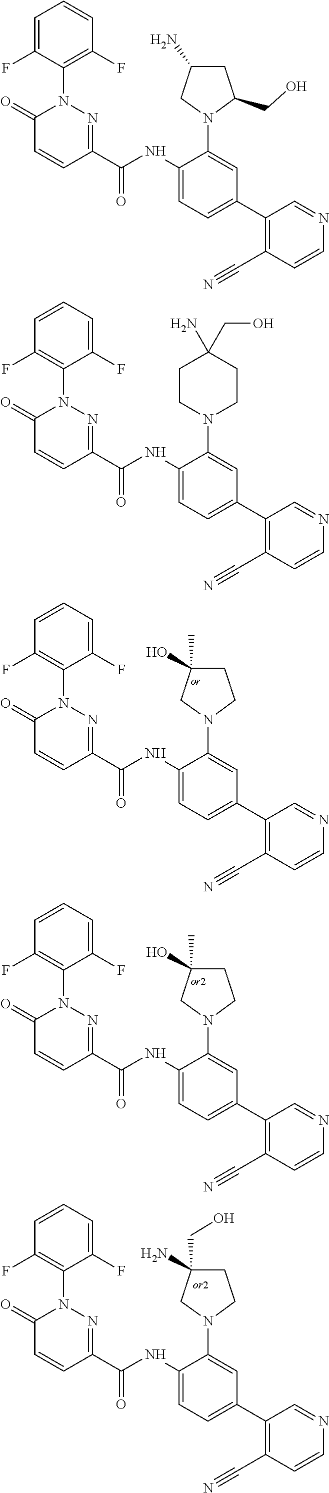

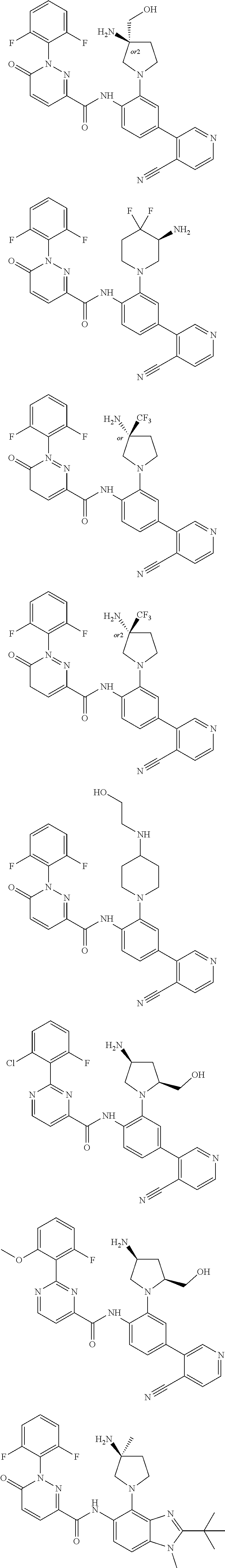

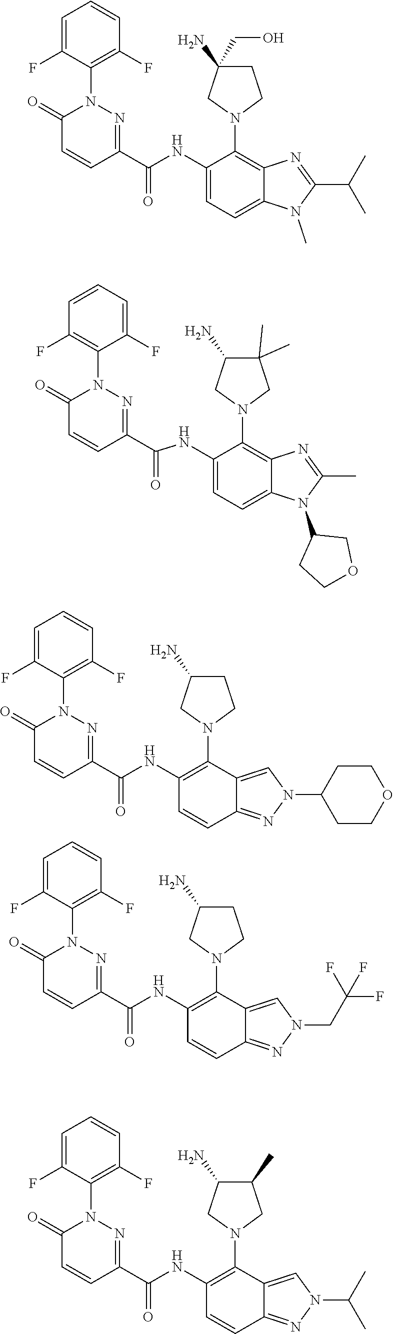

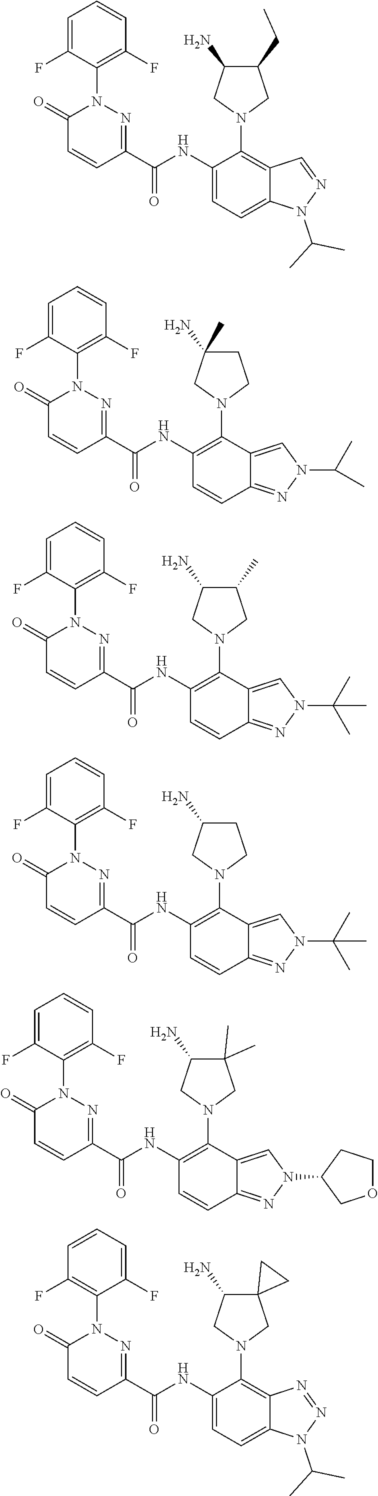

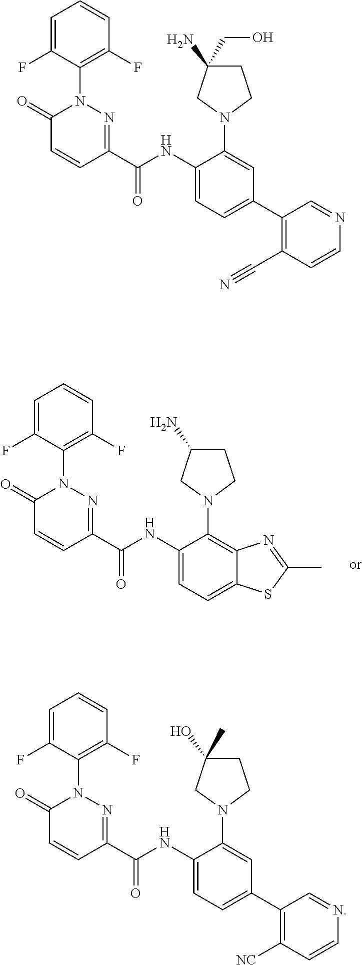 [SMILES: N#Cc1ccncc1-c1ccc(NC(=O)c2ccc(=O)n(-c3c(F)cccc3F)n2)c(N2CC[C@@](N)(CO)C2)c1 ; Cc1nc2c(N3CC[C@@H](N)C3)c(NC(=O)c3ccc(=O)n(-c4c(F)cccc4F)n3)ccc2s1 ; C[C@@]1(O)CCN(c2cc(-c3cnccc3C#N)ccc2NC(=O)c2ccc(=O)n(-c3c(F)cccc3F)n2)C1]

14. The compound of claim 1, wherein the compound is

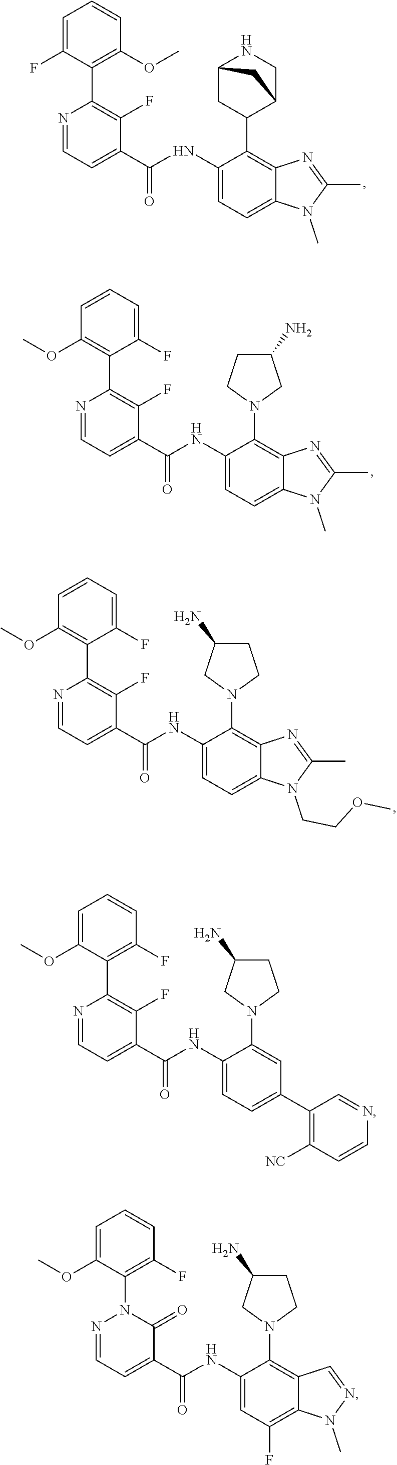

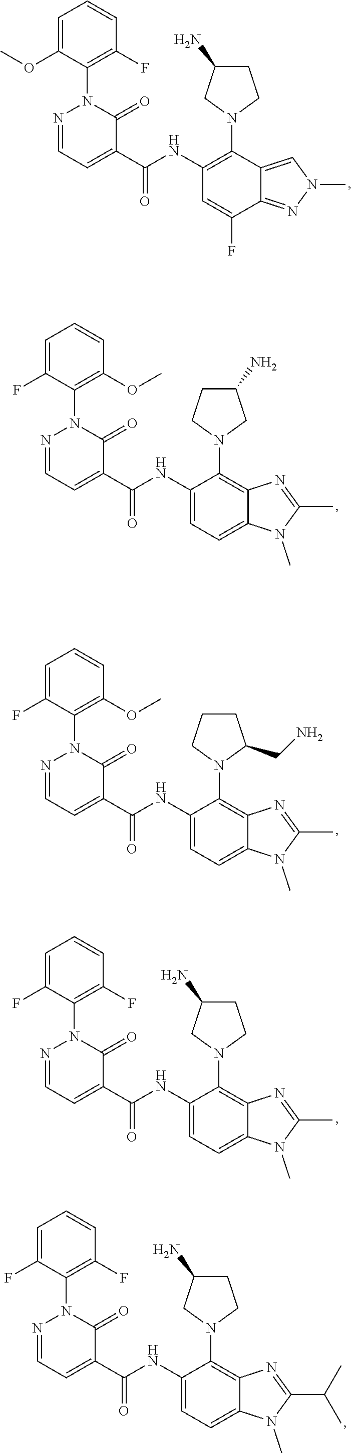

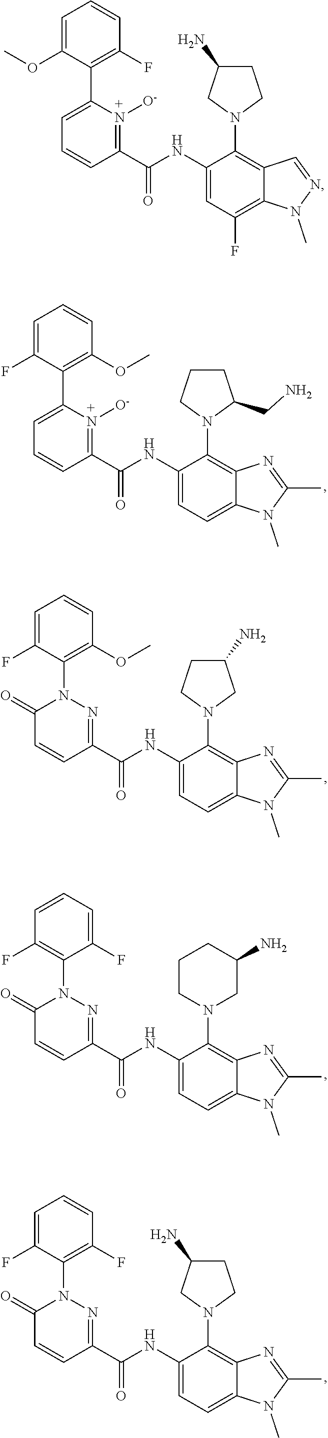

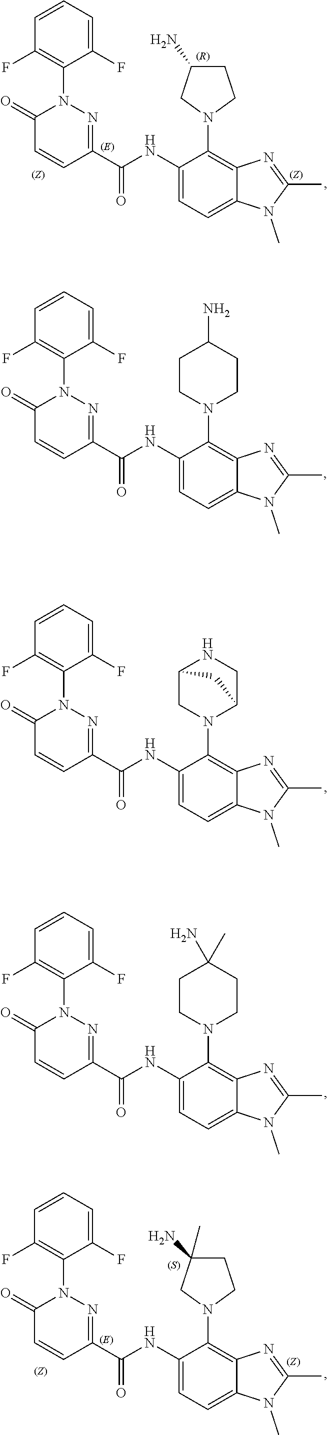

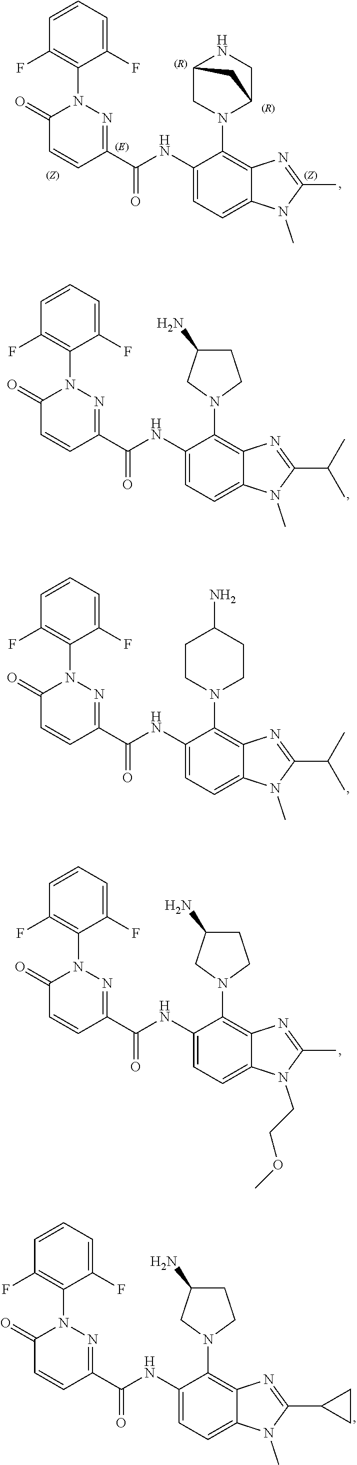

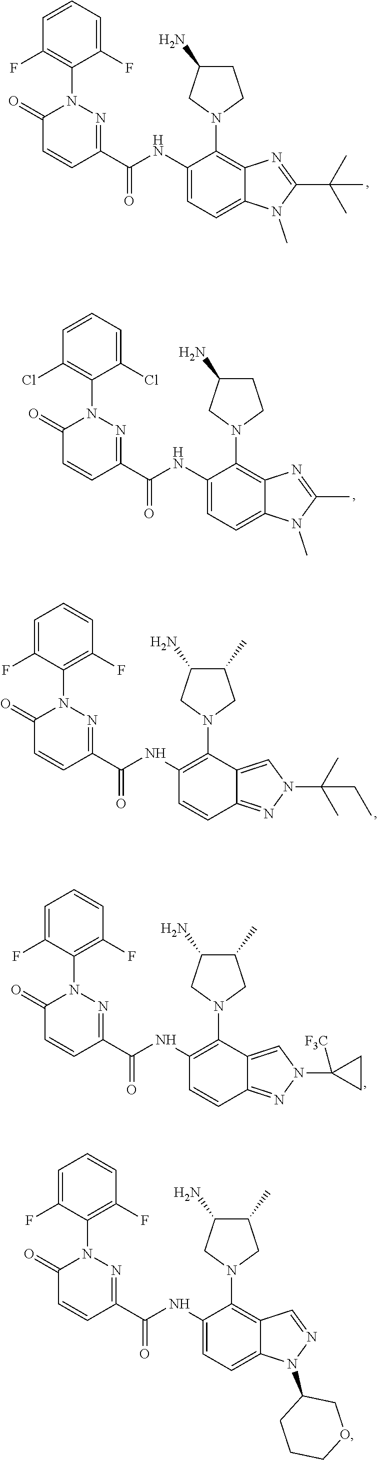 [SMILES: Cn1c(C(C)(C)C)nc2c(N3CC[C@H](N)C3)c(NC(=O)c3ccc(=O)n(-c4c(F)cccc4F)n3)ccc21 ; Cc1nc2c(N3CC[C@H](N)C3)c(NC(=O)c3ccc(=O)n(-c4c(Cl)cccc4Cl)n3)ccc2n1C ; CCC(C)(C)n1cc2c(N3C[C@@H](C)[C@@H](N)C3)c(NC(=O)c3ccc(=O)n(-c4c(F)cccc4F)n3)ccc2n1 ; C[C@@H]1CN(c2c(NC(=O)c3ccc(=O)n(-c4c(F)cccc4F)n3)ccc3nn(C4(C(F)(F)F)CC4)cc23)C[C@@H]1N ; C[C@@H]1CN(c2c(NC(=O)c3ccc(=O)n(-c4c(F)cccc4F)n3)ccc3c2cnn3[C@@H]2CCCOC2)C[C@@H]1N]

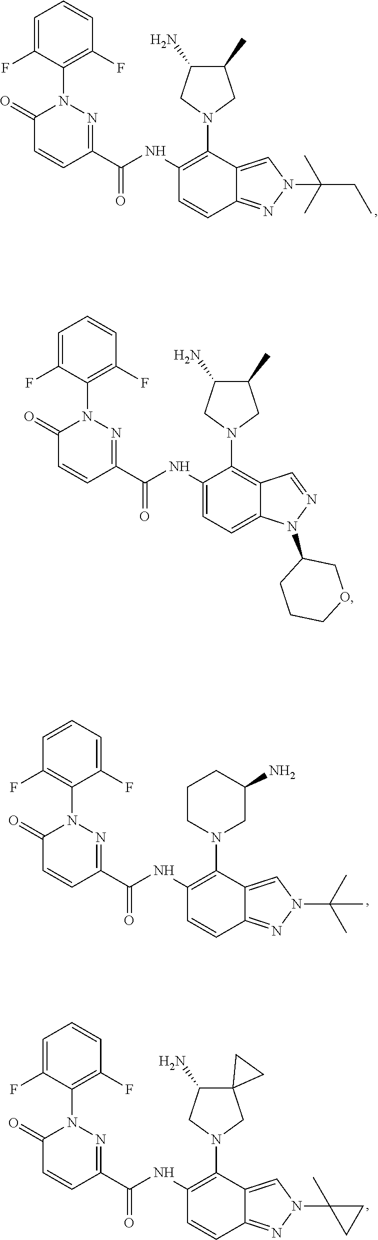 [SMILES: CCC(C)(C)n1cc2c(N3C[C@H](C)[C@@H](N)C3)c(NC(=O)c3ccc(=O)n(-c4c(F)cccc4F)n3)ccc2n1 ; C[C@H]1CN(c2c(NC(=O)c3ccc(=O)n(-c4c(F)cccc4F)n3)ccc3c2cnn3[C@@H]2CCCOC2)C[C@@H]1N ; CC(C)(C)n1cc2c(N3CCC[C@@H](N)C3)c(NC(=O)c3ccc(=O)n(-c4c(F)cccc4F)n3)ccc2n1 ; CC1(n2cc3c(N4C[C@H](N)C5(CC5)C4)c(NC(=O)c4ccc(=O)n(-c5c(F)cccc5F)n4)ccc3n2)CC1]

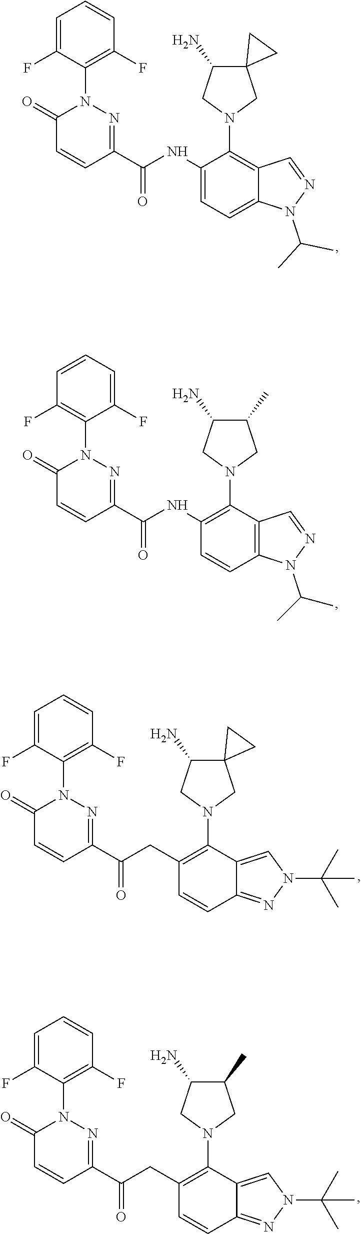 [SMILES: CC(C)n1ncc2c(N3C[C@H](N)C4(CC4)C3)c(NC(=O)c3ccc(=O)n(-c4c(F)cccc4F)n3)ccc21 ; CC(C)n1ncc2c(N3C[C@@H](C)[C@@H](N)C3)c(NC(=O)c3ccc(=O)n(-c4c(F)cccc4F)n3)ccc21 ; CC(C)(C)n1cc2c(N3C[C@H](N)C4(CC4)C3)c(CC(=O)c3ccc(=O)n(-c4c(F)cccc4F)n3)ccc2n1 ; C[C@H]1CN(c2c(CC(=O)c3ccc(=O)n(-c4c(F)cccc4F)n3)ccc3nn(C(C)(C)C)cc23)C[C@@H]1N]

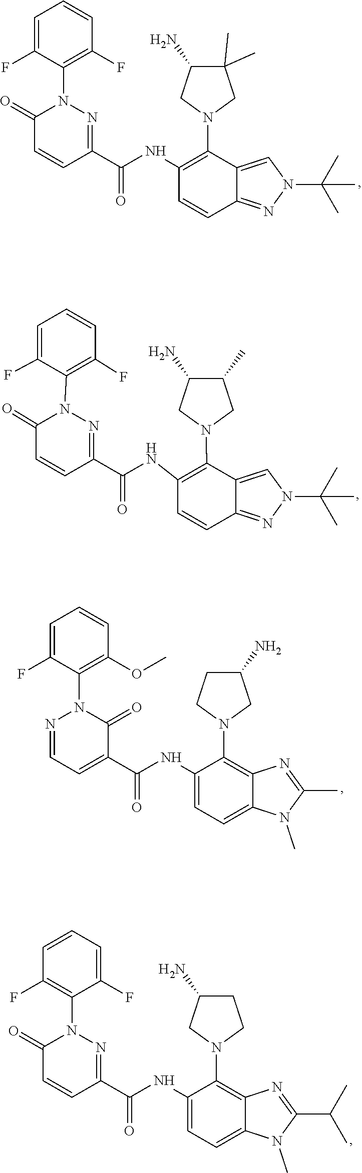 [SMILES: CC1(C)CN(c2c(NC(=O)c3ccc(=O)n(-c4c(F)cccc4F)n3)ccc3nn(C(C)(C)C)cc23)C[C@@H]1N ; C[C@@H]1CN(c2c(NC(=O)c3ccc(=O)n(-c4c(F)cccc4F)n3)ccc3nn(C(C)(C)C)cc23)C[C@@H]1N ; COc1cccc(F)c1-n1nccc(C(=O)Nc2ccc3c(nc(C)n3C)c2N2CC[C@H](N)C2)c1=O ; CC(C)c1nc2c(N3CC[C@@H](N)C3)c(NC(=O)c3ccc(=O)n(-c4c(F)cccc4F)n3)ccc2n1C]

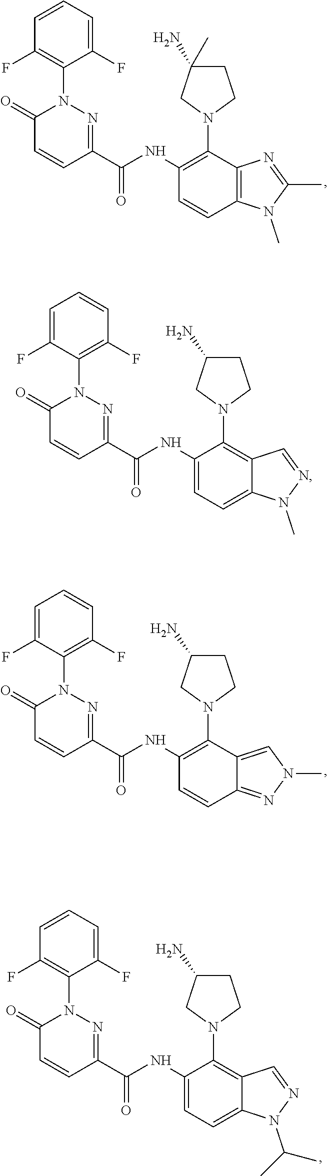 [SMILES: Cc1nc2c(N3CC[C@@](C)(N)C3)c(NC(=O)c3ccc(=O)n(-c4c(F)cccc4F)n3)ccc2n1C ; Cn1ncc2c(N3CC[C@@H](N)C3)c(NC(=O)c3ccc(=O)n(-c4c(F)cccc4F)n3)ccc21 ; Cn1cc2c(N3CC[C@@H](N)C3)c(NC(=O)c3ccc(=O)n(-c4c(F)cccc4F)n3)ccc2n1 ; CC(C)n1ncc2c(N3CC[C@@H](N)C3)c(NC(=O)c3ccc(=O)n(-c4c(F)cccc4F)n3)ccc21]

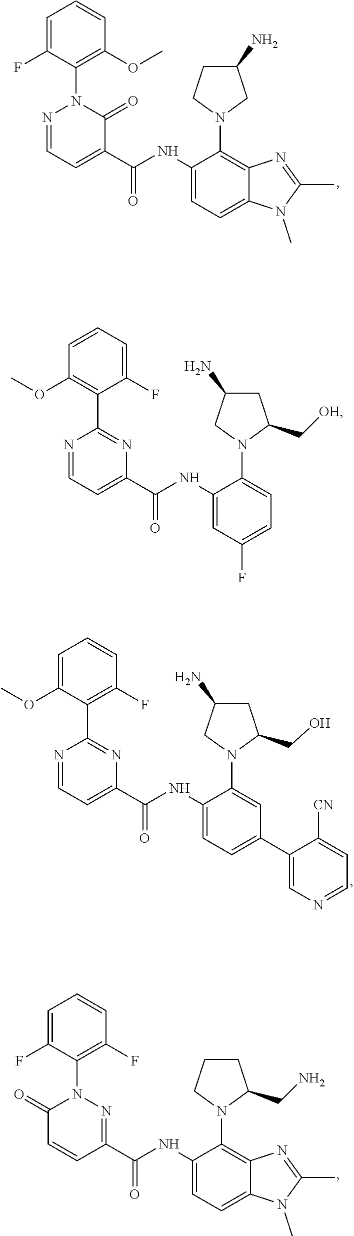

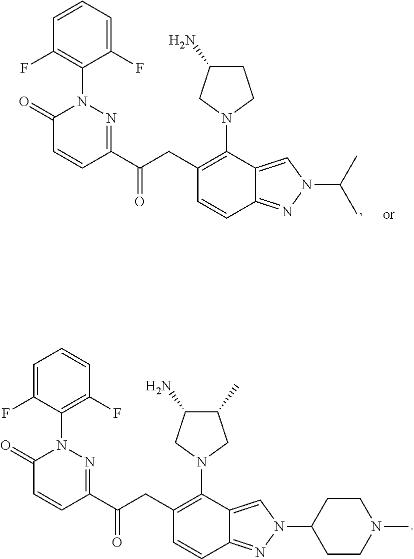 [SMILES: CC(C)n1cc2c(N3CC[C@@H](N)C3)c(CC(=O)c3ccc(=O)n(-c4c(F)cccc4F)n3)ccc2n1 ; C[C@@H]1CN(c2c(CC(=O)c3ccc(=O)n(-c4c(F)cccc4F)n3)ccc3nn(C4CCN(C)CC4)cc23)C[C@@H]1N]

15. A pharmaceutical composition comprising a compound of claim 1, and a pharmaceutically acceptable carrier or excipient.

16. The pharmaceutical composition of claim 15, further comprising a second therapeutic agent.

17. The pharmaceutical composition of claim 16, wherein the second therapeutic agent is an immune-checkpoint inhibitor or an inhibitor of the PGE2 or adenosine pathway.

18. The pharmaceutical composition of claim 17, wherein the immune-checkpoint inhibitor is ipilimumab, nivolumab, or pembroluzimab.

19. A method for treating a disorder mediated by HPK1 in a subject in need thereof, comprising administering to the subject a therapeutically effective amount of a compound of claim 1.

20. The method of claim 19, wherein the disorder is cancer.

21. The method of claim 20, wherein the cancer is breast cancer, colorectal cancer, lung cancer, ovarian cancer, or pancreatic cancer.

22. (canceled)
